SEQUENCE LISTING (17 sequences)

SEQ ID NO: 1 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 2 - protein, 323 aa, Macaca fascicularis
MARGPGLAPPPLRLPLLLLLLAAVTGHTAAQDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTASAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLTAGLIAVIVVVVVALVAGVAVLVISNRRKSGKYKKVEIKELGELRKEPSL

SEQ ID NO: 3 - protein, 317 aa, Mus musculus
MARGLDLAPLLLLLLAMATRFCTAQSNCTCPTNKMTVCDTNGPGGVCQCRAMGSQVLVDCSTLTSKCLLLKARMSARKSGRSLVMPSEHAILDNDGLYDPECDDKGRFKARQCNQTSVCWCVNSVGVRRTDKGDQSLRCDEVVRTHHILIELRHRPTDRAFNHSDLDSELRRLFQERYKLHPSFLSAVHYEEPTIQIELRQNASQKGLRDVDIADAAYYFERDIKGESLFMGRRGLDVQVRGEPLHVERTLIYYLDEKPPQFSMKRLTAGVIAVIAVVSVAVVAGVVVLVVTKRRKSGKYKKVELKELGEMRSEPSL

SEQ ID NO: 4 - protein, 5 aa, synthetic construct
SNGIS

SEQ ID NO: 5 - protein, 16 aa, synthetic construct
VIDSYGTTYYADSVKG

SEQ ID NO: 6 - protein, 14 aa, synthetic construct
DLSYSSVMGYGMDP

SEQ ID NO: 7 - protein, 11 aa, synthetic construct
RASQSINNYLA

SEQ ID NO: 8 - protein, 7 aa, synthetic construct
SASYLAS

SEQ ID NO: 9 - protein, 10 aa, synthetic construct
QSTAGISDND

SEQ ID NO: 10 - protein, 123 aa, synthetic construct
MQVQLVESGGGVVQPGRSLRLSCAVSGFSLSSNGISWVRQAPGKGLEWVAVIDSYGTTYYADSVKGRFTISRDNSKHTVYLQMNSLRAEDTAVYYCARDLSYSSVMGYGMDPWGQGTLVTVSS

SEQ ID NO: 11 - protein, 109 aa, synthetic construct
MDIQMTQSPSSLSASVGDRVTITCRASQSINNYLAWYQQKPGXPKLLIYSASYLASGVPSRFSGSGSGTDFTLTISSLQPEDFATYYCQSTAGISDNDFGGGTKVEIKR

SEQ ID NO: 12 - protein, 453 aa, synthetic construct
MQVQLVESGGGVVQPGRSLRLSCAVSGFSLSSNGISWVRQAPGKGLEWVAVIDSYGTTYYADSVKGRFTISRDNSKHTVYLQMNSLRAEDTAVYYCARDLSYSSVMGYGMDPWGQGTLVTVSSASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKKVEPKSCDKTHTCPPCPAPELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 13 - protein, 453 aa, synthetic construct
MQVQLVESGGGVVQPGRSLRLSCAVSGFSLSSNGISWVRQAPGKGLEWVAVIDSYGTTYYADSVKGRFTISRDNSKHTVYLQMNSLRAEDTAVYYCARDLSYSSVMGYGMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVXLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 14 - protein, 216 aa, synthetic construct
MDIQMTQSPSSLSASVGDRVTITCRASQSINNYLAWYQQKPGXAPKLLIYSASYLASGVPSRFSGSGSGTDFTLTISSLQPEDFATYYCQSTAGISDNDFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 452 aa, synthetic construct
MEVQLVESGGGLVQPGGSLRLSCAASGFNISNKWIHWVRQAPGKGLEWVGAIAPSSGDTDYADSVKGRFTISADTSKNTAYLQMNSLRAEDTAVYYCAREGYNAYWYVLDYWGQGTLVTVSSASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKKVEPKSCDKTHTCPPCPAPELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 16 - protein, 452 aa, synthetic construct
MEVQLVESGGGLVQPGGSLRLSCAASGFNISNKWIHWVRQAPGKGLEWVGWIAPSSGDTDYADSVKGRFTISADTSKNTAYLQMNSLRAEDTAVYYCAREGYNAYWYVLDYWGQGTLVTVSSASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKKVEPKSCDKTHTCPPCPAPELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSXFLYSKLTVDKSRWQQGNVFSCSVMHEALHNHYTQKSLSLSPGK

SEQ ID NO: 17 - protein, 215 aa, synthetic construct
MDIQMTQSPSSLSASVGDRVTITCRASQDVNTAVAWYQQKPGXAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0053942 A1
(43) Pub. Date: Feb. 26, 2026

(54) STING AGONIST IMMUNOSTIMULATORY ANTIBODY DRUG CONJUGATES

(71) Applicant: ASTELLAS PHARAMA INC., Tokyo (JP)

(72) Inventors: Gang Yin, South San Francisco, CA (US); Xiaofan Li, Belmont, CA (US); Helena Kiefel, South San Francisco, CA (US); Miao Wen, Union City, CA (US); Kshama Doshi, South San Francisco, CA (US); Wadie Daniel Mahauad Fernandez, Palo Alto, CA (US); Werner Rubas, Redwood City, CA (US); Junhao Yang, Palo Alto, CA (US); Amandeep Kaur Gakhal, San Mateo, CA (US); Ravi Prakash Singh, Burlingame, CA (US); Huiyong Hu, Fremont, CA (US); Wen Liu, Foster City, CA (US); Krishna Bajjuri, Union City, CA (US); Xiangwei Zhu, Albany, CA (US)

(21) Appl. No.: 19/222,730

(22) Filed: May 29, 2025

Related U.S. Application Data
(60) Provisional application No. 63/653,575, filed on May 30, 2024.

(51) Int. Cl. A61K 47/68; A61P 35/00; C07K 16/30

(52) U.S. Cl. CPC A61K 47/6851; A61K 47/6803; A61K 47/6889; A61P 35/00; C07K 16/30

(57) ABSTRACT

The present disclosure is related to dual payload immunostimulatory antibody drug conjugates comprising at least STING agonist and at least one cytotoxic agent, pharmaceutical compositions thereof, and the use of the dual payload immunostimulatory antibody drug conjugates and compositions thereof for the treatment of diseases and disorders, including proliferative diseases

### CROSS-REFERENCE TO RELATED APPLICATIONS

This application claims priority to Provisional Patent Application No. 63/653,575 filed May 30, 2024, the entirety of which is incorporated by reference for all purposes.

### REFERENCE TO SEQUENCE LISTING SUBMITTED VIA EFS-WEB

This application includes an electronically submitted sequence listing in .XML format. The .XML file contains a sequence listing entitled “128696_00002 Sequence Listing” created on May 29, 2025, and is 18,873 bytes in size. The sequence listing contained in this .XML file is part of the specification and is incorporated herein by reference in its entirety.

### FIELD

The present disclosure is related to dual payload immunostimulatory antibody drug conjugates comprising at least one STING agonist and at least one cytotoxic agent, pharmaceutical compositions thereof, and the use of the dual payload immunostimulatory antibody drug conjugates and compositions thereof for the treatment of diseases and disorders, including proliferative diseases.

### BACKGROUND

Cancer treatments commonly employ combinations of drugs that may display therapeutic activity by acting through complementary mechanisms of action. For instance, cytotoxic drugs are often employed in combination with adjuvants and/or immune system modulators. However, conventional cytotoxic drugs are not tumor specific and are often associated with side effects.

One way to circumvent the non-specificity of cytotoxic drugs is to conjugate the cytotoxic drug to an antibody because tumor cells express specific antigens that can be targeted by the antibody. Additionally, if an immune response can be induced at the same time, the immune system can recruit immune cells to target tumors expressing the specific antigens to boost the therapeutic efficacy of the antibody-drug conjugate cancer treatment. Further, the immune response may not be limited to targeting only tumor cells that express those tumor antigens; tumor cells in the periphery may also be targeted via antigen spreading and/or through less specific mechanisms of tumor cell killing (e.g., cytokine release).

One protein that is important to induce immunity is stimulator of interferon genes (STING). In particular, the cGAS-STING pathway helps to detect and protect against harmful cytosolic DNA, which is an indication of cellular and tissue damage (because the cytosol is normally free of DNA, leaked cytosolic DNA is often indicative of DNA damage events and tumorigenesis). cGAS recognizes cytosolic DNA and catalyzes the synthesis of cyclic dinucleotides (CDNs), including cGAMP, which in turn bind and activate STING. Once STING is bound to a CDN, STING undergoes a conformational change, translocates from the endoplasmic reticulum to the Golgi apparatus, and triggers the transcription factor TBK1 to phosphorylate transcription factors interferon regulatory factor 3 (IRF3) and nuclear factor κB (NF-κB). This induces type I interferons (IFNs), immune-stimulated genes, and the production of pro-inflammatory cytokines, such as IL-6, TNF-α, and IFN-γ.

It is well-established that IFNs can inhibit tumor cell proliferation via multiple mechanisms. As described in Jiang, M. et al. Journal of Hematology & Oncology, 2020, 81, 13, STING-deficiency is correlated with cancer incidence in at least melanoma cell lines, colorectal adenocarcinoma human cell lines, and lung cancer.

A number of STING agonists have been developed and studied for oncological indications (Le Naour et al. Oncoimmunology, 2020; 9(1): 1777624), including DMXAA (or Vadimezan), a tumor-vascular disrupting agent that has been studied in clinical trials for its effect on advanced solid tumors, prostate cancer, urothelial carcinoma, and small cell lung cancer. Despite promising preclinical results, DMXAA has thus far only yielded poor results in human clinical trials. MIW815 (ADU-S100) in combination with pembrolizumab was recently studied in a Phase 2 clinical trial for patients with head and neck cancer, but was terminated due to a lack of substantial anti-tumor activity (NCT03937141). A Phase 1 trial to study the effect of MIW815 as a single agent and in combination with ipilimumab in patients with advanced/metastatic solid tumors or lymphomas (NCT02675439) was also terminated for showing a lack of substantial anti-tumor activity.

Other STING agonists include diamidobenzimidazole (di-ABZI) STING agonists, for example, those described in U.S. Pat. No. 11,377,440. U.S. Pat. No. 11,155,567 describes di-ABZI STING agonists, including XMT-2056, however, a Phase I clinical trial of XMT-2056 for HER2+ recurrent or metastatic solid tumors was on temporary clinical hold in March 2023 following a patient death (NCT05514717). Additional di-ABZI STING agonists are described in PCT Applications WO 2020/042995; WO 2020/156363; WO 2023/025256; WO 2021/013250; and WO 2022/272039.

Given the importance of the STING pathway in inducing an immune response in response to damaged DNA associated with cellular proliferative disorders, there is a medical need to develop STING agonists that can be used safely and effectively in combination with cytotoxic and chemotherapeutic agents.

### SUMMARY

Described herein are dual payload immunostimulatory antibody drug conjugates (iADCs) comprising an anti-TROP2 antibody or antigen binding fragment thereof wherein the anti-TROP2 antibody or antigen binding fragment thereof is linked to at least one STING agonist via a first linker, and the anti-TROP2 antibody or antigen binding fragment thereof is also linked to at least one cytotoxic agent via a second linker. In certain embodiments, the at least one STING agonist and/or the at least one cytotoxic agent are covalently linked to the anti-TROP2 antibody or antigen-binding fragment thereof. In certain embodiments, the immunostimulatory antibody drug conjugate comprises an antibody that specifically binds to TROP2, and the antibody is linked site-specifically to at least one STING agonist, and the antibody is linked site-specifically to at least one cytotoxic agent.

In some embodiments, the iADC is represented by the structure of Formula (I):

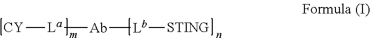

or a pharmaceutically acceptable salt, solvate, stereoisomer, regioisomer, or mixture of regioisomers thereof,
wherein Ab is an anti-TROP2 antibody or antigen-binding fragment thereof,
L^{a} is a first linker;
L^{b} is a second linker;
CY is independently a residue of a cytotoxic agent;
STING is independently a residue of a STING agonist;
n is an integer selected from 1 to 10; and
m is an integer selected from 1 to 10.

In another aspect, provided herein are pharmaceutical compositions comprising the iADCs described herein. In a further aspect, provided herein are kits comprising the iADCs described herein or pharmaceutical compositions thereof.

In another aspect, provided herein is a method for treating diseases, disorders, or conditions, including cancer and proliferative diseases, comprising administering an iADC described herein or a pharmaceutical composition thereof to a subject in need thereof. In some embodiments, the disease, disorder, or condition is cancer.

### BRIEF DESCRIPTION OF FIGURES

FIG. 1 provides a comparison of the Kabat and Chothia numbering systems for CDR-H1. Adapted from Martin A. C. R. (2010). Protein Sequence and Structure Analysis of Antibody Variable Domains. In R. Kontermann & S. Dubel (Eds.), Antibody Engineering vol. 2 (pp. 33-51). Springer-Verlag, Berlin Heidelberg.

FIG. 2 is a graph of the anti-tumor activity of ADC 1, ADC 2, and ADC 4 in a syngeneic mouse tumor model until day 18 post treatment.

FIG. 3A is a graph of the anti-tumor activity of iADC 1, iADC 2, and iADC 3, administered at a dose of 2 mg/kg, in a syngeneic mouse tumor model compared to ADC 4, also administered at a dose of 2 mg/kg.

FIG. 3B is a graph of the anti-tumor activity of iADC 1, iADC 2, and iADC 3, administered at a dose of 1 mg/kg, in a syngeneic mouse tumor model.

FIG. 4A is a graph of the anti-tumor activity of iADC 15 and iADC 18, administered at a dose of 2 mg/kg, in a syngeneic mouse tumor model compared to ADC 4, also administered at a dose of 2 mg/kg.

FIG. 4B is a graph of the anti-tumor activity of iADC 1, iADC 9, iADC 15, and iADC 18, administered at a dose of 1 mg/kg, in a syngeneic mouse tumor model.

FIG. 4C is a graph of the anti-tumor activity of iADC 16 and iADC 17, administered at a dose of 2 mg/kg, in a syngeneic mouse tumor model compared to ADC 4, also administered at a dose of 2 mg/kg.

FIG. 4D is a graph of the anti-tumor activity of iADC 1, iADC 7, iADC 16, and iADC 17, administered at a dose of 1 mg/kg, in a syngeneic mouse tumor model.

FIG. 5A is a graph of the anti-tumor activity of iADC 1, iADC 9 and iADC 17, administered at a dose of 1 mg/kg, in a syngeneic mouse tumor model compared to ADC 4 and ADC 6, also administered at a dose of 1 mg/kg.

FIG. 5B is a graph of the anti-tumor activity of iSAC 1 and iADC 1, administered at a dose of 0.5 mg/kg, in a syngeneic mouse tumor model.

FIG. 5C is a graph of the anti-tumor activity of iSAC 1 and iADC 1, administered at a dose of 1 mg/kg, in a syngeneic mouse tumor model compared to ADC 4, also administered at a dose of 1 mg/kg.

FIG. 5D is a graph of the anti-tumor activity of iADC 17 and iSAC 14, administered at a dose of 1 mg/kg, in a syngeneic mouse tumor model compared to ADC 4, also administered at a dose of 1 mg/kg.

FIG. 5E is a graph of the anti-tumor activity of iADC 9 and iSAC 2, administered at a dose of 1 mg/kg, in a syngeneic mouse tumor model compared to ADC 6, also administered at a dose of 1 mg/kg.

FIG. 6A is a graph measuring the IFNβ in plasma in a syngeneic mouse tumor model following administration of iADC 1, iADC 9, and iADC 17, administered at a dose of 1 mg/kg.

FIG. 6B is a graph measuring the TNFα in plasma in a syngeneic mouse tumor model following administration of iADC 1, iADC 9, and iADC 17, administered at a dose of 1 mg/kg.

FIG. 6C is a graph measuring the GranzymeB positive NK cells in a syngeneic mouse tumor model following administration of iADC 1, iADC 9, and iADC 17, administered at a dose of 1 mg/kg.

FIG. 6D is a graph measuring the GranzymeB positive CD8-T cells in a syngeneic mouse tumor model following administration of iADC 1, iADC 9, and iADC 17, administered at a dose of 1 mg/kg.

FIG. 6E is a graph measuring the increase of monocytes in a syngeneic mouse tumor model following administration of iADC 1, iADC 9, and iADC 17, administered at a dose of 1 mg/kg.

### DETAILED DESCRIPTION

Described herein are dual payload immunostimulatory antibody drug conjugates (iADCs) comprising an anti-TROP2 antibody or antigen binding fragment thereof conjugated to at least one STING agonist and at least one cytotoxic agent.

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Green & Sambrook, Molecular Cloning: A Laboratory Manual 4^{th} ed. (2012), Cold Spring Harbor Laboratory Press, Cold Spring Harbor, NY; and Ausubel et al., Current Protocols in Molecular Biology, John Wiley & Sons. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value. In certain embodiments, for example, logarithmic scales (e.g., pH), the term “about” indicates the designated value ±0.3, ±0.2, or ±0.1.

The term “combinations thereof” includes every possible combination of elements to which the term refers to.

The term “TROP2” refers to, unless specified otherwise, any variants, isoforms and species homologs of human Trophoblast cell surface antigen 2 (TROP2) that are naturally expressed by cells, or that are expressed by cells transfected with a TROP2 or TROP2 gene. TROP2 proteins include, for example, human TROP2 (SEQ ID NO: 1). In some embodiments, TROP2 proteins include cynomolgus monkey TROP2 (SEQ ID NO: 2). In some embodiments, TROP2 proteins include murine TROP2 (SEQ ID NO: 3).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H} or VH) and a heavy chain constant region (C_{H} or CH). The heavy chain constant region typically comprises three domains, abbreviated C_{H}1 (or CH1), C_{H}2 (or CH2), and C_{H}3 (or CH3). Each light chain typically comprises a light chain variable region (V_{L} or VL) and a light chain constant region. The light chain constant region typically comprises one domain, abbreviated C_{L} or CL.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer.

The term “anti-TROP2 antibody” is an antibody, as described herein, which binds specifically to TROP2. In some embodiments, the antibody binds the extracellular domain of TROP2. As used herein, unless otherwise specified, an antibody may also comprise an “antibody fragment” as defined herewithin.

The V_{H} and V_{L} regions may be further subdivided into regions of hypervariability (“hypervariable regions (HVRs)”; also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgAQ1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme), each of which is incorporated by reference in its entirety.

Table A provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE A
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 | H31-H35 or | H26-H32 or |
| | H35B | H34* |
| H2 | H50-H65 | H52-H56 |
| H3 | H95-H102 | H95-H102 |

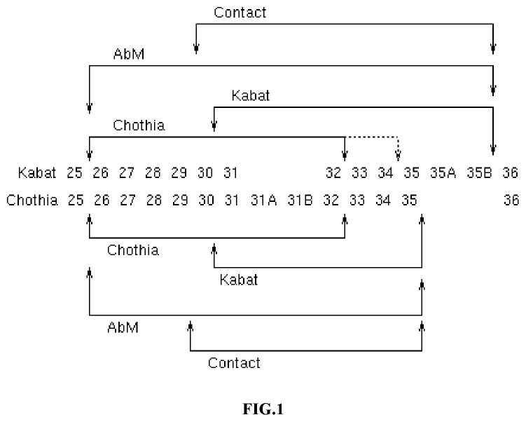

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat numbering scheme. Where the residues encompassed by these two numbering schemes diverge (e.g., CDR-H1 and/or CDR-H2), the numbering scheme is specified as Kabat. For convenience, CDR-H3 is sometimes referred to herein as Kabat. However, this is not intended to imply differences in sequence where they do not exist, and one of skill in the art can readily confirm whether the sequences are the same or different by examining the sequences.

CDRs may be assigned, for example, using antibody numbering software, such as Abnum, available at www.bioinforg.uk/abs/abnum/, and described in Abhinandan and Martin, Immunology, 2008, 45:3832-3839, incorporated by reference in its entirety.

The “framework region” refers to a subdivision of the variable region acting as a scaffold for the CDRs. Unless specified otherwise, there are four framework regions for each heavy chain and light chain, wherein each CDRs is flanked by one framework region. Unless stated otherwise, the numbering of the framework region is based on using the Kabat numbering scheme.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

An “affinity matured” antibody is one with one or more alterations in one or more CDRs or FRs that result in an improvement in the affinity of the antibody for its antigen, compared to a parent antibody which does not possess the alteration(s). In some embodiments, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

The term “amino acid” refers to the twenty common naturally occurring amino acids. Naturally occurring amino acids include pyrrolysine and selenocysteine. Natural amino acids also include citrulline. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids, and acylated amino acids. The term “amino acid” also includes “non-natural amino acids” and “modified amino acids.” The terms “non-natural amino acids” and “modified amino acids” are used herein interchangeably.

The term “non-natural amino acid” (or “unnatural amino acid”) or “synthetic amino acids” are α, β, γ, or δ amino acids, and includes but is not limited to, amino acids found in proteins, i.e., glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In certain embodiments, the amino acid is in the L-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. Unnatural amino acids are not proteinogenic amino acids, or post translationally modified variants thereof that either occur naturally or are chemically synthesized. In particular, the term unnatural amino acid refers to an amino acid that is not one of the 20 common amino acids or pyrrolysine or selenocysteine, or post translationally modified variants thereof. Non-limiting examples of unnatural amino acids include sulfoalanine, hydroxyproline (Hyp), beta-alanine, citrulline (Cit), ornithine (Orn), norleucine (Nle), 3-nitrotyrosine, nitroarginine, pyroglutamic acid (Pyr), naphtylalanine (Nal), 2,4-diaminobutyric acid (DAB), methionine sulfoxide, and methionine sulfone.

The term “post translationally modified” refers to an antibody expressed in a cell that is modified after translation. Examples of the post-translational modification include modification such as pyroglutamylation, glycosylation, oxidation, deamidation or glycation of glutamine or glutamic acid at the heavy chain N-terminal, and lysine deletion by cutting lysine at the heavy chain C-terminal with carboxypeptidase. It is known that such post-translational modification is caused in various antibodies (J. Pharm. Sci., 2008, Vol. 97, p. 2426-2447).

The term “modified amino acid” is an amino acid or non-natural amino acid that comprises a reactive group capable of forming a covalent bond to a linker payload. The reactive group can be an amino, carboxy, acetyl, hydrazino, hydrazido, hydroxylamine, semicarbazido, sulfanyl, azido or alkynyl group. Non-limiting examples of non-natural amino acids include p-acetyl-L-phenylalanine, O-methyl-L-tyrosine, 3-methyl-phenylalanine, O-4-allyl-L-tyrosine, 4-propyl-L-tyrosine, fluorinated phenylalanine, isopropyl-L-phenylalanine, p-azido-L-phenylalanine, p-acyl-L-phenylalanine, p-benzoyl-L-phenylalanine, p-iodo-phenylalanine, p-bromophenylalanine, p-amino-L-phenylalanine, isopropyl-L-phenylalanine, p-propargyloxy-phenylalanine, and p-azidomethyl-L-phenyl alanine.

The term “conjugate” or “antibody conjugate” refers to an antibody or antigen binding fragment thereof linked to one or more payload moieties. The antibody can be any antibody described herein. The payload can be any payload such as cytotoxic agents and immunostimulants described herein. The antibody can be directly linked to the payload via a covalent bond, or the antibody can be linked to the payload indirectly via a linker. Typically, the linker is covalently bonded to the antibody and also covalently bonded to the payload. The term “antibody drug conjugate” or “ADC” as used herein refers to an antibody or antigen binding fragment thereof directly or indirectly, via a linker, covalently bound to at least one cytotoxic agent. The term “immunostimulatory antibody conjugate” or “iSAC” as used herein refers to an antibody or antigen binding fragment thereof directly or indirectly, via a linker, covalently bound to at least one STING agonist. The term “immunomodulatory antibody drug conjugate” or “iADC” as used herein refers to a dual antibody or antigen binding fragment thereof directly or indirectly, via a linker, covalently bound to at least one STING agonist and at least one cytotoxic agent.

The term “epitope” means a portion of an antigen capable of specific binding to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the groups of amino acids provided in Tables B-D are, in some embodiments, considered conservative substitutions for one another.

TABLE B
Selected groups of amino acids that are considered conservative
substitutions for one another, in certain embodiments.

| | |
|---|---|
| Acidic Residues | D and E |
| Basic Residues | K, R, and H |
| Hydrophilic Uncharged | S, T, N, and Q |
| Residues | |
| Aliphatic Uncharged Residues | G, A, V, L, and I |
| Non-polar Uncharged Residues | C, M, and P |
| Aromatic Residues | F, Y, and W |
| Alcohol Group-Containing | S and T |
| Residues | |
| Aliphatic Residues | I, L, V, and M |
| Cycloalkenyl-associated | F, H, W, and Y |
| Residues | |
| Hydrophobic Residues | A, C, F, G, H, I, L, M, R, T, |
| | V, W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in | A, C, D, E, G, H, K, N, Q, R, |
| Turn Formation | S, P, and T |
| Flexible Residues | Q, T, K, S, G, P, D, E, and R |

TABLE C
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE D
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

“pAzF” mutation refers to a variant phenylalanine residue, i.e., para-azidomethyl-L-phenylalanine, added or substituted into a polypeptide.

“pAcF” mutation refers to a variant phenylalanine residue, i.e., para-acetyl-L-phenylalanine, added or substituted into a polypeptide.

“pAzF” mutation refers to a variant phenylalanine residue, i.e., para-azido-L-phenylalanine, added or substituted into a polypeptide.

The term “payload” refers to a molecular moiety that can be conjugated to an antibody. In particular embodiments, payloads are selected from the group consisting of therapeutic moieties, for example, the STING agonists and cytotoxic agents described herein.

The term “linker” refers to a molecular moiety that is capable of forming at least two covalent bonds. Typically, a linker is capable of forming at least one covalent bond to an antibody and at least another covalent bond to a payload. In certain embodiments, a linker can form more than one covalent bond to an antibody. In certain embodiments, a linker can form more than one covalent bond to a payload or can form covalent bonds to more than one payload. After a linker forms a bond to an antibody, or a payload, or both, the remaining structure, i.e., the residue of the linker after one or more covalent bonds are formed, may still be referred to as a “linker” herein. The term “linker precursor” refers to a linker having one or more reactive groups capable of forming a covalent bond with an antibody or payload, or both. In some embodiments, the linker is a cleavable linker. For example, a cleavable linker can be one that is released by a bio-labile function, which may or may not be engineered. In some embodiments, the linker is a non-cleavable linker. For example, a non-cleavable linker can be one that is released upon degradation of the antibody.

The term “EC₅₀” refers to a dosage, concentration, or amount of a particular test compound that elicits a dose-dependent response at 50% of maximal expression of a particular response that is induced, provoked, or potentiated by the particular test compound.

The term “IC₅₀” refers to an amount, concentration, or dosage of a particular test compound that achieves a 50% inhibition of a maximal response in an assay that measures such response.

The terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In another embodiment, the subject is a farm animal (e.g., a horse, a cow, a pig, etc.) or a pet (e.g., a dog or a cat). In certain embodiments, the subject is a human.

The terms “therapeutic agent” and “therapeutic agents” refer to any agent(s) which can be used in the treatment or prevention of a disorder or one or more symptoms thereof. In certain embodiments, the term “therapeutic agent” includes a compound and/or an antibody conjugate provided herein. In certain embodiments, a therapeutic agent is an agent which is known to be useful for, or has been or is currently being used for the treatment or prevention of a disorder or one or more symptoms thereof.

The term “therapeutically effective amount” or “effective amount” refers to an amount of an antibody or composition that when administered to a subject is effective to treat a disease or disorder. In some embodiments, a therapeutically effective amount or effective amount refers to an amount of an antibody or composition that when administered to a subject is effective to prevent or ameliorate a disease or the progression of the disease, or result in amelioration of symptoms. A “therapeutically effective amount” can vary depending on, inter alia, the compound, the disease and its severity, and the age, weight, etc., of the subject to be treated.

The term “treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder, or delaying or preventing recurrence of the disease or disorder. In yet another embodiment, “treating” or “treatment” includes the reduction or elimination of either the disease or disorder, or to retard the progression of the disease or disorder or of one or more symptoms of the disease or disorder, or to reduce the severity of the disease or disorder or of one or more symptoms of the disease or disorder.

The term “inhibits growth” (e.g., referring to cells, such as tumor cells) is intended to include any measurable decrease in cell growth (e.g., tumor cell growth) when contacted with an antibody or antibody conjugate, as compared to the growth of the same cells not in contact with the antibody or antibody conjugate. In some embodiments, growth may be inhibited by at least about 30%, 40%, 50%, 60%, 70%, 80%, 90%, 95%, 99%, or 100%. The decrease in cell growth can occur by a variety of mechanisms, including but not limited to antibody internalization, apoptosis, necrosis, and/or effector function-mediated activity.

The terms “prophylactic agent” and “prophylactic agents” as used refer to any agent(s) which can be used in the prevention of a disorder or one or more symptoms thereof. In certain embodiments, the term “prophylactic agent” includes a compound provided herein. In certain other embodiments, the term “prophylactic agent” does not refer to a compound provided herein. For example, a prophylactic agent is an agent which is known to be useful for, or has been or is currently being used to prevent or impede the onset, development, progression, and/or severity of a disorder.

The phrase “prophylactically effective amount” refers to the amount of a therapy (e.g., prophylactic agent) which is sufficient to result in the prevention or reduction of the development, recurrence, or onset of one or more symptoms associated with a disorder, or to enhance or improve the prophylactic effect(s) of another therapy (e.g., another prophylactic agent).

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C₁₋₆alkyl, and the like. In certain embodiments, the alkyl group is selected from the group consisting of methyl, ethyl, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with, for example, one, two, or three groups independently selected from a halogen (e.g., fluoro (F), chloro (Cl), bromo (Br), or iodo (I)), alkyl, —CN, —NO₂, amido, —C(O)—, —C(S)—, ester, carbamate, alkenyl, alkynyl, cycloalkyl, heterocycloalkyl, dialkylamino, haloalkyl, hydroxyl, amino, alkylamino, and alkoxy. In some embodiments, alkyl is unsubstituted. In one embodiment, alkyl is methyl or ethyl.

The term “alkylene,” as used herein, unless otherwise specified, refers to a divalent alkyl group, as defined herein. “Substituted alkylene” refers to an alkylene group substituted as described herein for alkyl. In some embodiments, alkylene is unsubstituted. In one embodiment, alkylene is C₆ alkylene.

“Alkenyl” refers to an olefinically unsaturated hydrocarbon group, in certain embodiments, having up to about eleven carbon atoms or from two to six carbon atoms (e.g., “lower alkenyl”), which can be straight-chained or branched, and having at least one or from one to two sites of olefinic unsaturation. “Substituted alkenyl” refers to an alkenyl group substituted as described herein for alkyl.

“Alkenylene” refers to a divalent alkenyl as defined herein. Lower alkenylene is, for example, C₂-C₆-alkenylene. In one embodiment, alkylene is C₂ alkenylene.

“Alkynyl” refers to acetylenically unsaturated hydrocarbon groups, in certain embodiments, having up to about eleven carbon atoms or from two to six carbon atoms (e.g., “lower alkynyl”), which can be straight-chained or branched, and having at least one or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

“Alkynylene” refers to a divalent alkynyl as defined herein. Lower alkynylene is, for example, C₂-C₆-alkynylene.

“Amino” refers to —NH₂.

The term “aminoalkyl,” as used herein, and unless otherwise specified, refers to the an alkyl group, as defined herein, which is substituted with one or more amino groups. In some embodiments, the aminoalkyl is an alkyl group substituted with one —NH₂ group (e.g., —R′(NH₂) wherein R′ is alkyl as defined herein).

The term “alkylamino,” as used herein, and unless otherwise specified, refers to the group —NHR″ where R″ is, for example, C₁₋₁₀alkyl, C₂₋₁₀ alkenyl, C₂₋₁₀ alkynyl, C₃₋₁₂ carbocycle, 3- to 12-membered heterocycle, C₁₋₁₀ haloalkyl, and the like as defined herein. In certain embodiments, alkylamino is C₁₋₆alkylamino.

The term “dialkylamino,” as used herein, and unless otherwise specified, refers to the group —NR″R″ where each R″ is independently C₁₋₁₀alkyl, as defined herein. In certain embodiments, dialkylamino is, for example, di-C₁₋₆alkylamino, C₂₋₁₀ alkenyl, C₂₋₁₀ alkynyl, C₃₋₁₂ carbocycle, 3- to 12-membered heterocycle, C₁₋₁₀ haloalkyl, and the like.

The term “aryl,” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (see, e.g., Greene, et al., Protective Groups in Organic Synthesis, John Wiley and Sons, Second Edition, 1991); and wherein the aryl in the arylamino and aryloxy substituents are not further substituted.

The term “arylamino,” as used herein, and unless otherwise specified, refers to an —NR′R″ group where R′ is hydrogen or C₁-C₆-alkyl; and R″ is aryl, as defined herein.

The term “arylene,” as used herein, and unless otherwise specified, refers to a divalent aryl group, as defined herein.

The term “aryloxy,” as used herein, and unless otherwise specified, refers to an —OR group where R is aryl, as defined herein.

“Alkarylene” refers to an arylene group, as defined herein, wherein the aryl ring is substituted with one or two alkyl groups. “Substituted alkarylene” refers to an alkarylene, as defined herein, where the arylene group is further substituted, as defined herein for aryl.

“Aralkylene” refers to a —CH₂-arylene-, -arylene-CH₂—, or —CH₂-arylene-CH₂— group, where arylene is as defined herein. “Substituted aralkylene” refers to an aralkylene, as defined herein, where the aralkylene group is substituted, as defined herein for aryl.

“Carboxyl” or “carboxy” refers to —C(O)OH or —COOH.”

“Divalent hydrophilic group;” as used herein, and unless otherwise specified, refers to PEG, for example of the formula

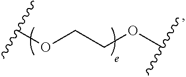

wherein e is an integer between 1 and 50, inclusive; and each [symbol] is the point of attachment to the rest of the formula.

“Polyethylene glycoloxy (PEG)” as used herein, and unless otherwise specified, refers to the monovalent or divalent residue of polyethylene glycol (PEG), for example of the formula

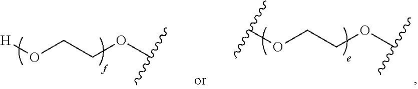

respectively, wherein e or f is an integer between 1 and 50, inclusive; and each [symbol] is the point of attachment to the rest of the formula. In one embodiment, e is an integer between 1 and 10. In one embodiment, e is an integer between 5 and 10. In one embodiment, f is an integer between 11 and 20.

“Methoxypolyethylene glycoloxy” as used herein, and unless otherwise specified, refers to the monovalent residue of poly(ethylene glycol) monomethyl ether (mPEG) and is of the formula

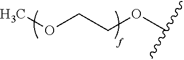

wherein f is an integer between 1 and 50, inclusive; and each [symbol] is the point of attachment to the rest of the formula. In one embodiment, f is an integer between 1 and 20. In one embodiment, f is an integer between 11 and 20.

“Methoxypolyethylene glycoloxy-CH₂CH₂—C(O)—” as used herein, and unless otherwise specified, refers to the monovalent residue of mPEG as described herein further substituted with —CH₂CH₂—C(O)— (mPEG-CH₂CH₂—C(O)—) and is of the formula

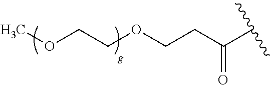

wherein g is an integer between 1 and 50, inclusive; and each [symbol] is the point of attachment to the rest of the formula. In one embodiment, g is an integer between 1 and 20. In one embodiment, g is an integer between 11.

“Polyethylene glycoloxy-CH₂CH₂C(O)—” as used herein, and unless otherwise specified, refers to the monovalent residue of PEG as described herein further substituted with —CH₂CH₂—C(O)— PEG-CH₂CH₂—C(O)—) and is of the formula

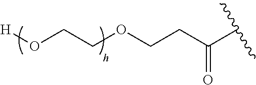

wherein h is an integer between 1 and 50, inclusive; and each [symbol] is the point of attachment to the rest of the formula. In one embodiment, h is an integer between 1 and 20. In one embodiment, h is an integer between 11 and 20.

The term “carbocycle” as used herein, unless otherwise specified, refer to a saturated, unsaturated, or aromatic ring in which all atoms of the ring are carbon. In certain embodiments, the “carbocycle” group may be saturated, and/or bridged, and/or non-bridged, and/or a fused bicyclic group, and/or a spirocyclic bicyclic group. In certain embodiments, the “carbocycle” group includes three to ten carbon atoms (i.e., C₃ to C₁₀ cycloalkyl). In some embodiments, the “carbocycle” has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the “carbocycle” group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl Exemplary “carbocycles” include cyclopentyl, cyclohexyl, cyclohexenyl, adamantyl phenyl, indanyl, and naphthyl. “Carbocycle” includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Non-limiting examples of bridged bicyclic carbocycle groups include, but are not limited to, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, and 2-oxabicyclo[2.2.2]octyl. Non-limiting examples of spirocyclic carbocycle groups include, but are not limited to, spiro[3.3]heptyl, spiro[3.4]octyl, spiro[3.5]nonyl, spiro[3.6]decyl, spiro[4.4]nonyl, spiro[4.5]decyl, spiro[5.5]undecyl, spiro[5.6]dodecyl, and spiro[5.7]tridecyl.

“Carbocyclene” refers to a divalent carbocycle as defined herein.

The term “bicyclic ring system” includes 6-12 (e.g., 8-12 or 9-, 10-, or 11-) membered structures that form two rings, wherein the two rings have at least one atom in common (e.g., two atoms in common). Bicyclic rings can be fused, bridged, or spirocyclic. Bicyclic ring systems include bicycloaliphatics (e.g., bicycloalkyl or bicycloalkenyl), bicycloheteroaliphatics, bicyclic aryls, and bicyclic heteroaryls.

The term “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornenyl, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

The term “spiro bicyclic ring system” refers to a bicyclic heterocyclicalipahtic ring system or bicyclic cycloaliphatic ring system in which 2 or 3 rings are linked together by one common atom. Spiro compounds depicted with overlapping rings indicate that the rings can bond at any vertex. For instance, in the spiro group

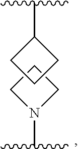

the two rings can bond at any of the three available vertex atoms in either ring. In some embodiments, a spiro bicyclic ring is a 3- to 12-membered spirocyclic bicyclic heterocycle comprising two nitrogen atoms and one oxygen atom. Non-limiting examples of a spirocyclic bicyclic heterocycle include a 10-membered spirocyclic bicyclic heterocycle, a 9-membered spirocyclic bicyclic heterocycle, and a 8-membered spirocyclic bicyclic heterocycle. The 3- to 12-membered spirocyclic bicyclic heterocycle include, but are not limited to, nitrogen (N), oxygen (O), and sulfur (S) atoms, for example two nitrogen atoms and one oxygen atom. For example, a 5-oxa-2,8-diazaspiro[3.5]nonane is a compound in which a 4 membered heterocyclic ring and a 6 membered heterocyclic ring are bonded through a single carbon atom wherein an oxygen atom is in the 6 membered heterocyclic ring.

The term “cycloalkylene,” as used herein refers to a divalent cycloalkyl group, as defined herein. In certain embodiments, the cycloalkylene group is cyclopropylene

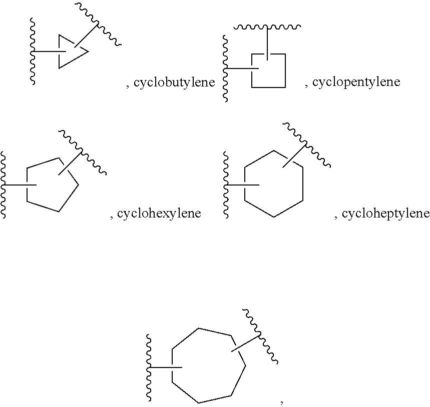

and the like. Lower cycloalkylene refers to a C₃-C₆-cycloalkylene.

The term “cycloalkylalkyl,” as used herein, unless otherwise specified, refers to an alkyl group, as defined herein, substituted with one or two cycloalkyl, as defined herein.

The term “ester,” as used herein, refers to —C(O)OR or —COOR where R is alkyl, as defined herein.

The term “fluorene” as used herein refers to

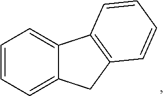 [SMILES: c1ccc2c(c1)Cc1ccccc1-2]

wherein any one or more carbons bearing one or more hydrogens can be substituted with a chemical functional group as described herein.

The term “haloalkyl” refers to an alkyl group, as defined herein, substituted with one or more halogen atoms (e.g., in some embodiments one, two, three, four, or five) which are independently selected.

The term “heteroalkyl” refers to an alkyl, as defined herein, in which one or more carbon atoms are replaced by heteroatoms. As used herein, “heteroalkenyl” refers to an alkenyl, as defined herein, in which one or more carbon atoms are replaced by heteroatoms. As used herein, “heteroalkynyl” refers to an alkynyl, as defined herein, in which one or more carbon atoms are replaced by heteroatoms. Suitable heteroatoms include, but are not limited to, nitrogen (N), oxygen (O), and sulfur (S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one or two heteroatoms, a 5- or 6-membered heteroalkyl may generally comprise one, two, or three heteroatoms, and a 7- to 10-membered heteroalkyl may generally comprise one, two, three, or four heteroatoms.

The term “heteroalkylene,” as used herein, refers to a divalent heteroalkyl, as defined herein. “Substituted heteroalkylene” refers to a divalent heteroalkyl, as defined herein, substituted as described for heteroalkyl.

The term “heterocycle” refers to a saturated, unsaturated or aromatic ring comprising one or more heteroatoms. Exemplary heteroatoms include N, O, Si, P, B, and S atoms where the nitrogen or sulfur atoms may be optionally oxidized, and the nitrogen atoms may be optionally quaternized and the remaining ring atoms of the non-aromatic ring are carbon atoms. A “heterocycle” includes 3- to 10-membered monocyclic rings, 6- to 12-membered bicyclic rings, and 6- to 12-membered bridged rings. In certain embodiments, “heterocycle” is a monovalent, monocyclic, or multicyclic fully-saturated ring system. In certain embodiments, the “heterocycle” group may be unsaturated, and/or bridged, and/or non-bridged, and/or a fused bicyclic group, and/or a spirocyclic bicyclic group. A bicyclic “heterocycle” includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. In certain embodiments, the “heterocycle” group has from three to twenty, from three to fifteen, from three to twelve from three to ten, from three to eight, from four to seven, from four to eleven, or from five to six ring atoms. The “heterocycle” may be attached to a core structure at any heteroatom or carbon atom which results in the creation of a stable compound. In certain embodiments, the “heterocycle” is a monocyclic, bicyclic, tricyclic, or tetracyclic ring system, which may include a fused or bridged or spirocyclic ring system and in which the nitrogen or sulfur atoms may be optionally oxidized, and/or the nitrogen atoms may be optionally quaternized. In some embodiments, “heterocycle” radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, azetidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. Non-limiting examples of bridged heterocycle groups include, but are not limited to, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.0^{3,7}]nonyl. Non-limiting examples of spirocyclic heterocycle groups include, but are not limited to, 2,8-diazaspiro[4.5]decyl; 2,7-diazaspiro[3.5]nonyl; 3,9-diazaspiro[5.5]undecyl; 3-azaspiro[5.5]undecyl; 2-oxa-6-azaspiro[3.4]octyl; 2-oxa-9-azaspiro[5.5]undecyl; 3-oxa-9-azaspiro[5.5]undecyl; 7-azaspiro[3.5]nonyl; 2-azaspiro[3.5]nonyl; 7-oxaspiro[3.5]nonyl; and 2-oxaspiro[3.5]nonyl. In certain embodiments, “heterocycle” may also be optionally substituted as described herein. In certain embodiments, “heterocycle” is substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a “heterocycle” group may comprise one, two, three, or four heteroatoms. Those of skill in the art will recognize that a 4-membered “heterocycle” may generally comprise one or two heteroatoms, a 5- or 6-membered “heterocycle” may generally comprise one, two, or three heteroatoms, and a 7- to 10-membered “heterocycle” may generally comprise one, two, three, or four heteroatoms.

The term “heterocycloalkylene” refers to a divalent heterocycloalkyl as defined herein.

The term “heteroaryl” refers to a monovalent, monocyclic aromatic group and/or multicyclic aromatic group, wherein at least one aromatic ring contains one or more heteroatoms independently selected from oxygen, sulfur, and nitrogen within the ring. Each ring of a heteroaryl group can contain one or two oxygen atoms, one or two sulfur atoms, and/or one to four nitrogen atoms, provided that the total number of heteroatoms in each ring is four or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

The term “heteroarylene” refers to a divalent heteroaryl group, as defined herein. “Substituted heteroarylene” is a heteroarylene substituted as defined for aryl.

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “substantially free of” or “substantially in the absence of” with respect to a composition refers to a composition that includes at least 85 or 90% by weight, in certain embodiments 95%, 98%, 99% or 100% by weight, of the designated enantiomer of that compound. In certain embodiments, in the methods and compounds provided herein, the compounds are substantially free of enantiomers.

Similarly, the term “isolated” with respect to a composition refers to a composition that includes at least 85, 90%, 95%, 98%, and 99% to 100% by weight, of the compound, the remainder comprising other chemical species or enantiomers.

“Solvate” refers to a compound provided herein or a salt thereof that further includes a stoichiometric or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “alkylamino,” “dialkylamino,” “cycloalkyl,” “aryl,” “alkoxy,” “alkoxycarbonyl,” “amino,” “carboxyl,” “heterocyclyl,” “heterocycloalkyl,” “heteroaryl,” “partially saturated heteroaryl,” “carboxyl,” and “amino acid” groups optionally comprise deuterium at one or more positions where hydrogen atoms are present, and wherein the deuterium composition of the atom or atoms is other than the natural isotopic composition.

Also as used herein, “alkyl,” “alkylamino,” “dialkylamino,” “cycloalkyl,” “aryl,” “alkoxy,” “alkoxycarbonyl,” “amino,” “carboxyl,” “heterocyclyl,” “heteroaryl,” “carboxyl” and “amino acid” groups optionally comprise carbon-13 at an amount other than the natural isotopic composition.

In some chemical structures illustrated herein, certain substituents, chemical groups, and atoms are depicted with a curvy/wavy line (e.g., [symbol] ) that intersects a bond or bonds to indicate the atom through which the substituents, chemical groups, and atoms are bonded. For example, in some structures, such as but not limited to,

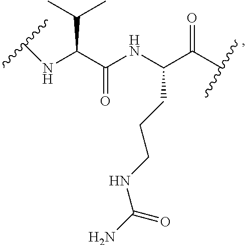 [SMILES: *N[C@H](C(=O)N[C@@H](CCCNC(N)=O)C(*)=O)C(C)C]

the curvy/wavy lines indicates the atoms in the backbone of a conjugate structure to which the illustrated chemical entity is bonded. In some structures, such as but not limited to,

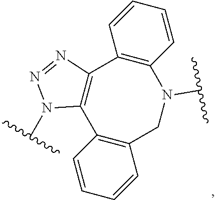 [SMILES: *N1Cc2ccccc2-c2c(nnn2*)-c2ccccc21]

the curvy/wavy lines indicate the atoms in the antibody or antibody fragment as well as the atoms in the backbone of a conjugate or linker-payload structure to which the illustrated chemical entity is bonded.

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

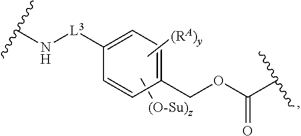

wherein subscripts z and y are integers and in which the positions of substituents —O-Su and R^{A} is described generically, i.e., not directly attached to any vertex of the bond line structure, i.e., specific ring carbon atom, includes the following, non-limiting examples of groups in which the substituent R^{A} and Osu is bonded to a specific ring carbon atom:

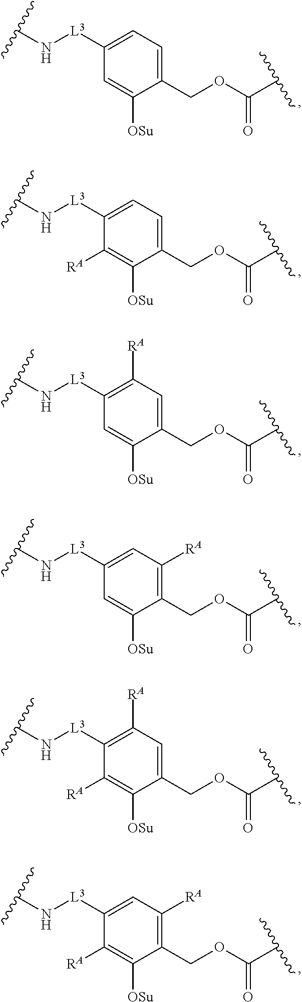

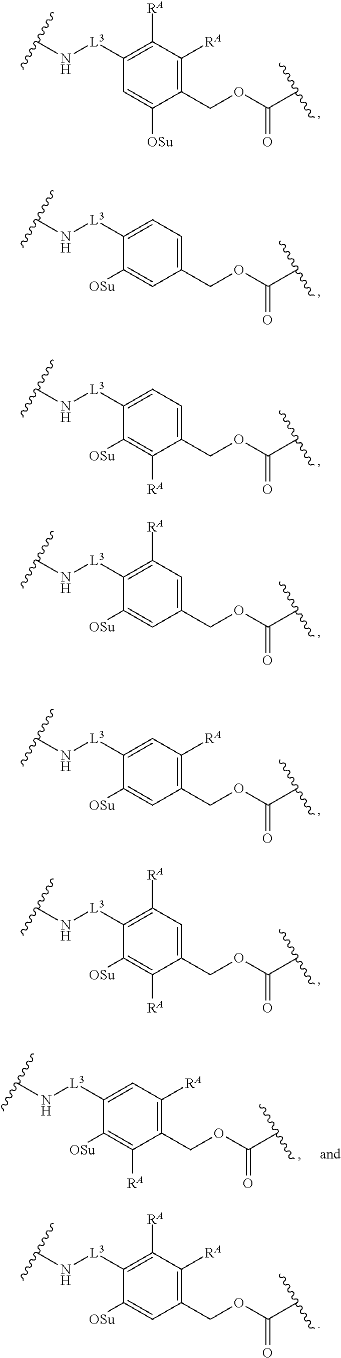

### Conjugates

Provided herein are dual payload immunostimulatory antibody-drug conjugates (iADCs) that comprise an anti-TROP2 antibody or antigen binding fragment thereof, at least one STING agonist via a first linker, and at least one cytotoxic agent. In certain embodiments, the immunostimulatory antibody drug conjugate comprises an anti-TROP2 antibody or antigen binding fragment thereof, wherein the anti-TROP2 antibody or antigen binding fragment thereof is linked site-specifically to at least one STING agonist via first linker, and also linked site-specifically to at least one cytotoxic agent via a second linker. In certain embodiments, the iADCs comprise means for binding TROP2, at least one STING agonist via a first linker, and at least one cytotoxic agent.

Some embodiments of the present invention are shown below:

Embodiment [1]: The conjugate is of Formula I:

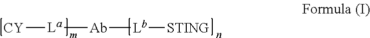

or a pharmaceutically acceptable salt, solvate, stereoisomer, regioisomer, or mixture of regioisomers thereof,
wherein Ab is an anti-TROP2 antibody or antigen-binding fragment thereof,
L^{a} is a first linker;
L^{b} is a second linker;
CY is independently a residue of a cytotoxic agent;
STING is independently a residue of a STING agonist;
n is an integer selected from 1 to 10; and
m is an integer selected from 1 to 10.

Embodiment [1-1]: The conjugate of embodiment [1], wherein the conjugate of Formula (I) is Formula (Ib) or Formula (Ic):

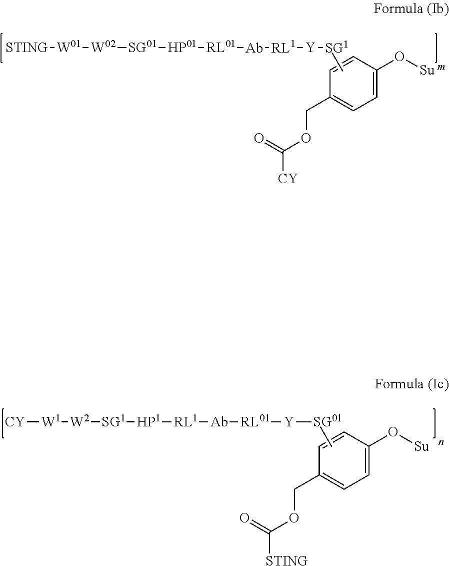

or a pharmaceutically acceptable salt, solvate, tautomer, stereoisomer, regioisomer, or mixture of regioisomers thereof, wherein:
Y is —X¹—C₁₋₆ alkylene-[X¹—C₁₋₆ alkylene]ₐ-[X¹]_{b}—, —X¹—C₂₋₆ alkenylene-[X¹—C₂₋₆ alkenylene]ₐ-[X¹]_{b}—, or —X¹—C₂₋₆ alkynylene-[X¹—C₂₋₆ alkynylene]ₐ-[X¹]_{b}—, wherein at least one C₁₋₆ alkylene, C₂₋₆ alkenylene, or C₂₋₆ alkynylene in Y is substituted with one or more substituents selected from R⁷⁰; and —[X¹]_{b}— is attached to RL¹ or RL⁰¹; and
wherein the C₁₋₆ alkylene, C₂₋₆ alkenylene, or C₂₋₆ alkynylene in Y is optionally substituted with one or more substituents selected from R⁷¹;
R⁷⁰ is —C₁₋₆ alkylene-X²—[C₁₋₆ alkylene]_{c}-HP², —C₂₋₆ alkenylene-X²—[C₂₋₆ alkenylene]_{c}-HP², or —C₂₋₆ alkynylene-X²—[C₂₋₆ alkynylene]_{c}-HP², wherein each C₁₋₆ alkylene, C₂₋₆ alkenylene, or C₂₋₆ alkynylene of R⁷⁰ is optionally substituted with one or more substituents selected from halogen, —CN, —NO₂, —OH, —NR⁶⁰R⁶¹, —C(O)NR⁶⁰R⁶¹, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
R⁷¹ is independently selected from halogen, —CN, —NO₂, —OH, —NR⁶⁰R⁶¹, —C(O)NR⁶⁰R⁶¹, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
X¹ and X² are independently selected from —N(R⁶⁰)—, —C(O)—, and —N(R⁶⁰)C(O)—;
a is an integer selected from 0, 1, 2, and 3;
b is an integer selected from 0 and 1;
c is an integer selected from 0 and 1; and
Su is a hexose form of a monosaccharide.

Embodiment [1-2]: The conjugate of embodiment [1-1], wherein the conjugate of Formula (Ib) is Formula (Ib-1):

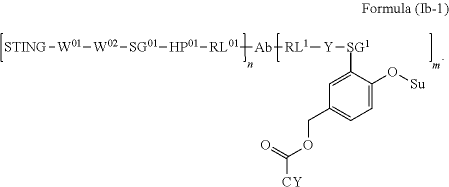

Embodiment [1-3]: The conjugate of embodiment [1-1], wherein the conjugate of Formula (Ib) is Formula (Ib-2):

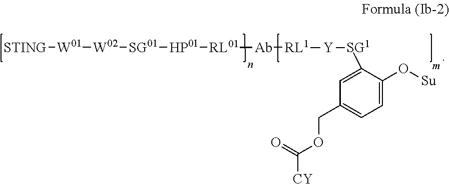

Embodiment [1-4]: The conjugate of embodiment [1-1], wherein the conjugate of Formula (Ic) is Formula (Ic-1):

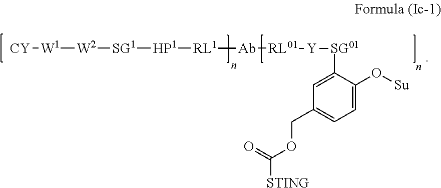

Embodiment [1-5]: The conjugate of embodiment [1-1], wherein the conjugate of Formula (Ic) is Formula (Ic-2):

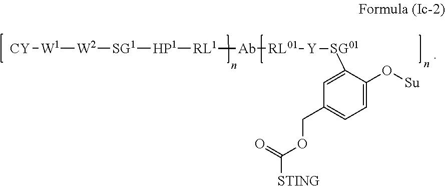

Embodiment [2]: The conjugate of any one of embodiments [1]-[1-5], wherein the conjugate is of Formula (I), (Ib), (Ic), (Ib-1), (Ib-2), (Ic-1), or (Ic-2), wherein STING is independently a residue of a compound of Formula (S-I) or (S-II):

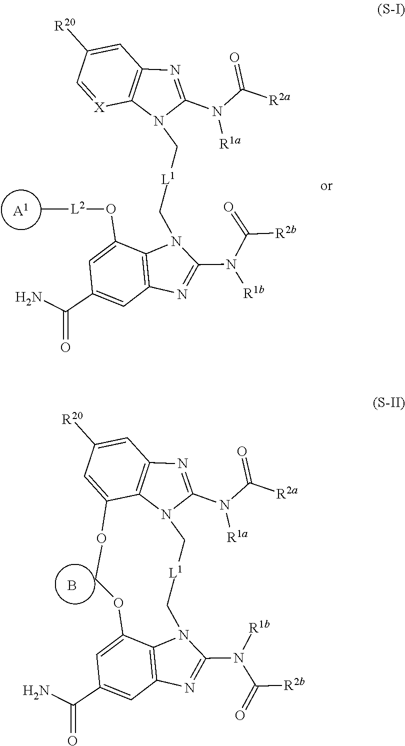

or a tautomer or stereoisomer thereof,
wherein:
X is selected from N and CR³;
R²⁰ is selected from hydrogen and —CON(R^{3a})(R^{3b});
R^{1a}, R^{1b}, R^{3a} and R^{3b} are independently selected from hydrogen and optionally substituted C₁₋₆ alkyl, wherein the C₁₋₆ alkyl is optionally substituted with one or more R⁵⁰;
R^{2a} and R^{2b} are independently selected from: (a) optionally substituted C₁₋₆ alkyl, wherein the C₁₋₆ alkyl is optionally substituted with one or more R⁵¹ and (b) optionally substituted C₃₋₁₂ carbocycle or optionally substituted 3- to 12-membered heterocycle, each of which is optionally substituted with one or more R⁵³,
or R^{1a} and R^{2a} are joined together with the atoms to which they are attached to form an optionally substituted 3- to 12-membered heterocycle, wherein the heterocycle is optionally substituted with one or more R⁵³;
or R^{1b} and R^{2b} are joined together with the atoms to which they are attached to form an optionally substituted 3- to 12-membered heterocycle, wherein the heterocycle is optionally substituted with one or more R⁵³;
R³ is hydrogen, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, or —CN;
L¹ is selected from a bond, —C₁₋₁₀alkylene-, —C₂₋₁₀alkenylene-, —C₂₋₁₀alkynylene-, —C₁₋₆alkylene-O—C₁₋₆alkylene-, —C₁₋₆alkylene-NH—C₁₋₆alkylene-, C₃₋₆carbocyclene, and —C₁₋₆alkylene-(C₃₋₆carbocyclene)-C₁₋₆alkylene-, wherein —C₁₋₁₀ alkylene-, —C₂₋₁₀ alkenylene-, —C₂₋₁₀ alkynylene-, C₃₋₆ carbocyclene, and each C₁₋₆ alkylene of —C₁₋₆alkylene-O—C₁₋₆alkylene-, —C₁₋₆alkylene-NH—C₁₋₆alkylene-, and —C₁₋₆alkylene-(C₃₋₆carbocyclene)-C₁₋₆alkylene- are optionally substituted with one or more R⁵⁰;
L² is optionally substituted —C₁₋₆ alkylene- or —C₁₋₆alkylene-O—, wherein the —C₁₋₆ alkylene- or the C₁₋₆ alkylene of —C₁₋₆ alkylene-O— is optionally substituted with one or more R⁵⁰ and wherein the —C₁₋₆ alkylene-O— is attached to the A ring, or L^{b} if the A ring is absent, via the oxygen atom;
Ring A is absent; an optionally substituted bridged, fused, or spirocyclic bicyclic heterocycle comprising at least one N atom and at least one O atom, wherein the heterocycle is optionally substituted with one or more R⁵³; or, a 3- to 12-membered heterocycle substituted with R⁴;
Ring B is an optionally substituted C₃₋₁₂ carbocycle or an optionally substituted 3- to 12-membered heterocycle, wherein the C₃₋₁₂ carbocycle and 3- to 12-membered heterocycle are optionally substituted with one or more R⁵²;
R⁴ is an optionally substituted 3- to 12-membered heterocycle optionally comprising at least one NR⁵ and, wherein the heterocycle is optionally substituted with one or more R⁵³;
R⁵ is independently selected from hydrogen, R⁶, —C(O)—C₁₋₆alkyl, —C(O)-heteroC₁₋₆alkyl, C₁₋₆ alkyl, and heteroC₁₋₆alkyl wherein the C₁₋₆ alkyl, either alone or part of another group, is optionally substituted with one or more R⁵⁰;
R⁶ is independently an amino acid residue;
R³⁰ is independently selected from hydrogen, C₁₋₁₀ alkyl, C₂₋₁₀ alkenyl, C₂₋₁₀ alkynyl, C₃₋₁₂ carbocycle, and 3- to 12-membered heterocycle, each of which, except hydrogen, is optionally substituted with one or more R⁵⁵;
R⁵⁰ is independently selected from halogen, —OR⁶⁰, —SR⁶⁰, —C(O)NR⁶⁰R⁶¹, —N(R⁶⁰)C(O)R⁶⁰, —N(R⁶⁰)C(O)NR⁶⁰R⁶¹, —NR⁶⁰R⁶¹, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;
R⁵¹ is independently selected from halogen, —OR⁶⁰, —SR⁶⁰, —C(O)NR⁶⁰R⁶¹, —N(R⁶⁰)C(O)R⁶⁰, —N(R⁶⁰)C(O)NR⁶⁰R⁶¹, —NR⁶⁰R⁶¹, —C(O)R⁶⁰, —C(O)OR⁶⁰, —OC(O)R⁶⁰, —NO₂, ═O, ═S, ═N(R⁶⁰), —CN, optionally substituted C₃₋₁₂ carbocycle, and optionally substituted 3- to 12-membered heterocycle, wherein the C₃₋₁₂ carbocycle and 3- to 12-membered heterocycle are optionally substituted with one or more R⁵²;
R⁵² is independently selected from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;
R⁵³ is independently selected from halogen, —OR⁶⁰, —SR⁶⁰, —C(O)NR⁶⁰R⁶¹, —N(R⁶⁰)C(O)R⁶⁰, —N(R⁶⁰)C(O)NR⁶⁰R⁶¹, —NR⁶⁰R⁶¹, —C(O)R⁶⁰, —C(O)OR⁶⁰, —OC(O)R⁶⁰, —NO₂, ═O, ═S, ═N(R⁶⁰), —CN, optionally substituted C₁₋₆ alkyl, optionally substituted C₂₋₆ alkenyl, optionally substituted C₂₋₆ alkynyl, optionally substituted C₃-12 carbocycle, and optionally substituted 3- to 12-membered heterocycle, wherein the C₁₋₆ alkyl, C₂₋₆ alkenyl, and C₂₋₆ alkynyl are optionally substituted with one or more R⁵⁴ and the C₃₋₁₂ carbocycle and 3- to 12-membered heterocycle are optionally substituted with one or more R⁵²;
R⁵⁴ is independently selected from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;
R⁵⁵ is independently selected from halogen, —CN, —NO₂, —OH, —NR⁶⁰R⁶¹, —C(O)NR⁶⁰R⁶¹, ═O, ═S, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl; and
R⁶⁰ and R⁶¹ are independently selected from hydrogen, C₁₋₁₀ alkyl optionally substituted with NH₂ or NHC₁₋₆ alkyl, C₂₋₁₀ alkenyl, C₂₋₁₀ alkynyl, C₃₋₁₂ carbocycle, and 3- to 12-membered heterocycle;
wherein when Ring A is absent, L² is —C₁₋₆ alkylene-O—;
wherein the residue of Formula (S-I) is attached to L^{b} via an amino group of Ring A, the oxygen of —C₁₋₆ alkylene-O— of L², an amino group of R⁵⁴, or an amino group of R⁴, and the residue of Formula (S-II) is attached to L^{b} via an amino group of Ring B.

Embodiment [2-1]: The conjugate of any one of embodiments [1] to [2], wherein the conjugate is of Formula (I), (Ib), (Ic), (Ib-1), (Ib-2), (Ic-1), or (Ic-2), wherein STING is independently a residue of a compound of Formula (S-I):

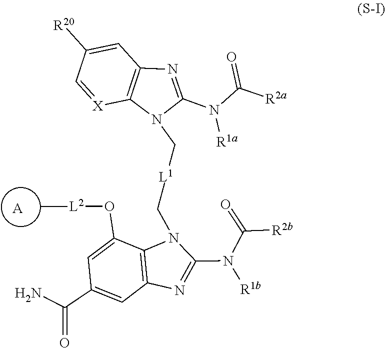

or a tautomer or stereoisomer thereof,
wherein:
X is selected from N and CR³;
R²⁰ is selected from hydrogen and —CON(R^{3a})(R^{3b});
R^{1a}, R^{1b}, R^{3a} and R^{3b} are hydrogen;
R^{2a} and R^{2b} are independently selected from: optionally substituted 3- to 12-membered heterocycle, each of which is optionally substituted with one or more R⁵³;
R³ is hydrogen or —OR³⁰;
L¹ is selected from a bond and —C₂₋₁₀ alkenylene-;
L² is optionally substituted —C₁₋₆ alkylene-;
Ring A is absent; an optionally substituted spirocyclic bicyclic heterocycle comprising at least one N atom and at least one 0 atom, wherein the heterocycle is optionally substituted with one or more R⁵³; or, a 3- to 12-membered heterocycle substituted with R⁴;
R⁴ is an optionally substituted 3- to 12-membered heterocycle optionally comprising at least one NR⁵ and wherein the heterocycle is optionally substituted with one or more R⁵³;
R⁵ is independently selected from hydrogen, R⁶, —C(O)—C₁₋₆alkyl, —C(O)-heteroC₁₋₆alkyl, C₁₋₆ alkyl, and heteroC₁₋₆alkyl wherein the C₁₋₆ alkyl, either alone or part of another group, is optionally substituted with one or more R⁵⁰;
R⁶ is independently an amino acid residue;
R³⁰ is independently selected from hydrogen and C₁₋₁₀ alkyl;
R⁵⁰ is independently selected from halogen, —OR⁶⁰, —SR⁶⁰, —C(O)NR⁶⁰R⁶¹, —N(R⁶⁰)C(O)R⁶⁰, —N(R⁶⁰)C(O)NR⁶⁰R⁶¹, —NR⁶⁰R⁶¹, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;
R⁵³ is independently selected from optionally substituted 3- to 12-membered heterocycle, wherein the 3- to 12-membered heterocycle is optionally substituted with one or more R⁵²; and
wherein when Ring A is absent, L² is —C₁₋₆ alkylene-O—;
wherein the residue of Formula (S-I) is attached to L^{b} via an amino group of Ring A, the oxygen of —C₁₋₆ alkylene-O— of L², an amino group of R⁵⁴, or an amino group of R⁴, and the residue of Formula (S-II) is attached to L^{b} via an amino group of Ring B.

Embodiment [2-2]: The conjugate of embodiment [2-1], wherein the conjugate is of Formula (I), (Ib), (Ic), (Ib-1), (Ib-2), (Ic-1), or (Ic-2), wherein STING is independently a residue of a compound of Formula (S-I), or a tautomer or stereoisomer thereof;
wherein Ring A is selected from

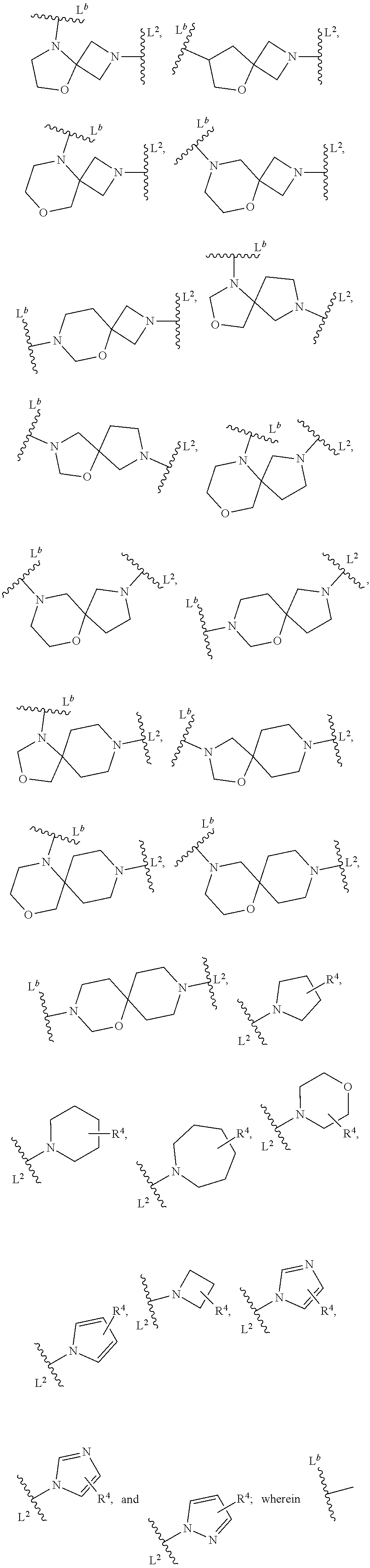

is the point of attachment to L^{b} and [symbol] is the point of attachment to L²; and
R⁴ is

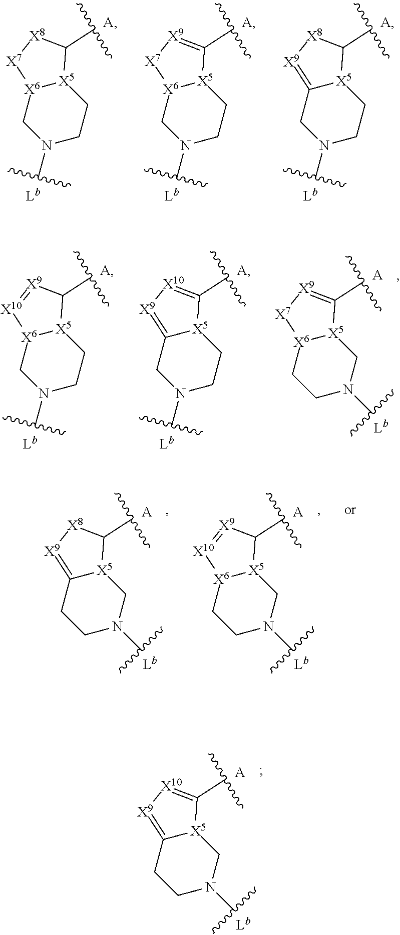

wherein X⁵, X⁶, X⁹, X¹⁰ are independently selected from N and CR¹³;
X⁷ and X⁸ are independently selected from NH, O, and CHR¹³;
R¹³ is independently hydrogen or C₁₋₆ alkyl;
wherein if four of any one of X⁵-X¹⁰ are present, at least one is CR¹³ or CHR¹³; and [symbol] is the point of attachment to Ring A and [symbol] is the point of attachment to L^{b}.

Embodiment [2-3]: The conjugate of embodiment [2-1], wherein the conjugate is of Formula (I), (Ib), (Ic), (Ib-1), (Ib-2), (Ic-1), or (Ic-2), wherein STING is independently a residue of a compound of Formula (S-I), or a tautomer or stereoisomer thereof,
wherein Ring A is

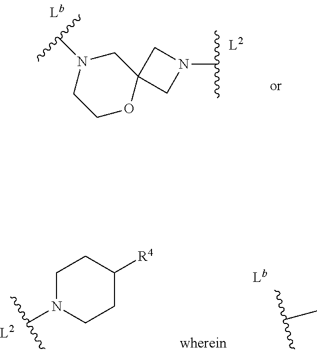

is the point of attachment to L^{b} and [symbol] is the point of attachment to L²; and
R⁴ is

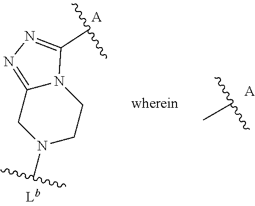

is the point of attachment to ring A and [symbol] is the point of attachment to L^{b}.

Embodiment [3]: The conjugate of any one of embodiments [1]-[2-3], wherein the conjugate is of Formula (I), (Ib), (Ic), (Ib-1), (Ib-2), (Ic-1), or (Ic-2), wherein CY is a residue of a compound selected from the group consisting of an alkylating agent, a DNA-crosslinking agent, an anti-tumor antibiotic, an anti-metabolite, an anti-mitotic agent, a histone-deacetylase (HDAC) inhibitor, a telomerase inhibitor, an immunogenic cell death agent, a tubulin inhibitor, and a topoisomerase inhibitor.

Embodiment [4]: The conjugate of embodiment [3], wherein the conjugate is of Formula (I), (Ib), (Ic), (Ib-1), (Ib-2), (Ic-1), or (Ic-2), wherein CY is a topoisomerase I inhibitor selected from the group consisting of camptothecin, irinotecan, SN-38, topotecan, and exatecan.

Embodiment [5]: The conjugate of embodiment [4], wherein the conjugate is of Formula (I), (Ib), (Ic), (Ib-1), (Ib-2), (Ic-1), or (Ic-2), wherein CY is exatecan.

Embodiment [6]: The conjugate of any one of embodiments [1] to [5], wherein the conjugate is of Formula (I), (Ib), (Ic), (Ib-1), (Ib-2), (Ic-1), or (Ic-2), wherein L^{a} and L^{b} independently comprise a protease cleavable linker, an enzyme cleavable linker, a pH-sensitive linker, or a non-cleavable linker.

Embodiment [7]: The conjugate of any one of embodiments [1] to [6], wherein the conjugate is of Formula (I), (Ib), (Ic), (Ib-1), (Ib-2), (Ic-1), or (Ic-2), wherein
L^{a} is -W¹-W²-SG¹-HP¹-RL¹-;
L^{b} is -RL⁰¹-HP⁰¹-SG⁰¹-W⁰²-W⁰¹-; and
the antibody conjugate of Formula (I) is represented by Formula (Ia):

[CY-W¹—W²-SG¹-HP¹-RL¹-]ₘ-Ab-[-RL⁰¹-HP⁰¹-SG⁰¹-W⁰²-W⁰¹-STING]ₙ (Ia)
or a pharmaceutically acceptable salt, solvate, tautomer, stereoisomer, regioisomer, or mixture of regioisomers thereof, wherein:
Ab is the anti-TROP2 antibody or antigen-binding fragment thereof,
CY is the residue of the cytotoxic agent;
STING is the residue of the STING agonist;
SG¹ and SG⁰¹ are independently, at each occurrence, absent or a divalent spacer group;
W¹ and W⁰¹ are independently, at each occurrence, absent,

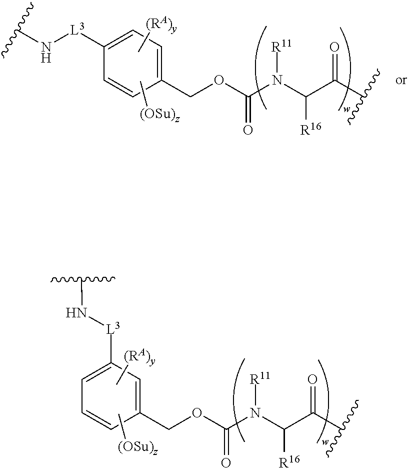

wherein the —NH— is bound to W² or W⁰² and each [symbol] indicates a point of attachment to the rest of the formula;
L³ is independently a bond or an optionally substituted C₁₋₆ alkylene wherein the C₁₋₆alkylene is optionally substituted with one, two, or three substituents selected from halogen, C₁₋₆ alkyl, haloC₁₋₆ alkyl, hydroxyl, amino, C₁₋₆ alkylamino, and C₁₋₆ alkoxy;
R¹⁶ is independently a residue of an amino acid sidechain;
R¹¹ is independently hydrogen or C₁₋₆ alkyl;
w is an integer selected from 0, 1, and 2;
W² and W⁰² are independently, at each occurrence, absent, an amino acid residue, or a peptide residue wherein the amino acid residue or peptide residue is optionally substituted with a HP² group;
HP¹ and HP⁰¹ are independently, at each occurrence, absent or a divalent hydrophilic group;
HP², when present, is a monovalent hydrophilic group;
R^{A} is independently, at each occurrence, optionally substituted C₁₋₆ alkyl wherein the C₁₋₆ alkyl is optionally substituted with one, two, or three substituents selected from halogen, haloC₁₋₆ alkyl, hydroxyl, amino, alkylC₁₋₆ amino, and C₁₋₆ alkoxy;
O-Su is independently, at each occurrence, a release trigger group;
RL¹ and RL⁰¹ are independently, at each occurrence, a reactive linker;
y is an integer independently selected from 0, 1, and 2; and
z is an integer independently selected from 0 and 1.

Embodiment [8]: The conjugate of any one of embodiments [1] to [7], wherein the conjugate is of Formula (I), (Ib), (Ic), (Ib-1), (Ib-2), (Ic-1), or (Ic-2), wherein R^{1a} and Rib are both hydrogen.

Embodiment [9]: The conjugate of any one of embodiments [1] to [8], wherein the conjugate is of Formula (I), (Ib), (Ic), (Ib-1), (Ib-2), (Ic-1), or (Ic-2), wherein R^{2a} and R^{2b} are independently an optionally substituted 3- to 12-membered heterocycle comprising at least one N atom.

Embodiment [10]: The conjugate of embodiment [8] or [9], wherein the conjugate is of Formula (I), (Ib), (Ic), (Ib-1), (Ib-2), (Ic-1), or (Ic-2), wherein R^{2a} and R^{2b} are independently selected from

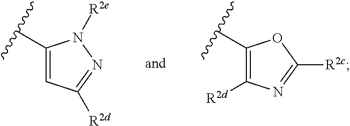

wherein R^{2c} and R^{2d} are independently selected from hydrogen, C₁₋₆ alkyl optionally substituted with one or more R⁵⁴, halo, and 3-12 membered heterocycle;
R^{2e} is hydrogen or C₁₋₆ alkyl; and
[symbol] is the point of attachment to the rest of the formula.

Embodiment [11]: The conjugate of any one of embodiments [2] to [10], wherein the conjugate is of Formula (I), (Ib), (Ic), (Ib-1), (Ib-2), (Ic-1), or (Ic-2)], wherein the STING is a residue of a compound of Formula (S-I) wherein Ring A is an optionally substituted 3- to 12-membered N-linked spirocyclic bicyclic heterocycle comprising at least one nitrogen atom, including the nitrogen bound to L^{b}, and at least one oxygen atom; or a 3- to 12-membered N-linked monocyclic heterocycle substituted with R⁴.

Embodiment [11-1]: The conjugate of any one of embodiments [2]-[11], wherein the conjugate is of Formula (I), (Ib), (Ic), (Ib-1), (Ib-2), (Ic-1), or (Ic-2), wherein the STING is a residue of a compound of Formula (S-I) wherein
a)

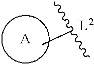

is selected from

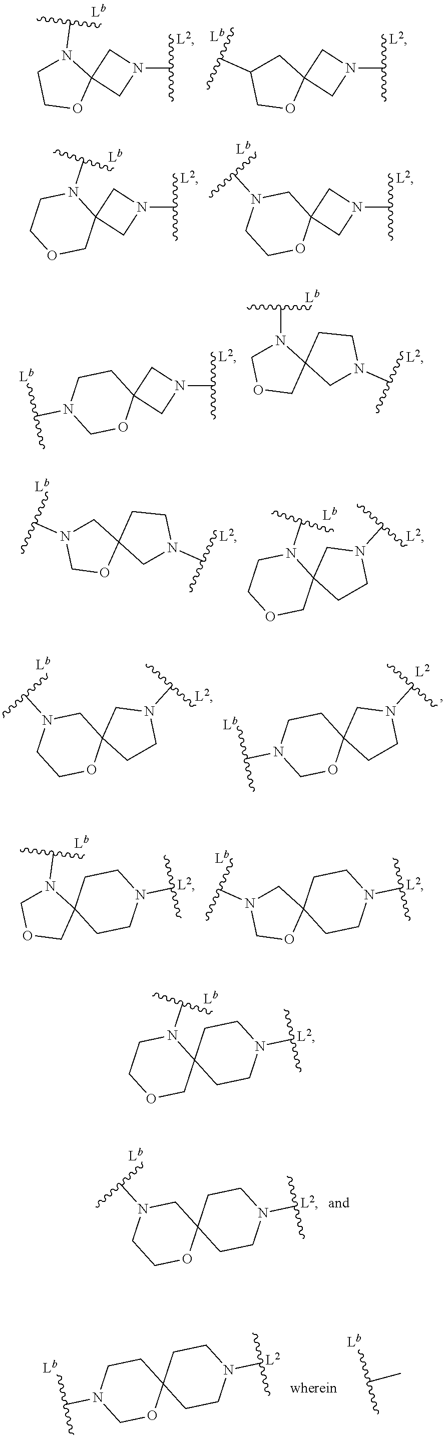

is the point of attachment to L^{b} and [symbol] is the point of attachment to L²; or
b) Ring A is selected from

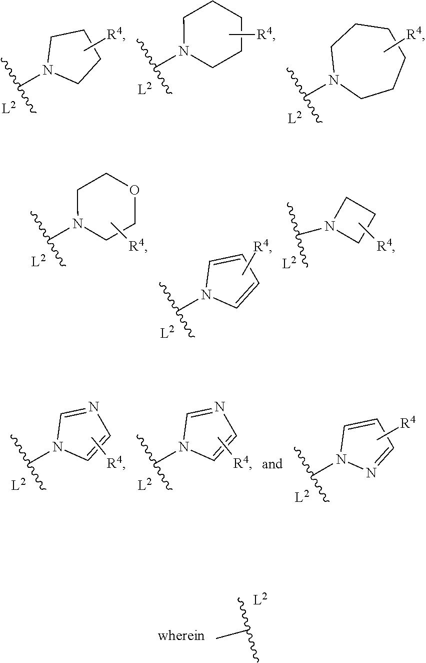

is the point of attachment to L².

Embodiment [11-2]: The conjugate of any one of embodiments [2]-[11], wherein the conjugate is of Formula (I), (Ib), (Ic), (Ib-1), (Ib-2), (Ic-1), or (Ic-2), wherein the STING is a residue of a compound of Formula (S-I) wherein
Ring A

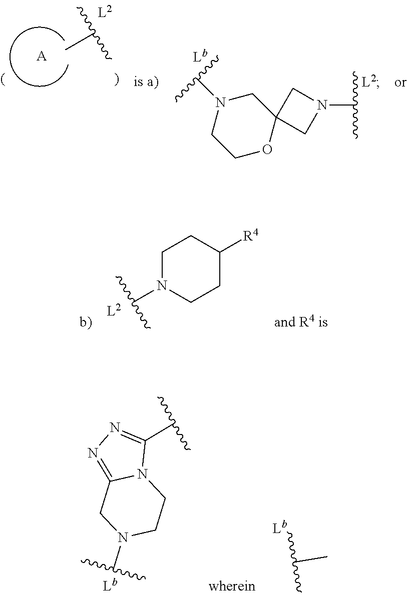

is the point of attachment to L^{b} and [symbol] is the point of attachment to L².

Embodiment [12]: The conjugate of any one of embodiments [2]-[11], wherein the conjugate is of Formula (I), (Ib), (Ic), (Ib-1), (Ib-2), (Ic-1), or (Ic-2) wherein

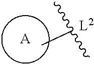

is selected from

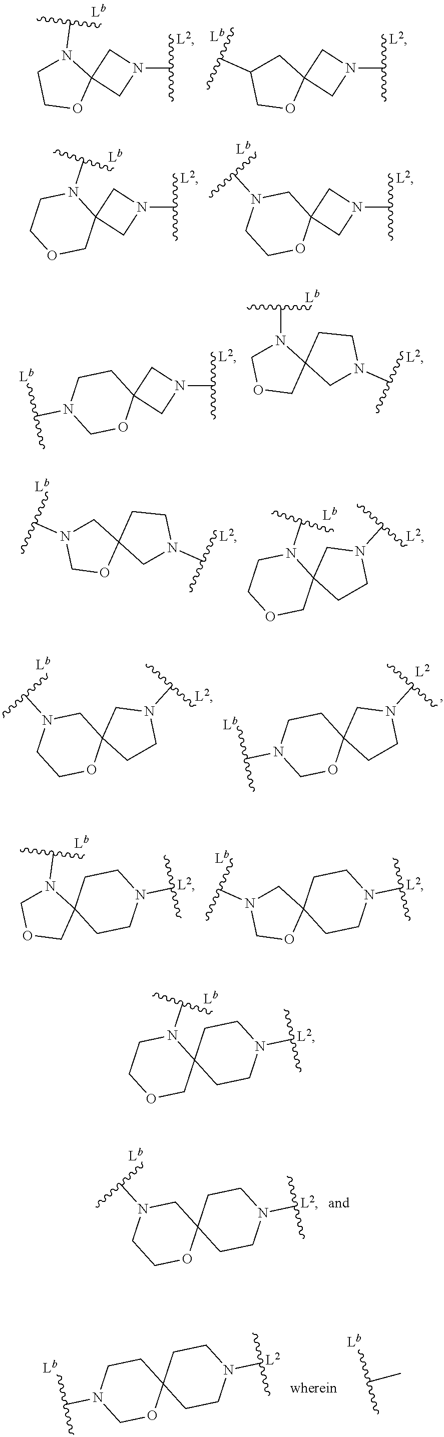

is the point of attachment to L^{b} and [symbol] is the point of attachment to L².

Embodiment [13]: The conjugate of embodiment [12], wherein the conjugate is of Formula (I), (Ib), (Ic), (Ib-1), (Ib-2), (Ic-1), or (Ic-2), wherein

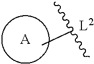

is

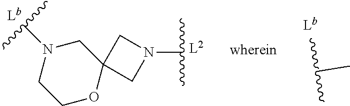

is the point of attachment to L^{b} and [symbol] is the point of attachment to L².

Embodiment [14]: The conjugate of any one of embodiments [2] to [13], wherein the conjugate is of Formula (I), (Ib), (Ic), (Ib-1), (Ib-2), (Ic-1), or (Ic-2), wherein Ring A is an N-linked mono 3- to 12-membered heterocycle substituted with R⁴.

Embodiment [15]: The conjugate of any one of embodiments [2] to [11], wherein the conjugate is of Formula (I), (Ib), (Ic), (Ib-1), (Ib-2), (Ic-1), or (Ic-2), wherein Ring A is selected from

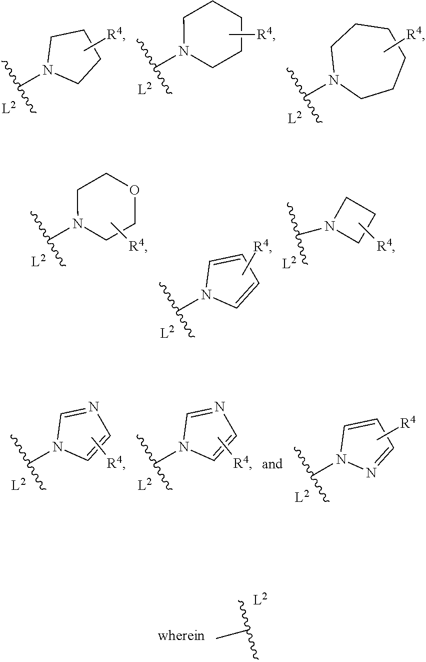

is the point of attachment to L².

Embodiment [16]: The conjugate of embodiment [14] or [15], wherein the conjugate is of Formula (I), (Ib), (Ic), (Ib-1), (Ib-2), (Ic-1), or (Ic-2), wherein Ring A is

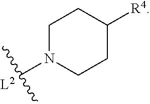

Embodiment [17]: The conjugate of any one of embodiments [2] to [11], wherein the conjugate is of Formula (I), (Ib), (Ic), (Ib-1), (Ib-2), (Ic-1), or (Ic-2) wherein R⁴ is a 5-5 fused ring system, a 5-6 fused ring system, a 6-6 fused ring system, or a 5-7 fused ring system comprising 1, 2, 3, or 4 heteroatoms selected from N, S, and O wherein at least one heteroatom is N bound to L^{b}.

Embodiment [18]: The conjugate of any one of embodiments [2] to [11] or [16], wherein the conjugate is of Formula (I), (Ib), (Ic), (Ib-1), (Ib-2), (Ic-1), or (Ic-2), wherein R⁴ is

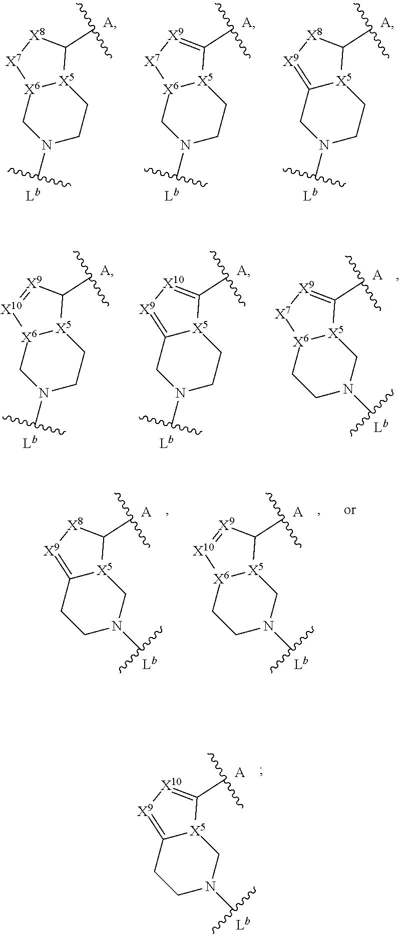

wherein X⁵, X⁶, X⁹, X¹⁰ are independently selected from N and CR¹³;
X⁷ and X⁸ are independently selected from NH, O, and CHR¹³;
R¹³ is independently hydrogen or C₁₋₆ alkyl;
wherein if four of any one of X⁵-X¹⁰ are present, at least one is CR¹³ or CHR¹³; and
[symbol] is the point of attachment to ring A and [symbol] is the point of attachment to L^{b}.

Embodiment [19]: The conjugate of embodiment [18], wherein the conjugate is of Formula (I), (Ib), (Ic), (Ib-1), (Ib-2), (Ic-1), or (Ic-2), wherein R⁴ is

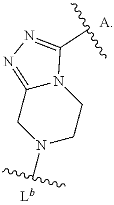

Embodiment [20]: The conjugate of any one of embodiments [1] to [19], wherein the conjugate is of any one of Formula (I), (Ib), (Ic), (Ib-1), (Ib-2), (Ic-1), or (Ic-2), wherein Ring B is an optionally substituted 3- to 12-membered heterocycle.

Embodiment [21]: The conjugate of any one of embodiments [1] to [20], wherein the conjugate is of Formula (I), (Ib), (Ic), (Ib-1), (Ib-2), (Ic-1), or (Ic-2), wherein L² is —C₁₋₆ alkylene-.

Embodiment [22]: The conjugate of any one of embodiments [2] to [21], wherein the conjugate is of Formula (I), (Ib), (Ic), (Ib-1), (Ib-2), (Ic-1), or (Ic-2), wherein each
W¹ is

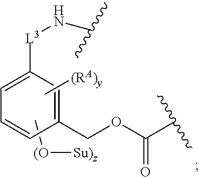

W⁰¹ is independently absent,

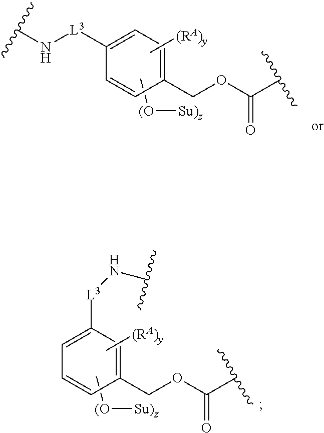

L³ is bond or —CH₂—;
y is 0 or 1; and
z is 0 or 1.

Embodiment [22-1]: The conjugate of any one of embodiments [2]-[21], wherein the conjugate is of Formula (I), (Ib), (Ic), (Ib-1), (Ib-2), (Ic-1), or (Ic-2), wherein
each W¹ is

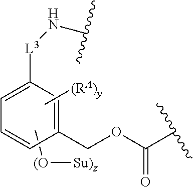

wherein L³ is —CH₂—, y is 0, and z is 1; and
W⁰¹ is independently absent;

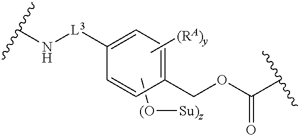

wherein L³ is bond, y is 0, and z is 0; or,

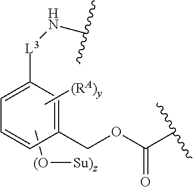

wherein L³ is —CH₂—, y is 0, and z is 1.

Embodiment [23]: The conjugate of embodiment [22], wherein the conjugate is of Formula (I), (Ib), (Ic), (Ib-1), (Ib-2), (Ic-1), or (Ic-2), wherein Su is

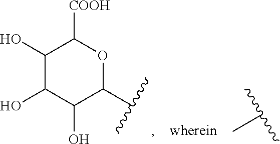

represents attachment to the remainder of the compound.

Embodiment [24]: The conjugate of embodiment [22] to [23], wherein the conjugate is of Formula (I), (Ib), (Ic), (Ib-1), (Ib-2), (Ic-1), or (Ic-2) wherein Su is

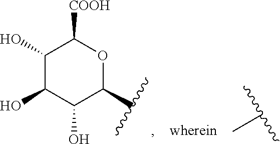

represents attachment to the remainder of the compound.

Embodiment [25]: The conjugate of any one of embodiment [2] to [24], wherein the conjugate is of Formula (I), (Ib), (Ic), (Ib-1), (Ib-2), (Ic-1), or (Ic-2), wherein
each W² and W⁰² are independently absent or a group selected from —(C(O)CHR¹⁰NR¹¹)ₒ—, —(C(O)CH₂CHR¹⁰NR¹¹)ₚ—, —(C(O)CHR¹⁰CH₂NR¹¹)ₚ—, —(C(O)CH₂CHR¹⁰NR¹¹)ₚ—(C(O)CHR¹⁰NR¹¹)ₒ—, and —(C(O)CHR¹⁰CH₂NR¹¹)ₚ—(C(O)CHR¹⁰NR¹¹)ₒ—;
wherein R¹⁰ is independently an amino acid sidechain residue optionally substituted with HP²;
R¹¹ is independently hydrogen or C₁₋₆ alkyl;
and p are independently an integer between 1 and 10, inclusive; and
the —C(O)— of W² and W⁰² are bound to W¹.

Embodiment [26-1]: The conjugate of embodiment [25], wherein the conjugate is of Formula (I), (Ib), (Ic), (Ib-1), (Ib-2), (Ic-1), or (Ic-2), wherein
each W² and W⁰² is independently absent or —(C(O)CH₂CHR¹⁰NR¹¹)ₚ—(C(O)CHR¹⁰NR¹¹)ₒ— wherein
R¹⁰ is an amino acid sidechain residue independently selected from valine, citrulline, alanine, glycine, leucine, asparagine, lysine and 2,3-diaminopropionate and wherein the amino acid sidechain is optionally substituted with HP²;
HP² is independently polyethylene glycoloxy-CH₂CH₂C(O)— (PEG-CH₂CH₂C(O)—); methoxypolyethylene glycoloxy-CH₂CH₂C(O)— (mPEG-CH₂CH₂C(O)—); poly(propylene glycoloxy-CH₂CH₂C(O)—) (PPG-CH₂CH₂C(O)—); poly(oxyethylated polyol)oxy-CH₂CH₂C(O)—; poly(olefinic alcohol)oxy-CH₂CH₂C(O)—; poly(saccharides)-CH₂CH₂C(O)—; poly(vinyl alcohol)oxy-CH₂CH₂C(O)—; or a combination thereof,
R¹¹ is hydrogen;
is independently an integer between 1 and 4, inclusive; and
p is 1.

Embodiment [26]: The conjugate of embodiment [25], wherein the conjugate is of Formula (I), (Ib), (Ic), (Ib-1), (Ib-2), (Ic-1), or (Ic-2), wherein
each W² and W⁰² is independently absent or —(C(O)CH₂CHR¹⁰NR¹¹)ₚ—(C(O)CHR¹⁰NR¹¹)ₒ— wherein
R¹⁰ is an amino acid sidechain residue independently selected from valine, citrulline, alanine, glycine, and 2,3-diaminopropionate and wherein the amino acid sidechain is optionally substituted with HP²;
HP² is independently polyethylene glycoloxy-CH₂CH₂C(O)— (PEG-CH₂CH₂C(O)—); methoxypolyethylene glycoloxy-CH₂CH₂C(O)— (mPEG-CH₂CH₂C(O)—); poly(propylene glycoloxy-CH₂CH₂C(O)—) (PPG-CH₂CH₂C(O)—); poly(oxyethylated polyol)oxy-CH₂CH₂C(O)—; poly(olefinic alcohol)oxy-CH₂CH₂C(O)—; poly(saccharides)-CH₂CH₂C(O)—; poly(vinyl alcohol)oxy-CH₂CH₂C(O)—; or a combination thereof,
R¹¹ is hydrogen;
is independently an integer between 1 and 4, inclusive; and
p is 1.

Embodiment [27]: The conjugate of embodiment [26], wherein the conjugate is of Formula (I), (Ib), (Ic), (Ib-1), (Ib-2), (Ic-1), or (Ic-2), wherein HP² is a methoxypolyethylene glycoloxy-CH₂CH₂—C(O)— (mPEG-CH₂CH₂—C(O)—).

Embodiment [28]: The conjugate of embodiment [24] or [27], wherein the conjugate is of Formula (I), (Ib), (Ic), (Ib-1), (Ib-2), (Ic-1), or (Ic-2), wherein each W² and W⁰² is independently absent or of the formula:

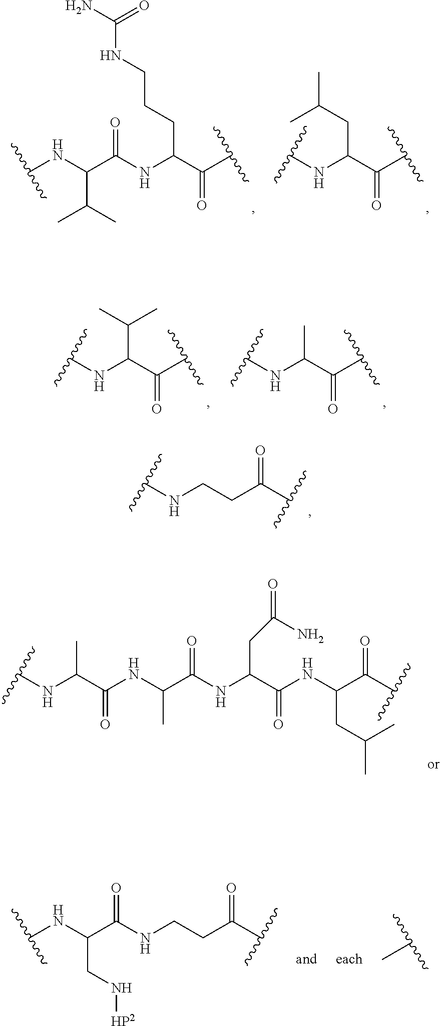

is the point of attachment to the rest of the formula.

Embodiment [28-1]: The conjugate of embodiment [24] or [27], wherein the conjugate is of Formula (I), (Ib), (Ic), (Ib-1), (Ib-2), (Ic-1), or (Ic-2), wherein each W² and W⁰² is independently of the formula:

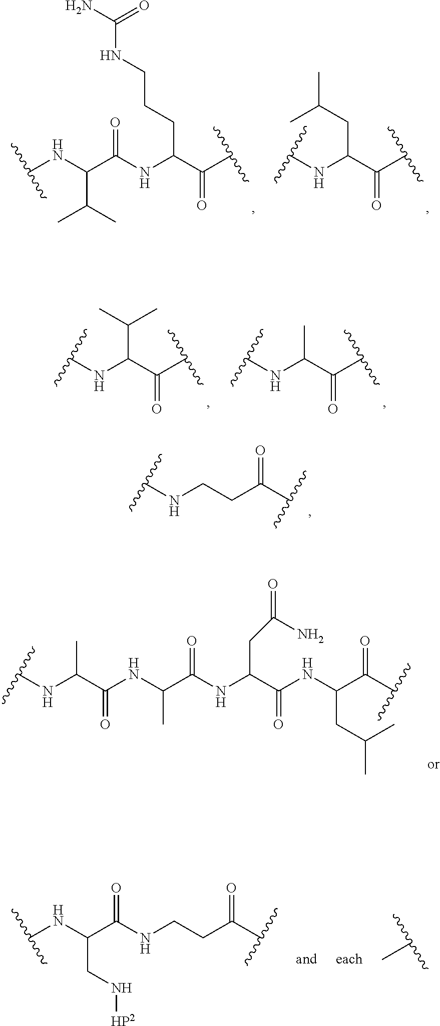

is the point of attachment to the rest of the formula.

Embodiment [28-2]: The conjugate of embodiment [28-1], wherein the conjugate is of Formula (I), (Ib), (Ic), (Ib-1), (Ib-2), (Ic-1), or (Ic-2), wherein W² is of the formula:

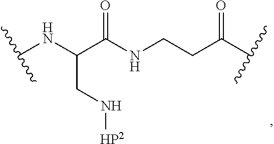

and W⁰² is of the formula:

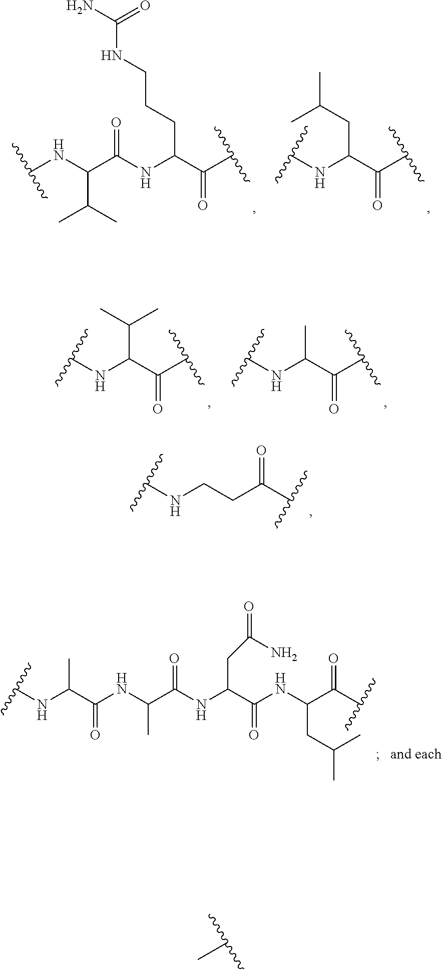

is the point of attachment to the rest of the formula.

Embodiment [29]: The conjugate of any one of embodiments [2] to [28], wherein the conjugate is of Formula (I), (Ib), (Ic), (Ib-1), (Ib-2), (Ic-1), or (Ic-2), wherein SG¹ is independently —C(O)—C₁₋₆ alkylene-C(O)—, and SG⁰¹ is independently absent or —C₁₋₆ alkylene-C(O)— wherein the —C(O)— is bound to W² and W⁰².

Embodiment [30]: The conjugate of any one of embodiments [2] to [29], wherein the conjugate is of Formula (I), (Ib), (Ic), (Ib-1), (Ib-2), (Ic-1), or (Ic-2), wherein each HP¹ and HP⁰¹ is independently absent or

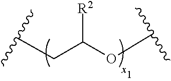

wherein R² is hydrogen or methyl and x1 is an integer between 1 and 50, inclusive; and each [symbol] is the point of attachment to the rest of the formula.

Embodiment [30-1]: The conjugate of embodiment [2] to [29], wherein the conjugate is of Formula (I), (Ib), (Ic), (Ib-1), (Ib-2), (Ic-1), or (Ic-2), wherein each HP¹ is absent and HP⁰¹ is independently absent or

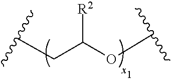

wherein R² is hydrogen or methyl and x1 is an integer between 1 and 50, inclusive; and each [symbol] is the point of attachment to the rest of the formula.

Embodiment [30-2]: The conjugate of embodiment [2] to [29], wherein the conjugate is of Formula (I), (Ib), (Ic), (Ib-1), (Ib-2), (Ic-1), or (Ic-2), wherein each HP¹ and HP⁰¹ is independently absent,

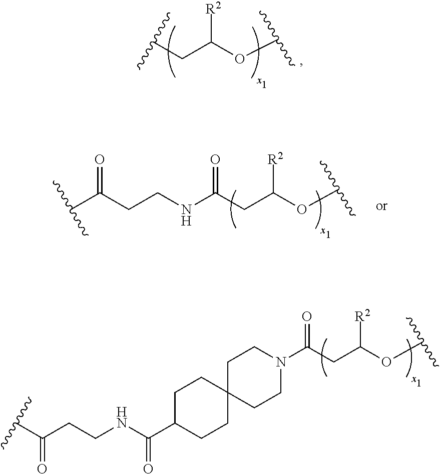

wherein R² is hydrogen or methyl and x1 is an integer between 1 and 50, inclusive; and each [symbol] is the point of attachment to the rest of the formula.

Embodiment [30-3]: The conjugate of embodiment [2] to [29], wherein the conjugate is of Formula (I), (Ib), (Ic), (Ib-1), (Ib-2), (Ic-1), or (Ic-2), wherein each HP¹ is independently absent,

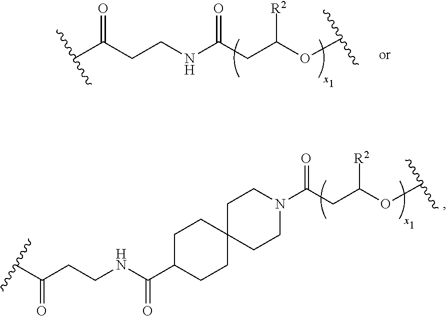

and HP⁰¹ is independently absent or

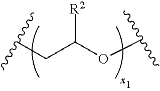

wherein R² is hydrogen or methyl and x1 is an integer between 1 and 50, inclusive; and each [symbol] is the point of attachment to the rest of the formula.

Embodiment [31]: The conjugate of any one of embodiments [2] to [30], wherein the conjugate is of Formula (I), (Ib), (Ic), (Ib-1), (Ib-2), (Ic-1), or (Ic-2)], wherein each RL and RL⁰¹ is independently selected from

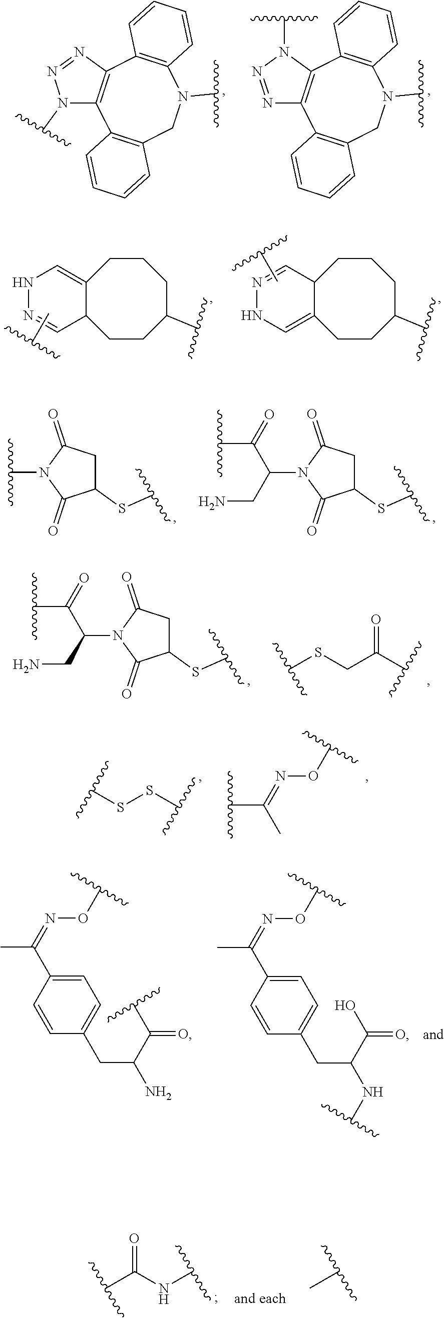

is a point of attachment to the rest of the formula.

Embodiment [32]: The conjugate of embodiment [31], wherein the conjugate is of Formula (I), (Ib), (Ic), (Ib-1), (Ib-2), (Ic-1), or (Ic-2), wherein RL¹ is independently selected from

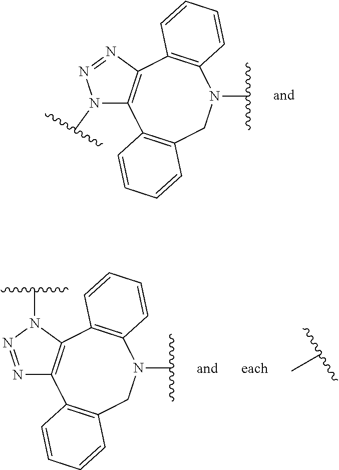

is a point of attachment to the rest of the formula.

Embodiment [33]: The conjugate of embodiment [32], wherein the conjugate is of Formula (I), (Ib), (Ic), (Ib-1), (Ib-2), (Ic-1), or (Ic-2), wherein RL⁰¹ is

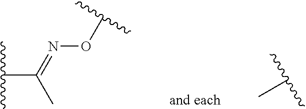

is a point of attachment to the rest of the formula.

Embodiment [34]: The conjugate of any one of embodiments [2] to [33], wherein the conjugate is of Formula (I), (Ib), (Ic), (Ib-1), (Ib-2), (Ic-1), or (Ic-2), wherein CY is independently selected from

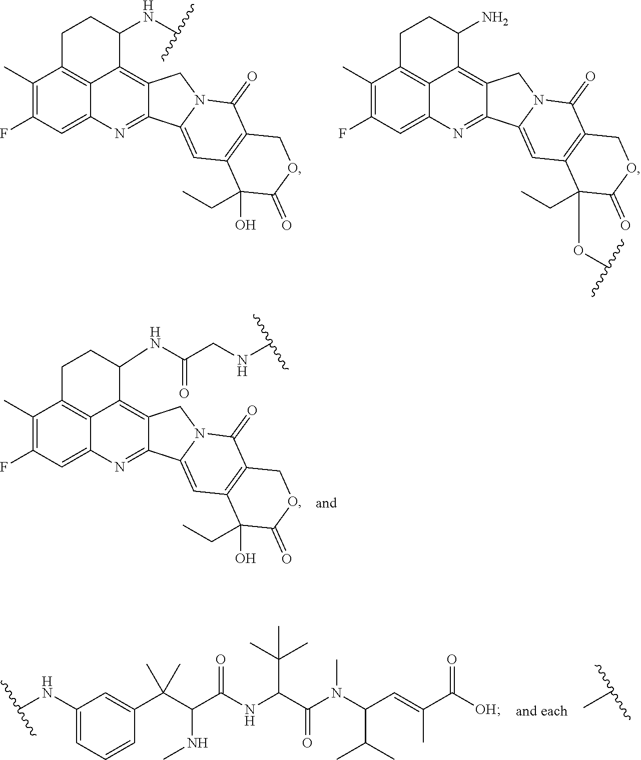

is a point of attachment to the rest of the formula.

Embodiment [35]: The conjugate of embodiment [34], wherein the conjugate is of Formula (I), (Ib), (Ic), (Ib-1), (Ib-2), (Ic-1), or (Ic-2), wherein CY is independently selected from

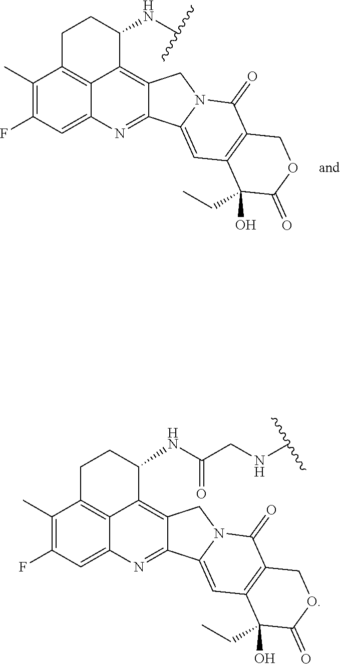 [SMILES: *N[C@H]1CCc2c(C)c(F)cc3nc4c(c1c23)Cn1c-4cc2c(c1=O)COC(=O)[C@]2(O)CC ; *NCC(=O)N[C@H]1CCc2c(C)c(F)cc3nc4c(c1c23)Cn1c-4cc2c(c1=O)COC(=O)[C@]2(O)CC]

Embodiment [36]: The conjugate of any one of embodiments [1]-[35], wherein the conjugate is of Formula (I) or (Ia), wherein -[L^{b}-STING]ₙ is independently

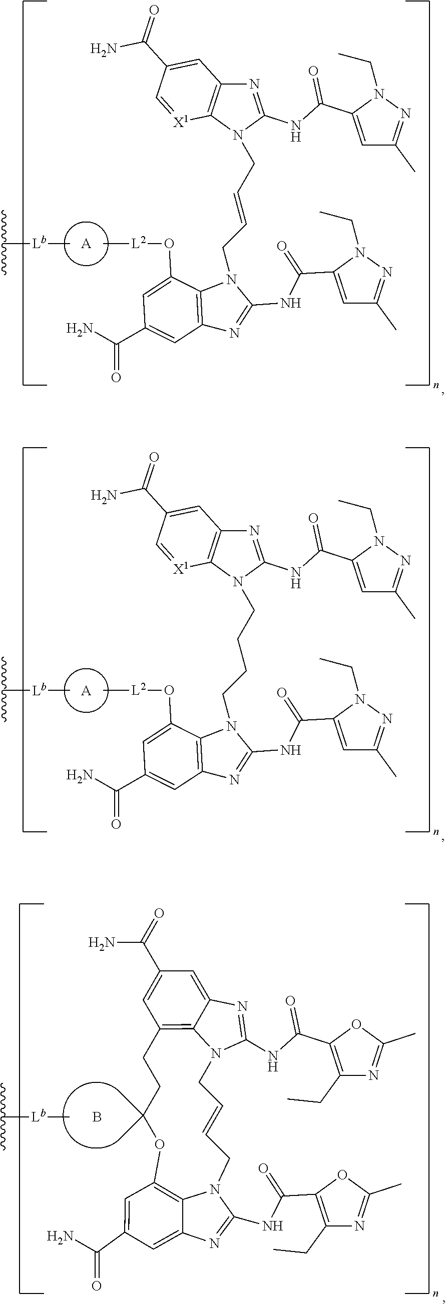

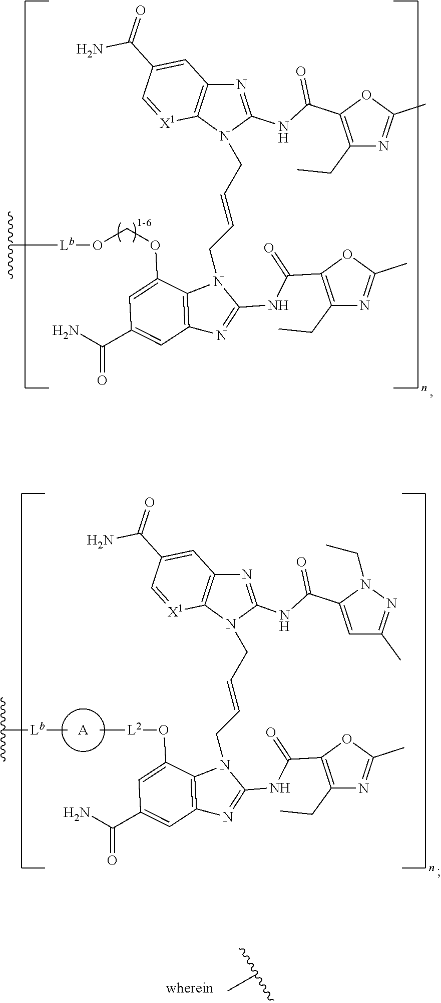

is the point of attachment to the rest of the formula.

Embodiment [37]: The conjugate of any one of embodiments [1]-[36], wherein the conjugate of Formula (I) or (Ia) wherein -[L^{b}-STING]ₙ is a structure selected from:

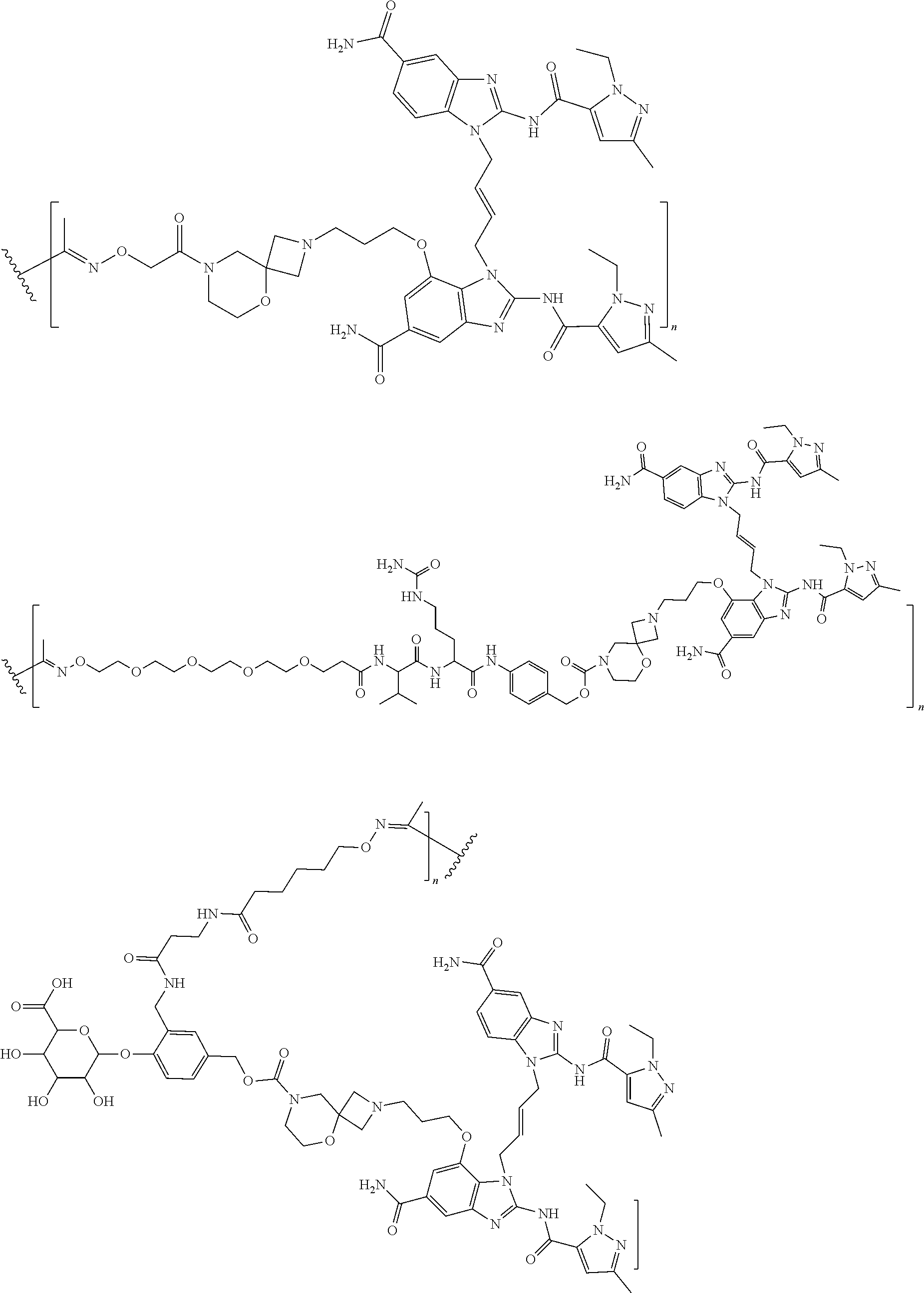

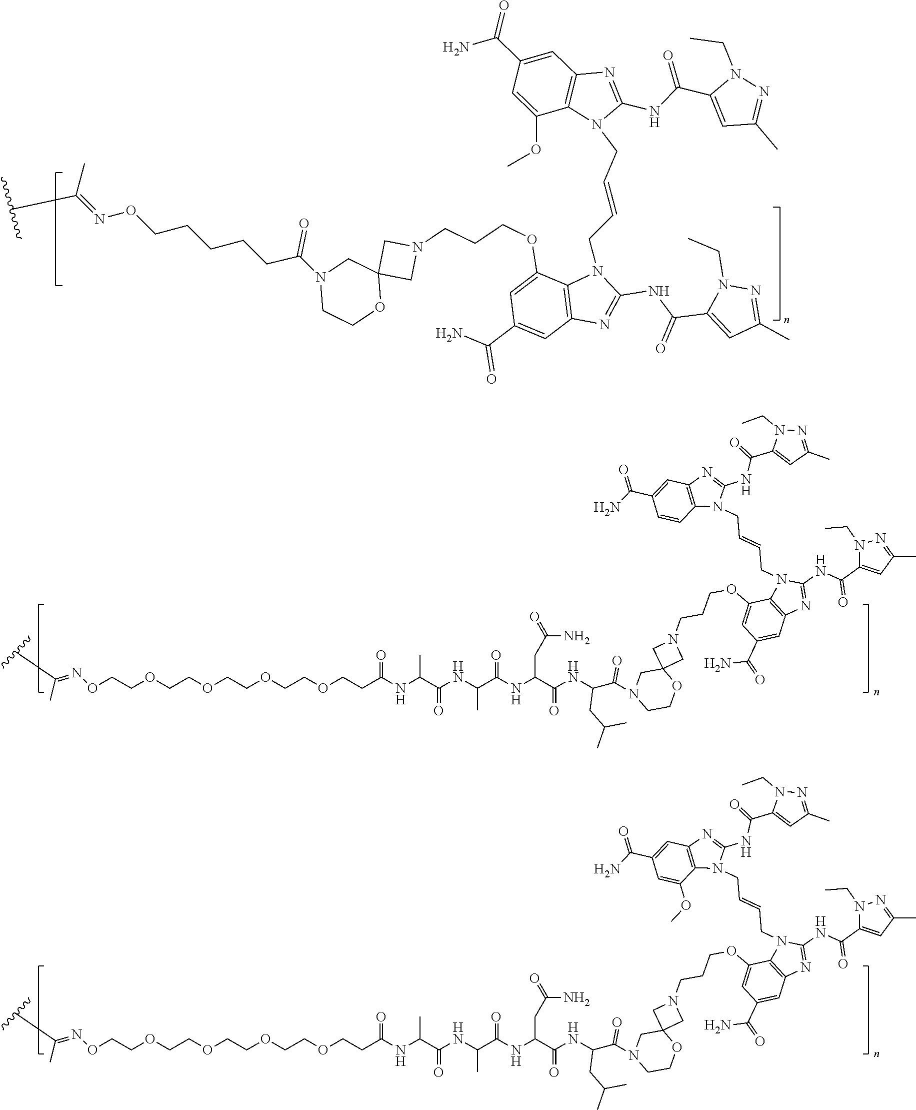

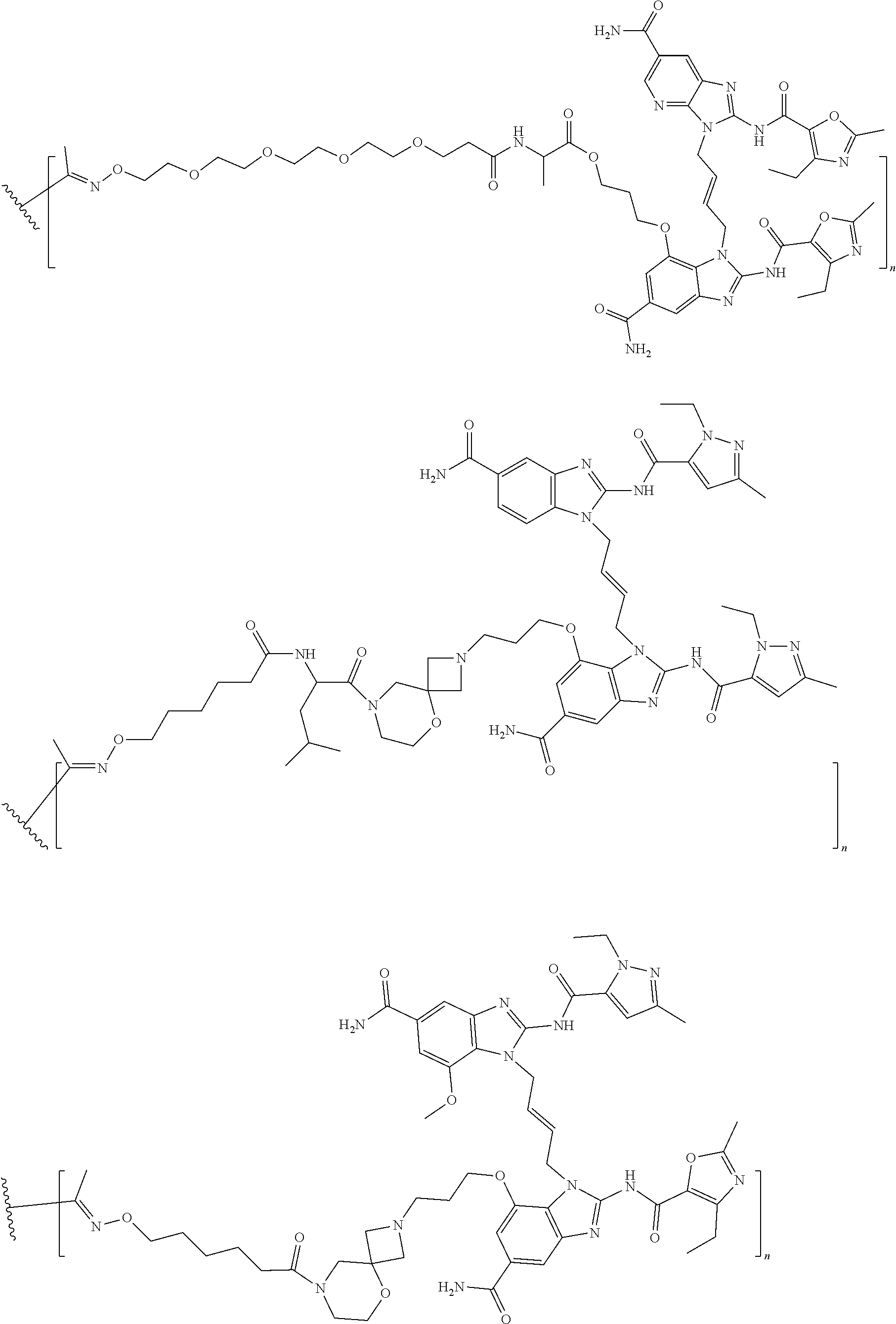

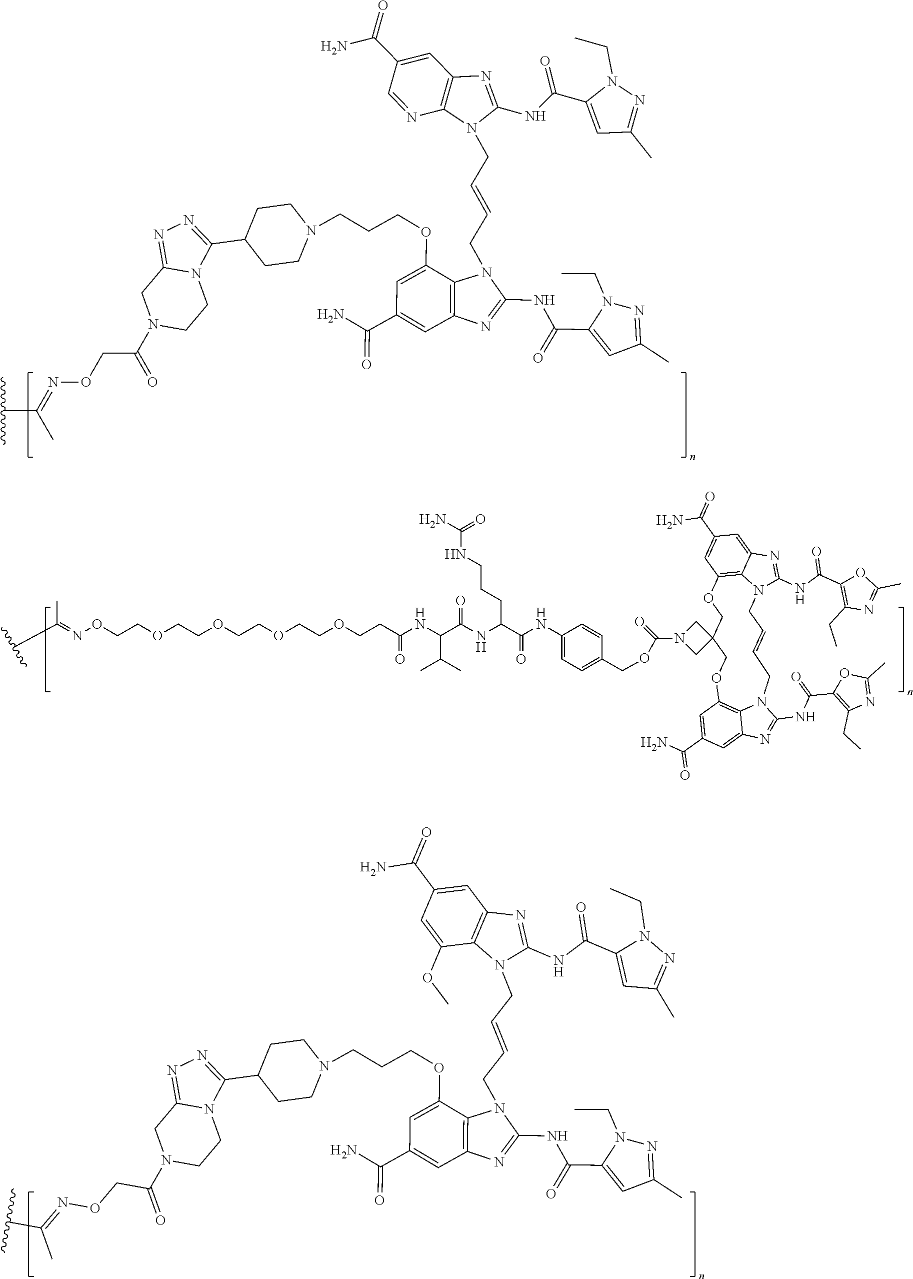

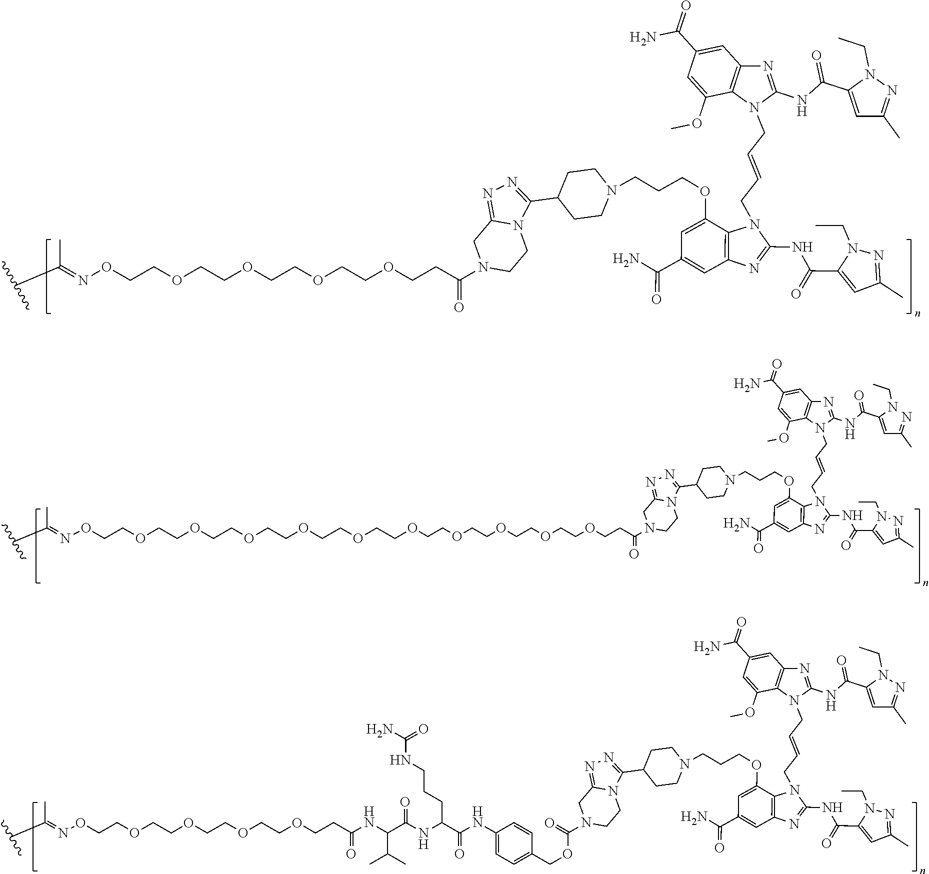

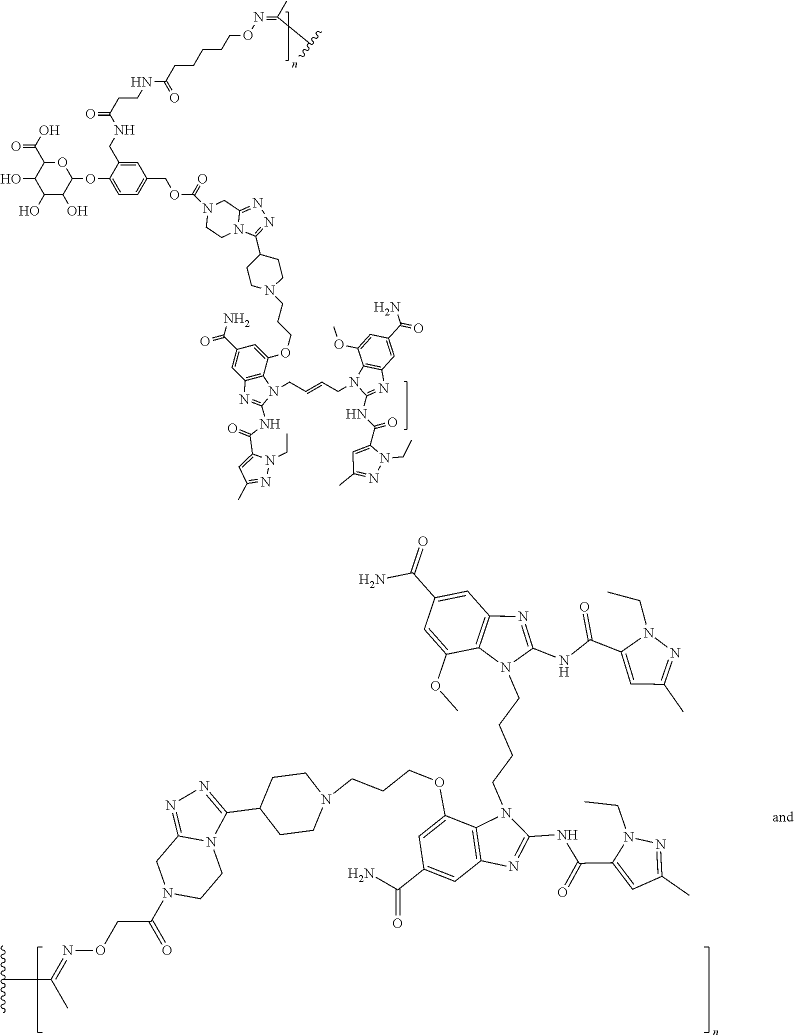

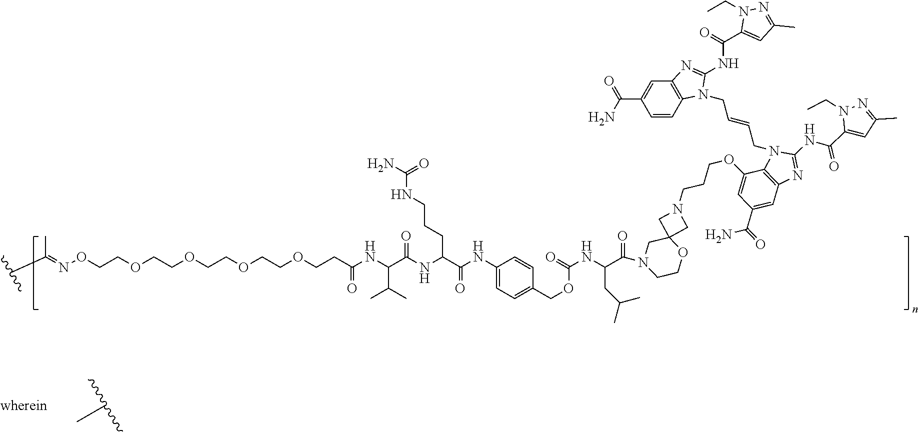

is the point of attachment to the rest of the formula.

Embodiment [38]: The conjugate of any one of embodiments [1] to [37], wherein the conjugate is of Formula (I), (Ib), (Ic), (Ib-1), (Ib-2), (Ic-1), or (Ic-2), wherein

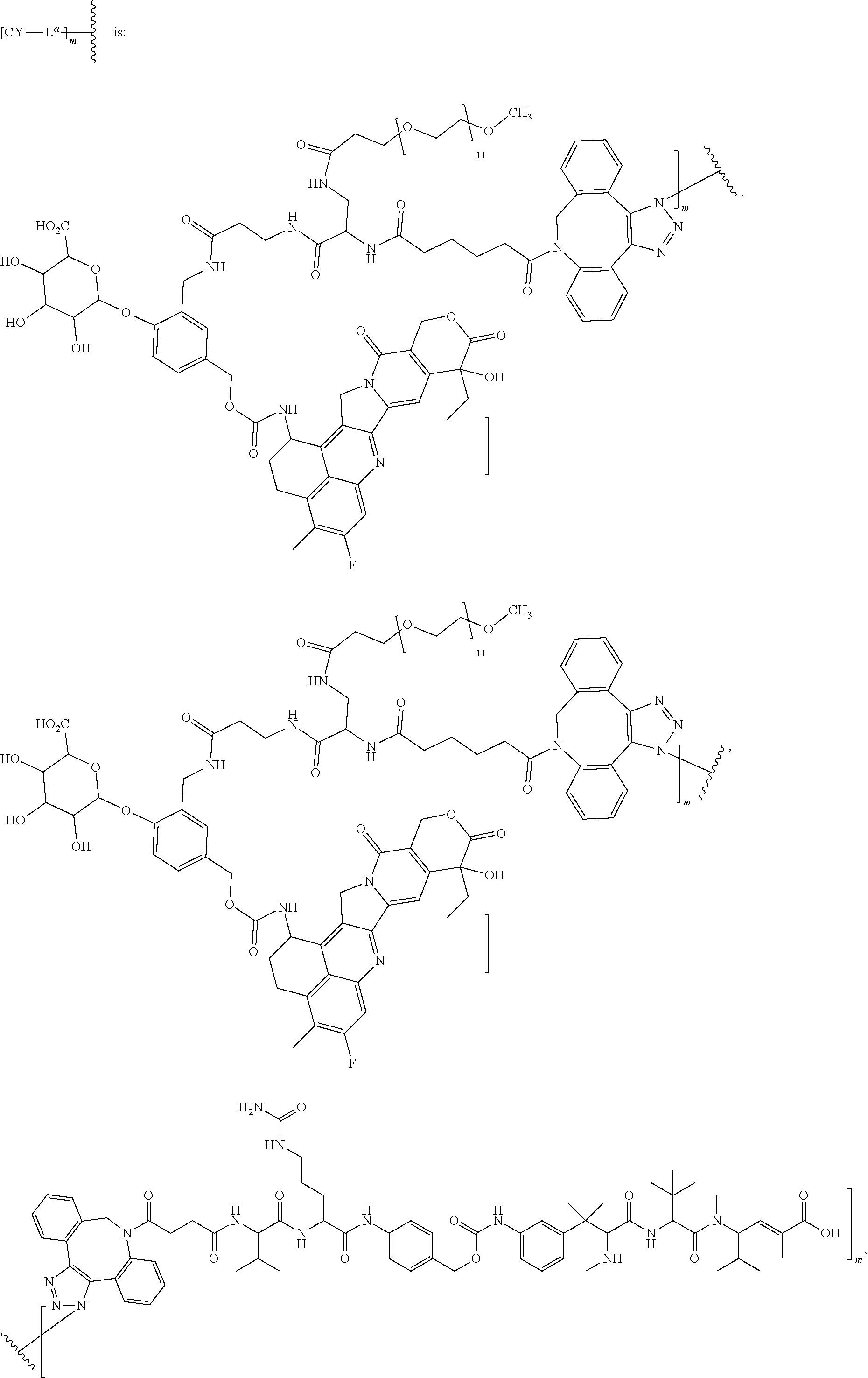

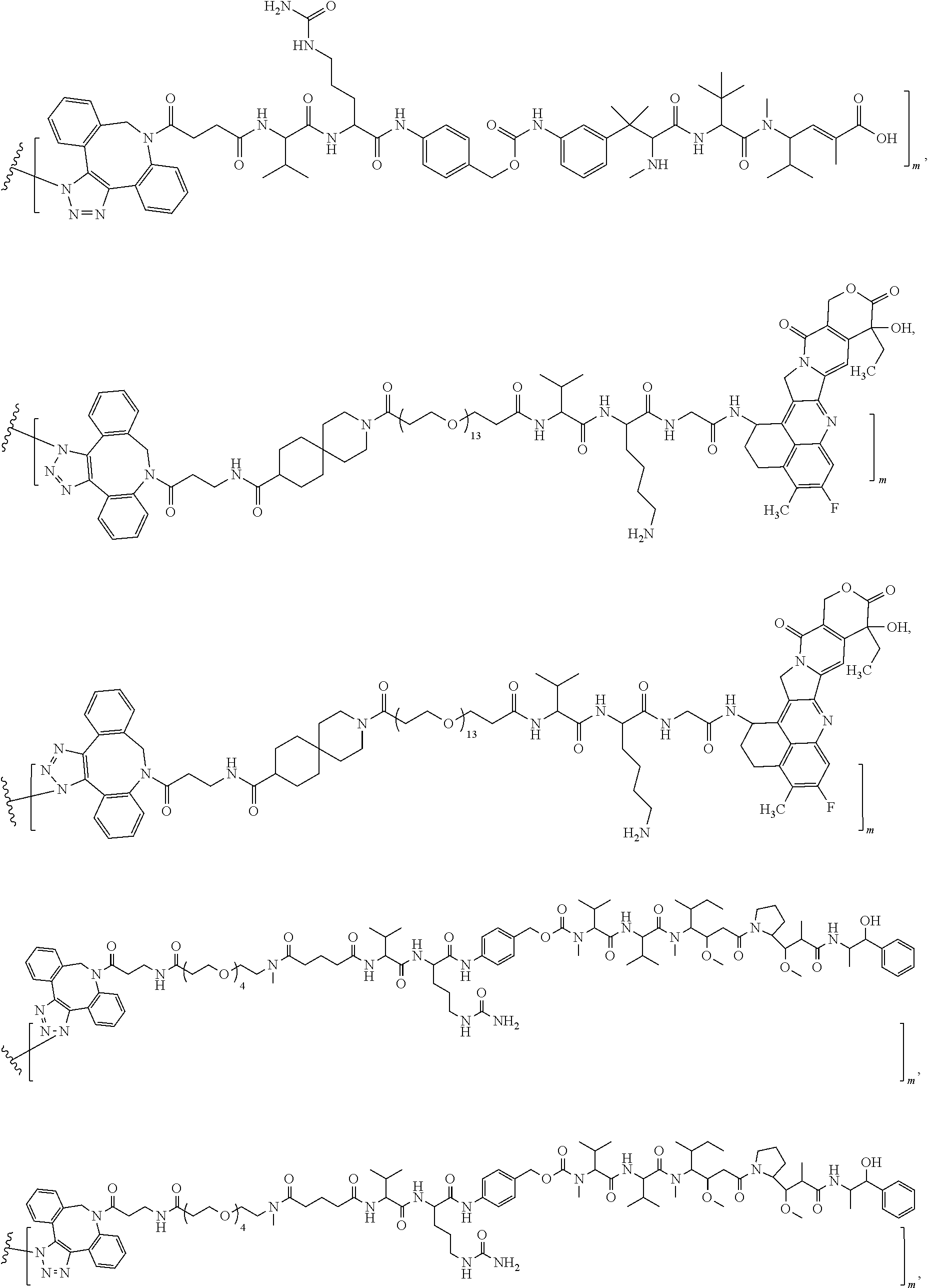

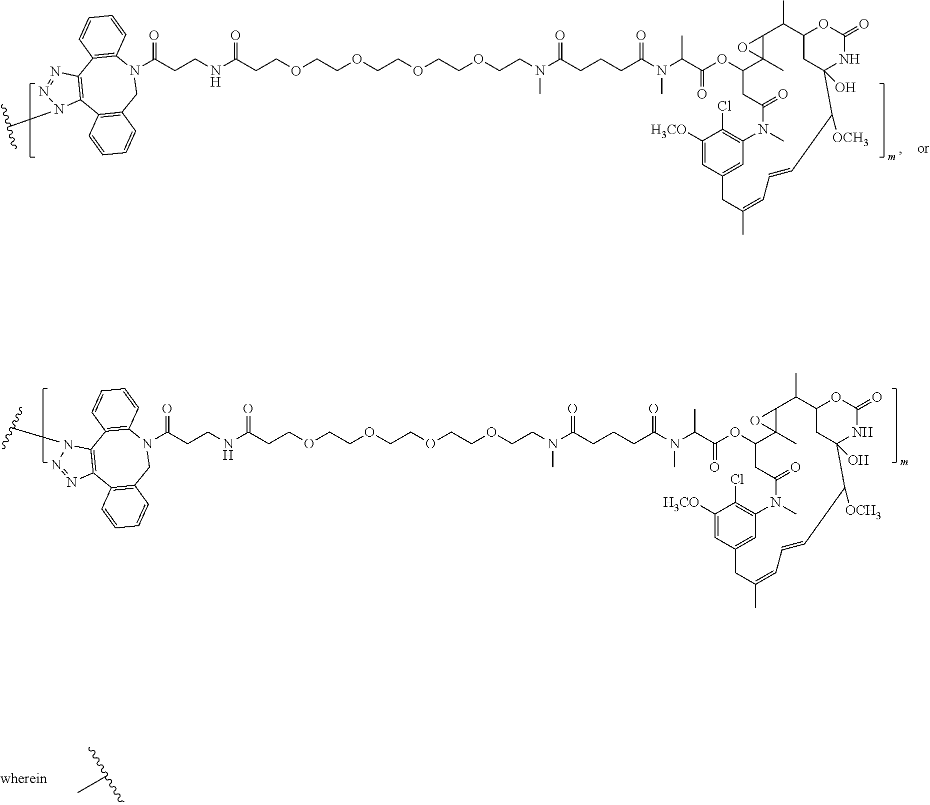

is the point of attachment to the rest of the formula.

Embodiment [39]: The conjugate of any one of embodiments [1] to [38], wherein the conjugate is of Formula (I), (Ib), (Ic), (Ib-1), (Ib-2), (Ic-1), or (Ic-2), wherein
[CY-L^{a}-]ₘ- is

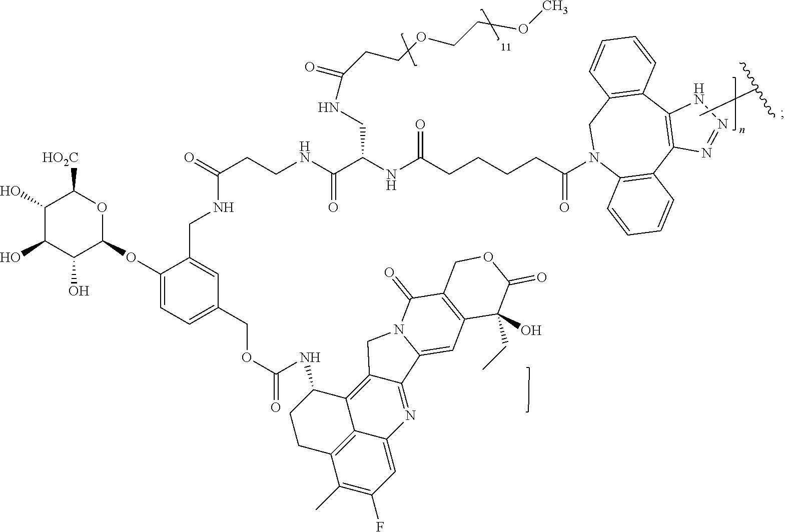

and
-[L^{b}-STING]ₙ is

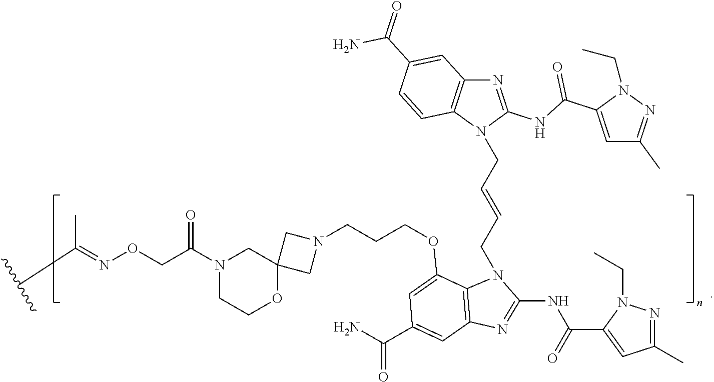

Embodiment [40]: The conjugate of any one of embodiments [1] to [39], wherein the conjugate is of Formula (I), (Ib), (Ic), (Ib-1), (Ib-2), (Ic-1), or (Ic-2), wherein
[CY-L^{a}-]ₘ- is

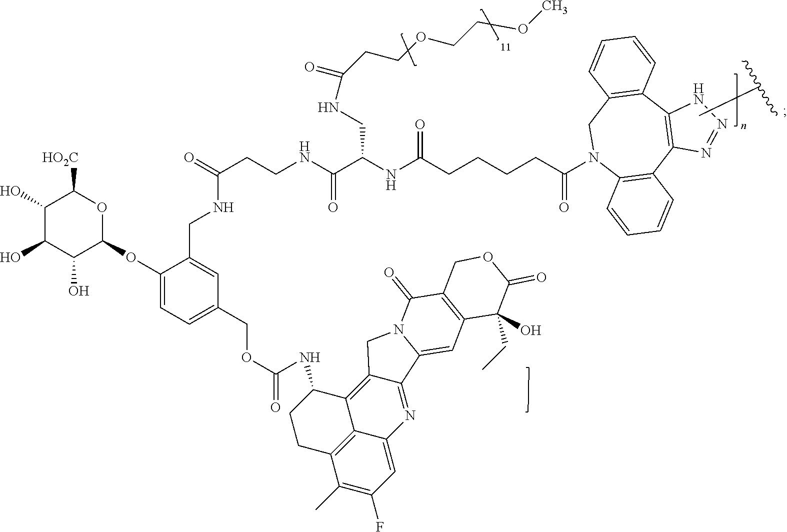

and
-[L^{b}-STING]ₙ is a structure selected from

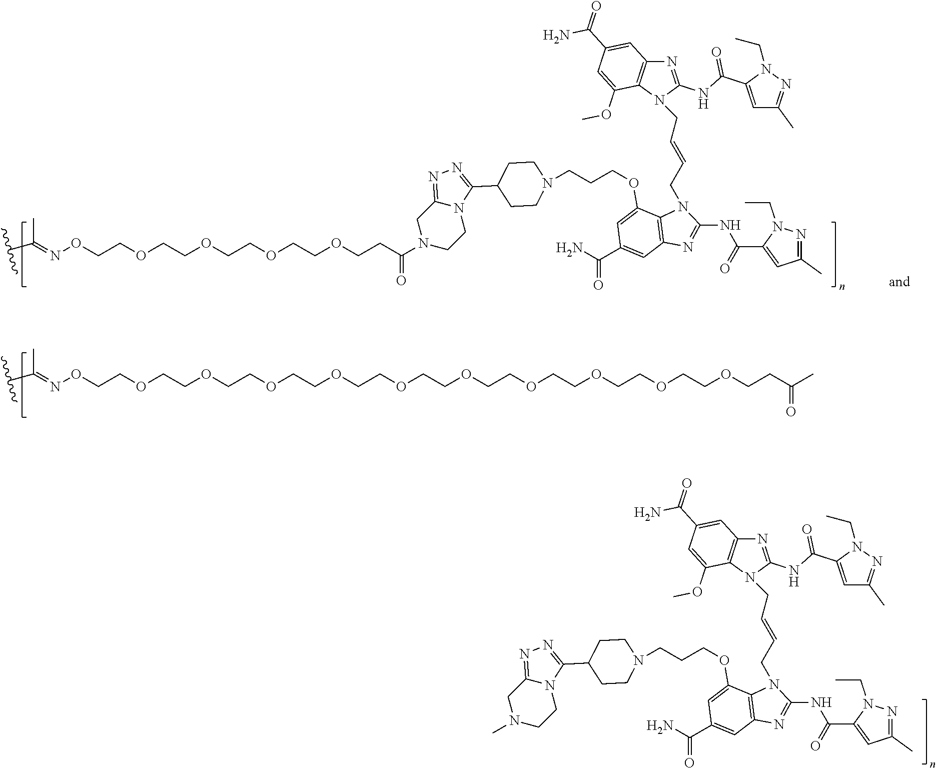

Embodiment [41]: The conjugate of any one of embodiments [1] to [40], wherein of m and n is an integer independently between 1 and 8, inclusive.

Embodiment [42]: The conjugate of any one of embodiments [1] to [41], wherein n is between 1 and 6, inclusive.

Embodiment [43]: The conjugate of any one of embodiments [1] to [42], wherein m is between 1 and 6, inclusive.

Embodiment [44]: The conjugate of any one of embodiments [1] to [43], wherein of m is an integer independently between 1 and 6 and n is an integer independently between 1 and 6, inclusive.

Embodiment [45]: The conjugate of any one of embodiments [1] to [44], wherein the molar ratio of CY to STING is about 2:1.

Embodiment [46]: The conjugate of any one of embodiments [1] to [44], wherein the molar ratio of m to n is about 2:1.

Embodiment [47]: The conjugate is of Formula (Ia):

[CY-W¹—W²-SG¹-HP¹-RL¹-]ₘ-Ab-[-RL⁰¹-HP⁰¹-SG⁰¹-W⁰²-W⁰¹-STING]ₙ
wherein CY is independently a residue of a cytotoxic agent;
W¹ and W¹ are independently

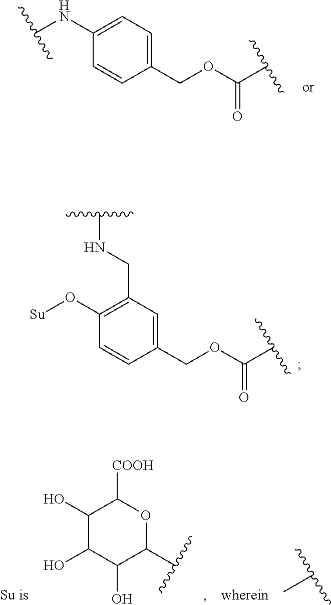

represents attachment to the remainder of the compound;
W² and W⁰² are independently absent or —(C(O)CH₂CHR¹⁰NR¹)ₚ—(C(O)CHR¹⁰NR¹¹)ₒ—;
wherein
R¹⁰ is an amino acid sidechain residue independently selected from valine, citrulline, alanine, glycine, leucine, asparagine, lysine and 2,3-diaminopropionate and wherein the amino acid sidechain is optionally substituted with HP²;
HP² is independently polyethylene glycol (PEG) or methoxypolyethylene glycol (mPEG) thereof;
R¹¹ is hydrogen;
is independently an integer between 1 and 4, inclusive;
p is 1;
SG¹ is independently —C(O)—C₁₋₆ alkylene-C(O)—, and SG⁰¹ is independently absent or —C₁₋₆ alkylene-C(O)— wherein the —C(O)— is bound to W² and W⁰²;
HP¹ and HP⁰¹ is independently absent or

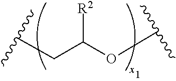

wherein R² is hydrogen or methyl and x1 is an integer between 1 and 50, inclusive; and each [symbol] is the point of attachment to the rest of the formula; [symbol]
RL¹ and RL⁰¹ are independently, at each occurrence, a reactive linker;

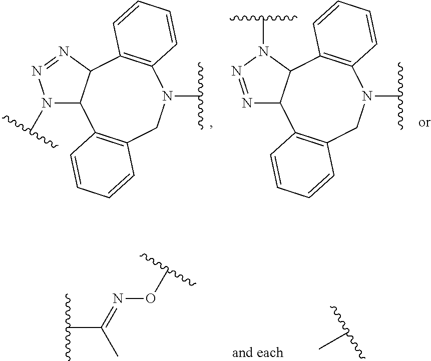

is a point of attachment to the rest of the formula;
STING is independently a residue of a STING agonist;
Wherein the STING agonist is a residue of a compound of Formula (S-I) or (S-II):

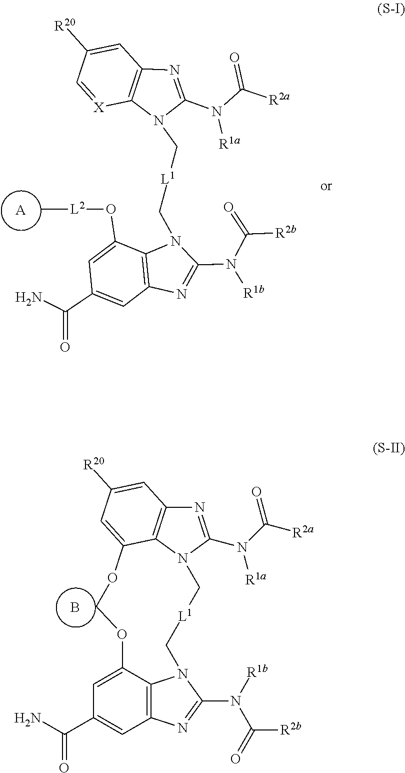

or a tautomer or stereoisomer thereof;
wherein:
X is CR³;
R²⁰ is —CON(R^{3a})(R^{3b});
R^{1a}, R^{1b}, R^{3a} and R^{3b} are independently hydrogen;
R^{2a} and R^{2b} are independently selected from:

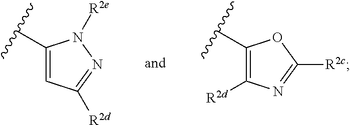

wherein R^{2c}, R^{2d} and R^{2e} are independently C₁₋₆ alkyl;
R³ is hydrogen, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, or —CN;
L¹ is —C₂₋₁₀ alkenylene-;
L² is —C₁₋₆ alkylene-;
Ring A is a)

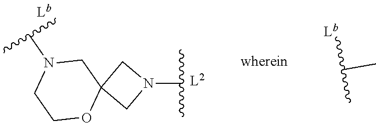

is the point of attachment to L^{b} and [symbol] is the point of attachment to L²; or b)

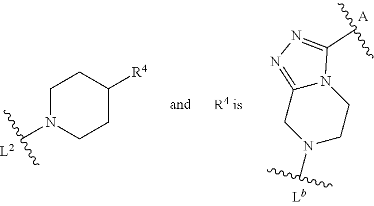

Ring B is azetidine;
R³⁰ is independently selected from hydrogen and C₁₋₁₀ alkyl;
wherein the residue of Formula (S-I) is attached to L^{b} via an amino group of Ring A, the oxygen of —C₁₋₆ alkylene-O— of L², or an amino group of R⁴, and the residue of Formula (S-II) is attached to L^{b} via an amino group of Ring B;
subscript n is an integer selected from 1 to 10; and
subscript m is an integer selected from 1 to 10.

Embodiment [48]: The antibody conjugate according to Embodiment [47], wherein CY is a topoisomerase I inhibitor selected from the group consisting of camptothecin, irinotecan, SN-38, topotecan, and exatecan.

Embodiment [49]: The antibody conjugate according to Embodiment [47] or [48], wherein CY is exatecan.

Embodiment [50]: The antibody conjugate according to any one of Embodiments [47]-[49], wherein W² and W⁰² is independently absent or of the formula:

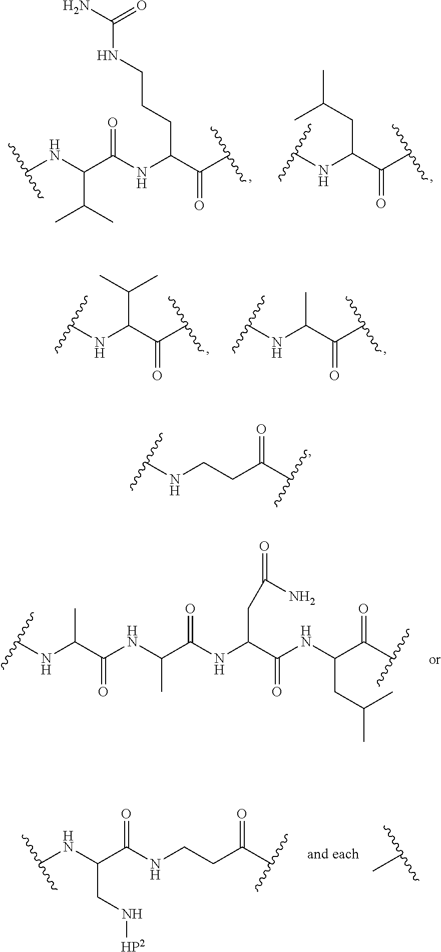

is the point of attachment to the rest of the formula.

### 1. Linkers

The first linker (L^{a}) and the second linker (L^{b}) can be any linkers capable of forming at least one bond to the anti-TROP2 antibody and at least one bond to a payload wherein the first linker forms at least one bond with the either the STING agonist and the second linker forms a bond with the cytotoxic agent. The first linker and the second linker can have the same structure, but in a preferable embodiment, the first linker and the second linker are different structures. In some embodiments, including any of the foregoing, the first and second linker independently comprise a protease cleavable linker, an enzyme cleavable linker, a pH-sensitive linker, or a non-cleavable linker.

In certain embodiments of L^{a} and L^{b}, W¹ and W⁰¹ are independently absent,

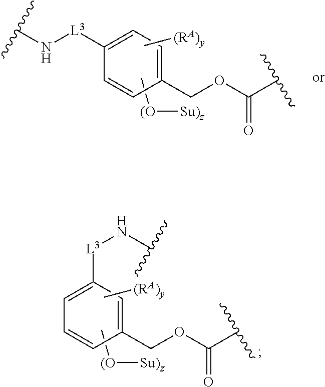

L³ is bond or —CH₂—; y is 0 or 1; and, z is 0 or 1.

In certain embodiments, Su is

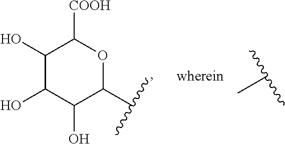

wherein represents attachment to the remainder of the compound.

In certain embodiments, Su is

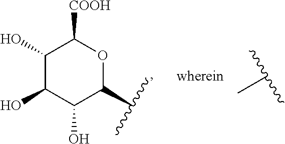

wherein represents attachment to the remainder of the compound.

In certain embodiments, each W² and W⁰² is independently absent or a group selected from —(C(O)CHR¹⁰NR¹¹)ₒ—, —(C(O)CH₂CHR¹⁰NR¹¹)ₚ—, —(C(O)CHR¹⁰CH₂NR¹¹)ₚ—, —(C(O)CH₂CHR¹⁰NR¹¹)ₚ—(C(O)CHR¹⁰NR¹¹)ₒ—, and —(C(O)CHR¹⁰CH₂NR¹¹)ₚ—(C(O)CHR¹⁰NR¹¹)ₒ—;
wherein R¹⁰ is independently an amino acid sidechain residue optionally substituted with HP²;
R¹¹ is independently hydrogen or C₁₋₆ alkyl;
and p is independently an integer between 1 and 10, inclusive; and
the —C(O)— of W² and W⁰² is bound to W¹.

In certain embodiments, each W² and W⁰² is independently absent or —(C(O)CH₂CHR¹⁰NR¹¹)ₚ—(C(O)CHR¹⁰NR¹¹)ₒ— wherein
R¹⁰ is an amino acid sidechain residue independently selected from valine, citrulline, alanine, glycine, leucine, asparagine, lysine and 2,3-diaminopropionate and wherein the amino acid sidechain is optionally substituted with HP²;
HP² is independently polyethylene glycoloxy-CH₂CH₂C(O)— (PEG-CH₂CH₂C(O)—); methoxypolyethylene glycoloxy-CH₂CH₂C(O)— (mPEG-CH₂CH₂C(O)—); poly(propylene glycoloxy-CH₂CH₂C(O)—) (PPG-CH₂CH₂C(O)—); poly(oxyethylated polyol)oxy-CH₂CH₂C(O)—; poly(olefinic alcohol)oxy-CH₂CH₂C(O)—; poly(saccharides)-CH₂CH₂C(O)—; poly(vinyl alcohol)oxy-CH₂CH₂C(O)—; or a combination thereof,
R¹¹ is hydrogen;
is independently an integer between 1 and 4, inclusive; and
p is 1.

In certain embodiments, each W² and W⁰² is independently absent or —(C(O)CH₂CHR¹⁰NR¹¹)ₚ—(C(O)CHR¹⁰NR¹¹)ₒ— wherein
R¹⁰ is an amino acid sidechain residue independently selected from valine, citrulline, alanine, glycine, and 2,3-diaminopropionate and wherein the amino acid sidechain is optionally substituted with HP²;
HP² is independently polyethylene glycoloxy-CH₂CH₂C(O)— (PEG-CH₂CH₂C(O)—); methoxypolyethylene glycoloxy-CH₂CH₂C(O)— (mPEG-CH₂CH₂C(O)—); poly(propylene glycoloxy-CH₂CH₂C(O)—) (PPG-CH₂CH₂C(O)—); poly(oxyethylated polyol)oxy-CH₂CH₂C(O)—; poly(olefinic alcohol)oxy-CH₂CH₂C(O)—; poly(saccharides)-CH₂CH₂C(O)—; poly(vinyl alcohol)oxy-CH₂CH₂C(O)—; or a combination thereof,
R¹¹ is hydrogen;
is independently an integer between 1 and 4, inclusive; and
p is 1.

In certain embodiments, HP² is a methoxypolyethylene glycol (mPEG).

In certain embodiments, each W² and W⁰² is independently absent or of the formula:

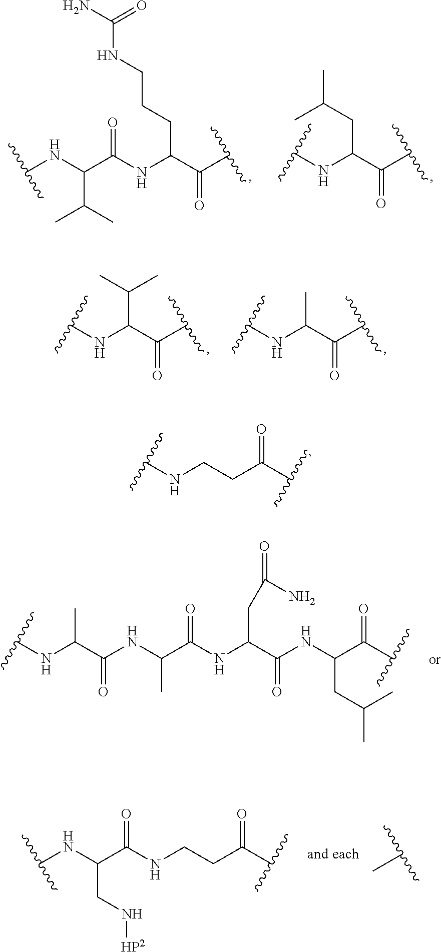

is the point of attachment to the rest of the formula.

In certain embodiments, each W² and W⁰² is independently of the formula:

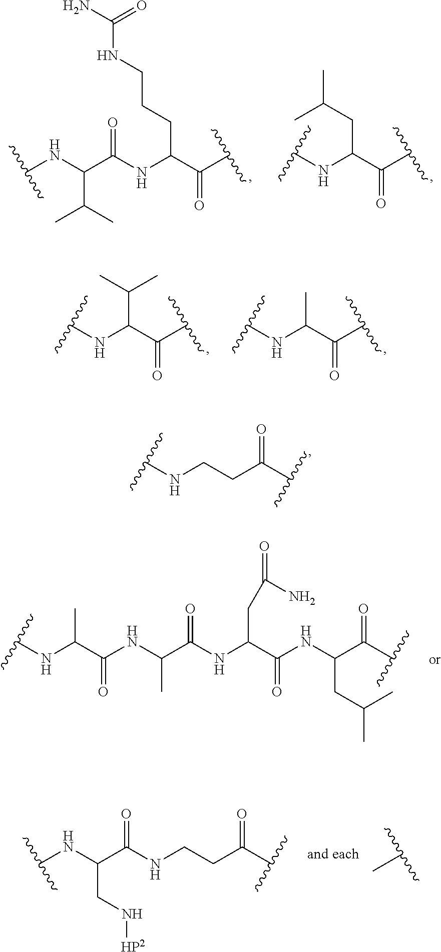

is the point of attachment to the rest of the formula.

In certain embodiments, SG¹ is independently —C(O)—C₁₋₆ alkylene-C(O)—, and SG⁰¹ is independently absent or —C₁₋₆ alkylene-C(O)— wherein the —C(O)— is bound to W² and W⁰².

In certain embodiments, each HP¹ and HP¹ is independently absent or

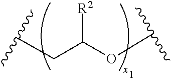

wherein R² is hydrogen or methyl and x1 is an integer between 1 and 50, inclusive; and each [symbol] is the point of attachment to the rest of the formula.

In certain embodiments, each RL and RL⁰¹ is independently selected from

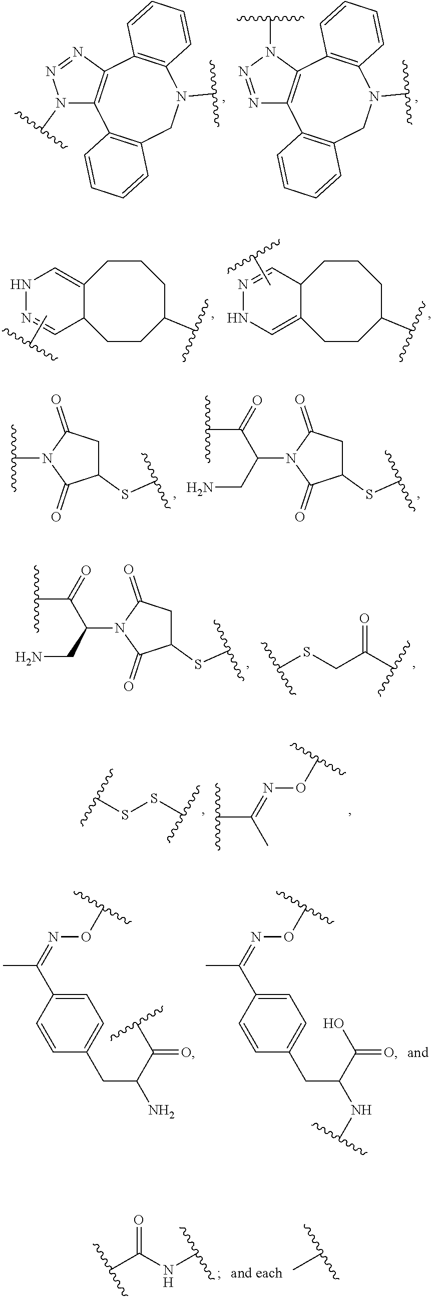

is a point of attachment to the rest of the formula.

In certain embodiments, RL¹ is independently selected from

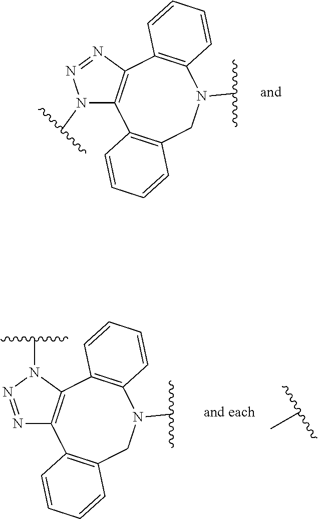

is a point of attachment to the rest of the formula.

In certain embodiments, RL⁰¹ is

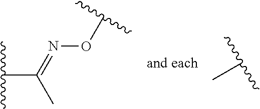

is a point of attachment to the rest of the formula.

In certain embodiments, SG¹ and SG⁰¹ are divalent spacer groups that facilitate incorporation of eliminator groups, release trigger groups, hydrophobic groups, attaching groups, and/or the conjugating group into a compound. Spacer groups that facilitate the spacing of the conjugating group from the other groups of the compounds can lead to more efficient conjugation of the compounds described herein to a second compound as well as more efficient cleavage of the active catabolite. Spacer groups can also stabilize the conjugating group and lead to improved overall antibody-drug conjugate properties. Useful spacer groups are known to, and are apparent to, those of skill in the art. Examples of useful spacer groups are provided herein. In certain embodiments, a spacer group can comprise a divalent ketone, divalent ester, divalent ether, divalent amide, divalent amine, alkylene, arylene, sulfide, disulfide, carbonylene, or a combination thereof. In certain embodiments a spacer group can comprise —C(O)—, —O—, —C(O)NH—, —C(O)NH-alkyl-, —OC(O)NH—, —SC(O)NH—, —NH—, —NH-alkyl-, —C(O)N(CH₃)—, —C(O)N(CH₃)-alkyl-, —N(CH₃)—, —N(CH₃)-alkyl-, —N(CH₃)CH₂CH₂N(CH₃)—, —C(O)CH₂CH₂CH₂C(O)—, —S—, —S—S—, —OCH₂CH₂O—, or the reverse (e.g. —NHC(O)—) thereof, or a combination thereof.

In certain embodiments of Formula (Ia), SG¹ and SG⁰¹ are independently absent or selected from —C₁₋₆ alkylene-, —C(O)—, —C₁₋₆ alkylene-C(O)—, —C(O)(C₁₋₆ alkylene)-C(O)—NR¹C₁₋₆alkylene- wherein the —C(O)— is bound to W² or W⁰², respectively, —C(O)—C₁₋₆ alkylene-C(O)—, —C(O)(C₁₋₆ alkylene)NR¹C(O)—, —C(O)(C₁₋₆ alkylene)OC(O)—, and —C(O)(C₁₋₆ alkylene)SC(O)—; wherein R¹ is hydrogen or optionally substituted C₁₋₆ alkyl; and the C₁₋₆ alkylene of SG¹ or SG⁰¹, alone or part of another group, is optionally substituted with one, two, or three substituents selected from halogen, haloC₁₋₆alkyl, hydroxyl, amino, C₁₋₆alkylamino, and C₁₋₆alkoxy.

In certain embodiments of Formula (Ia), including any of the foregoing, SG¹ and SG⁰¹ are independently absent, —C(O)(C₁₋₆ alkylene)OC(O)—, —C₁₋₆ alkylene-C(O)—, or —C(O)(C₁₋₆ alkylene)-C(O)—NR¹C₁₋₆alkylene- wherein the —C(O)— is bound to W² or W⁰², respectively. In certain embodiments of Formula (Ia), including any of the foregoing, SG¹ is —C(O)(C₁₋₆ alkylene)OC(O)—. In certain embodiments of Formula (Ia), including any of the foregoing, SG¹ is —C(O)(C₂₋₄ alkylene)OC(O)—. In certain embodiments of Formula (Ia), including any of the foregoing, SG¹ is —C(O)(C₁₋₆ alkylene)-C(O)—NR¹C₁₋₆alkylene- wherein the —C(O)— is bound to W². In certain embodiments of Formula (Ia), including any of the foregoing, SG¹ is —C(O)(C₃ alkylene)-C(O)—N(CH₃)C₂alkylene- wherein the —C(O)— is bound to W². In certain embodiments of Formula (Ia), including any of the foregoing, SG¹ is —C₁₋₆ alkylene-C(O)— wherein the —C(O)— is bound to W⁰².

In certain embodiments of Formula (Ib), (Ic), (Ib-1), (Ib-2), (Ic-1), or (Ic-2), SG¹ and SG⁰¹ are independently absent or selected from —C₁₋₆ alkylene-, —C(O)—, —C₁₋₆ alkylene-C(O)—, —C(O)(C₁₋₆ alkylene)-C(O)—NR¹C₁₋₆alkylene- wherein the —C₁₋₆ alkylene- is bound to Y, —C(O)—C₁₋₆ alkylene-C(O)—, —C(O)(C₁₋₆ alkylene)NR¹C(O)—, —C(O)(C₁₋₆ alkylene)OC(O)—, and —C(O)(C₁₋₆ alkylene)SC(O)—; wherein R¹ is hydrogen or optionally substituted C₁₋₆ alkyl; and the C₁₋₆ alkylene of SG¹ or SG⁰¹, alone or part of another group, is optionally substituted with one, two, or three substituents selected from halogen, haloC₁₋₆alkyl, hydroxyl, amino, C₁₋₆alkylamino, and C₁₋₆alkoxy.

In certain embodiments of Formula (Ib), (Ic), (Ib-1), (Ib-2), (Ic-1), or (Ic-2), SG¹ and SG⁰¹ are independently —C₁₋₆ alkylene-. In certain embodiments of Formula (Ib), (Ic), (Ib-1), (Ib-2), (Ic-1), or (Ic-2), SG¹ and SG⁰¹ are —CH₂—.

In certain embodiments, W¹ and W⁰¹ are eliminator groups. Eliminator groups facilitate separation of a biologically active portion of a compound or conjugate described herein from the remainder of the compound or conjugate in vivo and/or in vitro. Eliminator groups can also facilitate separation of a biologically active portion of a compound or conjugate described herein in conjunction with a release trigger group. For example, the eliminator group and the release trigger group can react in a Releasing Reaction to release a biologically active portion of a compound or conjugate described herein from the compound or conjugate in vivo and/or in vitro. Upon initiation of the Releasing Reaction by the release trigger, the eliminator group cleaves the biologically active moiety, or a prodrug form of the biologically active moiety, and forms a stable, non-toxic entity that has no further effect on the activity of the biologically active moiety.

In certain embodiments of Formula (Ia), (Ib), (Ic), (Ib-1), (Ib-2), (Ic-1), or (Ic-2), including any of the foregoing, W¹ is absent. In certain embodiments of Formula (Ia), (Ib), (Ic), (Ib-1), (Ib-2), (Ic-1), or (Ic-2), including any of the foregoing, W⁰¹ is absent. In certain embodiments of Formula (Ia), (Ib), (Ic), (Ib-1), (Ib-2), (Ic-1), or (Ic-2), including any of the foregoing, W¹ and W⁰¹ are independently

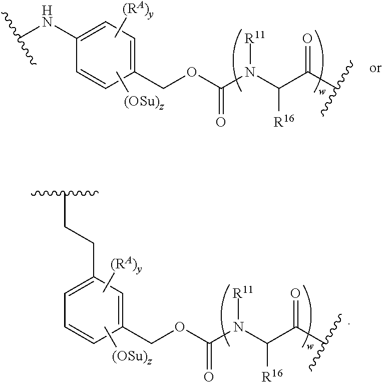

In certain embodiments of (Ia), (Ib), (Ic), (Ib-1), (Ib-2), (Ic-1), or (Ic-2), including any of the foregoing, W¹ and W⁰¹ are independently

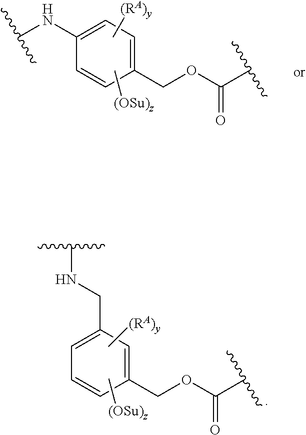

In certain embodiments of Formula (Ia), (Ib), (Ic), (Ib-1), (Ib-2), (Ic-1), or (Ic-2), including any of the foregoing, W¹ and W⁰¹ are independently

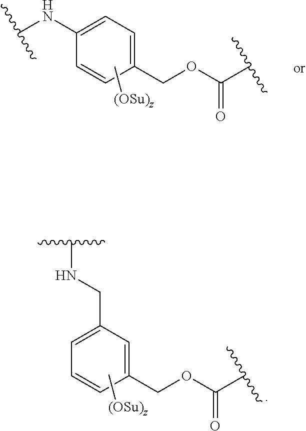

In certain embodiments of Formula (Ia), (Ib), (Ic), (Ib-1), (Ib-2), (Ic-1), or (Ic-2), including any of the foregoing, W¹ and W⁰¹ are independently

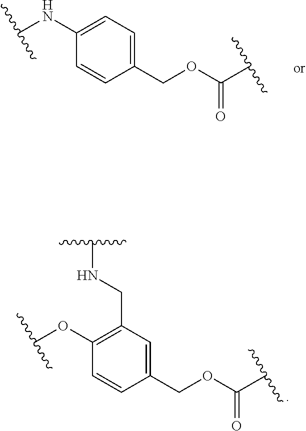 [SMILES: *Nc1ccc(COC(*)=O)cc1 ; *NCc1cc(COC(*)=O)ccc1O*]

In certain embodiments of Formula (Ia), (Ib), (Ic), (Ib-1), (Ib-2), (Ic-1), or (Ic-2), including any of the foregoing, W¹ and W⁰¹ are independently

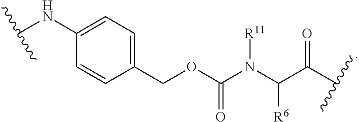

In certain embodiments of Formula (Ia), (Ib), (Ic), (Ib-1), (Ib-2), (Ic-1), or (Ic-2), including any of the foregoing, W¹ and W⁰¹ are independently

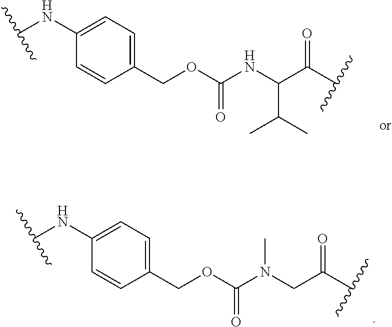 [SMILES: *Nc1ccc(COC(=O)NC(C(*)=O)C(C)C)cc1 ; *Nc1ccc(COC(=O)N(C)CC(*)=O)cc1]

In certain embodiments of Formula (Ia), (Ib), (Ic), (Ib-1), (Ib-2), (Ic-1), or (Ic-2), including any of the foregoing, W¹ and W⁰¹ are independently absent,

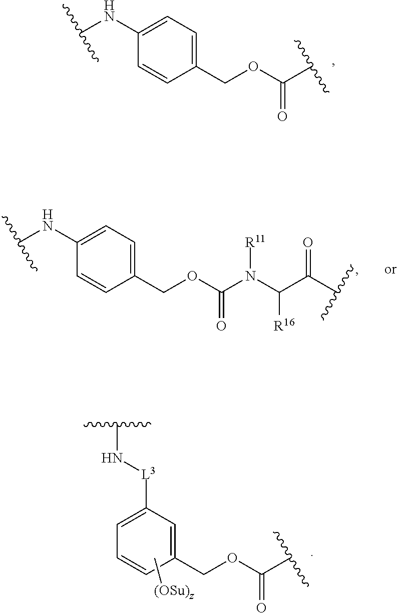

In certain embodiments of Formula (Ia), (Ib), (Ic), (Ib-1), (Ib-2), (Ic-1), or (Ic-2), including any of the foregoing, W¹ and W⁰¹ are independently absent,

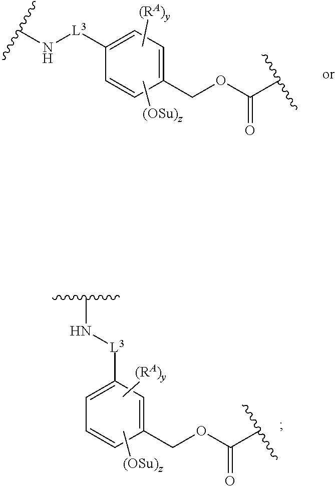

L³ is bond or —CH₂—; y is 0 or 1; and z is 0 or 1.

In certain embodiments of Formula (Ia), (Ib), (Ic), (Ib-1), (Ib-2), (Ic-1), or (Ic-2), including any of the foregoing, y is 0. In certain embodiments of Formula (Ia), (Ib), (Ic), (Ib-1), (Ib-2), (Ic-1), or (Ic-2), including any of the foregoing, z is 0. In certain embodiments of Formula (Ia), (Ib), (Ic), (Ib-1), (Ib-2), (Ic-1), or (Ic-2), including any of the foregoing, z is 1. In certain embodiments of Formula (Ia), (Ib), (Ic), (Ib-1), (Ib-2), (Ic-1), or (Ic-2), including any of the foregoing, OSu is a β-glucuronidase-cleavable β-glucuronide. In certain embodiments of Formula (Ia), (Ib), (Ic), (Ib-1), (Ib-2), (Ic-1), or (Ic-2), including any of the foregoing, OSu is

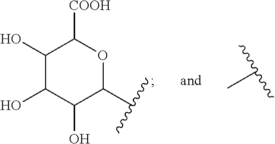

and is the point of attachment to the rest of the formula. In certain embodiments of Formula (Ia), (Ib), (Ic), (Ib-1), (Ib-2), (Ic-1), or (Ic-2), including any of the foregoing, OSu is

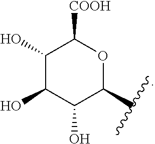 [SMILES: *[C@@H]1O[C@H](C(=O)O)[C@@H](O)[C@H](O)[C@H]1O]

When z is 1, the conjugate comprises a release trigger group, designated as OSu herein, which facilitates the separation of a biologically active portion of a compound or conjugate described herein from the remainder of the compound or conjugate in vivo and/or in vitro. Release trigger groups can also facilitate separation of a biologically active portion of a compound or conjugate described herein in conjunction with an eliminator group. For example, the eliminator group and the release trigger group can react in a Releasing Reaction to release a biologically active portion of a compound or conjugate described herein from the compound or conjugate in vivo and/or in vitro. In certain embodiment, the release trigger can act through a biologically-driven reaction with high tumor:nontumor specificity, such as the proteolytic action of an enzyme overexpressed in a tumor environment.

In certain embodiments of Formula (Ia), (Ib), (Ic), (Ib-1), (Ib-2), (Ic-1), or (Ic-2), W¹ and W⁰¹ are independently

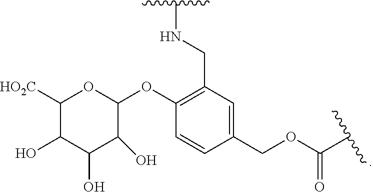 [SMILES: *NCc1cc(COC(*)=O)ccc1OC1OC(C(=O)O)C(O)C(O)C1O]

In certain embodiments of Formula (Ia), (Ib), (Ic), (Ib-1), (Ib-2), (Ic-1), or (Ic-2), W¹ and W⁰¹ are independently

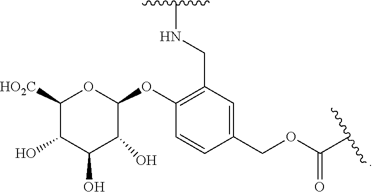 [SMILES: *NCc1cc(COC(*)=O)ccc1O[C@@H]1O[C@H](C(=O)O)[C@@H](O)[C@H](O)[C@H]1O]

Hydrophilic groups, designated HP¹, HP⁰¹ and HP² herein, facilitate increasing the hydrophilicity of the compounds described herein. It is believed that increased hydrophilicity allows for greater solubility in aqueous solutions, such as aqueous solutions found in biological systems. Hydrophilic groups can also function as spacer groups, which are described in further detail herein. Useful hydrophilic groups include those described herein.

In certain embodiments, Formula (Ia), (Ib), (Ic), (Ib-1), (Ib-2), (Ic-1), or (Ic-2), including any of the foregoing, HP¹ and HP⁰¹ are independently a divalent poly(ethylene glycol). In certain embodiments of Formula (Ia), (Ib), (Ic), (Ib-1), (Ib-2), (Ic-1), or (Ic-2), including any of the foregoing, HP¹ and HP⁰¹ are independently absent or

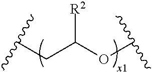

wherein R² is hydrogen or methyl and x1 is an integer between 1 and 50, inclusive; and each [symbol] is the point of attachment to the rest of the formula. In certain embodiments, R² is hydrogen. In certain embodiments, x1 is an integer between 1 and 10, inclusive. In certain embodiments, R² is hydrogen. In certain embodiments, x1 is an integer between 1 and 15, inclusive. In certain embodiments, x1 is 10. In certain embodiments, x1 is an integer between 1 and 5, inclusive. In certain embodiments, x1 is 4. In certain embodiments, x1 is an integer between 1 and 5, inclusive. In certain embodiments, x1 is 13. In certain embodiments of Formula (Ia), (Ib), (Ic), (Ib-1), (Ib-2), (Ic-1), or (Ic-2), including any of the foregoing, HP¹ is absent. In certain embodiments of Formula (Ia), (Ib), (Ic), (Ib-1), (Ib-2), (Ic-1), or (Ic-2), including any of the foregoing, HP⁰¹ is

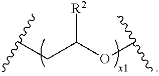

In certain embodiments of Formula (Ia), (Ib), (Ic), (Ib-1), (Ib-2), (Ic-1), or (Ic-2), including any of the foregoing, W² and W⁰² are independently absent or an amino acid residue or a peptide residue wherein the amino acid residue or the peptide residue is optionally substituted with HP². In certain embodiments of Formula (Ia), (Ib), (Ic), (Ib-1), (Ib-2), (Ic-1), or (Ic-2), including any of the foregoing, W² and W⁰² are independently an amino acid residue or a peptide residue wherein the amino acid residue or the peptide residue is optionally substituted with HP². In certain embodiments, the at least one non-natural amino acid is selected from 3-sulfoalanine, hydroxyproline (Hyp), citrulline (Cit), ornithine (Orn), norleucine (Nle), 3-nitrotyrosine, nitroarginine, pyroglutamic acid (Pyr), naphtylalanine (Nal), 2,4-diaminobutyric acid (DAB), methionine sulfoxide, methionine sulfone, 2,3-diaminopropionate, and beta-alanine. In certain embodiments, the at least one non-natural amino acid is citrulline (Cit). In certain embodiments, the at least one non-natural amino acid is 2,3-diaminopropionate.

In certain embodiments of Formula (Ia), (Ib), (Ic), (Ib-1), (Ib-2), (Ic-1), or (Ic-2), including any of the foregoing, W² and W⁰² are independently an amino acid residue. In certain embodiments, the amino acid residue is a beta-amino acid.

In certain embodiments of Formula (Ia), (Ib), (Ic), (Ib-1), (Ib-2), (Ic-1), or (Ic-2), including any of the foregoing, W² and W⁰² are independently selected from —(C(O)CHR¹⁰NR¹¹)ₒ—, —(C(O)CH₂CHR¹⁰NR¹¹)ₚ—, —(C(O)CHR¹⁰CH₂NR¹¹)ₚ—, —(C(O)CH₂CHR¹⁰NR¹¹)ₚ—(C(O)CHR¹⁰NR¹¹)ₒ, —(C(O)CHR¹⁰CH₂NR¹¹)ₚ—(C(O)CHR¹⁰NR¹¹)ₒ—, —(C(O)CHR¹⁰NR¹¹)ₒ—(C(O)CH₂CHR¹⁰NR¹¹)ₚ—, and —(C(O)CHR¹⁰NR¹¹)ₒ—(C(O)CHR¹⁰CH₂NR¹¹)ₒ—;
wherein R¹⁰ is independently an amino acid sidechain residue optionally substituted with HP²;
R¹¹ is independently hydrogen or C₁₋₆ alkyl;
and p is an integer independently an integer between 1 and 10, inclusive; and the —C(O)— of W² or W⁰² is bound to W¹ or W⁰¹, respectively.

In certain embodiments of Formula (Ia), (Ib), (Ic), (Ib-1), (Ib-2), (Ic-1), or (Ic-2), including any of the foregoing, W² and W⁰² are independently selected from —(C(O)CHR¹⁰NR¹¹)ₒ—, —(C(O)CH₂CHR¹⁰NR¹¹)ₚ—, —(C(O)CHR¹⁰CH₂NR¹¹)ₚ—, —(C(O)CH₂CHR¹⁰NR¹¹)ₚ—(C(O)CHR¹⁰NR¹¹)ₒ—, and —(C(O)CHR¹⁰CH₂NR¹¹)ₚ—(C(O)CHR¹⁰NR¹¹)ₒ—.

In certain embodiments of Formula (Ia), (Ib), (Ic), (Ib-1), (Ib-2), (Ic-1), or (Ic-2), including any of the foregoing, W² or W⁰² is —(C(O)CHR¹⁰NR¹¹)ₒ—. In certain embodiments of Formula (Ia), (Ib), (Ic), (Ib-1), (Ib-2), (Ic-1), or (Ic-2), including any of the foregoing, W² or W⁰² is —(C(O)CH₂CHR¹⁰NR¹¹)ₚ—. In certain embodiments of Formula (Ia), (Ib), (Ic), (Ib-1), (Ib-2), (Ic-1), or (Ic-2), including any of the foregoing, W² or W⁰² is —(C(O)CH₂CHR¹⁰NR¹¹)ₒ—(C(O)CHR¹⁰NR¹¹)ₒ—.

In certain embodiments, including any of the foregoing, R¹⁰ is an amino acid sidechain residue independently selected from valine, citrulline, alanine, glycine, leucine, asparagine, lysine and 2,3-diaminopropionate optionally substituted with HP². In certain embodiments, including any of the foregoing, R¹¹ is hydrogen. In certain embodiments, including any of the foregoing, o is independently an integer between 1 and 4, inclusive. In certain embodiments, including any of the foregoing, p is 1.

In certain embodiments, including any of the foregoing, R¹⁰ is an amino acid sidechain residue independently selected from valine, citrulline, alanine, glycine, and 2,3-diaminopropionate optionally substituted with HP². In certain embodiments, including any of the foregoing, R¹¹ is hydrogen. In certain embodiments, including any of the foregoing, o is independently an integer between 1 and 4, inclusive. In certain embodiments, including any of the foregoing, p is 1.

In certain embodiments of Formula (Ia), (Ib), (Ic), (Ib-1), (Ib-2), (Ic-1), or (Ic-2), including any of the foregoing, W² and W⁰² are independently absent or —(C(O)CH₂CHR¹⁰NR¹¹)ₚ—(C(O)CHR¹⁰NR¹¹)ₒ—;
wherein
R¹⁰ is an amino acid sidechain residue independently selected from valine, citrulline, alanine, glycine, leucine, asparagine, lysine and 2,3-diaminopropionate and wherein the amino acid sidechain is optionally substituted with HP²;
HP² is independently polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), polysarcosine, or a combination thereof,
R¹¹ is hydrogen;
is independently an integer between 1 and 4, inclusive; and
p is 1.

In certain embodiments of Formula (Ia), (Ib), (Ic), (Ib-1), (Ib-2), (Ic-1), or (Ic-2), including any of the foregoing, W² and W⁰² are independently absent or —(C(O)CH₂CHR¹⁰NR¹¹)ₚ—(C(O)CHR¹⁰NR¹¹)ₒ—;
wherein
R¹⁰ is an amino acid sidechain residue independently selected from valine, citrulline, alanine, glycine, and 2,3-diaminopropionate and wherein the amino acid sidechain is optionally substituted with HP²;
HP² is independently polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), polysarcosine, or a combination thereof,
R¹¹ is hydrogen;
is independently an integer between 1 and 4, inclusive; and
p is 1.

In certain embodiments of Formula (Ia), (Ib), (Ic), (Ib-1), (Ib-2), (Ic-1), or (Ic-2), including any of the foregoing, W² and W⁰² are independently absent or of the formula:

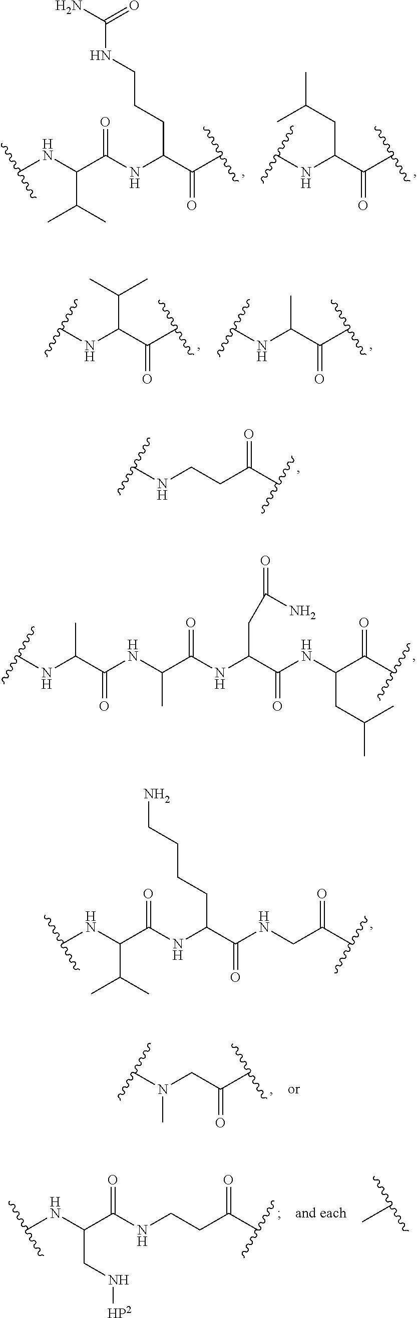

is the point of attachment to the rest of the formula. In certain embodiments of Formula (Ia), (Ib), (Ic), (Ib-1), (Ib-2), (Ic-1), or (Ic-2), including any of the foregoing, W² and W⁰² are independently of the formula:

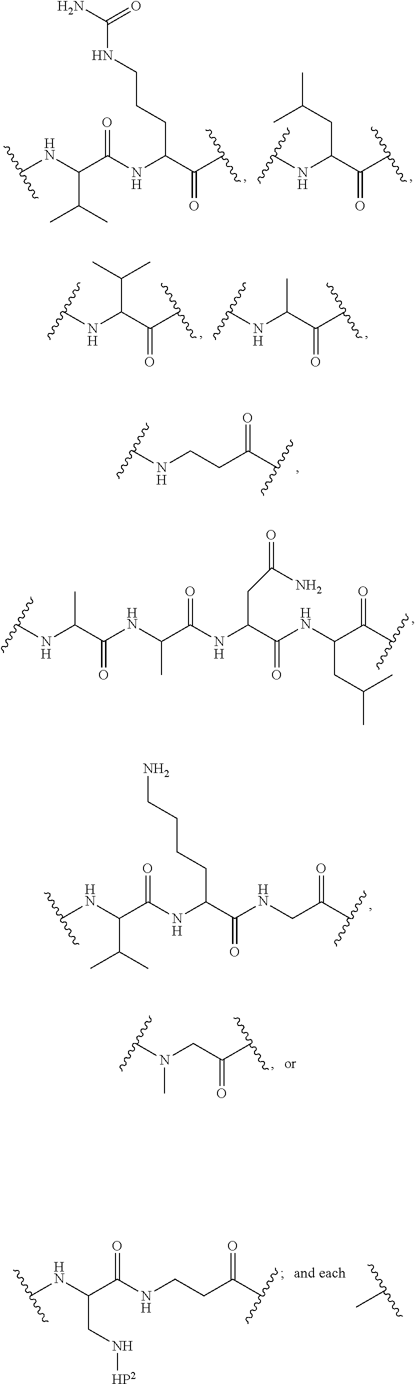

is the point of attachment to the rest of the formula. In certain embodiments of Formula (Ia), (Ib), (Ic), (Ib-1), (Ib-2), (Ic-1), or (Ic-2), including any of the foregoing, W² and W⁰² are independently of the formula:

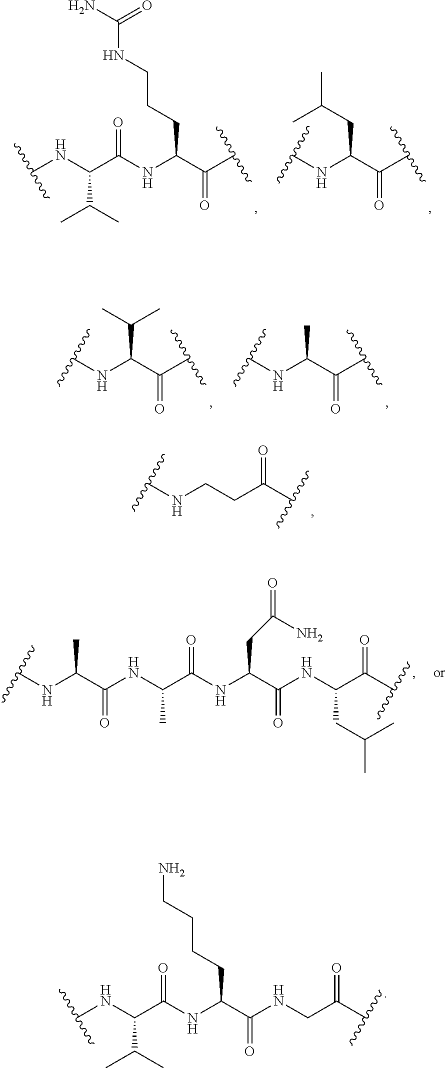

In certain embodiments of Formula (Ia), (Ib), (Ic), (Ib-1), (Ib-2), (Ic-1), or (Ic-2), including any of the foregoing, W² or W⁰² is

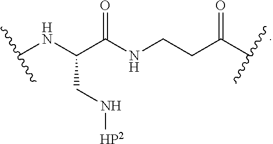

In certain embodiments of Formula (Ia), (Ib), (Ic), (Ib-1), (Ib-2), (Ic-1), or (Ic-2), including any of the foregoing, W² or W⁰² is

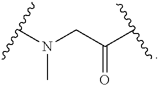 [SMILES: *C(=O)CN(*)C]

In certain embodiments of Formula (Ia), (Ib), (Ic), (Ib-1), (Ib-2), (Ic-1), or (Ic-2), including any of the foregoing, HP² is independently

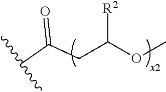

wherein R² is hydrogen or methyl and x2 is an integer between 1 and 50, inclusive; and each [symbol] is the point of attachment to the rest of the formula. In certain embodiments, x2 is independently an integer between 10 and 20, inclusive. In certain embodiments, x2 is 12. In certain embodiments of Formula (Ia), (Ib), (Ic), (Ib-1), (Ib-2), (Ic-1), or (Ic-2), including any of the foregoing, W² or W⁰² is absent.

In certain embodiments of Formula (Ia), (Ib), (Ic), (Ib-1), (Ib-2), (Ic-1), or (Ic-2), -W¹-W²- or -W⁰¹-W⁰²- is independently selected from

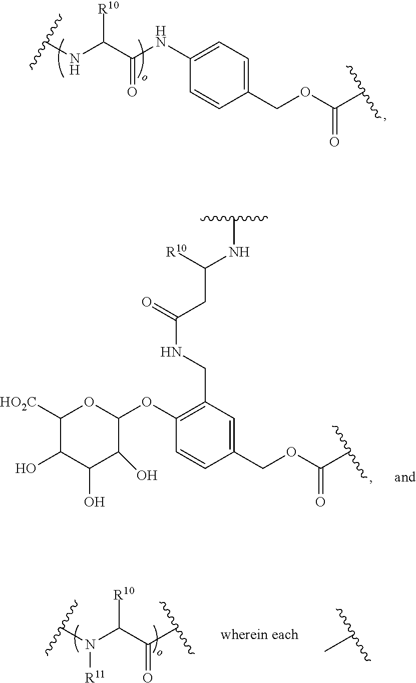

is the point of attachment to the rest of the formula. In certain embodiments of Formula (Ia), (Ib), (Ic), (Ib-1), (Ib-2), (Ic-1), or (Ic-2), -W¹-W²- or -W⁰¹-W⁰²- is independently

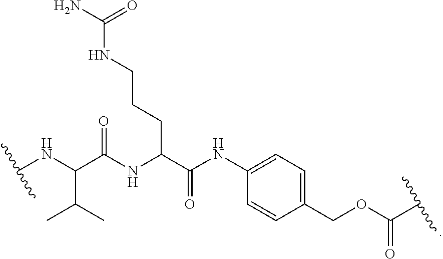 [SMILES: *NC(C(=O)NC(CCCNC(N)=O)C(=O)Nc1ccc(COC(*)=O)cc1)C(C)C]

In certain embodiments of Formula (Ia), (Ib), (Ic), (Ib-1), (Ib-2), (Ic-1), or (Ic-2), -W¹-W²- or -W⁰¹-W⁰²- is independently

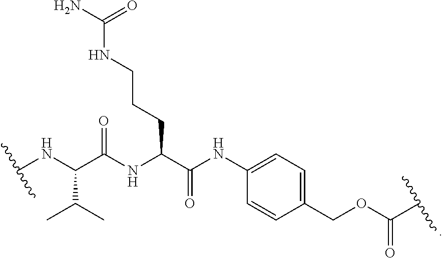 [SMILES: *N[C@H](C(=O)N[C@@H](CCCNC(N)=O)C(=O)Nc1ccc(COC(*)=O)cc1)C(C)C]

In certain embodiments of Formula (Ia), (Ib), (Ic), (Ib-1), (Ib-2), (Ic-1), or (Ic-2), -W¹-W²- or -W⁰¹-W⁰²- is independently

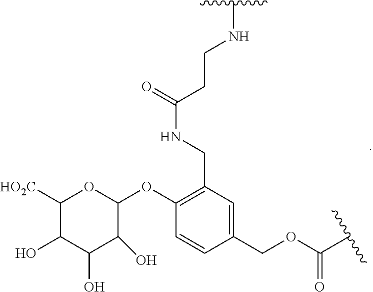 [SMILES: *NCCC(=O)NCc1cc(COC(*)=O)ccc1OC1OC(C(=O)O)C(O)C(O)C1O]

In certain embodiments of Formula (Ia), (Ib), (Ic), (Ib-1), (Ib-2), (Ic-1), or (Ic-2), -W¹-W²- or -W⁰¹-W⁰²- is independently

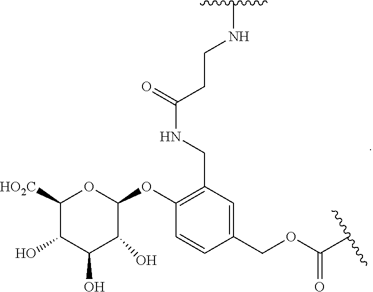 [SMILES: *NCCC(=O)NCc1cc(COC(*)=O)ccc1O[C@@H]1O[C@H](C(=O)O)[C@@H](O)[C@H](O)[C@H]1O]

In certain embodiments of Formula (Ia), (Ib), (Ic), (Ib-1), (Ib-2), (Ic-1), or (Ic-2), -W¹-W²- or -W⁰¹-W⁰²- is independently

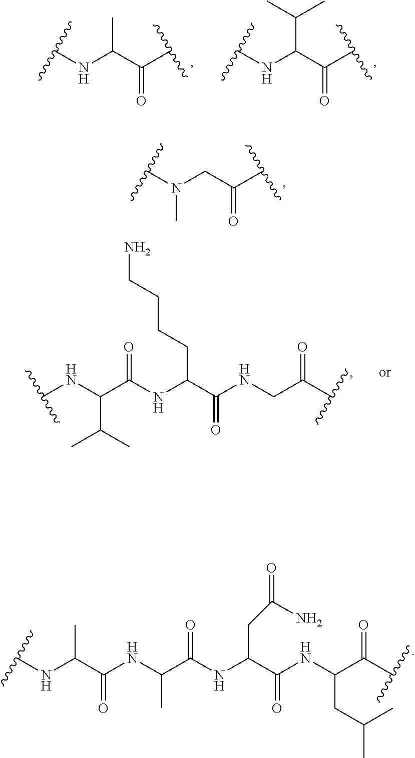

In certain embodiments of Formula (Ia), (Ib), (Ic), (Ib-1), (Ib-2), (Ic-1), or (Ic-2), -W¹—W²— or -W⁰¹-W⁰²- is independently

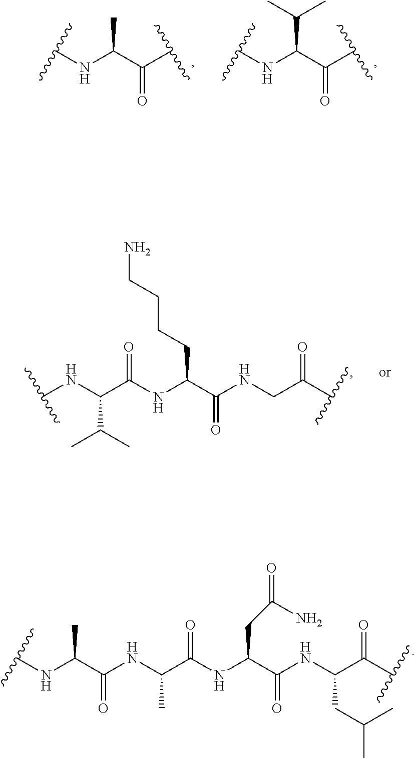

In certain embodiments of Formula (Ib), (Ic), (Ib-1), (Ib-2), (Ic-1), or (Ic-2), including any of the foregoing, Y is —X¹—C₁₋₄ alkylene-X¹—C₁₋₄ alkylene-X¹—C₁₋₄ alkylene-X¹—, wherein at least one C₁₋₄ alkylene in Y is substituted with one or more substituents selected from R⁷⁰. In certain embodiments of Formula (Ib), (Ic), (Ib-1), (Ib-2), (Ic-1), or (Ic-2), including any of the foregoing, Y is —X¹—C₁₋₄ alkylene-X¹—C₁₋₄ alkylene-X¹—, wherein at least one C₁₋₄ alkylene in Y is substituted with one or more substituents selected from R⁷⁰; and —[X¹]_{b}— is attached to RL¹ or RL⁰¹.

In certain embodiments of Formula (Ib), (Ic), (Ib-1), (Ib-2), (Ic-1), or (Ic-2), including any of the foregoing, R⁷⁰ is —C₁₋₆ alkylene-X²—[C₁₋₆ alkylene]_{c}-HP², wherein each alkylene of R⁷⁰ is optionally substituted with one or more substituents selected from halogen, —CN, —NO₂, —OH, —NR⁶⁰R⁶¹, —C(O)N NR⁶⁰R⁶¹, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl.

In certain embodiments of Formula (Ib), (Ic), (Ib-1), (Ib-2), (Ic-1), or (Ic-2), including any of the foregoing, c is 1.

In certain embodiments of Formula (Ib), (Ic), (Ib-1), (Ib-2), (Ic-1), or (Ic-2), including any of the foregoing, HP² is polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), polysarcosine, or a combination thereof. In certain embodiments of Formula (Ib), (Ic), (Ib-1), (Ib-2), (Ic-1), or (Ic-2), including any of the foregoing, HP² is a polyethylene glycol (PEG) or methoxypolyethylene glycol (mPEG). In certain embodiments of Formula (Ib), (Ic), (Ib-1), (Ib-2), (Ic-1), or (Ic-2), including any of the foregoing, HP² is independently

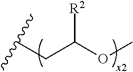

wherein R² is hydrogen or methyl and x2 is an integer between 1 and 50, inclusive; and each [symbol] is the point of attachment to the rest of the formula. In certain embodiments, x2 is independently an integer between 10 and 20, inclusive. In certain embodiments, x2 is 12.

In certain embodiments of Formula (Ib), (Ic), (Ib-1), (Ib-2), (Ic-1), or (Ic-2), including any of the foregoing, RL¹ and RL^{O1} are independently selected from

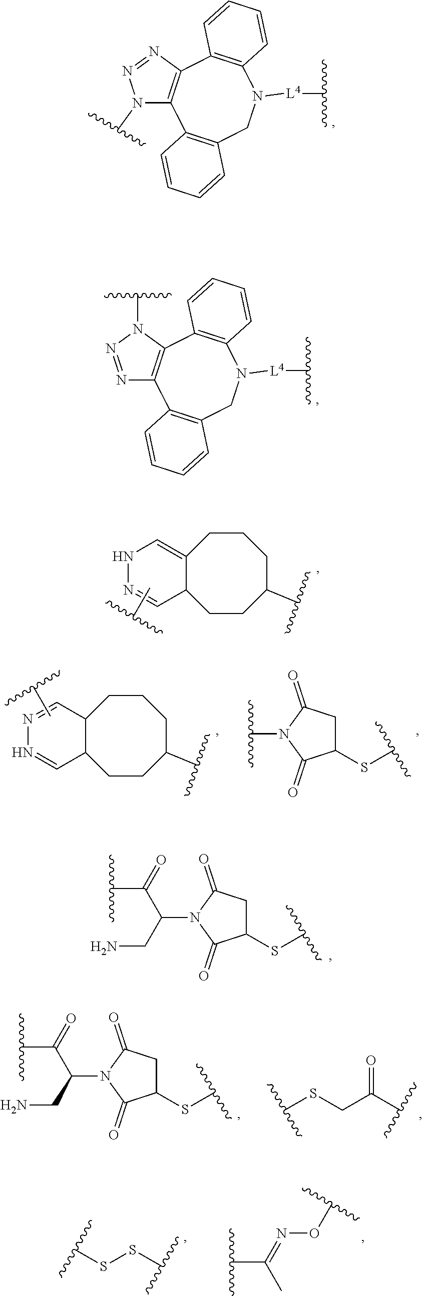

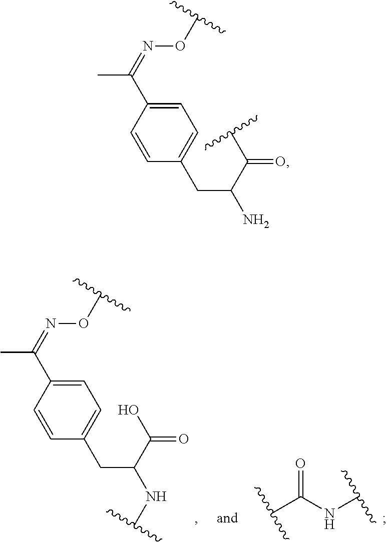

wherein L⁴ is bond, —C(O)C₁₋₆alkylene-C(O)—, —C(O)C₁₋₆alkylene-NR¹¹C(O)—,

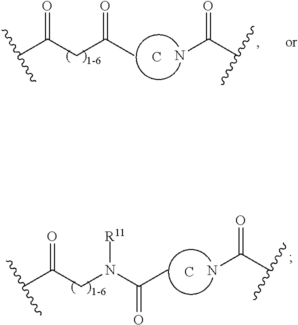

Ring C is an optionally substituted N-linked bridged, fused, or spirocyclic bicyclic heterocycle; and
each [symbol] is a point of attachment to the rest of the formula.

In certain embodiments, L⁴ is a bond. In certain embodiments, L⁴ is —C(O)C₁₋₆alkylene-NR¹¹C(O)—. In certain embodiments, L⁴ is

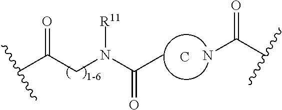

wherein Ring C is a spirocyclic bicyclic heterocycle.

In certain embodiments of Formula (Ib), (Ic), (Ib-1), (Ib-2), (Ic-1), or (Ic-2), including any of the foregoing, RL¹ is

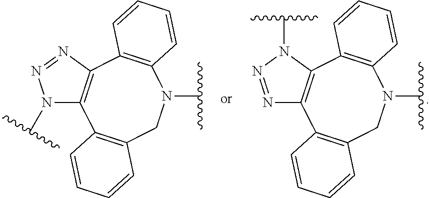 [SMILES: *N1Cc2ccccc2-c2c(nnn2*)-c2ccccc21 ; *N1Cc2ccccc2-c2nnn(*)c2-c2ccccc21]

In certain embodiments of Formula (Ib), (Ic), (Ib-1), (Ib-2), (Ic-1), or (Ic-2), including any of the foregoing, RL¹ is

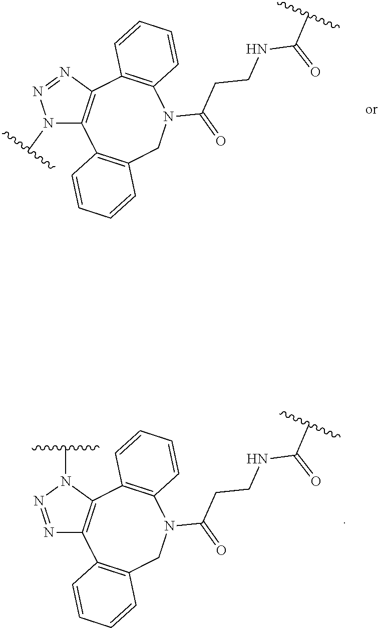 [SMILES: *C(=O)NCCC(=O)N1Cc2ccccc2-c2c(nnn2*)-c2ccccc21 ; *C(=O)NCCC(=O)N1Cc2ccccc2-c2nnn(*)c2-c2ccccc21]

In certain embodiments of Formula (Ib), (Ic), (Ib-1), (Ib-2), (Ic-1), or (Ic-2), including any of the foregoing, RL⁰¹ is

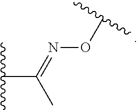 [SMILES: *O/N=C(/*)C]

In certain embodiments of Formula (Ia), (Ic), (Ic-1), or (Ic-2),

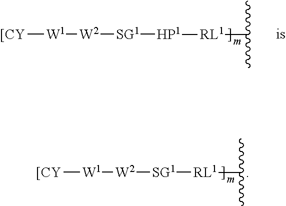

In certain embodiments of Formula (Ia), (Ic), (Ic-1), or (Ic-2),

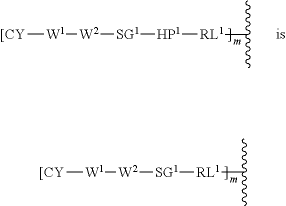

wherein RL¹ is

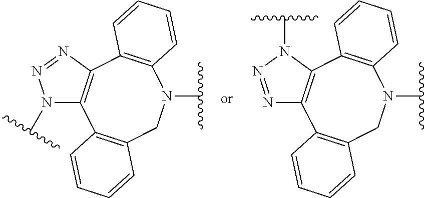 [SMILES: *N1Cc2ccccc2-c2c(nnn2*)-c2ccccc21 ; *N1Cc2ccccc2-c2nnn(*)c2-c2ccccc21]

and SG¹ is —C(O)C₁₋₆alkylene-C(O)—. In certain embodiments of Formula (Ia), (Ic), (Ic-1), or (Ic-2),

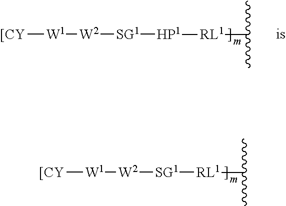

wherein RL¹ is

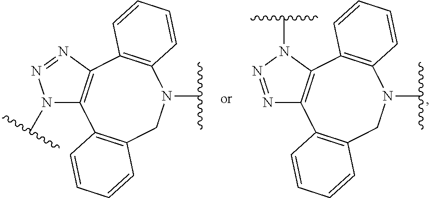 [SMILES: *N1Cc2ccccc2-c2c(nnn2*)-c2ccccc21 ; *N1Cc2ccccc2-c2nnn(*)c2-c2ccccc21]

SG¹ is —C(O)C₁₋₆alkylene-C(O)—, and W¹ is

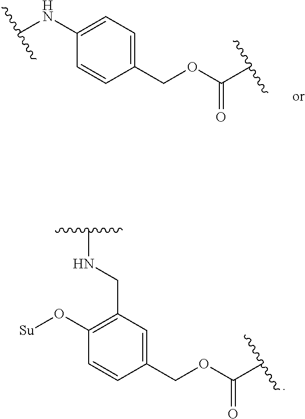

In certain embodiments of Formula (Ib), (Ib-1), or (Ib-2), -SG¹-Y-RL¹- is —CH₂—Y—C(O)(C₁₋₆ alkylene)OC(O)—. In certain embodiments of Formula (Ib), (Ib-1), or (Ib-2), -SG¹-Y-RL¹- is —CH₂—X¹—C₁₋₆ alkylene-[X¹—C₁₋₆ alkylene]ₐ-[X¹]_{b}—C(O)(C₁₋₆ alkylene)OC(O)—. In certain embodiments of Formula (Ib), (Ib-1), or (Ib-2), -SG¹-Y-RL¹- is —CH₂—NHC(O)—C₁₋₆ alkylene-NHC(O)—C₁₋₆ alkylene-NH—C(O)(C₁₋₆ alkylene)OC(O)—.

In certain embodiments of Formula (Ib) or (Ib-2),

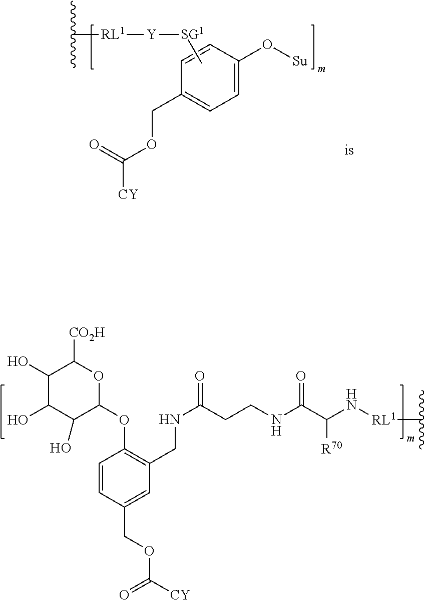

In certain embodiments of Formula (Ia), (Ib), (Ib-1), or (Ib-2),

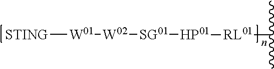

is selected from

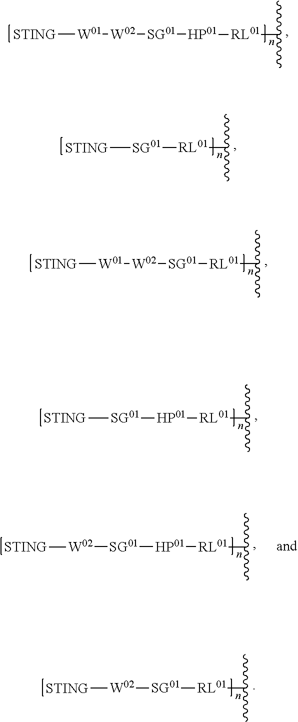

In certain embodiments of Formula (Ia), (Ib), (Ib-1), or (Ib-2),

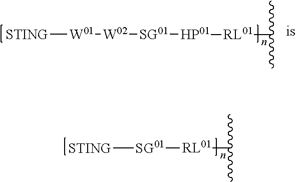

and SG⁰¹-RL⁰¹- is —C(O)CH₂—O—N═C(CH₃)— or —C(O)(CH₂)₅—O—N═C(CH₃)—.

In certain embodiments of Formula (Ia), (Ib), (Ib-1), or (Ib-2),

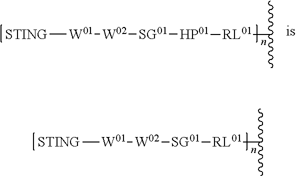

wherein W⁰¹ is

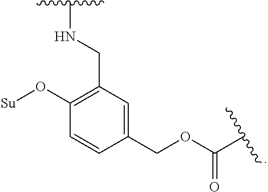

W⁰² is a beta-amino acid, SG⁰¹ is —C(O)C₁₋₆alkylene, and RL⁰¹ is

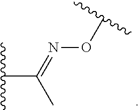 [SMILES: *O/N=C(/*)C]

In certain embodiments of Formula (Ia), (Ib), (Ib-1), or (Ib-2),

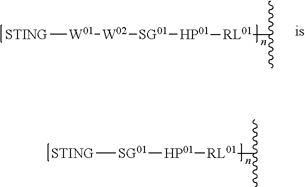

and SG⁰¹-HP⁰¹-RL⁰¹- is —C(O)CH₂CH₂—(OCH₂CH₂)x₁-O—N═C(CH₃)—.

In certain embodiments of Formula (Ia), (Ib), (Ib-1), or (Ib-2),

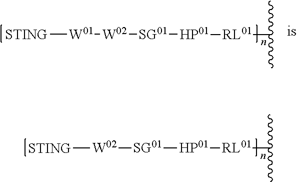

and SG⁰¹-HP⁰¹-RL⁰¹- is —C(O)CH₂CH₂—(OCH₂CH₂)x₁-O—N═C(CH₃)—. In certain embodiments of Formula (Ia), (Ib), (Ib-1), or (Ib-2),

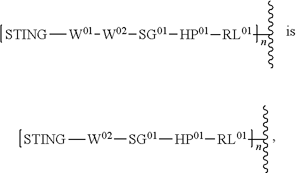

W⁰² is —(C(O)CHR¹⁰NR¹¹)ₒ—, and SG⁰¹-HP⁰¹-RL⁰¹- is —C(O)CH₂CH₂—(OCH₂CH₂)x₁-O—N═C(CH₃)—.

In certain embodiments of Formula (Ia), (Ib), (Ib-1), or (Ib-2),

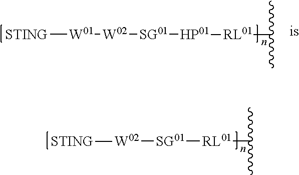

wherein W⁰² is —C(O)CHR¹⁰NR¹¹—, SG⁰¹ is —C(O)C₁₋₆alkylene, and RL⁰¹ is

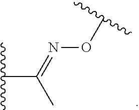 [SMILES: *O/N=C(/*)C]

In certain embodiments of Formula (Ia), (Ib), (Ib-1), or (Ib-2),

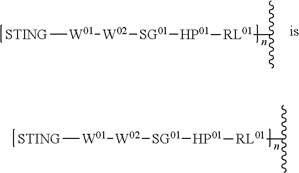

wherein W⁰¹ is

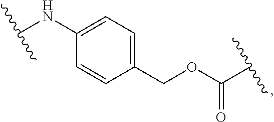 [SMILES: *Nc1ccc(COC(*)=O)cc1]

W⁰² is —(C(O)CHR¹⁰NR¹¹)ₒ—, and SG⁰¹-HP⁰¹-RL⁰¹- is —C(O)CH₂CH₂—(OCH₂CH₂)x₁-O—N═C(CH₃)—. In certain embodiments of Formula (Ia), (Ib), (Ib-1), or (Ib-2),

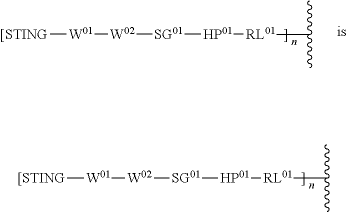

wherein W⁰¹ is

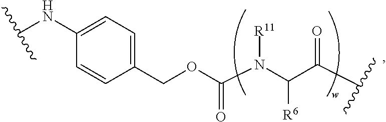

W⁰² is —(C(O)CHR¹⁰NR¹¹)ₒ—, and SG⁰¹-HP⁰¹-RL⁰¹- is —C(O)CH₂CH₂—(OCH₂CH₂)x₁-O—N═C(CH₃)—.

### 2. Cytotoxic Agent (CY)

In some embodiments of Formula (I), (Ia), (Ib), (Ic), (Ib-1), (Ib-2), (Ic-1), or (Ic-2), CY is a cytotoxic agent. Any cytotoxic agent can be used as far as the agent has cytotoxic activity. For example, CY is selected from the group consisting of irinotecan, SN-38, topotecan, exatecan, etoposide, teniposide, tafluposide, hemiasterlins, 3-aminopheny hemiasterlin, taltobulin (HTI-286) amanitin, deruxtecan (DXd), camptothecins, PNU-159682, pyrrolobenzodiazepine (PBD), EDA PNU-159682 derivatives, MMAF, MMAE, daunorubicin, anthracycline, doxorubicin, epirubicin, idarubicin, mitoxantrone, valrubicinm, mitoxantrone, valrubicin, cyclophosphamide, mechlorethamine, chlorambucil, melphalan, vorinostat, romidepsin, bortezomib, erlotinib, gefitinib, imatinib, vemurafenib, vismodegib, vemurafenib, azacitidine, azathioprine, capecitabine, cyatarabine, doxifluridine, fluorouracil, gemcitabine, hydroxyurea, mercaptopurine, methotrexate, and tioguanine (formerly thioguanine), doxifluridine, gemcitabine, methotrexate, carboplatin, cisplatin, and oxaliplatin.

In certain embodiments, CY is selected from the group consisting of an alkylating agent, a DNA-crosslinking agent, an anti-tumor antibiotic, an anti-metabolite, an anti-mitotic agent, a histone-deacetylase (HDAC) inhibitor, a telomerase inhibitor, an immunogenic cell death agent, a tubulin inhibitor, and a topoisomerase inhibitor. In certain embodiments, CY is selected from the group consisting of topoisomerase I inhibitor selected from the group consisting of camptothecin, irinotecan, SN-38, topotecan, and exatecan. In certain embodiments, CY is exatecan.

In some embodiments of Formula (I), (Ia), (Ib), (Ic), (Ib-1), (Ib-2), (Ic-1), or (Ic-2), CY is independently selected from

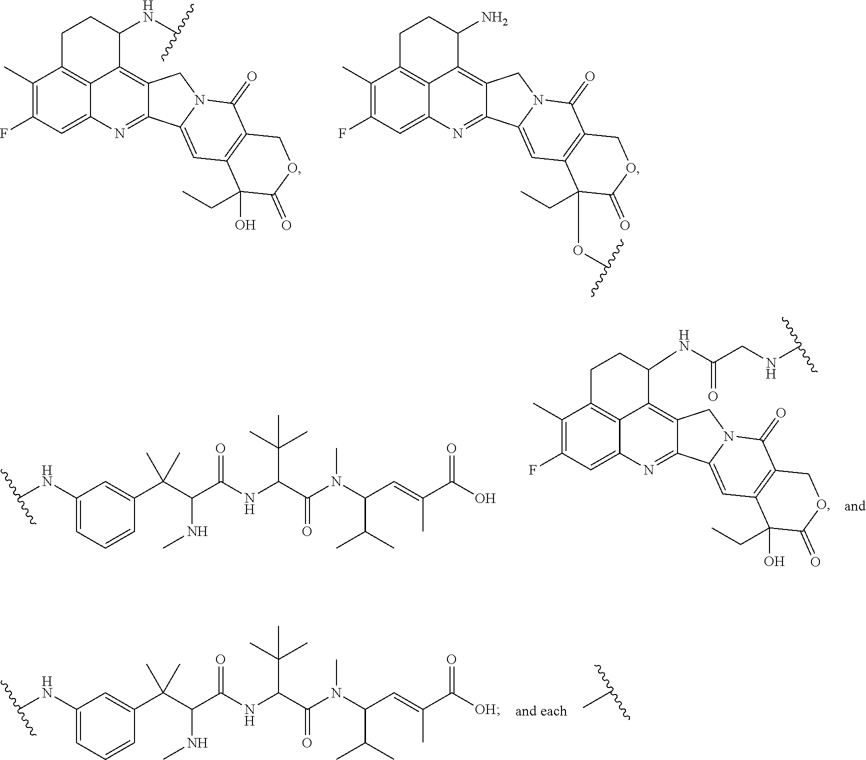

is a point of attachment to the rest of the formula.

In some embodiments of Formula (I), (Ia), (Ib), (Ic), (Ib-1), (Ib-2), (Ic-1), or (Ic-2), CY is independently selected from

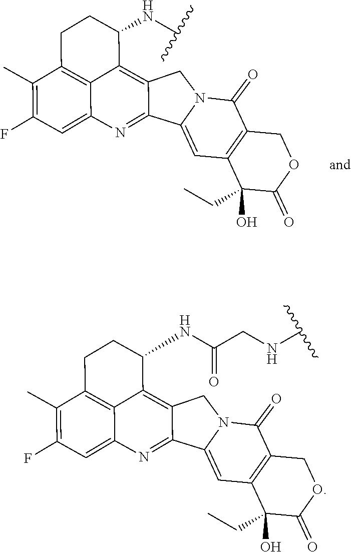 [SMILES: *N[C@H]1CCc2c(C)c(F)cc3nc4c(c1c23)Cn1c-4cc2c(c1=O)COC(=O)[C@]2(O)CC ; "and" ; *NCC(=O)N[C@H]1CCc2c(C)c(F)cc3nc4c(c1c23)Cn1c-4cc2c(c1=O)COC(=O)[C@]2(O)CC]

In some embodiments of Formula (I), (Ia), (Ib), (Ic), (Ib-1), (Ib-2), (Ic-1), or (Ic-2), CY is independently selected from

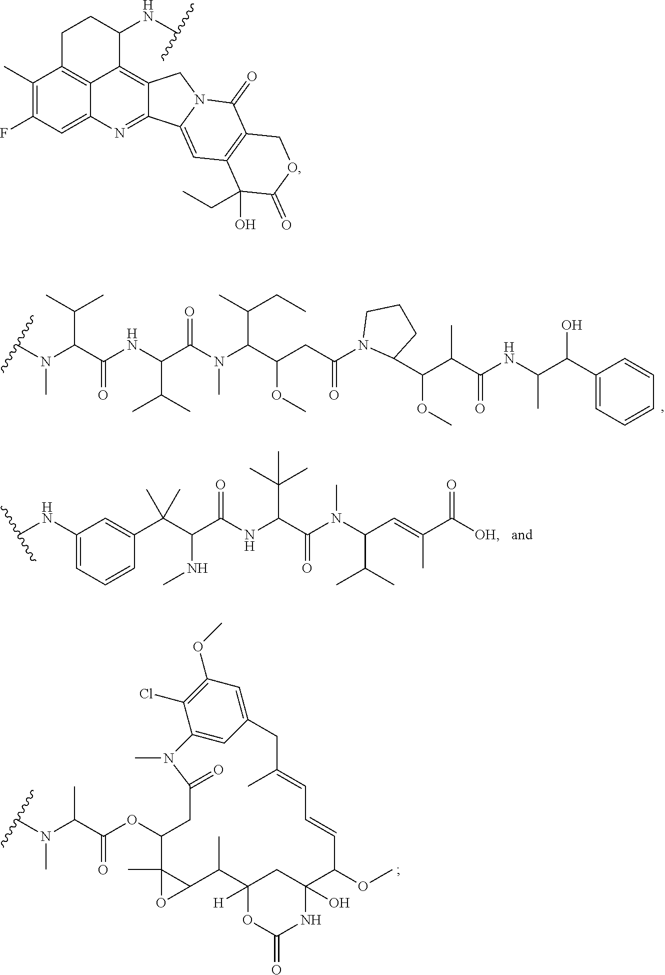 [SMILES: *NC1CCc2c(C)c(F)cc3nc4c(c1c23)Cn1c-4cc2c(c1=O)COC(=O)C2(O)CC ; *N(C)C(C(=O)NC(C(=O)N(C)C(C(C)CC)C(CC(=O)N1CCCC1C(OC)C(C)C(=O)NC(C)C(O)c1ccccc1)OC)C(C)C)C(C)C ; *Nc1cccc(C(C)(C)C(NC)C(=O)NC(C(=O)N(C)C(/C=C(\C)C(=O)O)C(C)C)C(C)(C)C)c1 ; *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

or a derivative thereof, and each [symbol] is a point of attachment to the rest of the formula. In some embodiments of Formula (I), (Ia), (Ib), (Ic), (Ib-1), (Ib-2), (Ic-1), or (Ic-2), CY is independently selected from

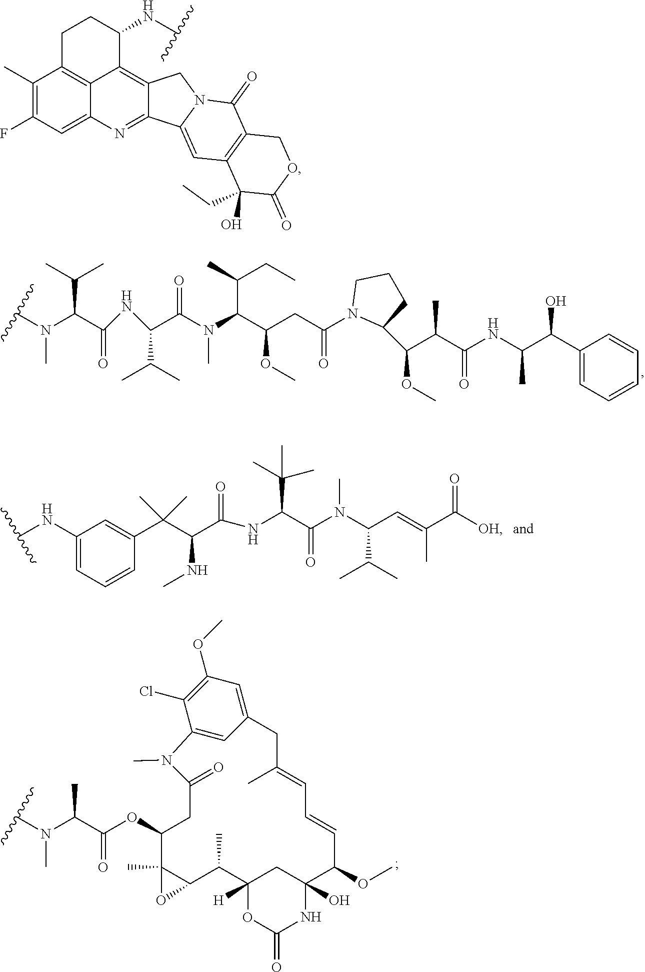

or a derivative thereof.

In certain embodiments of Formula (Ia), (Ic), (Ic-1), or (Ic-2),

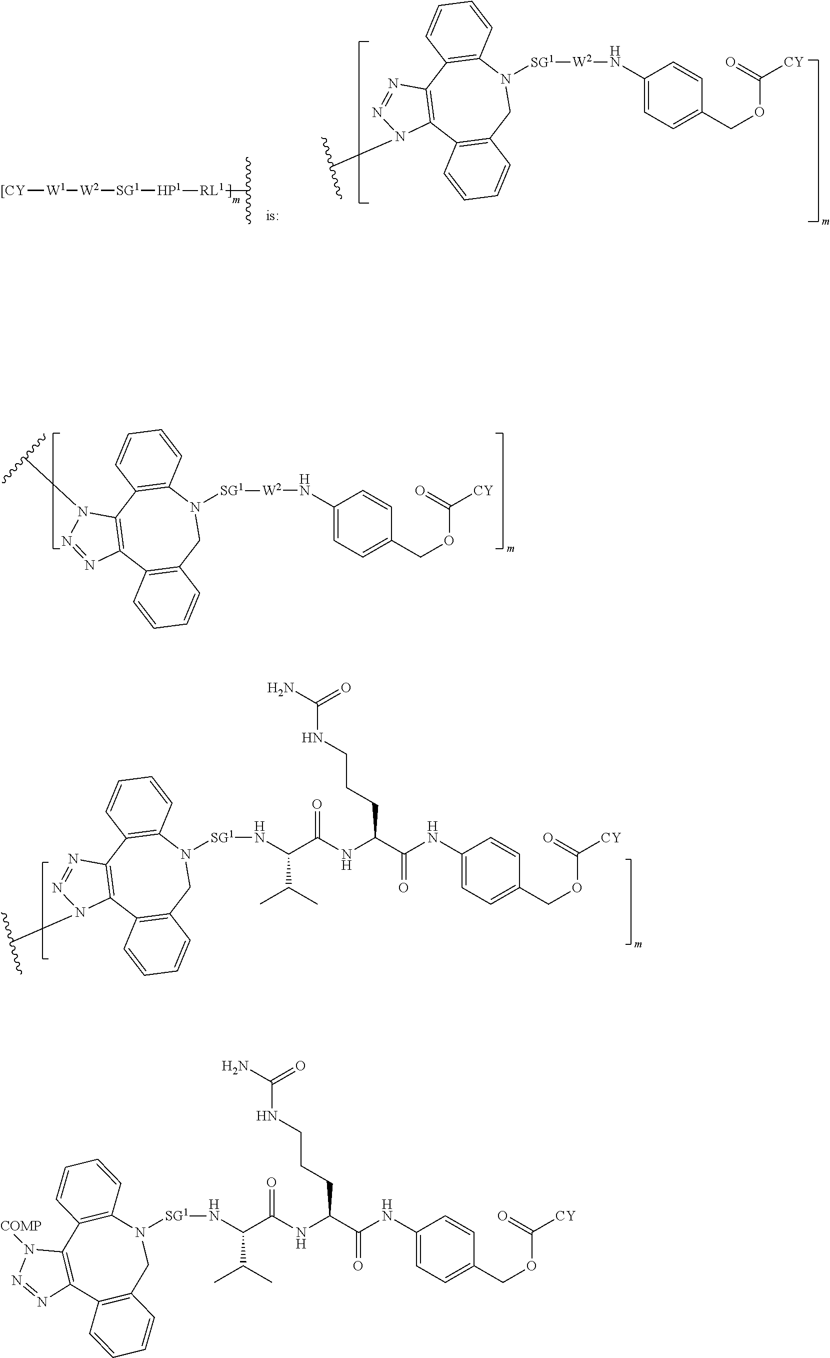

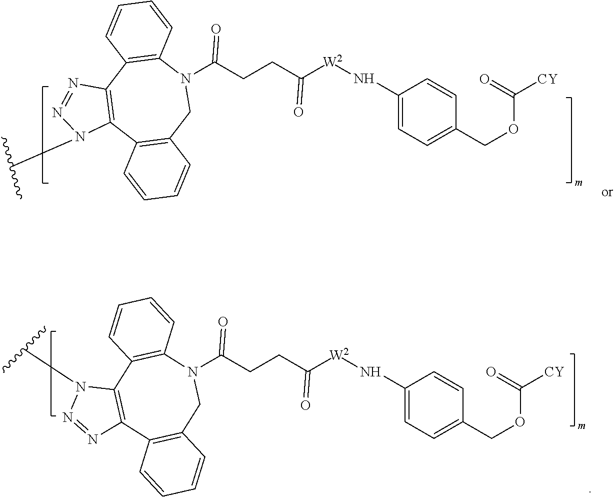

In certain embodiments of Formula (Ia), (Ic), (Ic-1), or (Ic-2),

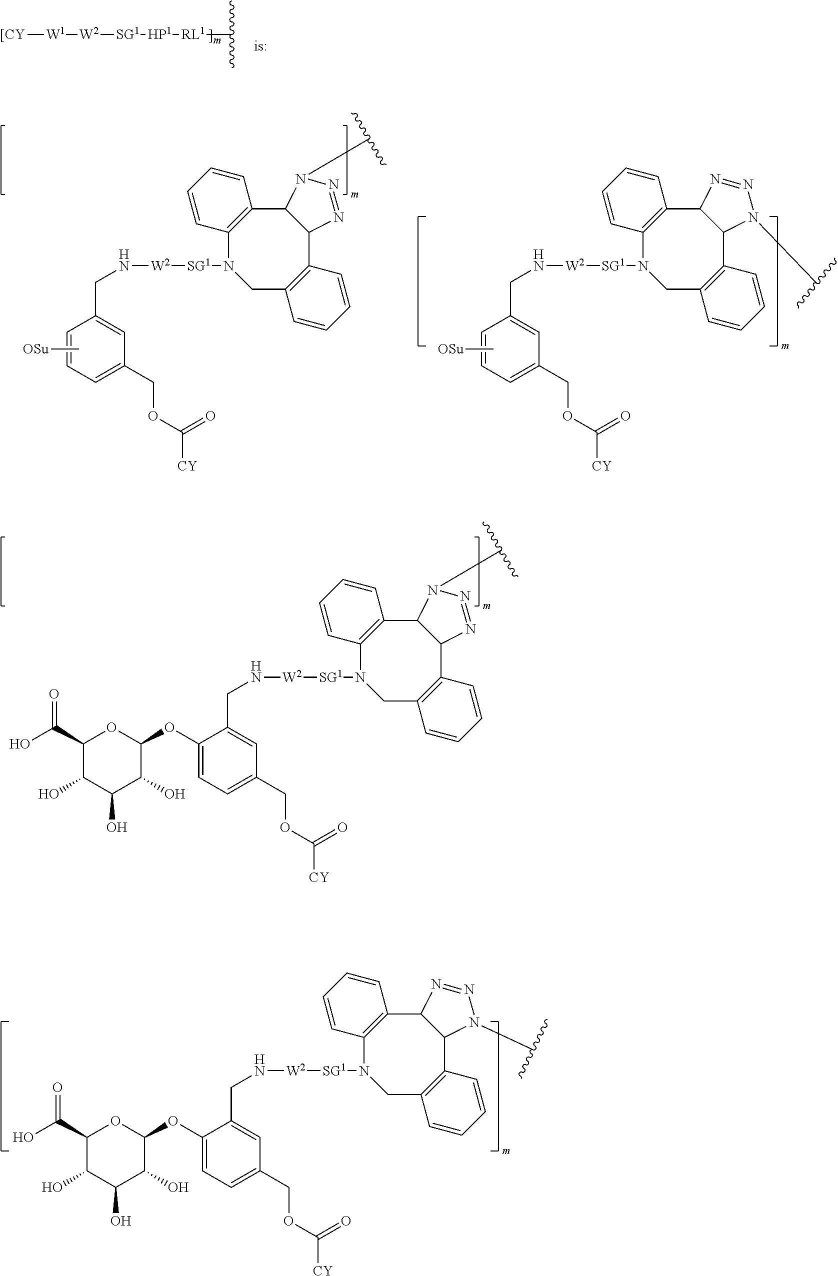

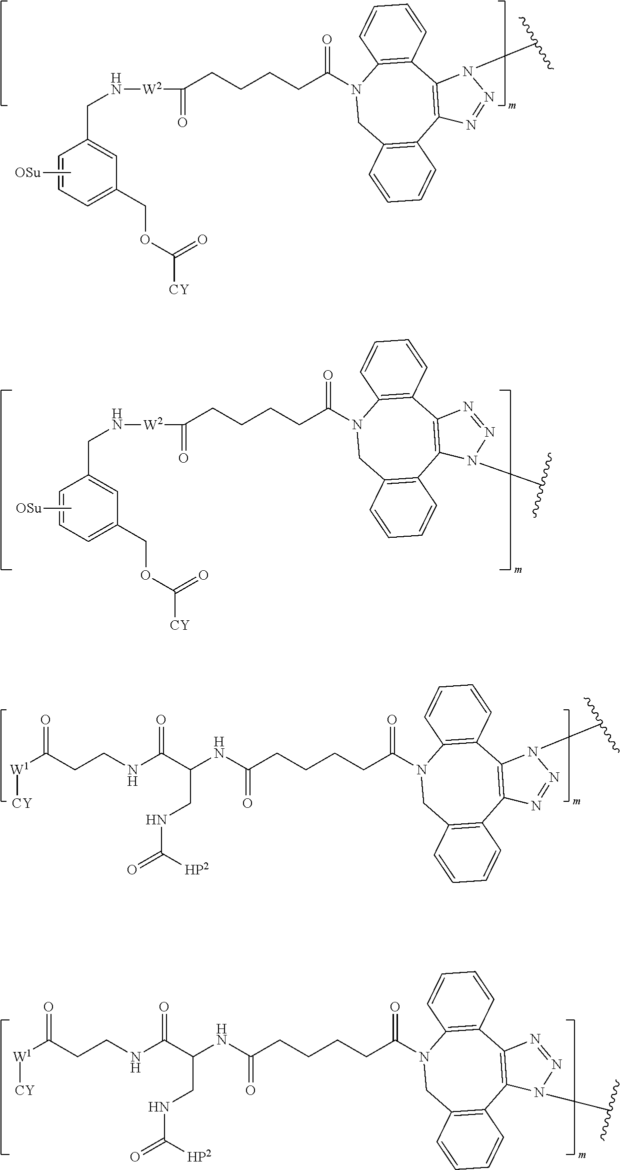

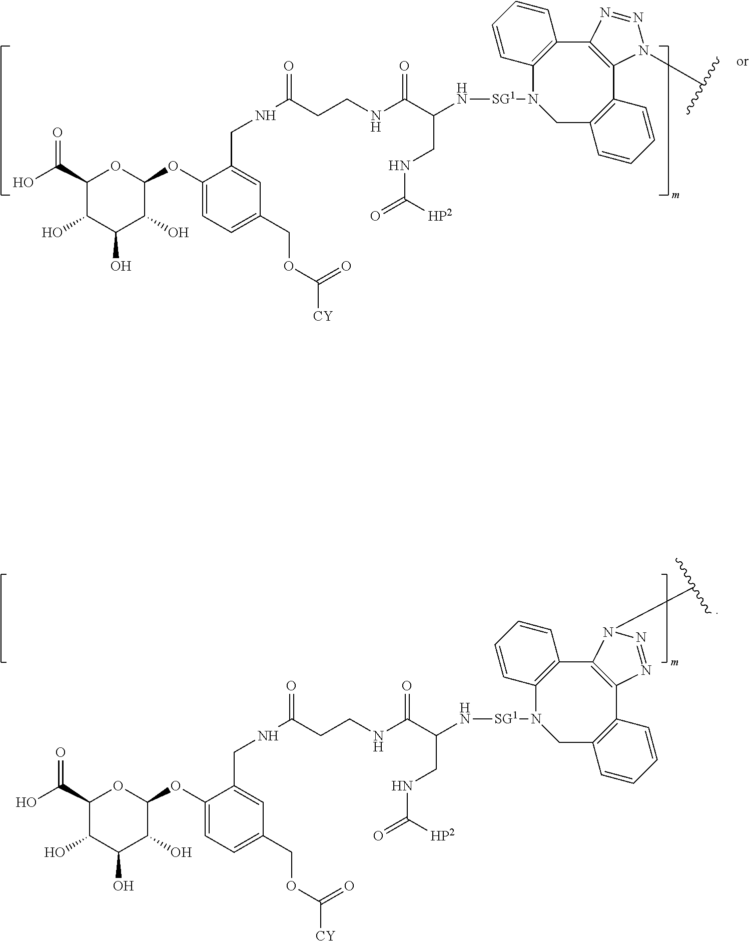

In certain embodiments of Formula (Ia), (Ic), (Ic-1), or (Ic-2),

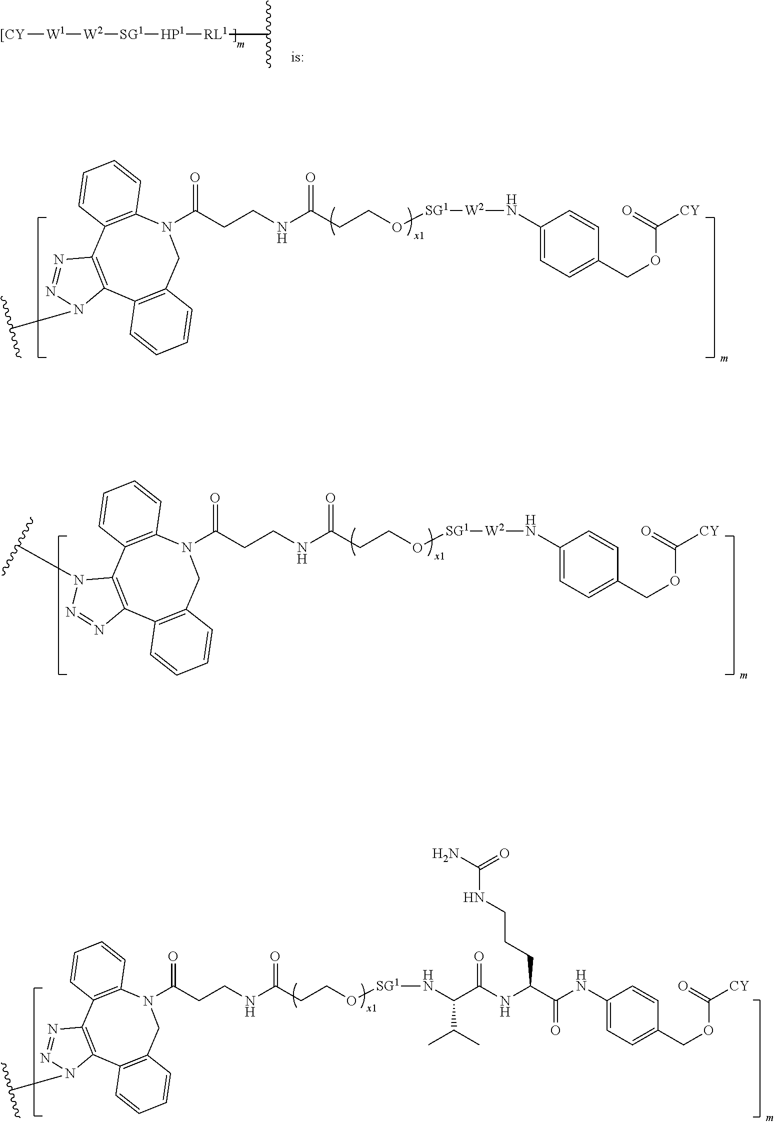

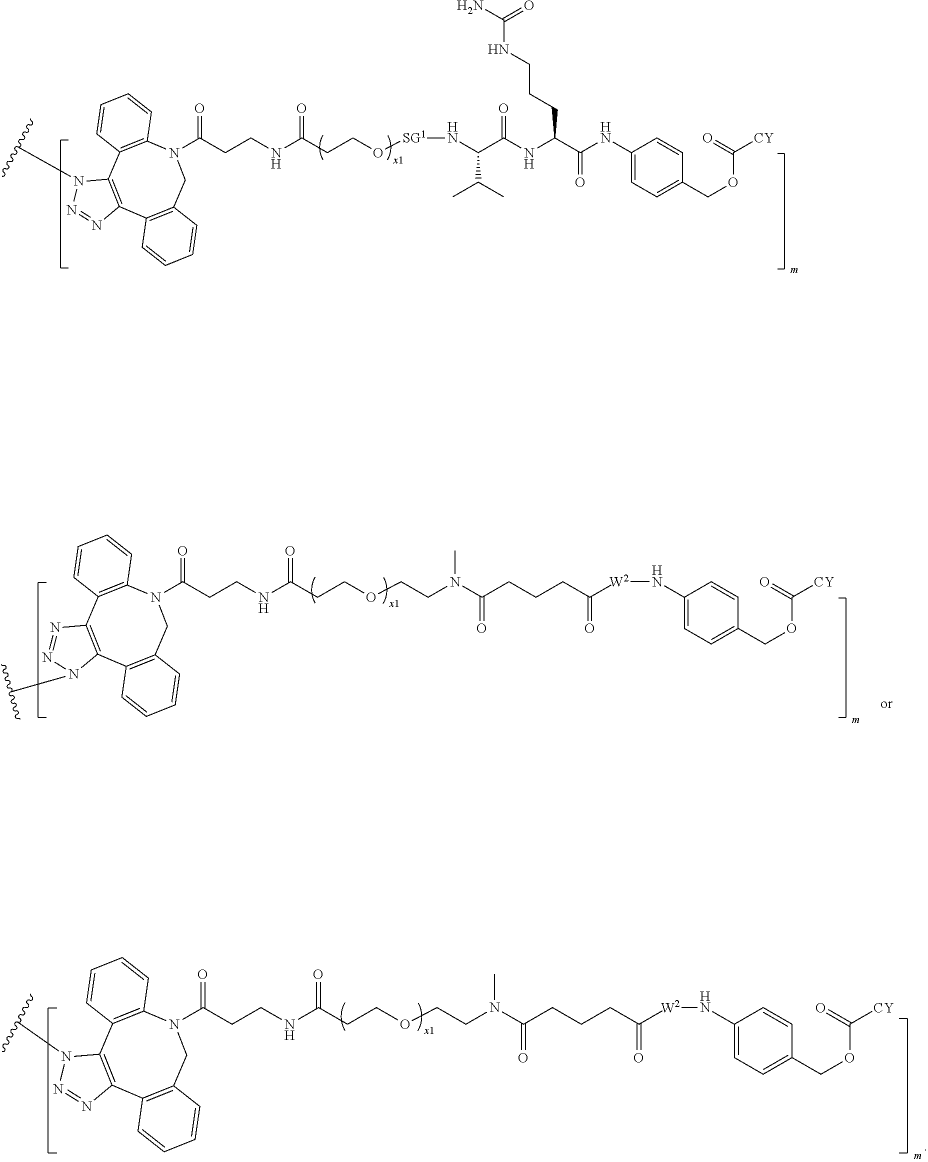

In certain embodiments of Formula (Ia), (Ic), (Ic-1), or (Ic-2),

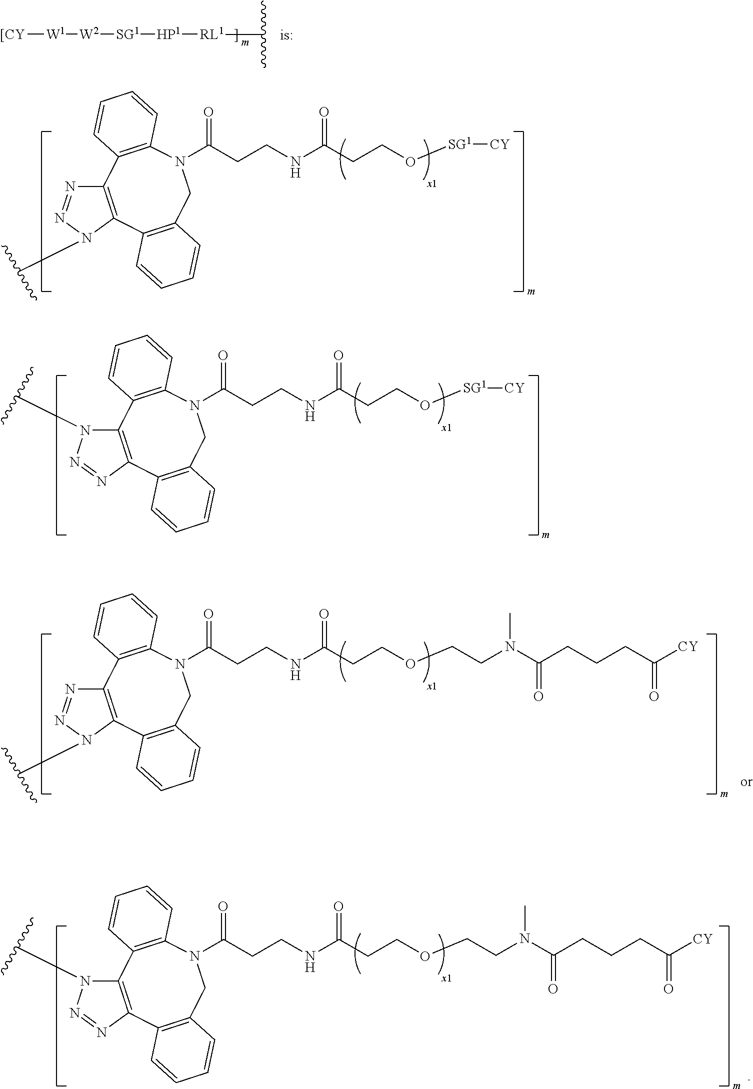

In one embodiment of Formula (I),

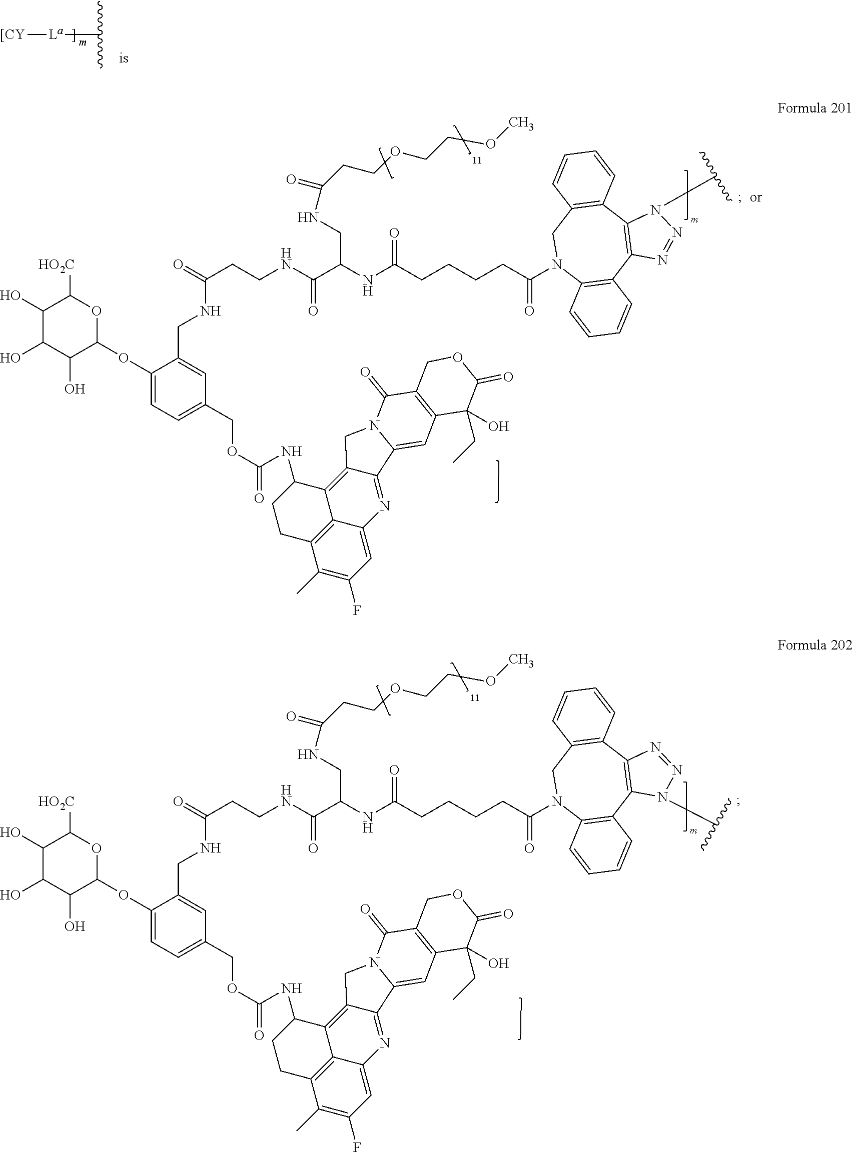

or a mixture of Formula 201 and Formula 202;
wherein [symbol] is the point of attachment to the rest of the formula.

In one embodiment of Formula (I),

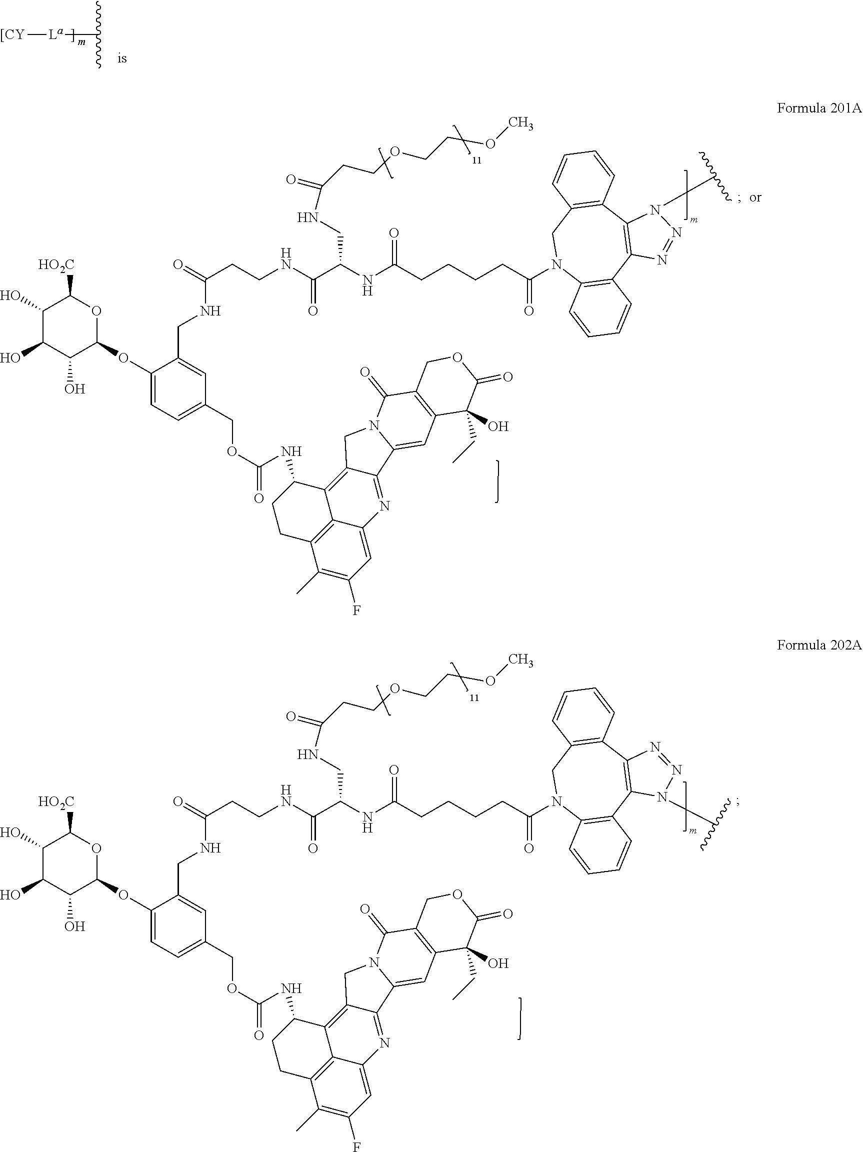

or a mixture of Formula 201A and Formula 202A;
wherein [symbol] is the point of attachment to the rest of the formula.

In one embodiment of Formula (I),

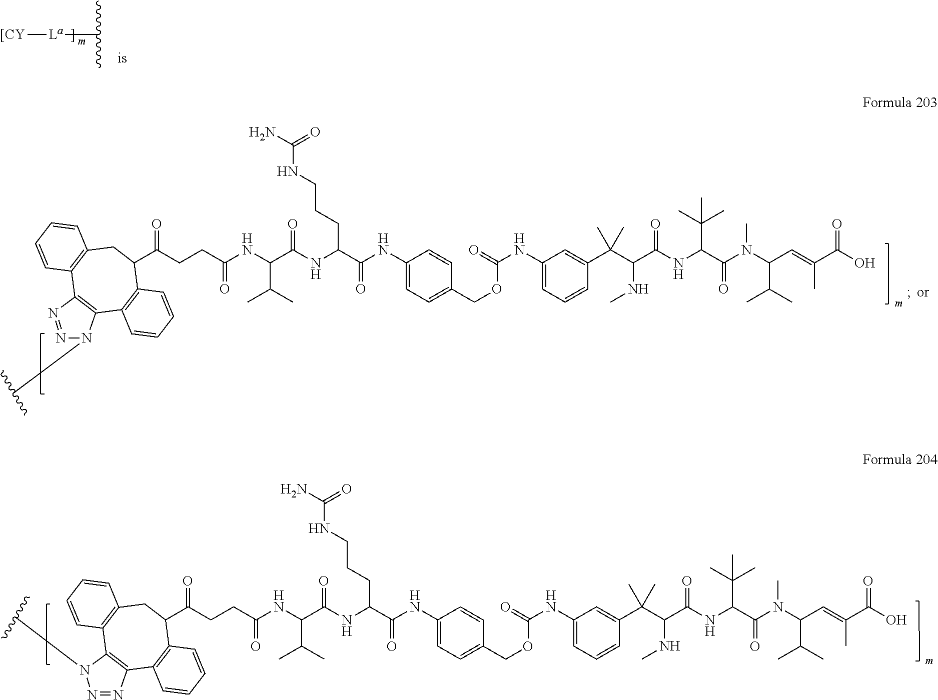

or a mixture of Formula 203 and Formula 204;
wherein [symbol] is the point of attachment to the rest of the formula.

In one embodiment of Formula (I),

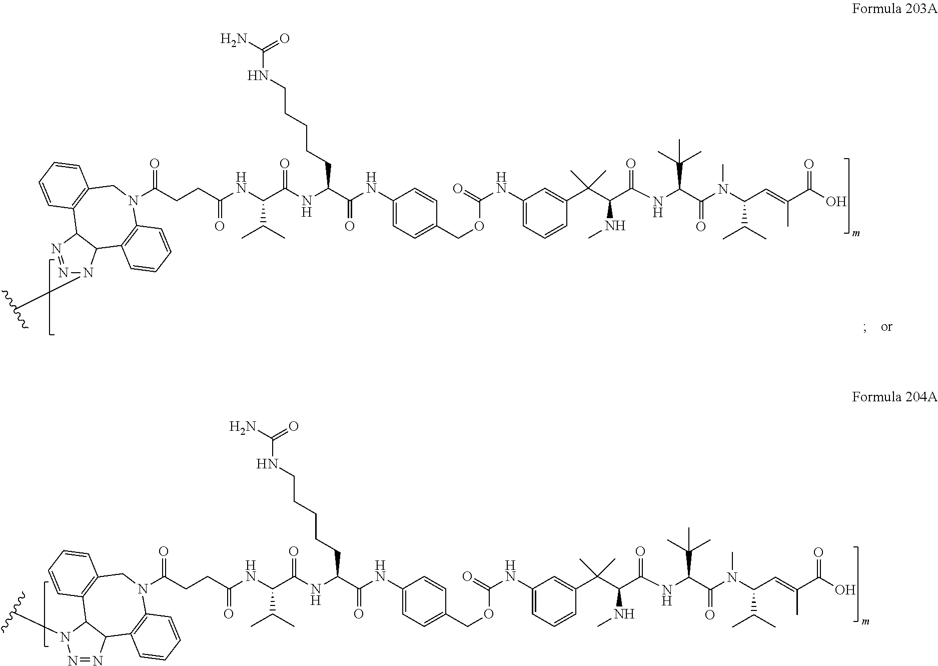

or a mixture of Formula 203A and Formula 204A;
wherein [symbol] is the point of attachment to the rest of the formula.

In one embodiment of Formula (I),

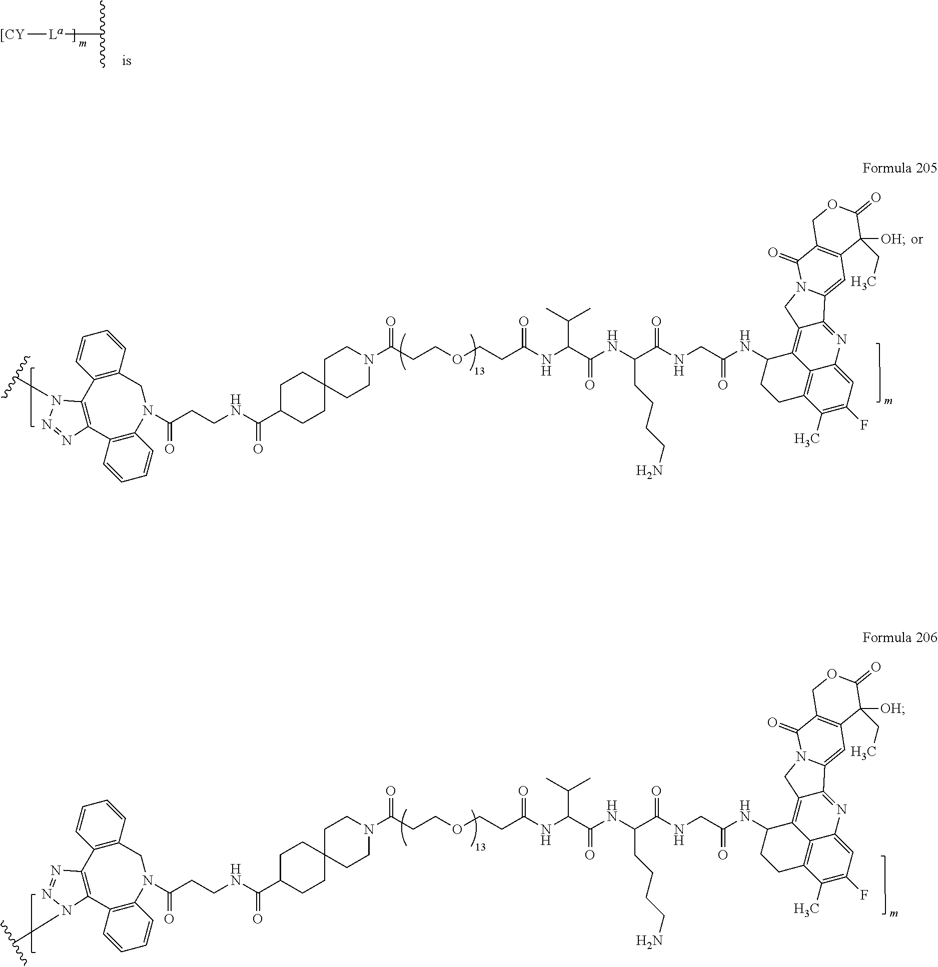

or a mixture of Formula 205 and Formula 206;
wherein [symbol] is the point of attachment to the rest of the formula.

In one embodiment of Formula (I),

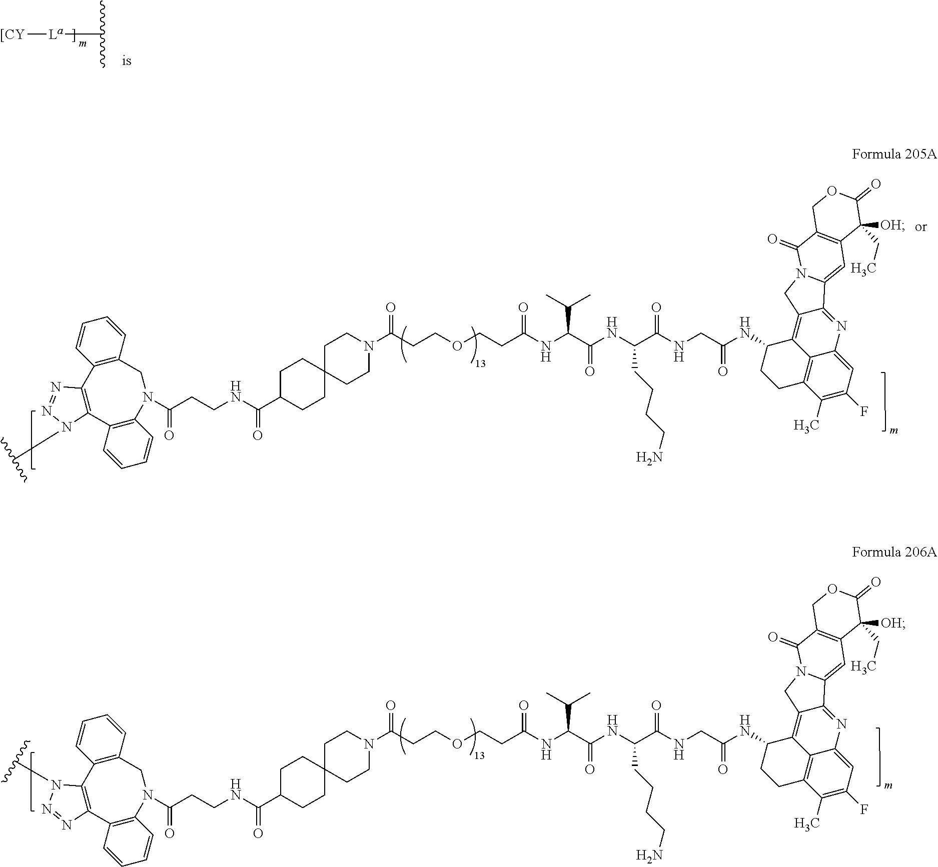

or a mixture of Formula 205A and Formula 206A;
wherein [symbol] is the point of attachment to the rest of the formula.

In one embodiment of Formula (I),

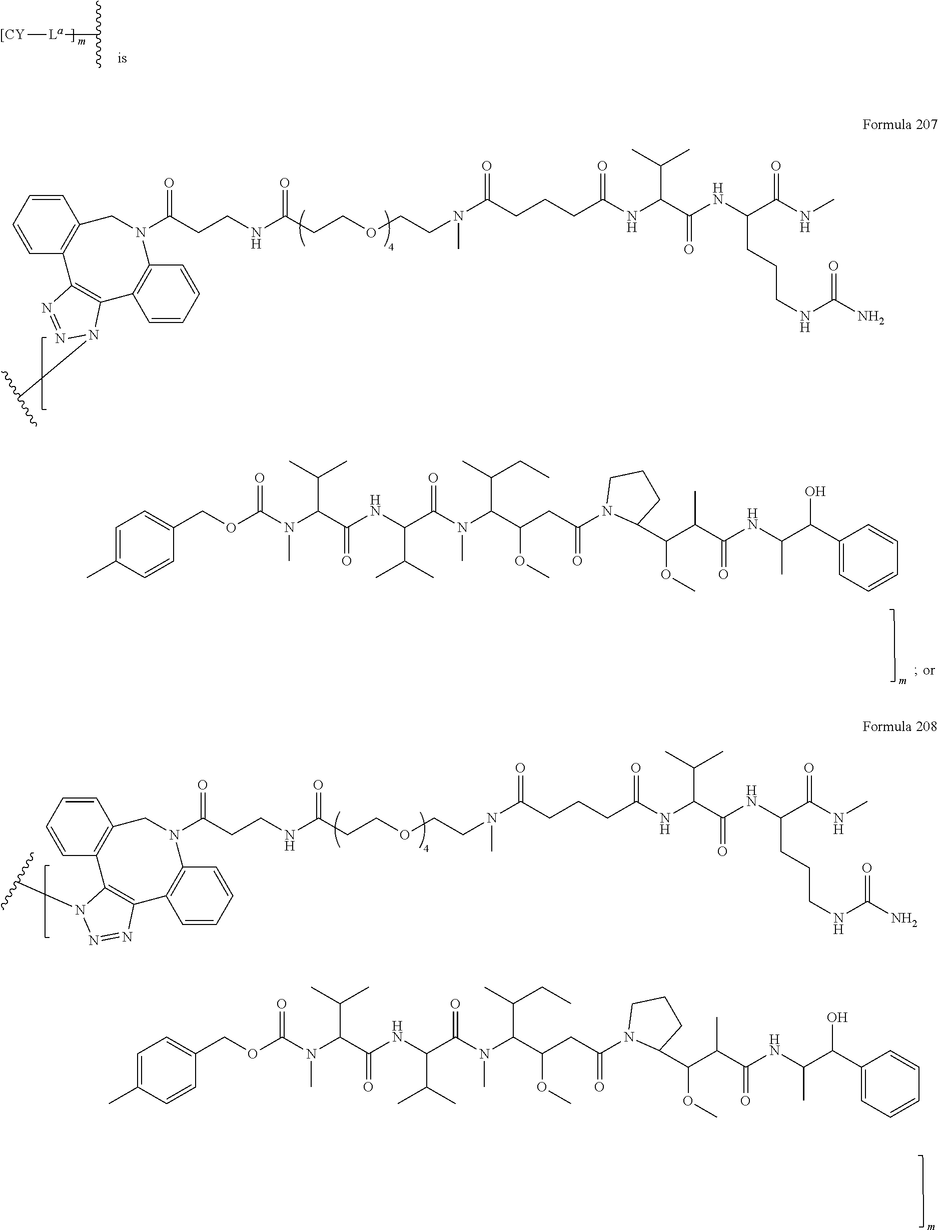

or a mixture of Formula 207 and Formula 208;
wherein [symbol] is the point of attachment to the rest of the formula.

In one embodiment of Formula (I),

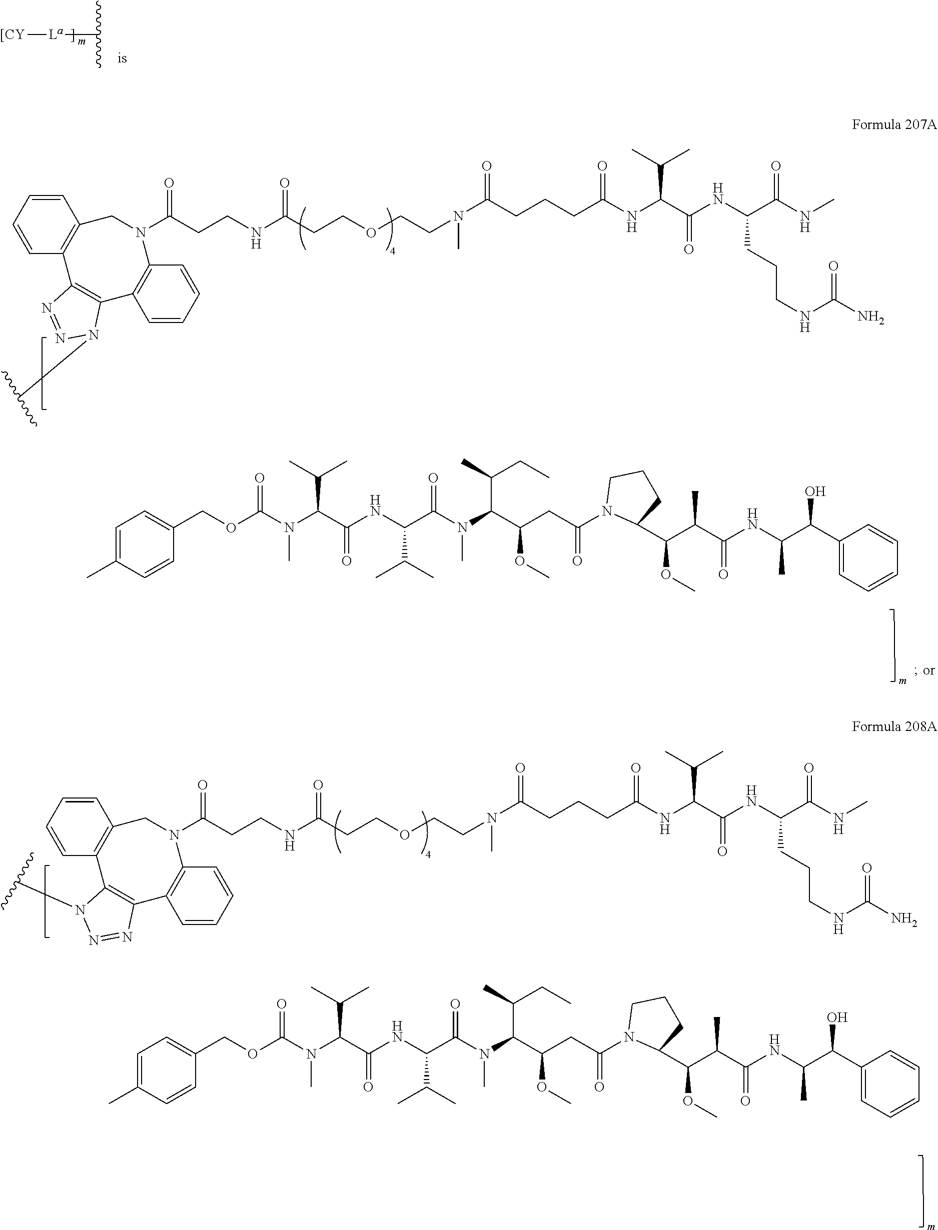

or a mixture of Formula 207A and Formula 208A;
wherein [symbol] is the point of attachment to the rest of the formula.

In one embodiment of Formula (I),

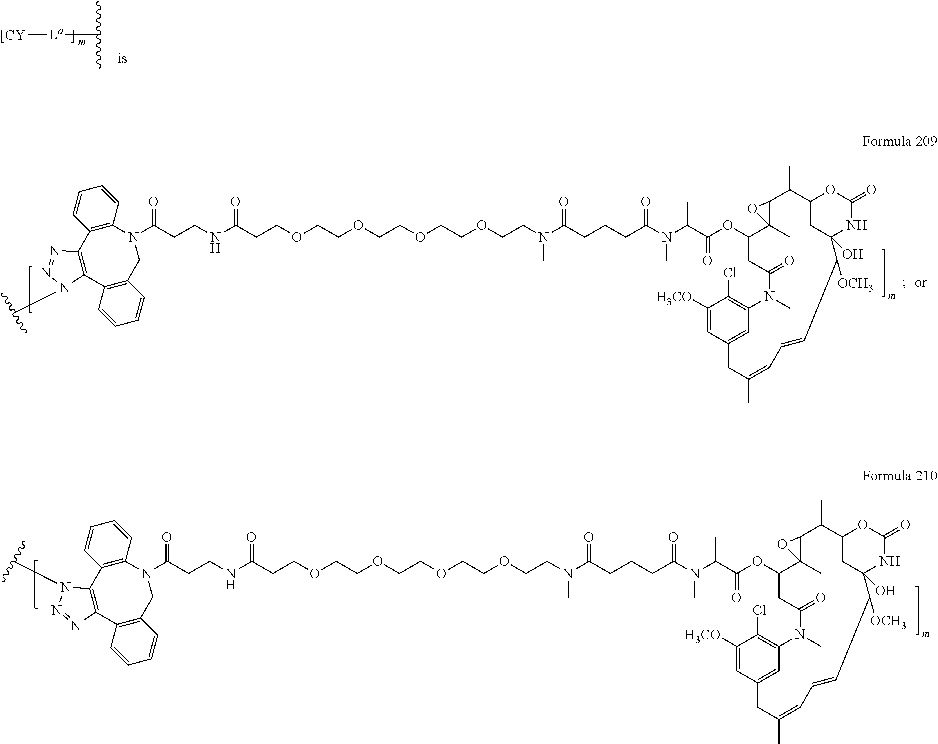

or a mixture of Formula 209 and Formula 210;
wherein [symbol] is the point of attachment to the rest of the formula.

In one embodiment of Formula (I),

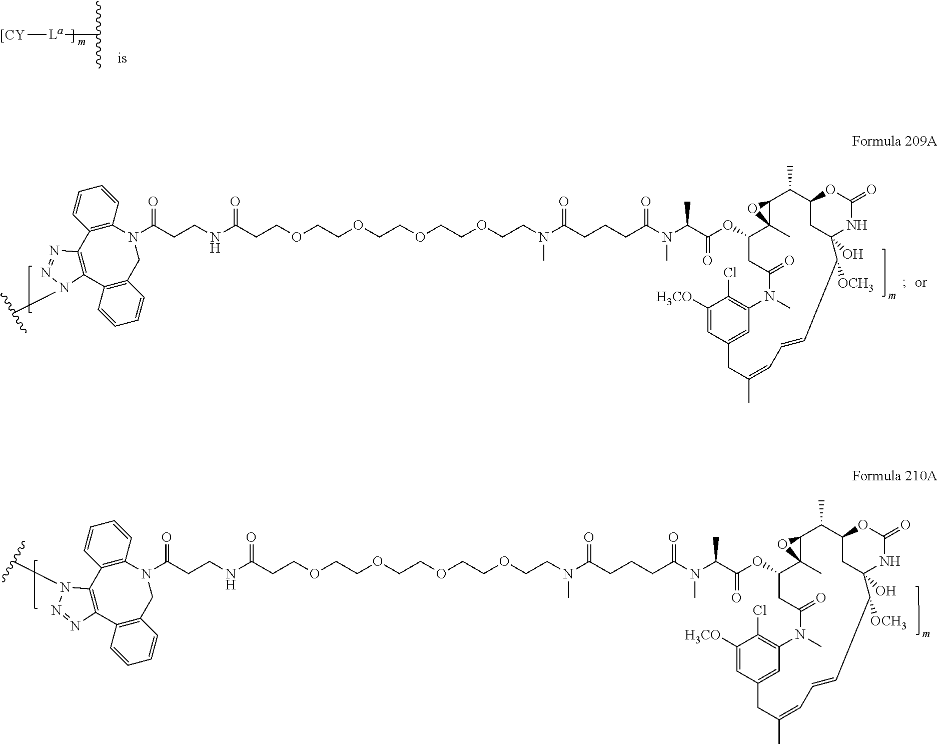

or a mixture of Formula 209A and Formula 210A;
wherein [symbol] is the point of attachment to the rest of the formula.

### STING Agonist (STING)

In some embodiments, the STING agonist is a diamidobenzimidazole (diabzi) STING agonist. In some embodiments of Formula (I), (Ia), (Ib), (Ic), (Ib-1), (Ib-2), (Ic-1), or (Ic-2), including any of the foregoing, the STING agonist is independently a residue of a compound of Formula (S-I):

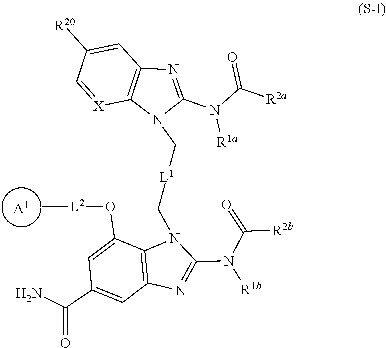

or a tautomer or stereoisomer thereof,
wherein:
X is selected from N and CR³;
R²⁰ is selected from hydrogen and —CON(R^{3a})(R^{3b});
R^{1a}, R^{1b}, R^{3a} and R^{3b} are independently selected from hydrogen and optionally substituted C₁₋₆ alkyl, wherein the C₁₋₆ alkyl is optionally substituted with one or more R⁵⁰;
R^{2a} and R^{2b} are independently selected from: (a) optionally substituted C₁₋₆ alkyl, wherein the C₁₋₆ alkyl is optionally substituted with one or more R⁵¹ and (b) optionally substituted C₃₋₁₂ carbocycle or optionally substituted 3- to 12-membered heterocycle, each of which is optionally substituted with one or more R⁵³;
or R^{1a} and R^{2a} are joined together with the atoms to which they are attached to form an optionally substituted 3- to 12-membered heterocycle, wherein the heterocycle is optionally substituted with one or more R⁵³;
or R^{1b} and R^{2b} are joined together with the atoms to which they are attached to form an optionally substituted 3- to 12-membered heterocycle, wherein the heterocycle is optionally substituted with one or more R⁵³;
R³ is hydrogen, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, or —CN;
L¹ is selected from a bond, —C₁₋₁₀alkylene-, —C₂₋₁₀alkenylene-, —C₂₋₁₀alkynylene-, —C₁₋₆alkylene-O—C₁₋₆alkylene-, —C₁₋₆alkylene-NH—C₁₋₆alkylene-, C₃₋₆carbocyclene, and —C₁₋₆alkylene-(C₃₋₆carbocyclene)-C₁₋₆alkylene-, wherein —C₁₋₁₀ alkylene-, —C₂₋₁₀ alkenylene-, —C₂₋₁₀ alkynylene-, C₃₋₆ carbocyclene, and each C₁₋₆alkylene of —C₁₋₆alkylene-O—C₁₋₆alkylene-, —C₁₋₆alkylene-NH—C₁₋₆alkylene-, and —C₁₋₆alkylene-(C₃₋₆carbocyclene)-C₁₋₆alkylene- are optionally substituted with one or more R⁵⁰;
L² is optionally substituted —C₁₋₆ alkylene- or —C₁₋₆alkylene-O—, wherein the —C₁₋₆ alkylene- or the C₁₋₆ alkylene of —C₁₋₆ alkylene-O— is optionally substituted with one or more R⁵⁰ and wherein the —C₁₋₆ alkylene-O— is attached to the A ring, or L^{b} if the A ring is absent, via the oxygen atom;
Ring A is absent; an optionally substituted bridged, fused, or spirocyclic bicyclic heterocycle comprising at least one N atom and at least one O atom, wherein the heterocycle is optionally substituted with one or more R⁵³; or, a 3- to 12-membered heterocycle substituted with R⁴;
R⁴ is an optionally substituted 3- to 12-membered heterocycle optionally comprising at least one NR⁵ and, wherein the heterocycle is optionally substituted with one or more R⁵³;
R⁵ is independently selected from hydrogen, R⁶, —C(O)—C₁₋₆alkyl, —C(O)-heteroC₁₋₆alkyl, C₁₋₆ alkyl, and heteroC₁₋₆alkyl wherein the C₁₋₆ alkyl, either alone or part of another group, is optionally substituted with one or more R⁵⁰;
R⁶ is independently an amino acid residue;
R³⁰ is independently selected from hydrogen, C₁₋₁₀ alkyl, C₂₋₁₀ alkenyl, C₂₋₁₀ alkynyl, C₃₋₁₂ carbocycle, and 3- to 12-membered heterocycle, each of which, except hydrogen, is optionally substituted with one or more R⁵⁵;
R⁵⁰ is independently selected from halogen, —OR⁶⁰, —SR⁶⁰, —C(O)NR⁶⁰R⁶¹, —N(R⁶⁰)C(O)R⁶⁰, —N(R⁶⁰)C(O)NR⁶⁰R⁶¹, —NR⁶⁰R⁶¹, —C(O)R⁶⁰, —C(O)OR⁶⁰, —OC(O)R⁶⁰, —NO₂, ═O, ═S, ═N(R⁶⁰), —CN, C₃₋₁₂ carbocycle, and 3- to 12-membered heterocycle;
R⁵¹ is independently selected from halogen, —OR⁶⁰, —SR⁶⁰, —C(O)NR⁶⁰R⁶¹, —N(R⁶⁰)C(O)R⁶⁰, —N(R⁶⁰)C(O)NR⁶⁰R⁶¹, —NR⁶⁰R⁶¹, —C(O)R⁶⁰, —C(O)OR⁶⁰, —OC(O)R⁶⁰, —NO₂, ═O, ═S, ═N(R⁶⁰), —CN, optionally substituted C₃₋₁₂ carbocycle, and optionally substituted 3- to 12-membered heterocycle, wherein the C₃₋₁₂ carbocycle and 3- to 12-membered heterocycle are optionally substituted with one or more R⁵²;
R⁵² is independently selected from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), —CN, C₁₋₆ alkyl, C₂₋₆ alkenyl, and C₂₋₆ alkynyl;
R⁵³ is independently selected from halogen, —OR⁶⁰, —SR⁶⁰, —C(O)NR⁶⁰R⁶¹, —N(R⁶⁰)C(O)R⁶⁰, —N(R⁶⁰)C(O)NR⁶⁰R⁶¹, —NR⁶⁰R⁶¹, —C(O)R⁶⁰, —C(O)OR⁶⁰, —OC(O)R⁶⁰, —NO₂, ═O, ═S, ═N(R⁶⁰), —CN, optionally substituted C₁₋₆ alkyl, optionally substituted C₂₋₆ alkenyl, optionally substituted C₂₋₆ alkynyl, optionally substituted C₃₋₁₂ carbocycle, and optionally substituted 3- to 12-membered heterocycle, wherein the C₁₋₆ alkyl, C₂₋₆ alkenyl, and C₂₋₆ alkynyl are optionally substituted with one or more R⁵⁴ and the C₃₋₁₂ carbocycle and 3- to 12-membered heterocycle are optionally substituted with one or more R⁵²;
R⁵⁴ is independently selected from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;
R⁵⁵ is independently selected from halogen, —CN, —NO₂, —OH, —NR⁶⁰R⁶¹, —C(O)NR⁶⁰R⁶¹, ═O, ═S, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl; and
R⁶⁰ and R⁶¹ are independently selected from hydrogen, C₁₋₁₀ alkyl optionally substituted with NH₂ or NHC₁₋₆ alkyl, C₂₋₁₀ alkenyl, C₂₋₁₀ alkynyl, C₃₋₁₂ carbocycle, and 3- to 12-membered heterocycle;
wherein when Ring A is absent, L² is —C₁₋₆ alkylene-O—;
wherein the residue of Formula (S-I) is attached to L^{b} via an amino group of Ring A, the oxygen of —C₁₋₆ alkylene-O— of L², an amino group of R⁴, an amino group of R⁵⁴, or an amino group of R^{2b} when R^{2b} is an optionally substituted C₃₋₁₂ carbocycle or optionally substituted 3- to 12-membered heterocycle.

In some embodiments, the STING agonist is independently a residue of a compound of Formula (S-I) wherein R^{1a} and R^{1b} are both hydrogen.

In some embodiments, the STING agonist is independently a residue of a compound of Formula (S-I) wherein R^{2a} and R^{2b} are independently an optionally substituted 3- to 12-membered heterocycle comprising at least one N atom.

In some embodiments, the STING agonist is independently a residue of a compound of Formula (S-I) wherein R^{2a} and R^{2b} are independently selected from

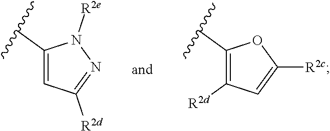

wherein R^{2c} and R^{2d} are independently selected from hydrogen, C₁₋₆ alkyl optionally substituted with one or more R⁵⁴, halo, and 3-12 membered heterocycle;
R^{2e} is hydrogen or C₁₋₆ alkyl; and
[symbol] is the point of attachment to the rest of the formula.

In some embodiments, the STING agonist is independently a residue of a compound of Formula (S-I)=wherein Ring A is an optionally substituted 3- to 12-membered N-linked spirocyclic bicyclic heterocycle comprising at least one nitrogen atom, including the nitrogen bound to L^{b}, and at least one oxygen atom; or a 3- to 12-membered N-linked monocyclic heterocycle substituted with R⁴.

In some embodiments, the STING agonist is independently a residue of a compound of Formula (S-I) wherein

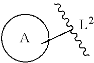

is selected from

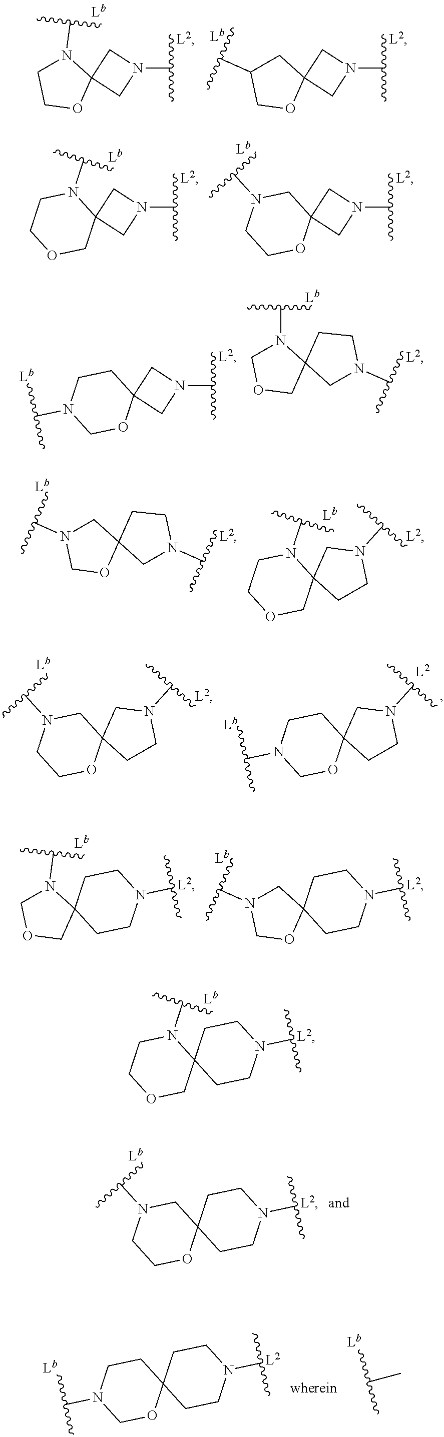

is the point of attachment to L^{b} and [symbol] is the point of attachment to L².

In some embodiments, the STING agonist is independently a residue of a compound of Formula (S-I) wherein

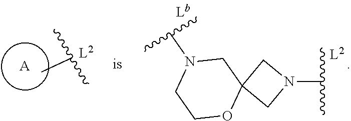

In some embodiments, the STING agonist is independently a residue of a compound of Formula (S-I) wherein Ring A is an N-linked mono 3- to 12-membered heterocycle substituted with R⁴.

In some embodiments, the STING agonist is independently a residue of a compound of Formula (S-I) wherein Ring A is selected from

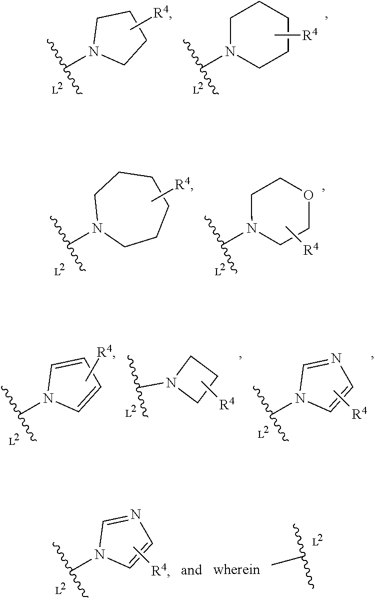

is the point of attachment to L².

In some embodiments, the STING agonist is independently a residue of a compound of Formula (S-I) wherein Ring A is

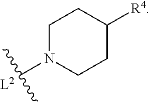

In some embodiments, the STING agonist is independently a residue of a compound of Formula (S-I) wherein R⁴ is a 5-5 fused ring system, a 5-6 fused ring system, a 6-6 fused ring system, or a 5-7 fused ring system comprising 1, 2, 3, or 4 heteroatoms selected from N, S, and O wherein at least one heteroatom is N bound to L^{b}.

In some embodiments, the STING agonist is independently a residue of a compound of Formula (S-I) wherein R⁴ is

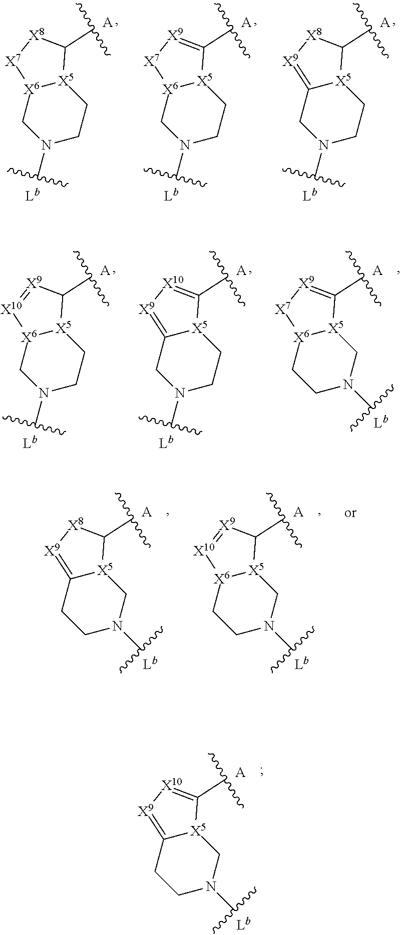

wherein X⁵, X⁶, X⁹, X¹⁰ are independently selected from N and CR¹³;
X⁷ and X⁸ are independently selected from NH, O, and CHR¹³;
R¹³ is independently hydrogen or C₁₋₆ alkyl;
wherein if four of any one of X⁵—X¹⁰ are present, at least one is CR¹³ or CHR¹³; and
[symbol] is the point of attachment to ring A and [symbol] is the point of attachment to L^{b}.

In some embodiments, the STING agonist is independently a residue of a compound of Formula (S-I) wherein R⁴ is

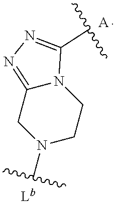

In some embodiments of Formula (I), (Ia), (Ib), (Ic), (Ib-1), (Ib-2), (Ic-1), or (Ic-2), including any of the foregoing, the STING agonist is independently a residue of a compound of Formula (S-II):

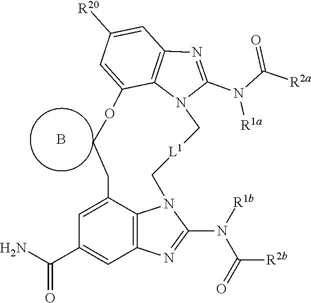

or a tautomer or stereoisomer thereof,
wherein:
Ring B is an optionally substituted C₃₋₁₂ carbocycle or an optionally substituted 3- to 12-membered heterocycle, wherein the C₃₋₁₂ carbocycle and 3- to 12-membered heterocycle are optionally substituted with one or more R⁵²;
R^{1a}, R^{2a}, R^{1b}, R^{2b}, L¹, R²⁰, and R⁵² are as defined herein; and
wherein the residue of Formula (S-II) is attached to L^{b} via an amino group of Ring B.

In some embodiments, the STING agonist is independently a residue of a compound of Formula (S-II) wherein ring B is an optionally substituted azetidine.

In some embodiments of Formula (I), (Ia), (Ib), (Ic), (Ib-1), (Ib-2), (Ic-1), or (Ic-2), including any of the foregoing, the STING agonist is independently a residue of a compound of Formula (S-III):

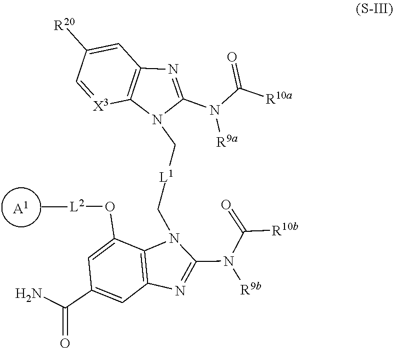

or a pharmaceutically acceptable salt thereof or tautomer thereof,
wherein:
Ring A¹ is C₃₋₁₂ carbocycle or 3- to 12-membered heterocycle, each of which is optionally substituted with one or more R⁵³;
X³ is selected from N and CR³;
R³ is selected from hydrogen, —OR³⁰, —SR³⁰, —C(O)N(R³⁰)₂, —N(R³⁰)C(O)R³⁰, —N(R³⁰)C(O)N(R³⁰)₂, —N(R³⁰)₂, —C(O)R³⁰, —C(O)OR³⁰, —OC(O)R³⁰, —NO₂, and —CN;
each R³⁰ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more R⁵⁵;
R^{9a}, R^{10a}, R^{9b}, R^{10b}, L¹, L², R²⁰, R³⁰, R⁵³, and R⁵⁵ are as defined herein; and
wherein the residue of Formula (S-III) is attached to L^{b} via an amino group of Ring A¹.

In some embodiments of Formula (I), (Ia), (Ib), (Ic), (Ib-1), (Ib-2), (Ic-1), or (Ic-2), including any of the foregoing, the STING agonist is independently a residue of a compound of Formula (S-IV):

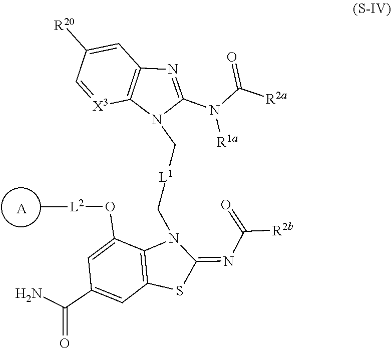

or a pharmaceutically acceptable salt thereof or tautomer thereof,
wherein Ring A, L¹, L², X³, R^{1a}, R^{2a}, R^{2b}, and R²⁰ are as defined herein; and
wherein the residue of Formula (S-IV) is attached to L^{b} via an amino group of Ring A, the oxygen of —C₁₋₆ alkylene-O— of L², or an amino group of R⁴.

### Embodiments of Formulas (S-I)-(S-IV) when Used as STING in Conjugates of Formula (I), (Ia), (Ib), (Ic), (Ib-1), (Ib-2), (Ic-1), or (Ic-2)

In one embodiment of Formula (S-I), (S-II), or (S-IV), R^{1a} is hydrogen. In one embodiment of Formula (S-I) or (S-II), R^{1b} is hydrogen. In one embodiment of Formula (S-I) or (S-II), R^{1a} is C₁₋₆ alkyl, for example methyl. In one embodiment of Formula (S-I) or (S-II), R^{1b} is C₁₋₆ alkyl, for example methyl. In one embodiment of Formula (S-I) or (S-II), R^{1a} and R^{1b} are both hydrogen.

In one embodiment of Formula (S-I), (S-II), or (S-IV), including any of the foregoing, R^{2a} is a 3- to 12-membered heterocycle. In one embodiment of Formula (S-I), (S-II), or (S-IV), including any of the foregoing, R^{2a} is a 5- to 6-membered heterocycle. In one embodiment of Formula (S-I), (S-II), or (S-IV), including any of the foregoing, R^{2a} is a 3- to 12-membered heterocycle containing at least one N atom. In one embodiment of Formula (S-I), (S-II), or (S-IV), including any of the foregoing, R^{2a} is a 5- to 6-membered heterocycle containing at least one N atom. In one embodiment of Formula (S-I), (S-II), or (S-IV), including any of the foregoing, R^{2a} is a 3- to 12-membered heterocycle containing at least one N atom and the heterocycle is further substituted with at least one R⁵³. In one embodiment of Formula (S-I), (S-II), or (S-IV), including any of the foregoing, R^{2a} is a 5- to 6-membered heterocycle containing at least one N atom and the heterocycle is further substituted with at least one R⁵³. In one embodiment of Formula (S-I), (S-II), or (S-IV), including any of the foregoing, R^{2a} is a 3- to 12-membered heterocycle containing at least one N atom and the heterocycle is further substituted with at least one R⁵³ selected from C₁₋₆ alkyl, halogen, and haloC₁₋₆ alkyl. In one embodiment of Formula (S-I), (S-II), or (S-IV), including any of the foregoing, R^{2a} is a 5- to 6-membered heterocycle containing at least one N atom and the heterocycle is further substituted with at least one R⁵³ selected from C₁₋₆ alkyl, halogen, and haloC₁₋₆ alkyl. In one embodiment of Formula (S-I), (S-II), or (S-IV), including any of the foregoing, R^{2b} is a 3- to 12-membered heterocycle containing at least one N atom. In one embodiment of Formula (S-I), (S-II), or (S-IV), including any of the foregoing, R^{2b} is a 5- to 6-membered heterocycle containing at least one N atom. In one embodiment of Formula (S-I), (S-II), or (S-IV), including any of the foregoing, R^{2b} is a 3- to 12-membered heterocycle containing at least one N atom and the heterocycle is further substituted with at least one R⁵³ In one embodiment of Formula (S-I), (S-II), or (S-IV), including any of the foregoing, R^{2b} is a 5- to 6-membered heterocycle containing at least one N atom and the heterocycle is further substituted with at least one R⁵³. In one embodiment of Formula (S-I), (S-II), or (S-IV), including any of the foregoing, R^{2b} is a 3- to 12-membered heterocycle containing at least one N atom and the heterocycle is further substituted with at least one R⁵³ selected from C₁₋₆ alkyl, halogen, and haloC₁₋₆ alkyl. In one embodiment of Formula (S-I), (S-II), or (S-IV), including any of the foregoing, R^{2b} is a 5- to 6-membered heterocycle containing at least one N atom and the heterocycle is further substituted with at least one R⁵³ selected from C₁₋₆ alkyl, halogen, and haloC₁₋₆ alkyl. In one embodiment of Formula (S-I), (S-II), or (S-IV), including any of the foregoing, R^{2a} and R^{2b} are both a 3- to 12-membered heterocycle containing at least one N atom. In one embodiment of Formula (S-I), (S-II), or (S-IV), including any of the foregoing, R^{2a} and R^{2b} are both a 5- to 6-membered heterocycle containing at least one N atom. In one embodiment Formula (S-I), (S-II), or (S-IV), including any of the foregoing, R^{2a} and R^{2b} are both a 3- to 12-membered heterocycle containing at least one N atom and the heterocycle is further substituted with at least one R⁵³ selected from C₁₋₆ alkyl, halogen, and haloC₁₋₆ alkyl. In one embodiment of Formula (S-I), (S-II), or (S-IV), including any of the foregoing, R^{2a} and R^{2b} are both a 5- to 6-membered heterocycle containing at least one N atom and the heterocycle is further substituted with at least one R⁵³ selected from C₁₋₆ alkyl, halogen, and haloC₁₋₆ alkyl.

In one embodiment of Formula (S-I) or (S-II), including any of the foregoing, R^{1a} is hydrogen, R^{1b} is hydrogen, and R^{2a} and R^{2b} are both a 3- to 12-membered heterocycle containing at least one N atom and the heterocycle is further substituted with at least one R⁵³ selected from C₁₋₆ alkyl, halogen, and haloC₁₋₆ alkyl.

In one embodiment of Formula (S-I), (S-II), or (S-IV), including any of the foregoing, R^{2a} and/or R^{2b} are independently selected from an optionally substituted pyrazole, an optionally substituted oxazole, an optionally substituted thiazole, an optionally substituted pyrrolidine, an optionally substituted phenyl, an optionally substituted pyridine, and an optionally substituted pyridazine. In one embodiment of Formula (S-I), (S-II), or (S-IV), including any of the foregoing, R^{2a} and/or R^{2b} are independently selected from an optionally substituted pyrazole, an optionally substituted oxazole, an optionally substituted thiazole, an optionally substituted pyrrolidine, and an optionally substituted pyridazine. In one embodiment of Formula (S-I), (S-II), or (S-IV), including any of the foregoing, R^{2a} and/or R^{2b} are independently selected from an optionally substituted pyrazole, an optionally substituted oxazole, and an optionally substituted thiazole.

In one embodiment of Formula (S-I), (S-II), or (S-IV), including any of the foregoing, R^{2a} and/or R^{2b} are independently selected from

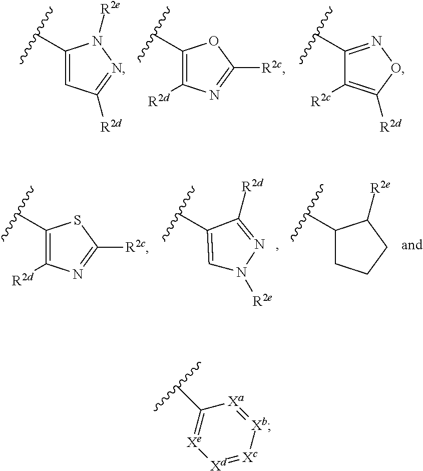

wherein R^{2c} and R^{2d} are independently hydrogen, halo, C₁₋₆ alkyl optionally substituted with one or more R⁵⁴, or 3- to 12-membered heterocycle;
R^{2e} is hydrogen or C₁₋₆ alkyl;
X^{a}, X^{b}, X^{c}, X^{d}, and X^{e} are independently selected from —N— and —CR^{2c}— wherein no more than two of X^{a}-X^{e} are N;
R⁵⁴ is independently selected from halogen, —OR⁶¹, —SR⁶¹, —C(O)N(R⁶¹)₂, —N(R⁶¹)C(O)R⁶¹, —N(R⁶¹)C(O)N(R⁶¹)₂, —N(R⁶¹)₂, —C(O)R⁶¹, —C(O)OR⁶¹, —OC(O)R⁶¹, —NO₂, ═O, ═S, ═N(R⁶¹), and —CN;
R⁶¹ is independently selected at each occurrence from hydrogen, C₁₋₁₀ alkyl, C₂₋₁₀ alkenyl, C₂₋₁₀ alkynyl, C₃₋₁₂ carbocycle, and 3- to 12-membered heterocycle; and
[symbol] is the point of attachment to the rest of the compound.

In one embodiment of Formula (S-I), (S-II), or (S-IV), including any of the foregoing, R^{2e} and R^{2d} are both C₁₋₆ alkyl. In one embodiment of Formula (S-I), (S-II), or (S-IV), including any of the foregoing, R^{2e} is C₁₋₆ alkyl and R^{2d} is halo. In one embodiment of Formula (S-I), (S-II), or (S-IV), including any of the foregoing, R^{2e} is C₁₋₆ alkyl and R^{2d} is C₁₋₆ alkyl optionally substituted with one or more R⁵⁴. In one embodiment of Formula (S-I), (S-II), or (S-IV), including any of the foregoing, R^{2e} is C₁₋₆ alkyl and R^{2d} is haloC₁₋₆ alkyl. In one embodiment of Formula (S-I), (S-II), or (S-IV), including any of the foregoing, R^{2e} is C₁₋₆ alkyl and R^{2d} is aminoC₁₋₆ alkyl. In one embodiment of Formula (S-I), (S-II), or (S-IV), including any of the foregoing, R^{2e} is C₁₋₆ alkyl and R^{2d} is 3- to 12-membered heterocycle. In one embodiment of Formula (S-I), (S-II), or (S-IV), including any of the foregoing, R^{2e} is C₁₋₆ alkyl and R^{2d} is hydrogen.

In one embodiment of Formula (S-I), (S-II), or (S-IV), including any of the foregoing, R^{2c} and R^{2d} are both C₁₋₆ alkyl. In one embodiment of Formula (S-I), (S-II), or (S-IV), including any of the foregoing, R^{2c} is hydrogen and R^{2d} is C₁₋₆ alkyl.

In one embodiment of Formula (S-I), (S-II), or (S-IV), including any of the foregoing,

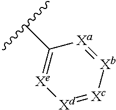

is selected from

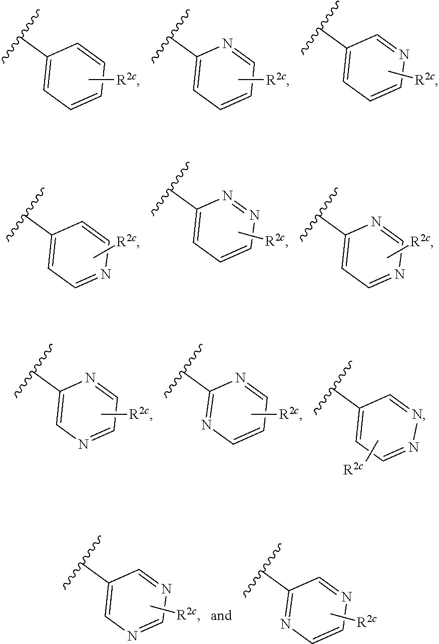

and R^{2c} is C₁₋₆ alkyl.

In one embodiment of Formula (S-I), (S-II), or (S-IV), including any of the foregoing,

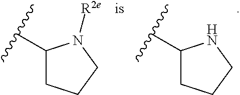

In one embodiment of Formula (S-I), (S-II), or (S-IV), including any of the foregoing, R^{2a} and/or R^{2b} are independently selected from

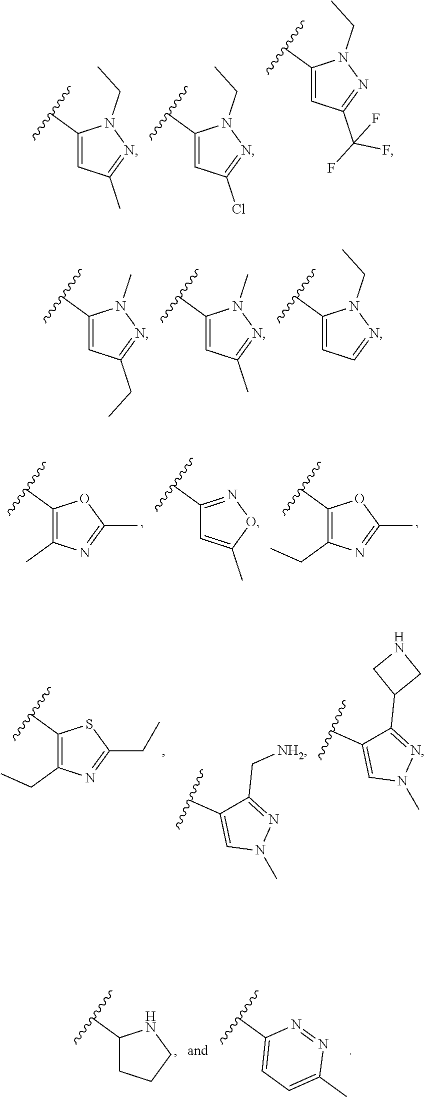

In one embodiment of Formula (S-I), (S-II), or (S-IV), including any of the foregoing, R^{2a} and/or R^{2b} are independently selected from

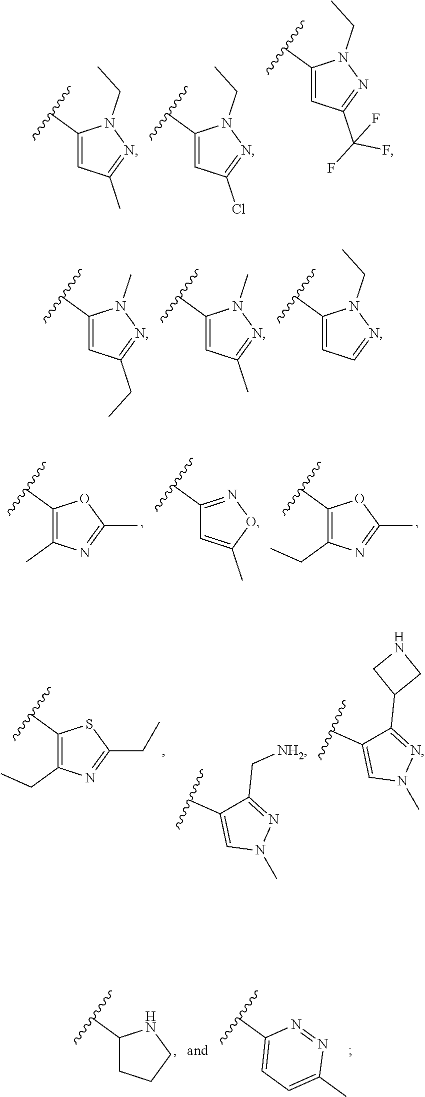

and R^{1a} and R^{1b} are both hydrogen.

In one embodiment of Formula (S-I), (S-II), or (S-IV), including any of the foregoing, R^{2a} and R^{2b} are both

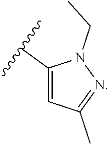 [SMILES: *c1cc(C)nn1CC]

In one embodiment of Formula (S-I), (S-II), or (S-IV), including any of the foregoing, R^{2a} and R^{2b} are both

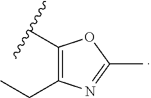 [SMILES: *c1oc(C)nc1CC ; "."]

In one embodiment of Formula (S-I), (S-II), or (S-IV), including any of the foregoing, R^{2a} is

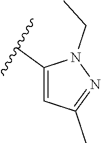 [SMILES: *c1cc(C)nn1CC]

### and R^{2b} is

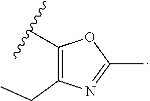 [SMILES: *c1oc(C)nc1CC ; "."]

In one embodiment of Formula (S-I), (S-II), or (S-IV), including any of the foregoing, R^{2b} is

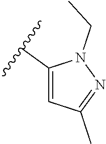 [SMILES: *c1cc(C)nn1CC]

### and R^{2a} is

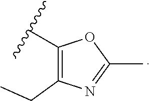 [SMILES: *c1oc(C)nc1CC ; "."]

In one embodiment of Formula (S-I) or (S-II), including any of the foregoing, R^{2a} and R^{2b} are both

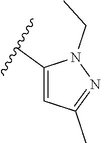 [SMILES: *c1cc(C)nn1CC]

and R^{1a} and R^{1b} are both hydrogen. In one embodiment of Formula (S-I) or (S-II), including any of the foregoing, R^{2a} is

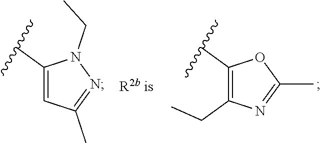

and R^{1a} and R^{1b} are both hydrogen. In one embodiment of Formula (S-I) or (S-II), including any of the foregoing, R^{2a} is

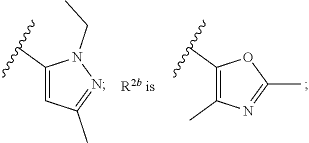

and R^{1a} and R^{1b} are both hydrogen. In one embodiment of Formula (S-I) or (S-II), including any of the foregoing, R^{2b} is

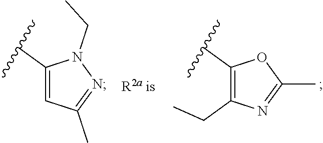

and R^{1a} and R^{1b} are both hydrogen.

In one embodiment of Formula (S-I), (S-II), or (S-IV), including any of the foregoing, R^{2a} is optionally substituted C₁₋₆ alkyl, wherein the C₁₋₆ alkyl is optionally substituted with one or more R⁵¹. In one embodiment of Formula (S-I), (S-II), or (S-IV), including any of the foregoing, R^{2a} is optionally substituted C₁₋₆ alkyl, wherein the C₁₋₆ alkyl is optionally substituted with one R⁵¹ and R⁵¹ is 3- to 12-membered heterocycle optionally substituted with one or more R⁵². In one embodiment of Formula (S-I), (S-II), or (S-IV), including any of the foregoing, R^{2a} is optionally substituted C₁₋₆ alkyl, wherein the C₁₋₆ alkyl is optionally substituted with one R⁵¹ and R⁵¹ is 5- to 6-membered heterocycle optionally substituted with one or more R⁵². In one embodiment of Formula (S-I), (S-II), or (S-IV), including any of the foregoing, R^{2a} is —CH₂—R⁵¹ wherein R⁵¹ is 3- to 12-membered heterocycle optionally substituted with one or more R⁵² and R⁵² is C₁₋₆ alkyl. In one embodiment of Formula (S-I), (S-II), or (S-IV), including any of the foregoing, R^{2a} is —CH₂—R⁵¹ wherein R⁵¹ is 5- to 6-membered heterocycle optionally substituted with one or more R⁵² and R⁵² is C₁₋₆ alkyl. In one embodiment of Formula (S-I), (S-II), or (S-IV), including any of the foregoing, R^{2a} is —CH₂—R⁵¹ wherein R⁵¹ is an optionally substituted pyrazole, an optionally substituted oxazole, or an optionally substituted thiazole.

In one embodiment of Formula (S-I), (S-II), or (S-IV), including any of the foregoing, R^{2b} is optionally substituted C₁₋₆ alkyl, wherein the C₁₋₆ alkyl is optionally substituted with one or more R⁵¹. In one embodiment of Formula (S-I), (S-II), or (S-IV), including any of the foregoing, R^{2b} is optionally substituted C₁₋₆ alkyl, wherein the C₁₋₆ alkyl is optionally substituted with one R⁵¹ and R⁵¹ is 3- to 12-membered heterocycle optionally substituted with one or more R⁵². In one embodiment of Formula (S-I), (S-II), or (S-IV), including any of the foregoing, R^{2b} is optionally substituted C₁₋₆ alkyl, wherein the C₁₋₆ alkyl is optionally substituted with one R⁵¹ and R⁵¹ is 5- to 6-membered heterocycle optionally substituted with one or more R⁵². In one embodiment of Formula (S-I), (S-II), or (S-IV), including any of the foregoing, R^{2b} is —CH₂—R⁵¹ wherein R⁵¹ is 3- to 12-membered heterocycle optionally substituted with one or more R⁵² and R⁵² is C₁₋₆ alkyl. In one embodiment of Formula (S-I), (S-II), or (S-IV), including any of the foregoing, R^{2b} is —CH₂—R⁵¹ wherein R⁵¹ is 5- to 6-membered heterocycle optionally substituted with one or more R⁵² and R⁵² is C₁₋₆ alkyl. In one embodiment of Formula (S-I), (S-II), or (S-IV), including any of the foregoing, R^{2b} is —CH₂—R⁵¹ wherein R⁵¹ is an optionally substituted pyrazole, an optionally substituted oxazole, or an optionally substituted thiazole.

In one embodiment of Formula (S-I) or (S-II), including any of the foregoing, R^{2a} is optionally substituted C₁₋₆ alkyl, wherein the C₁₋₆ alkyl is optionally substituted with one R⁵¹ and R⁵¹ is 3- to 12-membered heterocycle optionally substituted with one or more R⁵² and R^{2b} is a 3- to 12-membered heterocycle containing at least one N atom and the heterocycle is further substituted with at least one R⁵³. In one embodiment of Formula (S-I) or (S-II), including any of the foregoing, R^{2a} is optionally substituted C₁₋₆ alkyl, wherein the C₁₋₆ alkyl is optionally substituted with one R⁵¹ and R⁵¹ is 5- to 6-membered heterocycle optionally substituted with one or more R⁵² and R^{2b} is a 5- to 6-membered heterocycle containing at least one N atom and the heterocycle is further substituted with at least one R⁵³. In one embodiment of Formula (S-I) or (S-II), including any of the foregoing, R^{2a} is optionally substituted C₁₋₆ alkyl, wherein the C₁₋₆ alkyl is optionally substituted with one R⁵¹ and R⁵¹ is an optionally substituted pyrazole, an optionally substituted oxazole, or an optionally substituted thiazole and R^{2b} is an optionally substituted pyrazole, an optionally substituted oxazole, or an optionally substituted thiazole.

In one embodiment of Formula (S-I) or (S-II), including any of the foregoing, R^{2b} is optionally substituted C₁₋₆ alkyl, wherein the C₁₋₆ alkyl is optionally substituted with one R⁵ and R⁵¹ is 3- to 12-membered heterocycle optionally substituted with one or more R⁵² and R^{2a} is a 3- to 12-membered heterocycle containing at least one N atom and the heterocycle is further substituted with at least one R⁵³. In one embodiment of Formula (S-I) or (S-II), including any of the foregoing, R^{2b} is optionally substituted C₁₋₆ alkyl, wherein the C₁₋₆ alkyl is optionally substituted with one R⁵¹ and R⁵¹ is 5- to 6-membered heterocycle optionally substituted with one or more R⁵² and R^{2a} is a 5- to 6-membered heterocycle containing at least one N atom and the heterocycle is further substituted with at least one R⁵³. In one embodiment of Formula (S-I) or (S-II), including any of the foregoing, R^{2b} is optionally substituted C₁₋₆ alkyl, wherein the C₁₋₆ alkyl is optionally substituted with one R⁵¹ and an optionally substituted pyrazole, an optionally substituted oxazole, or an optionally substituted thiazole and R^{2a} is an optionally substituted pyrazole, an optionally substituted oxazole, or an optionally substituted thiazole.

In one embodiment of Formula (S-I) or (S-II), including any of the foregoing, R^{2a} and/or R^{2b} are independently selected from

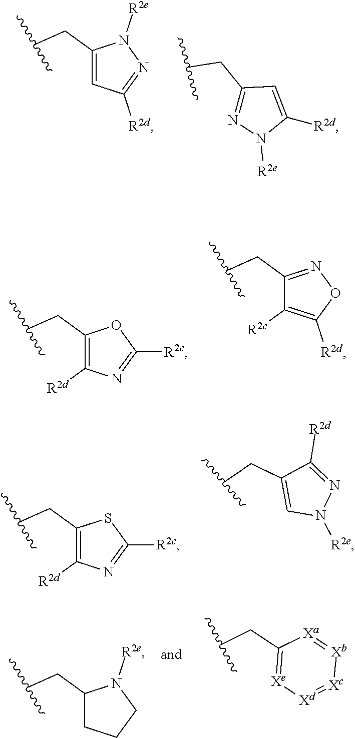

wherein R^{2c}, R^{2d}, R^{2e} and X^{a}-X^{e} are as defined herein.

In one embodiment of Formula (S-I) or (S-II), including any of the foregoing, R^{2b} is selected from

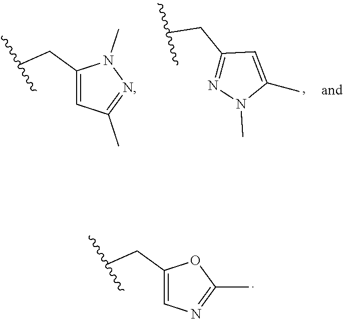

In one embodiment of Formula (S-I) or (S-II), including any of the foregoing, R^{2b} is selected from

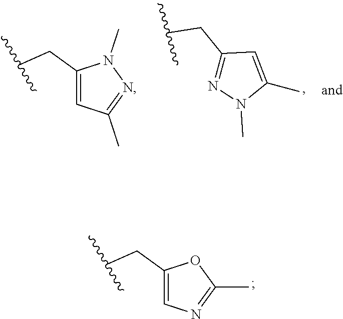

and R^{1b} is hydrogen.

In one embodiment of Formula (S-I) or (S-II), including any of the foregoing, R^{2a} is selected from

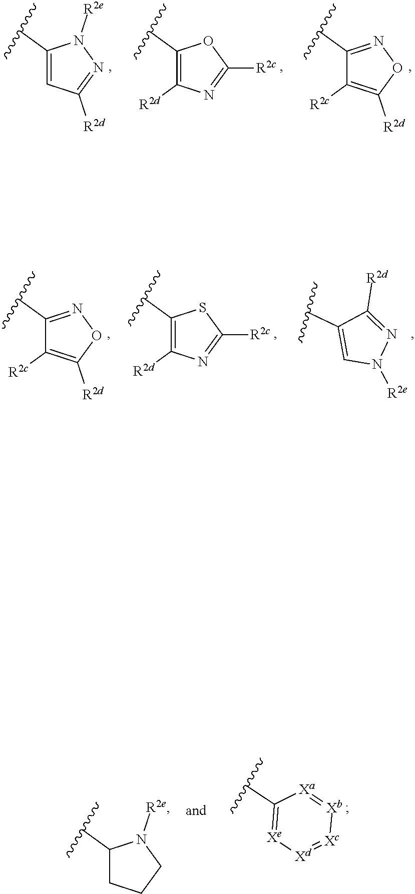

and R^{2b} is selected from

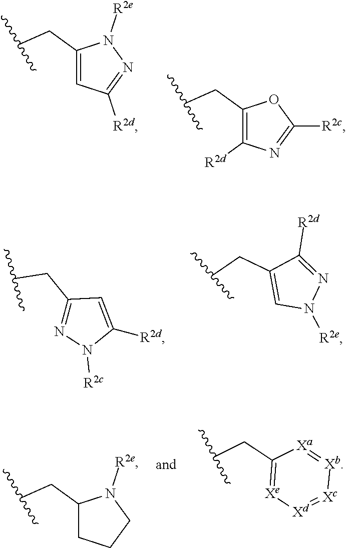

In one embodiment of Formula (S-I) or (S-II), including any of the foregoing, R^{2a} is selected from

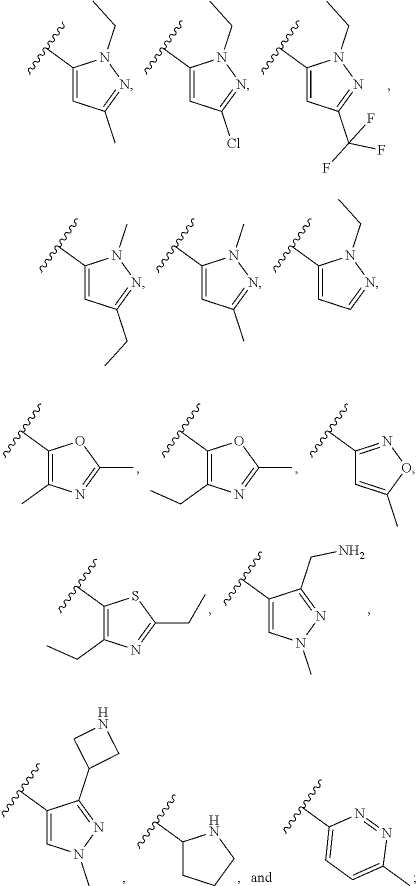

and R^{2b} is selected from

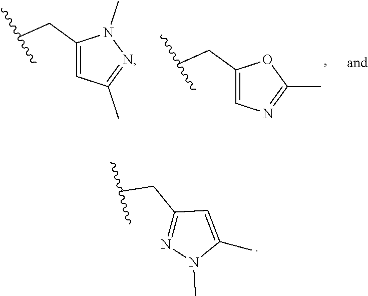

In one embodiment of Formula (S-I) or (S-II), including any of the foregoing, R^{2a} is

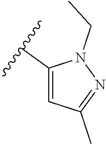 [SMILES: *c1cc(C)nn1CC]

and R^{2b} is selected from

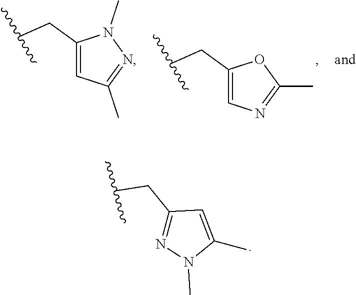

In one embodiment of Formula (S-I) or (S-II), including any of the foregoing, R^{2a} is

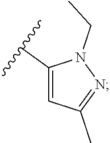 [SMILES: *c1cc(C)nn1CC]

R^{2b} is selected from

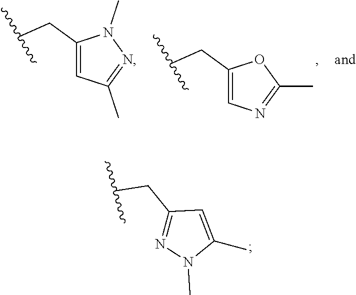

and R^{1a} and R^{1b} are both hydrogen.

In one embodiment of Formula (S-I) or (S-II), including any of the foregoing, R^{1b} and R^{2b} are joined together with the atoms to which they are attached to form an optionally substituted 3- to 12-membered heterocycle wherein the heterocycle is optionally substituted with one or more R⁵³. In one embodiment of Formula (S-I) or (S-II), including any of the foregoing, R^{1b} and R^{2b} are joined together with the atoms to which they are attached to form an optionally substituted 8- to 12-membered heterocycle wherein the heterocycle is optionally substituted with one or more R⁵³. In one embodiment of Formula (S-I) or (S-II), including any of the foregoing, R^{1b} and R^{2b} are joined together with the atoms to which they are attached to form an optionally substituted N—C(O)-linked 3- to 12-membered fused heterocycle. In one embodiment of Formula (S-I) or (S-II), including any of the foregoing, R^{1b} and R^{2b} are joined together with the atoms to which they are attached to form an optionally substituted N—C(O)-linked 8- to 12-membered fused heterocycle.

In one embodiment of Formula (S-I) or (S-II), including any of the foregoing, R^{1a} and R^{2a} are joined together with the atoms to which they are attached to form an optionally substituted 3- to 12-membered heterocycle wherein the heterocycle is optionally substituted with one or more R⁵³. In one embodiment of Formula (S-I) or (S-II), including any of the foregoing, R^{1a} and R^{2a} are joined together with the atoms to which they are attached to form an optionally substituted 8- to 12-membered heterocycle wherein the heterocycle is optionally substituted with one or more R⁵³. In one embodiment of Formula (S-I) or (S-II), including any of the foregoing, R^{1a} and R^{2a} are joined together with the atoms to which they are attached to form an optionally substituted N—C(O)-linked 3- to 12-membered fused heterocycle. In one embodiment of Formula (S-I) or (S-II), including any of the foregoing, R^{1a} and R^{2a} are joined together with the atoms to which they are attached to form an optionally substituted N—C(O)-linked 8- to 12-membered fused heterocycle.

In one embodiment of Formula (S-I) or (S-II), including any of the foregoing, R^{1b} and R^{2b} are joined together with the atoms to which they are attached to form an optionally substituted 3- to 12-membered heterocycle wherein the heterocycle is optionally substituted with one or more R⁵³, R^{1a} is hydrogen or C₁₋₆ alkyl, and R^{2a} is a 3- to 12-membered heterocycle, which is optionally substituted with one or more R⁵³. In one embodiment of Formula (S-I) or (S-II), including any of the foregoing, R^{1a} and R^{2a} are joined together with the atoms to which they are attached to form an optionally substituted 3- to 12-membered heterocycle wherein the heterocycle is optionally substituted with one or more R⁵³, R^{1b} is hydrogen or C₁₋₆ alkyl, and R^{2b} is a 3- to 12-membered heterocycle, which is optionally substituted with one or more R⁵³. In one embodiment of Formula (S-I) or (S-II), including any of the foregoing, R^{1b} and R^{2b} are joined together with the atoms to which they are attached to form an optionally substituted 3- to 12-membered heterocycle wherein the heterocycle is optionally substituted with one or more R⁵³ and R^{1a} and R^{2a} are joined together with the atoms to which they are attached to form an optionally substituted 3- to 12-membered heterocycle wherein the heterocycle is optionally substituted with one or more R⁵³.

In one embodiment of Formula (S-I) or (S-II), including any of the foregoing, the optionally substituted 3- to 12-membered heterocycle is an optionally substituted N—C(O)-linked 3- to 12-membered fused heterocycle. In one embodiment of Formula (S-I) or (S-II), including any of the foregoing, the optionally substituted 3- to 12-membered heterocycle is an optionally substituted N—C(O)-linked 8- to 12-membered fused heterocycle.

In one embodiment of Formula (S-III), including any of the foregoing, R^{9a} and R^{10a} are joined together with the atoms to which they are attached to form an optionally substituted 3- to 12-membered heterocycle wherein the heterocycle is optionally substituted with one or more R⁵³, R^{9b} is hydrogen or C₁₋₆ alkyl, and R^{10b} is a 3- to 12-membered heterocycle, which is optionally substituted with one or more R⁵³. In one embodiment of Formula (S-III), including any of the foregoing, R^{9b} and R^{10b} are joined together with the atoms to which they are attached to form an optionally substituted 3- to 12-membered heterocycle wherein the heterocycle is optionally substituted with one or more R⁵³, R^{9a} is hydrogen or C₁₋₆ alkyl, and R^{10a} is a 3- to 12-membered heterocycle, which is optionally substituted with one or more R⁵³. In one embodiment of Formula (S-III), including any of the foregoing, R^{9a} and R^{10a} are joined together with the atoms to which they are attached to form an optionally substituted 3- to 12-membered heterocycle wherein the heterocycle is optionally substituted with one or more R⁵³ and R^{9b} and R^{10b} are joined together with the atoms to which they are attached to form an optionally substituted 3- to 12-membered heterocycle wherein the heterocycle is optionally substituted with one or more R⁵³.

In one embodiment of Formula (S-III), including any of the foregoing, the optionally substituted 3- to 12-membered heterocycle is an optionally substituted N—C(O)-linked 3- to 12-membered fused heterocycle.

In one embodiment of Formula (S-I)—(S—V), including any of the foregoing, the optionally substituted N—C(O)-linked 3- to 12-membered fused heterocycle is an optionally substituted 5-5 fused ring system, an optionally substituted 5-6 fused ring system, an optionally substituted 6-6 fused ring system, or an optionally substituted 5-7 fused ring system.

In one embodiment of Formula (S-I) or (S-II), including any of the foregoing, R^{1a} is hydrogen; R^{2a} is

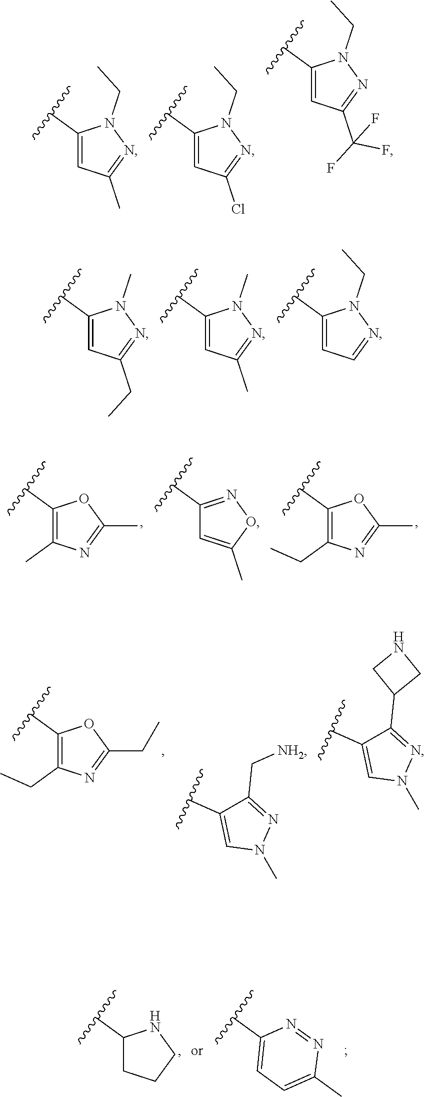

and R^{1b} and R^{2b} are joined together with the atoms to which they are attached to form

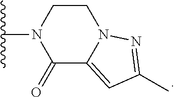 [SMILES: *N1CCn2nc(C)cc2C1=O]

In one embodiment of Formula (S-I) or (S-II), including any of the foregoing, R^{2a} is a C₃₋₁₂ carbocycle optionally substituted with one or more R⁵³. In one embodiment of Formula (S-I) or (S-II), including any of the foregoing, R^{2a} is a C₈₋₁₂ carbocycle optionally substituted with one or more R⁵³. In one embodiment of Formula (S-I) or (S-II), including any of the foregoing, R^{2b} is a C₃₋₁₂ carbocycle optionally substituted with one or more R⁵³. In one embodiment of Formula (S-I) or (S-II), including any of the foregoing, R^{2b} is a C₈₋₁₂ carbocycle optionally substituted with one or more R⁵³. In one embodiment of Formula (S-I) or (S-II), including any of the foregoing, the C₃₋₁₂ carbocycle is an optionally substituted bicyclic C₃₋₁₂ carbocycle, and can be bridged, fused, or spirocyclic. In one embodiment of Formula (S-I) or (S-II), including any of the foregoing, the C₈₋₁₂ carbocycle is an optionally substituted bicyclic C₃₋₁₂ carbocycle, and can be bridged, fused, or spirocyclic. In one embodiment of Formula (S-I) or (S-II), including any of the foregoing, R^{2a} and/or R^{2b} is an optionally substituted bridged C₃₋₁₂ carbocycle substituted with one R⁵³ and R⁵³ is C₁₋₆ alkyl. In one embodiment of Formula (S-I) or (S-II), including any of the foregoing, R^{2a} and/or R^{2b} is an optionally substituted bridged C₈₋₁₂ carbocycle substituted with one R⁵³ and R⁵³ is C₁₋₆ alkyl.

In one embodiment of Formula (S-I)-(S-IV), including any of the foregoing, L¹ is —C₂₋₁₀alkenyl- optionally substituted with one or more R⁵⁰. In one embodiment of Formula (S-I)-(S-IV), including any of the foregoing, L¹ is —CH═CH— optionally substituted with one R⁵⁰. In one embodiment of Formula (S-I)-(S-IV), including any of the foregoing, L¹ is —CH═CH—.

In one embodiment of Formula (S-I)-(S-IV), including any of the foregoing, L¹ is —C₁₋₁₀alkyl- optionally substituted with one or more R⁵⁰. In one embodiment of Formula (S-I)-(S-IV), including any of the foregoing, L¹ is —CH₂—CH₂— optionally substituted with one R⁵⁰. In one embodiment of Formula (SI)—(SIV), including any of the foregoing, L¹ is —CH₂—CH₂—.

In one embodiment of Formula (S-I)-(S-IV), including any of the foregoing, L¹ is

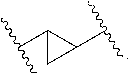 [SMILES: *C1CC1*]

In one embodiment of Formula (S-I), (S-III), (S-IV), including any of the foregoing, L² is C₁₋₆ alkyl. In one embodiment of Formula (S-I), (S-III), (S-IV), including any of the foregoing, L² is C₃₋₆ alkylene. In one embodiment of Formula (S-I), (S-III), (S-IV), including any of the foregoing, L² is —(CH₂)₃—. In one embodiment of Formula (S-I), (S-III), (S-IV), including any of the foregoing, L² is C₁₋₆ alkylene optionally substituted with one R⁵⁰.

In one embodiment, Formula (S-I) is of Formula (S-IA):

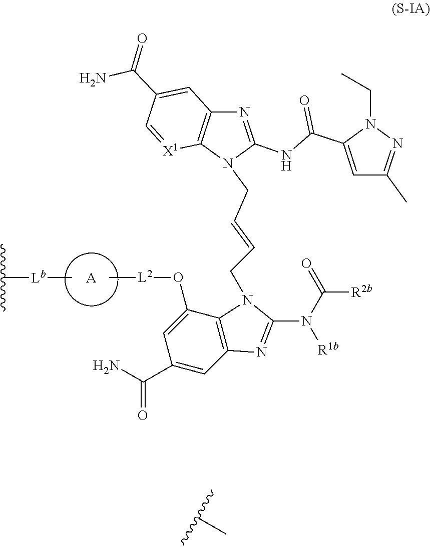

is the point of attachment to the rest of the formula; or a pharmaceutically acceptable salt or tautomer thereof.

In one embodiment of Formula (S-IA), L² is —(CH₂)₃—; R^{1b} is hydrogen; and R^{2b} is

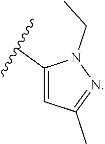 [SMILES: *c1cc(C)nn1CC]

In one embodiment of Formula (S-IA), L² is —(CH₂)₃—; R^{1b} is hydrogen; and R^{2b} is

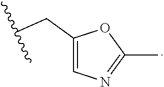 [SMILES: *Cc1cnc(C)o1]

In one embodiment of Formula (S-IA), L² is —(CH₂)₃—; R^{1b} is hydrogen.

In one embodiment, Formula (S-II) is of Formula (S-IIA) or (S-IIB):

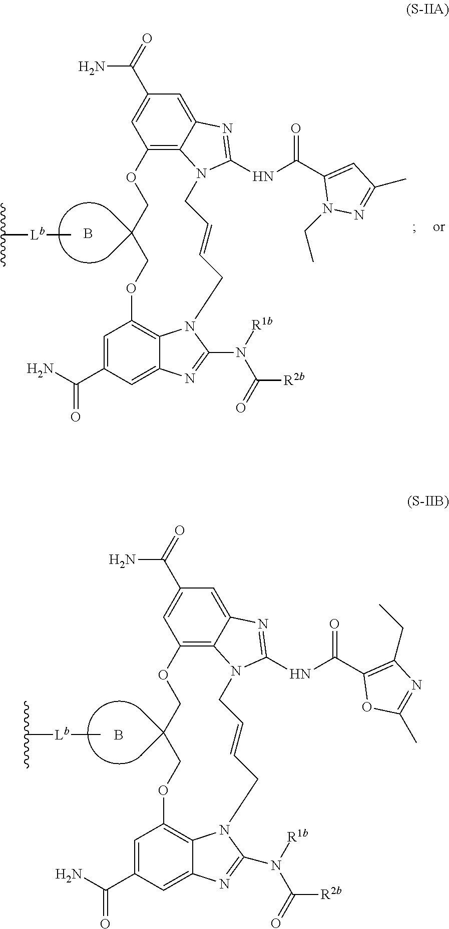

is the point of attachment to the rest of the formula; or a pharmaceutically acceptable salt or tautomer thereof.

In one embodiment of Formula (S-IIA), R^{1b} is hydrogen; and R^{2b} is

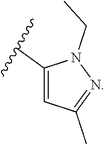 [SMILES: *c1cc(C)nn1CC]

In one embodiment of Formula (S-IB), R^{1b} is hydrogen; and R^{2b} is

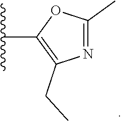 [SMILES: *c1oc(C)nc1CC]

In one embodiment of Formula (S-IIB), R^{1b} is hydrogen; and R^{2b} is

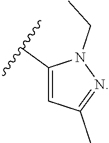 [SMILES: *c1cc(C)nn1CC]

R^{1b} is hydrogen; and R^{2b} is

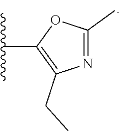 [SMILES: *c1oc(C)nc1CC]

In one embodiment, Formula (S-IV) is of Formula (S-IVA):

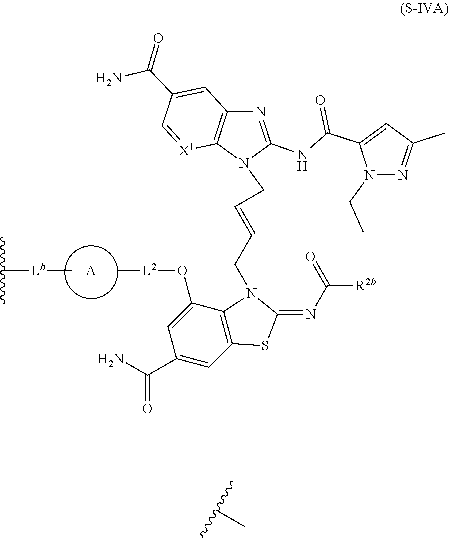

is the point of attachment to the rest of the formula; or a pharmaceutically acceptable salt or tautomer thereof.

In one embodiment of Formula (S-IVA), L² is —(CH₂)₃— and R^{2b} is

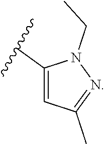 [SMILES: *c1cc(C)nn1CC]

In one embodiment, Formula (S-I) is of Formula (S-IC-1):

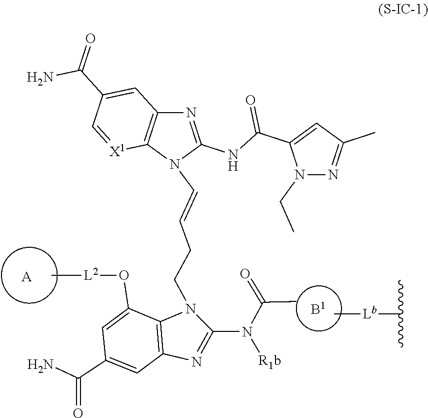

wherein B¹ optionally substituted C₃₋₁₂ carbocycle or optionally substituted 3- to 12-membered heterocycle, each of which is optionally substituted with one or more R⁵²; and
[symbol] is the point of attachment to the rest of the formula; or a pharmaceutically acceptable salt or tautomer thereof.

In one embodiment, Formula (S-I) is of Formula (S-IC-2):

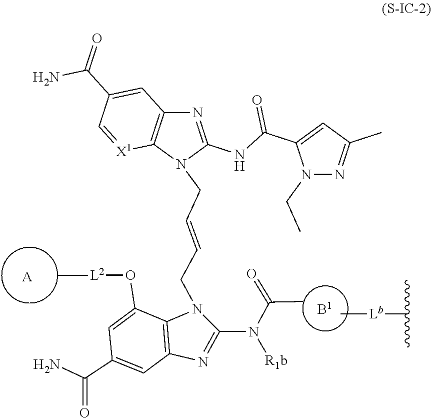

wherein B¹ optionally substituted C₃₋₁₂ carbocycle or optionally substituted 3- to 12-membered heterocycle, each of which is optionally substituted with one or more R⁵²; and
[symbol] is the point of attachment to the rest of the formula; or a pharmaceutically acceptable salt or tautomer thereof.

In one embodiment of Formula (S-IC-1) or (S-IC-2),

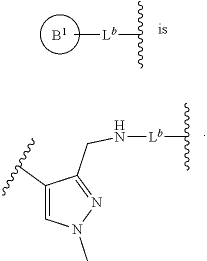

In one embodiment of Formula (S-I), L¹ is —CH₂CH₂—; R^{1a} and R^{1b} are both hydrogen; R^{2a} is

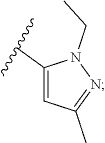 [SMILES: *c1cc(C)nn1CC]

### and R^{2b} is

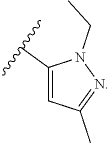 [SMILES: *c1cc(C)nn1CC]

In one embodiment of Formula (S-I), L¹ is —CH═CH—; R²⁰ is hydrogen; R^{1a} and R^{1b} are both hydrogen; and R^{2a} and R^{2b} are both

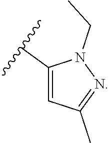 [SMILES: *c1cc(C)nn1CC]

In one embodiment of Formula (S-II), L¹ is —CH═CH—; R^{1a} and R^{1b} are both hydrogen and R^{2a} and R^{2b} are both

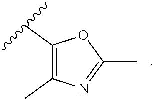 [SMILES: *c1oc(C)nc1C]

In one embodiment of Formula (S-I), (S-IA) (S-IB), (S-IC-1), (S-IC-2), (S-IV), or (S-IVA), including any of the foregoing, Ring A is an optionally substituted bridged, fused, or spirocyclic bicyclic heterocycle comprising at least one N atom and at least one O atom wherein the heterocycle is optionally substituted with one or more R⁵³. In one embodiment of Formula (S-I), (S-IA) (S-IB), (S-IC-1), (S-IC-2), (S-IV), or (S-IVA), including any of the foregoing, Ring A is an optionally substituted 3- to 12-membered bridged, fused, or spirocyclic bicyclic heterocycle comprising at least one nitrogen atom, including the nitrogen bound to L², and at least one oxygen atom. In one embodiment of Formula (S-I), (S-IA) (S-IB), (S-IC-1), (S-IC-2), (S-IV), or (S-IVA), including any of the foregoing, Ring A is an optionally substituted 8- to 12-membered bridged, fused, or spirocyclic bicyclic heterocycle comprising at least one nitrogen atom, including the nitrogen bound to L², and at least one oxygen atom. In one embodiment of Formula (S-I), (S-IA) (S-IB), (S-IC-1), (S-IC-2), (S-IV), or (S-IVA), including any of the foregoing, Ring A is an optionally substituted 3- to 12-membered N-linked bridged, fused, or spirocyclic bicyclic heterocycle comprising at least one nitrogen atom, including the nitrogen bound to L², and at least one oxygen atom. In one embodiment of Formula (S-I), (S-IA) (S-IB), (S-IC-1), (S-IC-2), (S-IV), or (S-IVA), including any of the foregoing, Ring A is an optionally substituted 8- to 12-membered N-linked bridged, fused, or spirocyclic bicyclic heterocycle comprising at least one nitrogen atom, including the nitrogen bound to L², and at least one oxygen atom. In one embodiment of Formula (S-I), (S-IA) (S-IB), (S-IC-1), (S-IC-2), (S-IV), or (S-IVA), including any of the foregoing, Ring A is an optionally substituted 3- to 12-membered N-linked spirocyclic bicyclic heterocycle comprising at least one nitrogen atom, including the nitrogen bound to L², and at least one oxygen atom. In one embodiment of Formula (S-I), (S-IA) (S-IB), (S-IC-1), (S-IC-2), (S-IV), or (S-IVA), including any of the foregoing, Ring A is an optionally substituted 3- to 12-membered N-linked spirocyclic bicyclic heterocycle comprising two nitrogen atoms, including the nitrogen bound to L² and a nitrogen bound to L^{b}, and one oxygen atom.

In one embodiment of Formula (S-I), (S-IA) (S-IB), (S-IC-1), (S-IC-2), (S-IV), or (S-IVA), including any of the foregoing, Ring A is selected from

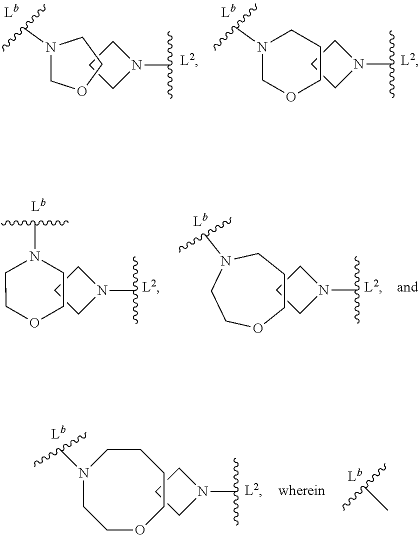

is the point of attachment to L^{b} and [symbol] is the point of attachment to L².

In one embodiment of Formula (S-I), (S-IA) (S-IB), (S-IC-1), (S-IC-2), (S-IV), or (S-IVA), including any of the foregoing, Ring A is selected from

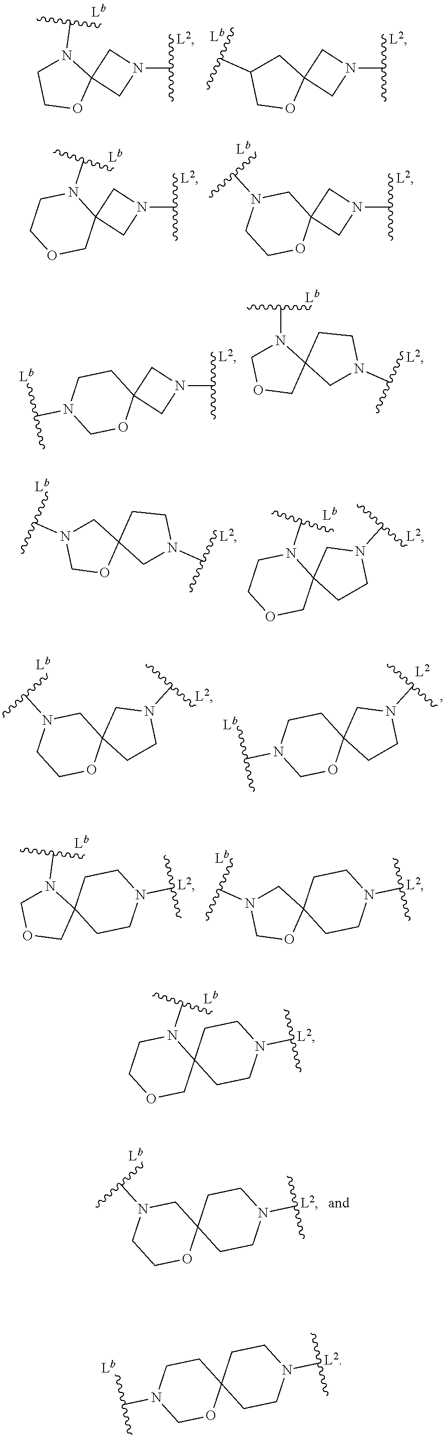

In one embodiment of Formula (S-I), (S-IA) (S-IB), (S-IC-1), (S-IC-2), (S-IV), or (S-IVA), including any of the foregoing, Ring A is selected from

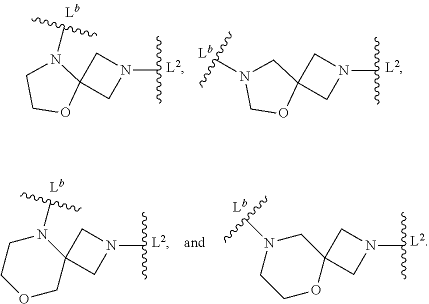 [SMILES: *N1CC2(C1)OCCN2* ; *N1COC2(C1)CN(*)C2 ; *N1CC2(COCCN2*)C1 ; *N1CCOC2(C1)CN(*)C2]

In one embodiment of Formula (S-I), (S-IA) (S-IB), (S-IC-1), (S-IC-2), (S-IV), or (S-IVA), including any of the foregoing, Ring A is selected from

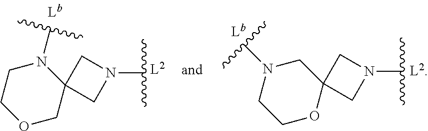 [SMILES: *N1CC2(COCCN2*)C1 ; *N1CCOC2(C1)CN(*)C2]

In one embodiment of Formula (S-I), (S-IA) (S-IB), (S-IC-1), (S-IC-2), (S-IV), or (S-IVA), including any of the foregoing, Ring A is

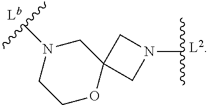 [SMILES: *N1CCOC2(C1)CN(*)C2]

In one embodiment of Formula (S-I), (S-IA) (S-IB), (S-IC-1), (S-IC-2), (S-IV), or (S-IVA), including any of the foregoing, Ring A is a 3- to 12-membered heterocycle substituted with R⁴. In one embodiment of Formula (S-I), (S-IA) (S-IB), (S-IC-1), (S-IC-2), (S-IV), or (S-IVA), including any of the foregoing, Ring A is a 8- to 12-membered heterocycle substituted with R⁴.

In one embodiment of Formula (S-I), (S-IA) (S-IB), (S-IC-1), (S-IC-2), (S-IV), or (S-IVA), including any of the foregoing, Ring A is a N-linked monocyclic 3- to 12-membered heterocycle comprising the N to which the ring is attached wherein the heterocycle is substituted with R⁴. In one embodiment of Formula (S-I), (S-IA) (S-IB), (S-IC-1), (S-IC-2), (S-IV), or (S-IVA), including any of the foregoing, Ring A is a N-linked monocyclic 8- to 12-membered heterocycle comprising the N to which the ring is attached and a second NH and wherein the heterocycle is substituted with R⁴.

In one embodiment of Formula (S-I), (S-IA) (S-IB), (S-IC-1), (S-IC-2), (S-IV), or (S-IVA), including any of the foregoing, Ring A is selected from

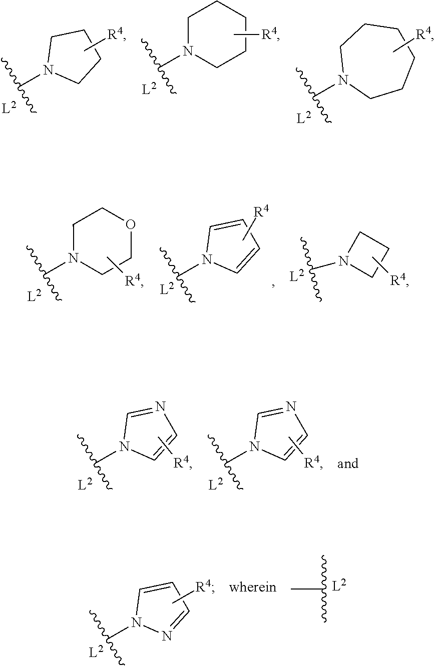

is the point of attachment to the rest of the compound.

In one embodiment of Formula (S-I), (S-IA) (S-IB), (S-IC-1), (S-IC-2), (S-IV), or (S-IVA), including any of the foregoing, Ring A is selected from

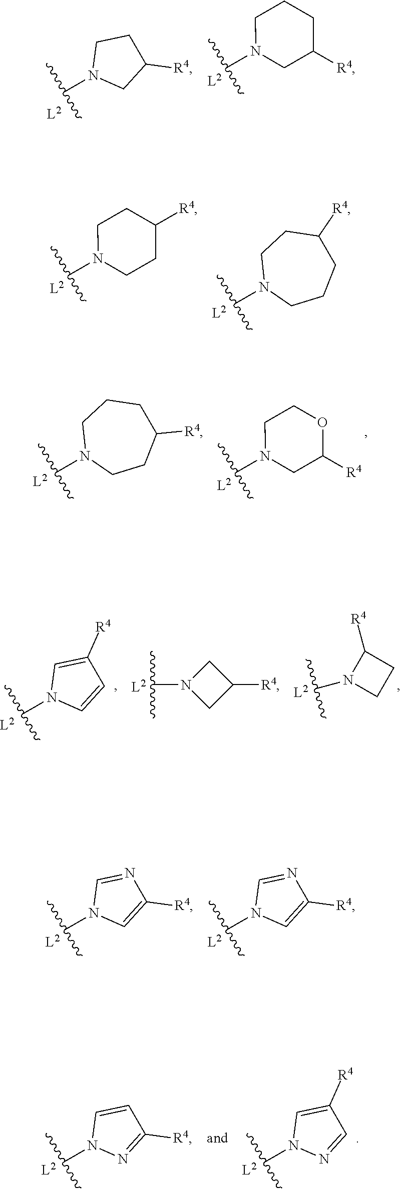

In one embodiment of Formula (S-I), (S-IA) (S—B), (S-IC-1), (S-IC-2), (S-IV), or (S-IVA), including any of the foregoing, Ring A is selected from

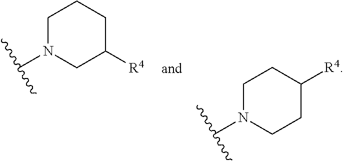

In one embodiment Formula (S-I), (S-IA) (S-IB), (S-IC-1), (S-IC-2), (S-IV), or (S-IVA), including any of the foregoing, Ring A is

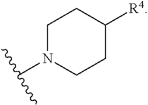

In one embodiment of Formula (S-I), (S-IA) (S-IB), (S-IC-1), (S-IC-2), (S-IV), or (S-IVA), including any of the foregoing, R⁴ is a 3- to 12-membered bridged or fused bicyclic heterocycle comprising 1, 2, 3, or 4 heteroatoms selected from N, S, and O. In one embodiment of Formula (S-I), (S-IA) (S-IB), (S-IC-1), (S-IC-2), (S-IV), or (S-IVA), including any of the foregoing, R⁴ is a 8- to 12-membered bridged or fused bicyclic heterocycle comprising 1, 2, 3, or 4 heteroatoms selected from N, S, and O. In one embodiment of Formula (S-I), (S-IA) (S-IB), (S-IC-1), (S-IC-2), (S-IV), or (S-IVA), including any of the foregoing, R⁴ is a 5-5 fused ring system, a 5-6 fused ring system, a 6-6 fused ring system, or a 5-7 fused ring system comprising 1, 2, 3, or 4 heteroatoms selected from N, S, and O. In one embodiment of Formula (S-I), (S-IA) (S-IB), (S-IC-1), (S-IC-2), (S-IV), or (S-IVA), including any of the foregoing, R⁴ is 5-6 fused ring system comprising 1, 2, 3, or 4 heteroatoms selected from N, S, and O.

In one embodiment of Formula (S-I), (S-IA) (S-IB), (S-IV), or (S-IVA), including any of the foregoing, R⁴ is a 3- to 12-membered bridged or fused bicyclic heterocycle comprising 1, 2, 3, or 4 heteroatoms selected from N, S, and O wherein at least one heteroatom is a N bound to -L^{b}. In one embodiment of Formula (S-I), (S-IA) (S-IB), (S-IV), or (S-IVA), including any of the foregoing, R⁴ is a 5-5 fused ring system, a 5-6 fused ring system, a 6-6 fused ring system, or a 5-7 fused ring system comprising 1, 2, 3, or 4 heteroatoms selected from N, S, and O wherein at least one heteroatom is a N bound to -L^{b}. In one embodiment of Formula (S-I), (S-IA) (S-IB), (S-IV), or (S-IVA), including any of the foregoing, R⁴ is a 5-6 fused ring system comprising 1, 2, 3, or 4 heteroatoms selected from N, S, and O wherein at least one heteroatom is a N bound to -L^{b}.

In one embodiment of Formula (S-I), (S-IA) (S-IB), (S-IV), or (S-IVA), including any of the foregoing, R⁴ is

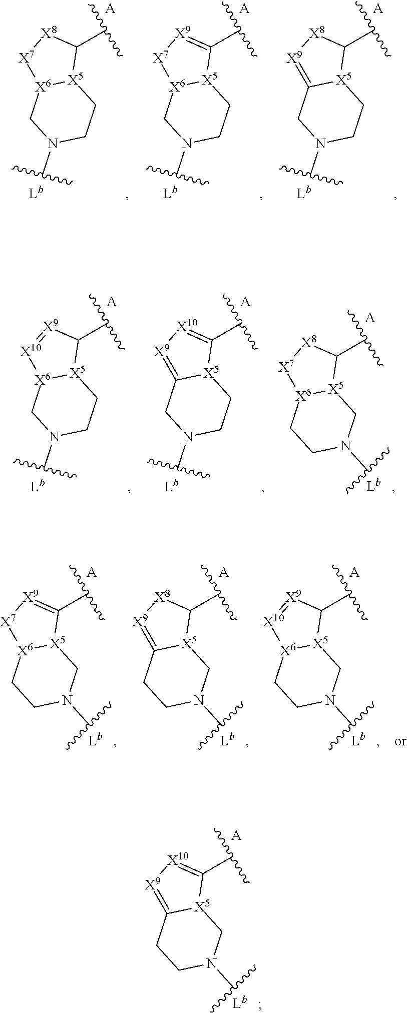

wherein X⁵, X⁶, X⁹, X¹⁰ are independently N or CR¹³;
X⁷ and X⁸ are independently NH, O, or CHR¹³;
R¹³ is independently hydrogen or C₁₋₆ alkyl;
R⁵ is as defined herein; and
[symbol] is the point of attachment to ring A and [symbol] is a bond to L^{b};
wherein if four of any one of X⁵—X¹⁰ are present, at least one is CR¹³ or CHR¹³.

In one embodiment of Formula (S-I), (S-IA) (S-IB), (S-IV), or (S-IVA), including any of the foregoing, R⁴ is

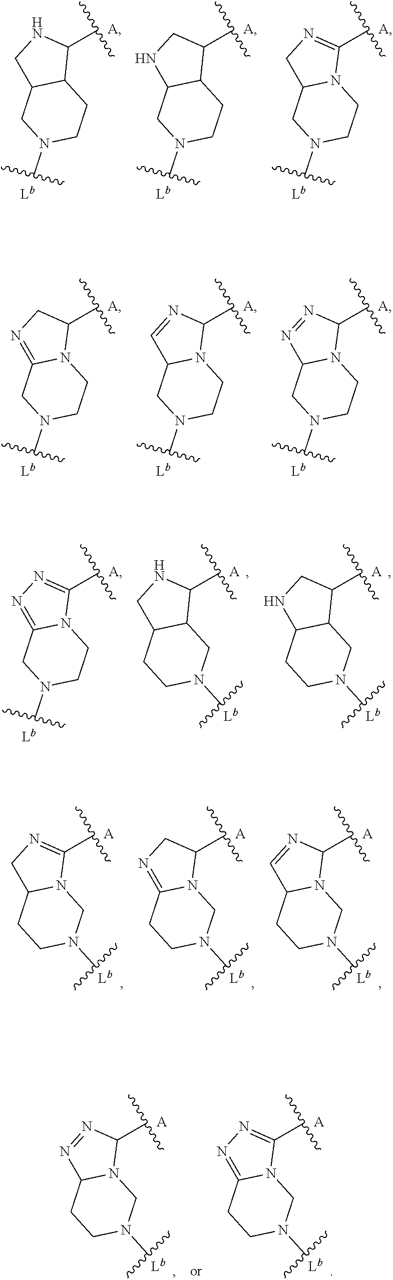

In one embodiment of Formula (S-I), (S-IA) (S-IB), (S-IV), or (S-IVA), including any of the foregoing, R⁴ is

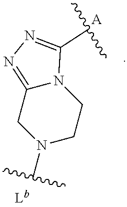

In one embodiment of Formula (S-I), (S-IA) (S-IB), (S-IV), or (S-IVA), including any of the foregoing, Ring A is

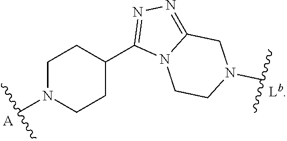

In one embodiment of Formula (S-III) or (S-IIIA), including any of the foregoing, Ring A¹ is an optionally substituted 3- to 12-membered heterocycle optionally substituted with one or more R⁵³. In one embodiment of Formula (S-III) or (S-IIIA), including any of the foregoing, Ring A¹ is an optionally substituted 8- to 12-membered heterocycle optionally substituted with one or more R⁵³.

In one embodiment of Formula (S-III) or (S-IIIA), including any of the foregoing, Ring A¹ is a N-linked monocyclic 3- to 12-membered heterocycle comprising a N bound to L² and a N bound to L^{b} and wherein the heterocycle is optionally substituted with one or more R⁵³. In one embodiment of Formula (S-III) or (S-IIIA), including any of the foregoing, Ring A¹ is a N-linked monocyclic 8- to 12-membered heterocycle comprising a N bound to L² and a N bound to L^{b} and wherein the heterocycle is optionally substituted with one or more R⁵³.

In one embodiment of Formula (S-IC-1) or (S-IC-2), including any of the foregoing, Ring B¹ is an optionally substituted C₃₋₁₂ carbocycle optionally substituted with one or more R⁵³. In one embodiment of Formula (S-IC-1) or (S-IC-2), including any of the foregoing, Ring B¹ is an optionally substituted C₆₋₁₂ carbocycle optionally substituted with one or more R⁵³. In one embodiment of Formula (S-IC-1) or (S-IC-2), including any of the foregoing, Ring B¹ is an optionally substituted 3- to 12-membered heterocycle optionally substituted with one or more R⁵³. In one embodiment of Formula (S-IC-1) or (S-IC-2), including any of the foregoing, Ring B¹ is an optionally substituted 5- to 6-membered heterocycle optionally substituted with one or more R⁵³. In one embodiment of Formula (S-IC-1) or (S-IC-2), including any of the foregoing, Ring B¹ is an optionally substituted 3- to 12-membered heterocycle comprising at least a N bound to -L^{b} and optionally substituted with one or more R⁵³. In one embodiment of Formula (S-IC-1) or (S-IC-2), including any of the foregoing, Ring B¹ is an optionally substituted 4- to 6-membered heterocycle comprising at least a N bound to -L^{b} and optionally substituted with one or more R⁵³. In one embodiment of Formula (S-IC-1) or (S-IC-2), including any of the foregoing, Ring B¹ is a 3- to 12-membered heterocycle comprising at least a N bound to -L^{b}. In one embodiment of Formula (S-IC-1) or (S-IC-2), including any of the foregoing, Ring B¹ is a 4- to 6-membered heterocycle comprising at least a N bound to -L^{b}.

In one embodiment of Formula (S-IC-1) or (S-IC-2), including any of the foregoing, Ring B¹ is selected from the group consisting of

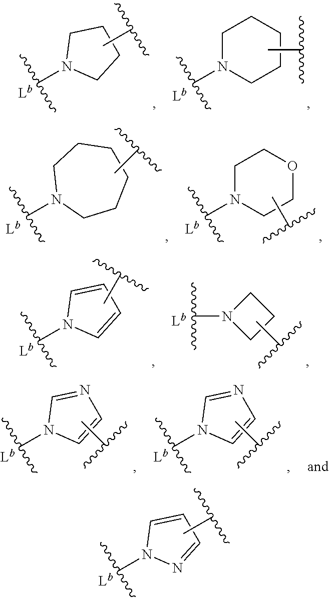

is the point of attachment to the rest of the compound and [symbol] is a bond to L^{b}.

Non-limiting examples of Formula (S-IA) include:

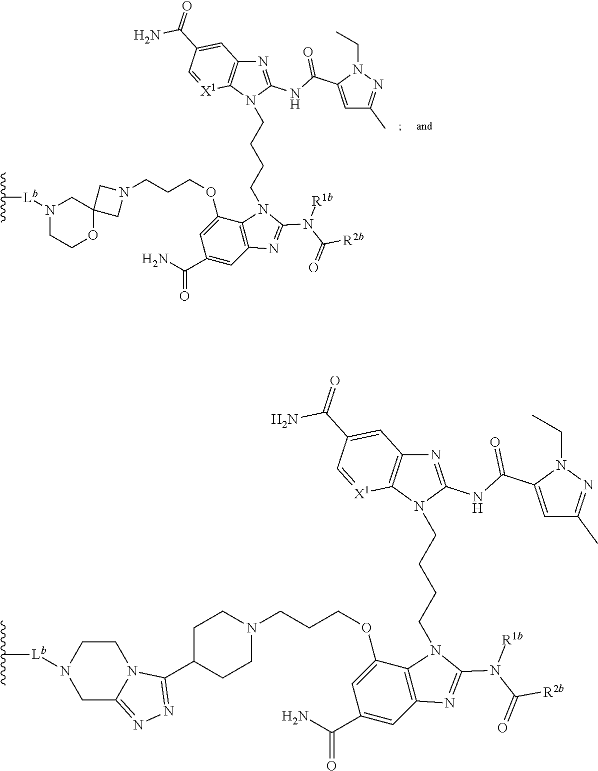

or a stereoisomer thereof.

Non-limiting examples of Formula (S-IIA), (S-IIB), or (S-IIC) include:

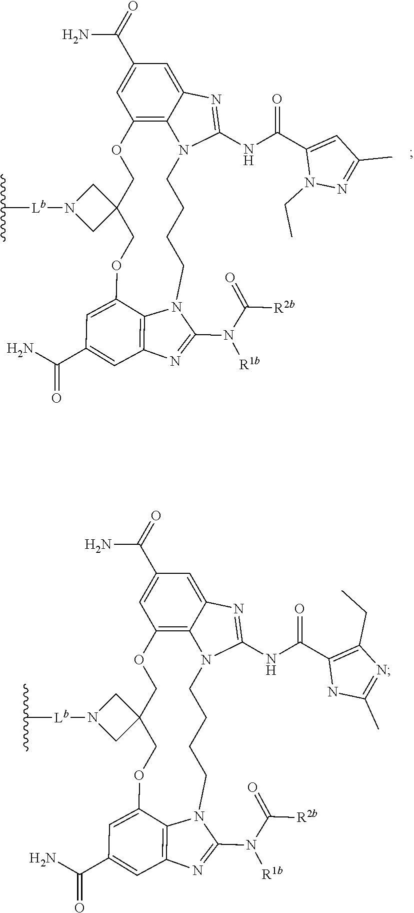

and
or a stereoisomer thereof.

Non-limiting examples of Formula (S-IVA) include:

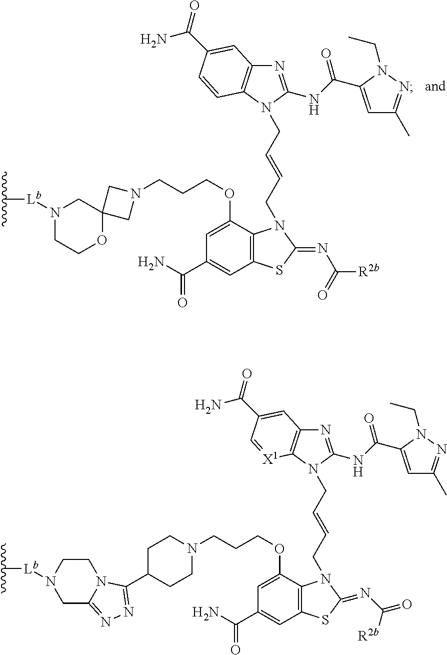

or a stereoisomer thereof.

Non-limiting examples of Formula (S-IC-1) include:

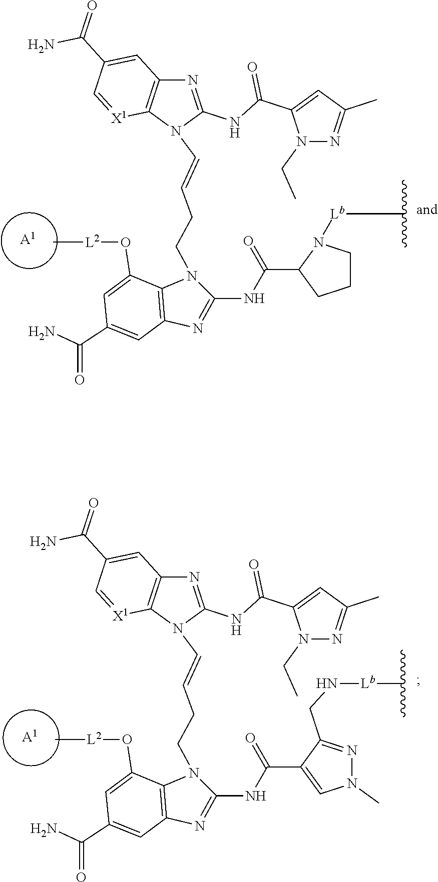

or a stereoisomer thereof.

Non-limiting examples of Formula (S-IC-2) include:

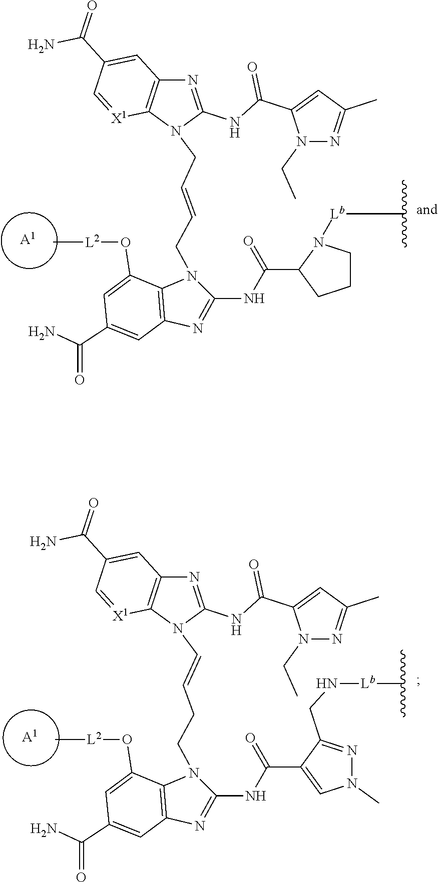

or a stereoisomer thereof.

In one embodiment of Formula (S-IA), L¹ is —CH═CH—; R^{1a} and R^{1b} are both hydrogen; Ring A is

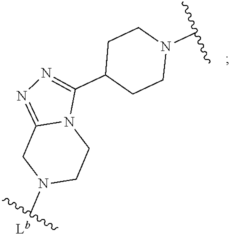 [SMILES: *N1CCC(c2nnc3n2CCN(*)C3)CC1]

### and R^{2b} is

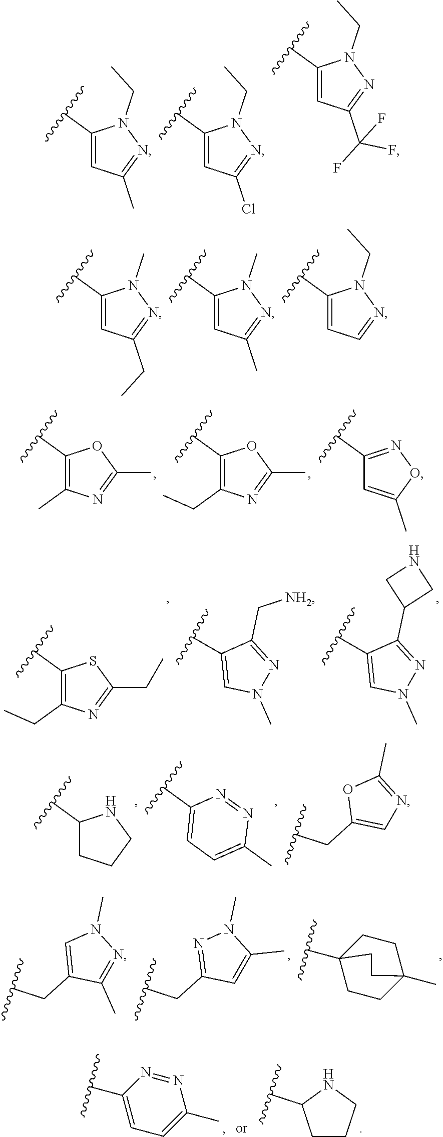

In one embodiment of Formula (S-IA), L¹ is —CH═CH—; R^{1a} and R^{1b} are both hydrogen; Ring A is

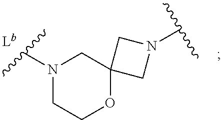 [SMILES: *N1CCOC2(C1)CN(*)C2]

### and R^{2b} is

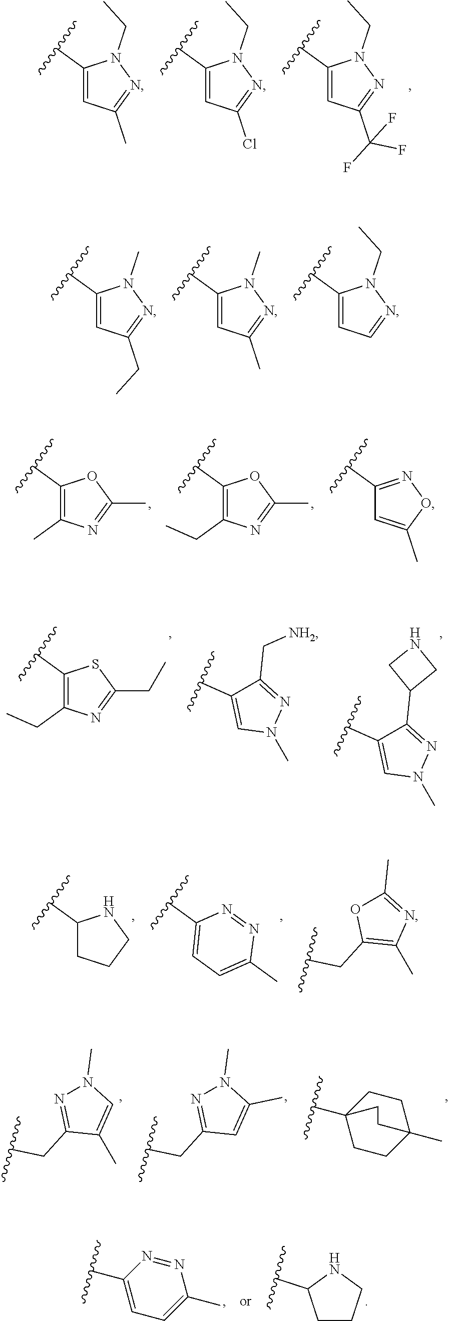

In one embodiment of Formula (S-IA), L¹ is —CH═CH—; R^{1a} is hydrogen; R^{1b} and R^{2b} are joined together to form

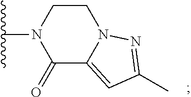 [SMILES: *N1CCn2nc(C)cc2C1=O]

### and Ring A is

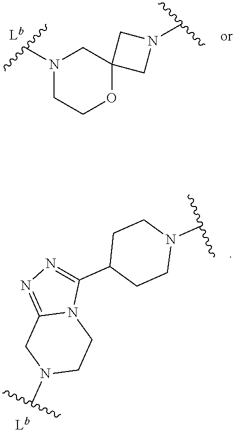 [SMILES: *N1CCOC2(C1)CN(*)C2 ; *N1CCC(c2nnc3n2CCN(*)C3)CC1]

In one embodiment of Formula (S-IVA), L¹ is —CH═CH—; R^{1a} is hydrogen; Ring A is

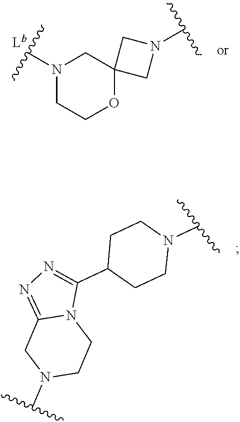 [SMILES: *N1CCOC2(C1)CN(*)C2 ; *N1CCC(c2nnc3n2CCN(*)C3)CC1]

### and R^{2b} is

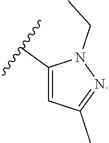 [SMILES: *c1cc(C)nn1CC]

In one embodiment of Formula (S-IA), L¹ is —CH═CH—; R^{1a} and R^{1b} are both hydrogen; R^{2a} is selected from

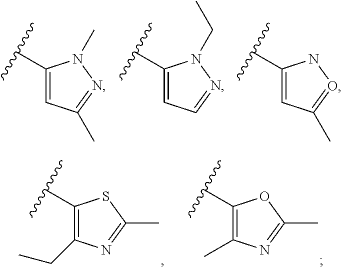

R^{2b} is selected from

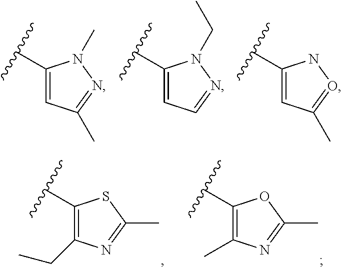

### and Ring A is

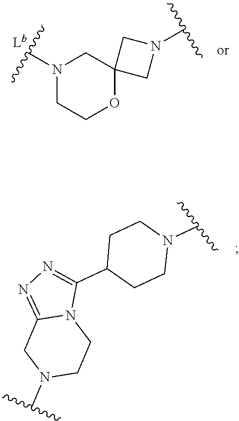 [SMILES: *N1CCOC2(C1)CN(*)C2 ; *N1CCC(c2nnc3n2CCN(*)C3)CC1]

In one embodiment of Formula (S-IA), L¹ is —CH₂CH₂—; R^{1a} and R^{1b} are both hydrogen; R^{2a} is

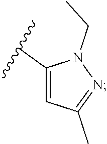 [SMILES: *c1cc(C)nn1CC]

### and R^{2b} is

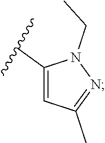 [SMILES: *c1cc(C)nn1CC]

### and Ring A is or

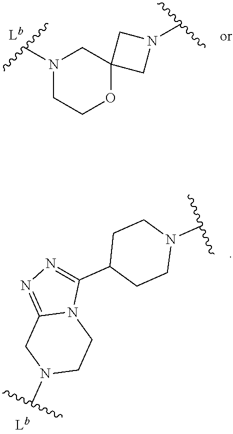 [SMILES: *N1CCOC2(C1)CN(*)C2 ; *N1CCC(c2nnc3n2CCN(*)C3)CC1]

In one embodiment of Formula (I),

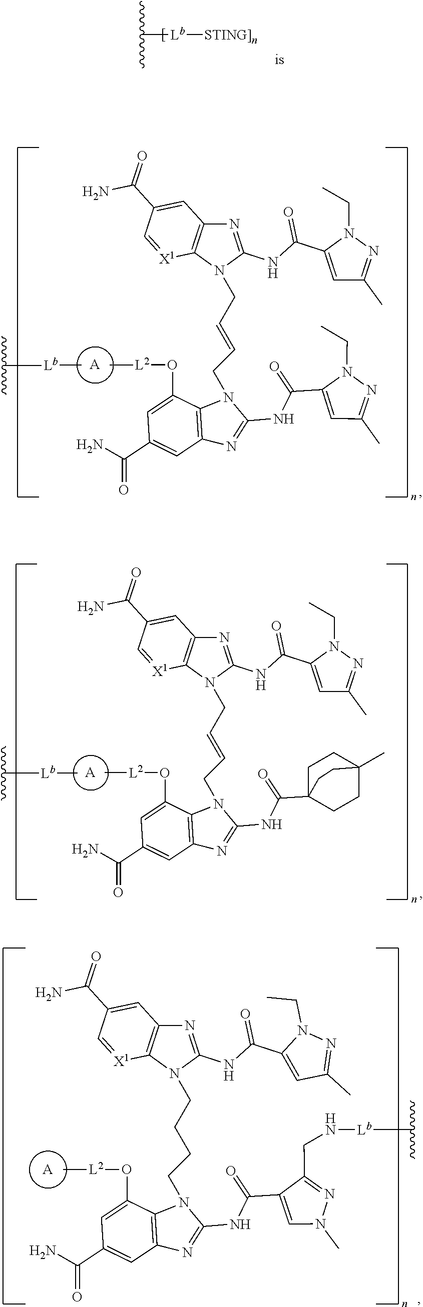

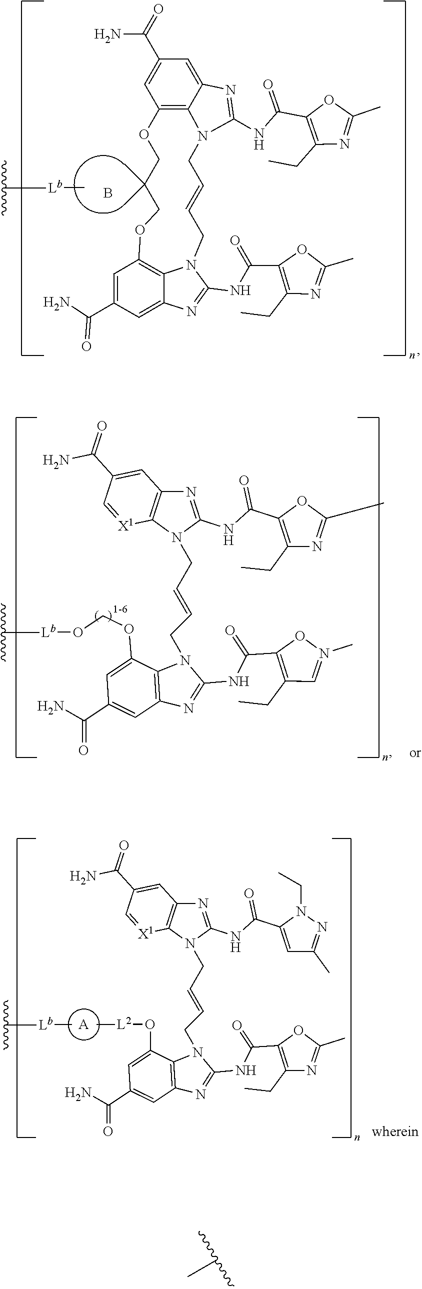

is the point of attachment to the rest of the formula.

In one embodiment of Formula (I), the STING agonist is a residue of a compound selected from:

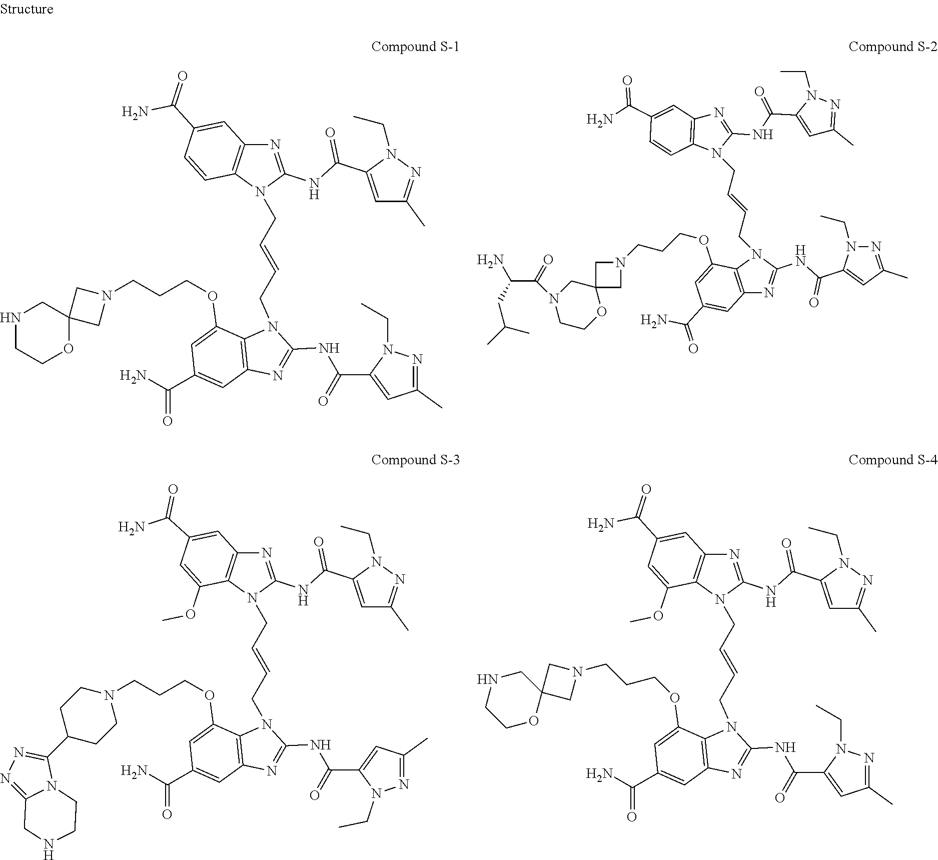 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCNC5)CC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(CNCCO4)C3)c21]

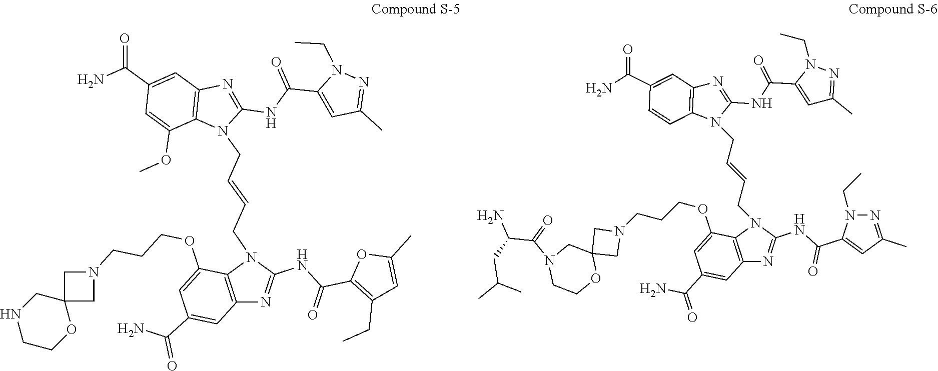 [SMILES: CCc1cc(C)oc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21]

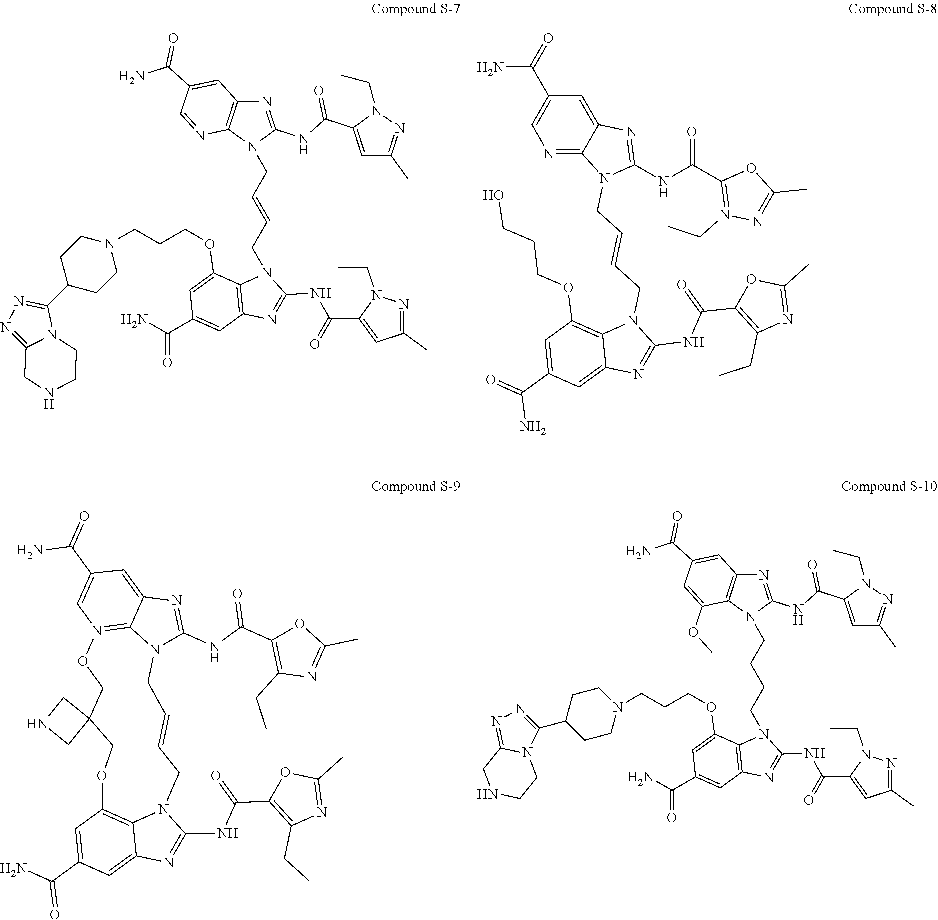

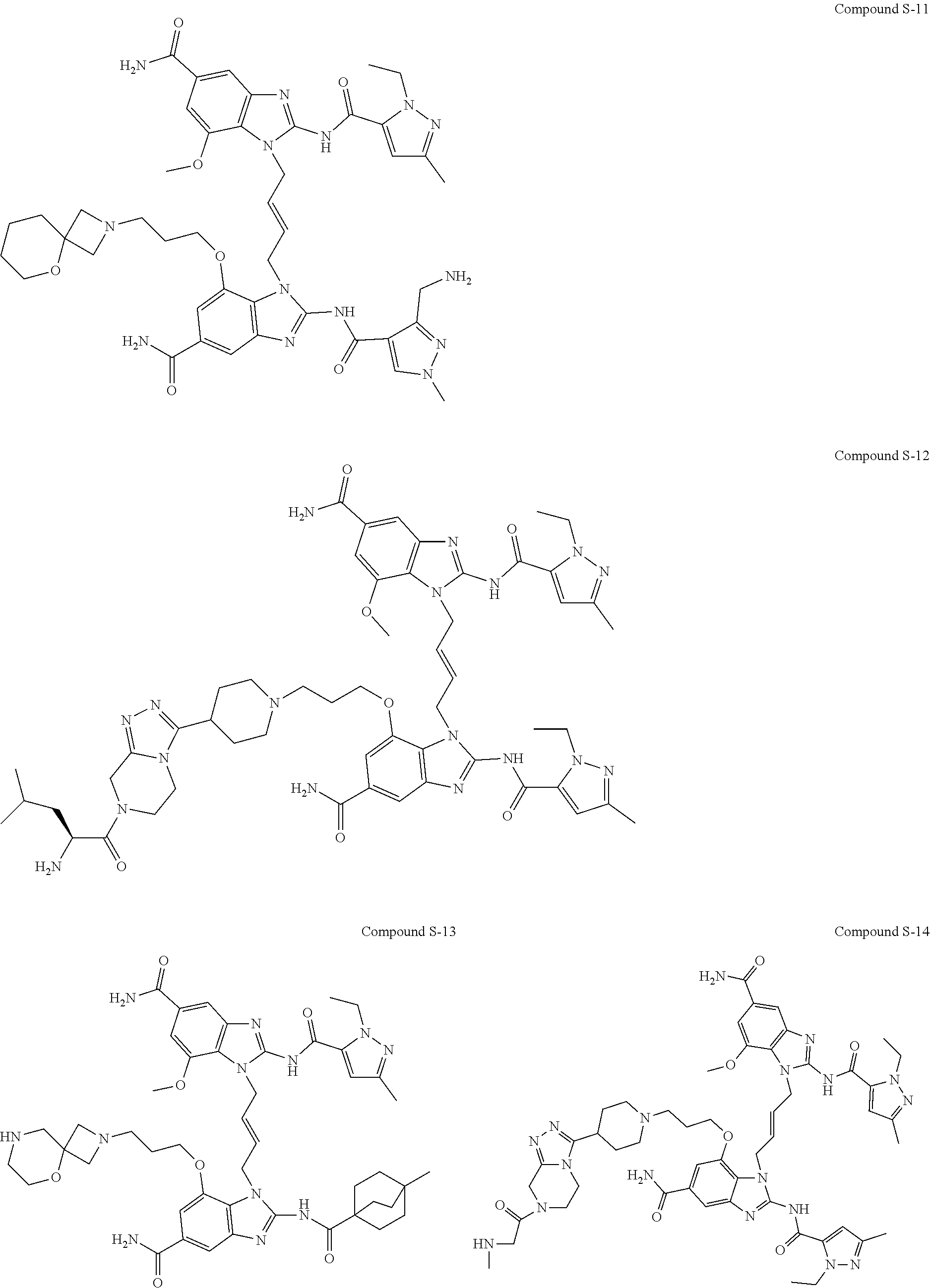

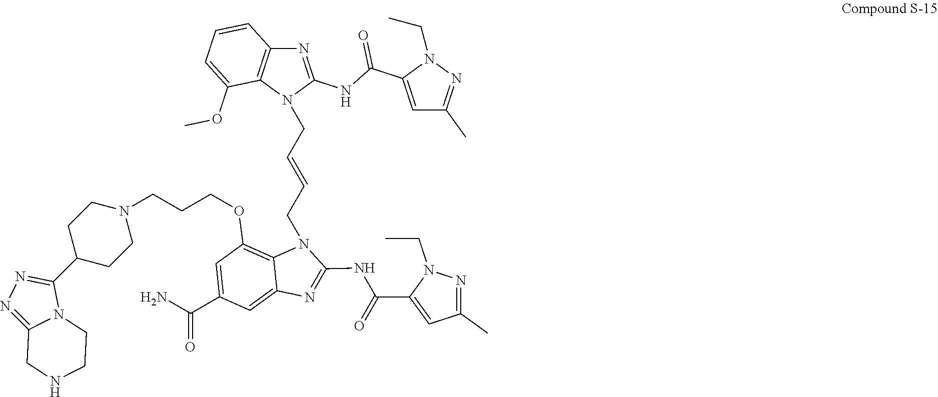 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCNC5)CC3)c21]

In one embodiment of Formula (I), the STING agonist is a residue of a compound selected from:

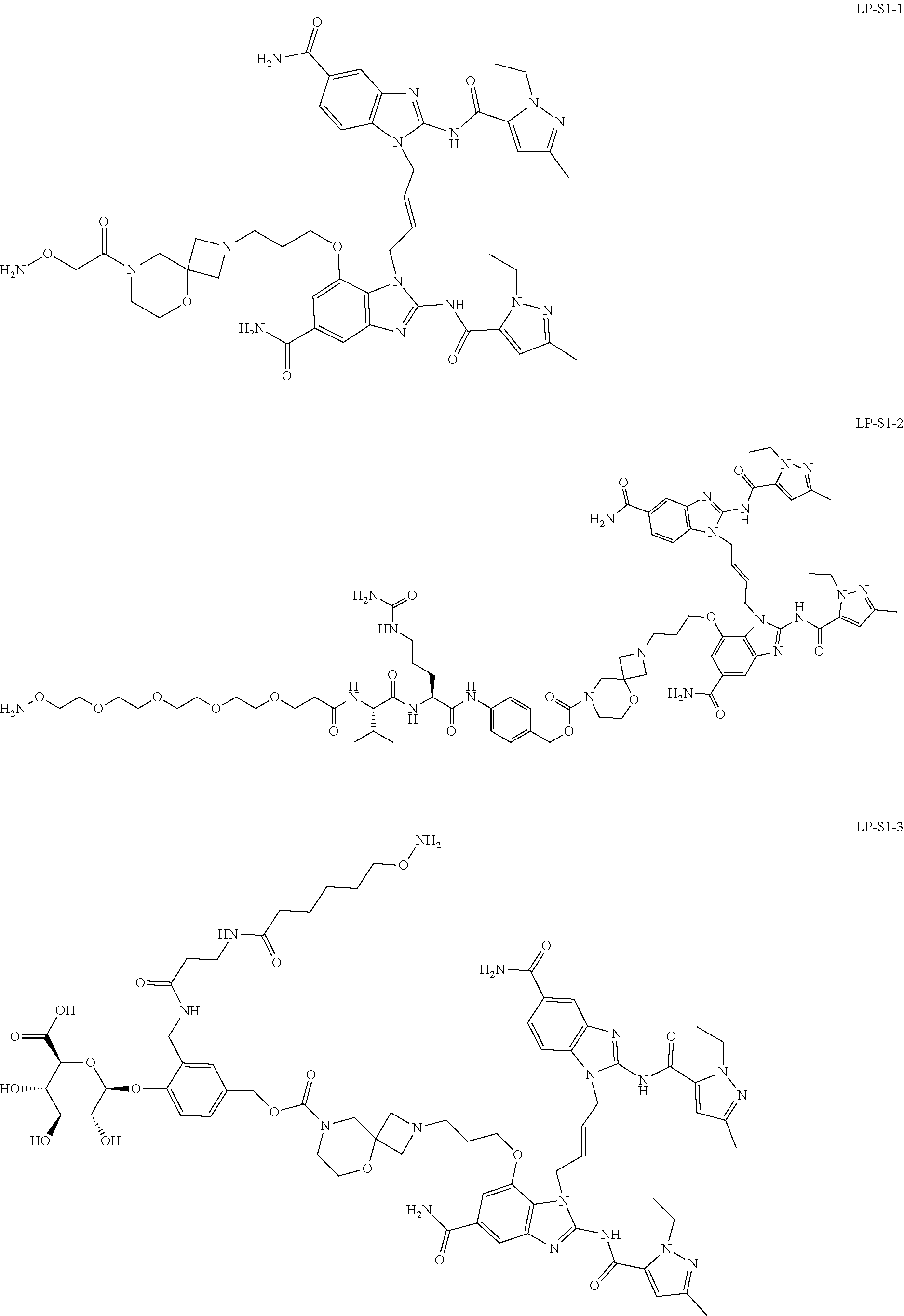

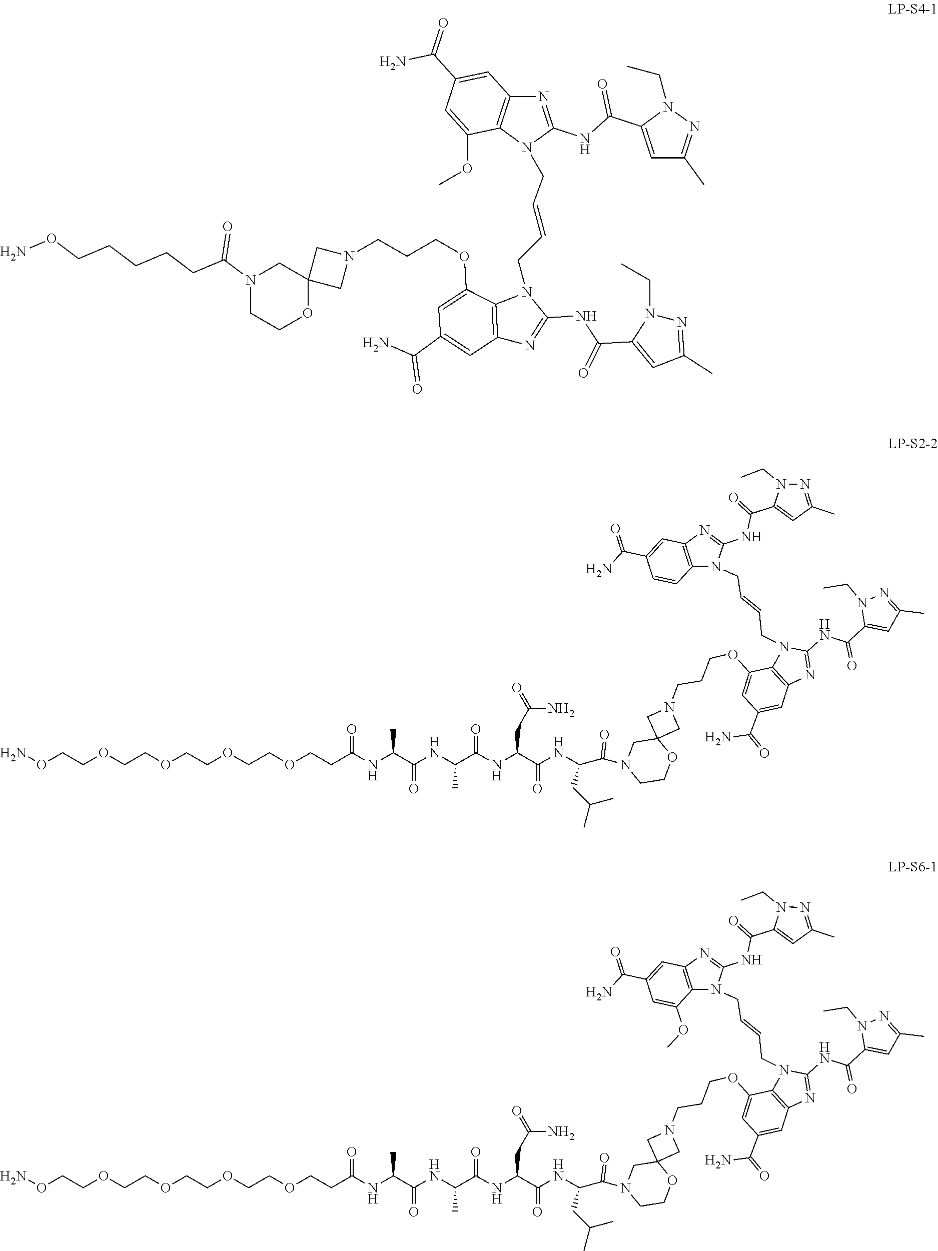

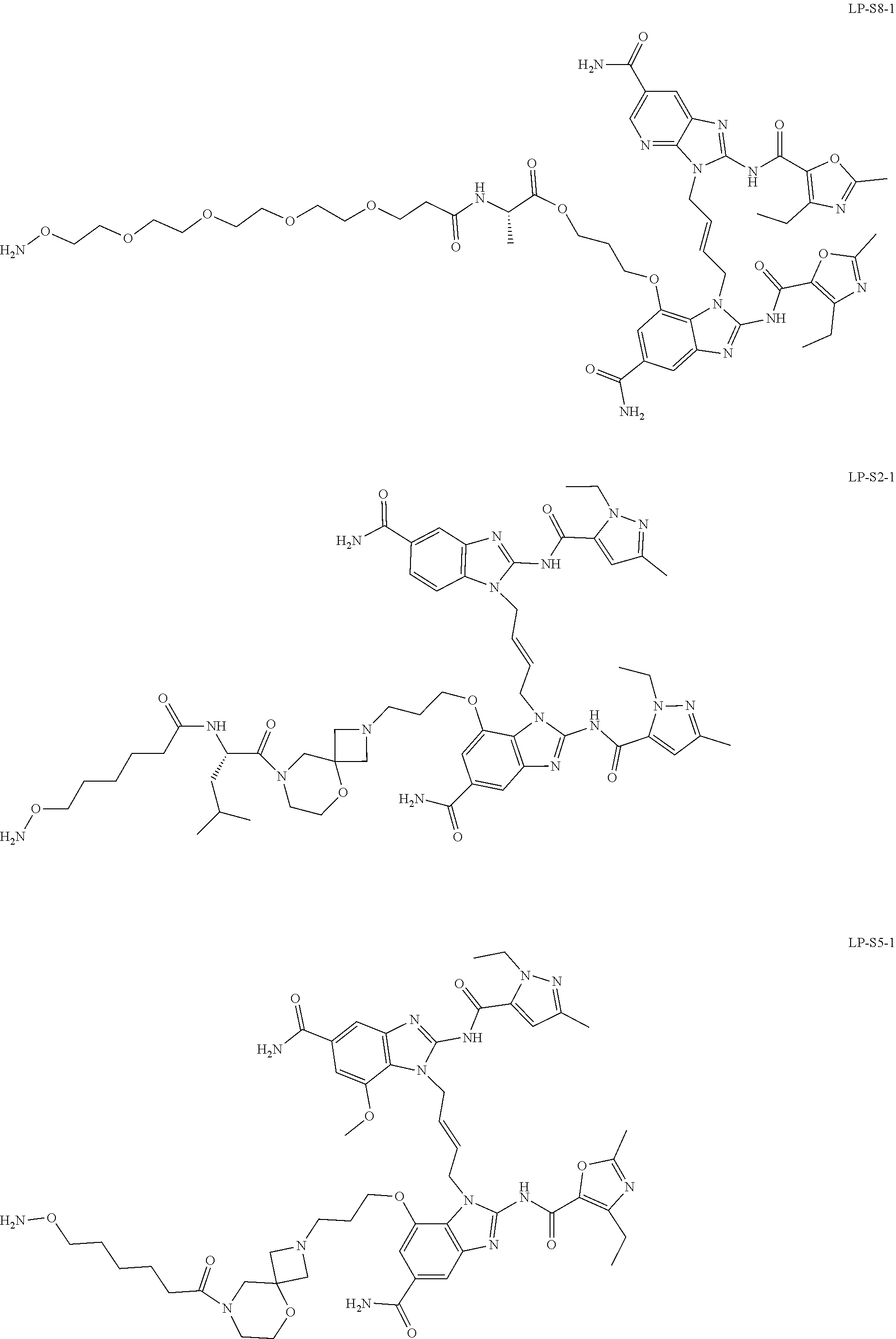 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCOC(=O)[C@H](C)NC(=O)CCOCCOCCOCCOCCON)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)CCCCCON)CCO4)c21 ; CCc1nc(C)oc1C(=O)Nc1nc2cc(C(N)=O)cc(OCCCN3CC4(C3)CN(C(=O)CCCCCON)CCO4)c2n1C/C=C/Cn1c(NC(=O)c2cc(C)nn2CC)nc2cc(C(N)=O)cc(OC)c21]

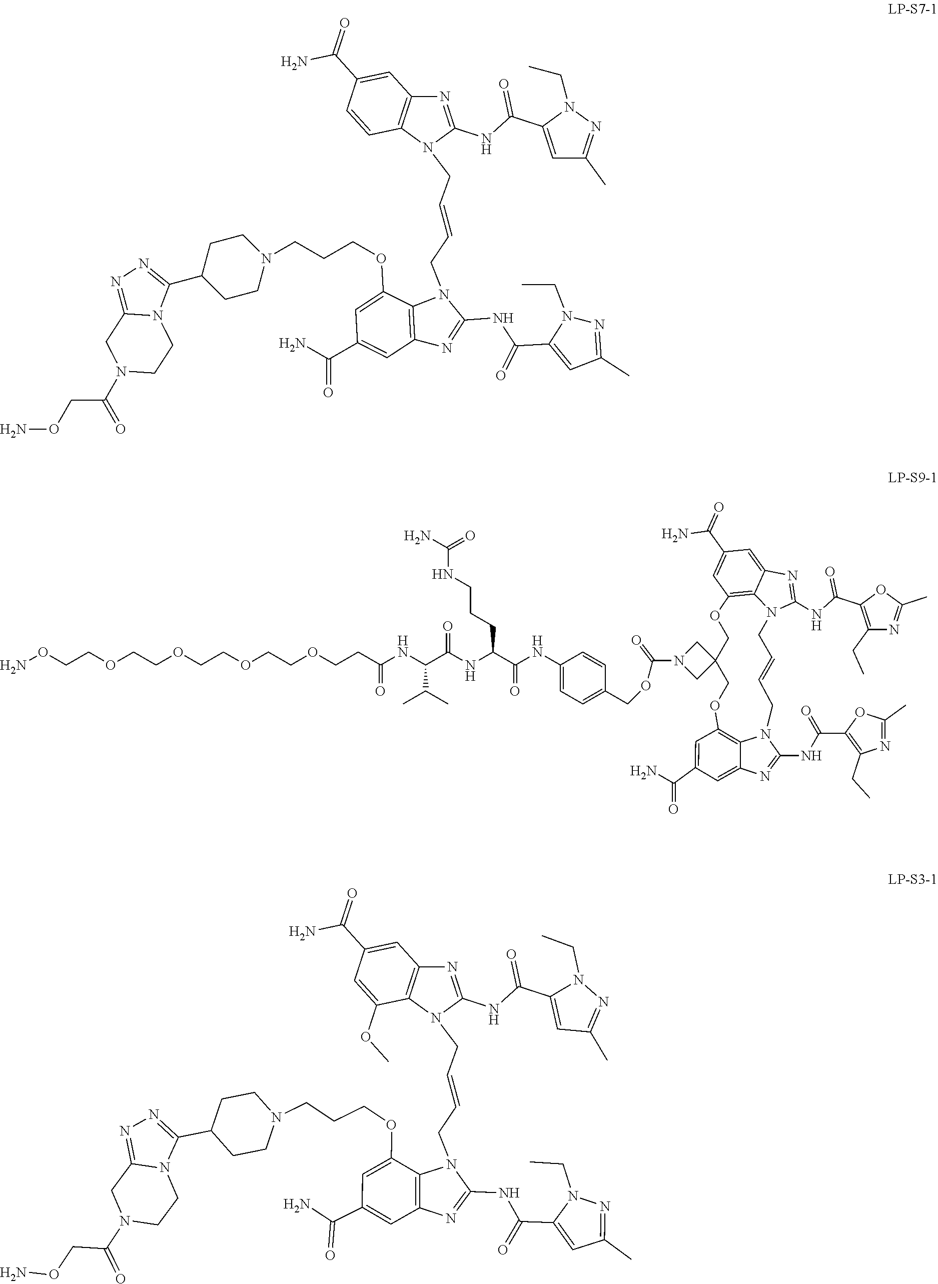 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)CON)C5)CC3)c21 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)[C@H](CCCNC(N)=O)NC(=O)[C@@H](NC(=O)CCOCCOCCOCCOCCON)C(C)C)cc2)C1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

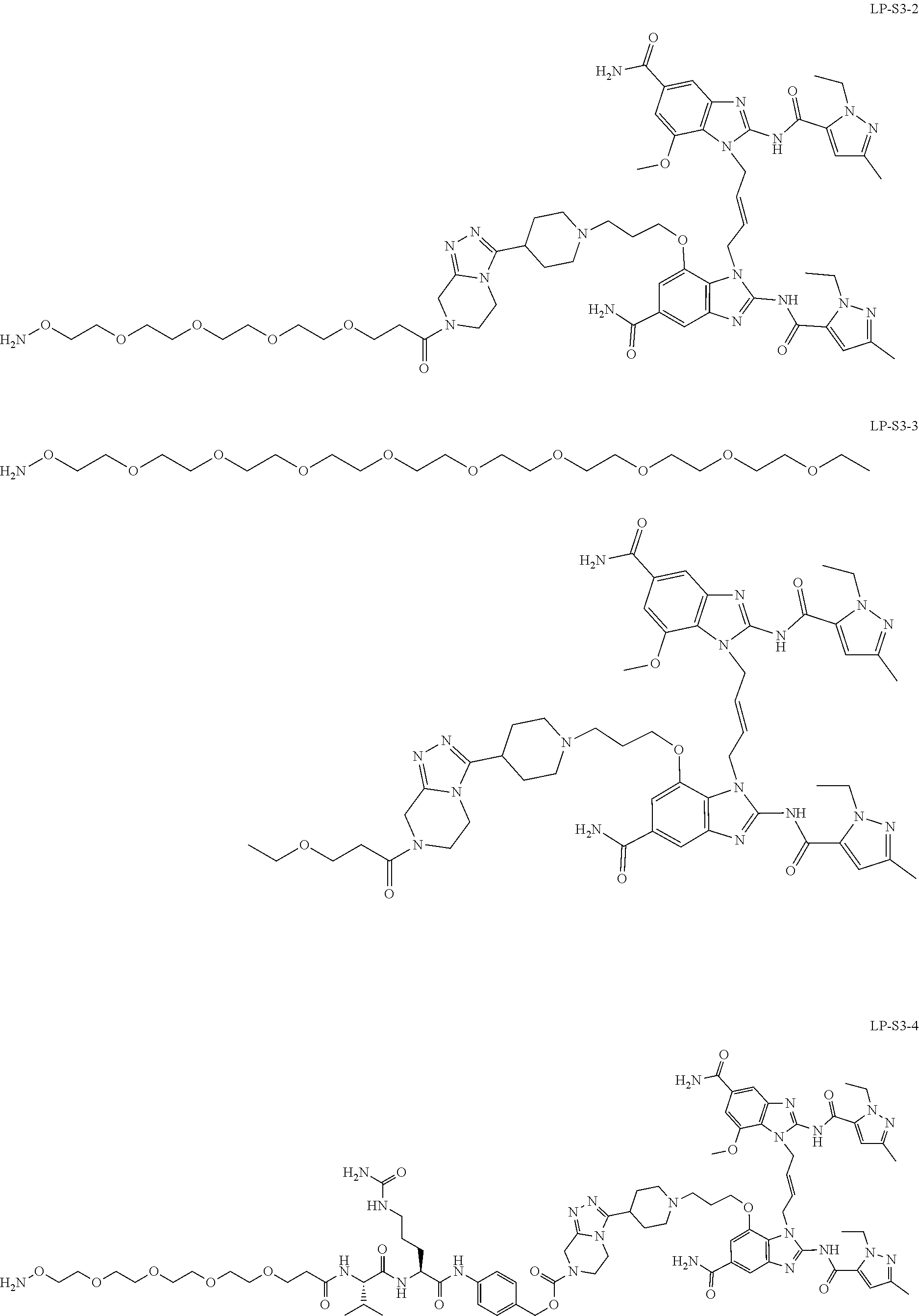

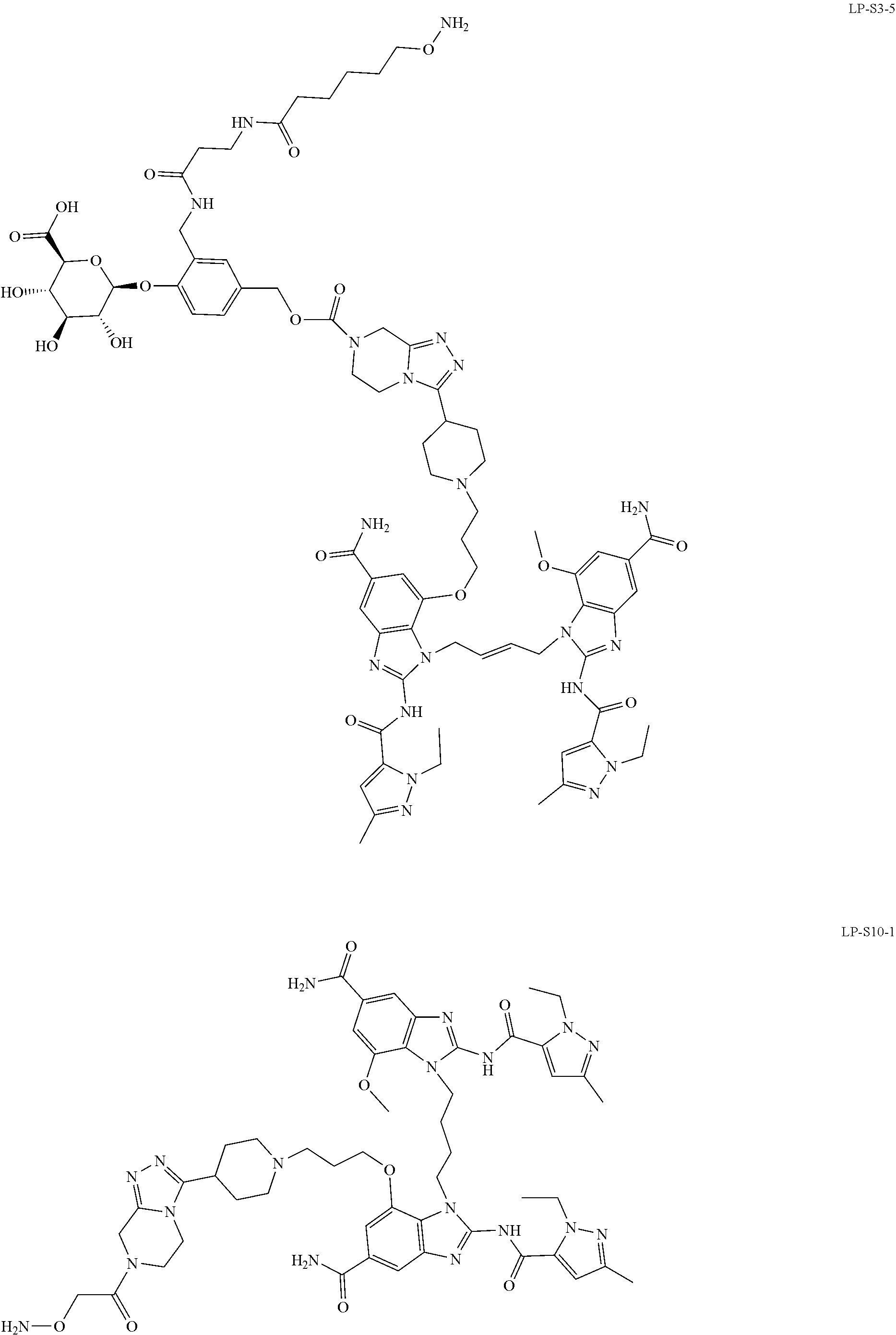 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](O)[C@H](O)[C@H]6O)c(CNC(=O)CCNC(=O)CCCCCON)c4)C5)CC3)c21 ; CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)CON)C5)CC3)c21]

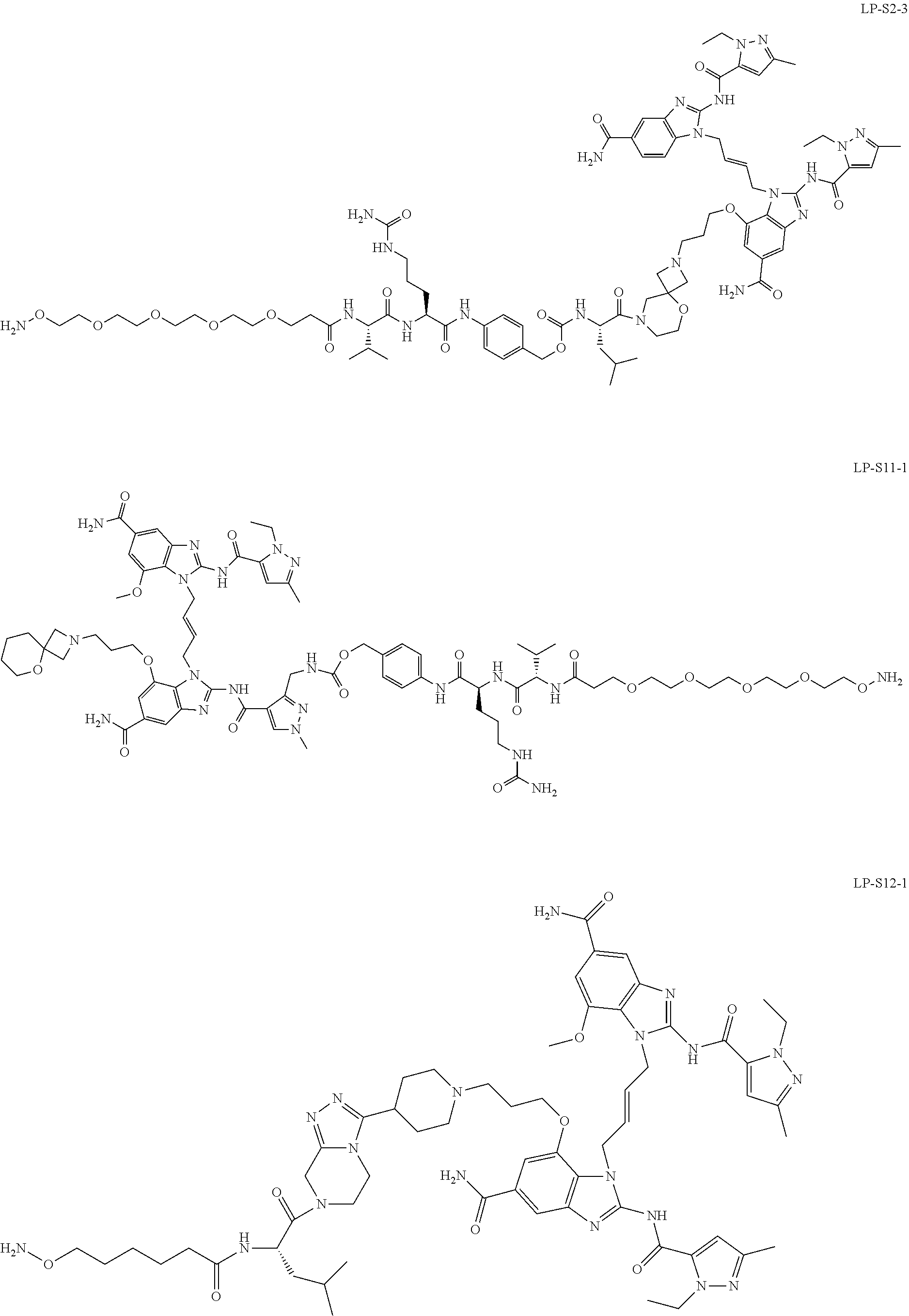

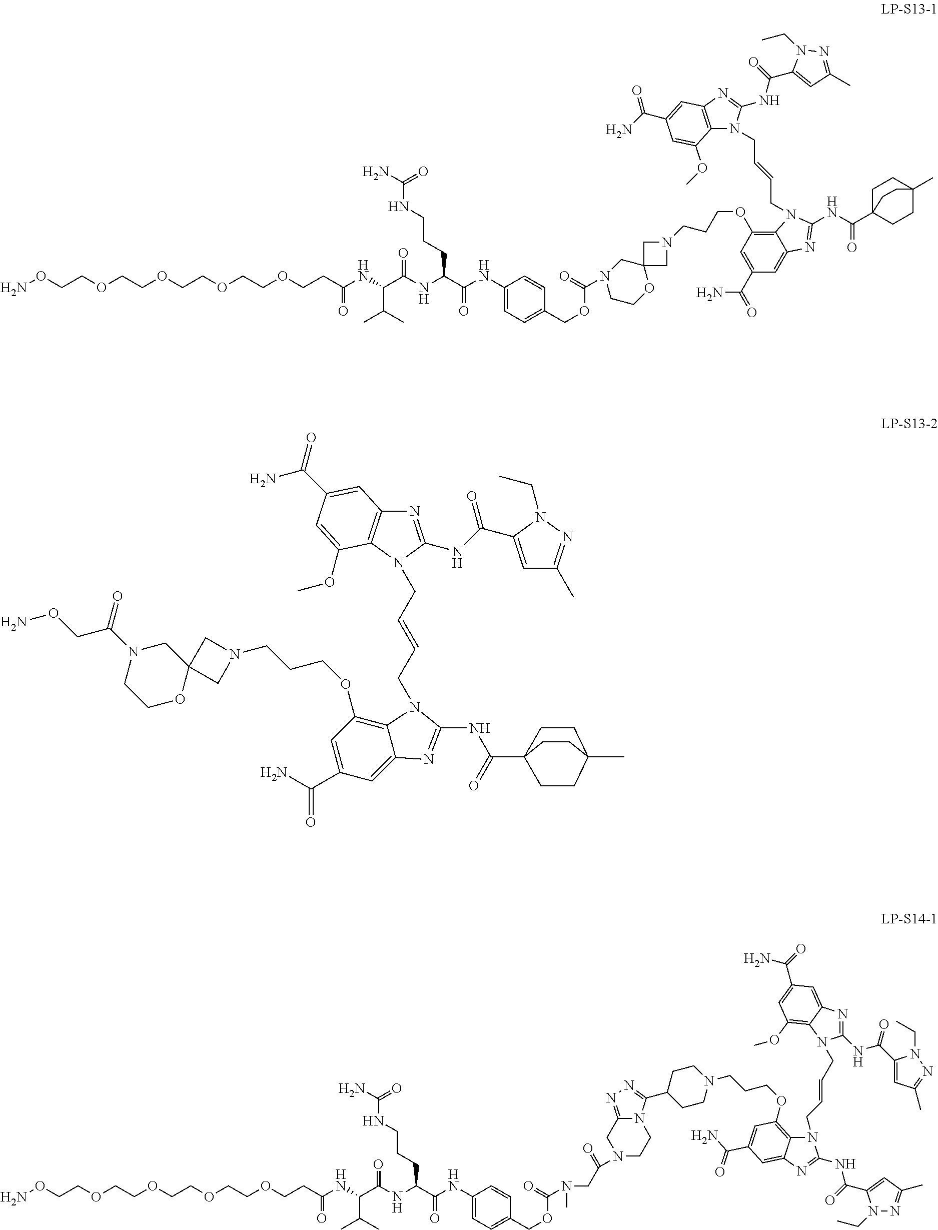

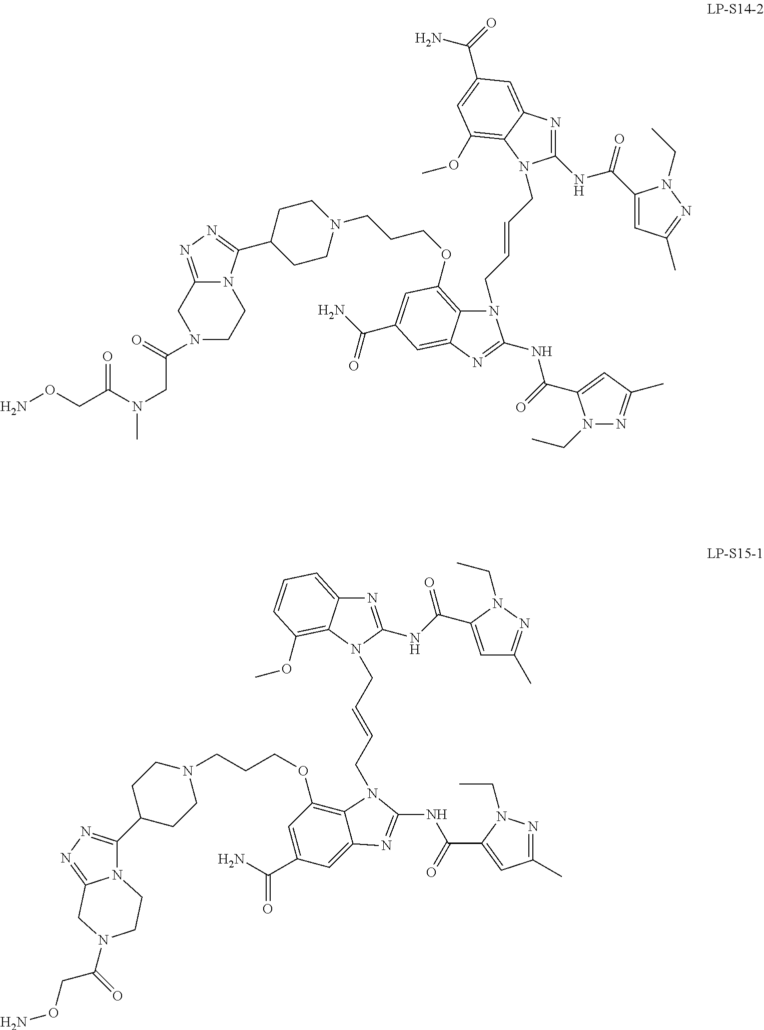 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)CON)C5)CC3)c21 ; CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

Also described herein is a STING agonist residue of the formula:

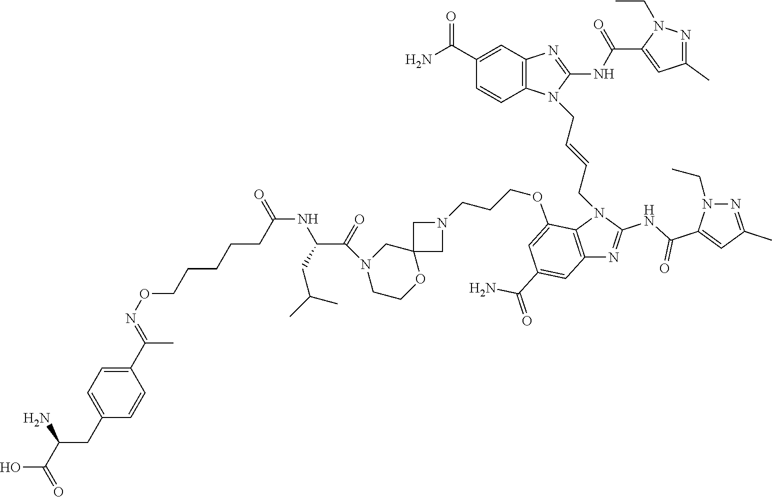

In certain embodiments of Formula (Ia), (Ib), (Ib-1), or (Ib-2),

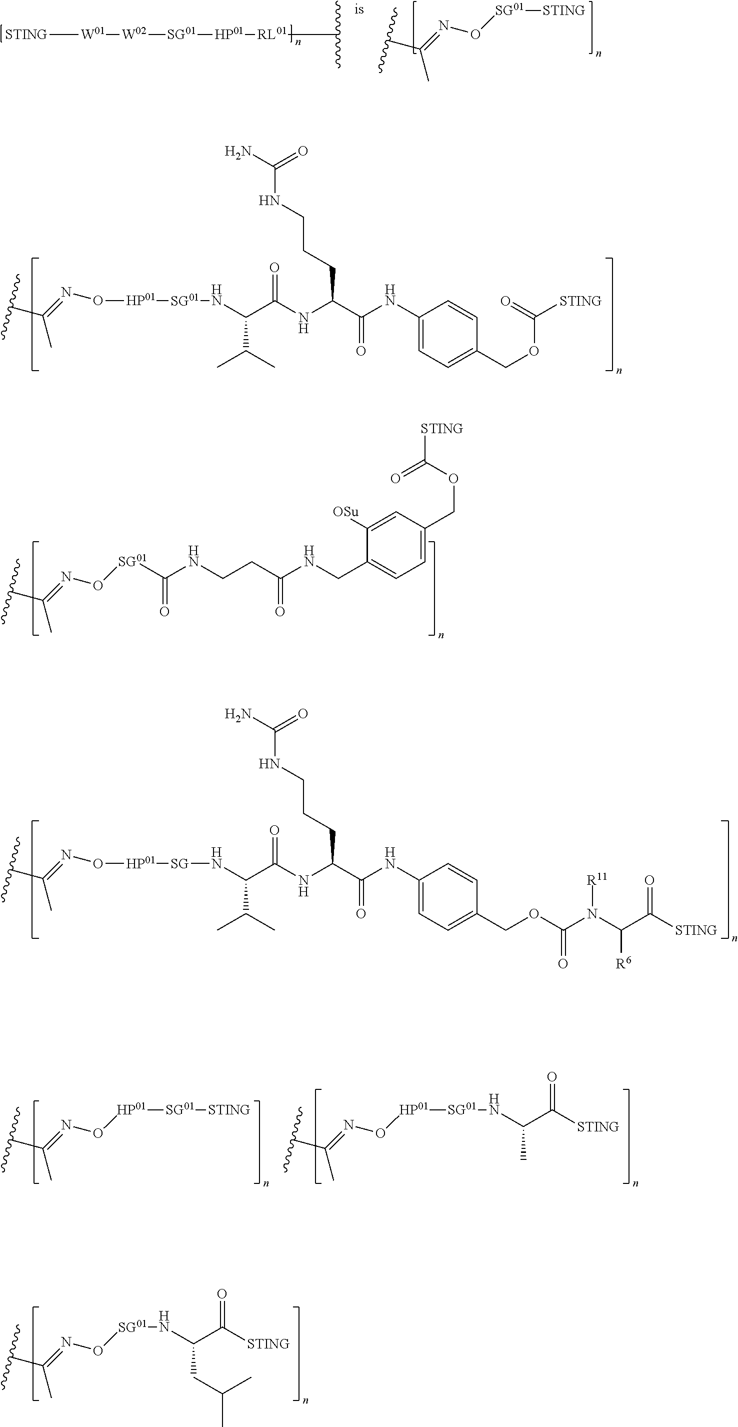

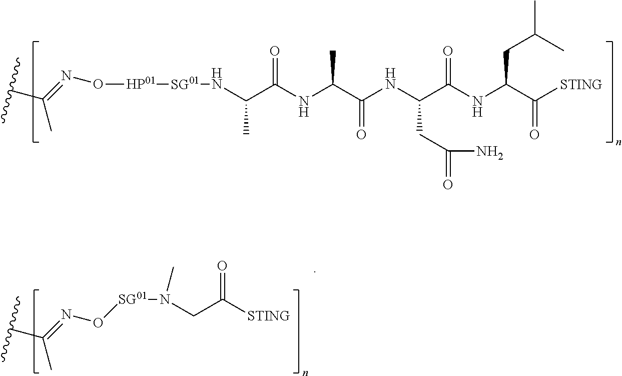

In one embodiment of Formula (I)

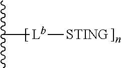

is a structure selected from

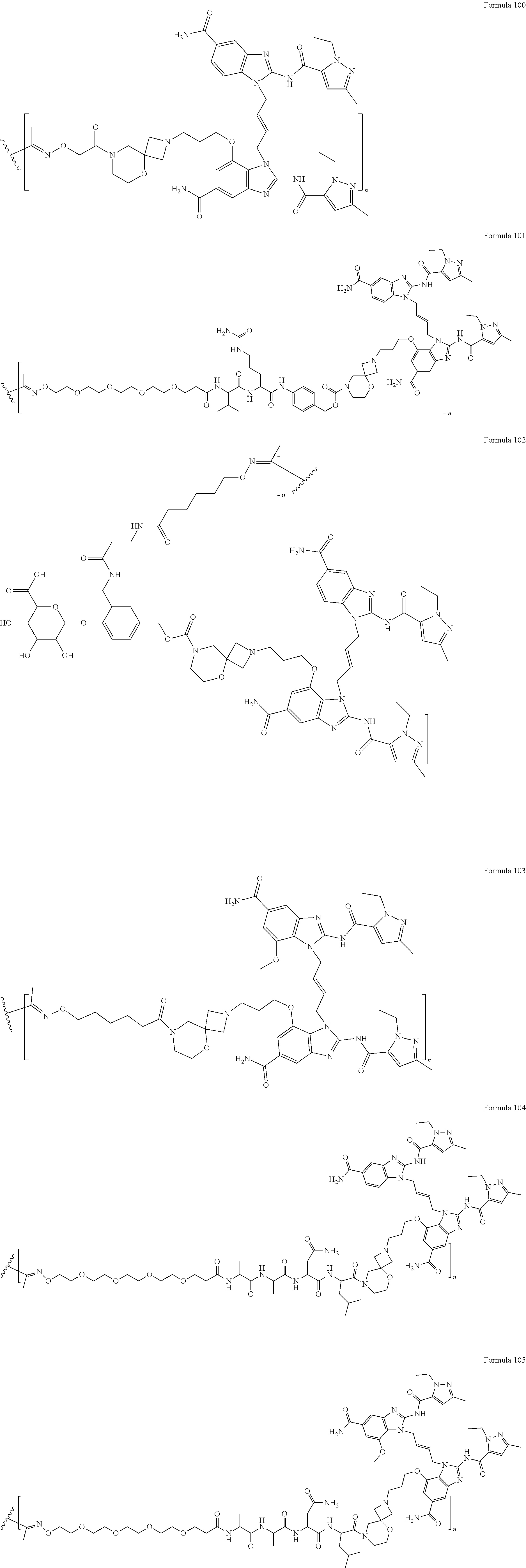

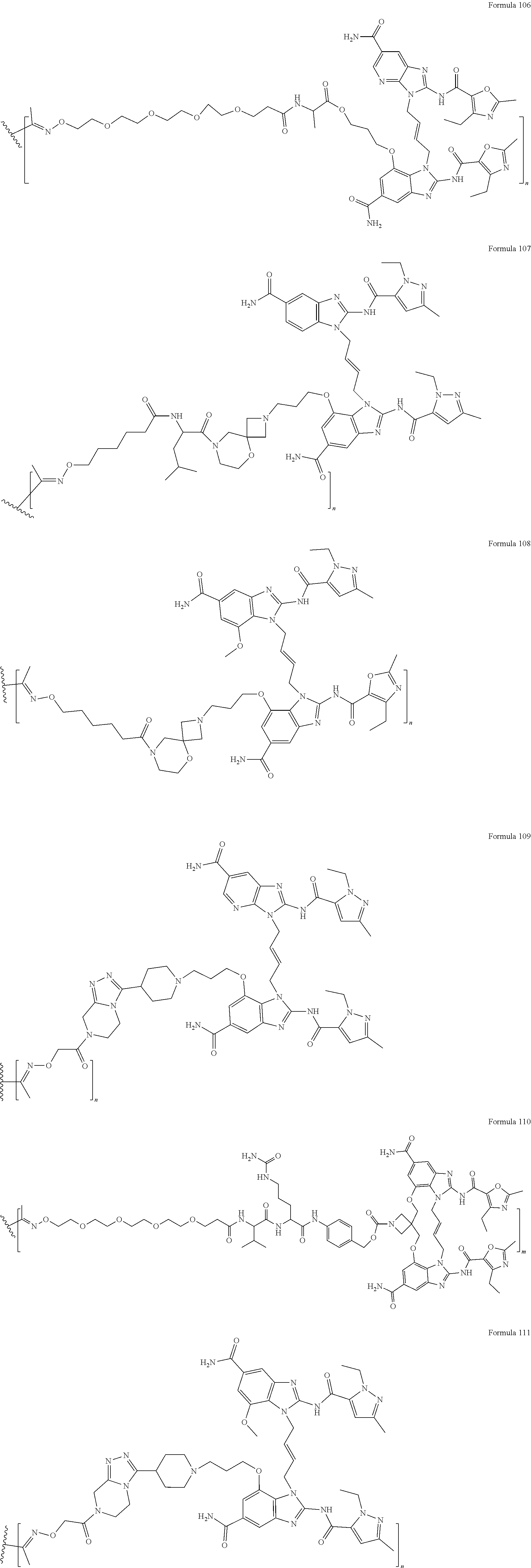

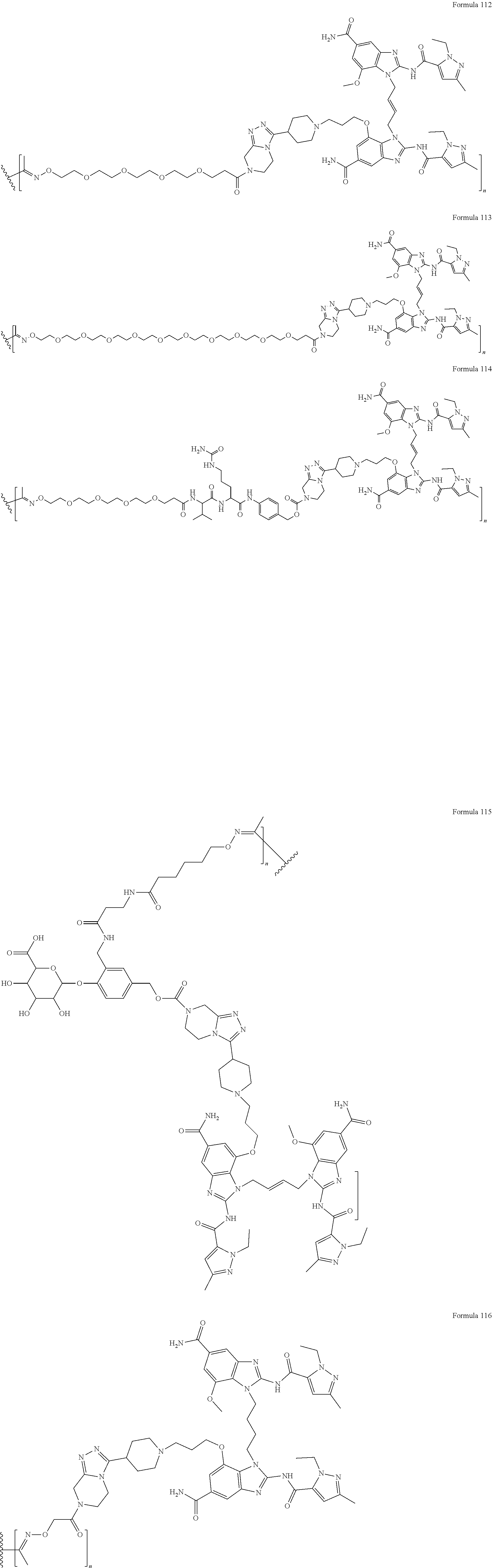

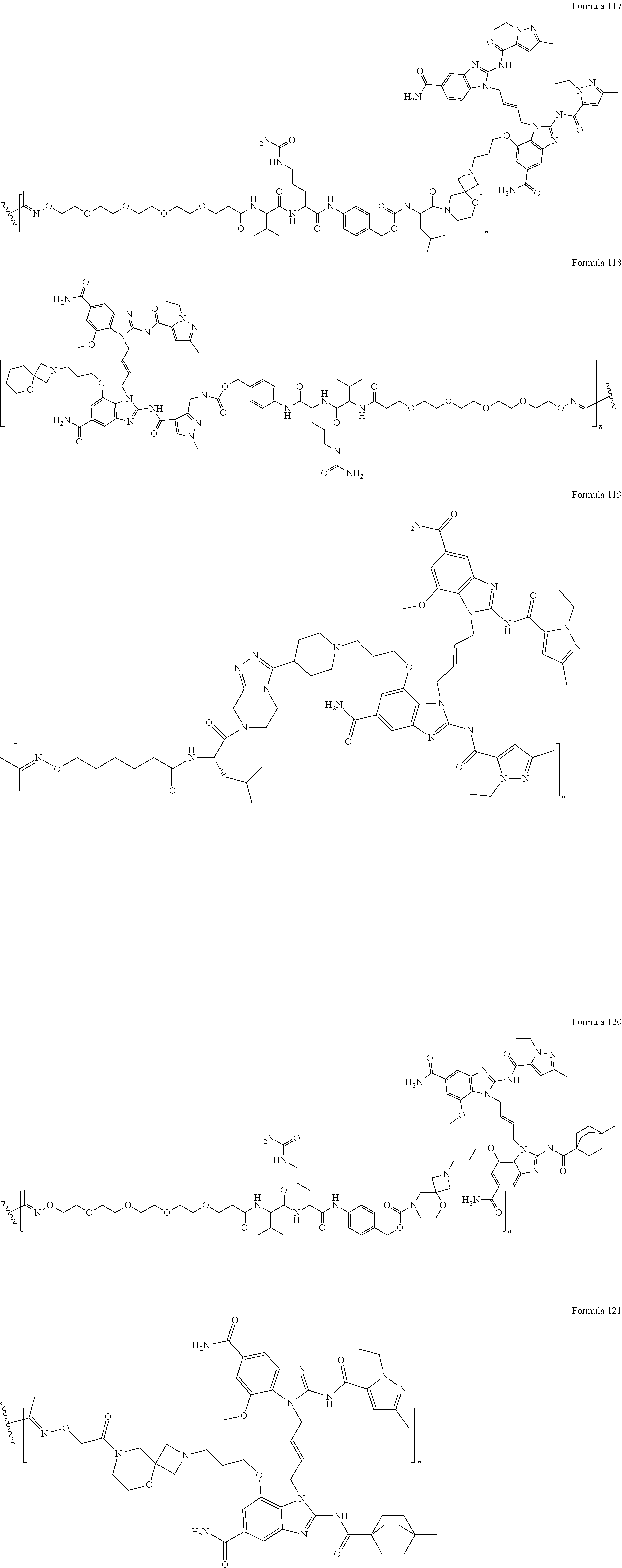

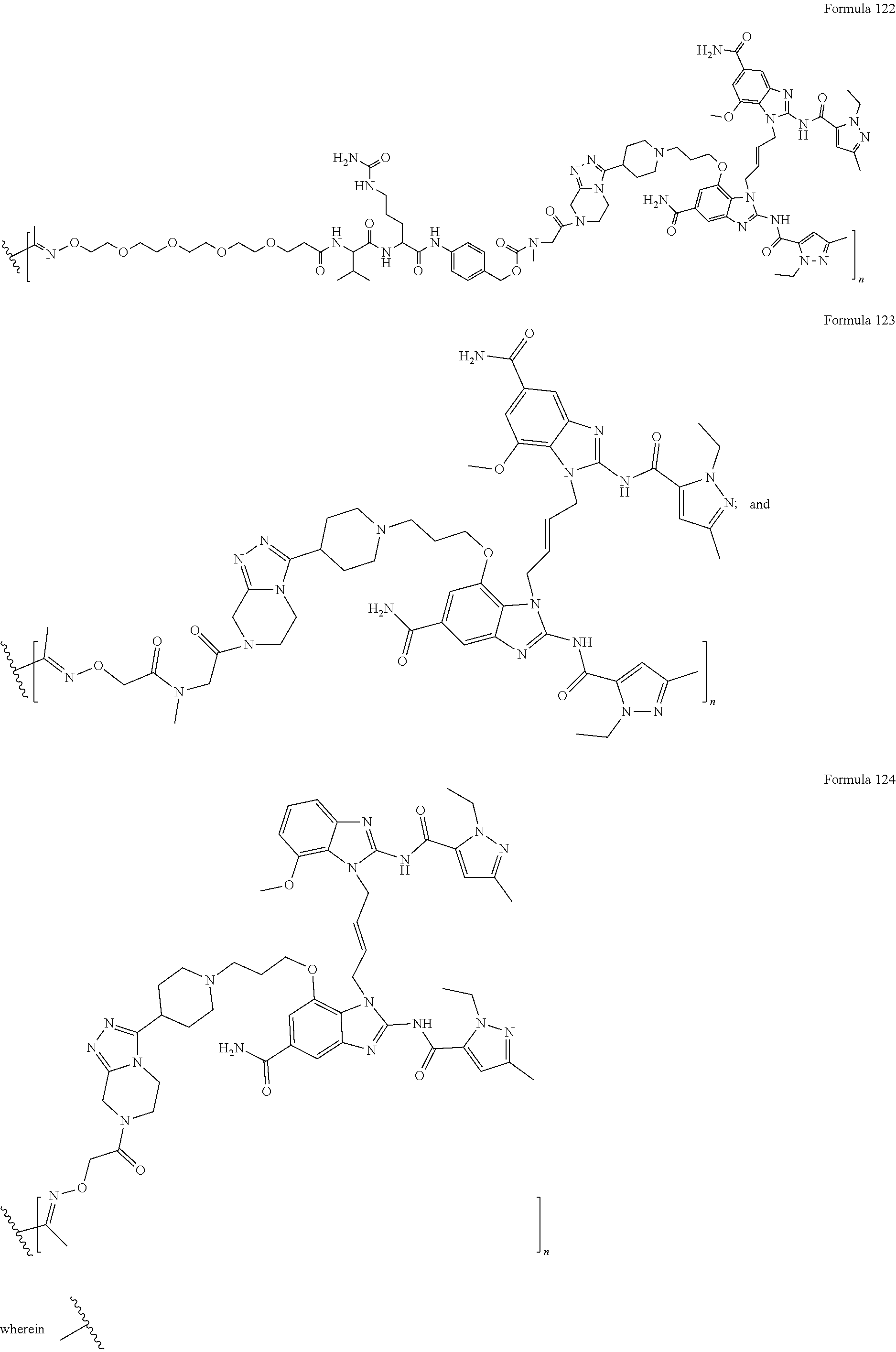

is the point of attachment to the rest of the formula.

In one embodiment of Formula (I),

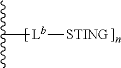

is a structure selected from

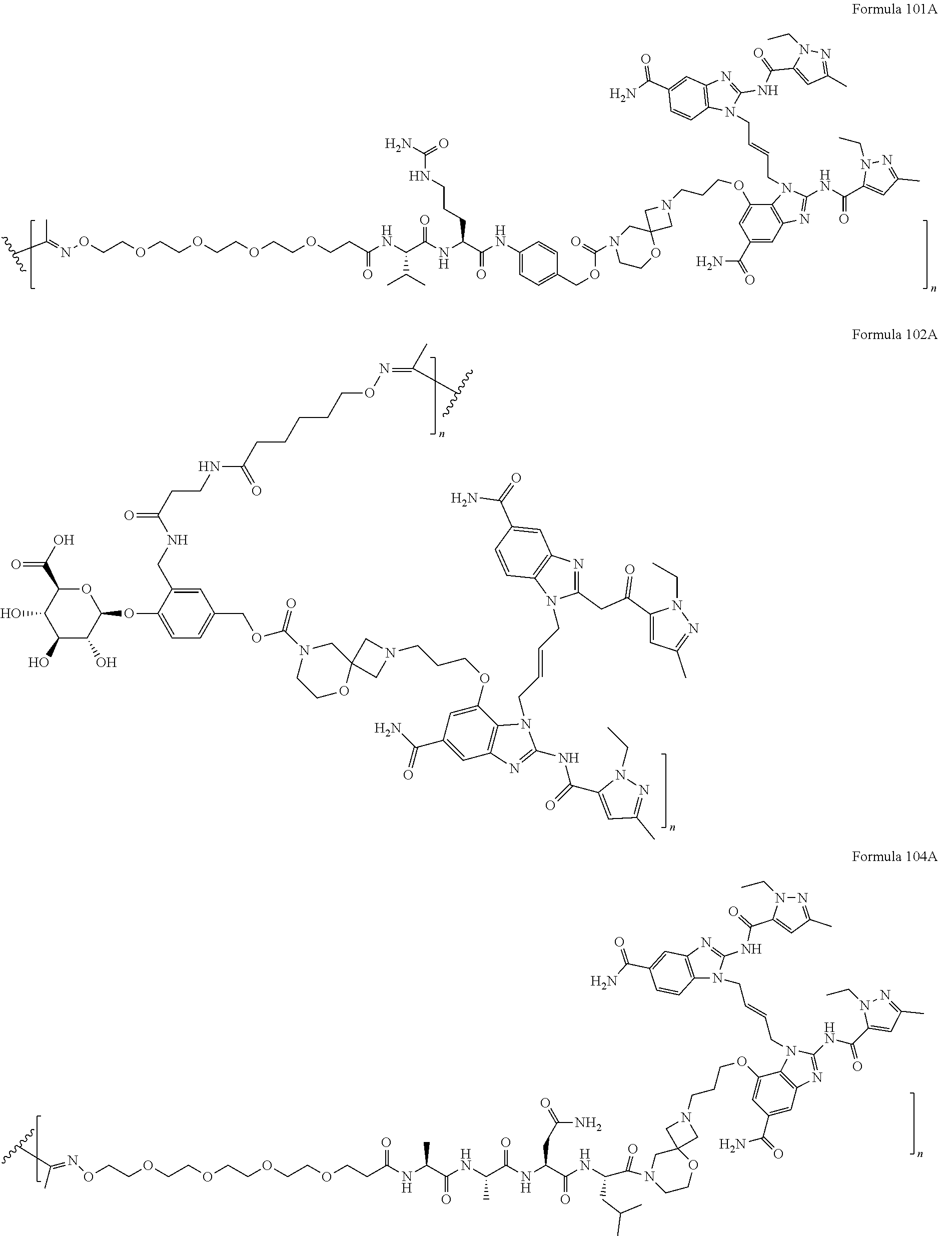

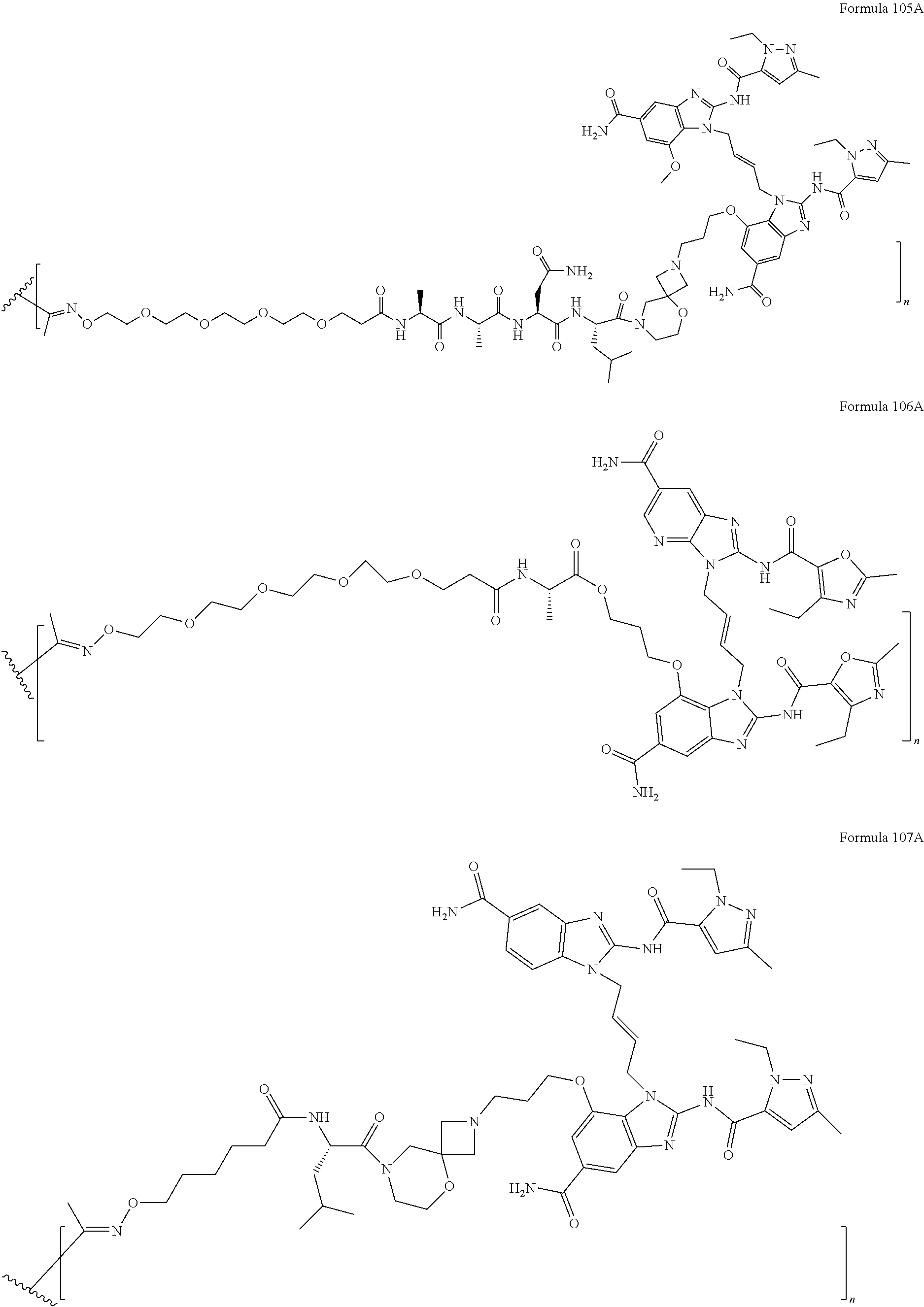

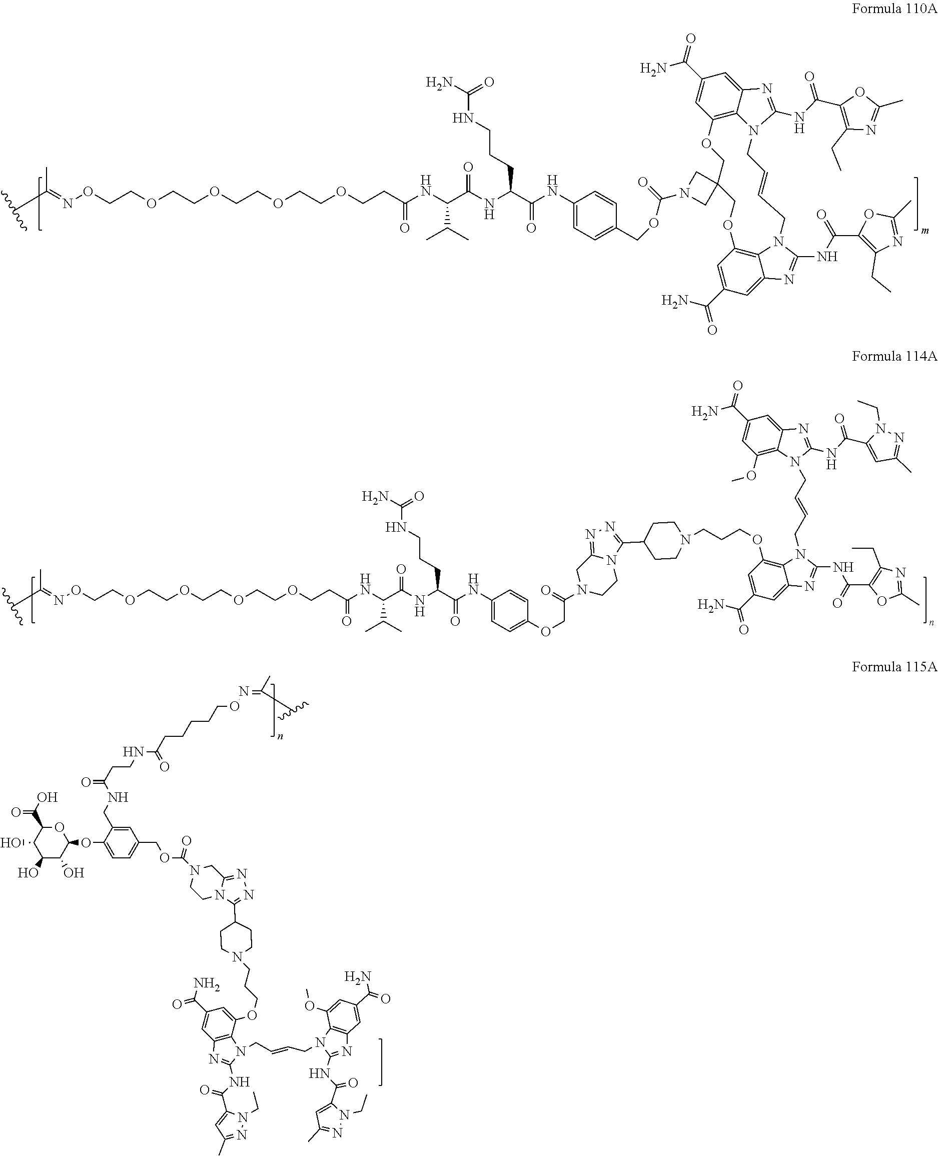

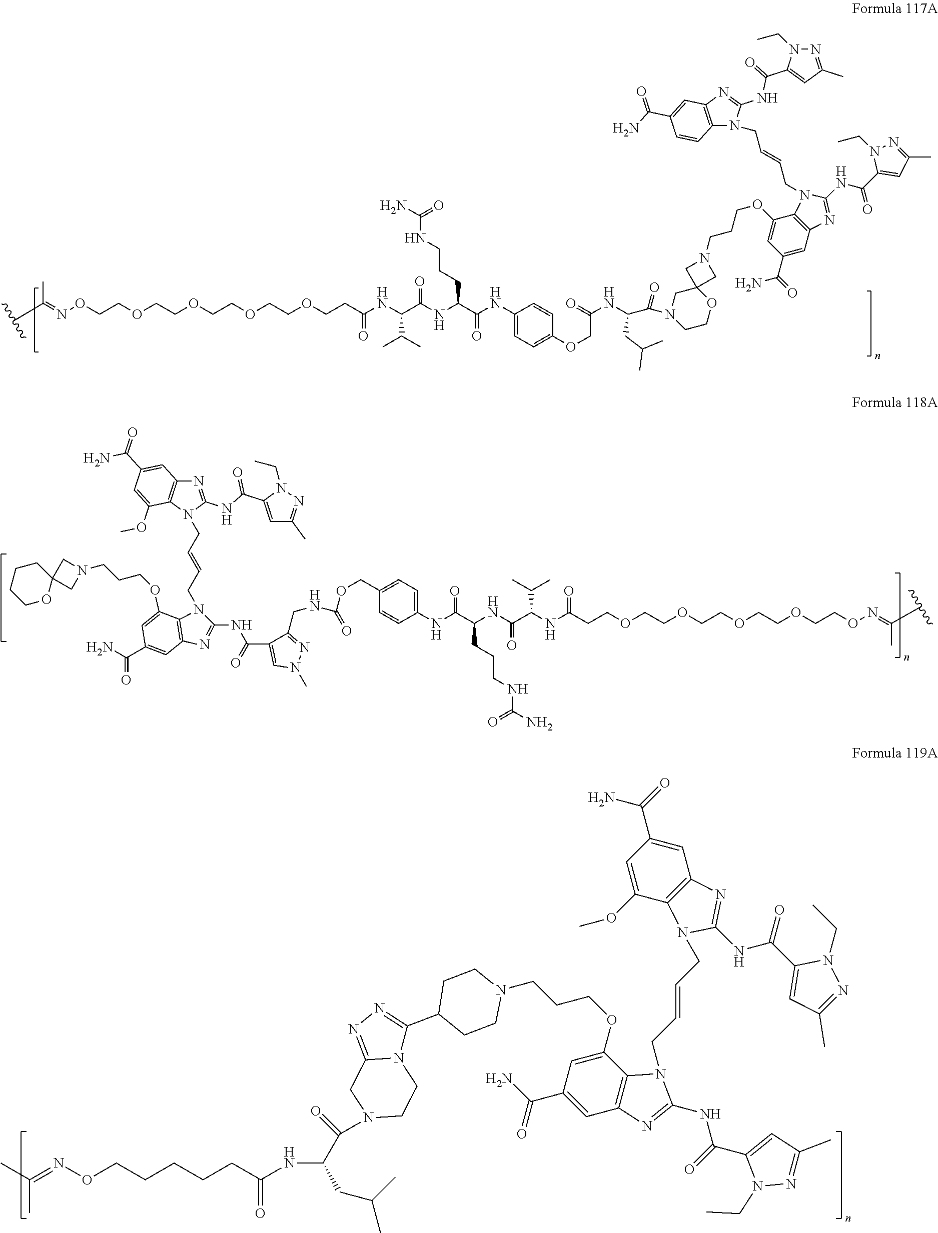

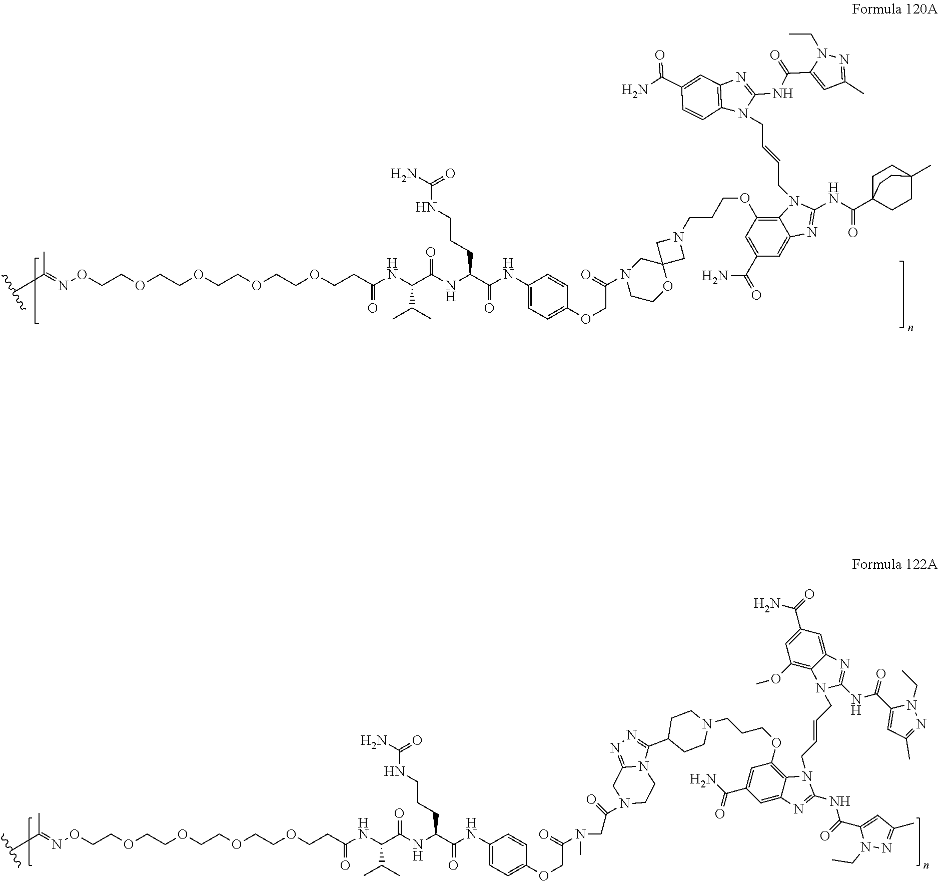

is the point of attachment to the rest of the formula.

In one embodiment of Formula (I),

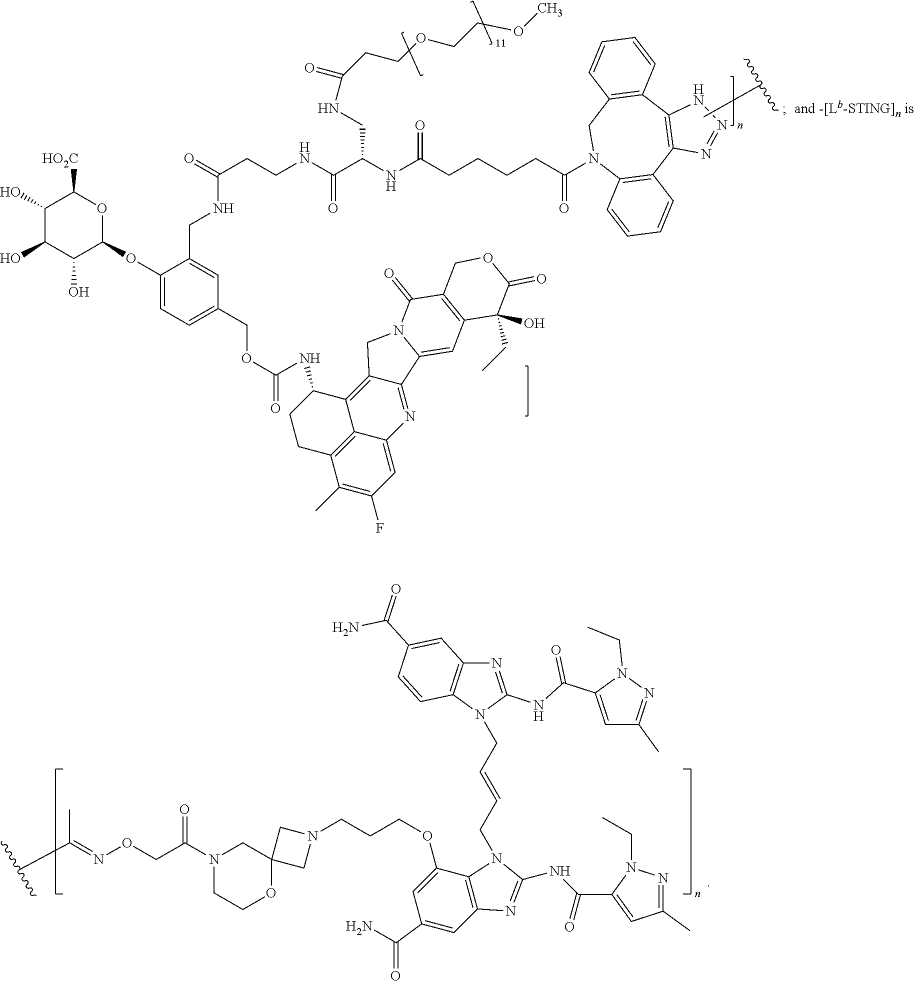

In one embodiment of Formula (I),

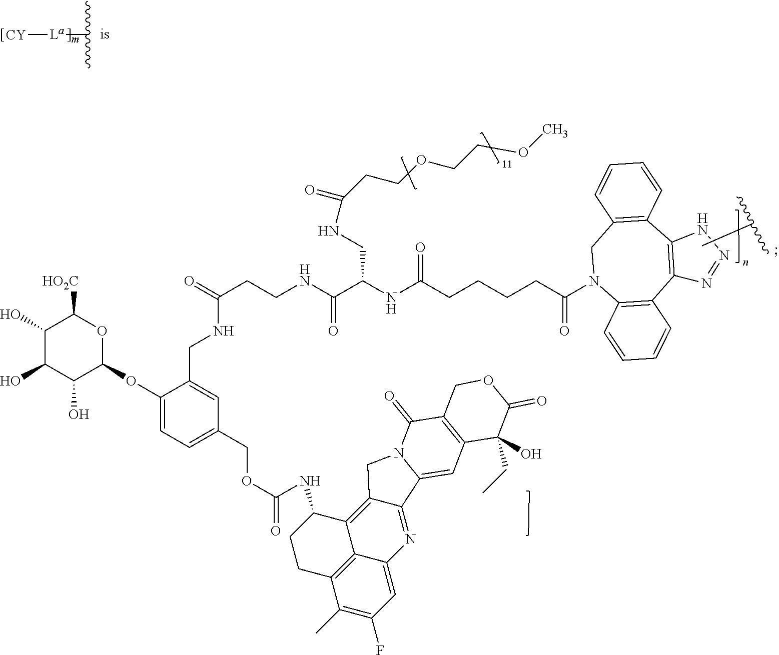

and
-[L^{b}-STRING]ₙ is a structure selected from

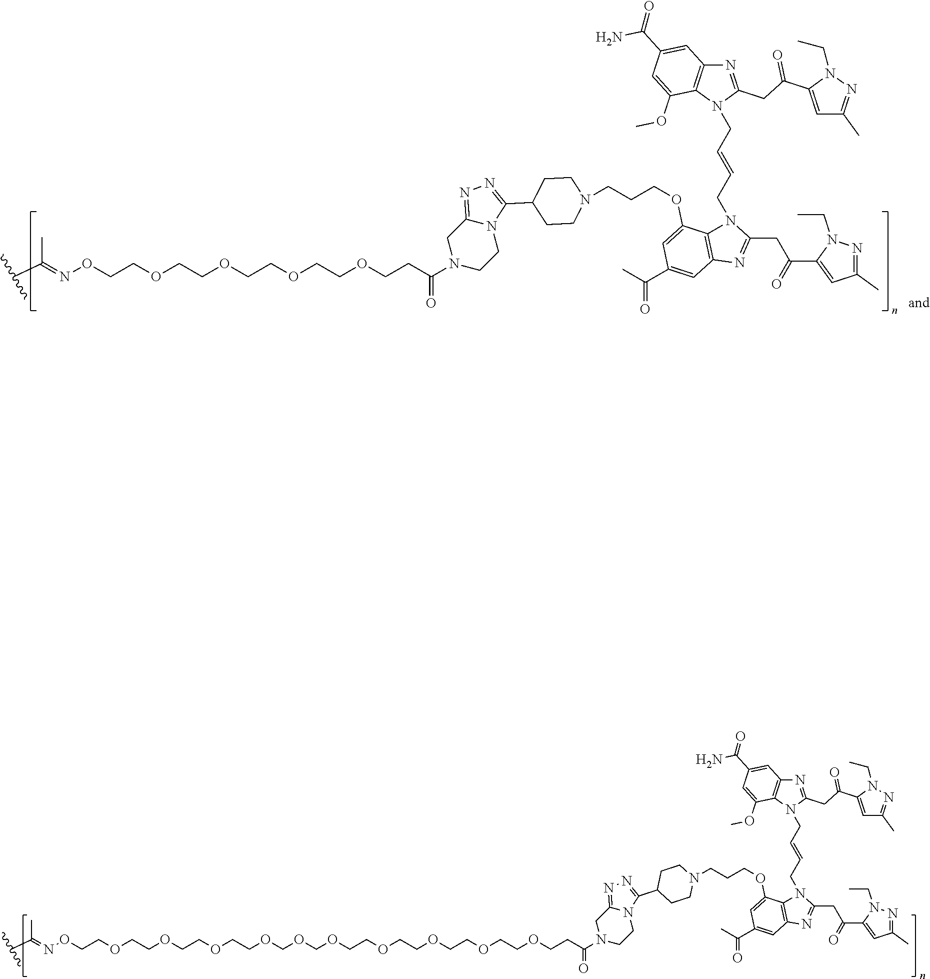

In some embodiments of Formula (I), (Ia), (Ib), (Ic), (Ib-1), (Ib-2), (Ic-1), or (Ic-2) the antibody conjugate comprises Formula 201, Formula 202, or a mixture of Formula 201 and Formula 202. In some embodiments of Formula (I), (Ia), (Ib), (Ic), (Ib-1), (Ib-2), (Ic-1), or (Ic-2) the antibody conjugate comprises Formula 201A, Formula 202A, or a mixture of Formula 201A and Formula 202A. In some embodiments of Formula (I), (Ia), (Ib), (Ic), (Ib-1), (Ib-2), (Ic-1), or (Ic-2) the antibody conjugate comprises Formula 203, Formula 204, or a mixture of Formula 203 and Formula 204. In some embodiments of Formula (I), (Ia), (Ib), (Ic), (Ib-1), (Ib-2), (Ic-1), or (Ic-2) the antibody conjugate comprises Formula 203A, Formula 204A, or a mixture of Formula 203A and Formula 204A. In some embodiments of Formula (I), (Ia), (Ib), (Ic), (Ib-1), (Ib-2), (Ic-1), or (Ic-2) the antibody conjugate comprises Formula 205, Formula 206, or a mixture of Formula 205 and Formula 206. In some embodiments of Formula (I), (Ia), (Ib), (Ic), (Ib-1), (Ib-2), (Ic-1), or (Ic-2) the antibody conjugate comprises Formula 205A, Formula 206A, or a mixture of Formula 205A and Formula 206A. In some embodiments of Formula (I), (Ia), (Ib), (Ic), (Ib-1), (Ib-2), (Ic-1), or (Ic-2) the antibody conjugate comprises Formula 207, Formula 208, or a mixture of Formula 207 and Formula 208. In some embodiments of Formula (I), (Ia), (Ib), (Ic), (Ib-1), (Ib-2), (Ic-1), or (Ic-2) the antibody conjugate comprises Formula 207A, Formula 208A, or a mixture of Formula 207A and Formula 208A. In some embodiments of Formula (I), (Ia), (Ib), (Ic), (Ib-1), (Ib-2), (Ic-1), or (Ic-2) the antibody conjugate comprises Formula 209, Formula 210, or a mixture of Formula 209 and Formula 210. In some embodiments of Formula (I), (Ia), (Ib), (Ic), (Ib-1), (Ib-2), (Ic-1), or (Ic-2) the antibody conjugate comprises Formula 209A, Formula 210A, or a mixture of Formula 209A and Formula 210A.

In some embodiments of Formula (I), (Ia), (Ib), (Ic), (Ib-1), (Ib-2), (Ic-1), or (Ic-2) the antibody conjugate comprises Formula 100. In some embodiments of Formula (I), (Ia), (Ib), (Ic), (Ib-1), (Ib-2), (Ic-1), or (Ic-2) the antibody conjugate comprises Formula 101 or Formula 101A. In some embodiments of Formula (I), (Ia), (Ib), (Ic), (Ib-1), (Ib-2), (Ic-1), or (Ic-2) the antibody conjugate comprises Formula 102 or Formula 102A. In some embodiments of Formula (I), (Ia), (Ib), (Ic), (Ib-1), (Ib-2), (Ic-1), or (Ic-2) the antibody conjugate comprises Formula 103. In some embodiments of Formula (I), (Ia), (Ib), (Ic), (Ib-1), (Ib-2), (Ic-1), or (Ic-2) the antibody conjugate comprises Formula 104 or Formula 104A. In some embodiments of Formula (I), (Ia), (Ib), (Ic), (Ib-1), (Ib-2), (Ic-1), or (Ic-2) the antibody conjugate comprises Formula 105 or Formula 105A. In some embodiments of Formula (I), (Ia), (Ib), (Ic), (Ib-1), (Ib-2), (Ic-1), or (Ic-2) the antibody conjugate comprises Formula 106 or Formula 106A. In some embodiments of Formula (I), (Ia), (Ib), (Ic), (Ib-1), (Ib-2), (Ic-1), or (Ic-2) the antibody conjugate comprises Formula 107 or Formula 107A. In some embodiments of Formula (I), (Ia), (Ib), (Ic), (Ib-1), (Ib-2), (Ic-1), or (Ic-2) the antibody conjugate comprises Formula 108. In some embodiments of Formula (I), (Ia), (Ib), (Ic), (Ib-1), (Ib-2), (Ic-1), or (Ic-2) the antibody conjugate comprises Formula 109. In some embodiments of Formula (I), (Ia), (Ib), (Ic), (Ib-1), (Ib-2), (Ic-1), or (Ic-2) the antibody conjugate comprises Formula 110 or Formula 110A. In some embodiments of Formula (I), (Ia), (Ib), (Ic), (Ib-1), (Ib-2), (Ic-1), or (Ic-2) the antibody conjugate comprises Formula 111. In some embodiments of Formula (I), (Ia), (Ib), (Ic), (Ib-1), (Ib-2), (Ic-1), or (Ic-2) the antibody conjugate comprises Formula 112. In some embodiments of Formula (I), (Ia), (Ib), (Ic), (Ib-1), (Ib-2), (Ic-1), or (Ic-2) the antibody conjugate comprises Formula 113. In some embodiments of Formula (I), (Ia), (Ib), (Ic), (Ib-1), (Ib-2), (Ic-1), or (Ic-2) the antibody conjugate comprises Formula 114 or Formula 114A. In some embodiments of Formula (I), (Ia), (Ib), (Ic), (Ib-1), (Ib-2), (Ic-1), or (Ic-2) the antibody conjugate comprises Formula 115 or Formula 115A. In some embodiments of Formula (I), (Ia), (Ib), (Ic), (Ib-1), (Ib-2), (Ic-1), or (Ic-2) the antibody conjugate comprises Formula 116. In some embodiments of Formula (I), (Ia), (Ib), (Ic), (Ib-1), (Ib-2), (Ic-1), or (Ic-2) the antibody conjugate comprises Formula 117 or Formula 117A. In some embodiments of Formula (I), (Ia), (Ib), (Ic), (Ib-1), (Ib-2), (Ic-1), or (Ic-2) the antibody conjugate comprises Formula 118 or Formula 118A. In some embodiments of Formula (I), (Ia), (Ib), (Ic), (Ib-1), (Ib-2), (Ic-1), or (Ic-2) the antibody conjugate comprises Formula 119 or Formula 119A. In some embodiments of Formula (I), (Ia), (Ib), (Ic), (Ib-1), (Ib-2), (Ic-1), or (Ic-2) the antibody conjugate comprises Formula 120 or Formula 120A. In some embodiments of Formula (I), (Ia), (Ib), (Ic), (Ib-1), (Ib-2), (Ic-1), or (Ic-2) the antibody conjugate comprises Formula 121. In some embodiments of Formula (I), (Ia), (Ib), (Ic), (Ib-1), (Ib-2), (Ic-1), or (Ic-2) the antibody conjugate comprises Formula 122 or Formula 122A. In some embodiments of Formula (I), (Ia), (Ib), (Ic), (Ib-1), (Ib-2), (Ic-1), or (Ic-2) the antibody conjugate comprises Formula 123. In some embodiments of Formula (I), (Ia), (Ib), (Ic), (Ib-1), (Ib-2), (Ic-1), or (Ic-2) the antibody conjugate comprises Formula 124.

In some embodiments of Formula (I), (Ia), (Ib), (Ic), (Ib-1), (Ib-2), (Ic-1), or (Ic-2) the antibody conjugate comprises Formula 201, Formula 202, Formula 203, Formula 204, Formula 205, Formula 206, Formula 207, Formula 208, Formula 209, or Formula 210, wherein the structure binds to para-azidomethyl-L-phenylalanine (pAMF) at one or more sites.

In some embodiments of Formula (I), (Ia), (Ib), (Ic), (Ib-1), (Ib-2), (Ic-1), or (Ic-2) the antibody conjugate comprises Formula 201A, Formula 202A, Formula 203A, Formula 204A, Formula 205A, Formula 206A, Formula 207A, Formula 208A, Formula 209A, or Formula 210A, wherein the structure binds to para-azidomethyl-L-phenylalanine (pAMF) at one or more sites.

In some embodiments of Formula (I), (Ia), (Ib), (Ic), (Ib-1), (Ib-2), (Ic-1), or (Ic-2) the antibody conjugate comprises Formula 201, Formula 202, Formula 203, Formula 204, Formula 205, Formula 206, Formula 207, Formula 208, Formula 209, or Formula 210, wherein the structure binds to para-azidomethyl-L-phenylalanine (pAMF) at HC-F241 and HC-404. In some embodiments of Formula (I), (Ia), (Ib), (Ic), (Ib-1), (Ib-2), (Ic-1), or (Ic-2) the antibody conjugate comprises Formula 201, Formula 202, Formula 203, Formula 204, Formula 205, Formula 206, Formula 207, Formula 208, Formula 209, or Formula 210, wherein the structure binds to para-azidomethyl-L-phenylalanine (pAMF) at HC-Y180 and HC-404.

In some embodiments of Formula (I), (Ia), (Ib), (Ic), (Ib-1), (Ib-2), (Ic-1), or (Ic-2) the antibody conjugate comprises Formula 201A, Formula 202A, Formula 203A, Formula 204A, Formula 205A, Formula 206A, Formula 207A, Formula 208A, Formula 209A, or Formula 210A, wherein the structure binds to para-azidomethyl-L-phenylalanine (pAMF) at HC-F241 and HC-404. In some embodiments of Formula (I), (Ia), (Ib), (Ic), (Ib-1), (Ib-2), (Ic-1), or (Ic-2) the antibody conjugate comprises Formula 201A, Formula 202A, Formula 203A, Formula 204A, Formula 205A, Formula 206A, Formula 207A, Formula 208A, Formula 209A, or Formula 210A, wherein the structure binds to para-azidomethyl-L-phenylalanine (pAMF) at HC-Y180 and HC-404.

In some embodiments of Formula (I), (Ia), (Ib), (Ic), (Ib-1), (Ib-2), (Ic-1), or (Ic-2) the antibody conjugate comprises Formula 201, Formula 202, or a mixture thereof, wherein the structure binds to para-azidomethyl-L-phenylalanine (pAMF) at HC-F241 and HC-404. In some embodiments of Formula (I), (Ia), (Ib), (Ic), (Ib-1), (Ib-2), (Ic-1), or (Ic-2) the antibody conjugate comprises Formula 201, Formula 202, or a mixture thereof, wherein the structure binds to para-azidomethyl-L-phenylalanine (pAMF) at HC-Y180 and HC-404.

In some embodiments of Formula (I), (Ia), (Ib), (Ic), (Ib-1), (Ib-2), (Ic-1), or (Ic-2) the antibody conjugate comprises Formula 201A, Formula 202A, or a mixture thereof, wherein the structure binds to para-azidomethyl-L-phenylalanine (pAMF) at HC-F241 and HC-404. In some embodiments of Formula (I), (Ia), (Ib), (Ic), (Ib-1), (Ib-2), (Ic-1), or (Ic-2) the antibody conjugate comprises Formula 201A, Formula 202A, or a mixture thereof, wherein the structure binds to para-azidomethyl-L-phenylalanine (pAMF) at HC-Y180 and HC-404.

In some embodiments of Formula (I), (Ia), (Ib), (Ic), (Ib-1), (Ib-2), (Ic-1), or (Ic-2) the antibody conjugate comprises Formula 203, Formula 204, or a mixture thereof, wherein the structure binds to para-azidomethyl-L-phenylalanine (pAMF) at HC-F241 and HC-404. In some embodiments of Formula (I), (Ia), (Ib), (Ic), (Ib-1), (Ib-2), (Ic-1), or (Ic-2) the antibody conjugate comprises Formula 203, Formula 204, or a mixture thereof, wherein the structure binds to para-azidomethyl-L-phenylalanine (pAMF) at HC-Y180 and HC-404.

In some embodiments of Formula (I), (Ia), (Ib), (Ic), (Ib-1), (Ib-2), (Ic-1), or (Ic-2) the antibody conjugate comprises Formula 203A, Formula 204A, or a mixture thereof, wherein the structure binds to para-azidomethyl-L-phenylalanine (pAMF) at HC-F241 and HC-404. In some embodiments of Formula (I), (Ia), (Ib), (Ic), (Ib-1), (Ib-2), (Ic-1), or (Ic-2) the antibody conjugate comprises Formula 203A, Formula 204A, or a mixture thereof, wherein the structure binds to para-azidomethyl-L-phenylalanine (pAMF) at HC-Y180 and HC-404.

In some embodiments of Formula (I), (Ia), (Ib), (Ic), (Ib-1), (Ib-2), (Ic-1), or (Ic-2) the antibody conjugate comprises a formula selected from Formula 100-Formula 124 wherein the structure binds to para-acetyl-L-phenylalanine (pAcF) at one or more sites. In some embodiments of Formula (I), (Ia), (Ib), (Ic), (Ib-1), (Ib-2), (Ic-1), or (Ic-2) the antibody conjugate comprises a formula selected from Formula 100-Formula 124 wherein the structure binds to para-acetyl-L-phenylalanine (pAcF) at LC-K42.

In some embodiments of Formula (I), (Ia), (Ib), (Ic), (Ib-1), (Ib-2), (Ic-1), or (Ic-2) the antibody conjugate comprises (a) Formula 201, Formula 202, or a mixture thereof, wherein the structure binds to para-azidomethyl-L-phenylalanine (pAMF) at HC-F241 and HC-404 and (b) a formula selected from Formula 100-Formula 124 wherein the structure binds to para-acetyl-L-phenylalanine (pAcF) at LC-K42.

In some embodiments of Formula (I), (Ia), (Ib), (Ic), (Ib-1), (Ib-2), (Ic-1), or (Ic-2) the antibody conjugate comprises (a) Formula 201, Formula 202, or a mixture thereof, wherein the structure binds to para-azidomethyl-L-phenylalanine (pAMF) at HC-Y180 and HC-404 and (b) a formula selected from Formula 100-Formula 124 wherein the structure binds to para-acetyl-L-phenylalanine (pAcF) at LC-K42.

In some embodiments of Formula (I), (Ia), (Ib), (Ic), (Ib-1), (Ib-2), (Ic-1), or (Ic-2) the antibody conjugate comprises (a) Formula 203, Formula 204, or a mixture thereof, wherein the structure binds to para-azidomethyl-L-phenylalanine (pAMF) at HC-F241 and HC-404 and (b) a formula selected from Formula 100-Formula 124 wherein the structure binds to para-acetyl-L-phenylalanine (pAcF) at LC-K42.

In some embodiments of Formula (I), (Ia), (Ib), (Ic), (Ib-1), (Ib-2), (Ic-1), or (Ic-2) the antibody conjugate comprises (a) Formula 203, Formula 204, or a mixture thereof, wherein the structure binds to para-azidomethyl-L-phenylalanine (pAMF) at HC-Y180 and HC-404 and (b) a formula selected from Formula 100-Formula 124 wherein the structure binds to para-acetyl-L-phenylalanine (pAcF) at LC-K42.

### 3. Anti-TROP2 Antibody or Antigen Binding Fragment Thereof (TROP2)

The conjugates described herein comprise an anti-TROP2 antibody or antigen binding fragment thereof. The anti-TROP2 antibody or antigen binding fragment thereof selectively binds TROP2, has a property capable of recognizing a TROP2 expressing cell (for example, a tumor cell), has a property of internalizing in a TROP2 expressing cell. In some embodiments, the conjugates described herein comprise means for binding TROP2.

In some embodiments, the antibody or antigen binding fragment thereof selectively binds to human TROP2. In some embodiments, the antibody or antigen binding fragment thereof selectively binds to the extracellular domain of human TROP2.

In some embodiments, the antibody or antigen binding fragment thereof binds to a homolog of human TROP2 from a species selected from a group consisting of monkeys, mice, dogs, cats, rats, cows, horses, goats and sheep. In some embodiments, the homolog is a cynomolgus monkey TROP2, a mouse TROP2 or a murine TROP2.

In some embodiments, the antibody comprises a heavy chain. In some embodiments, the heavy chain is selected from the group consisting of IgA, IgD, IgE, IgG or IgM. In some embodiments, the heavy chain is an IgG. In some embodiments, the heavy chain is an IgG1. In some embodiments, the heavy chain is an IgG2. In some embodiments, the heavy chain is an IgG3. In some embodiments, the heavy chain is an IgG4.

In some embodiments, the antibody or antigen binding fragment thereof comprises a light chain. In some embodiments, the light chain is a kappa light chain. In some embodiments, the light chain is a lambda light chain.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some embodiments, the antibody is an affinity matured antibody derived from an illustrative sequence provided in this disclosure.

In some embodiments, the antibody is an antibody fragment. In some embodiments, the antibody fragment is an Fv fragment. In some embodiments, the antibody fragment is a Fab fragment. In some embodiments, the antibody fragment is a F(ab′)₂ fragment. In some embodiments, the antibody fragment is a Fab′ fragment. In some embodiments, the antibody fragment is an scFv (sFv) fragment. In some embodiments, the antibody fragment is an scFv-Fc fragment.

The anti TROP2 antibody can be any anti-TROP2 antibody, or an antigen binding fragment thereof.

In certain embodiments, the anti-TROP2 antibodies that can be used for this invention are not limited but selected from one of the antibodies that are incorporated in the following ADCs; GS-0132, IMMU-132, Dato-DXd, DS-1062a, MK-2870, SKB264, SHR-A1921, 9MW2921, BAT8008, BIO-106, BL-M02D1, DB-1305, ESG-401, HS-20105, IBI130, LCB84, MHB036C, BAT8003, DXC1002, FDA018, FZ-AD004, JS108, PF-06664178 or STI-3258.

In certain embodiments, the anti-TROP2 antibody is selected from Sacituzumab (hRS7) and Datopotamab.

In some embodiments, the antibody comprises three CDRs of a heavy chain selected from the group consisting of SEQ ID NOS:12, 13, 15, and 16, and three CDRs of a light chain selected from the group consisting of SEQ ID NOS:14, and 17. In some embodiments, the antibody comprises three CDRs of a heavy chain and light chain pair selected from the group consisting of SEQ ID NOS:12/14, 13/14, 15/17, and 16/17. In some embodiments, the antibody comprises six CDRs of a heavy chain and light chain pair according to SEQ ID NOS:12/14. In some embodiments, the antibody comprises six CDRs of a heavy chain and light chain pair according to SEQ ID NOS:13/14. In some embodiments, the antibody comprises six CDRs of a heavy chain and light chain pair according to SEQ ID NOS:15/17. In some embodiments, the antibody comprises six CDRs of a heavy chain and light chain pair according to SEQ ID NOS:16/17.

In some embodiments, the antibody comprises a heavy chain variable region and a light chain variable region,
wherein the heavy chain variable region comprises a CDR1 comprising the amino acid sequence of SEQ ID NO: 4; a CDR2 comprising the amino acid sequence of SEQ ID NO: 5; and a CDR3 comprises the amino acid sequence of SEQ ID NO: 6; and the light chain variable region comprising a CDR1 comprising the amino acid sequence of SEQ ID NO: 7; a CDR2 comprising the amino acid sequence of SEQ ID NO: 8; and a CDR3 comprising the amino acid sequence of SEQ ID NO: 9.

In some embodiments, the antibody comprises a heavy chain variable region and a light chain variable region,
wherein the heavy chain variable region comprises a CDR1 consisting of the amino acid sequence of SEQ ID NO: 4; a CDR2 consisting of the amino acid sequence of SEQ ID NO: 5; and a CDR3 consisting of the amino acid sequence of SEQ ID NO: 6; and the light chain variable region comprises a CDR1 consisting of the amino acid sequence of SEQ ID NO: 7; a CDR2 consisting of the amino acid sequence of SEQ ID NO: 8; and a CDR3 consisting of the amino acid sequence of SEQ ID NO: 9.

In some embodiments, the antibody comprises a heavy chain variable region of a heavy chain selected from the group consisting SEQ ID NOS:12, 13, 15, and 16, and a light chain variable region of a light chain selected from the group consisting of SEQ ID NOS:14, and 17.

In some embodiments, the antibody comprises a heavy chain variable region and a light chain variable region, wherein the heavy chain variable region comprises an amino acid sequence of SEQ ID NO: 10 and the light chain variable region comprises an amino acid sequence of SEQ ID NO: 11.

In some embodiments, the antibody comprises either one of the following heavy chain and light chain sequences:
(a) a heavy chain consisting of an amino acid sequence of SEQ ID NO: 12 and a light chain consisting of an amino acid sequence of SEQ ID NO: 14;
(b) a heavy chain consisting of an amino acid sequence of SEQ ID NO: 13 and a light chain consisting of an amino acid sequence of SEQ ID NO: 14;
(c) a heavy chain consisting of an amino acid sequence of SEQ ID NO: 15 and a light chain consisting of an amino acid sequence of SEQ ID NO: 17; or
(d) a heavy chain consisting of an amino acid sequence of SEQ ID NO: 16 and a light chain consisting of an amino acid sequence of SEQ ID NO: 17.

In some embodiments, the antibody comprises following heavy chain and light chain sequences:
(a) a heavy chain consisting of an amino acid sequence of SEQ ID NO: 12 and a light chain consisting of an amino acid sequence of SEQ ID NO: 14.

In some embodiments, the antibody comprises a heavy chain and light chain pair selected from the group consisting of SEQ ID NOS:12/14, 13/14, 15/17, and 16/17. In some embodiments, the antibody comprises a heavy chain and light chain pair according to SEQ ID NOS:12/14. In some embodiments, the antibody comprises a heavy chain and light chain pair according to SEQ ID NOS:13/14. In some embodiments, the antibody comprises a heavy chain and light chain pair according to SEQ ID NOS:15/17. In some embodiments, the antibody comprises a heavy chain and light chain pair according to SEQ ID NOS:16/17.

### 4. Non-Natural or Modified Amino Acids

When the antibody conjugate comprises a modified amino acid, the modified amino acid can be any modified amino acid deemed suitable by the practitioner. In particular embodiments, the modified amino acid comprises a reactive group useful for forming a covalent bond to a linker precursor or to a payload precursor. In certain embodiments, the modified amino acid is a non-natural amino acid. In certain embodiments, the reactive group is selected from the group consisting of amino, carboxy, acetyl, hydrazino, hydrazido, semicarbazido, sulfanyl, azido and alkynyl. Modified amino acids are also described in, for example, WO2013/185115 and WO 2015/006555, each of which is incorporated herein by reference in its entirety.

In certain embodiments, the non-natural or modified amino acid for use in the present conjugates are those described in WO 2013/185115 and WO 2015/006555, each of which is incorporated herein by reference in its entirety. In particular embodiments, the anti-TROP2 antibody or the antigen-binding fragment thereof in the antibody conjugate comprises at modified amino acid residues are at positions selected from the group consisting of heavy chain or light chain residues HC-F404, HC-K121, HC-Y180, HC-F241, HC-221, LC-T22, LC-S7, LC-N152, LC-K42, LC-E161, LC-D170, HC-S136, HC-S25, HC-A40, HC-S119, HC-S190, HC-K222, HC-R19, HC-Y52, or HC-S70, according to the Kabat or Chothia or EU numbering scheme, or a post translationally modified variant thereof.

In particular embodiments, the anti-TROP2 antibody or the antigen-binding fragment thereof in the antibody conjugate comprises at least two modified amino acid residues suitable for conjugation that are covalently linked to L^{a} and/or L^{b}. In particular embodiments, the anti-TROP2 antibody or the antigen-binding fragment thereof in the antibody conjugate comprises at least three modified amino acid residues suitable for conjugation that are covalently linked to L^{a} and/or L^{b}. In particular embodiments, the anti-TROP2 antibody or the antigen-binding fragment thereof in the antibody conjugate comprises (a) a modified amino acid residue suitable for conjugation that is covalently linked to L^{a} and (b) a modified amino acid residue suitable for conjugation that is covalently linked to L^{b} and different than the modified amino acid in (a). the antibody conjugate of the anti-TROP2 antibody or antigen-binding fragment thereof is covalently linked to L^{a} via a modified amino acid and (b) the anti-TROP2 antibody or antigen binding fragment thereof is covalently linked to L^{b} via a modified amino acid that is different than the modified amino acid in (a). In particular embodiments, the modified amino acid residue is independently selected from the group consisting of ortho-substituted tyrosine, meta substituted tyrosine, para-substituted phenylalanine, ortho-substituted phenylalanine, and meta-substituted phenylalanine. In particular embodiments, the modified amino acid residue is selected from the group consisting of p-acetyl-L-phenylalanine, O-methyl-L-tyrosine, 3-methyl-phenylalanine, O-4-allyl-L-tyrosine, 4-propyl-L-tyrosine, fluorinated phenylalanine, isopropyl-L-phenylalanine, p-azido-L-phenylalanine, p-acyl-L-phenylalanine, p-benzoyl-L-phenylalanine, p-iodo-phenylalanine, p-bromophenylalanine, p-amino-L-phenylalanine, isopropyl-L-phenylalanine, p-propargyloxy-phenylalanine, and p-azidomethyl-L-phenyl alanine. In particular embodiments, the modified amino acid residue is selected from the group consisting of p-azidomethyl-L-phenyl alanine and p-acetyl-L-phenylalanine.

In particular embodiments, provided herein are conjugates according to any of the conjugates described herein wherein Ab comprises a residue of the modified amino acid according to Formula (30), below. In particular embodiments, provided herein are conjugates according to any of the conjugates described herein wherein Ab comprises a residue of the modified amino acid according to Formula (30), below, at heavy chain position 404 according to the EU numbering system. In particular embodiments, provided herein are conjugates according to any of the conjugates described herein wherein Ab comprises a residue of the modified amino acid according to Formula (30), below, at heavy chain position 180 according to the EU numbering system. In particular embodiments, provided herein are conjugates according to any of the conjugates described herein wherein Ab comprises a residue of the modified amino acid according to Formula (30), below, at heavy chain position 241 according to the EU numbering system. In particular embodiments, provided herein are conjugates according to any of the conjugates described herein wherein Ab comprises a residue of the modified amino acid according to Formula (30), below, at heavy chain position 222 according to the EU numbering system. In particular embodiments, provided herein are conjugates according to any of the conjugates described herein wherein Ab comprises a residue of the modified amino acid according to Formula (30), below, at light chain position 7 according to the Kabat or Chothia numbering system. In particular embodiments, provided herein are conjugates according to any of the conjugates described herein wherein Ab comprises a residue of the modified amino acid according to Formula (30), below, at light chain position 42 according to the Kabat or Chothia numbering system.

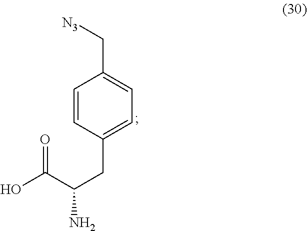 [SMILES: [N-]=[N+]=NCc1ccc(C[C@H](N)C(=O)O)cc1]

In particular embodiments, provided herein are conjugates according to any of the conjugates described herein wherein Ab comprises a residue of the modified amino acid according to Formula (65), below. In particular embodiments, provided herein are conjugates according to any of the conjugates described herein wherein Ab comprises a residue of the modified amino acid according to Formula (65), below, at heavy chain position 404 according to the EU numbering system. In particular embodiments, provided herein are conjugates according to any of the conjugates described herein wherein Ab comprises a residue of the modified amino acid according to Formula (65), below, at heavy chain position 180 according to the EU numbering system. In particular embodiments, provided herein are conjugates according to any of the conjugates described herein wherein Ab comprises a residue of the modified amino acid according to Formula (65), below, at heavy chain position 241 according to the EU numbering system. In particular embodiments, provided herein are conjugates according to any of the conjugates described herein wherein Ab comprises a residue of the modified amino acid according to Formula (65), below, at heavy chain position 222 according to the EU numbering system. In particular embodiments, provided herein are conjugates according to any of the conjugates described herein wherein Ab comprises a residue of the modified amino acid according to Formula (65), below, at light chain position 7 according to the Kabat or Chothia numbering system. In particular embodiments, provided herein are conjugates according to any of the conjugates described herein wherein Ab comprises a residue of the modified amino acid according to Formula (65), below, at light chain position 42 according to the Kabat or Chothia numbering system.

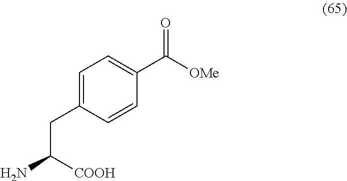 [SMILES: COC(=O)c1ccc(C[C@H](N)C(=O)O)cc1]

In particular embodiments, provided herein are conjugates according to any of the conjugates described herein wherein Ab comprises a modified amino acid residue of para-azido-L-phenylalanine. In particular embodiments, provided herein are conjugates according to any of the conjugates described herein wherein Ab comprises the modified amino acid residue para-azido-phenylalanine at heavy chain position 404 according to the EU numbering system. In particular embodiments, provided herein are conjugates according to any of the conjugates described herein wherein Ab comprises a modified amino acid residue of para-azido-L-phenylalanine at heavy chain position 180 according to the EU numbering system. In particular embodiments, provided herein are conjugates according to any of the conjugates described herein wherein Ab comprises a modified amino acid residue para-azido-L-phenylalanine at heavy chain position 241 according to the EU numbering system. In particular embodiments, provided herein are conjugates according to any of the conjugates described herein wherein Ab comprises a modified amino acid residue para-acetyl-phenylalanine at light chain position 42 according to the Kabat or Chothia numbering system. In particular embodiments, the anti-TROP2 antibody of the antibody conjugate comprises modified amino acid residues at least two positions selected from the group consisting of heavy chain position 180 according to the EU numbering system (HC-Y180), heavy chain position 241 according to the EU numbering system (HC-F241), heavy chain position 404 according to the EU numbering system (HC-F404), and light chain position 52 according to the Kabat or Chothia numbering system (LC-K52). In particular embodiments, the anti-TROP2 antibody of the antibody conjugate comprises modified amino acid residues at HC-Y180, HC-F404 and LC-K52. In particular embodiments, the anti-TROP2 antibody of the antibody conjugate comprises modified amino acid residues at HC-F241, HC-F404 and LC-K52. In one embodiment, the anti-TROP2 antibody of the antibody conjugate comprises p-acetyl-L-phenylalanine at LC-K42. In one embodiment, the anti-TROP2 antibody of the antibody conjugate comprises p-azidomethyl-L-phenyl alanine at HC-F404 and HC-F241 and p-acetyl-L-phenylalanine at LC-K42. In one embodiment, the anti-TROP2 antibody of the antibody conjugate comprises p-azidomethyl-L-phenyl alanine at HC-F404 and HC-Y180 and p-acetyl-L-phenylalanine at LC-K42. In particular embodiments, the anti-TROP2 antibody of the antibody conjugate comprises p-azidomethyl-L-phenyl alanine at HC-F404, HC-F241, and/or HC-Y180, and p-acetyl-L-phenylalanine at LC-K42. In particular embodiments, the anti-TROP2 antibody of the antibody conjugate comprises p-azidomethyl-L-phenyl alanine residues at HC-Y180 and HC-F404 and p-acetyl-L-phenylalanine residue at LC-K52. In particular embodiments, the anti-TROP2 antibody of the antibody conjugate comprises p-azidomethyl-L-phenyl alanine residues at HC-Y241 and HC-F404 and p-acetyl-L-phenylalanine residue at LC-K52. In one embodiment, the anti-TROP2 antibody of the antibody conjugate comprises p-azidomethyl-L-phenyl alanine residues at HC-Y180 and HC-F404. In particular embodiments, the anti-TROP2 antibody is post translationally modified.

In any of the foregoing embodiments, for instance wherein the antibody conjugate has a structure according to a formula described herein, for example, according to any one of Formulas 100-124 and/or Formula 201-210B, the bracketed structure can be covalently bonded to one or more non-natural or modified amino acids of the antibody, wherein the one or more non-natural or modified amino acids are located at sites independently selected from the group consisting of: HC-F241, HC-F404, HC-Y180, and LC-K42, and combinations thereof, according to the Kabat or EU numbering scheme of Kabat. In some embodiments, the bracketed structure is covalently bonded to one or more non-natural or modified amino acids at site HC-F404 of the antibody. In some embodiments, the bracketed structure is covalently bonded to one or more non-natural or modified amino acids at site HC-Y180 of the antibody. In some embodiments, the bracketed structure is covalently bonded to one or more non-natural or modified amino acids at site HC-F241 of the antibody. In some embodiments, the bracketed structure is covalently bonded to one or more non-natural or modified amino acids at site LC-K42 of the antibody. In some embodiments, the bracketed structure is covalently bonded to one or more non-natural or modified amino acids at sites HC-F404 and HC-Y180 of the antibody. In some embodiments, the bracketed structure is covalently bonded to one or more non-natural or modified amino acids at sites HC-F404 and HC-Y180 of the antibody, and the other bracketed structure is bonded to a non-natural or modified amino acids at site LC-K42 of the antibody. In some embodiments, the bracketed structure is covalently bonded to one or more non-natural or modified amino acids at sites HC-F404 and HC-F241 of the antibody. In some embodiments, the bracketed structure is covalently bonded to one or more non-natural or modified amino acids at sites HC-F404 and HC-F241 of the antibody, and the other bracketed structure is bonded to a non-natural or modified amino acids at site LC-K42 of the antibody. In some embodiments, the bracketed structure is covalently bonded to one or more non-natural or modified amino acids at sites HC-F241, HC-F404, and HC-Y180 of the antibody.

In some embodiments, the bracketed structure is covalently bonded to one or more non-natural or modified amino acids at sites HC-Y180 and LC-K42 of the antibody. In some embodiments, the bracketed structure is covalently bonded to one or more non-natural or modified amino acids at sites HC-F404 and LC-K42 of the antibody. In some embodiments, the bracketed structure is covalently bonded to one or more non-natural or modified amino acids at sites HC-F404, HC-Y180, and LC-K42 of the antibody. In some embodiments, one bracketed structure binds to L-para-acetyl-phenylalanine (pAcF) at one or more sites and the other bracketed structure binds to a residue according to L-para-azido-phenylalanine (pAMF) at one or more sites. In one embodiment of Formula (I), Ab is the anti-TROP2 antibody of the antibody conjugate and Ab comprises p-azidomethyl-L-phenyl alanine residues at HC-Y180 and HC-F404 and [CY-L^{a}-]ₘ- is bonded to the p-azidomethyl-L-phenyl alanine residues at HC-Y180 and HC-F404. In one embodiment of Formula (I), including any of the foregoing, Ab is the anti-TROP2 antibody of the antibody conjugate and Ab comprises p-acetyl-L-phenylalanine at LC-K42 and -[L_{b}-STING]₁ is bonded to the p-acetyl-L-phenylalanine at LC-K42.

In particular embodiments of Formula (I), Ab is the anti-TROP2 antibody of the antibody conjugate and Ab comprises p-azidomethyl-L-phenyl alanine residues at HC-Y180 and HC-F404 and p-acetyl-L-phenylalanine residue at LC-K42, [CY-L^{a}-]ₘ- is bonded to the p-azidomethyl-L-phenyl alanine residues at HC-Y180 and HC-F404, and -[L_{b}-STING]ₙ is bonded to the p-acetyl-L-phenylalanine at LC-K42.

In some embodiments, the bracketed structure is covalently bonded to one or more non-natural or modified amino acids at sites HC-F241 and LC-K42 of the antibody. In some embodiments, the bracketed structure is covalently bonded to one or more non-natural or modified amino acids at sites HC-F404 and LC-K42 of the antibody. In some embodiments, the bracketed structure is covalently bonded to one or more non-natural or modified amino acids at sites HC-F404, HC-F241, and LC-K42 of the antibody. In some embodiments, one bracketed structure binds to L-para-acetyl-phenylalanine (pAcF) at one or more sites and the other bracketed structure binds to a residue according to L-para-azido-phenylalanine (pAMF) at one or more sites. In particular embodiments of Formula (I), Ab is the anti-TROP2 antibody of the antibody conjugate and Ab comprises p-azidomethyl-L-phenyl alanine residues at HC-F241 and HC-F404 and p-acetyl-L-phenylalanine residue at LC-K42, [CY-L^{a}-]ₘ- is bonded to the p-azidomethyl-L-phenyl alanine residues at HC-F241 and HC-F404, and -[L^{b}-STING]ₙ is bonded to the p-acetyl-L-phenylalanine at LC-K42.

In some embodiments, the antibody conjugate binding to the anti-TROP2 antibody is a conjugate wherein

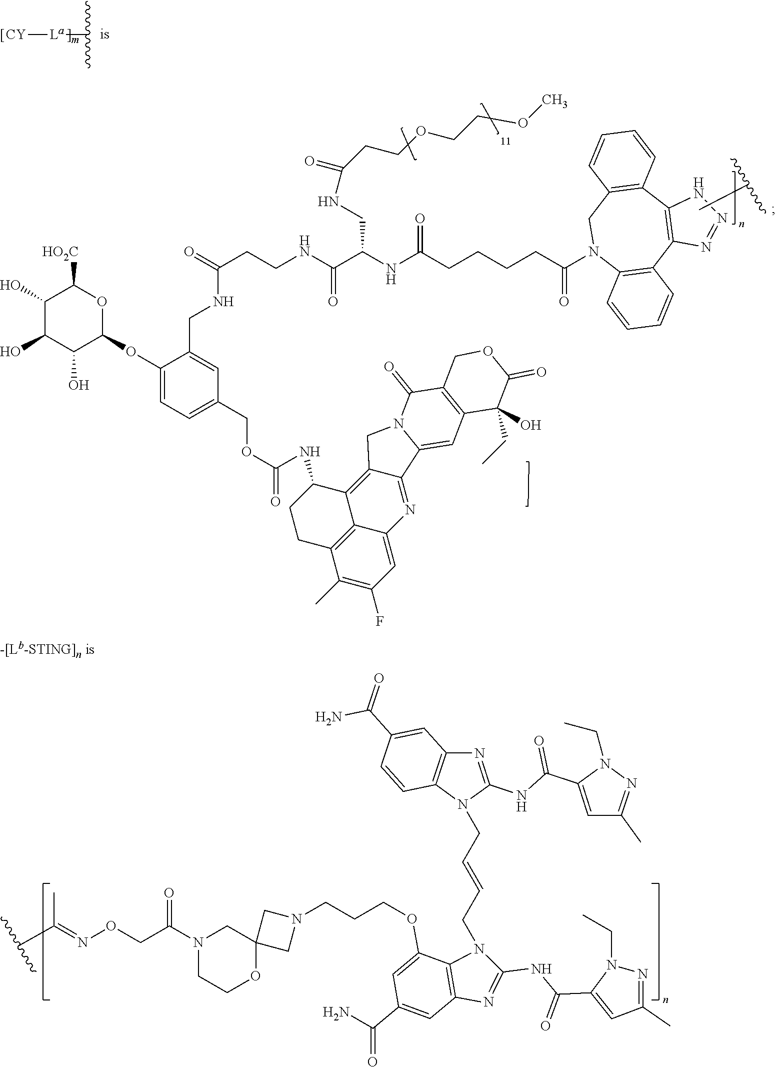

wherein the anti-TROP2 antibody comprises a heavy chain consisting of an amino acid sequence of SEQ ID NO: 12 and a light chain consisting of an amino acid sequence of SEQ ID NO: 14 wherein the antibody comprises p-azidomethyl-L-phenyl alanine residues at HC-Y180 and HC-F404 and p-acetyl-L-phenylalanine residue at LC-K42, [CY-L^{a}-]ₘ- is bonded to the p-azidomethyl-L-phenyl alanine residues at HC-Y180 and HC-F404, and -[L_{b}-STING]ₙ is bonded to the p-acetyl-L-phenylalanine at LC-K42.

In some embodiments, the antibody conjugate binding to the anti-TROP2 antibody is a conjugate wherein

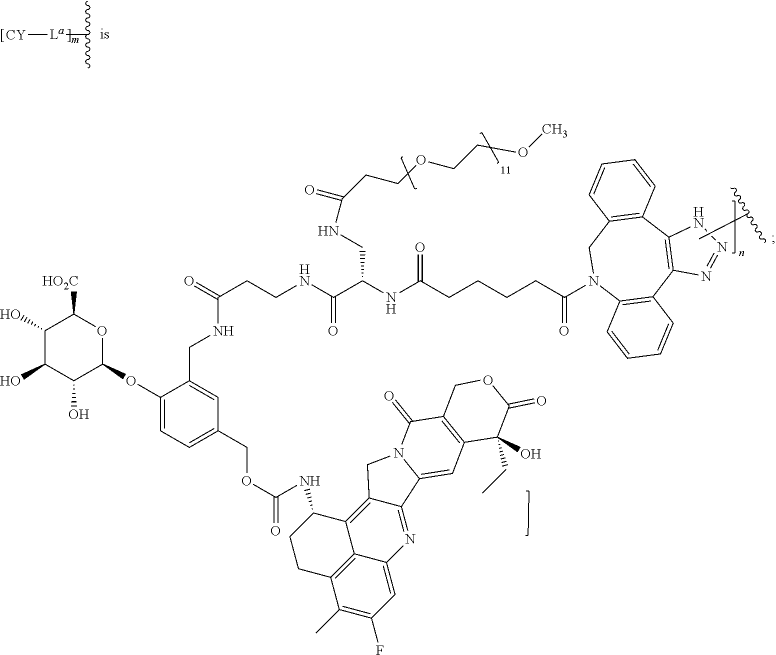

and
-[L^{b}-STING]ₙ is a structure selected from

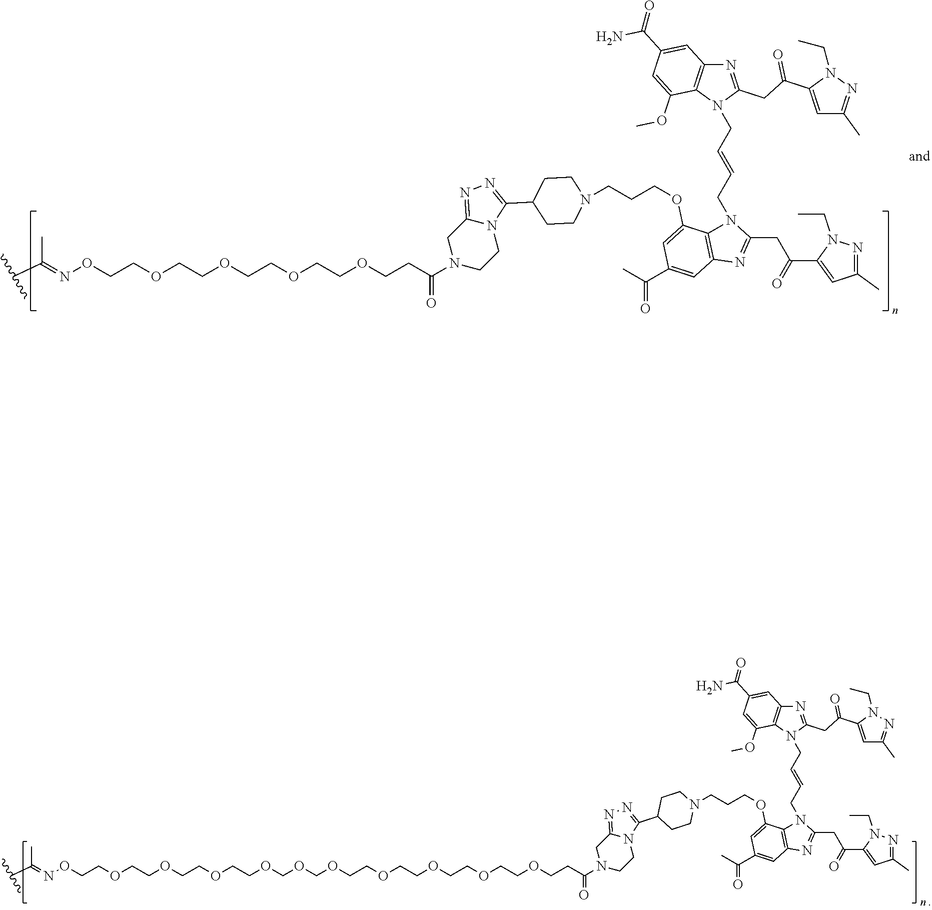

### Optically Active Compounds

In certain embodiments, compounds, linker-payloads, and conjugates provided herein may have several chiral centers and may exist in and be isolated in optically active and racemic forms. In certain embodiments, some compounds, linker-payloads, or conjugates may exhibit polymorphism. A person of skill in the art will appreciate that compounds, linker-payloads, and conjugates provided herein can exist in any racemic, optically-active, diastereomeric, polymorphic, regioisomeric and/or stereoisomeric form, and/or mixtures thereof.

A person of skill in the art will also appreciate that such compounds, linker-payloads, and conjugates described herein that possess the useful properties also described herein are within the scope of this disclosure. A person of skill in the art will further appreciate how to prepare optically active forms of the compounds, linker-payloads, and conjugates described herein, for example, by resolution of racemic forms via recrystallization techniques, by synthesis from optically-active starting materials, by chiral synthesis, or by chromatographic separation using a chiral stationary phase. In addition, most amino acids are chiral (i.e., designated as L- or D-, wherein the L-enantiomer is the naturally occurring configuration) and can exist as separate enantiomers.

Examples of methods to obtain optically active materials are known in the art, and include at least the following:
physical separation of crystals—a technique whereby macroscopic crystals of the individual enantiomers are manually separated. This technique can be used if crystals of the separate enantiomers exist (i.e., the material is a conglomerate, and the crystals are visually distinct);
ii) simultaneous crystallization—a technique whereby the individual enantiomers are separately crystallized from a solution of the racemate, only if the latter is a conglomerate in the solid state;
iii) enzymatic resolutions—a technique wherein partial or complete separation of a racemate is accomplished by virtue of different rates of reaction of the enantiomers in the presence of an enzyme;
iv) enzymatic asymmetric synthesis—a synthetic technique wherein at least one step of the synthesis uses an enzymatic reaction to obtain an enantiomerically pure or enriched synthetic precursor of the desired enantiomer;
v) chemical asymmetric synthesis—a synthetic technique wherein the desired enantiomer is synthesized from an achiral precursor using chiral catalysts or chiral auxiliaries to produce asymmetry (i.e., chirality) in the product;
vi) diastereomer separations—a technique wherein a racemic compound is treated with an enantiomerically pure reagent (a chiral auxiliary) that converts the individual enantiomers to diastereomers. The resulting diastereomers are then separated by chromatography or crystallization by virtue of their now more distinct diastereomeric differences, and then the chiral auxiliary is removed to obtain each enantiomer;
vii) first- and second-order asymmetric transformations—a technique wherein diastereomers of the racemate equilibrate in solution to yield a preponderance of a diastereomer of the desired enantiomer, or where kinetic or thermodynamic crystallization of the diastereomer of the desired enantiomer perturbs the equilibrium such that eventually in principle all the material is converted to the crystalline diastereomer of the desired enantiomer. The desired enantiomer is then derived from the diastereomer;
viii) kinetic resolutions—this technique refers to the achievement of partial or complete resolution of a racemate (or of a further resolution of a partially resolved compound) by virtue of unequal reaction rates of the enantiomers with a chiral or non-racemic reagent or catalyst under kinetic conditions;
ix) enantiospecific synthesis from non-racemic precursors—a synthetic technique wherein the desired enantiomer is obtained from chiral starting materials and where the stereochemical integrity is not or is only minimally compromised over the course of the synthesis;
x) chiral liquid chromatography—a technique wherein the enantiomers of a racemate are separated in a liquid mobile phase by virtue of their different interactions with a stationary phase. The stationary phase can be made of chiral material or the mobile phase can contain an additional chiral material to provoke the different interactions;
xi) chiral gas chromatography—a technique wherein the racemate is volatilized and enantiomers are separated by virtue of their different interactions in the gaseous mobile phase with a column containing a fixed non-racemic adsorbent phase;
xii) extraction with chiral solvents—a technique wherein the enantiomers are separated by virtue of kinetic or thermodynamic dissolution of one enantiomer into a particular chiral solvent;
a) transport across chiral membranes—a technique wherein a racemate is placed in contact with a thin membrane barrier. The barrier typically separates two miscible fluids, one containing the racemate, and a driving force such as a concentration or pressure differential causes preferential transport across the membrane barrier. Separation occurs as a result of the non-racemic nature of the membrane which allows only one enantiomer of the racemate to pass through.

In some embodiments, provided herein are compositions of the compounds, linker-payloads, or conjugates of the present disclosure that are substantially free of a designated stereoisomer of that compound, linker-payload, or conjugate, respectively. In certain embodiments, in the methods, compounds, linker-payloads, and conjugates of this disclosure, the compounds, linker-payloads, or conjugates are substantially free of other stereoisomers. In some embodiments, the composition includes a compound, linker-payload, or conjugate that is at least 85%, 90%, 95%, 98%, or 99% to 100% by weight of the compound, linker-payload or conjugate, respectively, the remainder comprising other chemical species or enantiomers. In some embodiments, provided herein are compositions of conjugates, linker-payloads, or compounds described herein that are substantially free of a designated enantiomer of that conjugate, linker-payload, or compound, respectively. In certain embodiments, in the methods, compounds, linker-payloads, and conjugates of this disclosure, the compounds, linker-payloads, or conjugates are substantially free of other enantiomers. In some embodiments, the composition includes a compound, linker-payload, or conjugate that is at least 85%, 90%, 95%, 98%, or 99% to 100% by weight of the compound, linker-payload, or conjugate, respectively, the remainder comprising other chemical species or enantiomers.

### Isotopically Enriched Compounds

Also provided herein are isotopically enriched compounds, linker-payloads, and conjugates of the compounds, linker-payloads, and conjugates described herein.

Isotopic enrichment (for example, deuteration) of pharmaceuticals to improve pharmacokinetics (“PK”), pharmacodynamics (“PD”), and/or toxicity profiles, has been previously demonstrated within some classes of drugs. See, for example, Lijinsky et al., Food Cosmet. Toxicol., 20: 393 (1982); Lijinsky et al., J. Nat. Cancer Inst., 69: 1127 (1982); Mangold et al., Mutation Res. 308: 33 (1994); Gordon et al., Drug Metab. Dispos., 15: 589 (1987); Zello et al., Metabolism, 43: 487 (1994); Gately et al., J. Nucl. Med., 27: 388 (1986); Wade D, Chem. Biol. Interact. 117: 191 (1999).

Isotopic enrichment of a drug can be used, for example, to (1) reduce or eliminate unwanted metabolites; (2) increase the half-life of the parent drug; (3) decrease the number of doses needed to achieve a desired effect; (4) decrease the amount of a dose necessary to achieve a desired effect; (5) increase the formation of active metabolites, if any are formed; and/or (6) decrease the production of deleterious metabolites in specific tissues. Isotopic enrichment of a drug can also be used to create a more effective and/or safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a (heavier) isotope for that reactive hydrogen will cause a decrease in the reaction rate. The Deuterium Kinetic Isotope Effect (“DKIE”) is the most common form of KIE. (See, e.g., Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen and the C—D bond is broken. The DKIE can range from about one (no isotope effect) to very large numbers, such as 50 or more, meaning that the reaction can be fifty, or more, times slower when deuterium has been substituted for hydrogen.

Substitution of tritium (“T”) for hydrogen results in yet a stronger bond than deuterium and gives numerically larger isotope effects. Similarly, substitution of isotopes for other elements including, but not limited to, ¹³C or ¹⁴C for carbon; ³³S, ³⁴S, or ³⁶S for sulfur; ¹⁵N for nitrogen; and ¹⁷O or ¹⁸O for oxygen may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C═C) pi-bond. The resultant metabolites may be stable or unstable under physiological conditions, and can have substantially different PK/PD, and acute and long-term toxicity profiles relative to the parent compounds. For many drugs, such oxidations are rapid. Therefore, these drugs often require the administration of multiple or high daily doses.

Therefore, isotopic enrichment at certain positions of a compound provided herein will produce a detectable KIE that will affect the pharmacologic, PK, PD, and/or toxicological profiles of a compound provided herein in comparison with a similar compound having a natural isotopic composition.

### Preparation of Antibodies and Antibody Conjugates

### Antigen Preparation

The protein to be used for isolation of the antibodies may be intact antigen or a fragment of antigen. The intact protein, or fragment of the antigen, may be in the form of an isolated protein or protein expressed by a cell. Other forms of antigens useful for generating antibodies will be apparent to those skilled in the art.

### Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

### Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### Conjugation

The antibody conjugates can be prepared by standard techniques. In certain embodiments, an antibody is contacted with a payload precursor under conditions suitable for forming a bond from the antibody to the payload to form an antibody-payload conjugate. In certain embodiments, an antibody is contacted with a linker precursor under conditions suitable for forming a bond from the antibody to the linker. The resulting antibody-linker is contacted with a payload precursor under conditions suitable for forming a bond from the antibody-linker to the payload to form an antibody-linker-payload conjugate. In certain embodiments, a payload precursor is contacted with a linker precursor under conditions suitable for forming a bond from the payload to the linker. The resulting payload-linker is contacted with an antibody under conditions suitable for forming a bond from the payload-linker to the antibody to form an antibody-linker-payload conjugate. Suitable linkers for preparing the antibody conjugates are disclosed herein, and exemplary conditions for conjugation are described in the Examples below.

In some embodiments, the conjugate is prepared by contacting an antibody disclosed herein with 1) a linker precursor having a structure of any of the following STING linker payloads:

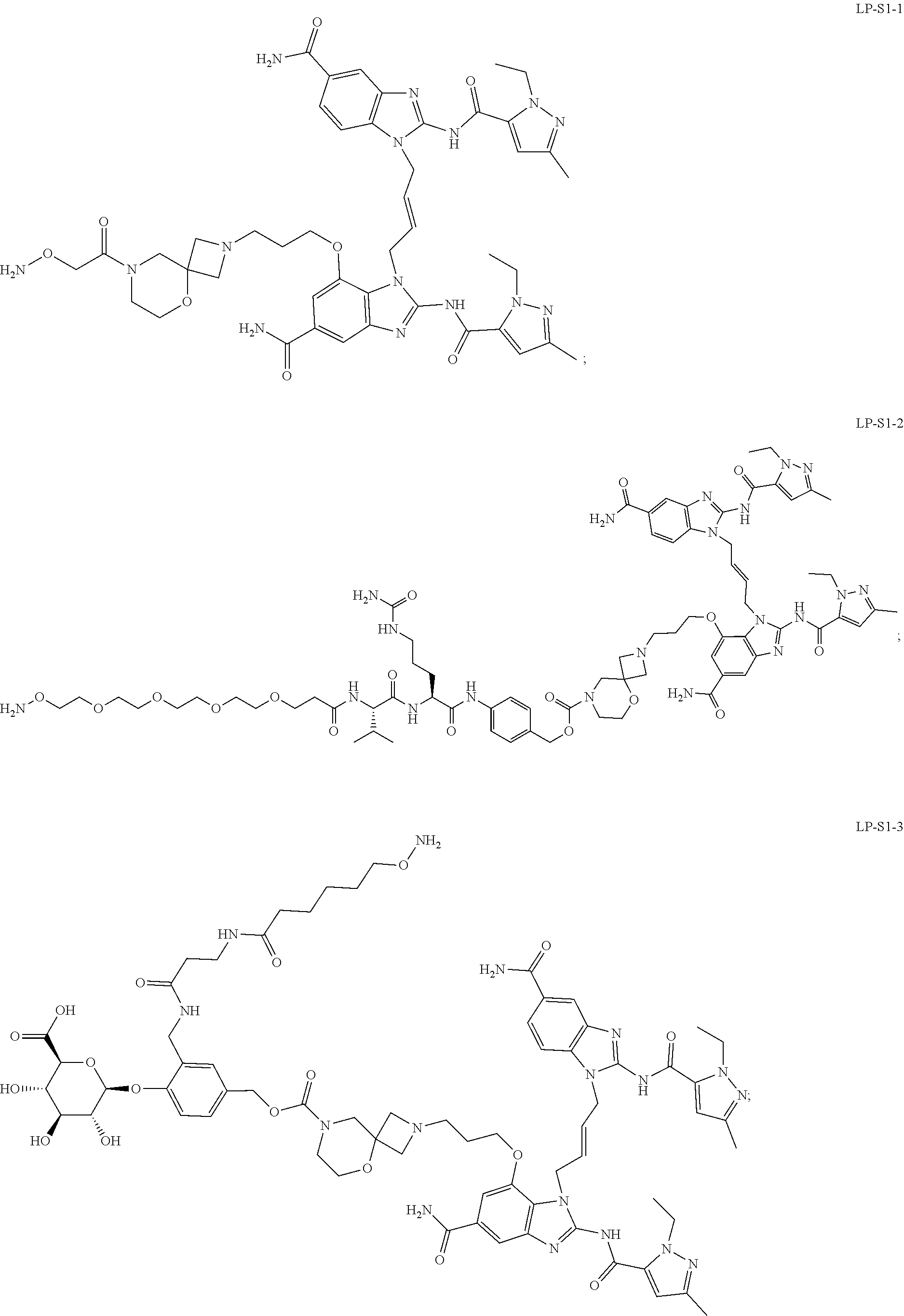

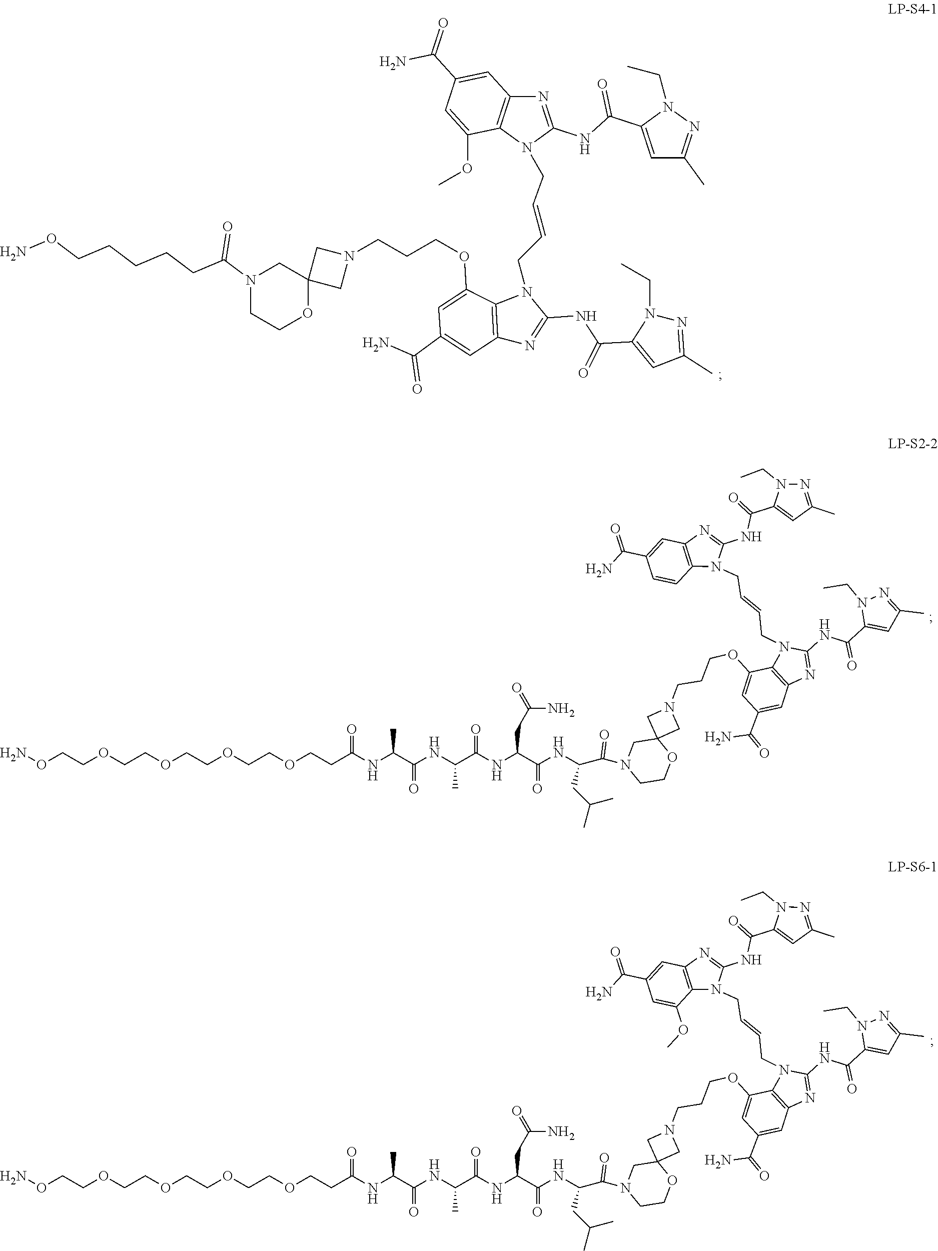

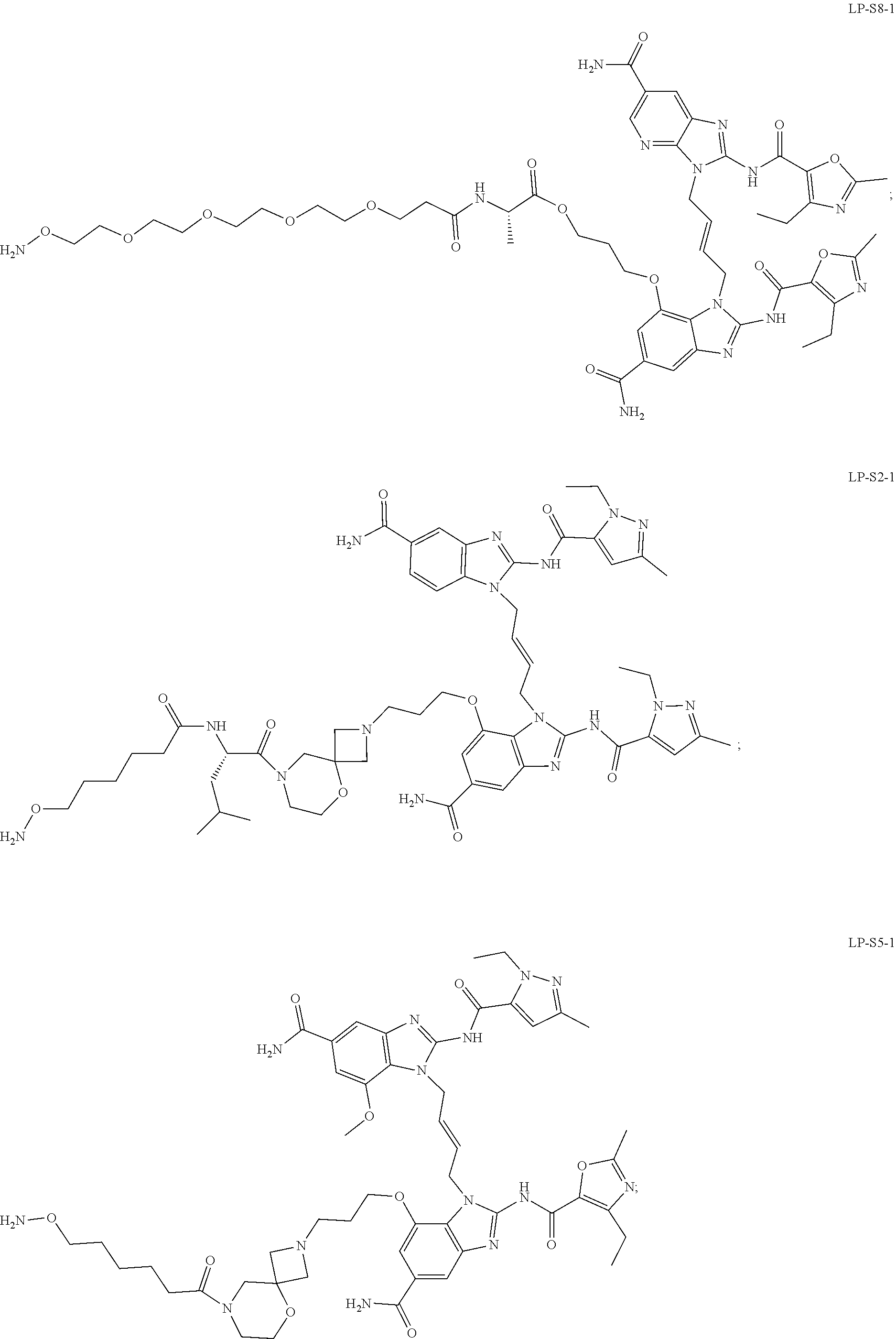 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCOC(=O)[C@H](C)NC(=O)CCOCCOCCOCCOCCON)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)CCCCCON)CCO4)c21 ; CCc1nc(C)oc1C(=O)Nc1nc2cc(C(N)=O)cc(OCCCN3CC4(C3)CN(C(=O)CCCCCON)CCO4)c2n1C/C=C/Cn1c(NC(=O)c2cc(C)nn2CC)nc2cc(C(N)=O)cc(OC)c21]

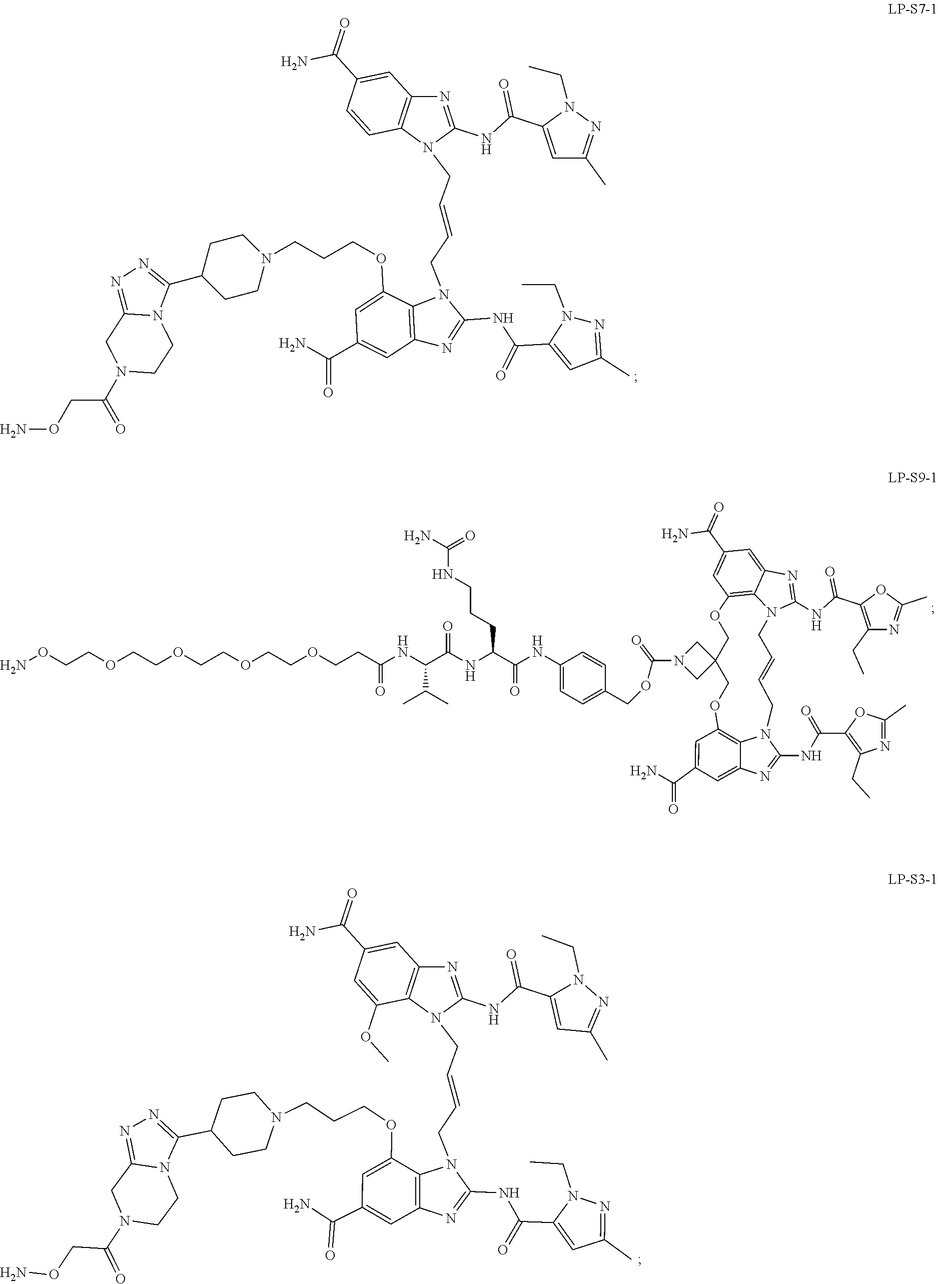 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCC(c4nnc5n4CCN(C(=O)CON)C5)CC3)c21 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)[C@H](CCCNC(N)=O)NC(=O)[C@@H](NC(=O)CCOCCOCCOCCOCCON)C(C)C)cc2)C1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

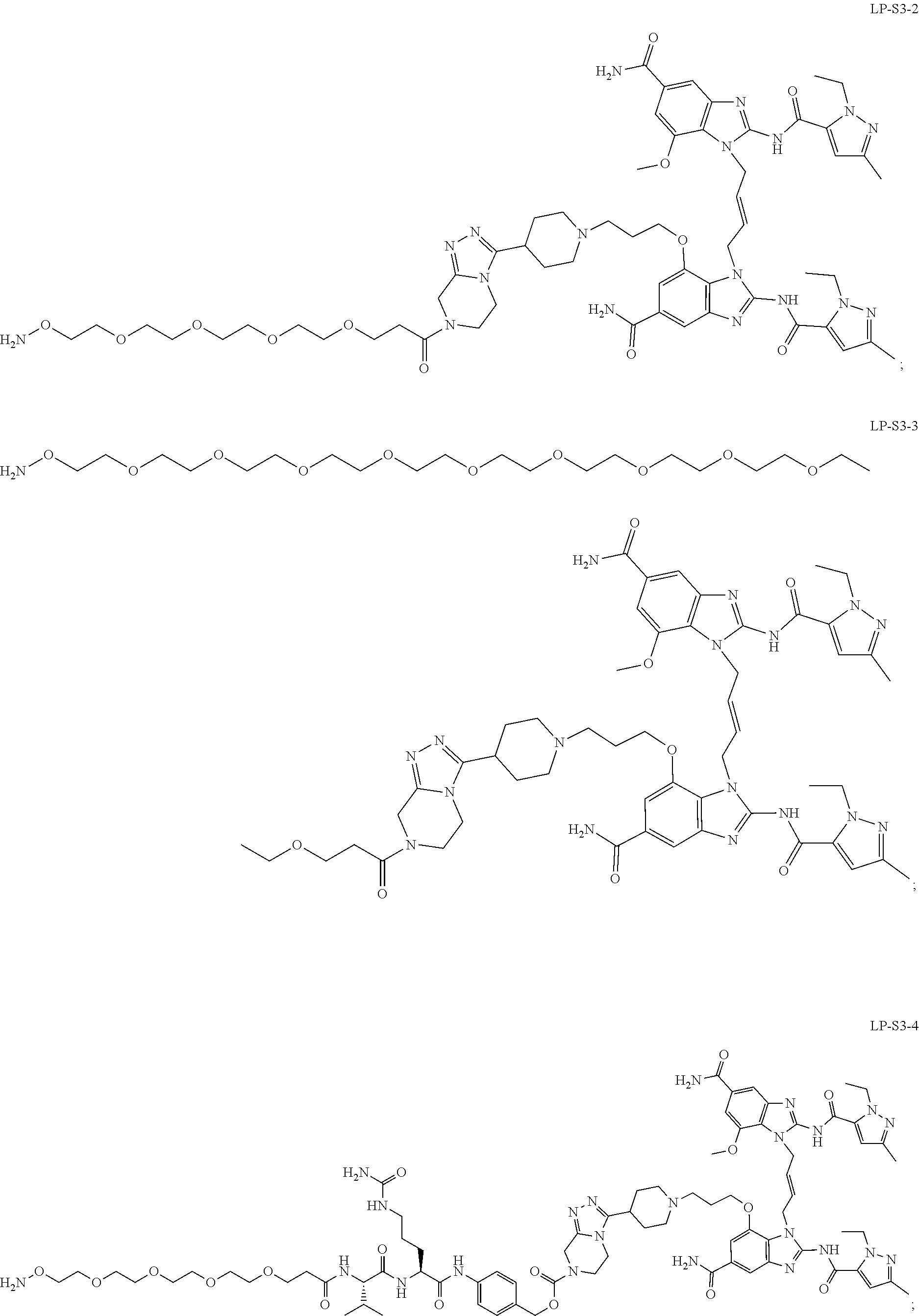

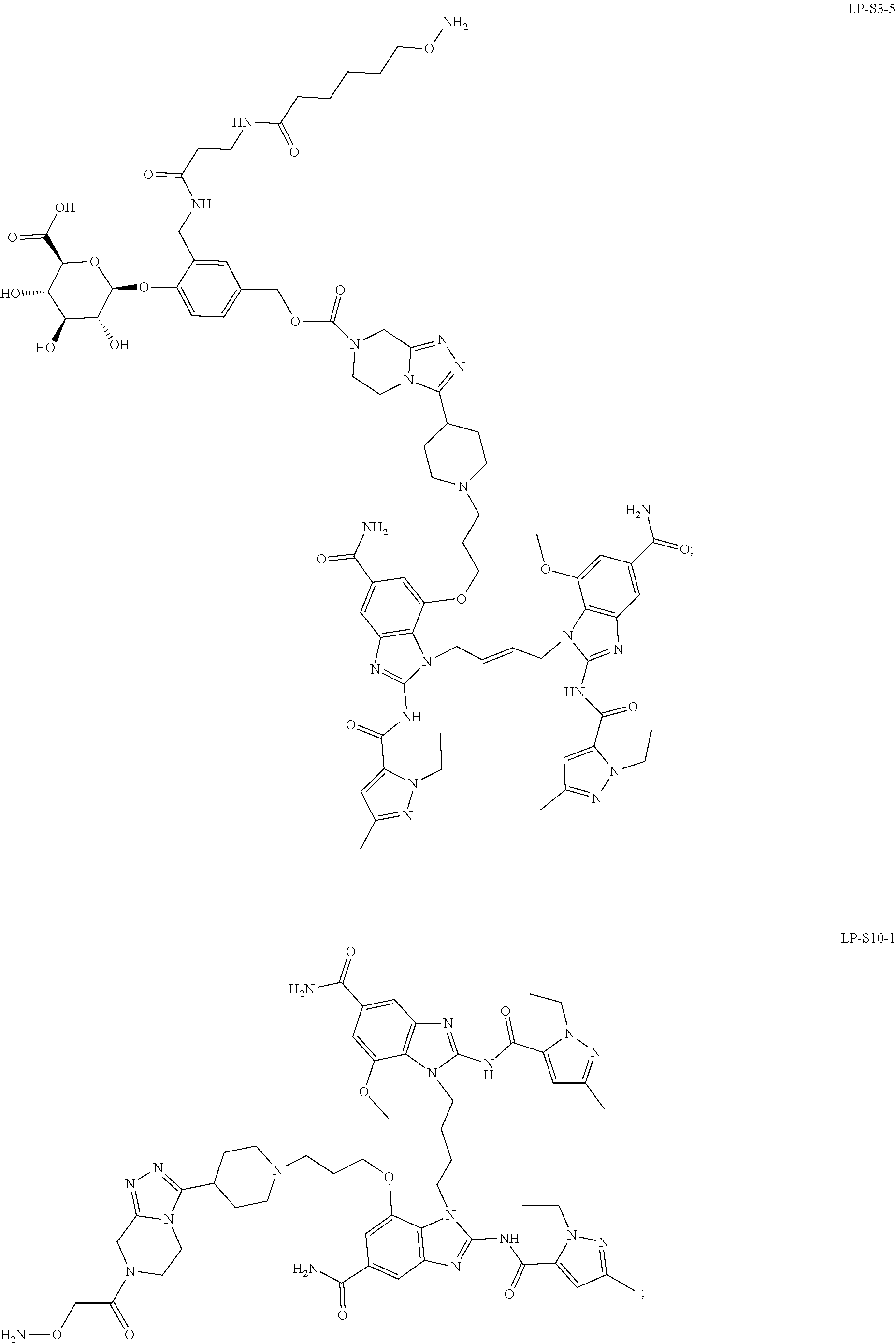 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](O)[C@H](O)[C@H]6O)c(CNC(=O)CCNC(=O)CCCCCON)c4)C5)CC3)c21 ; CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)CON)C5)CC3)c21]

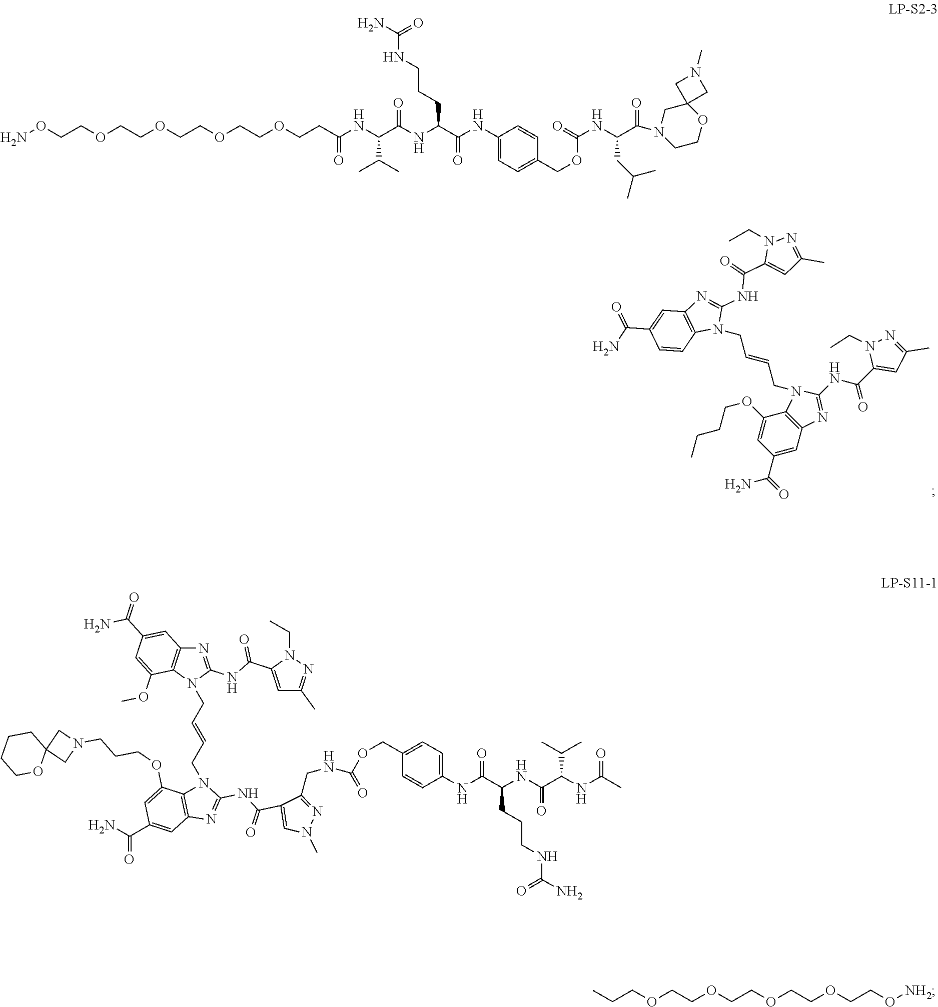

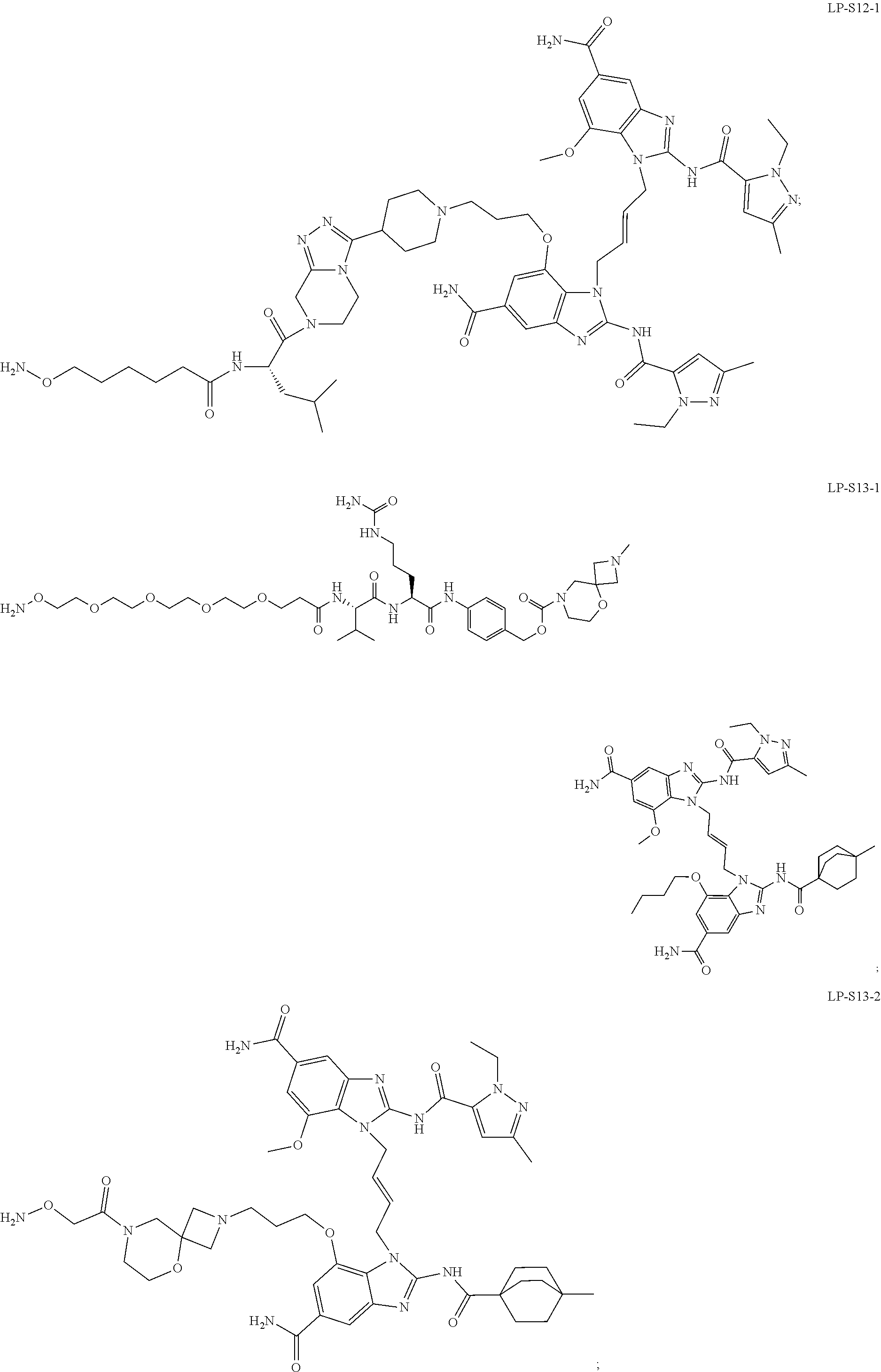 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCON)C5)CC3)c21 ; CC(C)[C@H](NC(=O)CCOCCOCCOCCOCCON)C(=O)N[C@@H](CCCNC(N)=O)C(=O)Nc1ccc(COC(=O)N2CCOC3(CN(C)C3)C2)cc1 ; CCCCOc1cc(C(N)=O)cc2nc(NC(=O)C34CCC(C)(CC3)CC4)n(C/C=C/Cn3c(NC(=O)c4cc(C)nn4CC)nc4cc(C(N)=O)cc(OC)c43)c12 ; CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)CON)CCO4)c21]

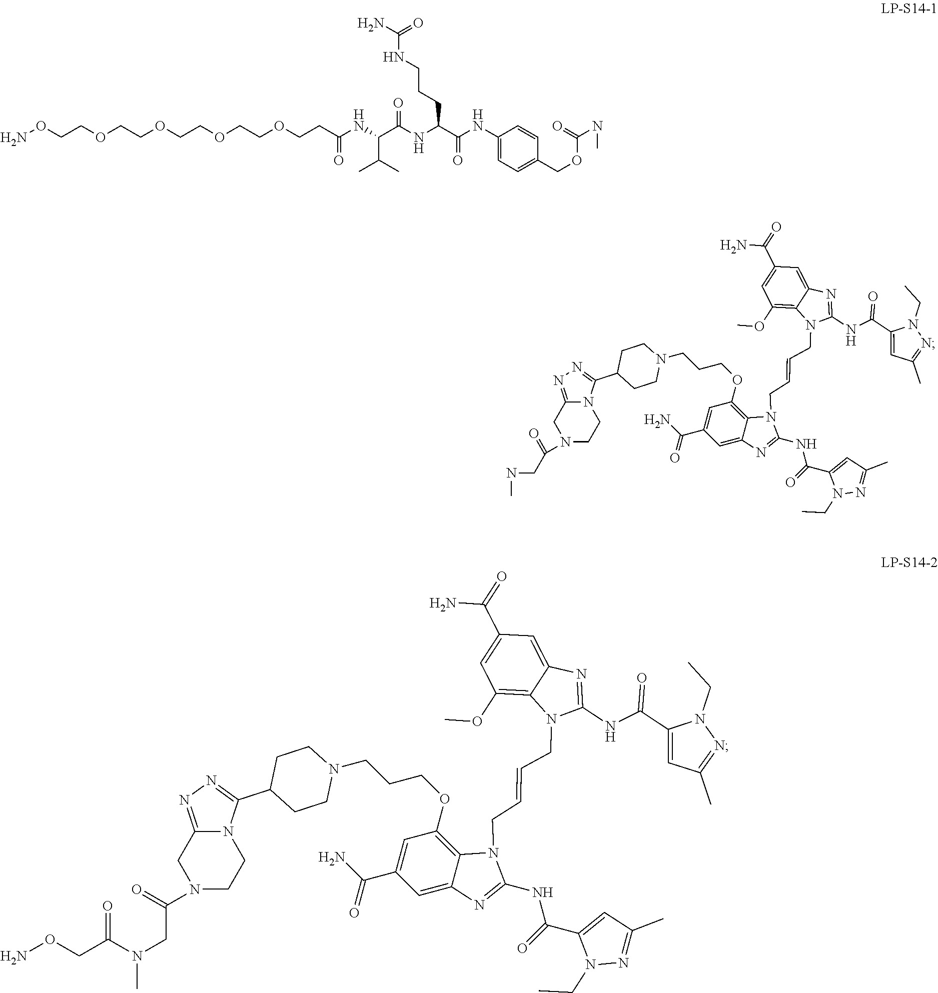 [SMILES: CNC(=O)OCc1ccc(NC(=O)[C@H](CCCNC(N)=O)NC(=O)[C@@H](NC(=O)CCOCCOCCOCCOCCON)C(C)C)cc1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CNC)C5)CC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)CON)C5)CC3)c21]

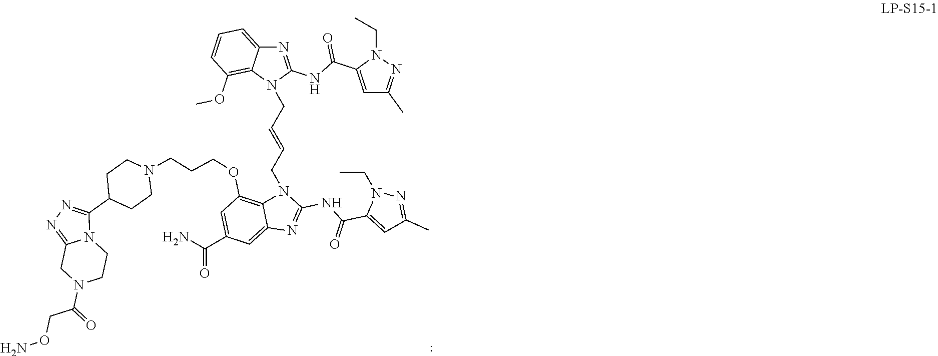 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

or a pharmaceutically acceptable salt, solvate, stereoisomer, regioisomer, or mixture of regioisomers thereof; and 2) a linker precursor having a structure of any of the following cytotoxin linker payloads:

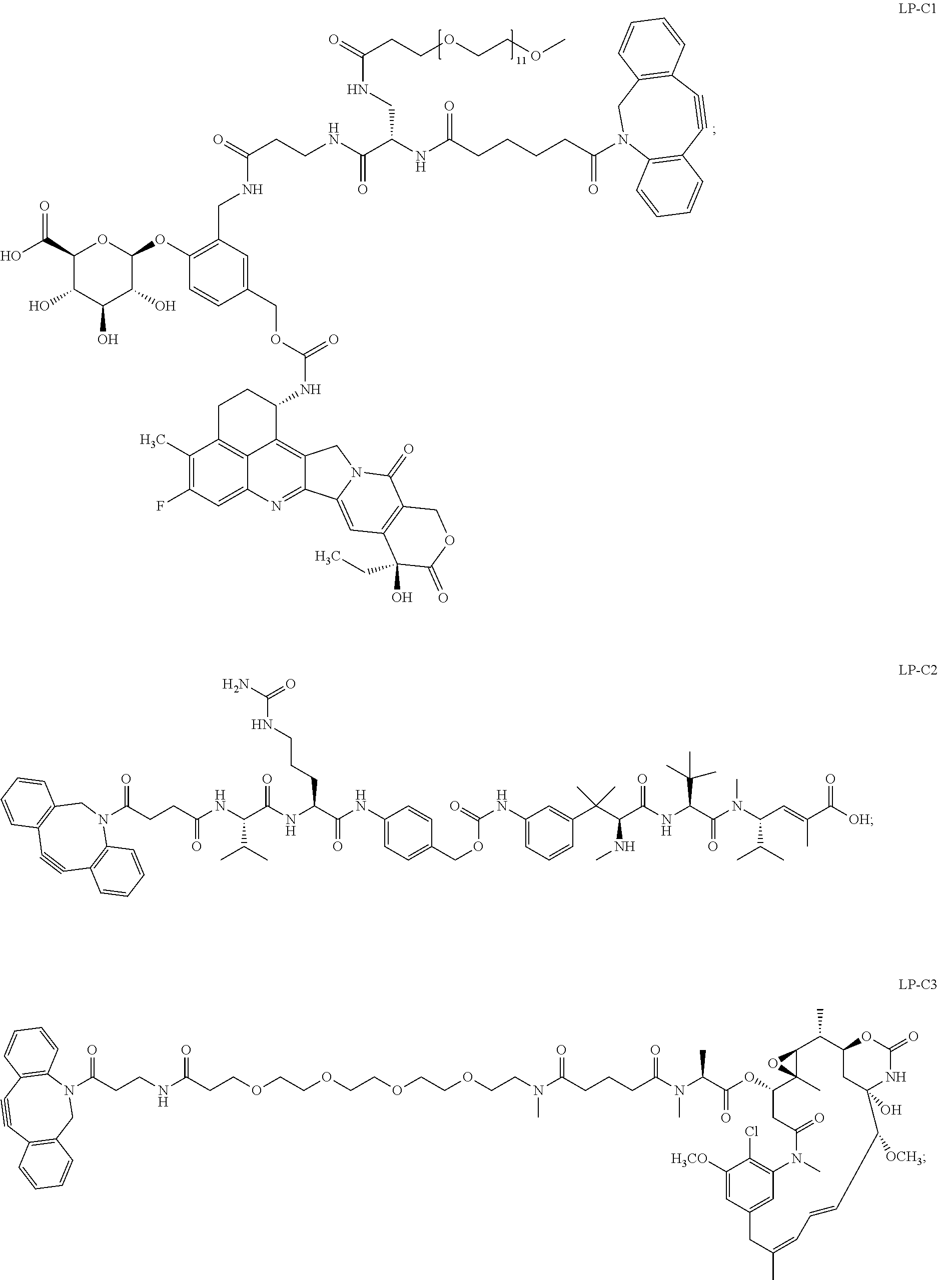

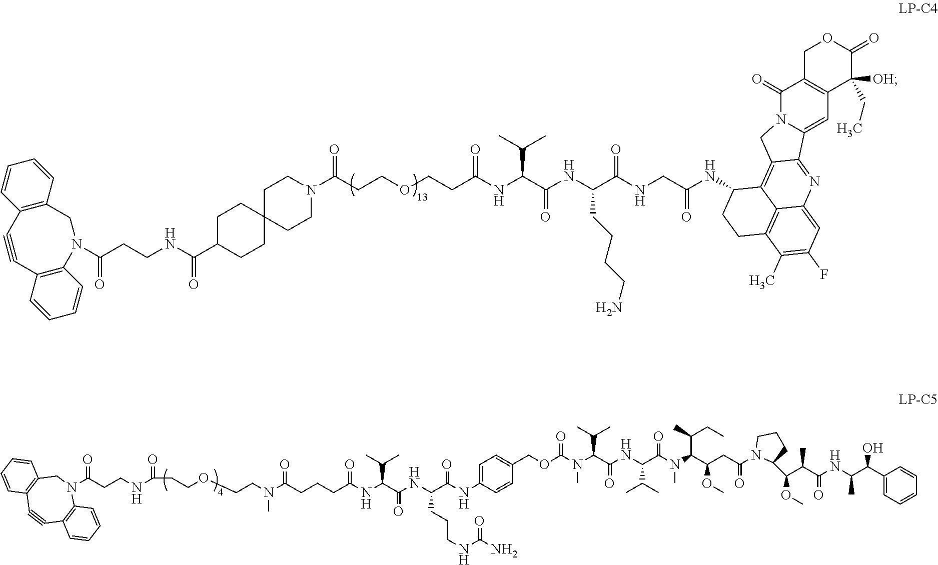

or a pharmaceutically acceptable salt, solvate, stereoisomer, regioisomer, or mixture of regioisomers thereof.

### Reactive Groups

Reactive groups facilitate conjugation of the compounds of linker-payloads as described herein to the antibody or antigen binding fragment described herein to form a conjugate of Formula (I), (Ia), (Ib), (Ic), (Ib-1), (Ib-2), (Ic-1), or (Ic-2) as described herein. Reactive groups can react via any suitable reaction mechanism known to those of skill in the art. In certain embodiments, a reactive group reacts through a [3+2]alkyne-azide cycloaddition reaction, inverse-electron demand Diels-Alder ligation reaction, thiol-electrophile reaction, or carbonyl-oxyamine reaction, as described in detail herein. In certain embodiments, the reactive group comprises an alkyne, strained alkyne, tetrazine, thiol, para-acetyl-phenylalanine residue, oxyamine, maleimide, or azide. In certain embodiments, the reactive group is

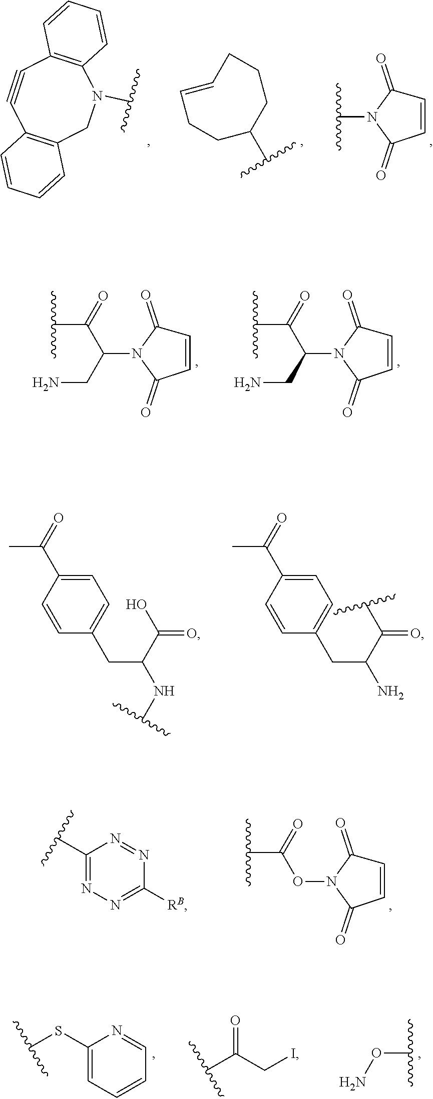

—N₃, or —SH; wherein R^{B} is lower alkyl. In certain embodiments, R^{B} is methyl, ethyl, or propyl. In some embodiments, R^{B} is methyl. In some embodiments, R^{T} is ethyl. In some embodiments, R^{B} is propyl. Additional reactive groups are described in, for example, U.S. Patent Application Publication No. 2014/0356385, U.S. Patent Application Publication No. 2013/0189287, U.S. Patent Application Publication No. 2013/0251783, U.S. Pat. Nos. 8,703,936, 9,145,361, 9,222,940, and 8,431,558.

In certain embodiments, the reactive group is

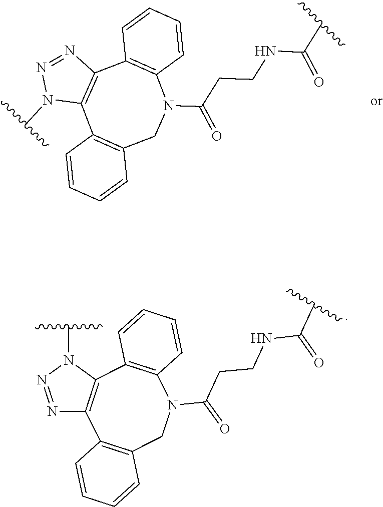 [SMILES: *C(=O)NCCC(=O)N1Cc2ccccc2-c2c(nnn2*)-c2ccccc21 ; *C(=O)NCCC(=O)N1Cc2ccccc2-c2nnn(*)c2-c2ccccc21]

In certain embodiments, the reactive group is

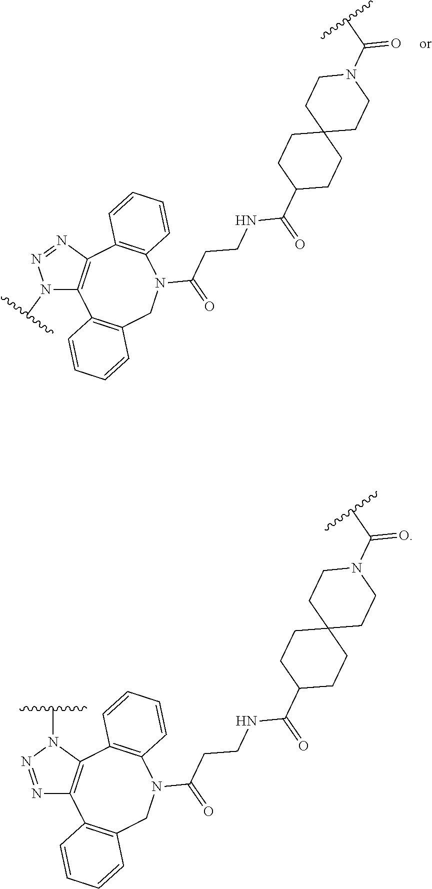

After conjugation, a divalent residue of the reactive group (referred to as RL herein) is formed and is bonded to antibody or antigen binding fragment thereof. The structure of the divalent residue is determined by the type of conjugation reaction employed to form the conjugate.

### [3+2] Alkyne-Azide Cycloaddition Reaction

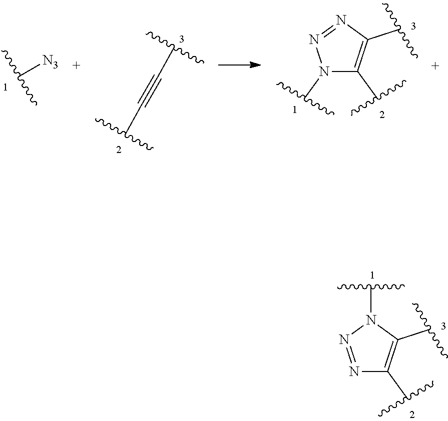

Advantageously, the compounds described herein comprising a conjugating alkyne group or an azide group facilitate selective and efficient reactions with a second compound comprising a complementary azide group or alkyne group. It is believed the azide and alkyne groups react in a 1,3-dipolar cycloaddition reaction to form a 1,2,3-triazolylene moiety which links the compounds described herein comprising an alkyne group or an azide group to the second compound. This reaction between an azide and alkyne to form a triazole is generally known to those in the art as a Huisgen cycloaddition reaction or a [3+2]alkyne-azide cycloaddition reaction.

The unique reactivity of azide and alkyne functional groups makes them useful for the selective modification of polypeptides and other biological molecules. Organic azides, particularly aliphatic azides, and alkynes are generally stable toward common reactive chemical conditions. In particular, both the azide and the alkyne functional groups are inert toward the side chains of the twenty common amino acids found in naturally-occurring polypeptides. It is believed that, when brought into close proximity, the “spring-loaded” nature of the azide and alkyne groups is revealed and azide and alkyne groups react selectively and efficiently via a [3+2] alkyne-azide cycloaddition reaction to generate the corresponding triazole. See, e.g., Chin J., et al., Science 301:964-7 (2003); Wang, Q., et al., J Am. Chem. Soc. 125, 3192-3193 (2003); Chin, J. W., et al., J. Am. Chem. Soc. 124:9026-9027 (2002).

Because the [3+2] alkyne-azide cycloaddition reaction involves a selective cycloaddition reaction [see, e.g., Padwa, A., in COMPREHENSIVE ORGANIC SYNTHESIS, Vol. 4, (ed. Trost, B. M., 1991), pp. 1069-1109; Huisgen, R. in 1,3-DIPOLAR CYCLOADDITION CHEMISTRY, (ed. Padwa, A., 1984), pp. 1-176] rather than a nucleophilic substitution, the incorporation of non-natural or modified amino acids bearing azide and alkyne-containing side chains permits the resultant polypeptides to be modified selectively at the position of the non-natural or modified amino acid. Cycloaddition reactions involving azide or alkyne-containing compounds can be carried out at room temperature under aqueous conditions by the addition of Cu(II) (including, but not limited to, catalytic amounts of CuSO₄) in the presence of a reducing agent for reducing Cu(II) to Cu(I), in situ, in catalytic amounts. See, e.g., Wang, Q., et al., J. Am. Chem. Soc. 125, 3192-3193 (2003); Tornoe, C. W., et al., J. Org. Chem. 67:3057-3064 (2002); Rostovtsev, et al., Angew. Chem. Int. Ed. 41:2596-2599 (2002). Exemplary reducing agents include, but are not limited to, ascorbate, metallic copper, quinine, hydroquinone, vitamin K, glutathione, cysteine, Fe²⁺, Co²⁺, and an applied electric potential.

In certain embodiments when a conjugate is formed through a [3+2]alkyne-azide cycloaddition reaction, the divalent residue of the reactive group (e.g., RL) comprises a triazole ring or fused cyclic group comprising a triazole ring. In certain embodiments, when a conjugate is formed through a strain-promoted [3+2]alkyne-azide cycloaddition (SPAAC) reaction, the divalent residue of the reactive group (e.g., RL) is

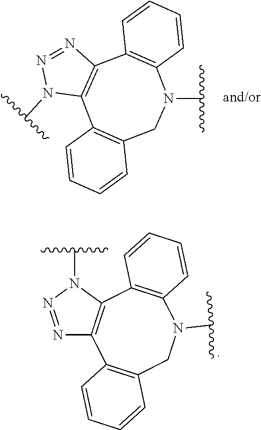 [SMILES: *N1Cc2ccccc2-c2c(nnn2*)-c2ccccc21 ; *N1Cc2ccccc2-c2nnn(*)c2-c2ccccc21]

If a conjugate of Formula (I), (Ia), (Ib), (Ic), (Ib-1), (Ib-2), (Ic-1), or (Ic-2) is formed by a [3+2] alkyne-azide cycloaddition, the conjugate encompasses both regioisomers. In certain embodiments, a conjugate of Formula (I), (Ia), (Ib), (Ic), (Ib-1), (Ib-2), (Ic-1), or (Ic-2) is a mixture of regioisomers formed from a [3+2] alkyne-azide cycloaddition.

### Inverse Electron Demand Ligation Reaction

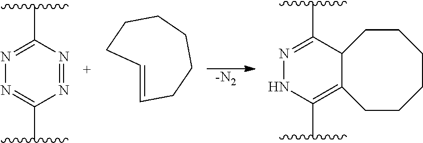

Advantageously, compounds comprising a terminal tetrazine or strained alkene group facilitate selective and efficient reactions with a second compound comprising a strained alkene or tetrazine group. It is believed that the tetrazine and strained alkene react in an inverse-demand Diels-Alder reaction followed by a retro-Diels-Alder reaction which links compounds comprising a terminal tetrazine or strained alkene group to the second compound. The reaction is believed to be specific, with little to no cross-reactivity with functional groups within biomolecules. The reaction may be carried out under mild conditions, for example, at room temperature and without a catalyst. This reaction between a tetrazine and a strained alkene is generally known to those in the art as a tetrazine ligation reaction.

In certain embodiments, when a conjugate is formed through a tetrazine inverse electron demand Diels-Alder ligation reaction, the divalent residue of the reactive group (e.g., RL) comprises a fused bicyclic ring having at least two adjacent nitrogen atoms in the ring. In certain embodiments, when a conjugate is formed through a tetrazine inverse electron demand Diels-Alder ligation reaction, the divalent residue of the reactive group (e.g., RL) is

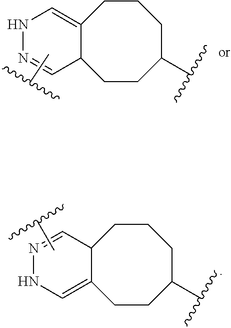

If a conjugate of Formula (I), (Ia), (Ib), (Ic), (Ib-1), (Ib-2), (Ic-1), or (Ic-2) is formed by an inverse electron demand ligation reaction, the conjugate encompasses both regioisomers. In certain embodiments, a conjugate of Formula (I), (Ia), (Ib), (Ic), (Ib-1), (Ib-2), (Ic-1), or (Ic-2) is a mixture of regioisomers formed from an inverse electron demand ligation reaction.

### Thiol Reactions

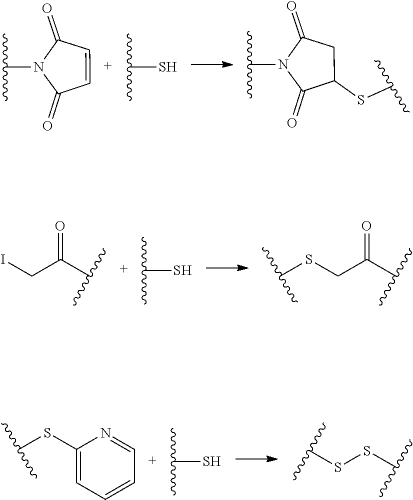

Advantageously, compounds comprising a terminal thiol group or suitable electrophilic or disulfide-forming group facilitate selective and efficient reactions with a second compound comprising a complementary electrophilic or disulfide-forming group or thiol group. These reactions are believed to be selective with little to no cross-reactivity with functional groups within biomolecules. In some embodiments, the thiol reaction does not include reaction of a maleimide group.

In certain embodiments, when a conjugate is formed through a thiol-maleimide reaction, the divalent residue of the reactive group comprises

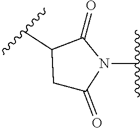 [SMILES: *C1CC(=O)N(*)C1=O]

and a sulfur linkage. In certain embodiments, when a conjugate is formed through a thiol-maleimide reaction,

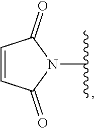 [SMILES: *N1C(=O)C=CC1=O]

the divalent residue of the reactive group (e.g., RL) is

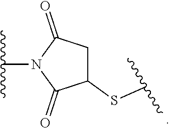 [SMILES: *SC1CC(=O)N(*)C1=O]

In certain embodiments, when a conjugate is formed through a thiol-maleimide reaction,

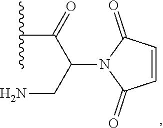 [SMILES: *C(=O)C(CN)N1C(=O)C=CC1=O]

the divalent residue of the reactive group (e.g., RL) is

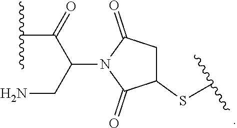 [SMILES: *SC1CC(=O)N(C(CN)C(*)=O)C1=O]

In certain embodiments, a conjugate is formed through a thiol-N-hydroxysuccinimide reaction using the following group:

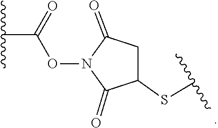 [SMILES: *SC1CC(=O)N(OC(*)=O)C1=O]

The reaction involved for formation of the conjugate comprises the following step:

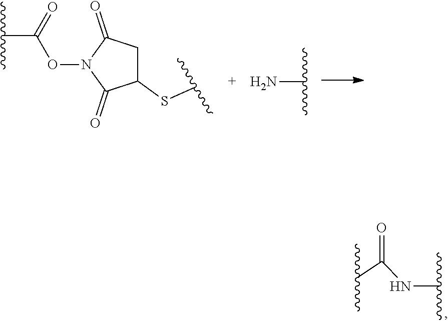

and the resulting divalent residue of the reactive group (e.g., RL) is

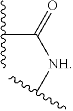 [SMILES: *NC(*)=O]

### Carbonyl-Oxyamine Reaction

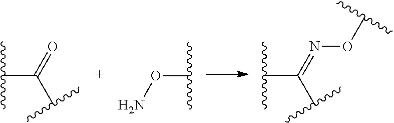

Advantageously, compounds comprising a terminal carbonyl or oxyamine group facilitate selective and efficient reactions with a second compound comprising an oxyamine or carbonyl group. It is believed that the carbonyl and oxyamine react to form an oxime linkage. The reaction is believed to be specific, with little to no cross-reactivity with functional groups within biomolecules.

In certain embodiments when a conjugate is formed through an oxime conjugation reaction, the divalent residue of the reactive group comprises a divalent residue of a non-natural or modified amino acid. In certain embodiments when a conjugate is formed through an oxime conjugation reaction, the divalent residue of the reactive group (e.g., RL) is

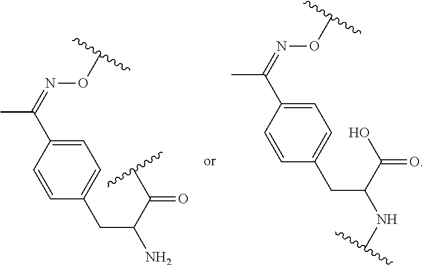 [SMILES: *O/N=C(/C)c1ccc(CC(N)C(*)=O)cc1 ; *NC(Cc1ccc(/C(C)=N\O*)cc1)C(=O)O]

In certain embodiments when a conjugate is formed through an oxime conjugation reaction, the divalent residue of the reactive group comprises an oxime linkage. In certain embodiments when a conjugate is formed through an oxime conjugation reaction, the divalent residue of the reactive group (e.g., RL) is

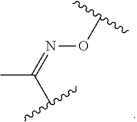 [SMILES: *O/N=C(\*)C]

### Other Reactions

Other suitable conjugation reactions are described in the literature. See, for example, Lang, K. and Chin, J. 2014, Bioorthogonal Reactions for Labeling Proteins, ACS Chem Biol 9, 16-20; Paterson, D. M. et al. 2014, Finding the Right (Bioorthogonal) Chemistry, ACS Chem Biol 9, 592-605; King, M. and Wagner, A. 2014, Developments in the Field of Bioorthogonal Bond Forming Reactions—Past and Present Trends, Bioconjugate Chem., 2014, 25 (5), pp 825-839; and Ramil, C. P. and Lin, Q., 2013, Bioorthogonal chemistry: strategies and recent developments, Chem Commun 49, 11007-11022.

### Vectors, Host Cells, and Recombinant Methods

The invention also provides isolated nucleic acids encoding antibodies, vectors and host cells comprising the nucleic acids, and recombinant techniques for the production of the antibodies.

The antibodies can be prepared according to techniques apparent to the person of skill. In certain embodiments, the antibodies are prepared from isolated nucleic acids encoding antibodies, vectors, and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. lichenformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for antibody-encoding vectors. Saccharomyces cerevisiae, or common baker's yeast, is a commonly used lower eukaryotic host microorganism. However, a number of other genera, species, and strains are available and useful, such as Spodoptera frugiperda (e.g., SF9), Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low. The antibodies produced in a cell-free system may be aglycosylated depending on the source of the cells.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxyapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### Pharmaceutical Compositions and Methods of Administration

(A) In one embodiment, a pharmaceutical composition comprising an antibody conjugate of any one of the embodiments described herein and a pharmaceutically acceptable carrier, excipient, or diluent is provided.

(B) In one embodiment, the pharmaceutical composition is according to embodiment (A) and is a pharmaceutical composition for treating abnormal cellular proliferation, wherein the abnormal cellular proliferation is cancer.

(C) In one embodiment, a method of treating abnormal cellular proliferation in a subject in need thereof is provided comprising administering an antibody conjugate of any one of the embodiments described herein or a pharmaceutical composition of embodiment (B), wherein the abnormal cellular proliferation is cancer.

(D) In one embodiment, the method is of embodiment (C) wherein the cancer is selected from lymphoma, head and neck squamous cell carcinoma, urothelial cancer, lung cancer, breast cancer, prostate cancer, SCLC, bladder cancer, and melanoma.

(E) In one embodiment, the use of an antibody conjugate of any one of the embodiments described herein or a pharmaceutical composition of embodiment (A) or (B) is provided for the treatment of abnormal cellular proliferation in a subject in need thereof, wherein the abnormal cellular proliferation is cancer.

(F) In one embodiment, the use of an antibody conjugate of any one of the embodiments described herein or a pharmaceutical composition of embodiment (A) or (B) is provided for the manufacture of a medicament for the treatment of abnormal cellular proliferation in a subject in need thereof, wherein the abnormal cellular proliferation is cancer.

The antibody conjugates provided herein can be formulated into pharmaceutical compositions using methods available in the art and those disclosed herein. Any of the antibody conjugates provided herein can be provided in the appropriate pharmaceutical composition and be administered by a suitable route of administration.

The methods provided herein encompass administering pharmaceutical compositions comprising at least one antibody conjugate provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions or antibody conjugates provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition, antibody, or antibody conjugate provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibody conjugates.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6^{th} Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6^{th} Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody conjugate, since, in some embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody or antibody-conjugate will decompose. Such excipients, which are referred to herein as “stabilizers,” include, but are not limited to, antioxidants such as ascorbic acid, pH buffers, or salt buffers.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody conjugate or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody conjugate per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram).

In certain embodiment, the dosage of the antibody conjugate provided herein, based on weight of the antibody conjugate, administered to prevent, treat, manage, or ameliorate a disorder, or one or more symptoms thereof in a subject is between 0.1 mg/kg and 15 mg/kg of the subject's body weight. In some embodiments, the dosage of the composition administered to a subject is between 0.1 mg and 200 mg.

The dose can be administered according to a suitable schedule. It may be necessary to use dosages of the antibody or antibody conjugate outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same.

In certain embodiments, treatment or prevention can be initiated with one or more loading doses of an antibody or antibody conjugate or composition provided herein followed by one or more maintenance doses.

In certain embodiments, a dose of an antibody conjugate or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

### Therapeutic Applications

For therapeutic applications, the antibody conjugates provided herein can be administered to a mammal, generally a human, in a pharmaceutically acceptable dosage form such as those known in the art and those discussed above. For example, the antibody conjugates may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibody conjugates also are suitably administered by peritumoral, intralesional, or perilesional routes, to exert local as well as systemic therapeutic effects. The intraperitoneal route may be particularly useful, for example, in the treatment of ovarian tumors. In a certain embodiment, the antibody conjugates may be administered to a human intravenously.

The antibodies provided herein may be useful for the treatment of any disease or condition involving TROP2. In some embodiments, the disease or condition is a disease or condition that can be diagnosed by overexpression of TROP2. In some embodiments, the disease or condition is a disease or condition that can benefit from treatment with an anti-TROP2 antibody. In some embodiments, the disease or condition is abnormal cellular proliferation, for example cancer.

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas. Additional examples of solid tumors include, but are not limited to, squamous cell carcinoma, colon cancer, breast cancer, prostate cancer, lung cancer, liver cancer, pancreatic cancer, and melanoma. In one embodiment, the solid tumor is an advanced solid tumor.

In some embodiments, the term “cancer” includes, but is not limited to, the following cancers: Cancer to be treated by the present invention is not especially limited, and examples thereof include various peritoneal metastatic cancers, gastric cancer, lung cancer, blood cancers such as acute lymphoblastic leukemia, acute myelogenous leukemia, Hodgkin's lymphoma, non-Hodgkin's lymphoma, B cell lymphoma, multiple myeloma and T cell lymphoma, solid cancers such as myelodysplastic syndromes, adenocarcinoma, squamous cell carcinoma, adenosquamous carcinoma, undifferentiated carcinoma, large cell carcinoma, non-small cell lung cancer, small cell lung cancer, mesothelioma, skin cancer, skin T cell lymphoma, breast cancer, prostate cancer, bladder cancer, vaginal cancer, cervix cancer, head and neck cancer, uterine cancer, cervical cancer, liver cancer, gallbladder cancer, bile duct cancer, kidney cancer, pancreatic cancer, colon cancer, colorectal cancer, rectal cancer, small intestine cancer, gastric cancer, esophageal cancer, testicular cancer, ovarian cancer and brain tumor, cancers of bone tissues, cartilage tissues, adipose tissues, muscle tissues, vascular tissues and blood-forming tissues, sarcomas such as chondrosarcoma, Ewing's sarcoma, malignant hemangioendothelioma, malignant schwannoma, osteosarcoma and soft tissue sarcoma, and blastomas such as glioblastoma, glioblastoma multiforme, hepatoblastoma, medulloblastoma, nephroblastoma, neuroblastoma, pancreatoblastoma, pleuropulmonary blastoma and retinoblastoma. In one embodiment, cancer to be treated by the present invention is colorectal cancer, non-small cell lung cancer, small cell lung cancer, bladder cancer, ovarian cancer, breast cancer or prostate cancer. In one embodiment, cancer to be treated by the present invention is cancer in which TROP2 is highly expressed as compared to a normal tissue. Cancer to be treated by the present invention is preferably cancer in which TROP2 is highly expressed as compared to a normal tissue, or cancer selected from the group consisting of colorectal cancer, rectal cancer, lung cancer, non-small cell lung cancer, small cell lung cancer, bladder cancer, ovarian cancer, breast cancer and prostate cancer. In certain embodiments, the cancer is selected from acute myeloid leukemia, breast cancer, colorectal cancer, glioma, head and neck squamous cell carcinoma, lung cancer, including non-small cell lung cancer, head and neck cancer, lymphoma, including a malignant lymphoma, melanoma, nasopharyngeal carcinoma, ovary cancer, pancreatic cancer, prostate cancer, urothelial cancer, and tongue squamous cell carcinoma. In certain embodiments, the cancer is selected from lymphoma, head and neck squamous cell carcinoma, urothelial cancer, lung cancer, breast cancer, prostate cancer, SCLC, bladder cancer, and melanoma.

Also provided herein is a method of inducing an immune response in a subject in need thereof comprising administering an antibody conjugate or pharmaceutical composition described herein.

In certain embodiments, the antibody conjugates provided herein treat the disease, for example cancer, by activating anti-tumor immunity or protective immunity. In certain embodiments, tumor cells pre-treated with an antibody or antibody conjugate provided herein undergo immunogenic cell damage, which can, in turn mount protective immunity in vivo.

### Diagnostic Applications

In some embodiments, the antibody conjugates provided herein are used in diagnostic applications. These assays may be useful, for example, in making a diagnosis and/or prognosis for a disease, such as cancer.

In some diagnostic and prognostic applications, the antibody conjugate may be labeled with a detectable moiety. Suitable detectable moieties include, but are not limited to radioisotopes, fluorescent labels, and enzyme-substrate labels. In another embodiment, the antibody conjugate need not be labeled, and the presence of the antibody conjugate can be detected using a labeled antibody which specifically binds to the antibody conjugate.

### Affinity Purification Reagents

The antibody conjugates provided herein may be used as affinity purification agents. In this process, the antibody conjugates may be immobilized on a solid phase such a resin or filter paper, using methods well known in the art. The immobilized antibodies or antibody conjugate is contacted with a sample containing the antigen (or fragment thereof) to be purified, and thereafter the support is washed with a suitable solvent that will remove substantially all the material in the sample except the protein of interest, which is bound to the immobilized antibody. Finally, the support is washed with another suitable solvent, such as glycine buffer, pH 5.0 that will release the protein from the antibody.

### Kits

In some embodiments, an antibody conjugate provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the antibody conjugate. In some embodiments, the antibody conjugate is provided in the form of a pharmaceutical composition.

### EXAMPLES

Proton nuclear magnetic resonance (NMR) spectra were obtained on Bruker Ascend™ 500 MHz spectrometer. NMR spectra are reported as follows: chemical shift δ (ppm), multiplicity, coupling constant J (Hz), and integration. The abbreviations s=singlet, d=doublet, t=triplet, q=quartet, m=multiplet and br=broad are used throughout. Mass spectral data were measured using the following systems: Agilent Technologies 1290 series, Binary Pump, Diode Array Detector. Data was acquired using agilent software and purity characterized by UV wavelength 220 nm, evaporative light scattering detection (ELSD) and electrospray positive ion (ESI) (column: Agilent Poroshell 120 EC-C18, 2.7 μm, 4.6×50 mm). Solvents used: acetonitrile/water, containing 0.1% formic acid; flow rate 1 mL/min.

For all of the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

As used herein, the symbols and conventions used in these processes, schemes and examples, regardless of whether a particular abbreviation is specifically defined, are consistent with those used in the contemporary scientific literature, for example, the Journal of Biological Chemistry and/or the Journal of the American Chemical Society.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Example 1. Synthesis of STING and Toxin Linker Payloads

### Example 1A. Synthesis of Select Intermediates for Synthesizing Select STING Agonists Described Herein

Common intermediates of the STING amidobenzimidazole class of agonists examples were prepared according to the following schemes and used to make the select STING agonists compounds described herein.

### Synthesis 1A-1. Intermediate I-1-5 and I-1-10

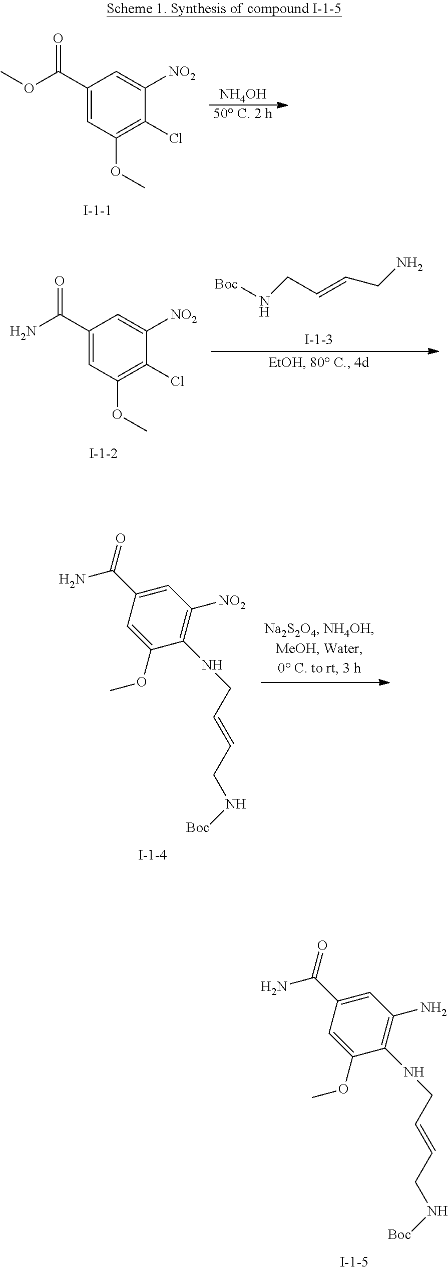

4-Chloro-3-methoxy-5-nitrobenzamide (I-1-2): Methyl 4-chloro-3-methoxy-5-nitrobenzoate (1) (40 g, 163 mmol) was stirred in NH₄OH (600 mL) at room temperature for 24 h. The reaction temperature was then increased to 50° C. for 2 h. An additional 120 mL of NH₄OH was added to the vessel. After an additional 2 h of stirring at 50° C., the reaction was cooled to room temperature. The solid was filtered and rinsed with cold water. The solid was dried under vacuum to give compound I-1-2 (29 g) as a tan solid. LC-MS [ESI]: Calculated for C₈H₇ClN₂O₄ [M+H⁺]: 231.01, Found: 231.1.

tert-Butyl (E)-(4-((4-carbamoyl-2-methoxy-6-nitrophenyl)amino)but-2-en-1-yl)carbamate (I-1-4): To a suspension of compound I-1-2 (30 g, 130 mmol) in EtOH (400 mL) was added (E)-tert-butyl (4-aminobut-2-en-1-yl) carbamate (I-1-3) (28 g, 150 mmol) and N,N-diisopropylethylamine (hereinafter called DIPEA, 65 mL, 374 mmol). The reaction was stirred at 80° C. for 4 days and allowed to cool to room temperature. The resulting orange color solid was collected by filtration and washed with EtOH (100 mL), dried in vacuum to afford compound I-1-4 as an orange solid (27 g); LC-MS [ESI]: Calculated for C₁₇H₂₄N₄O₆ [M+H⁺]: 381.17, Found: 381.2.

tert-Butyl (E)-(4-((2-amino-4-carbamoyl-6-methoxyphenyl)amino)but-2-en-1-yl)carbamate (I-1-5): To compound 4 (27 g, 71 mmol) was added methanol (480 mL). This mixture was cooled down to 0° C. After 20 minutes of stirring at 0° C., ammonium hydroxide solution (29% wt, 100 mL) was added followed by sodium hydrosulfite (75 g, 431 mmol) as a solution in water (194 mL). The flask was removed from the ice bath and stirred at room temperature. After 3 h of stirring at room temperature, water (800 mL) was added until a clear solution was obtained. The methanol was evaporated using reduced pressure. The white solid that formed during evaporation was filtered off, washed with water twice (300 mL each), and dried under vacuum to give compound I-1-5 as a white solid (17 g). LC-MS [ESI]: Calculated for C₁₇H₂₆N₄O₄ [M+H⁺]: 351.2, Found: 351.2

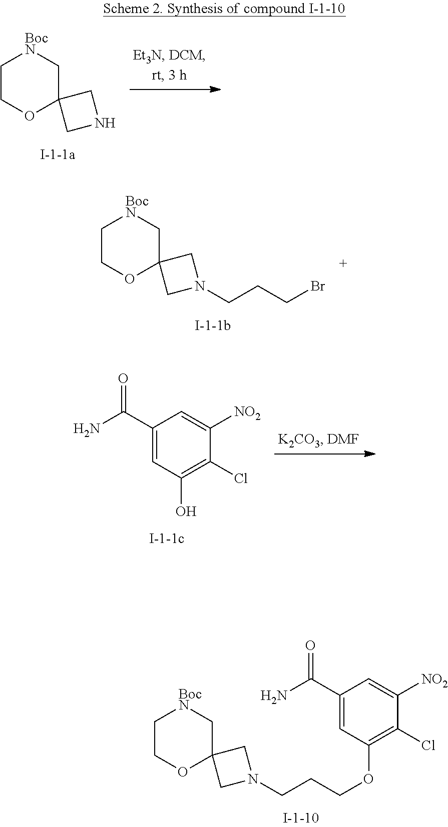 [SMILES: CC(C)(C)OC(=O)N1CCOC2(CNC2)C1 ; CC(C)(C)OC(=O)N1CCOC2(CN(CCCBr)C2)C1 ; NC(=O)c1cc(O)c(Cl)c([N+](=O)[O-])c1 ; CC(C)(C)OC(=O)N1CCOC2(CN(CCCOc3cc(C(N)=O)cc([N+](=O)[O-])c3Cl)C2)C1]

tert-Butyl 2-(3-bromopropyl)-5-oxa-2,8-diazaspiro[3.5]nonane-8-carboxylate (I-1-1b): To a stirred solution of tert-butyl 5-oxa-2,8-diazaspiro[3.5]nonane-8-carboxylate 1a (5 g, 21.9 mmol) in DCM (45 mL) at room temperature was added 1,3-dibromopropane (2.4 mL, 24.1 mmol). The reaction mixture was stirred at rt for 1 h. To this was added triethylamine (3 mL, 21.9 mmol) and the resulting suspension was stirred at rt for 3 h. To this another 0.5 eq of 1,3 dibromo propane was added and the reaction was continued for another 2 h. After which, the reaction mixture was concentrated under reduced pressure and residue was suspended in EtOAc (25 mL). Solids were filtered and washed with EtOAc (2×10 mL). The combined filtrate was concentrated under reduced pressure and the crude material was purified by column chromatography over silica gel (dry load, 40 g column, 0-20% MeOH/DCM as eluent) to afford tert-butyl 2-(3-bromopropyl)-5-oxa-2,8-diazaspiro[3.5]nonane-8-carboxylate I-1-1b (3.5 g) as colorless oil. ¹H NMR (400 MHz, CDCl₃) δ 3.61-3.56 (m, 2H), 3.55 (s, 2H), 3.45 (t, J=6.7 Hz, 2H), 3.41-3.32 (m, 4H), 2.89 (d, J=7.7 Hz, 2H), 2.63 (t, J=6.8 Hz, 2H), 1.92 (p, J=6.7 Hz, 2H), 1.49 (s, 9H).

tert-Butyl 2-[3-(5-carbamoyl-2-chloro-3-nitro-phenoxy)propyl]-5-oxa-2,8-diazaspiro[3.5]nonane-8-carboxylate (I-1-10): To a stirred suspension of 4-chloro-3-hydroxy-5-nitro-benzamide I-1-1c (3.2 g, 15.03 mmol), and K₂CO₃ (3.46 g, 25.05 mmol) in DMF (10 mL) at room temperature was added tert-butyl 2-(3-bromopropyl)-5-oxa-2,8-diazaspiro[3.5]nonane-8-carboxylate (I-1-1b, 3.5 g, 10 mmol) and the reaction mixture was stirred at 70° C. for 16 h. The reaction mixture was cooled to room temperature, quenched with ice cold water, and extracted with EtOAc (3×100 mL). The combined organic layer was washed with water (3×50 mL) followed by brine, dried over sodium sulfate, and concentrated under reduced pressure. The crude material was triturated with 50% EtOAc in heptane to afford tert-butyl 2-[3-(5-carbamoyl-2-chloro-3-nitro-phenoxy)propyl]-5-oxa-2,8-diazaspiro[3.5]nonane-8-carboxylate (I-1-10, 2.6 g, 5.3 mmol) as an off white solid. ¹H NMR (400 MHz, DMSO) δ 8.29 (s, 1H), 8.04 (d, J=1.7 Hz, 1H), 7.87 (d, J=1.9 Hz, 1H), 7.77 (s, 1H), 4.25 (t, J=6.2 Hz, 2H), 3.52-3.38 (m, 4H), 3.30-3.22 (m, 4H), 2.80-2.71 (m, 2H), 2.62 (t, J=6.8 Hz, 2H), 1.82 (p, J=6.6 Hz, 2H), 1.39 (s, 9H). ¹³C NMR (101 MHz, DMSO) δ 165.3, 155.5, 154.5, 149.2, 135.0, 116.9, 115.8, 115.7, 79.6, 70.9, 68.6, 62.3, 61.9, 55.8, 28.4, 27.3. LC-MS [ESI]: Calculated for C₂₁H₂₉ClN₄O₇ [M+H⁺]: 485.18, Found: 485.20;

### Synthesis 1A-2. Intermediate I-1-13

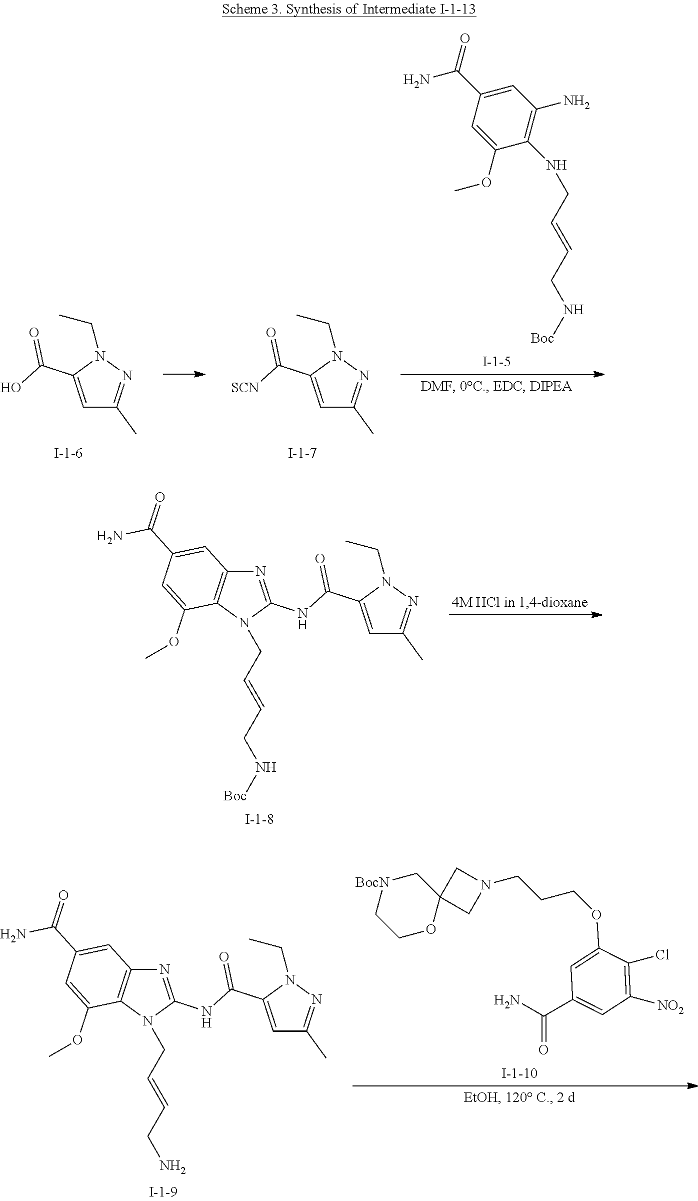

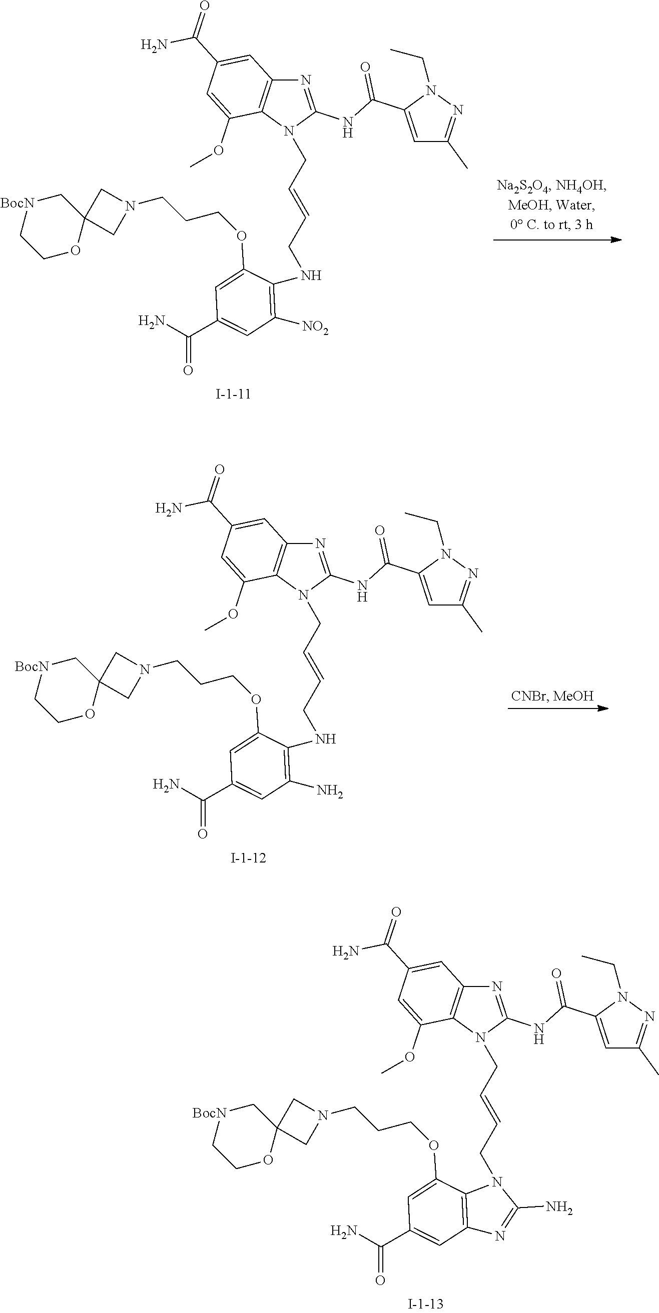 [SMILES: CCn1nc(C)cc1C(=O)Nc1nc2cc(C(N)=O)cc(OC)c2n1C/C=C/CNc1c(OCCCN2CC3(C2)CN(C(=O)OC(C)(C)C)CCO3)cc(C(N)=O)cc1[N+](=O)[O-] ; CCn1nc(C)cc1C(=O)Nc1nc2cc(C(N)=O)cc(OC)c2n1C/C=C/CNc1c(N)cc(C(N)=O)cc1OCCCN1CC2(C1)CN(C(=O)OC(C)(C)C)CCO2 ; CCn1nc(C)cc1C(=O)Nc1nc2cc(C(N)=O)cc(OC)c2n1C/C=C/Cn1c(N)nc2cc(C(N)=O)cc(OCCCN3CC4(C3)CN(C(=O)OC(C)(C)C)CCO4)c21]

1-Ethyl-3-methyl-1H-pyrazole-5-carbonyl isothiocyanate (I-1-7): To a 1 L round bottom flask was added 1-ethyl-3-methyl-1H-pyrazole-5-carboxylic acid (I-1-6, 10 g, 65 mmol) and DCM (200 mL). To this heterogeneous solution was added DMF (0.1 mL) followed by the slow addition of oxalyl chloride (7 mL, 82 mmol). After stirring for 1 h at room temperature, the volatiles were removed under vacuum and the crude was co-evaporated twice with dichloromethane (100 mL each). Then the crude residue was dissolved in anhydrous acetone (25 mL) and added to a solution of KSCN (8.6 g, 89 mmol) in anhydrous acetone (150 mL) at 0° C. The mixture was stirred for 20 min. At this time, hexane (100 mL) was added to the reaction mixture and the crude heterogeneous solution was concentrated under reduced pressure to one third of the volume. The process of hexane addition and concentration was repeated twice (200 mL of hexane each). After the last concentration, hexane (200 mL) was added and the solid was removed by filtration, rinsing with hexane (100 mL). The resulting clear light yellow filtrate was concentrated and purified by chromatography (330 g silica column; eluting with 0-20% EtOAc/hexane). The desired product eluted at 7% EtOAc/hexane. The fractions were combined and concentrated yielding compound I-1-7 (10 g) as a clear colorless liquid.

tert-Butyl (E)-(4-(5-carbamoyl-2-(1-ethyl-3-methyl-1H-pyrazole-5-carboxamido)-7-methoxy-1H-benzo[d]imidazol-1-yl)but-2-en-1-yl)carbamate (I-1-8): To a solution of compound I-1-5 (15.8 g, 45.2 mmol) in anhydrous DMF (145 mL) at 0° C. was added a solution of compound I-1-7 (7.9 g, 40.7 mmol) in dioxane (40 mL). After 20 minutes of stirring at 0° C., the formation of the intermediate thiourea was complete. Then EDC (15.87 g, 83 mmol) and DIPEA (28.9 mL) were subsequently added. The reaction was warmed to room temperature and stirred overnight. To the heterogenous reaction mixture was added a mixture of 250 mL of saturated aqueous ammonium chloride and 750 mL of water. This heterogenous mixture was stirred for 1 h at room temperature. The solid was filtered off, rinsed twice with water (200 mL each), and dried to give compound I-1-8 as a white solid (20 g). LC-MS [ESI]: Calculated for C₂₅H₃₃N₇O₅ [M+H⁺]: 512.25, Found: 512.3.

(E)-1-(4-Aminobut-2-en-1-yl)-2-(1-ethyl-3-methyl-1H-pyrazole-5-carboxamido)-7-methoxy-1H-benzo[d]imidazole-5-carboxamide (I-1-9): To a suspension of compound I-1-8 (10 g, 19.5 mmol) in 80 mL of methanol was added HCl (114 mL) as a 4M solution in 1,4-dioxane. During the HCl addition the solution went from heterogenous to homogenous with a clear yellow color. The reaction was stirred at room temperature overnight. The white solid was filtered off, rinsed with ether (200 mL), and dried in vacuum to give compound I-1-9 as HCl salt (6 g). LC-MS [ESI]: Calculated for C₂₀H₂₅N₇O₃ [M+H⁺]: 412.2, Found: 412.3.

(E)-2-(3-(5-Carbamoyl-2-((4-(5-carbamoyl-2-(1-ethyl-3-methyl-1H-pyrazole-5-carboxamido)-7-methoxy-1H-benzo[d]imidazol-1-yl)but-2-en-1-yl)amino)-3-nitrophenoxy)propyl)-5-oxa-2,8-diazaspiro[3.5]nonane-8-carboxylate (I-1-11): To a suspension of compound I-1-9 (3 g, 5.7 mmol) in EtOH (40 mL) in a pressure vessel was added compound I-1-10 (2.5 g, 5.2 mmol) and DIPEA (5 mL). The reaction was stirred at 120° C. for 2 days and allowed to cool to room temperature. The resulting precipitate was collected by filtration, washed with EtOH (20 mL) and dried under vacuum to give compound I-1-11 as a tan solid (2.3 g). LC-MS [ESI]: Calculated for C₄₁H₅₃N₁₁O₁₀ [M+H⁺]: 860.4, found: 860.4.

tert-Butyl (E)-2-(3-(3-amino-5-carbamoyl-2-((4-(5-carbamoyl-2-(1-ethyl-3-methyl-1H-pyrazole-5-carboxamido)-7-methoxy-1H-benzo[d]imidazol-1-yl)but-2-en-1-yl)amino)phenoxy)propyl)-5-oxa-2,8-diazaspiro[3.5]nonane-8-carboxylate (I-1-12): To compound I-1-11 (2.3 g, 2.7 mmol) was added methanol (50 mL). This mixture was cooled down to 0° C. After 20 minutes of stirring at 0° C., ammonium hydroxide solution (29% wt, 10 mL) was added followed by sodium hydrosulfite (3.2 g, 18.4 mmol) as a solution in water (15 mL). The flask was removed from the ice bath and stirred at room temperature. After 3 h of stirring at room temperature, water (100 mL) was added until a clear solution was obtained. The methanol was evaporated under reduced pressure. The sticky solid that formed during evaporation was collected by decantation, washed with water twice (100 mL each), and dried under vacuum to give compound I-1-12 as a sticky solid (1.9 g). LC-MS [ESI]: Calculated for C₄₁H₅₅N₁₁O₈[M+H⁺]: 830.42, Found: 831.5.

tert-Butyl (E)-2-(3-((2-amino-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-7-yl)oxy)propyl)-5-oxa-2,8-diazaspiro[3.5]nonane-8-carboxylate (I-1-13): To a solution of compound I-1-12 (1.7 g, 2.1 mmol) in MeOH (20 mL) was added cyanogen bromide (446 mg, 4.2 mmol) at room temperature. After 5 h, the reaction was concentrated and purified by reverse phase preparative HPLC (Phenomenex Gemini NX 5 m, C18, 110 Å, 150×50 mm; Mobile phase: A) water (0.1% TFA), B) acetonitrile; Gradient: 5-45% B over 20 min, flow 50 mL/min) to give compound I-1-13 (302 mg) as TFA salt. LC-MS [ESI]: Calculated for C₄₂H₅₄N₁₂O₈ [M+H⁺]: 855.42, Found: 855.5.

### Synthesis 1A-3. Intermediate I-2

Common Intermediate I-2 was synthesized as shown in Scheme 4 below.

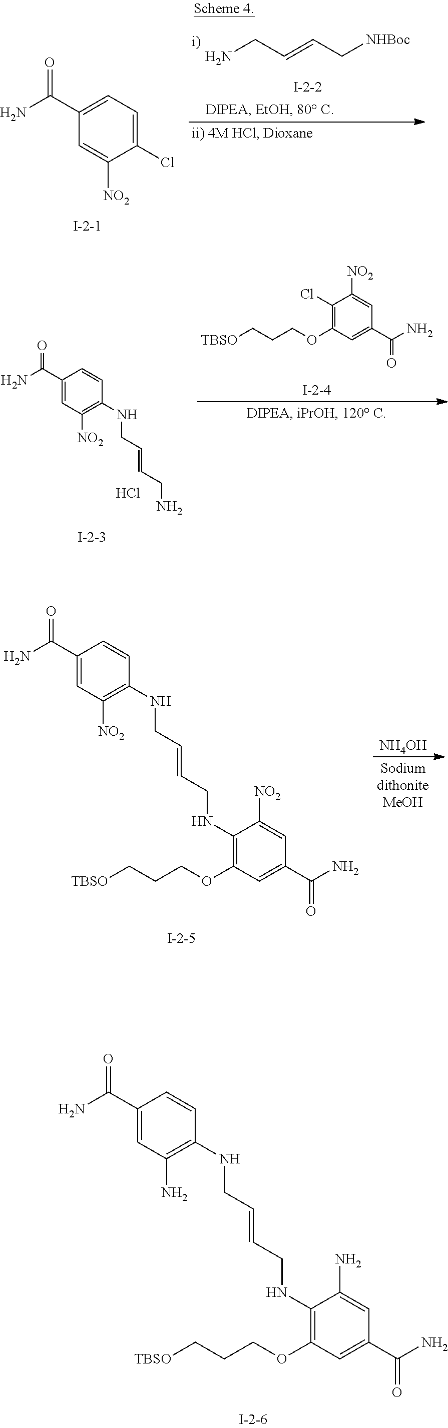

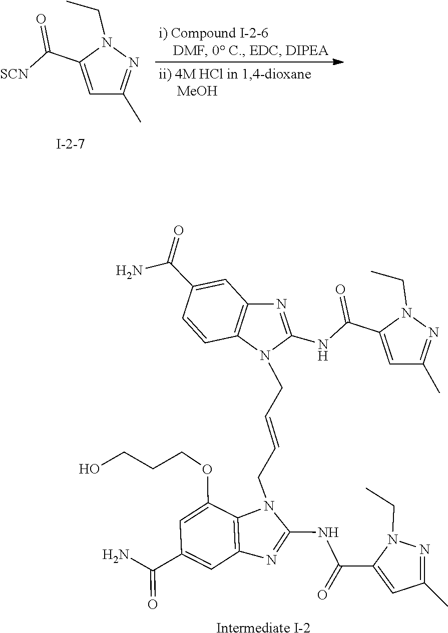

### tert-butyl (E)-(4-((4-carbamoyl-2-nitrophenyl)amino)but-2-en-1-yl)carbamate (I-2-2-1)

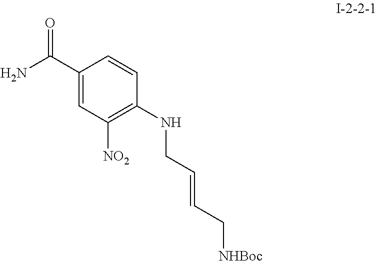 [SMILES: CC(C)(C)OC(=O)NC/C=C/CNc1ccc(C(N)=O)cc1[N+](=O)[O-]]

To a suspension of compound I-2-1 (40 g, 200 mmol) in EtOH (500 mL) was added (E)-tert-butyl (4-aminobut-2-en-1-yl) carbamate (I-2-2) (44.6 g, 240 mmol) and DIPEA (105 mL, 604 mmol). The reaction was stirred at 80° C. for 4 days and allowed to cool to room temperature. The resulting orange solid was collected by filtration and washed with EtOH (100 mL) to give compound I-2-2-1 as an orange solid (70 g, >95% pure) LC-MS [ESI]: Calculated for C₁₆H₂₃N₄O₅ [M+H⁺]: 351.16, Found: 351.36.

### (E)-4-((4-aminobut-2-en-1-yl)amino)-3-nitrobenzamide (I-2-3)

To a suspension of compound I-2-2-1 (68 g, 194 mmol) in MeOH (160 mL) was added HCl (4M in 1,4 dioxane, 230 mL). The reaction mixture was stirred at room temperature for 2 h. Then the mixture was evaporated under reduced pressure and dried under vacuum to give the compound I-2-3 (64 g, >95% pure) as a HCl salt. LC-MS [ESI]: Calculated for C₁₁H₁₅N₄O₃ [M+H⁺]: 251.11, Found: 251.23.

### (E)-3-(3-((tert-butyldimethylsilyl)oxy)propoxy)-4-((4-((4-carbamoyl-2-nitrophenyl)amino)but-2-en-1-yl)amino)-5-nitrobenzamide (I-2-5)

To a suspension of compound I-2-3 (HCl salt, 10 g, 31 mmol) in anhydrous iPrOH (50 mL) in a pressure vessel was added compound I-2-4 (12 g, 31 mmol) and DIPEA (27 mL). The reaction was stirred at 120° C. for 7 days and allowed to cool to room temperature. The solvent was evaporated under vacuum, and the resulting residue was dissolved in acetonitrile (250 mL) at 60° C. The solution was cooled with an ice bath, and the resulting precipitate was collected by filtration, washed with ice cold acetonitrile (20 mL) and dried under vacuum to give crude compound I-2-5 as a red solid (about 80% pure, 11 g). Repeated on the same scale 3 more times due to a limited volume of the available pressure vessel. LC-MS [ESI]: Calculated for C₂₇H₃₉N₆O₈Si [M+H⁺]: 603.25, Found: 603.48.

### (E)-3-amino-4-((4-((2-amino-4-carbamoylphenyl)amino)but-2-en-1-yl)amino)-5-(3-((tert-butyldimethylsilyl)oxy)propoxy)benzamide (I-2-6)

To crude compound I-2-5 (˜80% pure, 43 g, 71.4 mmol) was added methanol (400 mL) to make a suspension. This mixture was cooled down to 0° C. After 20 minutes of stirring at 0° C., ammonium hydroxide solution (29% wt, 150 mL) was added followed by water (250 mL) and sodium hydrosulfite (74 g, 425 mmol). The flask was removed from the ice bath and stirred at room temperature. After 2 h of stirring at room temperature, the insoluble material was filtered off and the filtrate was extracted with DCM (2×800 mL). DCM solution was washed with brine, then dried over Na₂SO₄, filtered, and evaporated under reduced pressure to give a dark residue. This residue was dissolved in DCM/MeOH mixture (9:1, 20 mL) and purified by silicagel column (330 g silicagel, DCM/MeOH (10% to 20%) to give compound I-2-6 as a tan solid (15 g, yield 38%, >90% pure). LC-MS [ESI]: Calculated for C₂₇H₄₃N₆O₄Si [M+H⁺]: 543.31, Found: 543.4.

### Compound I-2-7

To a 1 L round bottom flask was added 1-ethyl-3-methyl-1H-pyrazole-5-carboxylic acid (20 g, 130 mmol) and DCM (400 mL). To this heterogeneous solution was added DMF (0.2 mL) followed by the slow addition of oxalyl chloride (14 mL, 164 mmol). After stirring for 1 h at room temperature, the volatiles were removed under vacuum and the crude was co-evaporated twice with dichloromethane (200 mL each). Then the crude residue was dissolved in anhydrous acetone (50 mL) and added to a solution of KSCN (17.2 g, 178 mmol) in anhydrous acetone (300 mL) at 0° C. The mixture was stirred for 20 min. At this time, hexane (200 mL) was added to the reaction mixture and the crude heterogeneous solution was concentrated in vacuo to one third of the volume. The process of hexane addition and concentration was repeated twice (300 mL of hexane each). After the last concentration, hexane (400 mL) was added and the solid was removed by filtration, rinsing with hexane (100 mL). The resulting clear brown filtrate was concentrated and purified by chromatography (330 g silica column; eluting with 0-20% EtOAc/hexane). The desired product elutes at 7% EtOAc/hexane. The fractions were combined and concentrated yielding compound I-2-7 (20 g) as a clear colorless liquid. LC-MS [ESI]: Calculated for C₈H₁₀N₃OS [M+H⁺]: 196.06, Found: 196.1.

### (E)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-7-(3-hydroxypropoxy)-1H-benzo[d]imidazole-5-carboxamide (Intermediate I-2)

To a solution of compound I-2-6 (13 g, 24 mmol) in anhydrous DMF (60 mL) at 0° C. was added a solution of compound I-2-7 (14 g, 72 mmol). After 3 h of stirring at room temperature, the formation of the intermediate thiourea was complete. Then EDC (18 g, 94 mmol) and DIPEA (17 mL) were sequentially added. The reaction was stirred at room temperature for 24 h. The reaction mixture was concentrated under reduced pressure to 50 mL to give mostly DMF solution of crude compound, LC-MS [ESI]: Calculated for C₄₃H₅₇N₁₂O₆Si [M+H⁺]: 865.42, Found: 865.6. To this crude solution was added acetonitrile (75 mL), water (75 mL) and TFA (8 mL). The resulting mixture was stirred for 16 h at room temperature. The solid was filtered off, rinsed twice with acetonitrile/water mixture (1:1, 120 mL each), and dried to give compound Intermediate I-2 as a tan solid (TFA salt, 11 g, >95% pure).

### Synthesis 1A-4. Intermediate I-4

Common Intermediate I-4 was synthesized as shown in Scheme 5 below.

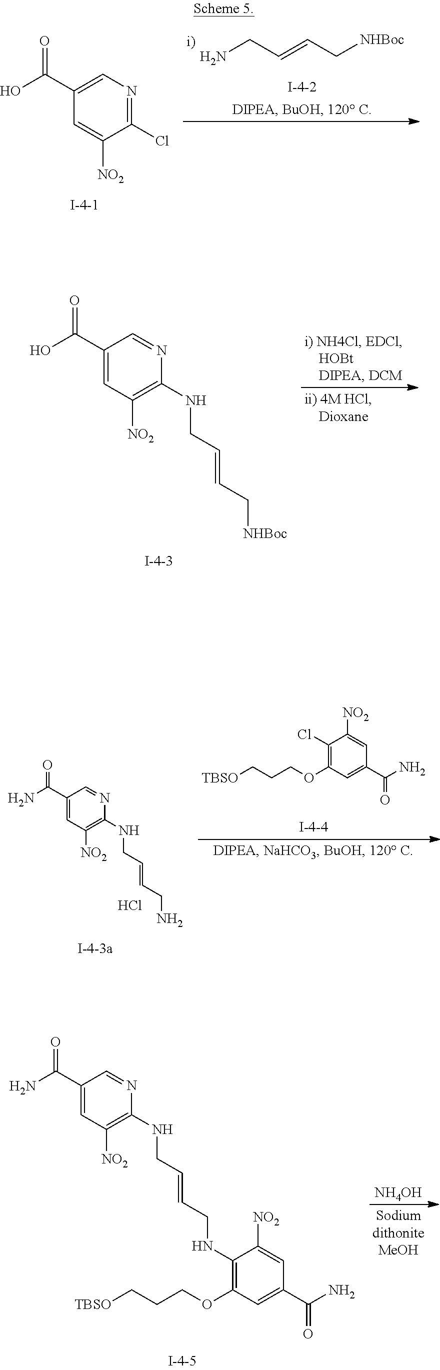

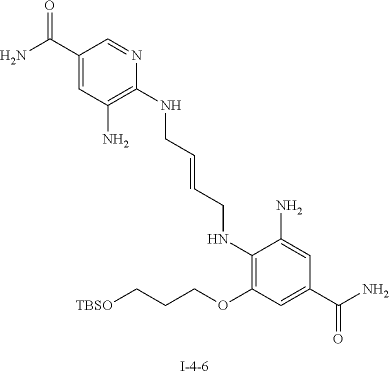 [SMILES: CC(C)(C)[Si](C)(C)OCCCOc1cc(C(N)=O)cc(N)c1NC/C=C/CNc1ncc(C(N)=O)cc1N]

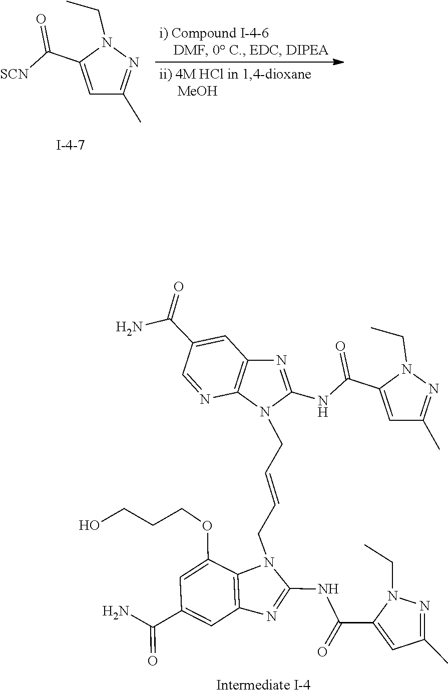

4-Chloro-3-[3-(1-methyl-1-trimethylsilyl-ethoxy)propoxy]-5-nitro-benzamide (I-4-4): In a sealed tube flask, was taken 4-chloro-3-hydroxy-5-nitro-benzamide (18.5 g, 85 mmol) and K₂CO₃ (23.6 g, 170 mmol) in DMF (150 mL). To this was added [1-(3-bromopropoxy)-1-methyl-ethyl]-trimethyl-silane (28.2 g, 110.5 mmol). The vessel was capped, and the resulting suspension was heated to 100° C. for 2 h followed by at rt for 16 h. Reaction mixture poured onto ice cold water and filtered. Solids were washed with water followed by hexanes and dried under reduced pressure to afford 4-chloro-3-[3-(1-methyl-1-trimethylsilyl-ethoxy)propoxy]-5-nitro-benzamide I-4-4 (28 g, 84%) as light yellow solid. ¹H NMR (400 MHz, DMSO) δ 8.30 (s, 1H), 8.05 (d, J=1.7 Hz, 1H), 7.89 (d, J=1.8 Hz, 1H), 7.78 (s, 1H), 4.30 (t, J=5.9 Hz, 2H), 3.80 (t, J=6.1 Hz, 2H), 1.98 (p, J=6.0 Hz, 2H), 0.84 (s, 10H), 0.06 (s, 6H). LC-MS [ESI]: Calculated for C₁₆H₂₅ClN₂O₅Si [M+H⁺]: 389.13, Found: 389.20.

(E)-6-((4-((tert-butoxycarbonyl)amino)but-2-en-1-yl)amino)-5-nitronicotinic acid (I-4-3): To a stirred solution of 6-chloro-5-nitronicotinic acid (I-4-1, 100 g, 494 mmol) in ethanol (1 L) was added (E)-tert-butyl (4-aminobut-2-en-1-yl)carbamate (I-4-2, 92 g, 494 mmol) and DIPEA (128 g, 988 mmol). The mixture was heated to 85° C. and stirred for 16 hours. The reaction mixture was concentrated under reduced pressure. The compound was dissolved in DCM (1.5 L) and water (1.5 L), and adjusted pH to 6˜7 by adding aq. 1M HCl, concentrated to remove DCM, and the remainder was filtered and washed with water (100 mL) and dried under vacuum to afford compound I-4-3 as a yellow solid (78 g, 45%). LC-MS [ESI]: Calculated for C₁₅H₂₀N₄O₆ [M+H⁺]: 353.14, Found: 354.2.

(E)-6-((4-aminobut-2-en-1-yl)amino)-5-nitronicotinamide (I-4-3a): To a stirred solution of compound I-4-3, 78 g, 222 mmol) in DCM (780 mL) was added NH₄Cl (14.2 g, 266.4 mmol), DIPEA (100.2 g, 333 mmol), EDCI (63.6 g, 333 mmol and HOBT (45 g, 777 mmol). The reaction mixture was stirred at room temperature for 16 hours. The reaction mixture was filtered, washed with DCM (50 mL), and dried to afford compound Boc-protected I-4-3a as a yellow solid (63 g, 81%). To a stirred solution of Boc-protected compound I-4-3a (62 g, 176 mmol) in 1,4-dioxane (60 mL), 4M HCl 1,4-dioxane (60 mL) was added and the reaction mixture was stirred 16 h at room temperature. The mixture was concentrated under reduced pressure, the remaining was dissolved in water (60 mL) and adjusted pH=10 by adding saturated sodium carbonate solution and the mixture was stirred for 4 h, and filtered and dried to give compound I-4-3a (42 g) as a yellow solid. LC-MS [ESI]: Calculated for C₁₀H₁₃N₅O₃ [M+H⁺]: 252.1, Found: 252.1.

(E)-6-((4-((2-(3-((tert-butyldimethylsilyl)oxy)propoxy)-4-carbamoyl-6-nitrophenyl)amino)but-2-en-1-yl)amino)-5-nitronicotinamide (I-4-5): To a stirred solution of compound I-4-4 (30 g, 0.077 mol), compound I-4-3a (29 g, 0.116 mol) and Na₂CO₃ (16.4 g, 0.154 mol) in n-butanol (750 mL) was added and the mixture was heated to 120° C. for 16 h. The mixture was cooled to room temperature, DCM (750 mL) was added, the reaction was stirred for 30 min, and filtered to remove solids. The filtrate was concentrated under reduced pressure, and purified by silica gel column chromatography (DCM:MeOH=30:1) to give compound I-4-5 (19 g) as a red solid. LC-MS [ESI]: Calculated for C₂₆H₃₇N₇O₈Si [M+H⁺]: 604.25, Found: 604.3.

(E)-5-amino-6-((4-((2-amino-6-(3-((tert-butyldimethylsilyl)oxy)propoxy)-4-carbamoylphenyl)amino)but-2-en-1-yl)amino)nicotinamide (I-4-6): A stirred solution of compound I-4-5 (19 g, 0.031 mol) and aqueous ammonia (25% v/v, 66.5 mL, 3.9 mol) in methanol (760 mL) was cooled to 0° C. and then a solution of Na₂S₂O₄ (54 g, 0.314 mol) in water (156 mL) was added slowly, The reaction mixture was allowed to warm to room temperature and stirred for 20 minutes. Then the mixture was filtered and the filtrate was concentrated under reduce pressure at 20° C. The crude compound was purified by silica gel column chromatography (DCM:MeOH=65:35) to give compound I-4-6 (4.2 g) as a grey color solid. LC-MS [ESI]: Calculated for C₂₆H₄₁N₇O₄Si [M+H⁺]: 544.3, Found: 544.35.

Intermediate I-4: To a stirred solution of compound I-4-6 (200 mg, 0.36 mmol) in anhydrous DMF (10 mL) at 0° C. was added 1-ethyl-3-methyl-1H-pyrazole-5-carbonyl isothiocyanate (I-4-7, 2 eq) and the reaction mixture was stirred for 1 hour at room temperature and then slowly warmed to 40° C., after which DCC (227 mg, 1.1 mmol) was added and the mixture stirred for 16 hours at 40° C. The reaction mixture was purified by reverse phase preparative HPLC to give compound OTBS-protected Intermediate I-4 (100 mg). To a solution of OTBS protected Intermediate I-4 (100 mg) in MeOH (3 mL) was added a solution of HCl in 1,4 dioxane (4M, 1.5 mL) at 0° C. The reaction was stirred for 4 h at rt. The reaction was concentrated under reduced pressure. The pH of the residue was adjusted to 7-8 by adding LiOH solution (1M), and then the solution was concentrated. The residue was purification by purified by reverse phase preparative HPLC to give Intermediate I-4 (60 mg) as solid. LC-MS [ESI]: Calculated for C₃₆H₄₁N₁₃O₆ [M+H⁺]: 752.33, Found: 752.4

### Example 1B. Synthesis of Select STING Agonists

### Synthesis 1B-1. Compound S-1

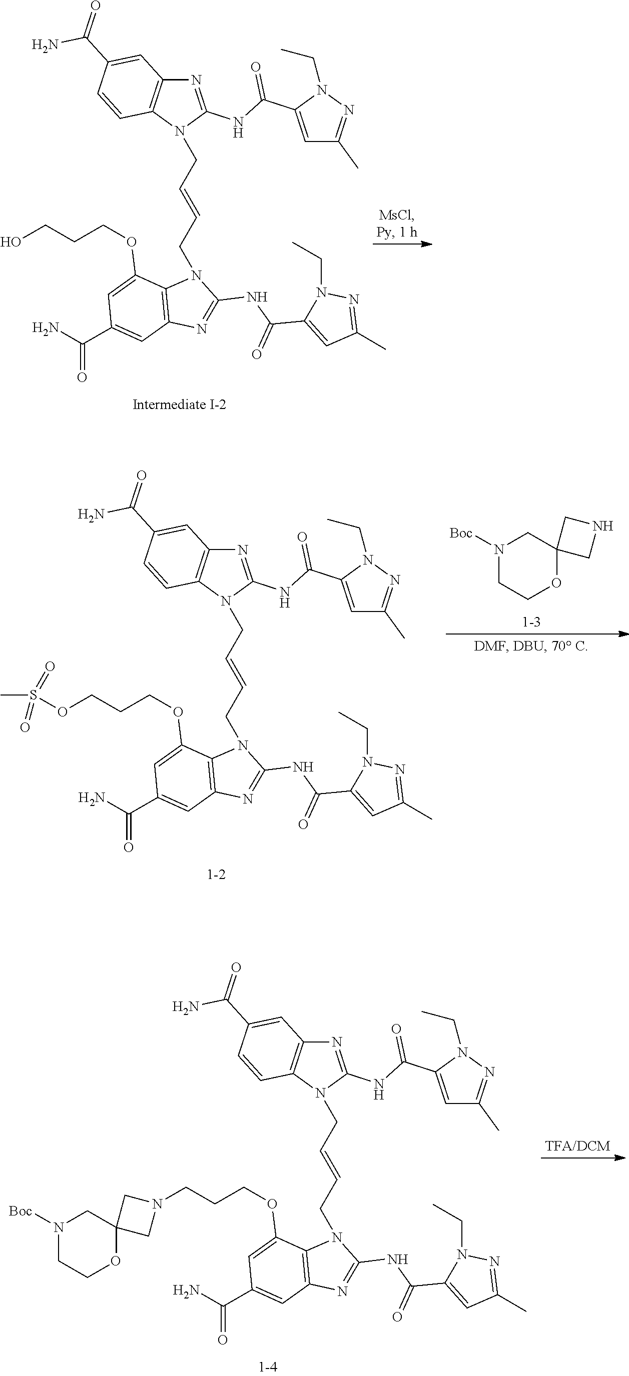

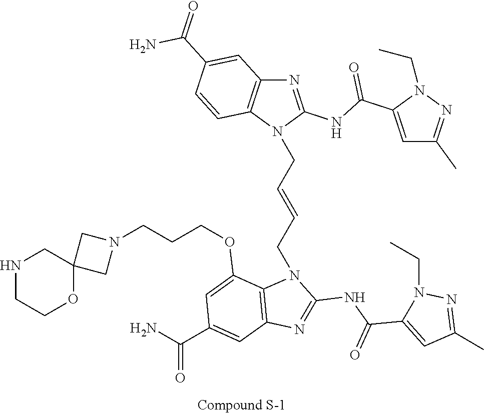 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21]

Compound 1-2: To a cooled solution (ice bath) of Intermediate I-2 (800 mg, 1.07 mmol) in anhydrous pyridine (10 mL) was added methanesulfonyl chloride (0.66 mL, 8.6 mmol) dropwise. The mixture was stirred at room temperature for a period of 1 h. Then the mixture was concentrated under reduced pressure and the crude material was purified by reverse phase Preparative HPLC to give compound 1-2 as a white solid after lyophilization (502 mg); LC-MS [ESI]: Calculated for C₃₈H₄₄N₁₂O₈S [M+H⁺]: 829.31, Found: 829.4.

Compound 1-4: The compound 1-2 (500 mg, 0.6 mmol)) was dissolved in anhydrous DMF (4 mL). To this solution, the diamine 1-3 (275 mg, 1.2 mmol) was added, followed by DBU (0.9 mmol, 0.13 mL), and the mixture was stirred at 70° C. for 16 h. The crude mixture was purified directly by reverse phase Preparative HPLC to give compound 1-4 as a white powder after lyophilization (280 mg); LC-MS [ESI]: Calculated for C₄₈H₆₀N₁₄O₈[M+H+]: 961.47, Found: 961.5.

Compound S-1: The compound 1-4 (60 mg, 62.5 mmol) was treated with TFA/DCM (1/4, v/v, 2 mL) at room temperature for 10 minutes. The reaction mixture was evaporated to dryness under reduced pressure and the residue was purified by reverse phase Preparative HPLC to give Compound S-1 as a white powder (44 mg, TFA salt); LC-MS [ESI]: Calculated for C₄₃H₅₂N₁₄O₆ [M+H⁺]: 861.42, Found: 861.55; ¹H NMR (400 MHz, DMSO) δ 12.83 (s, 1H), 10.39 (d, J=145.9 Hz, 1H), 9.18 (s, 1H), 7.97 (dd, J=9.4, 3.6 Hz, 1H), 7.87-7.53 (m, 1H), 7.36 (dd, J=14.0, 9.3 Hz, 2H), 6.53 (d, J=14.8 Hz, 1H), 5.97 (d, J=15.5 Hz, 1H), 5.69 (d, J=15.6 Hz, 1H), 4.88 (dd, J=53.5, 5.4 Hz, 2H), 4.67-4.27 (m, 3H), 4.27-3.54 (m, 8H), 3.12 (s, 4H), 2.13 (d, J=3.6 Hz, 2H), 1.85 (d, J=28.5 Hz, 1H), 1.27 (t, J=7.1 Hz, 2H).

### Synthesis 1B-2. Compound S-2

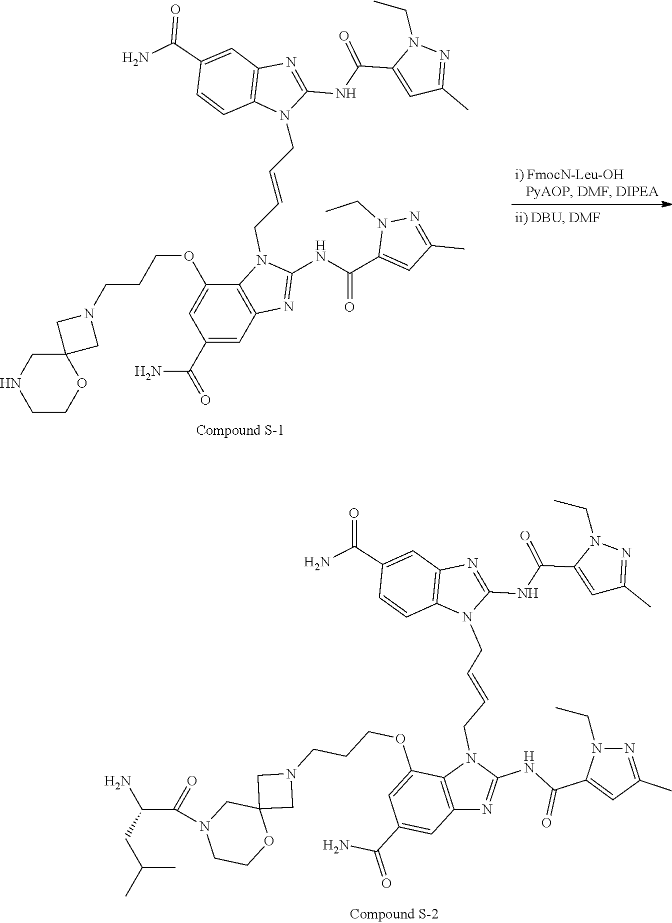 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21]

To a solution of Compound S-1 (TFA salt, 20 mg) and Fmoc-Leu-OH (7 mg, 20 mmol) in DMF (1 mL) was added PyAOP (10.4 mg, 20 mmol), followed by DIPEA (20 mL). After 10 min, DBU (40 mL) was added, and the mixture was stirred at room temperature for 20 min. The crude reaction was purified directly by reverse phase preparative HPLC to give Compound S-2 (14 mg, TFA salt) as a white powder. LC-MS [ESI]: Calculated for C₄₉H₆₃N₁₅O₇ [M+H⁺]: 974.5, Found: 974.7; ¹H NMR (400 MHz, DMSO) δ 12.83 (s, 2H), 8.18 (s, 1H), 8.13 (s, 2H), 7.97 (s, 2H), 7.73-7.64 (m, 2H), 7.39 (t, J=7.0 Hz, 3H), 7.32 (s, 1H), 6.53 (d, J=12.0 Hz, 2H), 5.71 (s, 1H), 4.94 (s, 2H), 4.81 (d, J=5.6 Hz, 2H), 4.53 (d, J=7.2 Hz, 4H), 4.36 (s, 1H), 4.07 (s, 4H), 3.93 (d, J=13.5 Hz, 1H), 3.46 (s, 1H), 3.40 (s, 1H), 2.12 (s, 6H), 1.83 (s, 2H), 1.70 (s, 1H), 1.56 (s, 1H), 1.49-1.39 (m, 1H), 1.27 (m, 6H), 0.95-0.84 (m, 6H).

### Synthesis 1B-3. Compound S-3

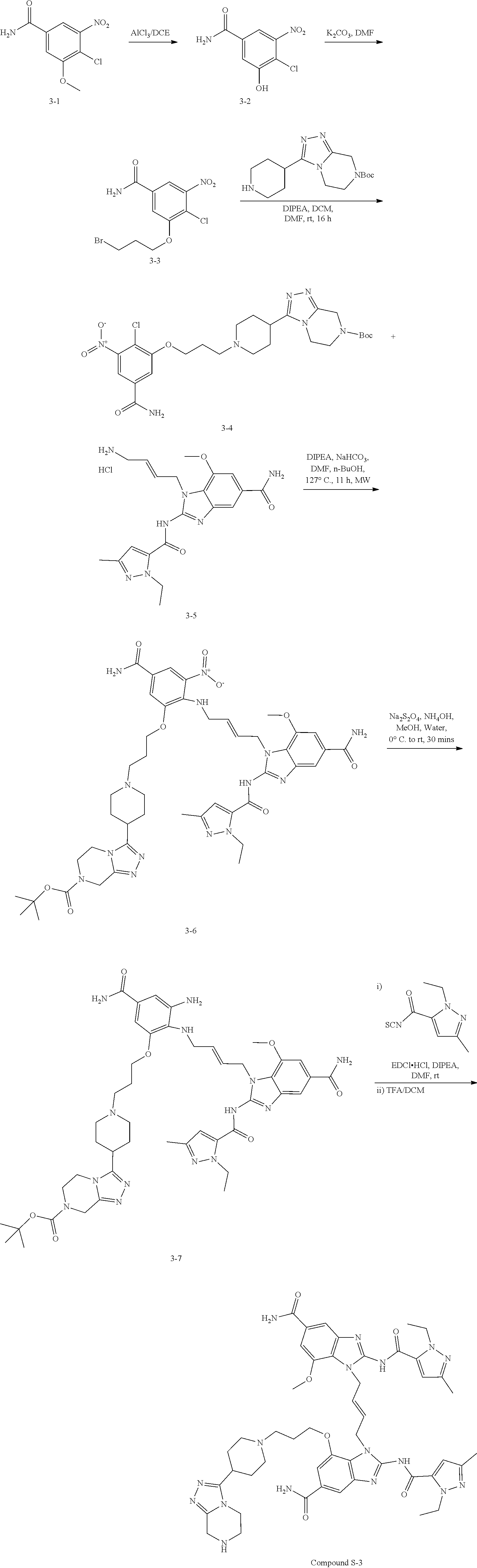

4-Chloro-3-hydroxy-5-nitro-benzamide (3-2): In a round bottomed flask, was taken 4-chloro-3-methoxy-5-nitro-benzamide (3-1) (25 g, 108.41 mmol). To this was added DCE (250 mL). To the resulting suspension at room temperature was added AlCl₃ (43.4 g, 325.22 mmol) portion wise. The suspension turned clear and the color changed to dark brown. The reaction mixture was stirred at rt for 16 h. Crushed ice was added to the reaction mixture followed by the addition of water. The reaction mixture was allowed to stir at rt for 2 h and the brown gum turned into an off-white suspension. Solids were filtered, washed with water followed by hexanes, and dried under reduced pressure to afford 4-chloro-3-hydroxy-5-nitro-benzamide (3-2) (21 g) as a light brown solid. ¹H NMR (400 MHz, DMSO) δ 11.57 (s, 1H), 8.19 (s, 1H), 7.92 (d, J=2.0 Hz, 1H), 7.73 (d, J=2.0 Hz, 1H), 7.67 (s, 1H). LC-MS [ESI]: Calculated for C₇H₅ClN₂O₄[M+H⁺]: 217.0, Found: 217.0.

3-(3-Bromopropoxy)-4-chloro-5-nitro-benzamide (3-3): To a stirred solution of 4-chloro-3-hydroxy-5-nitro-benzamide (3-2, 1.5 g, 6.93 mmol) in DMF (10 mL) at rt was added K₂CO₃ (23.9 g, 17.31 mmol) followed by the addition of 1,3-dibromopropane (1.41 mL, 13.85 mmol). The suspension was heated to 60° C. and stirred for 2 h. Reaction mixture was cooled to rt, cold water was added to the reaction mixture, solids were filtered, washed with water followed by hexanes, and dried under reduced pressure to afford 3-(3-bromopropoxy)-4-chloro-5-nitro-benzamide (3-3, 2.2 g) as an off-white solid. Crude compound (3-3) was used in the next step without further purification. LC-MS [ESI]: Calculated for C₁₀H₁₀BrClN₂O₄[M+H⁺]: 337.0, Found: 337.0.

tert-Butyl 3-[1-[3-(5-carbamoyl-2-chloro-3-nitro-phenoxy)propyl]-4-piperidyl]-6,8-dihydro-5H-[1,2,4]triazolo[4,3-a]pyrazine-7-carboxylate (3-4): To a stirred solution of tert-butyl 3-(4-piperidyl)-6,8-dihydro-5H-[1,2,4]triazolo[4,3-a]pyrazine-7-carboxylate hydrochloride 3-3 (2.1 g, 6.11 mmol) in DCM (25 mL) at rt was added DIPEA (2.34 mL, 13.44 mmol) followed by the addition of 3-(3-bromopropoxy)-4-chloro-5-nitro-benzamide (2.27 g, 6.72 mmol). The suspension was stirred at rt for 30 mins. To this was added DMF (15 mL) and the suspension was stirred at rt for 2 days. The reaction was diluted with water and extracted with DCM (3×100 mL). Emulsion was observed, and the biphasic layer was passed through a plug of celite. The two layers were separated and the combined organic part was dried over sodium sulfate before concentrating to dryness. The crude was then triturated with MTBE to afford tert-butyl 3-[1-[3-(5-carbamoyl-2-chloro-3-nitro-phenoxy)propyl]-4-piperidyl]-6,8-dihydro-5H-[1,2,4]triazolo[4,3-a]pyrazine-7-carboxylate (3-4, 1.3 g) as off white solid. ¹H NMR (400 MHz, DMSO) δ 8.31 (s, 1H), 8.05 (d, J=1.7 Hz, 1H), 7.89 (d, J=1.9 Hz, 1H), 7.79 (s, 1H), 4.63 (s, 2H), 4.29 (t, J=6.2 Hz, 2H), 3.96 (t, J=5.5 Hz, 2H), 3.74 (t, J=5.4 Hz, 2H), 2.94 (d, J=32.3 Hz, 2H), 2.73 (d, J=6.7 Hz, 1H), 2.10-1.61 (m, 8H), 1.44 (s, 9H). LC-MS [ESI]: Calculated for C₂₅H₃₄ClN₇O₆[M+H⁺]: 564.23, Found: 564.20.

tert-Butyl 3-[1-[3-[5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]-3-nitro-phenoxy]propyl]-4-piperidyl]-6,8-dihydro-5H-[1,2,4]triazolo[4,3-a]pyrazine-7-carboxylate (3-6): A stirred suspension of tert-butyl 3-[1-[3-(5-carbamoyl-2-chloro-3-nitro-phenoxy)propyl]-4-piperidyl]-6,8-dihydro-5H-[1,2,4]triazolo[4,3-a]pyrazine-7-carboxylate (3-4, 631.6 mg, 1.12 mmol), 1-[(E)-4-aminobut-2-enyl]-2-[(2-ethyl-5-methyl-pyrazole-3-carbonyl)amino]-7-methoxy-benzimidazole-5-carboxamide hydrochloride (3-4, 501.6 mg, 1.12 mmol), sodium bicarbonate (188 mg, 2.24 mmol) and DIPEA (0.78 mL, 4.48 mmol) in 1-butanol (10 mL) was heated to 127° C. under MW irradiation (at normal absorption level) for 11 h. LC-MS showed major desired product formation. Residue was suspended in DMF and filtered. Filtrate was purified by reverse phase preparative HPLC (Column: Phenomenex kinetex 5 μm C₁₈ 100 Å, 250×50 mm and using 5-40% ACN: 1% TFA in water as eluent, Flow rate: 118.1 mL/min, run time: 25 mins to afford tert-butyl 3-[1-[3-[5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]-3-nitro-phenoxy]propyl]-4-piperidyl]-6,8-dihydro-5H-[1,2,4]triazolo[4,3-a]pyrazine-7-carboxylate (3-6, 320 mg, 0.34 mmol) as white solid. ¹H NMR (400 MHz, DMSO) δ 9.32 (s, 1H), 8.16 (dd, J=13.6, 1.8 Hz, 1H), 8.01 (s, 1H), 7.66 (dd, J=4.8, 1.3 Hz, 1H), 7.50 (d, J=2.0 Hz, 1H), 7.44-7.29 (m, 3H), 6.60 (d, J=3.7 Hz, 1H), 5.79 (dt, J=15.7, 5.6 Hz, 1H), 5.67 (dt, J=15.6, 5.4 Hz, 1H), 4.90 (d, J=5.6 Hz, 3H), 4.69 (s, 4H), 4.57 (q, J=7.1 Hz, 3H), 4.14 (d, J=5.8 Hz, 3H), 3.99 (ddt, J=18.1, 12.4, 6.3 Hz, 5H), 3.86 (d, J=2.2 Hz, 3H), 3.79 (t, J=5.5 Hz, 2H), 3.55 (d, J=11.8 Hz, 2H), 3.23-3.09 (m, 2H), 3.00 (dt, J=21.7, 7.8 Hz, 3H), 2.16 (s, 4H), 2.12-2.03 (m, 3H), 2.02-1.88 (m, 2H), 1.45 (d, J=2.4 Hz, 9H), 1.32 (td, J=7.1, 2.0 Hz, 3H). LC-MS [ESI]: Calculated for C₄₅H₅₈N₁₄O₉[M+H⁺]: 939.46, Found: 939.50.

tert-Butyl 3-[1-[3-[3-amino-5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]phenoxy]propyl]-4-piperidyl]-6,8-dihydro-5H-[1,2,4]triazolo[4,3-a]pyrazine-7-carboxylate (3-7): To a stirred suspension of tert-butyl 3-[1-[3-[5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]-3-nitro-phenoxy]propyl]-4-piperidyl]-6,8-dihydro-5H-[1,2,4]triazolo[4,3-a]pyrazine-7-carboxylate (3-6) (100 mg, 0.11 mmol) in methanol (3 mL) at 0° C. was added sodium hydrosulfite (185.4 mg, 1.06 mmol) in water (1 mL) followed by the simultaneous addition of ammonium hydroxide (0.69 mL, 5.32 mmol). The resulting suspension was allowed to warm to room temperature and stirred for 2 h. LCMS showed the reaction was completed. Reaction mixture was concentrated under reduced pressure before it was suspended in DMF and filtered through a plug. Filtrate was purified by reverse phase preparative HPLC (Column: Phenomenex kinetex 5 μm C₁₈ 100 Å, 250×21.2 mm and using 5-40% ACN: 0.1% TFA in water as eluent, Flow rate: 21.2 mL/min, run time: 25 mins to afford tert-butyl 3-[1-[3-[3-amino-5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]phenoxy]propyl]-4-piperidyl]-6,8-dihydro-5H-[1,2,4]triazolo[4,3-a]pyrazine-7-carboxylate (3-7) (59 mg, 0.06 mmol) as white solid. ¹H NMR (400 MHz, MeOD) δ 7.67 (d, J=1.4 Hz, 1H), 7.39 (dd, J=3.8, 1.6 Hz, 1H), 6.94 (d, J=1.7 Hz, 1H), 6.69 (s, 1H), 6.65 (d, J=1.8 Hz, 1H), 6.04 (dt, J=15.4, 5.1 Hz, 1H), 5.48 (dt, J=14.6, 6.8 Hz, 1H), 5.02-4.96 (m, 2H), 4.66 (q, J=7.1 Hz, 2H), 4.12 (s, 2H), 4.02-3.97 (m, 2H), 3.97-3.93 (m, 2H), 3.92 (s, 2H), 3.89-3.79 (m, 2H), 3.23 (d, J=16.3 Hz, 2H), 2.27 (d, J=5.6 Hz, 5H), 2.09-1.99 (m, 2H), 1.53 (d, J=3.1 Hz, 9H), 1.41 (t, J=7.1 Hz, 3H). LC-MS [ESI]: Calculated for C₄₅H₆₀N₁₄O₇[M+H⁺]: 909.40, Found: 909.40.

tert-Butyl 3-[1-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate: To a stirred solution of tert-butyl 3-[1-[3-[3-amino-5-carbamoyl-2-[[(E)-4-[5-carbamoyl-2-[(2-ethyl-5-methyl-pyrazole-3-carbonyl)amino]-7-methoxy-benzimidazol-1-yl]but-2-enyl]amino]phenoxy]propyl]-4-piperidyl]-6,8-dihydro-5H-[1,2,4]triazolo[4,3-a]pyrazine-7-carboxylate (3-7) (25 mg, 0.03 mmol) in DMF (1 mL) at rt was added DIPEA (0.02 mL, 0.14 mmol) followed by the addition of 2-ethyl-5-methyl-pyrazole-3-carbonyl isothiocyanate (5.91 mg, 0.03 mmol). The resulting yellow solution was stirred at rt for 2 h followed by at 40° C. for 1 h. The reaction mixture was cooled to rt and to this was added N,N′-dicyclohexylmethanediimine (5.6 mg, 0.03 mmol). The reaction mixture was warmed to 40° C. and stirred at this temperature for 16 h. Reaction mixture was cooled to rt and purified by reverse phase preparative HPLC (Column: Phenomenex kinetex 5 μm C₁₈ 100 Å, 250×21.2 mm and using 5-70% ACN: 0.1% TFA in water as eluent, Flow rate: 21.2 mL/min, run time: 30 mins to afford tert-butyl 3-[1-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (7 mg) as white solid. LC-MS [ESI]: Calculated for C₅₃H₆₇N₁₇O₈[M+H⁺]: 1070.54, Found: 1070.60.

1-[(E)-4-[5-Carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2-ethyl-5-methyl-pyrazole-3-carbonyl)amino]-7-methoxy-benzimidazole-5-carboxamide (Compound S-3): To a stirred solution of tert-butyl 3-[1-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]-4-piperidyl]-6,8-dihydro-5H-[1,2,4]triazolo[4,3-a]pyrazine-7-carboxylate (7 mg, 0.01 mmol) in HFIP (0.5 mL) at rt was added 5% TFA in HFIP (1. mL, 0.65 mmol). The solution was stirred at rt for 2 h. Reaction mixture was concentrated under reduced pressure before it was taken in DMF and purified by reverse phase preparative HPLC (Column: Phenomenex kinetex 5 μm C₁₈ 100 Å, 250×21.2 mm and using 5-35% ACN: 0.1% TFA in water as eluent, Flow rate: 21.2 mL/min, run time: 25 mins to afford 1-[(E)-4-[5-carbamoyl-2-[(2-ethyl-5-methyl-pyrazole-3-carbonyl)amino]-7-[3-[4-(5,6,7,8-tetrahydro-[1,2,4]triazolo[4,3-a]pyrazin-3-yl)-1-piperidyl]propoxy]benzimidazol-1-yl]but-2-enyl]-2-[(2-ethyl-5-methyl-pyrazole-3-carbonyl)amino]-7-methoxy-benzimidazole-5-carboxamide (Compound S-3, 3 mg) as white solid (di TFA salt). ¹H NMR (400 MHz, DMSO) δ 12.89 (s, 2H), 9.38 (s, 2H), 8.00 (d, J=20.8 Hz, 2H), 7.70-7.62 (m, 2H), 7.40 (s, 2H), 7.33 (d, J=11.0 Hz, 2H), 6.54 (d, J=4.6 Hz, 2H), 5.90-5.70 (m, 3H), 4.92 (d, J=15.3 Hz, 4H), 4.53 (d, J=13.8 Hz, 6H), 4.29-3.94 (m, 6H), 3.69 (d, J=30.5 Hz, 7H), 3.18-2.84 (m, 7H), 2.12 (d, J=10.5 Hz, 7H), 1.94 (d, J=13.0 Hz, 5H), 1.29 (dt, J=11.2, 7.1 Hz, 6H). LC-MS [ESI]: Calculated for C₄₈H₅₉N₁₇O₆[M+H⁺]: 970.50, Found: 970.50.

### Synthesis 1B-4. Compound S-4 and Compound S-5

Compound S-4 and Compound S-5 were synthesized as shown below via general Scheme 6.

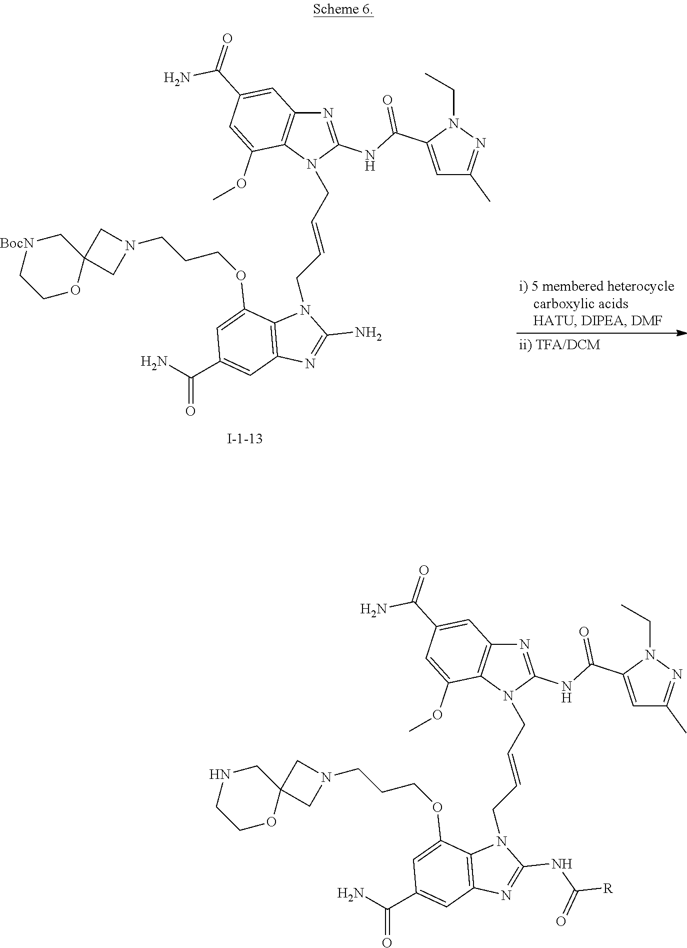

Step 1: Amide coupling of compound I-1-13 with corresponding heterocycle carboxylic acids: To a mixture of tert-butyl (E)-2-(3-((2-amino-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-7-yl)oxy)propyl)-5-oxa-2,8-diazaspiro[3.5]nonane-8-carboxylate (I-1-13) (1 eq) and the corresponding five membered heterocycle carboxylic acid in DMF, was added DIPEA (3 eq) and HATU (1.2 eq). The mixture was stirred at 50° C. and a clear solution formed. The reaction was stirred for 5 hours to drive the reaction to completion. The reaction solution was purified by reverse phase preparative HPLC (acetonitrile/water+0.1% formic acid) to afford Boc-protected Compound S-4 and Compound S-5.

Step 2: Boc removal: A mixture of the Boc-protected intermediate (16.4 mg) in 2 mL of 5% (v/v) trifluoracetic acid in 1,1,1,3,3,3-hexafluoroisopropanol (HFIP) was stirred at room temperature for 0.5 hour. The reaction mixture was concentrated. The residue was diluted with DMSO (2 mL) and purified by reverse phase preparative HPLC (acetonitrile/water+0.1% TFA) to afford the final compound as its TFA salt. The final compounds were characterized by LCMS, ¹HNMR.

S-4 NMR and LC-MS data: ¹H NMR (400 MHz, DMSO) δ 8.99 (s, 2H), 7.97 (s, 2H), 7.64 (s, 2H), 7.38 (s, 1H), 7.31 (s, 2H), 6.52 (s, 2H), 5.79 (s, 2H), 4.91 (d, J=14.4 Hz, 5H), 4.53 (s, 4H), 4.20-3.67 (m, 13H), 3.44 (s, 6H), 3.12 (s, 9H), 2.12 (d, J=1.6 Hz, 6H), 1.78 (s, 3H), 1.27 (t, J=7.1 Hz, 6H). LC-MS [ESI]: Calculated for C₄₄H₅₅N₁₄O₇ [M+H⁺]: 891.43, Found: 891.5.

S-5 NMR and LC-MS data: 1H NMR (400 MHz, DMSO) δ 7.96 (s, 2H), 7.67-7.61 (m, 2H), 7.38-7.25 (m, 4H), 6.51 (d, J=15.3 Hz, 2H), 5.81 (dt, J=10.9, 5.1 Hz, 2H), 4.90 (s, 4H), 4.51 (q, J=7.1 Hz, 2H), 3.95 (t, J=6.0 Hz, 2H), 3.73 (s, 3H), 3.42 (s, 2H), 3.23 (d, J=7.1 Hz, 2H), 2.85 (dd, J=15.7, 8.2 Hz, 4H), 2.66 (s, 3H), 2.40 (s, 3H), 2.10 (s, 3H), 1.91 (d, J=0.8 Hz, 3H), 1.59-1.51 (m, 2H), 1.26 (q, J=6.7 Hz, 3H), 1.05 (t, J=7.5 Hz, 3H). LC-MS [ESI]: Calculated for C₄₄H₅₄N₁₃O₈[M+H⁺]: 892.41, Found: 892.5

| Compound No. | Structure |
|---|---|
| S-4 | 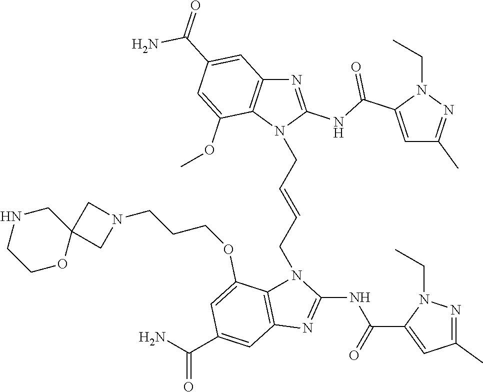 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(CNCCO4)C3)c21] |
| S-5 | 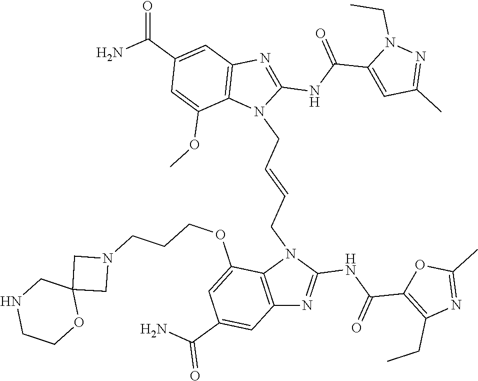 [SMILES: CCc1nc(C)oc1C(=O)Nc1nc2cc(C(N)=O)cc(OCCCN3CC4(CNCCO4)C3)c2n1C/C=C/Cn1c(NC(=O)c2cc(C)nn2CC)nc2cc(C(N)=O)cc(OC)c21] |

### Synthesis 1B-5. Compound S-6

Compound S-6 was synthesized as shown below via general Scheme 7 (via Compound S-4) by coupling Compound S-4 to Boc-protected L-leucine in the presence of HBTU and DIPEA in DMF. Boc-protected Compound S-6 was deprotected using TFA to afford Compound S-6. The final compound was purified by reverse phase preparative HPLC and characterized by LCMS and ¹HNMR. ¹H NMR (400 MHz, DMSO) δ 7.94 (s, 2H), 7.65 (d, J=5.1 Hz, 2H), 7.45-7.20 (m, 3H), 6.54 (d, J=19.3 Hz, 2H), 5.83 (dt, J=8.8, 4.9 Hz, 2H), 4.92 (d, J=16.7 Hz, 3H), 4.54 (p, J=7.0 Hz, 3H), 3.96 (s, 2H), 3.72 (s, 3H), 3.48 (d, J=21.9 Hz, 4H), 3.11 (s, 1H), 2.62 (d, J=7.3 Hz, 1H), 2.41 (s, 1H), 2.12 (d, J=7.8 Hz, 4H), 1.83-1.66 (m, 1H), 1.54 (s, 2H), 1.35-1.14 (m, 6H), 0.94-0.70 (m, 4H). LC-MS [ESI]: Calculated for C₅₀H₆₆N₁₅O₈ [M+H⁺]: 1004.51, Found: 1004.52

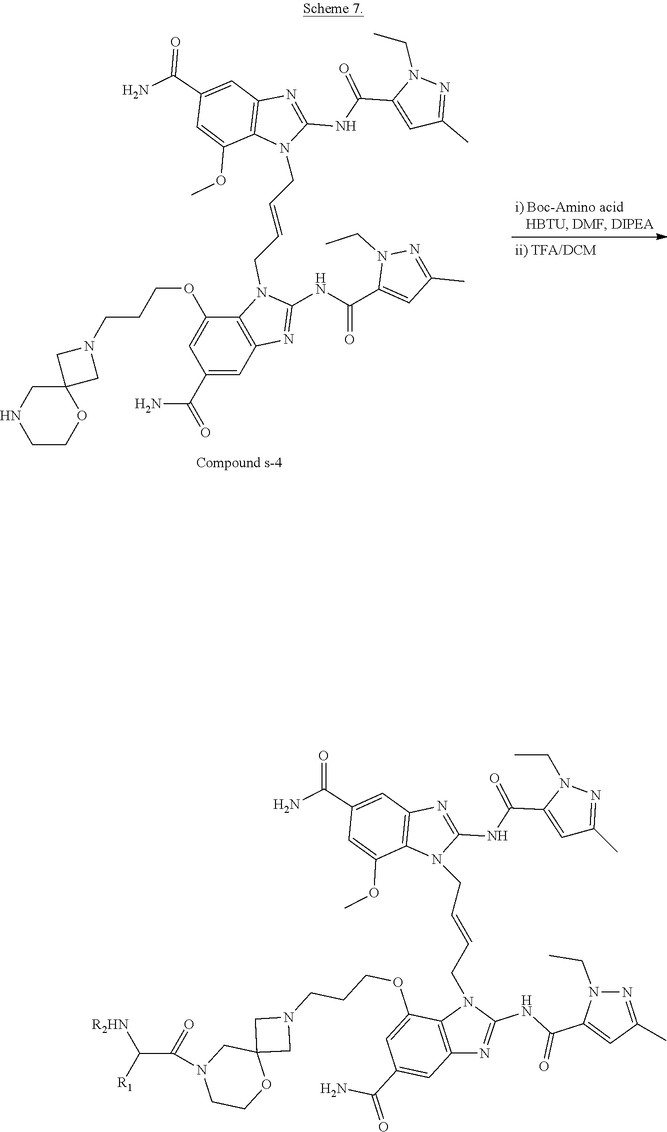

| Compound No. | Structure |
|---|---|
| S-6 | 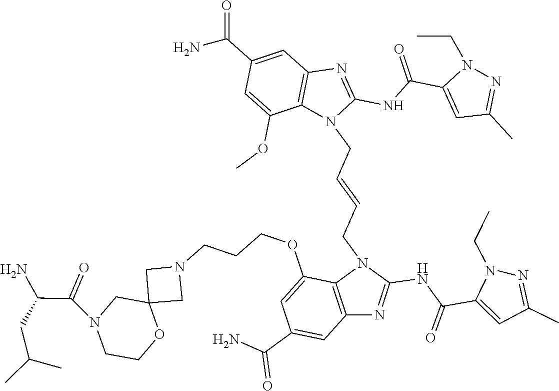 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)[C@@H](N)CC(C)C)CCO4)c21] |

### Synthesis 1B-6. Compound S-7

Compound S-7 was synthesized using the same methods as described herein for the synthesis of Compound S-1, but starting from Intermediate I-4.

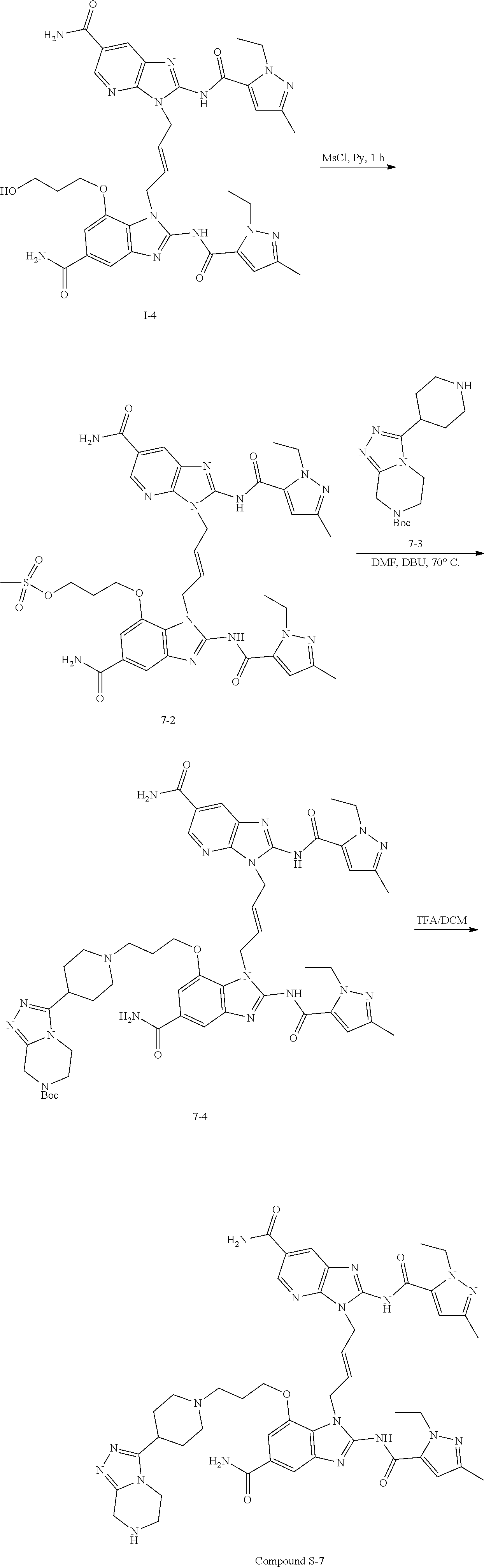

### Synthesis 1B-7. Compound S-9

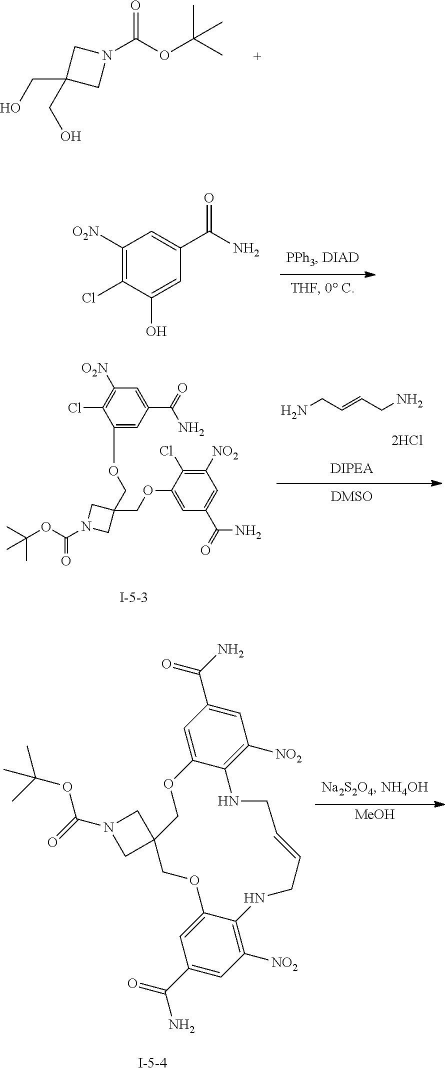

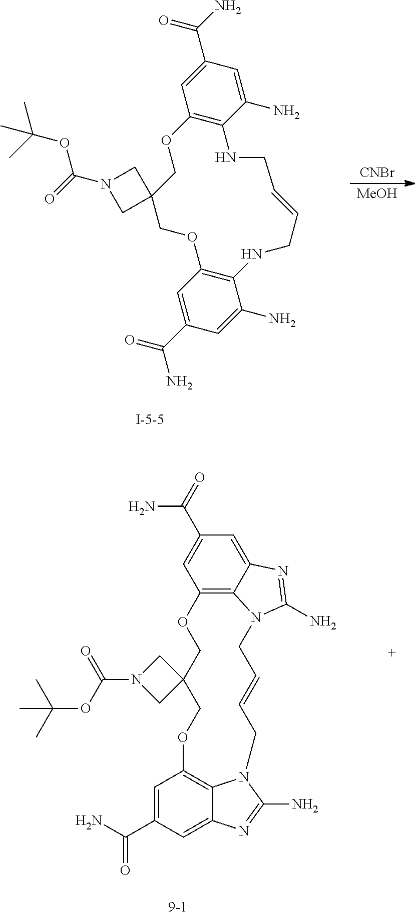 [SMILES: "MeOH" ; CC(C)(C)OC(=O)N1CC2(COc3cc(C(N)=O)cc(N)c3NC/C=C/CNc3c(N)cc(C(N)=O)cc3OC2)C1 ; "CNBr" ; CC(C)(C)OC(=O)N1CC2(COc3cc(C(N)=O)cc4nc(N)n(c34)C/C=C/Cn3c(N)nc4cc(C(N)=O)cc(c43)OC2)C1]

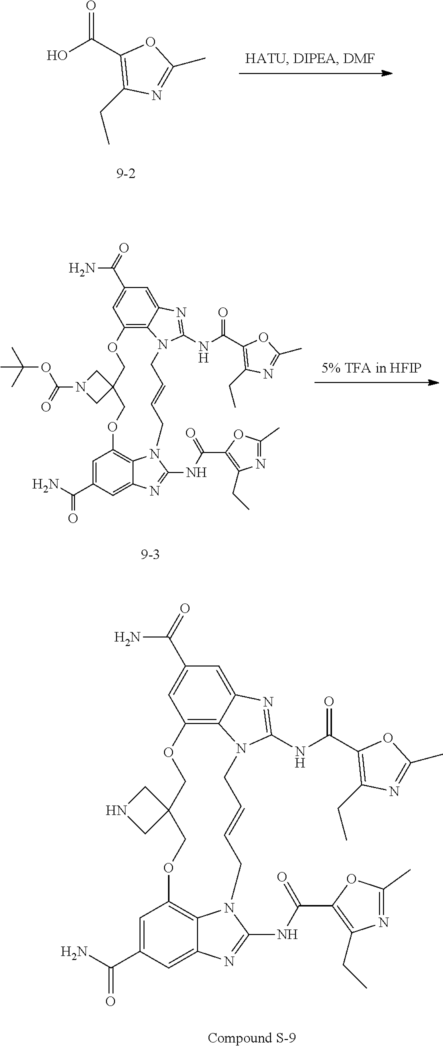 [SMILES: CCc1nc(C)oc1C(=O)O ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OC(C)(C)C)C1 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CNC1)CO3]

tert-Butyl 3,3-bis[(5-carbamoyl-2-chloro-3-nitro-phenoxy)methyl]azetidine-1-carboxylate (I-5-3): To a stirred solution of tert-butyl 3,3-bis(hydroxymethyl)azetidine-1-carboxylate (500.0 mg, 2.3 mmol), 4-chloro-3-hydroxy-5-nitro-benzamide (996.9 mg, 4.6 mmol) and triphenylphosphine (1811 mg, 6.9 mmol) in THF (20 mL) at 0° C. was added dropwise isopropyl (NE)-N-isopropoxycarbonyliminocarbamate (DIAD, 1.36 mL, 6.9 mmol). The reaction mixture was allowed to warm to room temperature and stirred for 16 h. The reaction mixture was concentrated under reduced pressure. Crude material was purified by silica gel column chromatography (solid load, 24 g cartridge, 20-100% EA/hexanes as eluent). Pure fractions were combined and concentrated to give tert-butyl 3,3-bis[(5-carbamoyl-2-chloro-3-nitro-phenoxy)methyl]azetidine-1-carboxylate (I-5-3) (401 mg, 0.65 mmol, 28.36% yield) as white solid. ¹H NMR (400 MHz, DMSO) δ 8.28 (s, 2H), 8.10 (d, J=1.8 Hz, 2H), 7.97 (d, J=1.9 Hz, 2H), 7.81 (s, 2H), 4.53 (s, 4H), 3.94 (s, 4H), 1.40 (s, 9H). LC-MS [ESI]: Calculated for C₂₄H₂₆Cl₂N₄O₉ [M+H⁺]: 585.11, Found: 585.2.

tert-Butyl (15E)-9,22-dicarbamoyl-11,20-dinitro-spiro[2,6-dioxa-13,18-diazatricyclo[17.4.0.07,12]tricosa-1(23),7,9,11,15,19,21-heptaene-4,3′-azetidine]-1′-carboxylate (I-5-4): In a 20 mL vial, a suspension of tert-butyl 3,3-bis[(5-carbamoyl-2-chloro-3-nitro-phenoxy)methyl]azetidine-1-carboxylate (I-5-3) (460 mg, 0.75 mmol), DIPEA (0.63 mL, 4.49 mmol) and (E)-but-2-ene-1,4-diamine dihydrochloride (238.1 mg, 1.5 mmol) in DMSO (7.5 mL) was irradiated at normal absorption level at 120° C. for 3.5 h. The crude material was mixed with additional previously obtained crude material purified by prep-HPLC (Column: Phenomenex kinetex 5 μm C₁₈ 100 Å, 250×50 mm and using 10-70% ACN:10 mM NH4OAc in water as eluent, Flow rate: 118 mL/min, run time: 30 mins to afford tert-butyl (15E)-9,22-dicarbamoyl-11,20-dinitro-spiro[2,6-dioxa-13,18-diazatricyclo[17.4.0.07,12]tricosa-1(23),7,9,11,15,19,21-heptaene-4,3′-azetidine]-1′-carboxylate (I-5-4) (200 mg, 0.32 mmol, 42.5% yield) as brown solid. ¹H NMR (400 MHz, DMSO) δ 8.30 (d, J=1.9 Hz, 2H), 8.03 (s, 2H), 7.82 (d, J=2.1 Hz, 2H), 7.78 (t, J=6.6 Hz, 2H), 7.39 (s, 2H), 5.68 (d, J=1.8 Hz, 2H), 4.25 (s, 4H), 4.07 (d, J=6.5 Hz, 4H), 3.91 (s, 4H), 1.41 (s, 9H). LC-MS [ESI]: Calculated for C₂₈H₃₃N₇O₁₀ [M+H⁺]: 628.23, Found: 628.20.

tert-Butyl (15E)-11,20-diamino-9,22-dicarbamoyl-spiro[2,6-dioxa-13,18-diazatricyclo[17.4.0.07,12]tricosa-1(19),7(12),8,10,15,20,22-heptaene-4,3′-azetidine]-1′-carboxylate (I-5-5): To a stirred suspension of tert-butyl (15E)-9,22-dicarbamoyl-11,20-dinitro-spiro[2,6-dioxa-13,18-diazatricyclo[17.4.0.07,12]tricosa-1(19),7(12),8,10,15,20,22-heptaene-4,3′-azetidine]-1′-carboxylate (I-5-4, 100 mg, 0.16 mmol) in methanol (5 mL) at 0° C. was added sodium hydrosulfite (277.4 mg, 1.59 mmol) in H₂O (1 mL) followed by the simultaneous addition of ammonium hydroxide (1 mL, 7.9 mmol). The resulting suspension was allowed to warm to room temperature and stirred at room temperature for 2 h. LCMS showed the reaction was complete. Reaction mixture was filtered and solids were washed with MeOH. Combined filtrate was concentrated and purified by reverse phase preparative HPLC (Column: Phenomenex kinetex 5 μm C₁₈ 100 Å, 250×21.2 mm and using 10-50% ACN: 10 mM NH4OAc in water as eluent, Flow rate: 21.2 mL/min, run time: 30 mins to give tert-butyl (15E)-11,20-diamino-9,22-dicarbamoyl-spiro[2,6-dioxa-13,18-diazatricyclo[17.4.0.07,12]tricosa-1(19),7(12),8,10,15,20,22-heptaene-4,3′-azetidine]-1′-carboxylate (I-5-5) (21 mg) as white solid. ¹H NMR (400 MHz, DMSO) δ 7.62 (s, 2H), 7.11-6.93 (m, 4H), 6.90 (d, J=1.8 Hz, 2H), 5.38 (t, J=3.0 Hz, 2H), 4.69 (s, 4H), 4.33 (s, 4H), 3.86 (d, J=28.7 Hz, 6H), 3.46 (s, 4H), 1.41 (s, 9H). LC-MS [ESI]: Calculated for C₂₈H₃₇N₇O₆ [M+H⁺]: 568.28, Found: 568.20.

tert-Butyl (3E)-7,25-diamino-11,21-dicarbamoyl-spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (Intermediate 9-1): To a stirred solution of tert-butyl (15E)-11,20-diamino-9,22-dicarbamoyl-spiro[2,6-dioxa-13,18-diazatricyclo[17.4.0.07,12]tricosa-1(19),7(12),8,10,15,20,22-heptaene-4,3′-azetidine]-1′-carboxylate (I-5-5, 20 mg, 0.04 mmol) in methanol (1 mL) at room temperature was added cyanogen bromide (30 mg, 0.28 mmol). The resulting solution was stirred at this temperature for 2 h. MeOH was concentrated under reduced pressure, residue was diluted with MTBE, suspension was filtered, and solids were washed with MTBE and dried under reduced pressure to afford tert-butyl (3E)-7,25-diamino-11,21-dicarbamoyl-spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (Intermediate 9-1) (17 mg) as light yellow solid. ¹H NMR (400 MHz, DMSO) δ 12.98 (s, 2H), 8.65 (s, 3H), 8.10 (s, 2H), 7.86 (s, 2H), 7.67-7.37 (m, 4H), 5.38 (s, 2H), 4.71 (s, 4H), 4.52 (s, 4H), 3.86 (s, 4H), 1.44 (s, 9H). LC-MS [ESI]: Calculated for C₃₀H₃₅N₉O₆ [M+H⁺]: 618.28, Found: 618.40.

tert-butyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (9-3): To a stirred solution of 4-ethyl-2-methyl-oxazole-5-carboxylic acid (9-2, 26.1 mg, 0.17 mmol) in DMF (1 mL) at room temperature was added HATU (74 mg, 0.19 mmol). The solution was stirred at room temperature for 15 minutes. In another vial, to a stirred solution of tert-butyl (3E)-7,25-diamino-11,21-dicarbamoyl-spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (9-1, 20 mg, 0.03 mmol) in DMF (1 mL) at room temperature (also referred as rt) was added DIPEA (0.05 mL, 0.32 mmol). To this at room temperature was added the above active ester solution and the reaction mixture was stirred at room temperature for 2 h. The reaction mixture was cooled to rt, cold water was added to the reaction mixture, solids were filtered, washed with water followed by hexanes, and dried under reduced pressure to afford the crude. The crude material was purified by reverse phase preparative HPLC (Column: Phenomenex kinetex 5 μm C₁₈ 100 Å, 250×21.2 mm and using 5-70% ACN: 1% TFA in water as eluent, Flow rate: 21.2 mL/min, run time: 30 mins to afford tert-butyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (9-3, 11 mg) as white solid. LC-MS [ESI]: Calculated for C₄₄H₄₉N₁₁O₁₀ [M+H⁺]: 892.40, Found: 892.40.

(3E)-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-11,21-dicarboxamide (Compound S-9): To a stirred solution of tert-butyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (9-3, 10 mg, 0.01 mmol) in HFIP (0.5 mL) at rt was added 5% TFA in HFIP (0.86 mL, 0.56 mmol). The solution was stirred at rt for 2 h. Reaction mixture was concentrated under reduced pressure and crude was purified by prep-HPLC (Column: Phenomenex kinetex 5 μm C₁₈ 100 Å, 250×21.2 mm and using 5-50% ACN: 1% TFA in water as eluent, Flow rate: 21.2 mL/min, run time: 30 mins to afford (3E)-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-11,21-dicarboxamide Compound S-9 (7 mg) as white solid (di TFA salt). ¹H NMR (400 MHz, DMSO) δ 12.85 (s, 2H), 9.02 (s, 2H), 7.99 (s, 2H), 7.79-7.75 (m, 2H), 7.73 (s, 2H), 7.46 (s, 2H), 5.38 (s, 2H), 4.83 (s, 4H), 4.55 (s, 4H), 4.08 (t, J=6.2 Hz, 4H), 2.79 (q, J=7.5 Hz, 4H), 2.37 (s, 6H), 0.98 (t, J=7.5 Hz, 6H). LC-MS [ESI]: Calculated for C₃₉H₄₁N₁₁O₈[M+H⁺]: 792.32, Found: 792.40.

### Synthesis 1B-8. Compound S-13

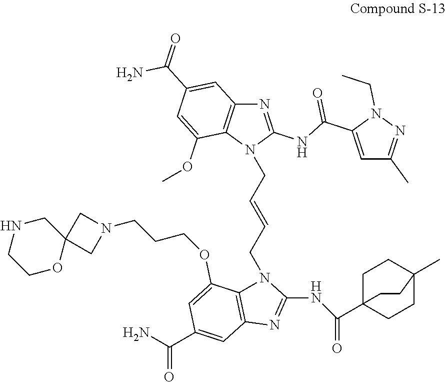 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(CNCCO4)C3)c21]

Compound S-13 was synthesized as shown in the general scheme below.

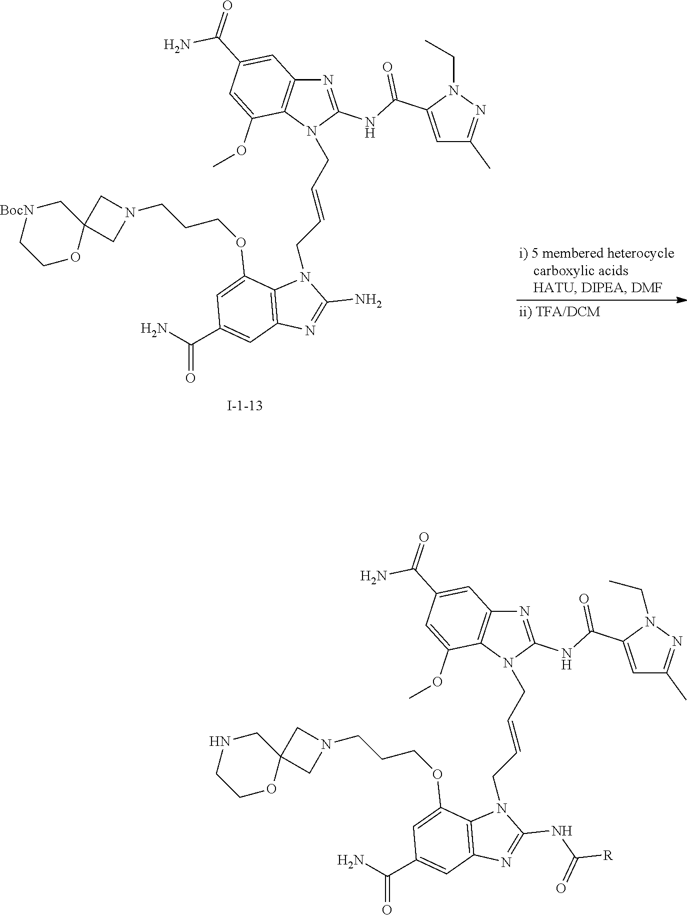

Step 1: Amide coupling of compound I-1-13 with corresponding heterocycle carboxylic acids: To a mixture of tert-butyl (E)-2-(3-((2-amino-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-7-yl)oxy)propyl)-5-oxa-2,8-diazaspiro[3.5]nonane-8-carboxylate (I-1-13) (1 eq) and the corresponding five membered heterocycle carboxylic acid in DMF, was added DIPEA (3 eq) and HATU (1.2 eq). The mixture was stirred at 50° C. and a clear solution formed. The reaction was stirred for 5 hours to drive the reaction to completion. The reaction solution was purified by reverse phase preparative HPLC (acetonitrile/water+0.1% formic acid) to afford Boc-protected Compound S-13.

Step 2: Boc removal: A mixture of the Boc-protected intermediate (16.4 mg) in 2 mL of 5% (v/v) trifluoracetic acid in 1,1,1,3,3,3-hexafluoroisopropanol (HFIP) was stirred at room temperature for 0.5 hour. The reaction mixture was concentrated. The residue was diluted with DMSO (2 mL) and purified by reverse phase preparative HPLC (acetonitrile/water+0.1% TFA) to afford the final compound as its TFA salt. S-13 was characterized by LCMS, ¹HNMR. ¹H NMR (400 MHz, DMSO) δ 7.96 (s, 2H), 7.67 (s, 1H), 7.34 (d, J=8.1 Hz, 2H), 7.25 (s, 2H), 6.54 (s, 1H), 5.80 (s, 2H), 4.86 (s, 2H), 4.75 (s, 1H), 4.52 (s, 2H), 3.99 (s, 2H), 3.73 (s, 3H), 3.38 (s, 1H), 3.22 (d, J=7.2 Hz, 2H), 2.73 (s, 2H), 2.63 (d, J=7.2 Hz, 2H), 2.59 (s, 4H), 2.13 (s, 3H), 1.91 (s, 2H), 1.61 (s, 9H), 1.24 (d, J=9.5 Hz, 10H), 0.74 (s, 3H). LC-MS [ESI]: Calculated for C₄₇H₆₁N₁₂O₇ [M+H⁺]: 905.47, Found: 905.5

### Example 1C. Synthesis of Select STING Agonist Linker-Payloads

### Synthesis 1C-1. LP-S1-1

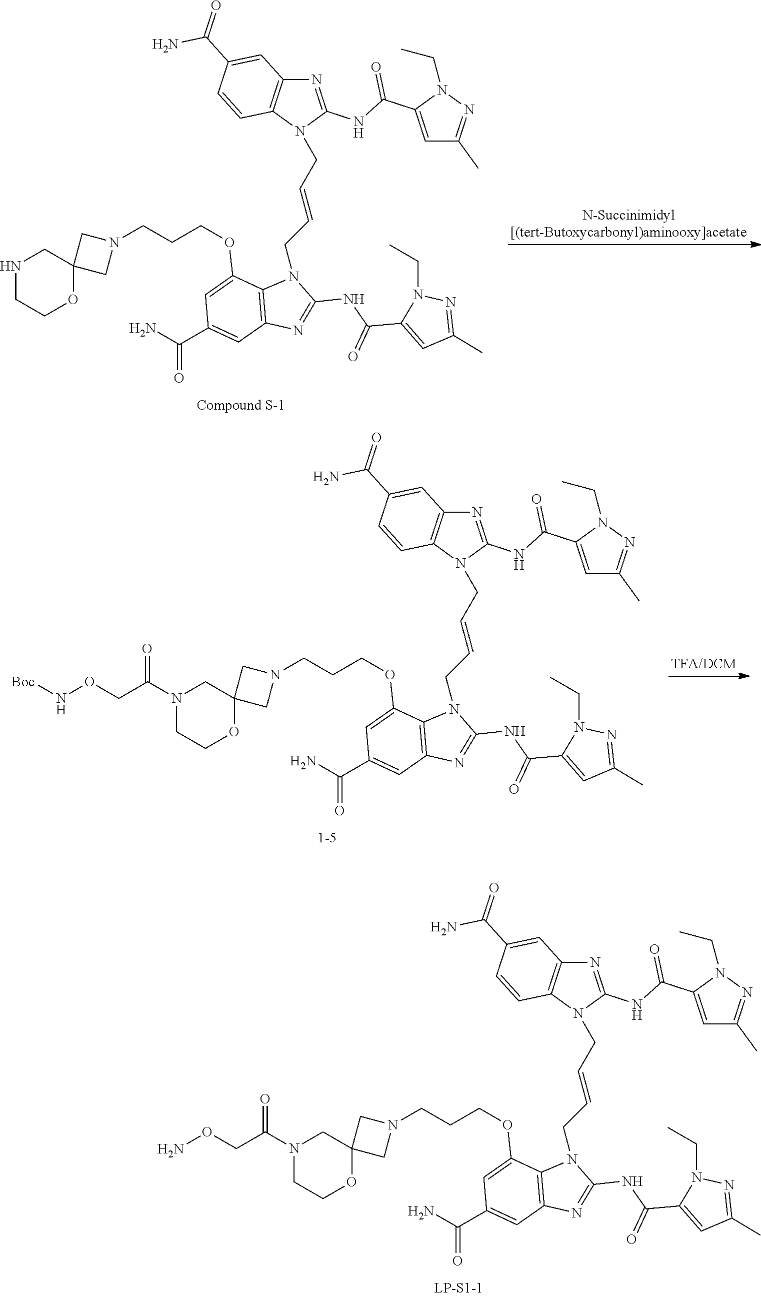 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CONC(=O)OC(C)(C)C)CCO4)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)CON)CCO4)c21]

Compound 1-5: Compound S-1 (80 mg) was dissolved in anhydrous DMF (4 mL). To this solution, N-succinimidyl[(tert-butoxycarbonyl)aminooxy]acetate (TCI, 29 mg, 0.1 mmol) was added, followed by DIPEA (0.4 mmol, 0.07 mL), and the mixture was stirred at room temperature for 1 h. The crude mixture was purified directly by reverse phase preparative HPLC to give compound 1-5 as a white powder after lyophilization (71 mg); LC-MS [ESI]: Calculated for C₅₀H₆₃N₁₅O₁₀ [M+H⁺]: 1034.49, Found: 1034.6.

LP-S1-1: Compound 1-5 (70 mg) was treated with TFA/DCM (1/4, v/v, 2 mL) at room temperature for 10 minutes. The reaction mixture was evaporated to dryness under reduced pressure and the residue was purified by reverse phase preparative HPLC to give LP-S1-1 as a white powder (40 mg, TFA salt); LC-MS [ESI]: Calculated for C₄₅H₅₅N₁₅O₈ [M+H⁺]: 934.44, Found: 934.6; ¹H NM/R (400 MHz, DMSO) δ 12.88 (s, 2H), 10.18 (d, J=161.4 Hz, 3H), 8.13-7.89 (m, 3H), 7.80-7.61 (m, 2H), 7.37 (dd, J=20.5, 10.6 Hz, 4H), 6.53 (d, J=9.4 Hz, 2H), 5.97 (d, J=15.5 Hz, 1H), 5.69 (d, J=15.8 Hz, 2H), 4.95 (d, J=5.3 Hz, 2H), 4.82 (d, J=5.6 Hz, 2H), 4.76-4.37 (m, 7H), 4.08 (s, 7H), 3.98-3.50 (m, 29H), 3.37 (d, J=57.1 Hz, 8H), 2.13 (s, 6H), 1.84 (s, 2H), 1.28 (td, J=7.1, 3.3 Hz, 6H).

### Synthesis 1C-2. LP-S4-1, LP-S5-1, LP-S2-1

LP-S4-1, LP1-S5-1, and LP-S2-1 were synthesized in an analogous fashion using the same scheme and methods as described herein. The final compounds were purified by reverse phase preparative HPLC and characterized by ¹HNMR, LCMS data.

| Linker Payload | |
|---|---|
| No. | Structure |
| LP-S4-1 | 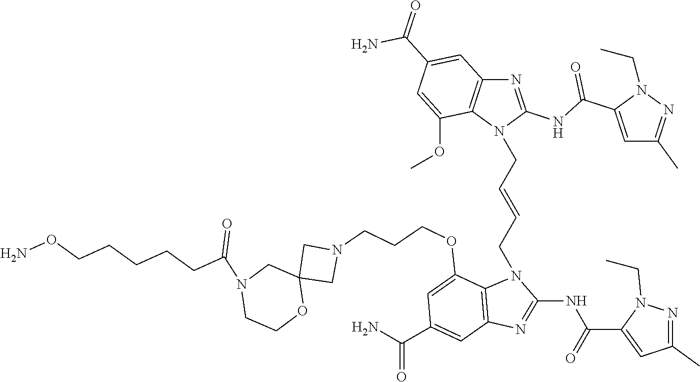 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)CCCCCON)CCO4)c21] |
| LP-S5-1 | 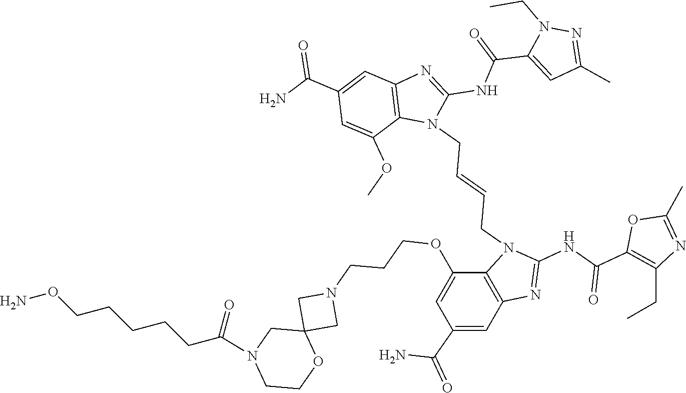 [SMILES: CCc1nc(C)oc1C(=O)Nc1nc2cc(C(N)=O)cc(OCCCN3CC4(C3)CN(C(=O)CCCCCON)CCO4)c2n1C/C=C/Cn1c(NC(=O)c2cc(C)nn2CC)nc2cc(C(N)=O)cc(OC)c21] |
| LP-S2-1 | 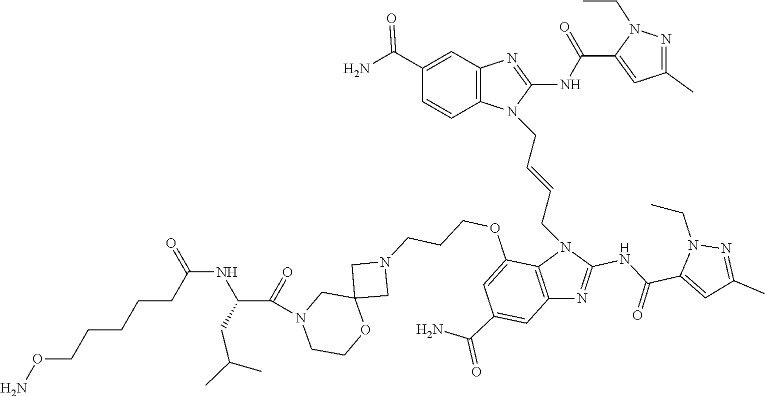 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)CCCCCON)CCO4)c21] |

### Synthesis 1C-3. LP-S1-2 (Aminooxy-PEG4-vcpAB-Compound S-1)

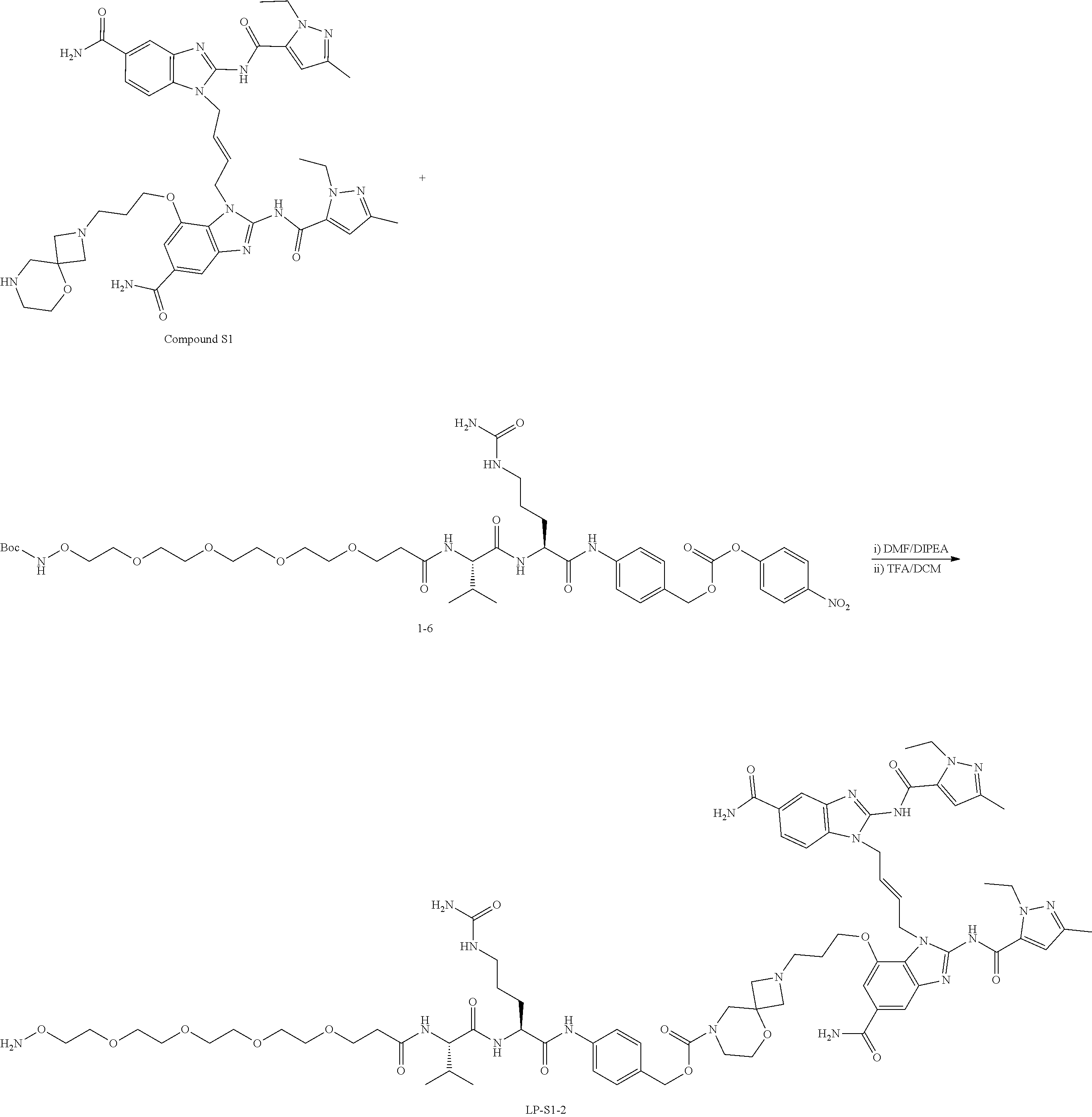 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(CNCCO4)C3)c21 ; CC(C)[C@H](NC(=O)CCOCCOCCOCCOCCONC(=O)OC(C)(C)C)C(=O)N[C@@H](CCCNC(N)=O)C(=O)Nc1ccc(COC(=O)Oc2ccc([N+](=O)[O-])cc2)cc1 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)OCc3ccc(NC(=O)[C@H](CCCNC(N)=O)NC(=O)[C@@H](NC(=O)CCOCCOCCOCCOCCON)C(C)C)cc3)CCO4)c21]

Compound S-1 (60 mg) was dissolved in anhydrous DMF (3 mL). To this solution, Boc-NO-PEG4-VC-PAB-PNP (1-6, 58 mg) was added, followed by DIPEA (0.4 mmol, 0.07 mL), and the mixture was stirred at room temperature for 3 h. LCMS showed the reaction was completed. The crude mixture was directly purified by reverse phase preparative HPLC to give Boc-protected LP-S1-2 as a white powder after lyophilization (82 mg). LC-MS [ESI]: Calculated for C₇₈H₁₀₈N₂₀O₁₉ [M+H⁺]: 1629.81, Found: 1629.9.

Boc-protected LP-S1-2 (82 mg) was treated with TFA/DCM (1/4, v/v, 2 mL) at room temperature for 15 minutes. After which, LC-MS showed the desired product formation, The reaction mixture was evaporated to dryness under reduced pressure and the residue was purified by reverse phase preparative HPLC to give LP-S1-2 as a white powder (61 mg, TFA salt). LC-MS [ESI]: Calculated for C₇₃H₁₀₀N₂₀O₁₇ [M+H⁺]: 1529.76, Found: 1529.9.

### Synthesis 1C-4. LP-S1-3 (Aminooxy-β-glucuronidase Cleavable Compound S-1)

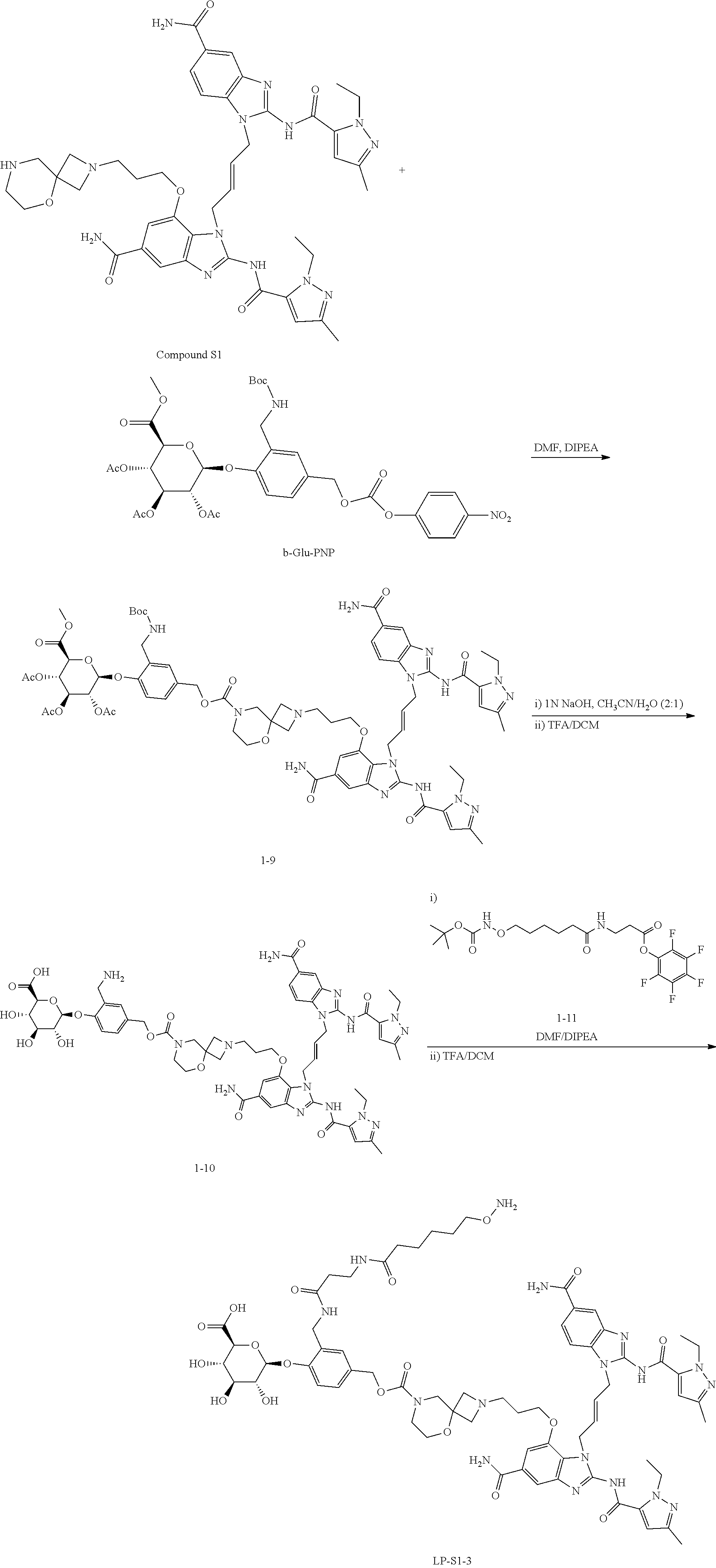

Compound 1-9: To a solution of b-Glu-PNP (46 mg, 63 mol), synthesized as described in WO 2024/006542, and Compound S-1 (TFA salt, 60 mg) in DMF (2 mL) was added DIPEA (44 L). The reaction mixture was stirred at room temperature (22° C.) for 5 hours, LCMS showed the desired product formation. The crude compound was directly by reverse phase preparative HPLC to give compound 1-9 as a white powder after lyophilization (84 mg). LC-MS [ESI]: Calculated for C₇₀H₈₅N₁₅O₂₀ [M+H⁺]: 1456.61, Found: 1456.7.

Compound 1-10: Compound 1-9 (84 mg) was dissolved in acetonitrile/water (2/1, 4 mL). To this mixture, 1N aq. NaOH (0.45 mL) was added, and the reaction was stirred at room temperature. After 3 h, 0.3 mL of 1N hydrochloric acid was added and the mixture was evaporated to dryness under reduced pressure. The residue obtained was treated with TFA/DCM (1/4, v/v, 3 mL) at room temperature for 20 mins. The mixture was diluted with toluene (30 mL) and then evaporated to dryness under reduced pressure to give the crude compound 1-10 as a TFA salt. LC-MS [ESI]: Calculated for C₅₈H₆₉N₁₅O₁₅ [M+H⁺]: 1216.51, Found: 1216.6.

LP-S1-3: The above prepared compound 1-10 was dissolved in anhydrous DMF (4 mL) and compound 1-11 (24 mg, 0.05 mol) was added, followed by DIPEA (0.1 mL). The reaction mixture was stirred at room temperature for 30 mins and purified directly by reverse phase preparative HPLC to give Boc-protected LP-S1-3 as a white powder (48 mg). Boc-protected LP-S1-3 (48 mg) was treated with TFA/DCM (1/4, v/v, 2 mL) at room temperature for 20 mins and the mixture was purified directly by reverse phase preparative HPLC to give LP-S1-3 as a white powder (43 mg); LC-MS [ESI]: Calculated for C67H85N17O18 [M+H⁺]: 1416.63, Found: 1416.7.

Intermediate I-11 was prepared following Scheme 8:

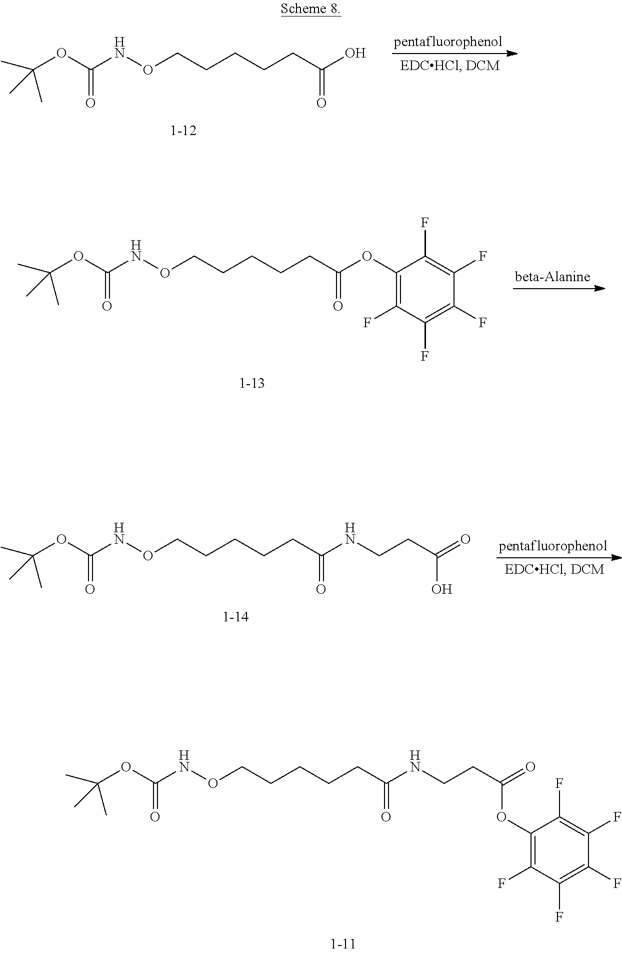 [SMILES: CC(C)(C)OC(=O)NOCCCCCC(=O)O ; CC(C)(C)OC(=O)NOCCCCCC(=O)Oc1c(F)c(F)c(F)c(F)c1F ; CC(C)(C)OC(=O)NOCCCCCC(=O)NCCC(=O)O ; CC(C)(C)OC(=O)NOCCCCCC(=O)NCCC(=O)Oc1c(F)c(F)c(F)c(F)c1F]

Compound 1-14: To a solution of compound 11-12 (247 mg, 1 mmol) in anhydrous DCM (5 mL) was added pentafluorophenol (368 mg, 2 mmol) and EDC·HCl (383 mg, 2 mmol). The mixture was stirred at room temperature for 1 h and then diluted with DCM (50 mL), and washed with 1N hydrochloric acid (30 mL) and water (30 mL). The organic layer was dried and concentrated under reduced pressure to give the crude compound 1-13, which was dissolved in acetonitrile (5 mL). To this solution, a mixture of beta-alanine (180 mg) in water (4 mL) was added, followed by DIPEA (0.7 mL, 4 mmol). After 15 min, the reaction was diluted with EtOAc (100 mL), and then HCl (1N, aq., 20 mL) was added. The organic layer was collected, washed with water (20 mL), dried, and concentrated under reduced pressure to give the crude product 1-14.

Compound 1-11: The crude compound 1-14 was dissolved in DCM (5 mL). To this solution, pentafluorophenol (368 mg, 2 mmol) and EDC·HCl (383 mg, 2 mmol) were added. The mixture was stirred at room temperature for 1 h and then diluted with DCM (50 mL), washed with 1N hydrochloric acid (30 mL) and water (30 mL). The organic layer was dried and concentrated under reduced pressure to give the crude compound 1-11 which was purified by reverse phase preparative HPLC to give the pure product 1-11 as a viscous syrup (310 mg).

### Synthesis 3E. LP-S2-2 (Aminooxy-PEG4-AAN-Compound S-2)

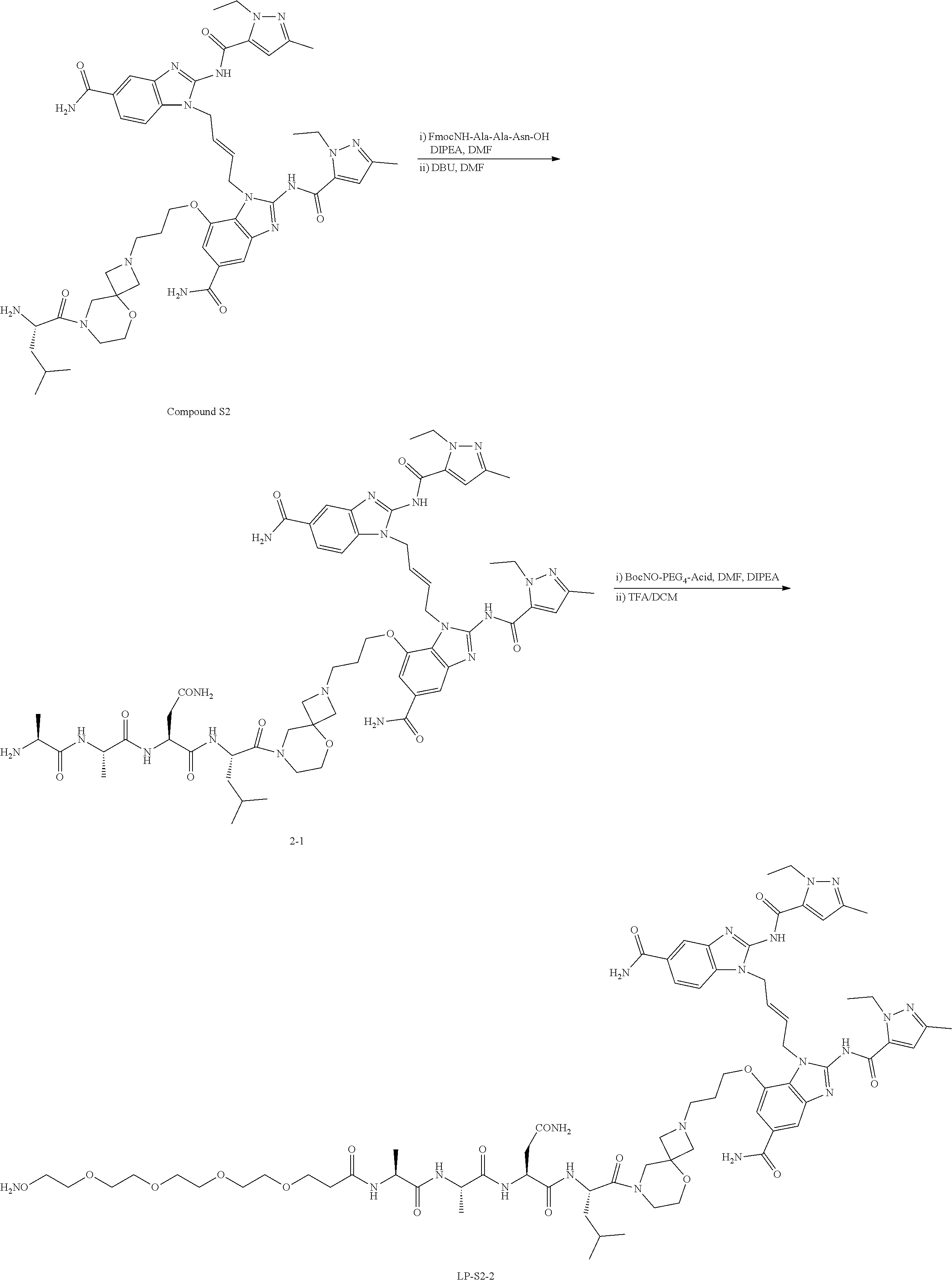

Compound 2-1: To a solution of Compound S-2 (TFA salt, 24 mg, 20 mmol) and Fmoc-Ala-Ala-Asn-OH (11 mg, 22 μmol) in DMF (1 mL) was added PyAOP (11 mg, 21 mol), followed by DIPEA (14 L). After 10 min, DBU (40 mL) was added, and the mixture was stirred at room temperature for 15 min. The crude reaction was purified directly by reverse phase preparative HPLC to give compound 2-1 (22 mg, TFA salt) as a white powder. LC-MS [ESI]: Calculated for C₅₉H₇₉N₁₉O₁₁ [M+H⁺]: 1230.62, Found: 1230.7.

LP-S2-2: To a solution of compound 2-1 (TFA salt, 22 mg, 15 mol) and BocNO-PEG4-COOH (6 mg, 16 mol) in DMF (1 mL) was added PyAOP (9 mg, 16 mol), followed by DIPEA (11 L). After 30 min, the crude reaction mixture was purified directly by reverse phase preparative HPLC to give compound to give Boc-protected LP-S2-2 (18 mg, TFA salt) as a white powder.

Boc-protected LP-S2-2 (18 mg) was treated with TFA/DCM (1/4, v/v, 1 mL) at room temperature for 20 mins and the mixture was purified directly by reverse phase preparative HPLC to give LP-2-2 as a white powder (14 mg); LC-MS [ESI]: Calculated for C₇₀H₁₀₀N₂₀O₁₇ [M+H⁺]: 1493.76, Found: 1493.9.

### Synthesis 1C-5. LP-S6-1

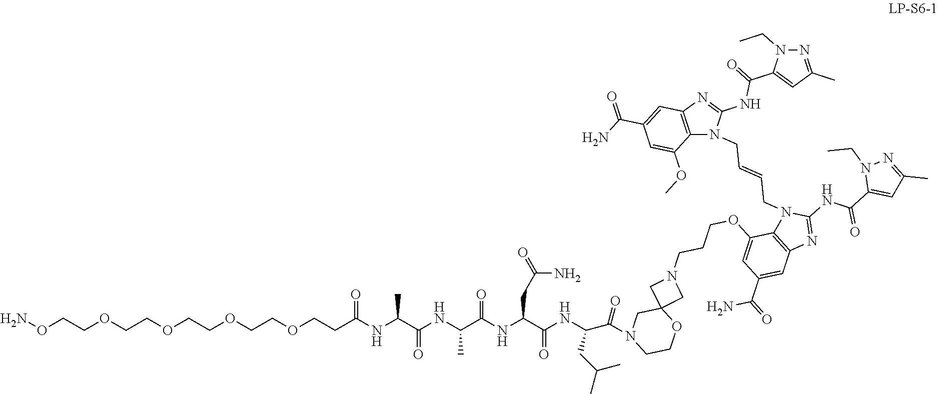 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CC4(C3)CN(C(=O)[C@H](CC(C)C)NC(=O)[C@H](CC(N)=O)NC(=O)[C@H](C)NC(=O)[C@H](C)NC(=O)CCOCCOCCOCCOCCON)CCO4)c21]

LP-S6-1 was synthesized using the same as described above starting from Compound S-6. LC-MS [ESI]: Calculated for C₇₁H₁₀₂N₂₀O₁₈ [M+H⁺]: 1523.77, Found: 1523.9.

### Synthesis 1C-6

The following linker payloads are synthesized using the methods described here from Compound S-3. The final compounds are purified by reverse phase preparative HPLC.

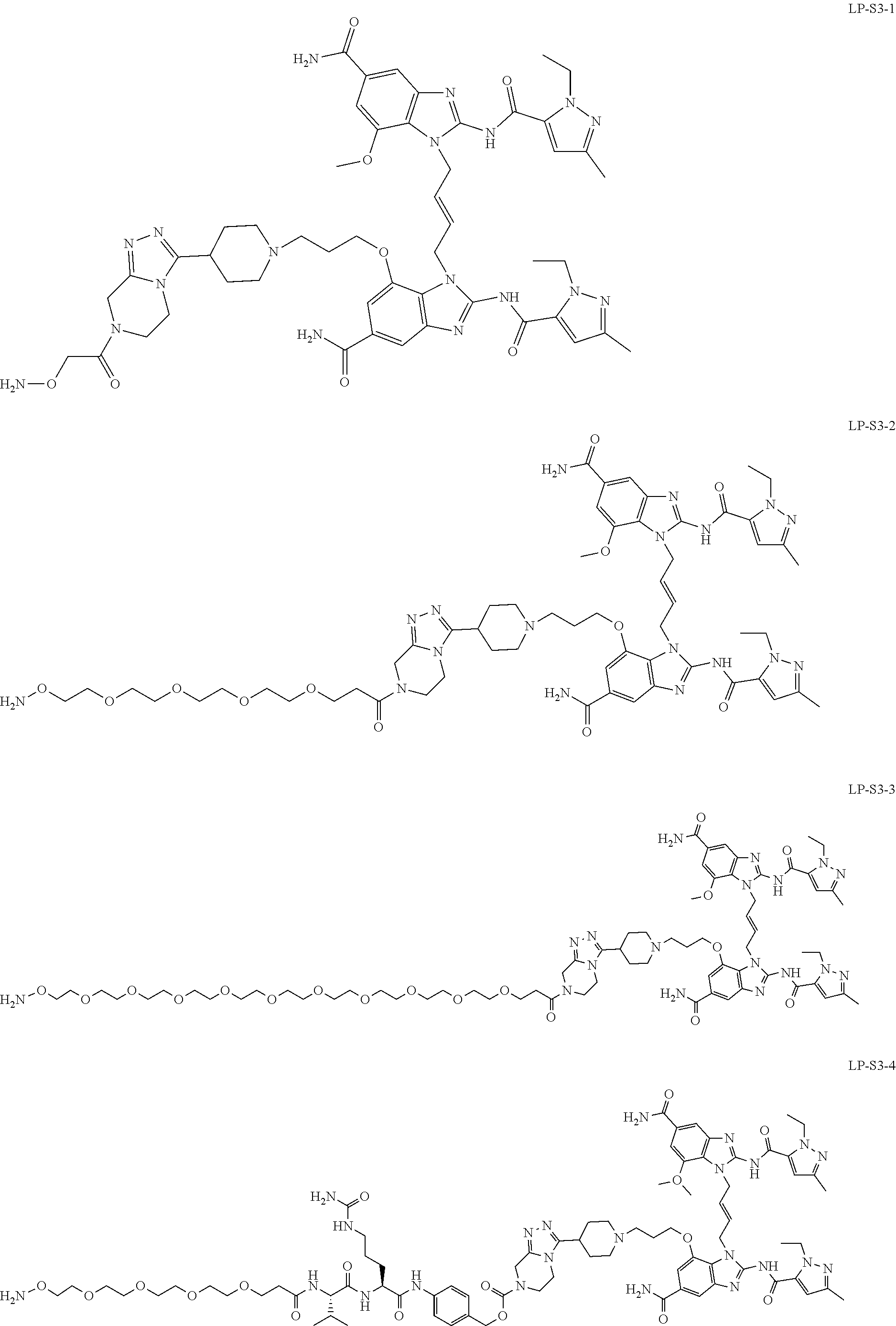

### Synthesis 1C-7. LP-S7-1

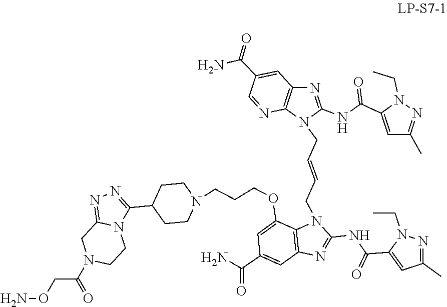 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

LP-S7-1 is synthesized using the same methods as described herein from Compound S7.

### Synthesis 1C-8. LP-S9-1

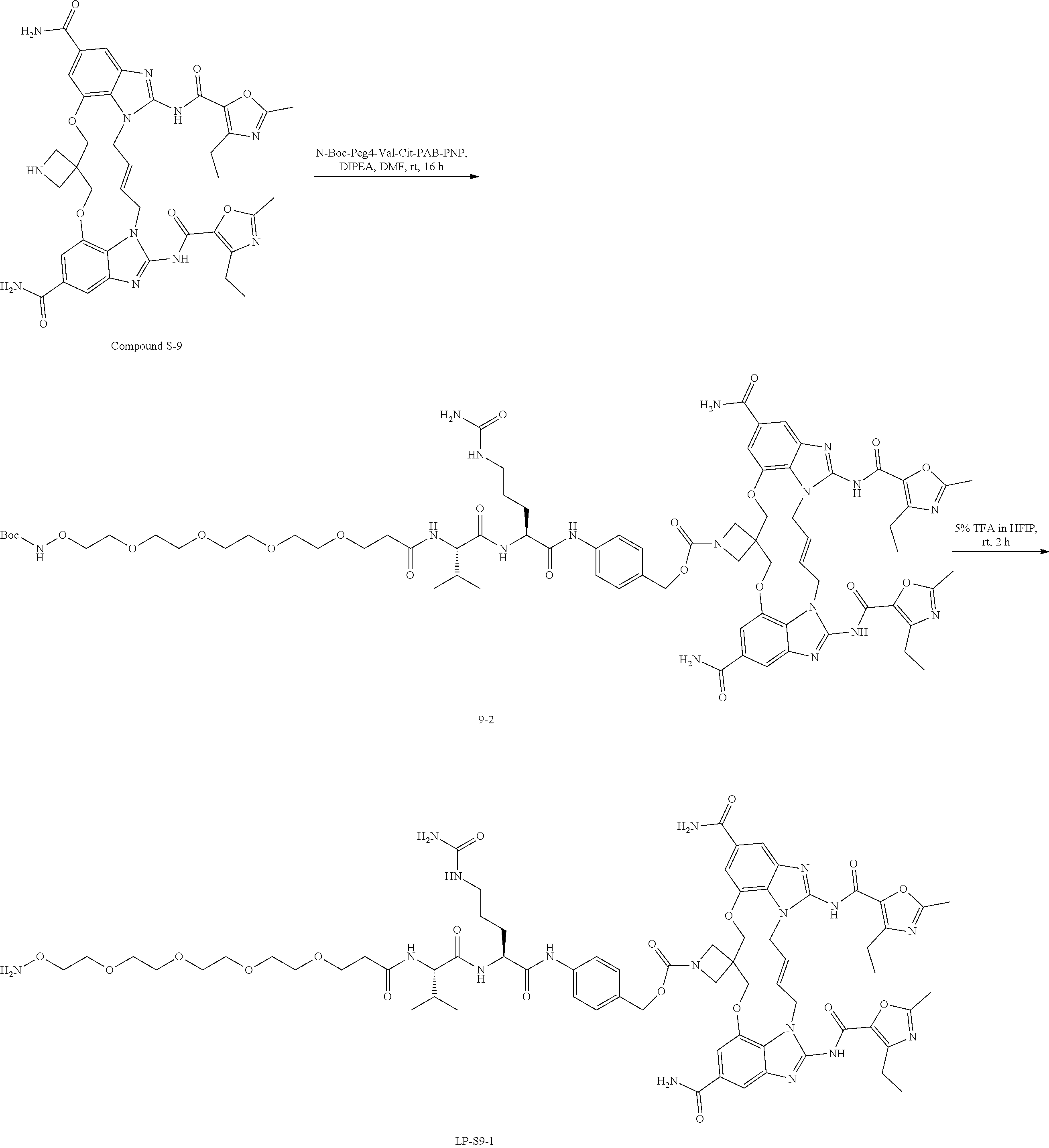 [SMILES: CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CNC1)CO3 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)[C@H](CCCNC(N)=O)NC(=O)[C@@H](NC(=O)CCOCCOCCOCCOCCONC(=O)OC(C)(C)C)C(C)C)cc2)C1 ; CCc1nc(C)oc1C(=O)Nc1nc2cc(C(N)=O)cc3c2n1C/C=C/Cn1c(NC(=O)c2oc(C)nc2CC)nc2cc(C(N)=O)cc(c21)OCC1(CO3)CN(C(=O)OCc2ccc(NC(=O)[C@H](CCCNC(N)=O)NC(=O)[C@@H](NC(=O)CCOCCOCCOCCOCCON)C(C)C)cc2)C1]

### Synthesis of [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (9-2)

To a stirred solution of (3E)-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-11,21-dicarboxamide (Compound S9, 21. mg, 0.03 mmol) in DMF (0.5000 mL) at rt was added DIPEA (0.02 mL, 0.13 mmol) followed by the addition of [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (4-nitrophenyl) carbonate (24 mg, 0.03 mmol). The solution was stirred at rt for 16 h. Reaction mixture was purified by prep-HPLC (Column: Phenomenex kinetex 5 μm C₁₈ 100 Å, 250×21.2 mm and using 5-50% ACN:1% TFA in water as eluent, Flow rate: 21.2 mL/min, run time: 30 mins to afford [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (23 mg, 0.015 mmol) as white solid.

Synthesis of [4-[[(2S)-2-[[(2S)-2-[3-[2-[2-[2-(2-aminooxyethoxy)ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (LP-S9-1): To a stirred solution of [4-[[(2S)-2-[[(2S)-2-[3-[2-[2-[2-[2-(tert-butoxycarbonylamino)oxyethoxy]ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate (23. mg, 0.01 mmol) in HFIP (0.5 mL) at rt was added 5% TFA in HFIP (1.13 mL, 0.74 mmol). The solution was stirred at rt for 2 h. Reaction mixture was concentrated under reduced pressure and crude was purified by reverse phase preparative HPLC (Column: Phenomenex kinetex 5 μm C18 100 Å, 250×21.2 mm and using 5-50% ACN: 1% TFA in water as eluent, Flow rate: 21.2 mL/min, run time: 30 mins to give [4-[[(2S)-2-[[(2S)-2-[3-[2-[2-[2-(2-aminooxyethoxy)ethoxy]ethoxy]ethoxy]propanoylamino]-3-methyl-butanoyl]amino]-5-ureido-pentanoyl]amino]phenyl]methyl (3E)-11,21-dicarbamoyl-7,25-bis[(4-ethyl-2-methyl-oxazole-5-carbonyl)amino]spiro[14,18-dioxa-1,6,8,24-tetrazapentacyclo[17.6.1.16,9.023,26.013,27]heptacosa-3,7,9,11,13(27),19(26),20,22,24-nonaene-16,3′-azetidine]-1′-carboxylate LP-S9-1 (14.1 mg) as white solid. ¹H NMR (400 MHz, DMSO) δ 10.03 (s, 1H), 8.14 (d, J=7.4 Hz, 1H), 8.00 (s, 2H), 7.89 (d, J=8.7 Hz, 1H), 7.84 (s, 2H), 7.77 (d, J=1.2 Hz, 2H), 7.61 (d, J=8.5 Hz, 2H), 7.45 (s, 2H), 7.35 (d, J=8.4 Hz, 2H), 6.03 (s, 1H), 5.33 (s, 2H), 5.05 (s, 2H), 4.80 (s, 4H), 4.55 (s, 4H), 4.39 (q, J=7.5 Hz, 1H), 4.24 (dd, J=8.7, 6.6 Hz, 1H), 4.11-4.05 (m, 2H), 3.97 (d, J=16.7 Hz, 4H), 3.66-3.62 (m, 6H), 2.77 (q, J=7.5 Hz, 4H), 2.37 (s, 6H), 1.98 (dt, J=13.5, 6.8 Hz, 1H), 1.69 (t, J=7.7 Hz, 1H), 1.60 (dd, J=9.3, 4.9 Hz, 1H), 1.41 (dt, J=25.0, 5.5 Hz, 3H), 0.96 (t, J=7.5 Hz, 6H), 0.85 (dd, J=13.2, 6.8 Hz, 6H).

### Synthesis 1C-9. LP-S3-5 (Aminooxy-β-glucuronidase Cleavable-Compound S3)

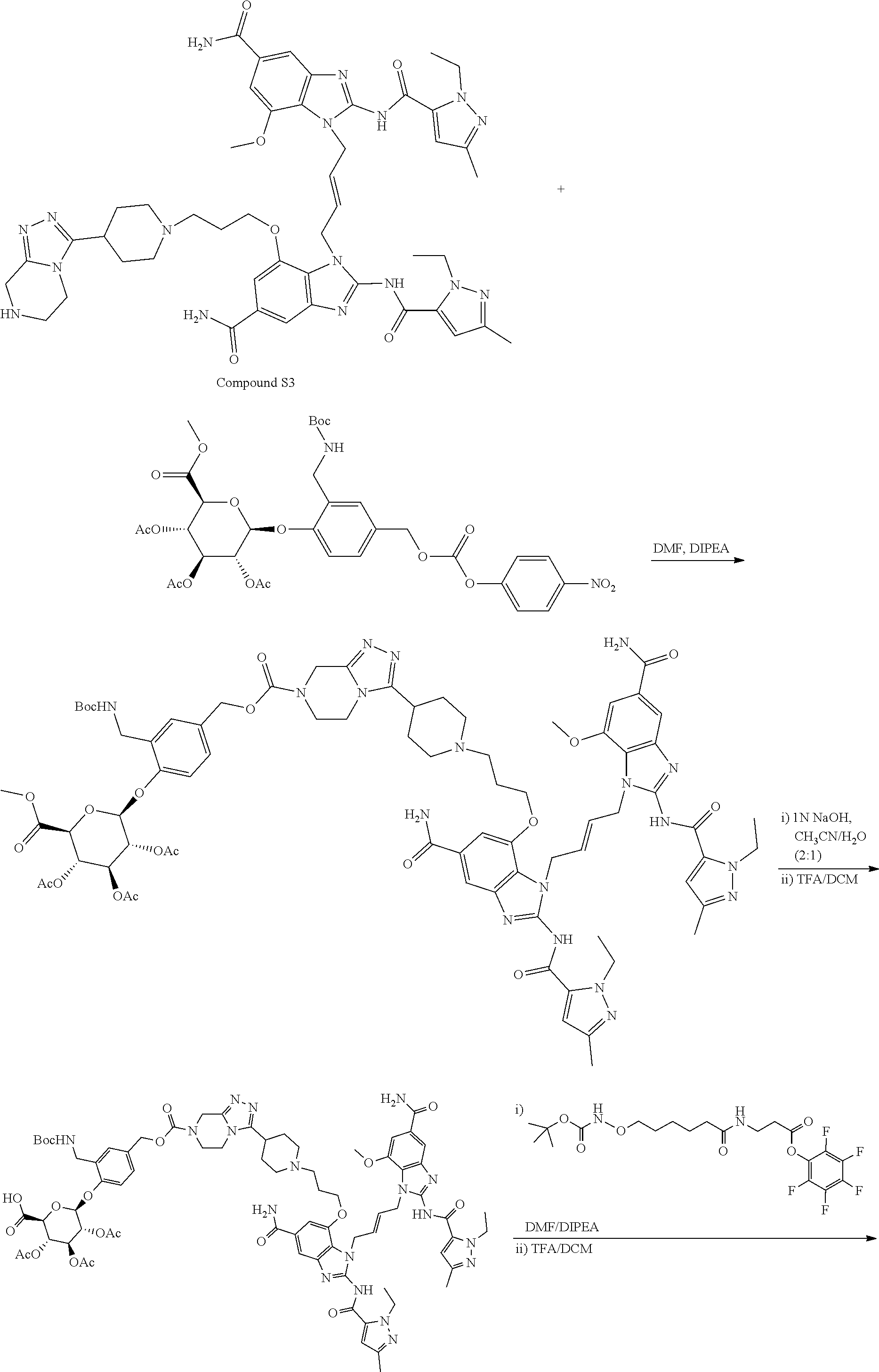

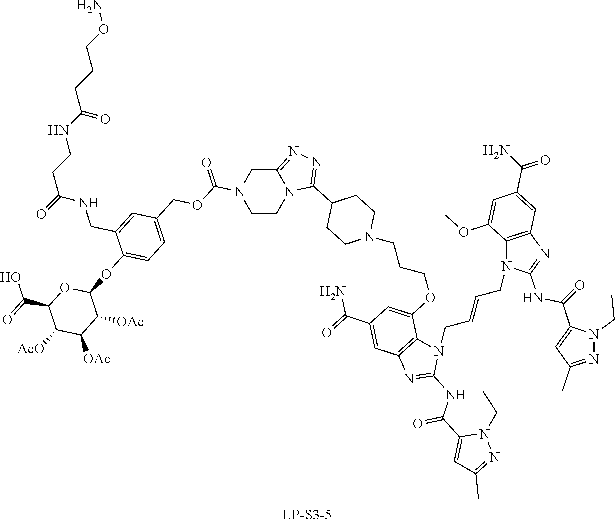 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)OCc4ccc(O[C@@H]6O[C@H](C(=O)O)[C@@H](OC(C)=O)[C@H](OC(C)=O)[C@H]6OC(C)=O)c(CNC(=O)CCNC(=O)CCCON)c4)C5)CC3)c21]

LP-S3-5 was synthesized using the same methods in an analogous fashion as described herein. LC-MS [ESI]: Calculated for C₇₂H₉₃N₂₀O₁₈ [M+H⁺]: 1525.69, Found: 1529.7. ¹H NMR (400 MHz, DMSO) δ 8.21 (t, J=6.1 Hz, 1H), 7.99 (d, J=17.6 Hz, 2H), 7.83 (t, J=5.6 Hz, 1H), 7.67 (d, J=6.0 Hz, 2H), 7.39 (s, 2H), 7.32 (d, J=11.2 Hz, 3H), 7.21 (s, 1H), 7.08 (d, J=8.6 Hz, 1H), 6.53 (d, J=7.2 Hz, 2H), 5.77 (q, J=15.5 Hz, 3H), 5.08 (s, 2H), 4.94 (dd, J=21.2, 10.0 Hz, 6H), 4.74 (s, 3H), 4.58-4.45 (m, 5H), 4.38-4.22 (m, 3H), 4.00 (s, 5H), 3.94-3.84 (m, 7H), 3.71 (s, 5H), 3.65 (s, 12H), 3.43 (d, J=9.0 Hz, 4H), 3.34-3.31 (m, 2H), 3.24 (t, J=6.4 Hz, 3H), 3.10 (s, 2H), 2.97-2.82 (m, 3H), 2.31 (t, J=7.6 Hz, 3H), 2.15-2.00 (m, 12H), 1.91 (d, J=9.9 Hz, 4H), 1.55 (t, J=7.6 Hz, 2H), 1.48 (p, J=7.0 Hz, 2H), 1.33-1.21 (m, 9H).

### Synthesis 1C-10. Non-Cleavable Compound 10 Aminooxy LP-S10-1

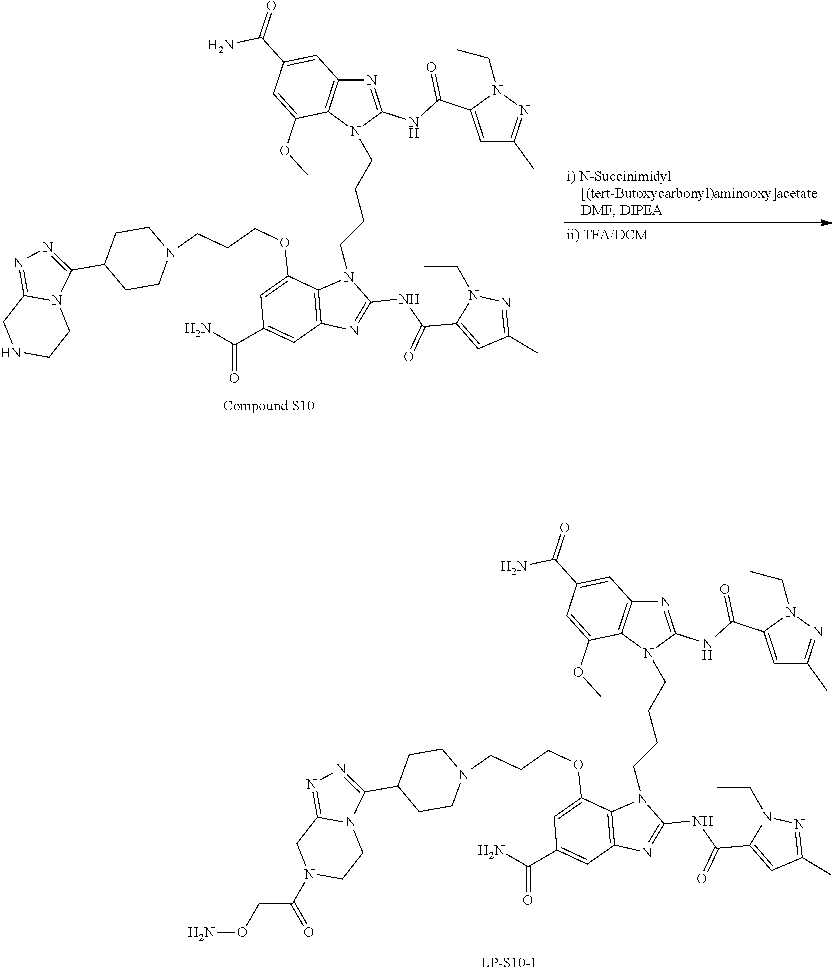 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCNC5)CC3)c21 ; CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCC(c4nnc5n4CCN(C(=O)CON)C5)CC3)c21]

LP-S10-1 was synthesized using the same methods described herein in an analogous fashion. LC-MS [ESI]: Calculated for C₅₀H₆₅N₁₈O₈ [M+H⁺]: 1045.52, Found: 1045.6. ¹H NMR (400 MHz, DMSO) δ 12.81 (s, 2H), 9.25 (s, 1H), 8.01 (s, 2H), 7.55 (s, 2H), 7.40 (s, 2H), 7.32 (s, 1H), 6.61 (s, 1H), 6.52 (s, 10H), 4.79 (s, 2H), 4.61-4.54 (m, 7H), 4.35 (s, 3H), 4.16 (s, 3H), 4.03 (s, 1H), 3.95 (s, 2H), 3.89 (s, 1H), 3.84 (d, J=9.0 Hz, 4H), 3.14 (s, 1H), 2.92 (s, 3H), 2.61 (s, 2H), 2.54 (s, 2H), 2.11 (dd, J=6.2, 2.8 Hz, 6H), 2.05 (s, 6H), 1.86 (s, 5H), 1.32 (q, J=7.1 Hz, 6H).

### Synthesis 1C-11. LP-S2-3

### Step 1. 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate

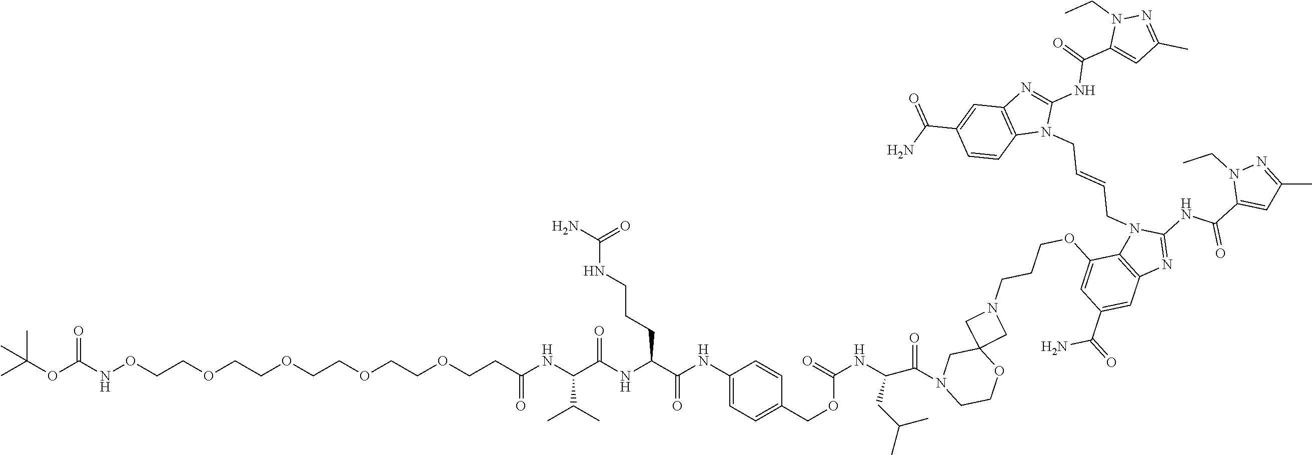 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)OCc3ccc(NC(=O)[C@H](CCCNC(N)=O)NC(=O)[C@@H](NC(=O)CCOCCOCCOCCOCCONC(=O)OC(C)(C)C)C(C)C)cc3)CCO4)c21]

To a DMF (1 mL) solution of (E)-7-(3-(8-(L-leucyl)-5-oxa-2,8-diazaspiro[3.5]nonan-2-yl)propoxy)-1-(4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazole-5-carboxamide (Compound S-2, 77 mg, 0.08 mmol) and DIPEA (0.138 mL, 0.80 mmol) was added DMF (1 mL) solution of tert-butyl (((6S,9S)-1-amino-9-isopropyl-6-((4-((((4-nitrophenoxy)carbonyl)oxy)methyl)phenyl)carbamoyl)-1,8,11-trioxo-14,17,20,23-tetraoxa-2,7,10-triazapentacosan-25-yl)oxy)carbamate (79.0 mg, 0.087 mmol) dropwise at 0° C., reaction mixture was stirred at 0° C. for 15 mins, then gradually warmed to room temperature and stirred overnight. The reaction solution was purified by reverse phase preparative HPLC (acetonitrile/water+0.1% formic acid) to afford 4-((23 S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate as a white solid (82 mg). LCMS (ESI) m/z 1742.9 (M+H).

### Step 2: 4-((17S,20S)-1-(aminooxy)-17-isopropyl-15,18-dioxo-20-(3-ureidopropyl)-3,6,9,12-tetraoxa-16,19-diazahenicosan-21-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate

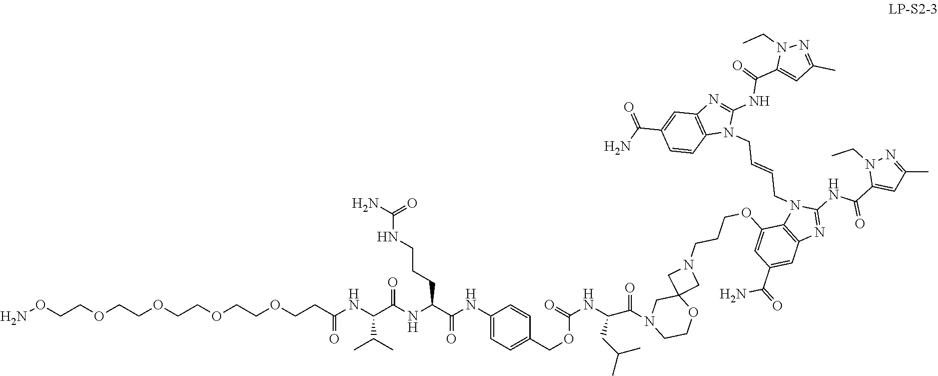 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CC4(C3)CN(C(=O)[C@H](CC(C)C)NC(=O)OCc3ccc(NC(=O)[C@H](CCCNC(N)=O)NC(=O)[C@@H](NC(=O)CCOCCOCCOCCOCCON)C(C)C)cc3)CCO4)c21]

A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate (82.0 mg, 0.047 mmol) in 2 mL of 5% (v/v) trifluoracetic acid in 1,1,1,3,3,3-Hexafluoroisopropanol (HFIP) was stirred at room temperature for 1 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl ((S)-1-(2-(3-((5-carbamoyl-1-((E)-4-(5-carbamoyl-2-(1-ethyl-3-methyl-1H-pyrazole-5-carboxamido)-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)-5-oxa-2,8-diazaspiro[3.5]nonan-8-yl)-4-methyl-1-oxopentan-2-yl)carbamate (LP-S2-3) as white solid, 57 mg, 74%). LCMS (ESI) m/z 1642.8 (M+H). ¹H NMR (400 MHz, DMSO) δ 12.84 (s, 2H), 10.40 (s, 3H), 7.96 (d, J=16.1 Hz, 3H), 7.73-7.64 (m, 2H), 7.59 (d, J=8.2 Hz, 3H), 7.38 (d, J=8.6 Hz, 3H), 7.27 (d, J=8.2 Hz, 2H), 6.53 (d, J=12.8 Hz, 2H), 5.98 (d, J=17.3 Hz, 2H), 4.94 (d, J=13.6 Hz, 4H), 4.53 (d, J=7.3 Hz, 4H), 4.41-4.35 (m, 3H), 4.09-4.03 (m, 3H), 3.85-3.76 (m, 56H), 3.61 (dt, J=17.1, 4.8 Hz, 5H), 3.55-3.46 (m, 12H), 3.17 (s, 14H), 2.10 (d, J=17.0 Hz, 11H), 1.82 (s, 2H), 1.27 (td, J=7.1, 2.9 Hz, 6H), 0.89-0.79 (m, 12H).

### Synthesis 1C-12. LP-S-11-1

### Step 1. 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate

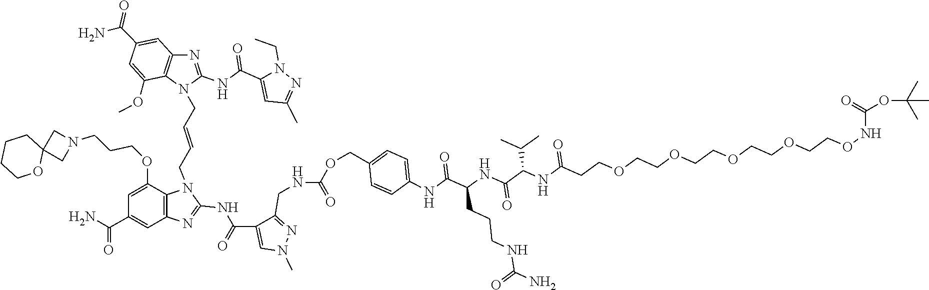 [SMILES: CCn1nc(C)cc1C(=O)Nc1nc2cc(C(N)=O)cc(OC)c2n1C/C=C/Cn1c(NC(=O)c2cn(C)nc2CNC(=O)OCc2ccc(NC(=O)[C@H](CCCNC(N)=O)NC(=O)[C@@H](NC(=O)CCOCCOCCOCCOCCONC(=O)OC(C)(C)C)C(C)C)cc2)nc2cc(C(N)=O)cc(OCCCN3CC4(CCCCO4)C3)c21]

To a DMF (1 mL) solution of (E)-7-(3-(5-oxa-2-azaspiro[3.5]nonan-2-yl)propoxy)-2-(3-(aminomethyl)-1-methyl-1H-pyrazole-4-carboxamido)-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazole-5-carboxamide (Compound S-11, 20 mg) and DIPEA (0.039 mL, 0.22 mmol) was added DMF (0.5 mL) solution of tert-butyl (((6S,9S)-1-amino-9-isopropyl-6((4((((4nitrophenoxy)carbonyl)oxy)methyl)phenyl)carbamoyl)-1,8,11-trioxo-14,17,20,23-tetraoxa-2,7,10-triazapentacosan-25-yl)oxy)carbamate (20.4 mg, 0.022 mmol) dropwise at 0° C., reaction mixture was stirred at 0° C. for 15 mins, then gradually warmed to room temperature and stirred overnight. The reaction solution was purified by reverse phase preparative HPLC (acetonitrile/water+0.1% formic acid) to afford 4-((23 S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate as a white solid (24 mg). LCMS (ESI) m/z 1659.8 (M+H).

### Synthesis of Compound S-11

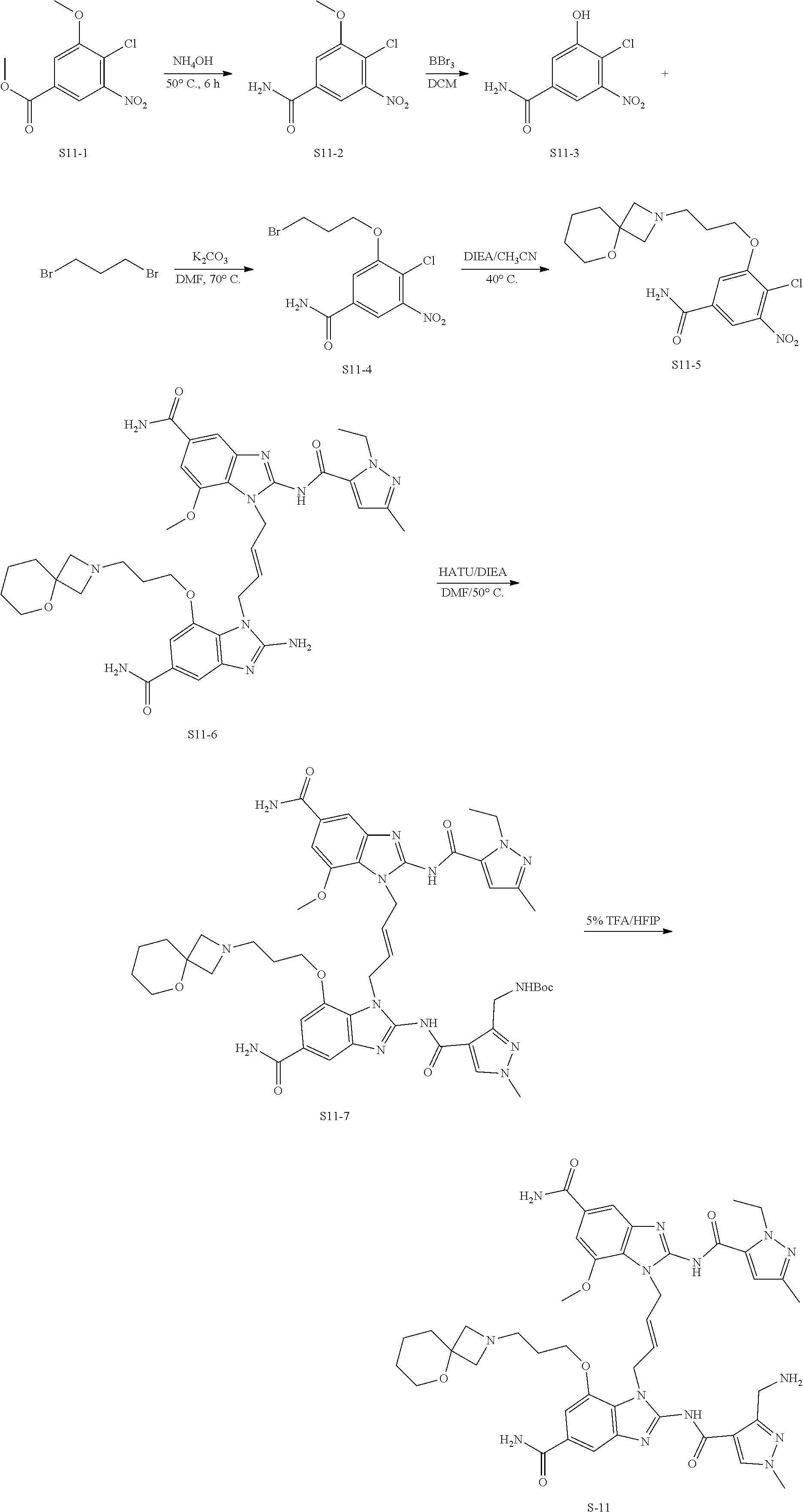

### Synthesis of 4-chloro-3-methoxy-5-nitrobenzamide (S11-2)

A mixture of methyl 4-chloro-3-methoxy-5-nitrobenzoate (S11-1) (13.7 g, 55.8 mmol) and NH₄OH (200 mL) was heated to 50° C. in pressure vessel and the reaction was stirred for 6 hours. The resulting solid was filtered and dried under vacuum to afford 4-chloro-3-methoxy-5-nitrobenzamide (S11-2, 9.45 g, 74%) as a yellow solid. LCMS (ESI) m/z 231 (M+H).

### Synthesis of 4-chloro-3-hydroxy-5-nitrobenzamide (S11-3)

To a stirred solution of 4-chloro-3-hydroxy-5-nitrobenzamide (S11-2) (5 g, 21.7 mmol) in DCM (80 mL) under N₂ was added neat BBr₃. The mixture was stirred at room temperature for 16 hours, then was poured into ice (100 g) and stirred for an hour, the resulting solid was filtered and washed with cold water and heptane and dried under vacuum to afford 4-chloro-3-hydroxy-5-nitrobenzamide (S11-3) (4.60 g, 98%) as a light brown solid. LCMS (ESI) m/z 217.08 (M+H).

### Synthesis of 3-(3-bromopropoxy)-4-chloro-5-nitrobenzamide (S11-4)

To a stirred solution of 3-(3-bromopropoxy)-4-chloro-5-nitrobenzamide (S11-3) (5 g, 23.1 mmol) in DMF (100 mL) was added 1,3-dibromopropane (10 mL, 97 mmol) and K₂CO₃ (19.8 g, 143 mmol). The mixture was heated to 70° C. and stirred for 1.5 hours. The reaction mixture was poured into ice cold water and extracted with ethyl acetate, combined organic layers were washed with brine and dried with Na₂SO₄ followed by filtration and evaporation to provide crude which was purified by flash chromatography (ethyl acetate:DCM=1:4) to give 3-(3-bromopropoxy)-4-chloro-5-nitrobenzamide (S11-4) (3.10 g, 40%). LCMS (ESI) m/z 338.08 (M+H).

### Synthesis of 3-(3-(5-oxa-2-azaspiro[3.5]nonan-2-yl)propoxy)-4-chloro-5-nitrobenzamide (S11-5)

To a stirred solution of 3-(3-bromopropoxy)-4-chloro-5-nitrobenzamide (S11-4) (9.53 g, 28.2 mmol) in CH₃CN (100 mL) was added 5-oxa-2-azaspiro[3.5]nonane hydrochloride (5 g, 25.4 mmol) and DIPEA (12.3 mL, 70.6 mmol). The mixture was heated to 40° C. and stirred for 2 hours. Reaction mixture was diluted with water and extracted with ethyl acetate, the combined organic layers were washed with brine, dried over Na₂SO₄, filtered and evaporated to provide crude which was purified by flash chromatography (3% MeOH/1% Et₃N/DCM) to afford 3-(3-(5-oxa-2-azaspiro[3.5]nonan-2-yl)propoxy)-4-chloro-5-nitrobenzamide (S11-5) (3.90 g, 36%). LCMS (ESI) m/z 384.1 (M+H). ¹H NMR (400 MHz, DMSO) δ 8.28 (s, 1H), 8.04 (d, J=1.7 Hz, 1H), 7.87 (d, J=1.8 Hz, 1H), 7.76 (s, 1H), 4.25 (t, J=6.3 Hz, 2H), 4.08 (dt, J=7.8, 5.2 Hz, 1H), 3.47 (t, J=5.2 Hz, 2H), 3.37 (q, J=7.3 Hz, 1H), 3.27 (d, J=7.2 Hz, 2H), 3.18 (d, J=5.2 Hz, 3H), 2.83 (d, J=7.0 Hz, 2H), 2.60 (t, J=6.8 Hz, 2H), 1.81 (p, J=6.6 Hz, 2H), 1.66 (dd, J=7.5, 4.2 Hz, 2H), 1.57 (tq, J=8.6, 4.4 Hz, 2H), 1.41 (p, J=5.5 Hz, 2H), 1.24 (t, J=7.2 Hz, 1H), 1.07 (s, 1H).

### Synthesis of tert-butyl (E)-((4-((7-(3-(5-oxa-2-azaspiro[3.5]nonan-2-yl)propoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate (S11-7)

To a stirred solution of (E)-7-(3-(5-oxa-2-azaspiro[3.5]nonan-2-yl)propoxy)-2-amino-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazole-5-carboxamide (S11-6, 50 mg, 0.066 mmol) in DMF (1 mL) was added 3-[(tert-butoxycarbonylamino)methyl]-1-methyl-pyrazole-4-carboxylic acid (25.4 mg, 0.099 mmol), HATU (38.0 mg, 0.099 mmol) and DIPEA (0.035 mL, 0.199 mmol). The mixture was heated to 60° C. and stirred for 10 hours. The reaction solution was purified by reverse phase preparative HPLC (acetonitrile/water+0.1% formic acid) to afford tert-butyl (E)-((4-((7-(3-(5-oxa-2-azaspiro[3.5]nonan-2-yl)propoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate (S11-7) (47 mg, 72%). LCMS (ESI) m/z 992.08 (M+H).

### Synthesis of S-11 ((E)-7-(3-(5-oxa-2-azaspiro[3.5]nonan-2-yl)propoxy)-2-(3-(aminomethyl)-1-methyl-1H-pyrazole-4-carboxamido)-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazole-5-carboxamide)

To a stirred solution of tert-butyl (E)-((4-((7-(3-(5-oxa-2-azaspiro[3.5]nonan-2-yl)propoxy)-5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate (S11-7) (36 mg, 0.036 mmol) was added 5% TFA in HFIP (1 mL), reaction mixture was stirred at RT for one hour. The solvent was evaporated and residue was dissolved in DMSO for purification by reverse phase preparative HPLC (acetonitrile/water+0.1% TFA) to afford S-11 (30 mg, 93%). LCMS (ESI) m/z 891.6 (M+H). ¹H NMR (400 MHz, DMSO) δ 10.20 (s, 1H), 9.80 (s, 1H), 8.32 (s, 3H), 8.20 (s, 1H), 7.97 (d, J=11.7 Hz, 2H), 7.66 (d, J=12.7 Hz, 2H), 7.41 (d, J=7.6 Hz, 2H), 7.33-7.25 (m, 2H), 6.53 (s, 1H), 5.80 (s, 2H), 4.95 (s, 2H), 4.89 (s, 1H), 4.54 (d, J=7.4 Hz, 2H), 4.25 (d, J=5.9 Hz, 2H), 4.09 (dd, J=11.2, 6.2 Hz, 1H), 3.99 (d, J=8.3 Hz, 3H), 3.83 (s, 3H), 3.63 (d, J=2.8 Hz, 3H), 3.26-3.15 (m, 2H), 2.12 (s, 3H), 1.76 (s, 1H), 1.55 (s, 1H), 1.44 (s, 2H), 1.39 (s, 1H), 1.29 (t, J=7.1 Hz, 3H).

Step 2: 4-((17S,20S)-1-(aminooxy)-17-isopropyl-15,18-dioxo-20-(3-ureidopropyl)-3,6,9,12-tetraoxa-16,19-diazahenicosan-21-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate:

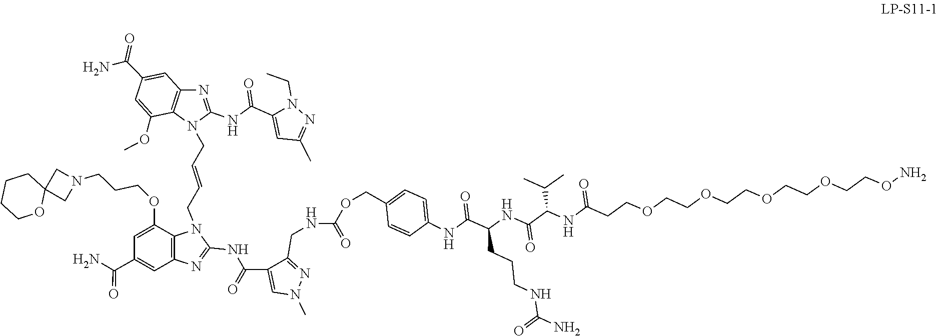 [SMILES: CCn1nc(C)cc1C(=O)Nc1nc2cc(C(N)=O)cc(OC)c2n1C/C=C/Cn1c(NC(=O)c2cn(C)nc2CNC(=O)OCc2ccc(NC(=O)[C@H](CCCNC(N)=O)NC(=O)[C@@H](NC(=O)CCOCCOCCOCCOCCON)C(C)C)cc2)nc2cc(C(N)=O)cc(OCCCN3CC4(CCCCO4)C3)c21]

A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate (24.0 mg, 0.022 mmol) in 2 mL of 5% (v/v) trifluoracetic acid in 1,1,1,3,3,3-Hexafluoroisopropanol (HFIP) was stirred at room temperature for 1 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl ((4-((7-(3-(5-oxa-2-azaspiro[3.5]nonan-2-yl)propoxy)-5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-1H-benzo[d]imidazol-2-yl)carbamoyl)-1-methyl-1H-pyrazol-3-yl)methyl)carbamate (LP-S11-1) as its TFA salt. (white solid, 13.0 mg, 58%). LCMS (ESI) m/z 1559.8 (M+H). ¹H NMR (400 MHz, DMSO) δ 12.88 (s, 1H), 10.00 (s, 1H), 8.16-8.08 (m, 2H), 7.99 (s, 1H), 7.96-7.85 (m, 2H), 7.66 (s, 1H), 7.59 (d, J=8.2 Hz, 2H), 7.41 (s, 1H), 7.36 (s, 1H), 7.27 (d, J=16.6 Hz, 5H), 6.54 (s, 1H), 5.99 (s, 1H), 5.81 (s, 2H), 5.43 (s, 2H), 4.95 (s, 3H), 4.88 (s, 2H), 4.53 (s, 4H), 4.28-4.20 (m, 1H), 4.05 (s, 2H), 3.93 (s, 3H), 3.79 (s, 2H), 3.67-3.57 (m, 1H), 3.55-3.46 (m, 11H), 3.20 (s, 2H), 2.95 (s, 1H), 2.39 (dd, J=13.6, 6.8 Hz, 1H), 2.09 (d, J=10.4 Hz, 3H), 1.65 (s, 6H), 1.41 (s, 7H), 1.33-1.22 (m, 3H), 0.85 (dd, J=13.2, 6.8 Hz, 6H).

### Synthesis 1C-13. LP-S12-1

### Step 1. tert-butyl (S,E)-((6-((1-(3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7(8H)-yl)-4-methyl-1-oxopentan-2-yl)amino)-6-oxohexyl)oxy)carbamate

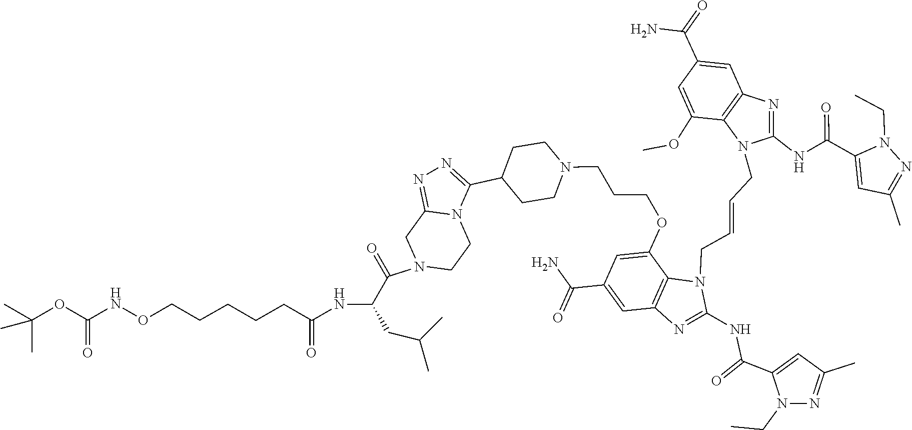 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCONC(=O)OC(C)(C)C)C5)CC3)c21]

A solution of 6-(tert-butoxycarbonylamino)oxyhexanoic acid (47.0 mg, 0.189 mmol), HBTU (72.0 mg, 0.189 mmol) and DIPEA (0.11 mL, 0.63 mmol in DMF (1.0 mL) was stirred for 10 min and the mixture was then added dropwise to DMF (1.0 mL) solution of (S,E)-7-(3-(4-(7-(2-(12-azaneyl)-4-methylpentanoyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide HCl salt (Compound S-12, 141 mg, 0.126 mmol). The reaction mixture was stirred at room temperature for 2 hrs, then purified by reverse phase preparative HPLC (acetonitrile/water+0.1% formic acid) to afford tert-butyl (S,E)-((6-((1-(3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7(8H)-yl)-4-methyl-1-oxopentan-2-yl)amino)-6-oxohexyl)oxy)carbamate as a white solid (77 mg, 46%). LCMS (ESI) m/z 1312.7 (M+H).

### Step 2. (E)-7-(3-(4-(7-((6-(aminooxy)hexanoyl)-L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide

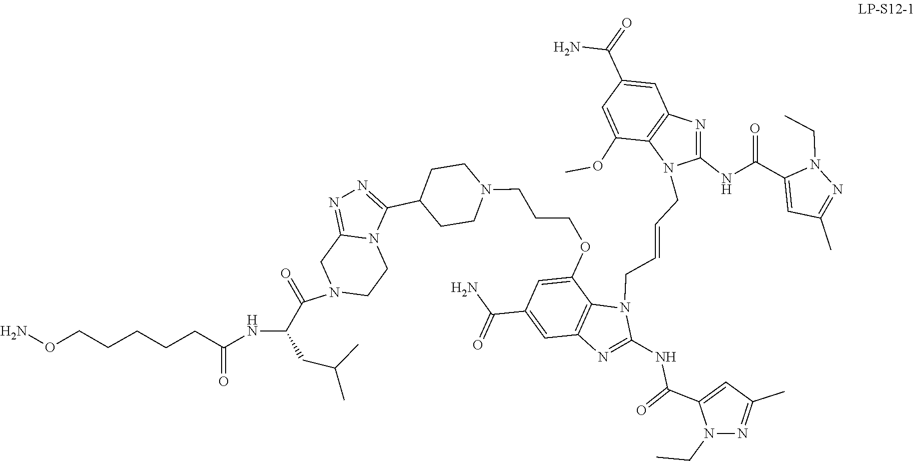 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)[C@H](CC(C)C)NC(=O)CCCCCON)C5)CC3)c21]

A mixture of tert-butyl (S,E)-((6-((1-(3-(1-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7(8H)-yl)-4-methyl-1-oxopentan-2-yl)amino)-6-oxohexyl)oxy)carbamate (77.0 mg, 0.058 mmol) in 2 mL of 5% (v/v) trifluoracetic acid in 1,1,1,3,3,3-Hexafluoroisopropanol (HFIP) was stirred at room temperature for 1 hour. The reaction mixture was concentrated. The residue was diluted with DMSO (2 mL) and purified by reverse phase preparative HPLC (acetonitrile/water+0.1% TFA) to afford (E)-7-(3-(4-(7-((6-(aminooxy)hexanoyl)-L-leucyl)-5,6,7,8-tetrahydro-[1,2,4]triazolo[4,3-a]pyrazin-3-yl)piperidin-1-yl)propoxy)-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazole-5-carboxamide (LP-S12-1) as its TFA salt. ¹H NMR (400 MHz, DMSO) δ 7.97 (s, OH), 7.68 (d, J=4.5 Hz, 1H), 7.40 (s, 1H), 7.32 (d, J=12.4 Hz, 1H), 6.54 (d, J=6.9 Hz, 1H), 4.92 (d, J=13.2 Hz, 2H), 4.53 (dd, J=15.5, 7.7 Hz, 2H), 4.02 (s, 3H), 3.97-3.87 (m, 1H), 3.71 (s, 1H), 3.47 (s, 5H), 3.11 (s, 1H), 2.89 (s, 1H), 2.12 (d, J=12.0 Hz, 3H), 1.92 (s, 2H), 1.55 (s, 3H), 1.28 (dt, J=15.9, 7.0 Hz, 3H), 0.94-0.87 (m, 3H)

### Synthesis 1C-14. LP-S-13-1

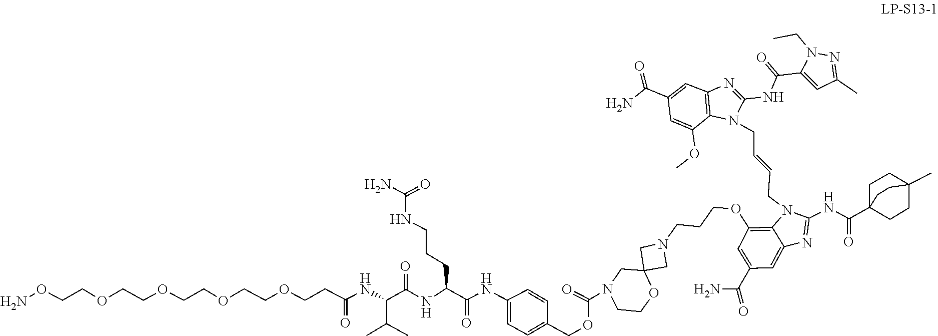 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)OCc3ccc(NC(=O)[C@H](CCCNC(N)=O)NC(=O)[C@@H](NC(=O)CCOCCOCCOCCOCCON)C(C)C)cc3)CCO4)c21]

LP-S13-1 was synthesized and purified by reverse phase HPLC using the same methods as described above. Analytical data, LC-MS [ESI]: Calculated for C₇₇H₁₀₉N₁₈O₁₈ [M+H⁺]: 1573.81, observed 1573.83. ¹H NMR (400 MHz, DMSO) δ 12.83 (s, 1H), 10.45 (s, 4H), 10.02 (s, 1H), 8.14 (d, J=7.6 Hz, 1H), 7.98 (s, 2H), 7.88 (d, J=8.6 Hz, 1H), 7.60 (d, J=8.3 Hz, 2H), 7.35-7.28 (m, 5H), 6.53 (s, 1H), 6.02 (s, 1H), 5.76 (d, J=15.4 Hz, 1H), 5.59 (s, 1H), 5.04 (s, 2H), 4.82-4.77 (m, 2H), 4.51 (q, J=7.2 Hz, 2H), 4.36 (t, J=7.1 Hz, 1H), 4.23 (dd, J=8.7, 6.7 Hz, 1H), 4.07 (t, J=4.3 Hz, 4H), 3.82 (s, 63H), 3.72 (s, 2H), 3.71 (s, 19H), 3.61 (dq, J=16.6, 3.8 Hz, 5H), 3.59-3.43 (m, 12H), 3.32 (d, J=7.8 Hz, 4H), 2.98 (d, J=19.0 Hz, 1H), 2.46 (d, J=7.5 Hz, 1H), 2.43-2.35 (m, 1H), 2.13 (s, 3H), 1.87 (s, 3H), 1.62-1.54 (m, 6H), 1.23 (q, J=7.1 Hz, 9H), 0.84 (dd, J=12.5, 6.8 Hz, 6H), 0.73 (s, 3H).

### Synthesis 1C-15. LP-S13-2

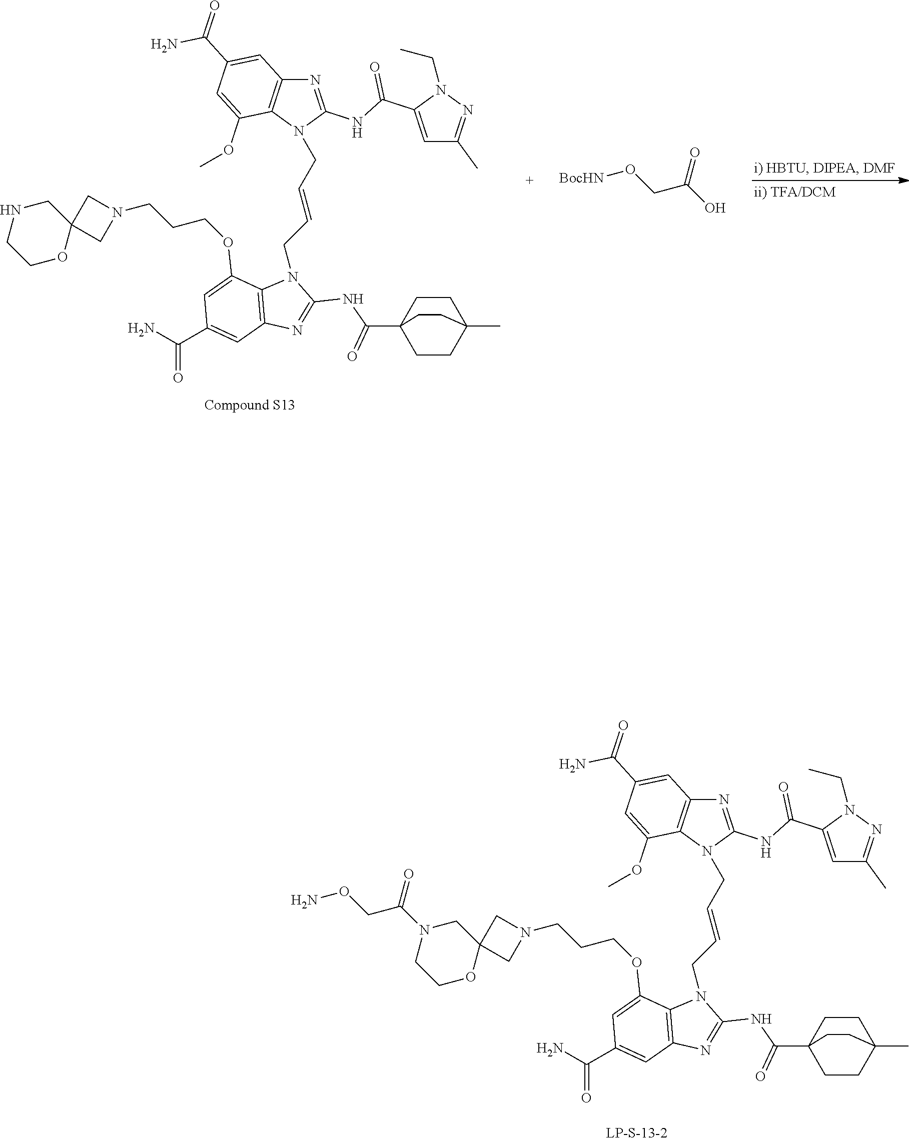 [SMILES: CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(CNCCO4)C3)c21 ; CC(C)(C)OC(=O)NOCC(=O)O ; CCn1nc(C)cc1C(=O)Nc1nc2cc(C(N)=O)cc(OC)c2n1C/C=C/Cn1c(NC(=O)C23CCC(C)(CC2)CC3)nc2cc(C(N)=O)cc(OCCCN3CC4(C3)CN(C(=O)CON)CCO4)c21]

A mixture of tert-butyl (E)-(2-(2-(3-((5-carbamoyl-1-(4-(5-carbamoyl-2-(1-ethyl-3-methyl-1H-pyrazole-5-carboxamido)-7-methoxy-1H-benzo[d]imidazol-1-yl)but-2-en-1-yl)-2-(4-methylbicyclo[2.2.2]octane-1-carboxamido)-1H-benzo[d]imidazol-7-yl)oxy)propyl)-5-oxa-2,8-diazaspiro[3.5]nonan-8-yl)-2-oxoethoxy)carbamate (Compound S13, 14.4 mg, 0.013 mmol) in 2 mL of 5% (v/v) trifluoracetic acid in 1,1,1,3,3,3-Hexafluoroisopropanol (HFIP) was stirred at room temperature for 1 hour. The reaction mixture was concentrated. The residue was diluted with DMSO (2 mL) and purified by reverse phase preparative HPLC to give LP-S13-2 (7.8 mg). Analytical data, LC-MS [ESI]: Calculated for C₄₉H₆₄N₁₃O₉[M+H⁺]: 978.49, observed 978.5. ¹H NMR (400 MHz, DMSO) δ 7.98 (s, 1H), 7.73-7.64 (m, 2H), 7.40-7.29 (m, 4H), 6.54 (s, 1H), 5.60 (s, 1H), 4.86 (d, J=5.7 Hz, 2H), 4.78 (s, 2H), 4.61 (s, 2H), 4.51 (q, J=7.1 Hz, 2H), 4.09 (s, 4H), 3.94 (s, 2H), 3.73 (d, J=5.1 Hz, 5H), 3.33 (s, 4H), 2.14 (s, 3H), 1.89 (s, 2H), 1.59 (t, J=8.0 Hz, 6H), 1.24 (q, J=7.1 Hz, 9H), 0.74 (s, 3H).

### Synthesis 1C-16. LP-S14-1

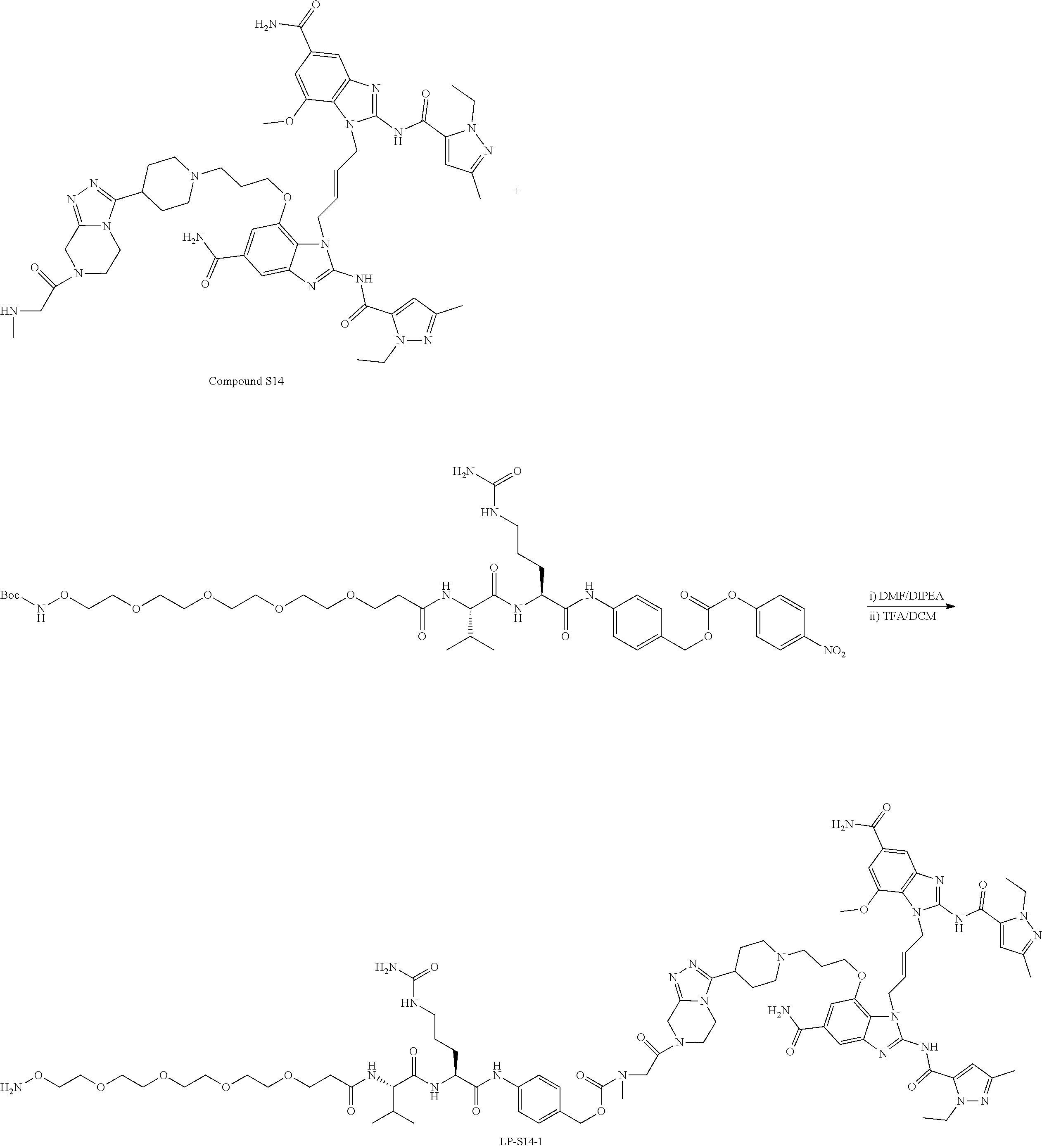 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CNC)C5)CC3)c21 ; CC(C)[C@H](NC(=O)CCOCCOCCOCCOCCONC(=O)OC(C)(C)C)C(=O)N[C@@H](CCCNC(N)=O)C(=O)Nc1ccc(COC(=O)Oc2ccc([N+](=O)[O-])cc2)cc1 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)OCc4ccc(NC(=O)[C@H](CCCNC(N)=O)NC(=O)[C@@H](NC(=O)CCOCCOCCOCCOCCON)C(C)C)cc4)C5)CC3)c21]

A mixture of 4-((23S,26S)-23-isopropyl-2,2-dimethyl-4,21,24-trioxo-26-(3-ureidopropyl)-3,6,9,12,15,18-hexaoxa-5,22,25-triazaheptacosan-27-amido)benzyl (2-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7(8H)-yl)-2-oxoethyl)(methyl)carbamate (30 mg, 0.0166 mmol) in 2 mL of 20% TFA in DCM was stirred at room temperature for 0.5 hour. The reaction mixture was concentrated. The residue was diluted with DMSO (2 mL) and purified by reverse phase preparative HPLC (acetonitrile/water+0.1% TFA) to afford 4-((17S,20S)-1-(aminooxy)-17-isopropyl-15,18-dioxo-20-(3-ureidopropyl)-3,6,9,12-tetraoxa-16,19-diazahenicosan-21-amido)benzyl (2-(3-(1-(3-((5-carbamoyl-1-((E)-4-(5-carbamoyl-2-(1-ethyl-3-methyl-1H-pyrazole-5-carboxamido)-7-methoxy-1H-benzo[d]imidazol-1-yl)but-2-en-1-yl)-2-(1-ethyl-3-methyl-1H-pyrazole-5-carboxamido)-1H-benzo[d]imidazol-7-yl)oxy)propyl)piperidin-4-yl)-5,6-dihydro-[1,2,4]triazolo[4,3-a]pyrazin-7(8H)-yl)-2-oxoethyl)(methyl)carbamate (LP-S14-1) as white solid, 21.8 mg). LCMS (ESI) m/z 1710.4 (M+H). ¹H NMR (400 MHz, DMSO) δ 12.89 (s, 2H), 11.04 (s, 1H), 10.01 (s, 1H), 8.16-8.08 (m, 2H), 8.01 (s, 1H), 7.96 (s, 1H), 7.87 (d, J=8.7 Hz, 1H), 7.67 (s, 1H), 7.60 (d, J=8.2 Hz, 1H), 7.55 (s, 1H), 7.38 (s, 3H), 7.31 (d, J=9.1 Hz, 2H), 7.24 (s, 1H), 6.97-6.89 (m, 2H), 6.53 (s, 3H), 5.99 (s, 1H), 5.77 (d, J=17.0 Hz, 2H), 5.42 (s, 2H), 5.00 (d, J=12.5 Hz, 2H), 4.92 (d, J=13.6 Hz, 3H), 4.86 (s, 1H), 4.79 (s, 1H), 4.53 (s, 4H), 4.38 (s, 1H), 4.32 (s, 1H), 4.23 (d, J=8.3 Hz, 2H), 4.00 (s, 7H), 3.94 (s, 2H), 3.72 (s, 3H), 3.65-3.58 (m, 4H), 3.50 (dd, J=8.5, 6.5 Hz, 11H), 3.10 (s, 2H), 3.01 (s, 1H), 2.95 (s, 3H), 2.86 (dd, J=18.6, 9.2 Hz, 4H), 2.12 (d, J=11.8 Hz, 5H), 2.05 (s, 2H), 1.94 (s, 4H), 1.90 (s, 1H), 1.69 (s, 1H), 1.59 (s, 1H), 1.27 (dd, J=16.3, 8.9 Hz, 7H), 0.89-0.79 (m, 6H).

### Synthesis 1C-17. LP-S14-2

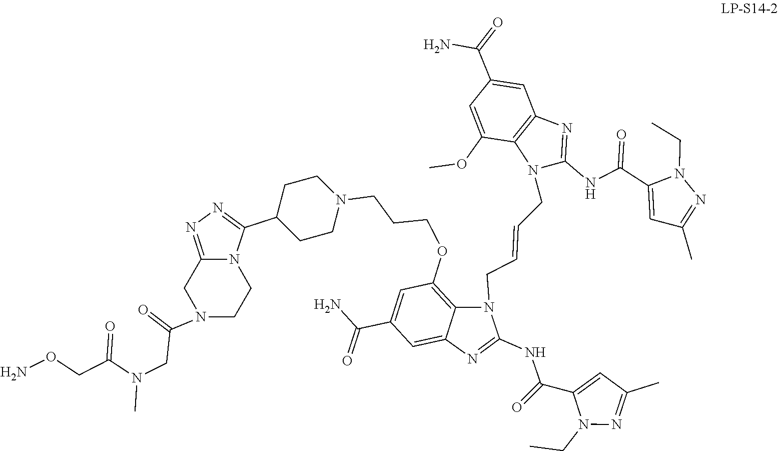 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCC(c4nnc5n4CCN(C(=O)CN(C)C(=O)CON)C5)CC3)c21]

LP-S14-2 was synthesized in an analogous fashion using the same methods as described above. Analytical data: LCMS (ESI) m/z 1114.6 (M+H). ¹H NMR (400 MHz, DMSO) δ 12.88 (s, 2H), 9.36 (s, 1H), 8.01 (s, 1H), 7.96 (s, 1H), 7.68 (s, 1H), 7.41-7.28 (m, 4H), 6.57-6.47 (m, 2H), 5.80 (s, 1H), 5.75 (s, 1H), 4.91 (d, J=14.7 Hz, 4H), 4.86-4.77 (m, 2H), 4.74 (s, 1H), 4.59-4.48 (m, 5H), 4.44 (d, J=12.3 Hz, 1H), 4.37 (s, 1H), 4.01 (s, 5H), 3.96 (s, 1H), 3.72 (d, J=8.8 Hz, 3H), 3.10 (s, 1H), 2.89 (dd, J=24.3, 10.4 Hz, 4H), 2.60 (s, 1H), 2.11 (dd, J=10.0, 5.3 Hz, 7H), 1.91 (s, 4H), 1.34-1.21 (m, 6H).

### Synthesis 1C-18. LP-S15-1

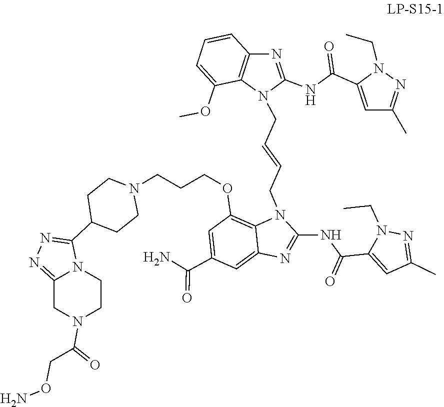 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCC(c4nnc5n4CCN(C(=O)CON)C5)CC3)c21]

Non-cleavable LP-S15-1 was synthesized and purified by reverse phase preparative HPLC using the same methods as described. Analytical data: LCMS (ESI) m/z 1000.4 (M+H). ¹H NMR (400 MHz, DMSO) δ 12.89 (s, 1H), 12.72 (s, 1H), 9.36 (s, 1H), 7.96 (s, 1H), 7.67 (s, 1H), 7.38 (s, 1H), 7.29 (d, J=1.4 Hz, 1H), 7.17-7.06 (m, 2H), 6.78 (dd, J=6.4, 2.9 Hz, 1H), 6.51 (d, J=14.9 Hz, 2H), 5.86-5.74 (m, 1H), 4.96-4.86 (m, 4H), 4.80 (s, 2H), 4.70 (s, 2H), 4.52 (q, J=7.1 Hz, 4H), 4.00 (s, 4H), 3.95 (s, 2H), 3.85 (s, 1H), 3.62 (d, J=3.5 Hz, 3H), 3.12 (s, 2H), 2.95 (s, 2H), 2.11 (dd, J=9.7, 3.7 Hz, 6H), 2.04 (s, 2H), 1.94 (s, 4H), 1.36-1.21 (m, 6H).

### Example 1D. Synthesis of Select Cytotoxin Linker-Payloads

### Synthesis 1D-1. LP-C1 (Exatecan-LP)

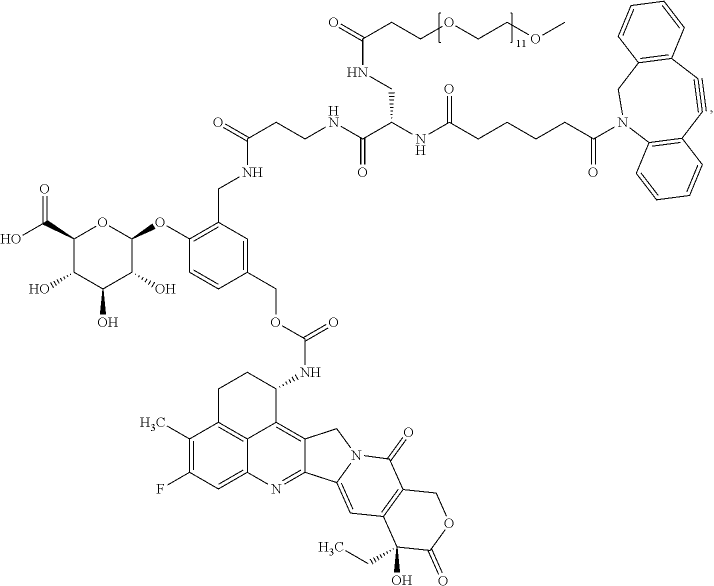

LP-C1 was synthesized according to the procedure in PCT Application WO 2024/006272, which is incorporated by reference for all purposes.

### Synthesis 1D-2. LP-C2 (Taltobulin (3-Aminophenyl Hemiasterlin)-LP)

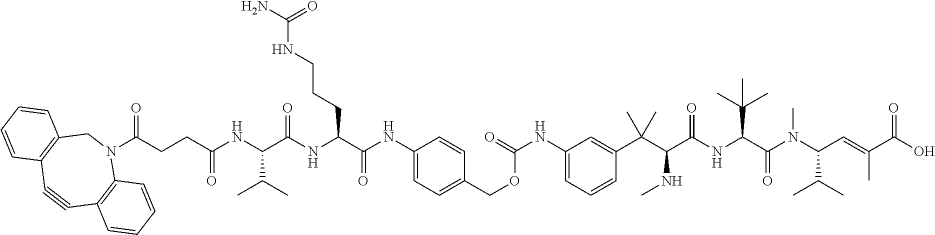 [SMILES: CN[C@H](C(=O)N[C@H](C(=O)N(C)[C@H](/C=C(\C)C(=O)O)C(C)C)C(C)(C)C)C(C)(C)c1cccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c1]

LP-C2 was synthesized according to the procedure in PCT Application WO 2016/123582, which is incorporated by reference for all purposes.

### Synthesis 1D-3. LP-C3 (Maytansine-LP)

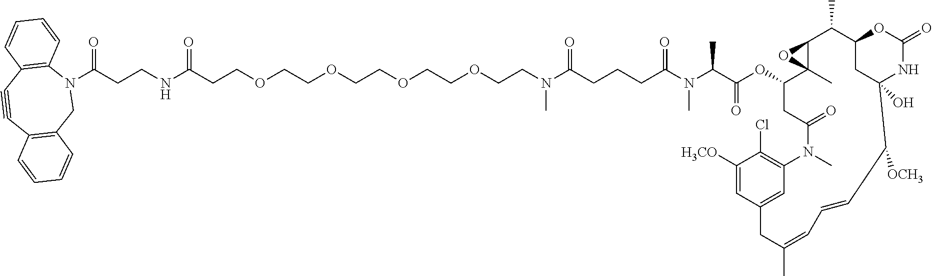 [SMILES: COc1cc2cc(c1Cl)N(C)C(=O)C[C@H](OC(=O)[C@H](C)N(C)C(=O)CCCC(=O)N(C)CCOCCOCCOCCOCCC(=O)NCCC(=O)N1Cc3ccccc3C#Cc3ccccc31)[C@]1(C)O[C@H]1[C@H](C)[C@@H]1C[C@@](O)(NC(=O)O1)[C@H](OC)/C=C/C=C(/C)C2]

LP-C3 was synthesized according to the procedure in PCT Application WO 2017/132617, which is incorporated by reference for all purposes.

### Synthesis 1D-4. LP-C4 (Exatecan-LP)

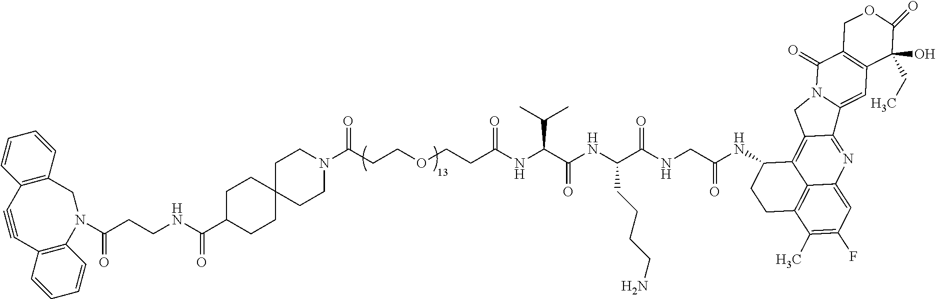

LP-C4 was synthesized according to the procedure in PCT Application WO2024/015229, which is incorporated by reference for all purposes.

### Synthesis 1D-5. LP-C5 (MMAE-LP)

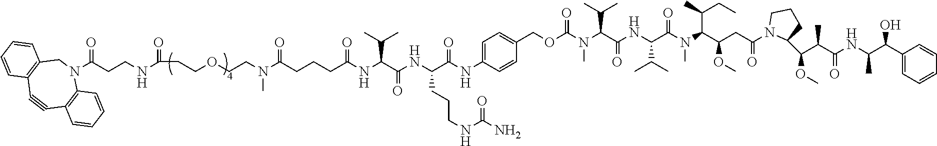

LP-C5 was synthesized according to the procedure in PCT/US2023/026769, which is incorporated by reference for all purposes.

### Example 2: Production of Anti-TROP2 Antibody

An anti-Trop2 antibody, h1925-C06, was discovered from a phage display library constructed from Rabbit immunized with TROP2 ECD and then humanized as described below. The final humanized antibody binds to human TROP2 with high affinity (Kd=4.6 nM by Biacore). Another anti-Trop2 antibody, 2293-C04, was discovered from a synthetic Ribosome display human antibody library and affinity matured as described below. The affinity matured antibody binds to human TROP2 with high affinity (Kd=1.1 nM by Biacore). Anti-Trop2 antibodies containing both pAcF and pAMF were produced using a heavy chain expressed in the cell free synthesis reaction and a prefabricated light chain (PFLC) protein. The methods below describe the rabbit antibody phage display, the humanization of the rabbit antibody, ribosome display selection, production of PFLC, and the CF synthesis of anti-Trop2 antibodies.

### Rabbit Antibody Phage Display

The initial antibody 1925-C06/1915-G02 was derived from immunizing rabbits (YZ5479 and YZ5480) with Trop-2-Fc (R&D Systems cat #650-T2) at YenZym Antibodies, LLC. Spleens were harvested and scFv antibody fragment phage libraries were constructed by standard methods (Ayyar et al Appl Micobiol Biotechnol 2015, 99, 2693-703) using a commercially available p3 phagemid vector (Antibody Design Labs). Phage libraries were selected against Trop-2-Fc protein using standard methods (Marks and Bradbury Methods Mol Biol 2004, 248, 161-76). Clonal phage ELISAs were performed against Trop-2-Fc and Fc to identify Trop-2 specific antibodies.

### Humanization of Rabbit Antibody

A phage of immune rabbit scFvs identified by phage display as described were humanized onto consensus human frameworks, and selected for humanization.

Human germline framework sequences from family IGHV3 and IGHJ4 were used for “CDR-grafting” the heavy chain variable region of the selected rabbit lead antibodies. Some mutations were designed in the frameworks of “CDR-grafted” heavy chain variable region sequences.

Human germline framework sequences from family IGKV1, IGKV3 and IGKJ4 were used for “CDR-grafting” the light chain variable region of the selected rabbit lead antibodies. Some mutations were designed in the frameworks of “CDR-grafted” light chain variable region sequences.

Humanized heavy chain variable region and light chain variable region sequences were fused to human IGHG1 and IGKC constant region sequences respectively to construct full-length humanized antibody heavy chain and light chain sequences.

### Ribosome Display Selections

Briefly, antibody Fab libraries were constructed using a standard overlap extension PCR protocol (Kechman and Pease Nat Protoc 2007, 2, 924-932) with mutagenic primers targeting complementary determining regions (CDRs). Selections for novel antibodies were performed using standard ribosome display protocols (Hanes and Pluckthun Proc Natl Acad Sci USA 1997, 94, 4937-4942). Specifically, Fab-based (Stafford et al Protein Eng Des Sel 2014, 4, 97-109). Ribosome display selections were performed according to published protocols (Dreier and Plückthun Method Mol Biol Clifton, NJ 2011, 687, 283-306) selecting against biotinylated Trop-2-Fc. After multiple rounds of selection, the DNA from RT-PCR output was cloned into an optimized vector for cell-free expression (Yin et al mAbs 2012, 4, 217-25) using standard molecular biology techniques. All constructs were HIS- and FLAG-tagged to streamline purification and testing during screening.

### Method to Produce Antibody Using a Heavy Chain Expressed in the Cell Free Synthesis Reaction and a Prefabricated Light Chain Protein

Pre-fabricated light chain protein (PFLC) is described herein and in PCT Application WO 2022/056361, which is incorporated by reference for all purposes. PFLC was expressed in E. coli using standard recombinant protein expression and purification methods commonly known in the art. A commercially available affinity resin based on protein L was used in the purification process, but other purification methods can be used.

XpressCF+™ reactions were performed with the same method whether using PFLC or plasmid-directed expression of the light chain protein. For PFLC, the LC plasmid was omitted and the PFLC stock solution was added at an optimized concentration to maximize titer and product quality. Typically about 1.0 g/L PFLC was used in XCF reaction for maximum titer. PFLC concentrations from about 0.4 to 1.5 g/L also produced good results, but the titer benefit may not be maximal at the lower PFLC concentrations. Higher concentrations of PFLC did not continue to increase titer beyond a certain point.

General XpressCF+™ procedures are known in the art (see Zawada, J. F., et al, (2011) Microscale to manufacturing scale-up of cell-free cytokine production—a new approach for shortening protein production development timelines. Biotechnol. Bioeng., 108: 1570-1578; and Groff, D., et al., (2014) Engineering toward a bacterial “endoplasmic reticulum” for the rapid expression of immunoglobulin proteins, mAbs, 6:3, 671-678. [0140] Previously described XpressCF+™ procedures were improved by the addition of a nutrient feed during the expression process to supply additional energy source, amino acids, nucleotides, and XpressRNAP. The addition of the feed increased product titers.

pAcF containing light chain production: To produce the plasmid for a light chain of an anti-Trop2 antibody, LC_aTrop2_hl925-C06_Y180/F404_K42, the product gene was synthesized with the lysine at codon K42 mutated to TAG and then cloned into pJ411 by ATUM (Redwood City, CA). An amino acid sequence of LC_aTrop2_hl925-C06_Y180/F404_K42 is shown in SEQ ID NO: 14. The vector has a kanamycin resistance marker, pUC high copy origin of replication, and the expression cassette has a T7 promoter for high level transcription. Plasmid sequences were verified by sequencing. To produce the plasmid encoding the sequences for the pAcF RS and amber suppressor tRNA, both genes were synthesized in a bicistronic operon and cloned into a medium copy pJ434 plasmid. The amber suppressor tRNA was located 5′ to the pAcF RS coding sequence and separated by a spacer sequence. This vector has a carbenicillin resistance marker and p15A medium copy origin of replication. The expression cassette has both a T7 promoter and constitutive Pc0 promoter for high level transcription before and after induction. Plasmid sequences were verified by sequencing. To make LC_aTrop2_h1925-C06_Y180/F404_K42 (SEQ ID NO: 14) expression strain SBDG782, both plasmids were transformed into E. coli SBDG756 and plated on selective media.

Protein expressions were performed in shake flasks. A single colony was picked from transformation plates into 2-3 mL of Terrific Broth (Teknova, Hollister, CA) containing appropriate antibiotic(s). After overnight incubation at 37° C. in a shaking incubator at 250 RPM, cultures were inoculated into 50 mL of fresh Terrific Broth+antibiotics (50 μg/mL kanamycin and, when applicable, 100 μg/mL carbenicillin) in a shake flask. Cultures were incubated at 37° C. in a shaking incubator at 250 RPM until the optical density at 595 nm (OD595) reached 1.0-2.0. At this time, 0.2% arabinose was added to induce expression of the protein(s) of interest and pAcF was added at 2 mM. Cultures were transferred to 25° C. for expression of LC SP11743. After expression for 16-18 hours, cells were harvested by centrifugation for 5 minutes at ˜7,000 g. Cells were resuspended in 10 mL per gram of wet cells in phosphate buffered saline (PBS) containing 0.1 mg/mL lysozyme and benzonase. After incubation on ice for 30 minutes, cells were lysed by sonication. Soluble lysates were isolated by centrifugation at >20,000 g for 30 minutes.

Cell Free Reaction to Produce pAcF and pAMF Containing Antibodies:

The CFPS reaction mix included 37.5% (v/v) S30 E. coli cell extract, 3 mg/L plasmid DNA that encodes HC of antibodies with TAG codons at either Y180 and F404 position (SEQ ID NO: 12)) or F241 and F404 position (SEQ ID NO:13), 0.5 g/L pAcF containing LC_aTrop2_h1925-C06_Y180/F404_K42, and 2 mM pAMF as well as the 20 natural amino acids, NMPs, polyamines, and small molecules for energy generation (Cai et al. Biotechnol. Prog. 2015, 31, 823-831). M. jannaschii pAMF tRNA synthetase, M. jannaschii amber suppressor tRNA, and T7 RNA polymerase were individually over-expressed in intact E. coli cells and added to the CF reaction as a crude lysate at a final concentration of 1-2% each. Reactions were conducted in a stirred tank bioreactor for 14 hrs at 25° C. at pH 7.0 and 20% DO followed by 3 hrs at pH 8.0 80% DO. mAbs were captured by Protein A affinity chromatography eluted with eluted with 100 mM Glycine at pH 3.2. The neutralized Protein A eluate was concentrated and further polished by preparative SEC (Superdex 200) in phosphate-buffered saline (1×PBS).

### Example 3: Production of iADCs

TABLE 1
Description of Linker-Payloads (LPs) used in the Examples

| | | Linker | Payload | Payload |
|---|---|---|---|---|
| LP No. | Linker name | type | name | type |
| Exatecan-LP | bGlu | Cleavable | — | Exatecan |
| (LP-C1) | sensitive | | | |
| Taltobulin | Cat-B | Cleavable | — | Taltobulin |
| (3-Amino- | cleavable | | | (a |
| phenyl | | | | hemiasterlin |
| Hemi- | | | | analog) |
| asterlin)- | | | | |
| LP (LP-C2) | | | | |
| Maytansine- | Non-cleavable | Non- | — | Maytansine |
| LP (LP-C3) | | cleavable | | |
| Exatecan-LP | Cat-B | Cleavable | — | Exatecan |
| (LP-C4) | cleavable | | | |
| | aminooxy | | | |
| MMAE-LP | Cat-B | Cleavable | — | MMAE |
| (LP-C5) | cleavable | | | |
| LP-S1-1 | Non cleavable | Non- | Compd S1 | STING agonist |
| | aminooxy | cleavable | | |
| LP-S1-2 | Cat-B | Cleavable | Compd S1 | STING agonist |
| | cleavable | | | |
| LP-S1-3 | bGlu | Cleavable | Compd S1 | STING agonist |
| | sensitive | | | |
| LP-S2-3 | Cat-B | Cleavable | Compd S2 | STING agonist |
| | cleavable | | | |
| LP-S2-1 | Non cleavable | Non- | Compd S2 | STING agonist |
| | aminooxy | cleavable | | |
| LP-S2-2 | Legumain | Cleavable | Compd S2 | STING agonist |
| | cleavable | | | |
| | aminooxy | | | |
| LP-S3-1 | Non cleavable | Non- | Compd S3 | STING agonist |
| | aminooxy | cleavable | | |
| LP-S3-2 | Non cleavable | Non- | Compd S3 | STING agonist |
| | aminooxy | cleavable | | |
| LP-S3-3 | Non cleavable | Non- | Compd S3 | STING agonist |
| | aminooxy | cleavable | | |
| LP-S3-4 | Cat-B | Cleavable | Compd S3 | STING agonist |
| | cleavable | | | |
| LP-S4-1 | Non cleavable | Non- | Compd S4 | STING agonist |
| | aminooxy | cleavable | | |
| LP-S5-1 | Non cleavable | Non- | Compd S5 | STING agonist |
| | aminooxy - | cleavable | | |
| LP-S6-1 | Legumain | Cleavable | Compd S6 | STING agonist |
| | cleavable | | | |
| | aminooxy | | | |
| LP-S7-1 | Non cleavable | Non- | Compd S7 | STING agonist |
| | aminooxy | cleavable | | |
| LP-S8-1 | Esterase | Cleavable | Compd S8 | STING agonist |
| | cleavable | | | |
| LP-S9-1 | Cat-B | Cleavable | Compd S9 | STING agonist |
| | cleavable | | | |
| LP-S3-5 | bGlu | Cleavable | Compd S3 | STING agonist |
| | sensitive | | | |
| LP-S10-1 | Non cleavable | Non- | Compd S10 | STING agonist |
| | aminooxy | cleavable | | |
| LP-S11-1 | Cat-B | Cleavable | Compd S11 | STING agonist |
| | cleavable | | | |
| LP-S12-1 | Non cleavable | Non- | Compd S12 | STING agonist |
| | aminooxy | cleavable | | |
| LP-S13-1 | Cat-B | Cleavable | Compd S13 | STING agonist |
| | cleavable | | | |
| LP-S13-2 | Non cleavable | Non- | Compd S13 | STING agonist |
| | aminooxy | cleavable | | |
| LP-S14-1 | Cat-B | Cleavable | Compd S14 | STING agonist |
| | cleavable | | | |
| LP-S14-2 | Non cleavable | Non- | Compd S14 | STING agonist |
| | aminooxy | cleavable | | |
| LP-S15-1 | Non cleavable | Non- | Compd S15 | STING agonist |
| | aminooxy | cleavable | | |

Conjugation: STING agonist linker payloads were dissolved in DMSO to a final concentration of 50 mM. Cytotoxin linker payloads were dissolved in DMD to a final concentration of 10 mM. The STING linker payload was conjugated first in 0.1 M sodium acetate, pH 4.5 buffer at concentrations between 5-50 mg/mL and with a 2-50 molar fold excess of linker payload to moles of pAcF labeled antibodies. A final amount of 3-35% DMSO was included to ensure solubility of the linker payloads. All reactions were allowed to run at temperatures ranging between 22-37° C., for 4 to 96 hours. After STING agonist linker payload conjugation reached target drug to protein ratio, pH of the conjugation reaction was adjusted to pH 7, and then the cytotoxin linker payload was added at drug to pAMF molar ratio of 3-10. The conjugation reaction continued at temperatures ranging between 22-37° C. for 4-24 hours. LCMS was used to measure DAR. Table 2 shows the DALR values of ADCs and iADCs.

TABLE 2
DAR value of ADCs and iADCs

| | | | STING | | | |
|---|---|---|---|---|---|---|
| | | toxin | agonist | | STING | |
| ADC or | | conjugation | conjugation | | agonist | |
| iADC # | mAb | sites | site | Toxin LP | LP | DAR |
| ADC 1 | aTrop2_2293-C04- | Y180/F404 | K42 | LP-C1 | — | 3.9 |
| | Hc_W103A | | | | | |
| ADC 2 | aTrop2_2293-C04- | Y180/F404 | K42 | LP-C1 | — | 3.9 |
| | Hc_W50A, W103A | | | | | |
| ADC 3 | aTrop2_h1925-C06 | Y180/F404 | K42 | LP-C1 | — | — |
| ADC 4 | aTrop2_h1925-C06 | Y180/F404 | K42 | LP-C1 | | 3.9 |
| ADC 5 | aTrop2_h1925-C06 | F241/F404 | K42 | LP-C1 | | 3.9 |
| ADC 6 | aTrop2_h1925-C06 | F241/F404 | K42 | LP-C1 | — | 3.85 |
| iADC 1 | aTrop2_h1925-C06 | Y180/F404 | K42 | LP-C1 | LP-S1-1 | 5.7 |
| iADC 2 | aTrop2_h1925-C06 | Y180/F404 | K42 | LP-C1 | LP-S1-2 | 5.7 |
| iADC 3 | aTrop2_h1925-C06 | Y180/F404 | K42 | LP-C1 | LP-S1-3 | 5.4 |
| iADC 4 | aTrop2_h1925-C06 | Y180/F404 | K42 | LP-C1 | LP-S4-1 | 5.6 |
| iADC 5 | aTrop2_h1925-C06 | Y180/F404 | K42 | LP-C1 | LP-S2-2 | 5.6 |
| iADC 6 | aTrop2_h1925-C06 | Y180/F404 | K42 | LP-C1 | LP-S6-1 | 5.67 |
| iADC 7 | aTrop2_h1925-C06 | Y180/F404 | K42 | LP-C1 | LP-S8-1 | 5.9 |
| iADC 8 | aTrop2_h1925-C06 | Y180/F404 | K42 | LP-C1 | LP-S2-1 | 6 |
| iADC 9 | aTrop2_h1925-C06 | F241/F404 | K42 | LP-C1 | LP-S1-1 | 5.1 |
| iADC 10 | aTrop2_h1925-C06 | Y180/F404 | K42 | LP-C1 | LP-S5-1 | 5.9 |
| iADC 11 | aTrop2_h1925-C06 | F241/F404 | K42 | LP-C1 | LP-S4-1 | 5.2 |
| iADC 12 | aTrop2_h1925-C06 | Y180/F404 | K42 | LP-C2 | LP-S4-1 | 5.76 |
| iADC 13 | aTrop2_h1925-C06 | Y180/F404 | K42 | LP-C1 | LP-S7-1 | 5.6 |
| iADC 14 | aTrop2_h1925-C06 | Y180/F404 | K42 | LP-C1 | LP-S9-1 | 5.5 |
| iADC 15 | aTrop2_h1925-C06 | Y180/F404 | K42 | LP-C1 | LP-S3-1 | 5.57 |
| iADC 16 | aTrop2_h1925-C06 | Y180/F404 | K42 | LP-C1 | LP-S3-2 | 5.83 |
| iADC 17 | aTrop2_h1925-C06 | Y180/F404 | K42 | LP-C1 | LP-S3-3 | 5.84 |
| iADC 18 | aTrop2_h1925-C06 | Y180/F404 | K42 | LP-C1 | LP-S3-4 | 5.78 |
| iADC 19 | aTrop2_h1925-C06 | Y180/F404 | K42 | LP-C1 | LP-S3-5 | 5.35 |
| iADC 20 | aTrop2_h1925-C06 | Y180/F404 | K42 | LP-C1 | LP-S10-1 | 4.14 |
| iADC 21 | aTrop2_h1925-C06 | F241/F404 | K42 | LP-C1 | LP-S3-3 | 5.54 |
| iADC 22 | aTrop2_h1925-C06 | Y180/F404 | K42 | LP-C1 | LP-S2-3 | 5.94 |
| iADC 23 | aTrop2_h1925-C06 | Y180/F404 | K42 | LP-C2 | LP-S1-1 | 5.9 |
| iADC 24 | aTrop2_h1925-C06 | Y180/F404 | K42 | LP-C4 | LP-S1-1 | 5.64 |
| iADC 25 | aTrop2_h1925-C06 | Y180/F404 | K42 | LP-C5 | LP-S1-1 | 5.87 |
| iADC 26 | aTrop2_h1925-C06 | Y180/F404 | K42 | LP-C3 | LP-S1-1 | 5.82 |
| iADC 27 | aTrop2_h1925-C06 | Y180/F404 | K42 | LP-C4 | LP-S1-2 | 5.91 |
| iADC 28 | aTrop2_h1925-C06 | Y180/F404 | K42 | LP-C5 | LP-S1-2 | 5.76 |
| iADC 29 | aTrop2_h1925-C06 | Y180/F404 | K42 | LP-C3 | LP-S1-2 | 5.91 |
| iADC 30 | aTrop2_h1925-C06 | Y180/F404 | K42 | LP-C1 | LP-S1-3 | 5.94 |
| iADC 31 | aTrop2_h1925-C06 | Y180/F404 | K42 | LP-C5 | LP-S1-3 | 5.94 |
| iADC 32 | aTrop2_h1925-C06 | Y180/F404 | K42 | LP-C3 | LP-S1-3 | 5.81 |
| iADC 33 | aTrop2_h1925-C06 | Y180/F404 | K42 | LP-C1 | LP-S3-1 | 5.93 |
| iADC 34 | aTrop2_h1925-C06 | Y180/F404 | K42 | LP-C5 | LP-S3-1 | 5.78 |
| iADC 35 | aTrop2_h1925-C06 | Y180/F404 | K42 | LP-C3 | LP-S3-1 | 4.55 |
| iADC 36 | aTrop2_h1925-C06 | Y180/F404 | K42 | LP-C1 | LP-S11-1 | 5.9 |
| iADC 37 | aTrop2_h1925-C06 | Y180/F404 | K42 | LP-C1 | LP-S12-1 | 5.9 |
| iADC 38 | aTrop2_h1925-C06 | Y180/F404 | K42 | LP-C1 | LP-S13-1 | 5.9 |
| iADC 39 | aTrop2_h1925-C06 | Y180/F404 | K42 | LP-C1 | LP-S13-2 | 5.9 |
| iADC 40 | aTrop2_h1925-C06 | Y180/F404 | K42 | LP-C1 | LP-S14-1 | 5.9 |
| iADC 41 | aTrop2_h1925-C06 | Y180/F404 | K42 | LP-C1 | LP-S-14-2 | 5.9 |
| iADC 42 | aTrop2_h1925-C06 | Y180/F404 | K42 | LP-C1 | LP-S15-1 | 5.9 |
| iADC 43 | aTrop2_h1925-C06 | Y180/F404 | K42 | LP-C2 | LP-S1-1 | 5.8 |
| iADC 44 | aTrop2_h1925-C06 | Y180/F404 | K42 | LP-C1 | LP-S3-4 | 5.9 |

### Example 3. ADC and iADC Thermostability

A protein thermal shift assay was carried out by mixing the protein to be assayed with an environmentally sensitive dye (SYPRO Orange, Life Technologies Cat #S-6650) in a phosphate buffered solution (PBS), and monitoring the fluorescence of the mixture in real time as it underwent controlled thermal denaturation. Protein solutions between 0.2-2 mg/mL were mixed at a 1-1 volumetric ratio with a 1-250 PBS-diluted solution of SYPRO Orange (SYPRO Orange stock dye is 5000× in DMSO). 6 μL aliquots of the protein-dye mixture were dispensed in quadruplicate in a 384-well microplate (Bio-Rad Cat #MSP-3852), and the plate was sealed with an optically clear sealing film (Bio-Rad Cat #MSB-1001) and placed in a 384-well plate real-time thermocycler (Bio-Rad CFX384 Real Time System). The protein-dye mixture was heated from 25° C. to 95° C., at increments of 0.2° C. per cycle (˜1.5° C. per minute), allowing 3 seconds of equilibration at each temperature before taking a fluorescence measurement. At the end of the experiment, the transition melting temperatures (TM1 and TM2) were determined using the Bio-Rad CFX manager software. TM1 represents the melting temperature of the Fc domain. TM2 represents the melting temperature of the Fab domain.

For the colloidal stability of an antibody, the average particle radius (smaller radius=more tightly folded) and polydispersity (range of measured radii−an indicator of homogeneity) were measured in 1×PBS+10% sucrose buffer. While the radius and polydispersity did increase after conjugation, both measurements were within acceptable parameters (radius of an antibody is typically ˜5-6 nm, while polydispersity <15% is considered a homogeneous product) and indicate that—aTrop2_h1925-C06_Y180/F404_K42 antibody is a well-behaved antibody in this buffer. Thermostability and colloidal stability of selected ADC and iADCs are shown in Table 3.

TABLE 3
ADC and iADC Thermostability and Colloidal Stability

| | | | Colloidal stability | |
|---|---|---|---|---|
| ADC or | Thermostability | | Radius, | |
| iADC | TM1, ° C. | TM2, ° C. | nm | Polydispersity, % |
| ADC 3 | 58.9 | 76.2 | 5.4 | 13.4 |
| ADC 4 | 60.8 | 75.8 | 5.8 | 19.1 |
| ADC 5 | 57.5 | 76.3 | 5.3 | 12.5 |
| ADC 6 | 66.9 | 76.2 | 5.7 | 16.4 |
| iADC 4 | 60.9 | 76.1 | 6.7 | 20.0 |
| iADC 11 | 65.9 | 73.5 | 5.5 | 11.8 |

### Example 4. IADCs Cell Binding Activity in FACS Based Binding Assay

CHO-K cells were purchased from ATCC (American Type Culture Collection) and DMEM/F-12 (50:50) high glucose medium (Corning) supplemented with 10% heat-inactivated fetal bovine serum (Corning), 1% Penicillin/Streptomycin (Corning) and 2 mM/L-glutamax (Thermo Fisher). CHO cells over-expressing human TROP2 (CHO-hTROP2) or over-expressing cynomolgus monkey TROP2 (CHO-cTROP2) were generated by transfecting CHO cells with plasmids containing full length human or monkey TROP2 sequences (SEQ ID NO: 1) to stably express human TROP2 or Cynomolgus TROP2 (SEQ ID NO:2) on the cell surface and maintained in the same cell culture medium supplied with 30 ug/mL puromycin.

200,000 cells in 50 uL FACS buffer were seeded in 96 well polypropylene plates and mixed with 50 μL of test antibodies serially diluted in FACS buffer (lx PBS with 1% BSA and 0.05% sodium azide). After incubation on ice for 60 minutes, cells were washed with FACS buffer and incubated on ice for 60 minutes with 100 uL FACS buffer containing 2.5 ug/mL R-Phycoerythrin-conjugated goat anti-Human IgG (Jackson ImmunoResearch Laboratories). Cells were washed twice, fixed in 2% paraformaldehyde in PBS (Santa Cruz Biotechnology) for 10 minutes on ice in the dark and analyzed using the Attune Flow Cytometer (Thermo Fisher). Mean fluorescence intensities were analyzed using FlowJo® software (Becton, Dickinson & Company). Binding constants were calculated using the statistical software, GraphPad Prism (GraphPad Software; La Jolla, CA) using the nonlinear regression equation, one site-specific binding with Hill slope. Secondary antibody alone was used as a control.

As shown in Table 4, ADC 3 and ADC 5 showed similar high affinity binding to CHO-hTROP2 and CHO-cTROP2, which indicated that the pAMF insertion at different sites did not change the binding affinity of the antibodies. iADC 1, iADC 9 and iADC 17 showed similar high affinity binding to human and cyno TROP2 expressed on CHO cells as the un-conjugated antibodies, which indicated that conjugation of LP-C1 and the STING LPs did not change the binding affinity of the TROP2 antibodies.

TABLE 4
TROP2 iADCs cell binding activity on CHO-hTROP2 and CHO-cTROP2 cells

| | | Parental CHO | | CHO-hTROP2 | | CHO-cTROP2 | |
|---|---|---|---|---|---|---|---|
| ADC or | | Bₘₐₓ | K_{d} | Bₘₐₓ | K_{d} | Bₘₐₓ | K_{d} |
| iADC | Description | (MFI) | (nM) | (MFI) | (nM) | (MFI) | (nM) |
| ADC 3 | h1925-C06 Y180/F404/K42 | NB | NB | 2,070 | 1.5 | 28,082 | 8.0 |
| ADC 5 | h1925-C06 F241/F404/K42 | NB | NB | 2,304 | 1.5 | 25,338 | 5.7 |
| iADC 1 | h1925-C06 Y180/F404-LP- | NB | NB | 2,355 | 2.3 | 22,004 | 2.7 |
| | C1/K42-LP-S1-1 | | | | | | |
| iADC 9 | h1925-C06 F241/F404-LP- | NB | NB | 2,340 | 3.0 | 26,789 | 5.1 |
| | C1/K42-LP-S1-1 | | | | | | |
| iADC 17 | h1925-C06 Y180/F404-LP- | NB | NB | 2,331 | 3.3 | 28,147 | 7.5 |
| | C1/K42-LP-S3-3 | | | | | | |

NB = No Binding

### Example 5. ADC, iSAC and iADC Induced Cell Killing of Cancer Cells

The cytotoxicity of the ADCs, iSACs and iADCs was measured in a cell proliferation assay. TROP2 positive NCI-H292, NCI-H441 cells and TROP2 negative NCI-H460 and NCI-H1703 cells were obtained from ATCC (American Type Culture Collection) and maintained in DMEM/F12 (1:1), high glucose (Corning) supplemented with 10% heat-inactivated fetal bovine serum (Thermo Scientific), 2 mM glutamax (Thermo Scientific), and 1× Penicillin/Streptomycin (Corning).

Cells were collected and counted by the Vi-CELL Cell Viability Analyzers (Beckman Coulter). A total of 7500 cells in a volume of 50 uL were seeded in each well of a 96-well flat bottom white Polystyrene plate. Test articles were diluted at 2× final concentration in the cell culture medium and 50 uL was added into each treatment well. Assay plates were cultured at 37° C. in a CO₂ incubator for 120 hours. For cell viability measurement, 60 μL of Cell Titer-Glo® reagent (Promega Corp. Madison, WI) was added into each well, and plates were processed as per product instructions. Relative luminescence was measured on an ENVISION® plate reader (Perkin-Elmer; Waltham, MA). Relative luminescence readings were converted to % viability using untreated cells as controls. Data was fitted with non-linear regression analysis, using log(inhibitor) vs. response-variable slope, 4 parameter fit equation using GraphPad Prism (GraphPad v 5.00, Software; San Diego, CA).

As free drugs, only exatecan exhibited good cell killing on all TROP2 positive and negative cell lines tested, while STING agonist free drugs, Compound S-1 and Compound S-3 did not result in significant cell killing. Treatment with ADC 4 resulted in more potent cell killing (IC₅₀<nM) for TROP2 positive cell lines NCI-H1292 and NCI-H441 than free exatecan. No cell killing was observed on TROP2 negative cell lines NCI-H460 and NCI-H1703 for ADC 4. iADC 1, iADC 9 and iADC 17 displayed similar cell killing activity to ADC 4 on TROP2 positive cell line. ADC and iADCs did not induce any cell killing on TROP2 negative cell lines NCI-1H460 and NCI-1H1703. iSAC 1, iSAC 2 and iSAC 14 did not result in significant cell killing for any of the cell lines tested (Table 5).

TABLE 5
Cell killing activity of free agonists, TROP2 ADC, iSAC and iADCs on Cancer Cells

| | | NCI-H292 | | NCI-H441 | | NCI-H460 | | NCI-H1703 | |
|---|---|---|---|---|---|---|---|---|---|
| | | (Trop2+) | | (Trop2+) | | (Trop2−) | | (Trop2−) | |
| | | EC₅₀ | Span | EC₅₀ | Span | EC₅₀ | Span | EC₅₀ | Span |
| Sample | Description | (nM) | (%) | (nM) | (%) | (nM) | (%) | (nM) | (%) |
| | Exatecan | 0.88 | 80 | 0.57 | 89 | 0.97 | 83 | 1.04 | 95 |
| Compound S-1 | STING agonist | >100 | <15 | >100 | <15 | >100 | <15 | >100 | <15 |
| Compound S-3 | STING agonist | >100 | <15 | >100 | <15 | >100 | <15 | >100 | <15 |
| ADC 4 | h1925-C06 | 0.39 | 81 | 0.15 | 79 | >100 | <15 | >100 | <15 |
| | Y180/F404-LP-C1/K42 | | | | | | | | |
| iSAC 1 | h1925-C06 | >100 | <15 | >100 | <15 | >100 | <15 | >100 | <15 |
| | Y180/F404/K42-LP-S1-1 | | | | | | | | |
| iADC 1 | h1925-C06 | 0.4 | 93 | 0.14 | 75 | >100 | <15 | >100 | <15 |
| | Y180/F404-LP-C1/K42-LP-S1-1 | | | | | | | | |
| iSAC 2 | h1925-C06 | >100 | <15 | >100 | <15 | >100 | <15 | >100 | <15 |
| | Y180/F404/K42-LP-S2-1 | | | | | | | | |
| iADC 9 | h1925-C06 | 0.52 | 93 | 0.16 | 77 | >100 | <15 | >100 | <15 |
| | F241/F404-LP-C1/K42-LP-S1-1 | | | | | | | | |
| iSAC 14 | h1925-C06 | >100 | <15 | >100 | <15 | >100 | <15 | >100 | <15 |
| | Y180/F404/K42-LP-S3-3 | | | | | | | | |
| iADC 17 | h1925-C06 | 0.47 | 92 | 0.21 | 78 | >100 | <15 | >100 | <15 |
| | Y180/F404-LP-C1/K42-LP-S3-3 | | | | | | | | |

### Example 6: In Vitro Human Cancer Cell and PBMCs Co-Culture Assays

The ability of different TROP2 iADCs to induce cancer cell killing, immune activation, and cytokine release was evaluated in an in vitro co-culture assay with human cancer cells and human PBMCs.

Human PBMCs were isolated from healthy human blood donors (StemCells) by Leukosep tube (Greiner Bio-One) and Nycoprep 1.077 buffer (Progen) according to manufacturer's instructions. Residual RBCs were removed using ACK lysis buffer (Gibco) for 10 minutes, followed by DPBS wash. Isolated PBMCs were counted, resuspended in freezing medium (Sigma) and stored in a freezing vessel at −80° C. for 24 hours before being transferred to vapor-phase liquid nitrogen for long-term storage.

For the co-culture assay, cancer cells were plated on transparent flat bottom polystyrene plates and allowed to attach overnight. The next day, serially diluted (1:4) test articles were formulated at 3× starting concentration in complete RPMI media and added onto each well. Human PBMCs were thawed and resuspended in RPMI supplemented with 10% heat-inactivated fetal bovine serum from Hyclone, 1% Penicillin/Streptomycin and 2 mM/L-glutamax. PBMCs were then added into the same well at a final ratio of PBMCs:cancer cells=30:1. Plates were incubated for 2 or 3 days at 37° C. with 5% CO₂ in a cell culture incubator.

On the day of sample collection, cell culture supernatants were collected and stored at −80° C. until cytokine release analyses were performed. All the cells in the well were combined (both suspension and adherent cells) and resuspended in 50 μL of PBS containing Live/Dead Fixable Violet Dead Cell Stain (Thermo Fisher) and 1× human Fc block (BD Biosciences) and incubated at 37° C. for 20 minutes. After washing with complete RPMI-1640 media and FACS buffer, cells were resuspended in 25 uL per well of a primary antibody concoction in FACS binding buffer containing 1:20 dilution of BD Horizon Brilliant Stain Buffer Plus, live/Dead staining and a series of antibodies for gating different immune cells and activation markers. After incubation on ice for 1 hour, cells were washed with FACS buffer and fixed with 50 uL/well of 2% PFA (paraformaldehyde) for 20 minutes. Cells were then washed with FACS buffer again and read on Cytek flow cytometer. FACS data analysis was done by Flowjo software to identify monocytes, B cells, CD4+ T cells, CD8+ T cells, NK cells and Dendritic cells and their activation status. Data from Flowjo was exported and plotted using non-linear regression analysis, log(agonist/inhibitor) vs. response, variable slope, 4-parameter fit equation in GraphPad Prism.

To measure the cytokine released in this assay, customized MSD V-plex plates were ordered to measure human IL6, IP-10, TNFα, IFNγ, IL-1α, MCP-1, MIP1α. The supernatant was thawed and diluted 1:50 using dilution buffer provided by the vendor before being added into the MSD plates. Cytokine measurement in the cell culture supernatant was carried out using protocols provided by the vendor. Raw data generated by the Meso Scale Discovery (MSD®) SQ120 plate reader were imported into MSD Discovery Workbench software. Unknown sample values were interpolated from standard curves by the software and imported into GraphPad Prism. Each cytokine level was fitted with non-linear regression analysis, using log(agonist) vs. response, variable slope, 4-parameter fit equation in GraphPad Prism.

### Example 7: The Evaluation of TROP2 iADCs Conjugated to STING Agonists with Different Potencies and Different Linkers in Co-Culture Assays

The ability of different TROP2 iADCs to induce cancer cell killing, immune activation, and cytokine release was evaluated in an in vitro co-culture assay with TROP2 positive CaoV3 cells or NCI-H292 cells and human PBMCs. h1925-C06, an anti-TROP2 antibody, was conjugated to LP-C1 on the heavy chain Y180/F404 sites and to different STING agonist linker payloads at light chain K42 sites.

As shown in Table 6, all the iADCs induced similar or a bit more potent killing of TROP2 positive CAOV3 cells compared to the ADC control ADC 4.

When co-cultured with TROP2 positive CAOV3 cells, ADC 4 did not change monocyte or DCs percentage in PBMCs, but induced weak monocytes and DCs activation (Table 7). All the iADCs induced a decrease of monocyte percentage in PBMCs, which is correlated with monocyte activation and an increase of DC percentage in PBMCs and DC cell activation. This data indicates that the iADCs induced monocyte activation and differentiation into DCs in this assay. iADCs also induced activation of other immune cells (B cells, CD4+ T cells, CD8+ T cells, dendritic cells as well as NK cells) with similar trends (Table 8).

As shown in Table 7 and Table 8, in general, iADCs conjugated to a non-cleavable linker (iADC 1 and iADC 4) induced more potent immune cell activation than iADCs conjugated to a cleavable linker (iADC 2, iADC 3, iADC 5). For example, iADC 1 (STING agonist Compound S-1 with a non-cleavable linker) induced more potent immune cell activation than iADC 2 (Compound S-1 with a cathepsin-B sensitive cleavable linker) and iADC 3 (Compound S-1 with a beta-glucoronadase sensitive cleavable linker). iADC 4 (STING agonist Compound S-4 with non-cleavable linker) induced similar high potency immune cell activation as iADC 1 (STING agonist Compound S-1 with non-cleavable linker). iADC conjugated to LP-S2-2 (STING agonist Compound S-2 with a legumin sensitive cleavable linker) induced similar immune cell activation as iADCs conjugated to Compound S-1 with cleavable linkers.

Activated immune cells release several cytokines to further promote immune cell recruitment and proliferation. While the TROP2 ADC induced only weak release of IP-10 and IL-6 in the co-culture system, TROP2 iADCs induced potent cytokine release (both IP-10 and IL-6) in the human tumor cells and PBMCs co-culture assay (Table 9) with trends similar to immune cell activation.

TABLE 6
TROP2 iADCs induced tumor cell killing

| | | CAOV3 Cell Count | |
|---|---|---|---|
| ADC or | | IC₅₀ | Span |
| iADC # | Description | (nM) | (Cell Count) |
| ADC 4 | h1925-C06 Y180/F404-LP- | 0.136 | 20,829 |
| | C1/K42 | | |
| iADC 1 | h1925-C06 Y180/F404-LP- | 0.070 | 17,492 |
| | C1/K42-LP-S1-1 | | |
| iADC 2 | h1925-C06 Y180/F404-LP- | 0.064 | 17,479 |
| | C1/K42- LP-S1-2 | | |
| iADC 3 | h1925-C06 Y180/F404-LP- | 0.070 | 18,501 |
| | C1/K42- LP-S1-3 | | |
| iADC 4 | h1925-C06 Y180/ LP-C1/K42-LP- | 0.069 | 20,646 |
| | S4-1 | | |
| iADC 5 | h1925-C06 Y180/ LP-C1/K42-LP- | 0.095 | 20,926 |
| | S2-2 | | |

TABLE 7
TROP2 iADCs induced immune cells activation

| | CAOV3/PBMC co-culture | | | | | | | |
|---|---|---|---|---|---|---|---|---|
| | Monocyte | | Monocyte | | | | | |
| | Cell Count | | Activation | | DC Cell Count | | DC Activation | |
| ADC or | EC₅₀ | Span | EC₅₀ | Span | EC₅₀ | Span | EC₅₀ | Span |
| iADC # | (nM) | (%) | (nM) | (%) | (nM) | (%) | (nM) | (%) |
| ADC 4 | 1.460 | 5 | 0.296 | 37 | NC | <2 | 0.24 | 9 |
| iADC 1 | 0.065 | 18 | 0.03 | 44 | 0.078 | 9 | 0.14 | 44 |
| iADC 2 | 0.098 | 18 | 0.04 | 51 | 0.131 | 9 | 0.18 | 57 |
| iADC 3 | 0.205 | 15 | 0.11 | 57 | 0.228 | 8 | 0.21 | 40 |
| iADC 4 | 0.043 | 22 | 0.03 | 37 | 0.037 | 13 | 0.19 | 52 |
| iADC 5 | 0.376 | 16 | 0.08 | 58 | 1.143 | 9 | 2.20 | 87 |

NC = Not Calculable due to incomplete curve

TABLE 8
TROP2 iADCs induced immune cells activation

| | CAOV3/PBMC co-culture | | | | | | | |
|---|---|---|---|---|---|---|---|---|
| | | | CD4+ T cell | | CD8+ T cell | | NK Cell | |
| | B cell Activation | | Activation | | Activation | | Activation | |
| ADC or | EC₅₀ | Span | EC₅₀ | Span | EC₅₀ | Span | EC₅₀ | Span |
| iADC # | (nM) | (%) | (nM) | (%) | (nM) | (%) | (nM) | (%) |
| ADC 4 | 4.583 | 19 | NC | <2 | 2.764 | 8 | 7.462 | 16 |
| iADC 1 | 0.14 | 15 | 0.291 | 65 | 0.078 | 8 | 0.110 | 14 |
| iADC 2 | 0.12 | 30 | 0.388 | 41 | 0.033 | 10 | 0.054 | 15 |
| iADC 3 | 0.26 | 14 | 0.319 | 26 | 0.160 | 11 | 0.175 | 16 |
| iADC 4 | 0.47 | 34 | 0.304 | 63 | 0.033 | 6 | 0.066 | 12 |
| iADC 5 | ~0.3 | ~30 | 1.917 | 40 | 0.090 | 10 | 0.117 | 17 |

TABLE 9
TROP2 iADCs induced cytokine release

| ADC or | CAOV3/PBMC co-culture | | | |
|---|---|---|---|---|
| iADC | IP-10 release | | IL-6 release | |
| # | EC50 (nM) | Span (pg/mL) | EC50 (nM) | Span (pg/mL) |
| ADC 4 | ~24 | ~920 | NC | <200 |
| iADC 1 | 0.12 | 923 | 0.019 | 850 |
| iADC 2 | 0.08 | 929 | 0.060 | 830 |
| iADC 3 | 0.25 | 923 | 0.096 | 959 |
| iADC 4 | 0.08 | 961 | 0.005 | 1,769 |
| iADC 5 | 0.21 | 926 | 0.118 | 1,087 |

As shown in Table 10, iADC 10 and iSAC 10, conjugated to LP-S5-1, induced slightly more potent immune cell activation than iADCs and iSACs conjugated to LP-S1-1, LP-S1-3 and LP-S2-1. iADC and iSAC conjugated to LP-S2-1 showed slightly weaker activity compared to the others. In this experiment, iSACs induced potent target cell killing with similar or better potency than the ADC control ADC 4 as well. iSAC also induced monocyte cell count decrease and immune cell activation similar to their corresponding iADCs (only monocyte activation is shown here, similar trend observed for DC, B cell, T cell and NK cell activation).

TABLE 10
TROP2 ADC, iSACs and iADCs induced tumor
cell killing and immune cell activation

| | | CAOV3/PBMC co-culture | | | | | |
|---|---|---|---|---|---|---|---|
| | | Cancer | | | | | |
| | | Cell Count | | Monocyte | | Monocyte | |
| ADC, | | | Span | Cell Count | | Activation | |
| iADC, or | | IC₅₀ | (Cell | IC₅₀ | Span | EC₅₀ | Span |
| iSAC | Description | (nM) | Count) | (nM) | (%) | (nM) | (%) |
| ADC 4 | h1925-C06 | 0.43 | 4,100 | NC | <5 | NC | <5 |
| | Y180/F404-LP-C1/K42 | | | | | | |
| iSAC 1 | h1925-C06 | 0.27 | 4,374 | 0.24 | 18 | 0.27 | 67 |
| | Y180/F404/K42-LP-S1-1 | | | | | | |
| iADC 1 | h1925-C06 | 0.26 | 3,794 | 0.22 | 18 | 0.24 | 54 |
| | Y180/F404-LP-C1/K42-LP-S1-1 | | | | | | |
| iSAC 4 | h1925-C06 | 0.11 | 5,963 | 0.27 | 19 | 0.28 | 72 |
| | Y180/F404/K42-LP-S1-3 | | | | | | |
| iADC 3 | h1925-C06 | 0.11 | 5,401 | 0.17 | 20 | 0.22 | 56 |
| | Y180/F404-LP-C1/K42-LP-S1-3 | | | | | | |
| iSAC 9 | h1925-C06 | 0.19 | 5,539 | 0.35 | 21 | 0.36 | 66 |
| | Y180/F404/K42-LP-S2-1 | | | | | | |
| iADC 8 | h1925-C06 | 0.29 | 4,861 | 0.47 | 20 | 0.56 | 54 |
| | Y180/F404-LP-C1/K42-LP-S2-1 | | | | | | |
| iSAC 10 | h1925-C06 | 0.07 | 4,791 | 0.11 | 21 | 0.08 | 64 |
| | Y180/F404/K42-LP-S5-1 | | | | | | |
| iADC 10 | h1925-C06 | 0.10 | 3,922 | 0.12 | 20 | 0.13 | 65 |
| | Y180/F404-LP-C1/K42-LP-S5-1 | | | | | | |

NC = Not Calculable due to incomplete curve

To further investigate the linker property and potency of the TROP2 iADCs, TROP2 iADCs and iSACs conjugated to STING agonist Compound S-3 with different length were further evaluated in another vitro co-culture assay with TROP2 positive CaoV3 cells and human PBMCs. In this assay (Table 11), TROP2 iADCs induced more potent cell killing and immune cell activation in this experiment compared to ADC 4. iADC 1 induced the most potent cell killing and immune cell activation among the iADCs tested. TROP2 iADC conjugated to Compound S-3 with longer linker (LP-S3-3) induced much reduced target cell killing and immune cell activation compared to other iADCs (only monocyte activation is shown here, similar trend observed for DC, B cell, T cell and NK cell activation).

TABLE 11
TROP2 ADC, iSACs and iADCs induced tumor
cell killing and immune cell activation

| | | CAOV3/PBMC co-culture | | | | | |
|---|---|---|---|---|---|---|---|
| | | Cancer | | | | | |
| | | Cell Count | | Monocyte | | Monocyte | |
| ADC, | | | Span | Cell Count | | Activation | |
| iADC, or | | IC₅₀ | (Cell | IC₅₀ | Span | EC₅₀ | Span |
| iSAC | Description | (nM) | Count) | (nM) | (%) | (nM) | (%) |
| ADC 4 | h1925-C06 | 6.19 | 13,846 | NC | <5 | 0.019 | 15 |
| | Y180/F404-LP-C1/K42 | | | | | | |
| iSAC1 | h1925-C06 | 0.14 | 14,926 | 0.21 | 21.7 | 0.007 | 24 |
| | Y180/F404 /K42-LP-S1-1 | | | | | | |
| iADC 1 | h1925-C06 | 0.05 | 15,462 | 0.09 | 20.6 | 0.01 | 27 |
| | Y180/F404-LP-C1/K42-LP-S1-1 | | | | | | |
| iSAC 11 | h1925-C06 | NC | <5000 | NC | <5 | 1.207 | 19 |
| | Y180/F404/K42-LP-S7-1 | | | | | | |
| iADC 13 | h1925-C06 | 0.39 | 11,572 | NC | <5 | 0.088 | 46 |
| | Y180/F404-LP-C1/K42-LP-S7-1 | | | | | | |
| iADC 14 | h1925-C06 | 0.18 | 11,503 | 0.36 | 19.8 | 0.065 | 37 |
| | Y180/F404-LP-C1/K42-LP-S9-1 | | | | | | |
| iSAC 12 | h1925-C06 | NC | <5000 | NC | <5 | 2.714 | 16 |
| | Y180/F404/K42-LP-S3-1 | | | | | | |
| iADC 15 | h1925-C06 | 0.42 | 11,042 | NC | <5 | 0.255 | 43 |
| | Y180/F404-LP-C1/K42-LP-S3-1 | | | | | | |
| iSAC 13 | h1925-C06 | 0.74 | 13257 | 0.46 | 13.7 | 0.097 | 20 |
| | Y180/F404/K42-LP-S3-2 | | | | | | |
| iADC 16 | h1925-C06 | 0.60 | 12,218 | 2.36 | 14.1 | 0.332 | 30 |
| | Y180/F404-LP-C1/K42-LP-S3-2 | | | | | | |
| iSAC 14 | h1925-C06 | NC | <5000 | 26.22 | 17.9 | 0.094 | 10 |
| | Y180/F404/K42-LP-S3-3 | | | | | | |
| iADC 17 | h1925-C06 | 1.17 | 12,280 | NC | <5 | 2.115 | 23 |
| | Y180/F404-LP-C1/K42-LP-S3-3 | | | | | | |
| iSAC 16 | h1925-C06 | 1.05 | 15,566 | 1.64 | 15.6 | 0.922 | 27 |
| | Y180/F404/K42-LP-S3-5 | | | | | | |
| iADC 19 | h1925-C06 | 0.52 | 12,973 | 2.03 | 16.5 | 0.406 | 26 |
| | Y180/F404-LP-C1/K42-LP-S3-5 | | | | | | |
| iSAC20 | h1925-C06 | 0.100 | 16,114 | 0.28 | 15.7 | 0.074 | 27 |
| | Y180/F404/K42-LP-S3-4 | | | | | | |
| iADC 44 | h1925-C06 | 0.028 | 27,622 | 0.73 | 15.1 | 0.119 | 28 |
| | Y180/F404-LP-C1/K42-LP-S3-4 | | | | | | |

NC = Not Calculable due to incomplete curve

Table 12 summarizes the data for TROP2 iADCs conjugated to STING agonist LPs LP-S-11-1, LP-S12-1, LP-S13-1, LP-S13-2, LP-S3-5, LP-S14-1, LP-S14-2, LP-S-15-1 in a co-culture assay of NCI-1H292 cells and human PBMCs. All the iADCs induced similar or slightly more potent tumor cell killing than the ADC 4. TROP2 iADC 38 and iADC 39 did not induce any immune cell activation. iADC 2 is still the most potent iADC in activating different immune cells, followed by iADC 36, iADC 37, iADC 40, iADC 20, iADC 41, and iADC 42 (only monocyte activation is shown here, similar trend observed for DC, B cell, T cell and NK cell activation).

TABLE 12
TROP2 ADC, iSACs and iADCs induced tumor cell killing and immune cell activation

| | | NCI-H292 | | Monocyte | | | |
|---|---|---|---|---|---|---|---|
| | | Cell Count | | Cell Count | | Monocyte | |
| | | | Span | | Span | Activation | |
| ADC or | | EC₅₀ | (cell | EC₅₀ | (cell | EC₅₀ | Span |
| iADC # | Description | (nM) | count) | (nM) | count) | (nM) | (%) |
| ADC 4 | h1925-C06 | 0.28 | 11,044 | >100 | NC | >100 | NC |
| | Y180/F404-LP-C1/K42 | | | | | | |
| iADC 2 | h1925-C06 | 0.05 | 8,511 | 0.04 | 25.49 | 0.026 | 20.23 |
| | Y180/F404-LP-C1/K42-LP-S1-2 | | | | | | |
| iADC 36 | h1925-C06 | 0.06 | 12,971 | 0.38 | 14.89 | 0.308 | 34.14 |
| | Y180/F404-LP-C1/K42-LP-S11-1 | | | | | | |
| iADC 37 | h1925-C06 | 0.07 | 13,194 | 0.35 | 18.82 | 1.343 | 17.55 |
| | Y180/F404-LP-C1/K42-LP-S12-1 | | | | | | |
| iADC 38 | h1925-C06 | 0.16 | 10,339 | >100 | NC | >100 | NC |
| | Y180/F404-LP-C1/K42-LP-S-13-1 | | | | | | |
| iADC 39 | h1925-C06 | 0.13 | 11,207 | >100 | NC | >100 | NC |
| | Y180/F404-LP-C1/K42-LP-S-13-2 | | | | | | |
| iADC 20 | h1925-C06 | 0.46 | 8,267 | 0.76 | 16.8 | 0.918 | 21.96 |
| | Y180/F404-LP-C1/K42-LP-S3-5 | | | | | | |
| iADC 40 | h1925-C06 | 0.09 | 8,218 | 0.36 | 21.86 | 0.053 | 20.35 |
| | Y180/F404-LP-C1/K42-LP-S14-1 | | | | | | |
| iADC 41 | h1925-C06 | 0.08 | 10,165 | 1.77 | 20.26 | 0.894 | 18.87 |
| | Y180/F404-LP-C1/K42-LP-S-14-2 | | | | | | |
| iADC 42 | h1925-C06 | 0.12 | 9,723 | 1.75 | 28.97 | 0.56 | 26.54 |
| | Y180/F404-LP-C1/K42-LP-S-15-1 | | | | | | |

NC = Not Calculable due to incomplete curve

### Example 8. TROP2 iADCs Conjugated to Different Cytotoxins and STING Agonists with Different Linkers in Co-Culture Assays

TROP2 iADCs conjugated to either LP-C1 or LP-C2 and different STING agonists LPs were tested in vitro co-culture assay with TROP2 positive NCI-1H292 cells and human PBMCs.

As shown in Table 13, TROP2 iADC conjugated to LP-C1 or LP-C2 induced similar potent cell killing on the tumor cells. When conjugated to the same STING LP, TROP2 iADCs conjugated to LP-C1 induced more potent immune cell activation than iADCs conjugated to LP-C2 (only monocyte activation is shown here, similar trend observed for DC, B cell, T cell and NK cell activation).

TABLE 13
TROP2 ADC, iSACs and iADCs induced tumor cell killing and immune cell activation

| | | NCI-H292 | | Monocyte | | | |
|---|---|---|---|---|---|---|---|
| | | Cell Count | | Cell Count | | Monocyte | |
| | | | Span | | Span | Activation | |
| iSAC or | | IC₅₀ | (cell | IC₅₀ | (cell | EC₅₀ | Span |
| iADC | Description | (nM) | count) | (nM) | count) | (nM) | (%) |
| ADC 4 | h1925-C06 | 1.67 | 4,575 | 32.55 | 1.282 | 6.546 | 2.32 |
| | Y180/F404-LP-C1/K42 | | | | | | |
| iADC 1 | h1925-C06 | 2.70 | 9,228 | 0.081 | 22.77 | 0.26 | 8.506 |
| | Y180/F404-LP-C1/K42-LP-S1-1 | | | | | | |
| iADC 43 | h1925-C06 | 1.11 | 6,148 | 0.089 | 23.72 | 0.443 | 8.894 |
| | Y180/F404-LP-C2/K42-LP-S1-1 | | | | | | |
| iADC 4 | h1925-C06 | 2.37 | 4,676 | 0.022 | 23.09 | 0.016 | 4.77 |
| | Y180/F404-LP-C1/K42-LP-S4-1 | | | | | | |
| iADC 12 | h1925-C06 | 1.02 | 5,588 | 0.088 | 23.33 | 0.23 | 8.473 |
| | Y180/F404-LP-C2/K42-LP-S4-1 | | | | | | |

Table 14 summarized data for TROP2 ADC, iSAC and iADCs conjugated to LP-C1 (exatecan), LP-C5 (cleavable MMAE) or LP-C3 (non-cleavable maytansinoid) and/or LP-S1-1 (non-cleavable Compound S-1) or LP-S1-2 (cleavable S Compound S-1). All iADCs induced similar potent cell killing on the tumor cells. When conjugated to the same STING LP, TROP2 iADCs conjugated to the three different cytotoxic LPs induced similar immune cell activation. When conjugated to the same cytotoxic LP, TROP2 iADCs conjugated to cleavable STING-LP LP-S1-2 induced slightly more potent immune cell activation than TROP2 iADCs conjugated to non-cleavable STING-LP-S1-1 (only monocyte activation is shown here, similar trend observed for DC, B cell, T cell and NK cell activation).

TABLE 14
TROP2 ADC, iSACs and iADCs induced tumor cell killing and immune cell activation

| | | NCI-H292 | | Monocyte | | Monocyte | |
|---|---|---|---|---|---|---|---|
| | | Cell Count | | Cell Count | | Activation | |
| | | IC₅₀ | | IC₅₀ | | EC₅₀ | |
| SP # | Description | (nM) | Span | (nM) | Span | (nM) | Span |
| ADC 4 | h1925-C06 | 1.24 | 5,125 | >100 | NC | >100 | NC |
| | Y180/F404-LP-C1/K42 | | | | | | |
| iSAC 1 | h1925-C06 | >100 | NC | 0.078 | 14.1 | 0.177 | 5.84 |
| | Y180/F404/K42-LP-S1-1 | | | | | | |
| iADC 1 | h1925-C06 | 0.36 | 4,585 | 0.07 | 14.94 | 0.053 | 3.81 |
| | Y180/F404-LP-C1/K42-LP-S1-1 | | | | | | |
| iADC 25 | h1925-C06 | 0.32 | 6,296 | 0.098 | 13.56 | 0.037 | 3.81 |
| | Y180/F404-LP-C5/K42-LP-S1-1 | | | | | | |
| iADC 26 | h1925-C06 | 0.30 | 6,463 | 0.08 | 13.38 | 0.035 | 3.97 |
| | Y180/F404-LP-C3/K42-LP-S1-1 | | | | | | |
| iADC 2 | h1925-C06 | 0.15 | 4,834 | 0.117 | 13.29 | 0.016 | 3.36 |
| | Y180/F404-LP-C1/K42-LP-S1-2 | | | | | | |
| iADC 28 | h1925-C06 | 0.18 | 7,272 | 0.135 | 14.3 | 0.061 | 4.76 |
| | Y180/F404-LP-C5/K42LP-S1-2 | | | | | | |
| iADC 29 | h1925-C06 | 0.07 | 7,497 | 0.146 | 15.23 | 0.076 | 3.61 |
| | Y180/F404-LP-C3/K42-LP-S1-2 | | | | | | |

Table 15 summarizes data for TROP2 ADC, iSAC and iADCs conjugated to LP-C1 (beta-glucuronidase sensitive cleavable exatecan) or LP-C4 (cathepsin sensitive cleavable exatecan) and LP-S1-1 (non-cleavable Compound S-1) or LP-S1-2 (cleavable Compound S-1). All iADCs induced similar potent cell killing on the tumor cells and similar immune cell activation (only monocyte activation is shown here, similar trend observed for DC, B cell, T cell and NK cell activation).

TABLE 15
TROP2 ADC, iSACs and iADCs induced tumor cell killing and immune cell activation

| | | NCI-H292 | | Monocyte | | Monocyte | |
|---|---|---|---|---|---|---|---|
| | | Cell Count | | Cell Count | | Activation | |
| | | IC₅₀ | | IC₅₀ | | EC₅₀ | |
| SP # | Description | (nM) | Span | (nM) | Span | (nM) | Span |
| ADC 4 | h1925-C06 | 0.30 | 4,360 | >100 | NC | >100 | NC |
| | Y180/F404-LP-C1/K42 | | | | | | |
| iSAC 1 | h1925-C06 | 0.21 | 3,691 | 0.044 | 19.33 | 0.062 | 5.308 |
| | Y180/F404/K42-LP-S1-1 | | | | | | |
| iADC 1 | h1925-C06 | 0.16 | 4,098 | 0.031 | 19.69 | 0.084 | 5.436 |
| | Y180/F404-LP-C1/K42-LP-S1-1 | | | | | | |
| iADC 2 | h1925-C06 | 0.04 | 3,813 | 0.034 | 21.65 | 0.066 | 7.18 |
| | Y180/F404-LP-C1/K42-LP-S1-2 | | | | | | |
| iADC 24 | h1925-C06 | 0.04 | 4,978 | 0.038 | 19.1 | 0.052 | 4.535 |
| | Y180/F404-LP-C4/K42-LP-S1-1 | | | | | | |
| iADC 27 | h1925-C06 | 0.04 | 4,909 | 0.04 | 21.72 | 0.074 | 9.947 |
| | Y180/F404-LP-C4/K42-LP-S1-2 | | | | | | |

### Example 9. TROP2 iADCs in Co-Culture Assay with PBMCs and TROP2 Positive or Negative Tumor Cells

TROP2 iADCs conjugated to STING agonists with different potency and linkers were also tested in vitro co-culture assay with TROP2 positive NCI-H292 cells or TROP2 negative NCI-H460 cells and human PBMCs. Exatecan was added as a control. The EC₅₀ and span for target tumor cell killing, monocyte % in the PBMCs, monocyte activation and IP-10 release are summarized in Table 16 and Table 17. The Span is reported by Prism software as “the difference between the high plateau (Top) of the sigmoidal dose response curve to the low plateau (Bottom).” Similar trend in other immune cell activation were observed (CD4+ T, CD8+ T cells, NK, and B cell) and cytokine release (IFNγ, TNFα, IL-6, IL-1α and MIP-1α) but not shown.

As expected, exatecan induced potent tumor cell killing with a similar IC₅₀ on both TROP2 positive and negative cells and did not induce activation of any immune populations (Table 16 and Table 17). ADC 4 induced potent cell killing on TROP2 positive cells but not on TROP2 negative cells and did not induce much activation of any immune populations (Table 13). TROP2 iADCs induced similar or slightly more potent cell killing on TROP2 positive co-cultures compared to ADC 4. The corresponding iSACs did not induce any tumor cell killing in either TROP2 positive or negative co-cultures.

When PBMCs were co-cultured with TROP2 positive NCI-H292 cells, iADC 1 and iADC 9 showed the same activity in target cell killing, immune cell activity and cytokine release (Table 16). iADC 17 induced much less immune cell activation and cytokine release compared to iADC 1 and iADC 10, which is correlated to the lower STING activity for Compound S-3 compared to Compound S-1.

When PBMCs were co-cultured with TROP2 negative NCI-H460 cells (Table 17), iADC 1 and iADC 9 induced some activation of immune cells and some cytokine release, but with much reduced potency, which might be related non-specific uptake of the iADCs into the negative cells. iADC 17 induced no or much reduced immune cells activation and cytokine release in the PBMCs compared to iADC 1 and iADC 9 when co-cultured with TROP2 negative cells.

TABLE 16
TROP2 ADCs, iSACs and iADCs induced tumor cell killing and immune cell activation

| | | NCI-H441/PBMC co-culture | | | | | | | |
|---|---|---|---|---|---|---|---|---|---|
| | | Cancer | | | | | | | |
| | | Cell Count | | Monocyte | | Monocyte | | | |
| ADC | | | Span | Cell Count | | Activation | | IP-10 release | |
| and | | IC₅₀ | (Cell | IC₅₀ | Span | EC₅₀ | Span | EC₅₀ | Span |
| iADC # | Description | (nM) | Count) | (nM) | (%) | (nM) | (%) | (nM) | (pg/mL) |
| | Exatecan free drug | 0.44 | 5,749 | NC | NC | NC | NC | NC | NC |
| ADC 4 | h1925-C06 | 0.37 | 2,784 | NC | NC | NC | NC | NC | NC |
| | Y180/F404-LP-C1/K42 | | | | | | | | |
| ADC 6 | h1925-C06 | NC | NC | NC | NC | NC | NC | NC | NC |
| | F241/F404-LP-C1/K42 | | | | | | | | |
| iSAC 1 | h1925-C06 | NC | NC | 0.05 | 20.00 | 0.09 | 6.80 | 0.14 | 36,451 |
| | Y180/F404/K42-LP-S1-1 | | | | | | | | |
| iADC 1 | h1925-C06 | 0.13 | 6,049 | 0.06 | 19.00 | 0.07 | 5.40 | 0.22 | 29,058 |
| | Y180/F404-LP-C1/K42-LP-S1-1 | | | | | | | | |
| iADC 9 | h1925-C06 | 0.12 | 6,254 | 0.05 | 19.00 | 0.07 | 5.40 | 0.28 | 25,910 |
| | F241/F404-LP-C1/K42-LP-S1-1 | | | | | | | | |
| iSAC 2 | h1925-C06 | NC | NC | 0.05 | 20.00 | 0.07 | 6.40 | 0.19 | 32,480 |
| | Y180/F404/K42-LP-S2-1 | | | | | | | | |
| iSAC 14 | h1925-C06 | NC | NC | 0.15 | 13.00 | 2.90 | 2.40 | 9.12 | 14,062 |
| | Y180/F404/K42-LP-S3-3 | | | | | | | | |
| iADC 17 | h1925-C06 | 0.26 | 5,543 | 0.18 | 12.00 | 3.51 | 1.60 | 1.29 | 2,608 |
| | Y180/F404-LP-C1/K42-LP-S3-3 | | | | | | | | |

NC = Not Calculable due to incomplete curve

TABLE 17
TROP2 ADC, iSACs and iADCs induced tumor cell killing,
immune cell activation and cytokine release

| | | NCI-H460/PBMC co-culture | | | | | | | |
|---|---|---|---|---|---|---|---|---|---|
| | | Cancer | | | | | | | |
| | | Cell Count | | Monocyte | | Monocyte | | | |
| ADC | | | Span | Cell Count | | Activation | | IP-10 release | |
| and | | IC50 | (Cell | IC₅₀ | Span | EC₅₀ | Span | EC₅₀ | Span |
| iADC # | Description | (nM) | Count) | (nM) | (%) | (nM) | (%) | (nM) | (pg/mL) |
| | Exatecan | 0.38 | 23,530 | NC | NC | NC | NC | NC | NC |
| ADC 4 | h1925-C06 | NC | NC | NC | NC | NC | NC | NC | NC |
| | Y180/F404 LP-C1/K42 | | | | | | | | |
| ADC 6 | h1925-C06 | NC | NC | NC | NC | NC | NC | NC | NC |
| | F241/F404-LP-C1/K42 | | | | | | | | |
| iSAC 1 | h1925-C06 | NC | NC | NC | NC | 0.69 | 1.70 | NC | NC |
| | Y180/F404/K42-LP-S1-1 | | | | | | | | |
| iADC 1 | h1925-C06 | NC | NC | 2.45 | 21.00 | 0.38 | 1.40 | NC | NC |
| | Y180/F404-LP-C1/K42-LP-S1-1 | | | | | | | | |
| iADC 9 | h1925-C06 | NC | NC | 3.29 | 24.00 | 0.44 | 1.50 | NC | NC |
| | F241/F404-LP-C1/K42-LP-S1-1 | | | | | | | | |
| iSAC 2 | h1925-C06 | NC | NC | NC | NC | 0.40 | 1.40 | NC | NC |
| | Y180/F404/K42-LP-S2-1 | | | | | | | | |
| iSAC 14 | h1925-C06 | NC | NC | NC | NC | NC | NC | NC | NC |
| | Y180/F404/K42-LP-S3-3 | | | | | | | | |
| iADC 17 | h1925-C06 | NC | NC | NC | NC | NC | NC | NC | NC |
| | Y180/F404-LP-C1/K42-LP-S3-3 | | | | | | | | |

NC = Not Calculable due to incomplete curve

### Example 10. ISACs and iADC Induced Cytokine Release in Cancer Cells

To evaluate if TROP2 ADCs, iSACs and iADCs treatment will result in cytokine release in TROP2 positive cells, NCI-H292 was treated with test articles for 120 hours. The cell supernatant was collected and IP-10 and IL-6 released in the cell culture medium was measured using Streptavidin single spot 96 well plates, compatible human antibody pairs, and calibrating solutions commercially available from MesoScale Discovery. Raw data generated by the Meso Scale Discovery (MSD®) SQ120 plate reader was imported into MSD Discovery Workbench software. Unknown sample values were interpolated from standard curves by the software and imported into Graphpad Prism. Each cytokine level was fitted with non-linear regression analysis, using log(agonist) vs. response, variable slope, 4-parameter fit equation in GraphPad Prism.

As shown in Table 18, STING agonists Compound S-1 and Compound S-3, but not exatecan, were able to induce IP-10 and IL-6 release from NCI-H292 cells at EC₅₀ values greater than 100 nM. TROP2 ADC 4 did not induce IP-10 or IL-6 above the baseline of untreated cells. iSAC 1, iSAC 2, iSAC 14, iADC 1 and iADC 9, resulted in similar overall induction of IP-10 (EC₅₀˜2 nM; Span˜2000) with the exception of iADC 17 which was slighted attenuated and had a lower overall magnitude (EC₅₀˜3 nM; Span ˜760). In contrast to these findings, the magnitude of IL-6 induction by iSAC 1, iSAC 2, and iSAC 14 (EC₅₀ 2-4 nM; Span˜6000) was approximately three-fold greater than corresponding iADC 1, iADC 9, and iADC 17 (EC₅₀˜0.2-0.4 nM; Span ˜2000), which correlated to the fact that the iSACs are not cytotoxic while the ADCs and iADCs are. These results indicate that release of IP-10 and IL-6 are primarily dictated by STING agonist activity for iSACs and iADCs.

TABLE 18
TROP2 iSACs and iADCs induced cytokine release
on TROP2 positive NCI-H292 Cells

| | | IP-10 Release | | IL-6 Release | |
|---|---|---|---|---|---|
| ADC or | | EC₅₀ | Span | EC₅₀ | Span |
| iADC # | Description | (nM) | (pg/mL) | (nM) | (pg/mL) |
| | Exatecan | 0.87 | 203 | 14.69 | 1410 |
| Compound | STING agonist | >100 | >4000 | >100 | >10000 |
| S-1 | | | | | |
| Compound | STING agonist | >100 | >4000 | >100 | >8000 |
| S-3 | | | | | |
| ADC 4 | h1925-C06 Y180/F404-LP- | NC | NC | NC | NC |
| | C1/K42 | | | | |
| iSAC 1 | h1925-C06 Y180/F404/K42-LP- | 2.91 | 2351 | 3.8 | 6987 |
| | S1-1 | | | | |
| iADC 1 | h1925-C06 Y180/F404-LP- | 2.05 | 2345 | 0.44 | 2452 |
| | C1/K42- LP-S1-1 | | | | |
| iADC 9 | h1925-C06 F241/F404-LP- | 2.58 | 1994 | 0.32 | 2063 |
| | C1/K42- LP-S1-1 | | | | |
| iSAC 2 | h1925-C06 Y180/F404/K42-LP- | 1.54 | 2067 | 3.62 | 6011 |
| | S2-1 | | | | |
| iSAC 14 | h1925-C06 Y180/F404/K42-LP- | 1.59 | 1996 | 1.74 | 6790 |
| | S3-3 | | | | |
| iADC 17 | h1925-C06 Y180/F404-LP- | 2.77 | 756 | 0.2 | 2235 |
| | C1/K42- LP-S3-3 | | | | |

NC = Not Calculable due to incomplete curve

### Example 11. TROP2 ADCs and iADCs Induced Immunogenic Cell Death

Immunogenic cell death elicited by the TROP2 ADC, iSAC, iADCs and exatecan were measured by cell surface translocate of calreticulin and release of HMGB1 in the cell culture supernatant. Briefly, TROP2 positive cell line NCI-H292 were seeded into a 96-well flat bottom plate 4 hours prior to the initialization of the assay. Test articles were formulated at a 2× starting concentration in cell culture medium, and samples were serial diluted (1:3) under sterile conditions and added onto the cells in duplicates. After incubation at 37° C. in a CO₂ incubator for 48 hours, the cell supernatant was collected for measuring HMGB1 and the adherent cells were collected for calreticulin expression on the cell surface.

HMGB1 released in the supernatant was assessed via sandwich ELISA using mouse anti-human HMGB1 clone 2F6 (Sigma-Aldrich) as the capture, polyclonal rabbit anti-human HMGB1 (Abcam) as secondary, and polyclonal goat anti-rabbit IgG HRP as the detection antibody. The HMGB1 concentrations in the cell culture medium were calculated based a standard curve built on commercially available recombinant human HMGB1 (R&D Systems).

NCI-H292 cells were detached using Accutase (Innovative Cell Technologies) and viability was accessed using a 1:500 dilution of Zombie NIR viability dye. Calreticulin induction was measured via commercial anti-calreticulin antibody conjugated to Alexa Fluor 647 (Abcam) diluted at 1:60. Surface calreticulin measures on the live cells were gathered using an Attune NXT Flow Cytometer (ThermoFisher Scientific) to obtain median fluorescence intensity values. All data were fitted with non-linear regression analysis, using log(agonist) vs. response, variable slope, 4 parameter fit equation using GraphPad Prism.

As shown in Table 19, exatecan, ADC 4 and iADCs (iADC 1, iADC 9, iADC17) resulted in significant and overall similar release of HMGB1 (˜70 ng/mL) into the cell culture supernatant and a ˜2-fold induction of CALR on live NCI-H292 cells when compared to untreated cells. Free STING agonists Compound S-1 and Compound S-3, and the corresponding iSACs (iSAC 1, iSAC 2, and iSAC 14) resulted in much reduced release of HMGB1 ((30 ng/mL) and no induction of CALR when compared to untreated control cells. These data indicate that immunogenic cell death is primarily driven by exatecan and not STING agonist Compound S-1 or Compound S-3, although the STING agonists may be sufficient to induce cell stress given HMGB1 release and lack of CALR induction.

TABLE 19
TROP2 iADCs induced immunogenic cell
death markers on NCI-H292 cells

| ADC | | | | | |
|---|---|---|---|---|---|
| or | | HMGB-1 Release | | Calreticulin | |
| iADC | | EC₅₀ | Span | EC₅₀ | Span |
| # | Description | (nM) | (ng/mL) | (nM) | (MFI) |
| | Exatecan | 2.08 | 55 | 0.19 | 783 |
| Compd | STING agonist | 6.06 | 17 | NC | NC |
| S-1 | | | | | |
| Compd | STING agonist | 7.45 | 11 | NC | NC |
| S-3 | | | | | |
| ADC 4 | h1925-C06 Y180/F404-LP- | 2.38 | 47 | 0.53 | 1121 |
| | C1/K42 | | | | |
| iSAC 1 | h1925-C06 | 1.73 | 16 | NC | NC |
| | Y180/F404/K42-LP-S1-1 | | | | |
| iADC 1 | h1925-C06 Y180/F404-LP- | 2.02 | 32 | 0.21 | 1552 |
| | C1/K42-LP-S1-1 | | | | |
| iADC 9 | h1925-C06 F241/F404-LP- | 1.83 | 38 | 0.45 | 737 |
| | C1/K42-LP-S1-1 | | | | |
| iSAC 2 | h1925-C06 | 2.71 | 9 | NC | NC |
| | Y180/F404/K42-LP-S2-1 | | | | |
| iSAC | h1925-C06 | 1.29 | 16 | NC | NC |
| 14 | Y180/F404/K42-LP-S3-3 | | | | |
| iADC | h1925-C06 Y180/F404- | 2.16 | 43 | 7.42 | 1384 |
| 17 | LP-C1/K42-LP-S3-3 | | | | |

NC = Not Calculable due to incomplete curve

### Example 12. Anti-Tumor Activity of TROP2 ADCs in a Syngeneic Mouse Tumor Model

The activity of TROP2 conjugates was examined in hTrop2 engineered MC38 syngeneic mouse model (MC38-hTrop2). Briefly, C57BL/6 mice were implanted subcutaneously with 0.5×10⁶ MC38-hTrop2 tumor cells and randomized and enrolled into the study 7 days post implant, at an average tumor size of 140 mm³. Tumor-bearing mice were administered a single dose of all test articles at doses ranging from 2 mg/kg to 10 mg/kg. All treatments were well tolerated with normal body weight gain throughout the course of the study.

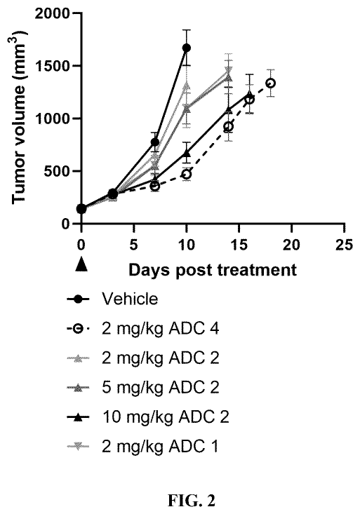

FIG. 2 illustrates the effects of ADC 4, ADC 2, and ADC 1 on tumor growth until day 18 post treatment. Analysis of tumor sizes was done on day 10, when the mean of vehicle-treated tumors reached the study endpoint (>1,500 mm3). At a dose of 2 mg/kg, ADC 4 induced greater tumor growth suppression (79% TGI) compared to ADC 1 (38% TGI) and ADC 2 (23% TGI). ADC 2 showed dose-dependent anti-tumor activity (23% TGI at 2 mg/kg, 38% TGI at 5 mg/kg, 65% TGI at 10 mg/kg).

### Example 13. Anti-Tumor Activity of TROP2 iADCs in a Syngeneic Mouse Tumor Model

C57BL/6 mice were implanted with 0.2×10⁶ MC38-hTrop2 tumor cells subcutaneously. Mice were randomized and enrolled into the study 8 days post implant, with an average tumor size of 95 mm³. Tumor-bearing mice were administered a single dose of the test articles at doses ranging from 1 mg/kg to 2 mg/kg. All treatments were well tolerated with normal body weight gain throughout the course of the study.

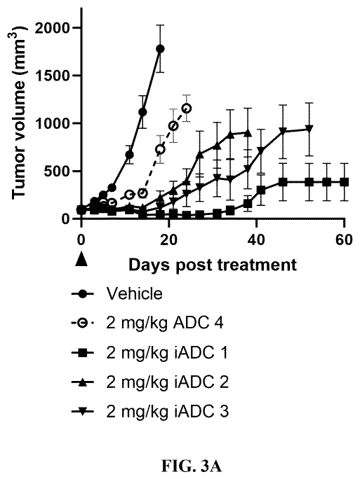

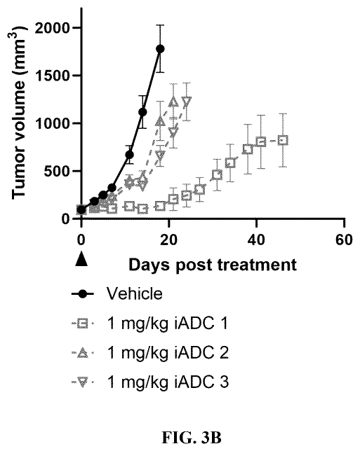

FIG. 3A and FIG. 3B summarize the efficacy of TROP2 iADCs on growth of MC38-hTrop2 tumors until day 60 post treatment. At a dose of 2 mg/kg, iADC 1, iADC 2, and iADC 3 demonstrated potent and improved anti-tumor activity (95%-100% TGI) compared to ADC 4 (62% TGI) on day 18 of the study (FIG. 3A). Treatment with 2 mg/kg iADCs 1, iADC 2, and iADC 3 also resulted in tumor free mice (70%, 30% and 30% respectively). At a dose of 1 mg/kg, all iADCs demonstrated potent tumor growth inhibition (97% TGI and 30% tumor free mice with ADC 1, 45% TGI with iADC 2 and 67% TGI with iADC 3) (FIG. 3B).

### Example 14. Anti-Tumor Activity of TROP2 iADCs in a Syngeneic Mouse Tumor Model

C57BL/6 mice were implanted with 0.2×10⁶ MC38-hTrop2 tumor cells subcutaneously. Mice were randomized and enrolled into the study 10 days post implant, with an average tumor size of 99 mm³. Tumor-bearing mice were administered a single dose of the test articles at doses ranging from 1 mg/kg to 2 mg/kg. All treatments were well tolerated with normal body weight gain throughout the course of the study.

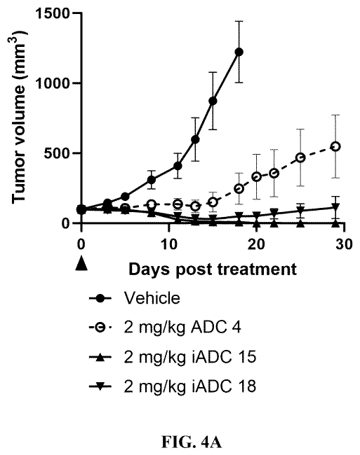

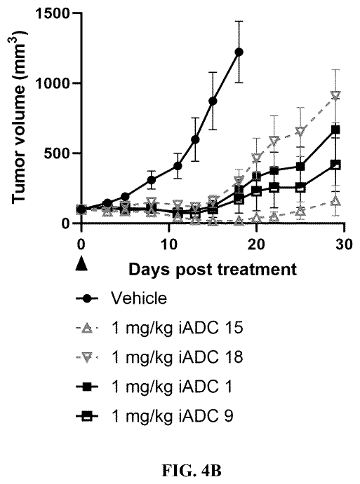

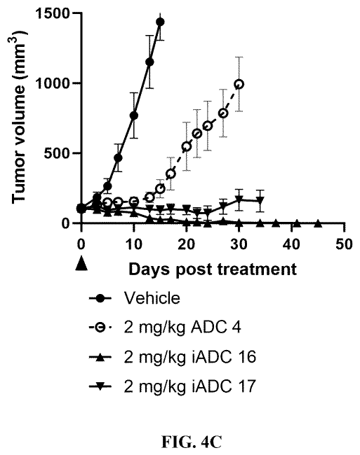

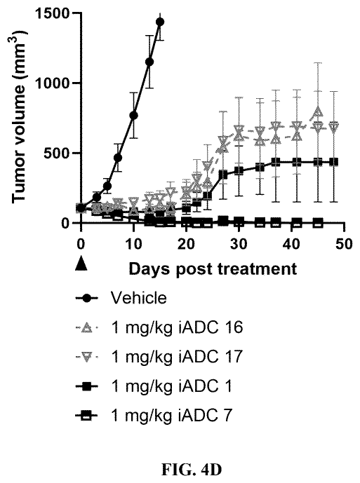

FIG. 4A and FIG. 4B summarize the efficacy of TROP2 iADC on growth of MC38-hTrop2 tumors. Analysis of treatment groups was done on day 18, when the mean of vehicle-treated tumors reached the study endpoint (>1,500 mm³). At a dose of 2 mg/kg, iADC 15 and iADC 18 demonstrated potent and improved anti-tumor activity compared to ADC 4 (TGI 108%, 104%, and 87% respectively) (FIG. 4A). Treatment with both iADCs at the 2 mg/kg dose resulted in tumor free animals (80% with iADC 18 and 100% with iADC 15). Analysis of anti-tumor activity at 1 mg/kg dose revealed that iADCs with all linker payloads induced potent activity and a range of tumor free mice (iADC 15: 107% TGI, 80% tumor free mice; iADC 18: 82% TGI, 20% tumor free mice; iADC 1: 88% TGI, 40% tumor free mice; iADC 9: 94% TGI, 60% tumor free mice) (FIG. 4B)

Results from additional studies are summarized in FIG. 4C and FIG. 4D. Analysis of treatment groups was done when the mean of vehicle-treated tumors reached the study endpoint (>1,500 mm³) on day 15. At a dose of 2 mg/kg, iADC 16 and iADC 17 significantly improved anti-tumor activity compared to ADC 4 (TGI 106%, 101% and 89% respectively) (FIG. 4C). Treatment with both iADCs at the 2 mg/kg dose resulted in tumor free animals (iADC 16: 100%; iADC 17: 60%). Analysis of efficacy at 1 mg/kg dose revealed that all iADCs, induced potent efficacy and a range of tumor free mice (iADC 16: 98% TGI, 60% tumor free mice; iADC 17: 95% TGI, 50% tumor free mice; iADC 1: 102% TGI, 50% tumor free mice; iADC 7: 107% TGI, 100% tumor free mice) (FIG. 4D)

### Example 15. Anti-Tumor Activity of TROP2 iADCs & iSACs in a Syngeneic Mouse Tumor Model

C57BL/6 mice were implanted with 0.2×10⁶ MC38-hTrop2 tumor cells subcutaneously. Mice were randomized and enrolled into the study 8 days post implant, with tumor sizes around 100-115 mm³. Tumor-bearing mice were administered a single dose of the test articles at doses ranging from 0.5 mg/kg to 1 mg/kg. All treatments were well tolerated with normal body weight gain throughout the course of the study.

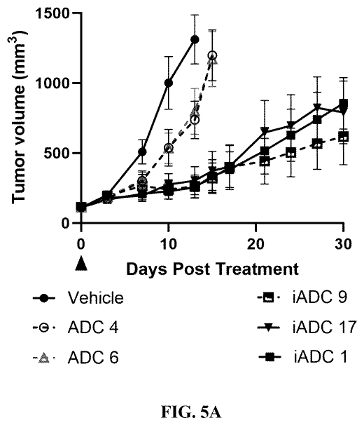

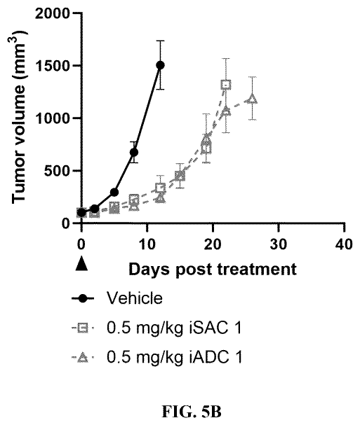

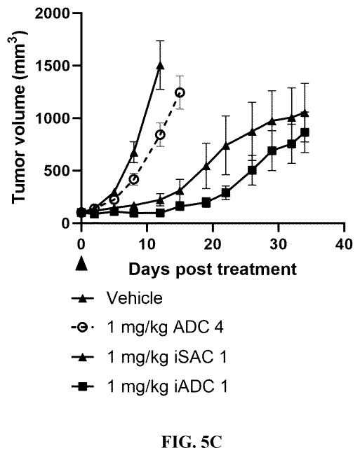

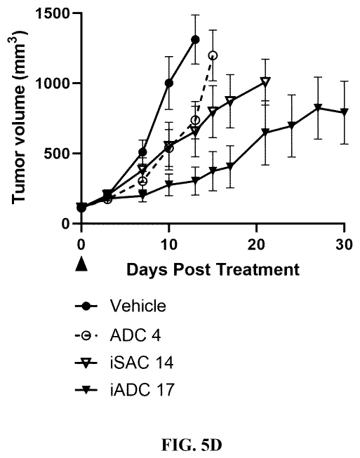

FIG. 5A-FIG. 5E summarize effects of TROP2 conjugates with and without STING agonist on growth of MC38-hTrop2 tumors. Analysis of treatment groups was done on day 12, when the mean of vehicle-treated tumors reached the study endpoint (>1,500 mm³). At a dose of 1 mg/kg, ADC 4 and ADC 6 induced comparable anti-tumor activity (48% and 43% TGI respectively). In comparison, 1 mg/kg of TROP2 iADCs, regardless of STING linker payload, induced potent and comparable anti-tumor activity (iADC 1: 88% TGI, iADC 9: 88% TGI, iADC 17: 84% TGI) and greater anti-tumor activity compared to the ADCs (FIG. 5A). iSAC 1 and iADC 1 demonstrated comparable anti-tumor activity (80%-100% TGI) (FIG. 5B) at both 0.5 mg/kg and 1 mg/kg. (FIG. 5C). At both 0.5 mg/kg and 1 mg/kg, treatment with TROP2 conjugates with immune agonists also resulted in 10-20% tumor free mice. At a 1 mg/kg dose iADC 17 (84% TGI) showed improved anti-tumor activity compared to ADC 4 (48% TGI) and iSAC 14 (55% TGI) (FIG. 5D). iADC 9 (88% TGI) slowed tumor progression compared to iSAC 2 (86% TGI), and improved anti-tumor activity compared to ADC 6 (43% TGI) (FIG. 5E).

### Example 16: TROP2 iADCs Induce Immune Cell Activation in Plasma and Tumor Microenvironment

The effect of TROP2-STING iADCs on the activation of immune responses was evaluated in the MC38-hTrop2 syngeneic mouse tumor model. Briefly, MC38-hTrop2 tumor bearing mice were administered a single dose of vehicle or 1 mg/kg TROP2 iADCs with different STING agonists intravenously.

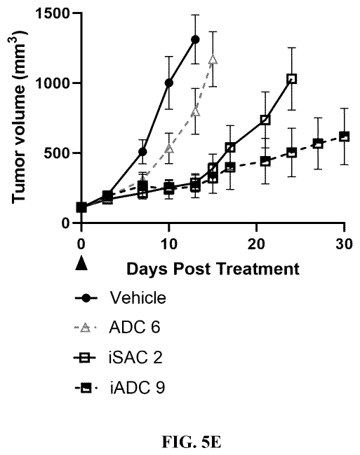

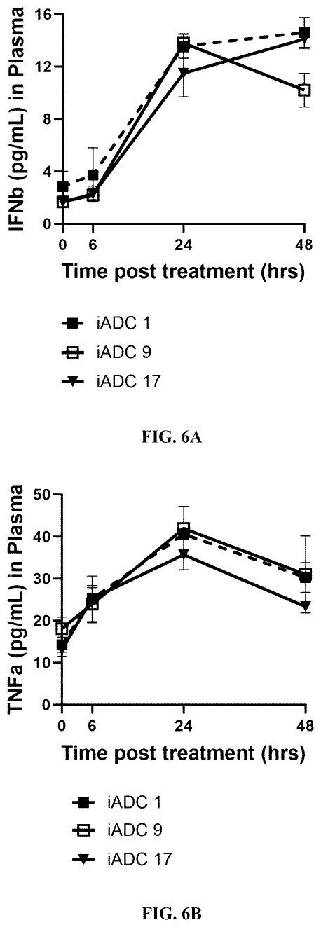

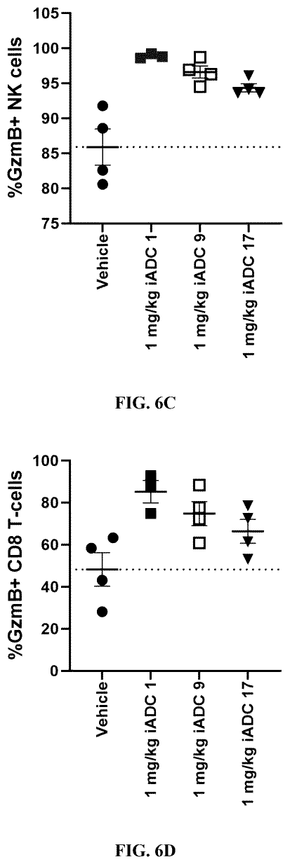

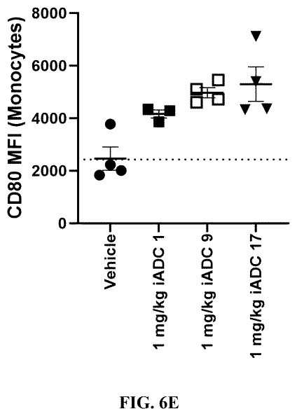

Plasma samples were analyzed for cytokine induction at multiple timepoints post treatment. Treatment with all iADCs increased systemic levels of pro-inflammatory cytokines (FIG. 6A, FIG. 6B). Effects of iADCs the of tumor microenvironment (TME) were evaluated on day 5 post treatment. Analysis of tumor infiltrating lymphocytes revealed that iADC treatment at 1 mg/kg increased the percentage of GranzymeB positive CD8-T and NK cells, compared to vehicle-treated tumors (FIG. 6C, FIG. 6D). iADC treatment also increased activation of innate immune cells, including monocytes (FIG. 6E)

### Example 17. Sequence ID Nos

| SEQ ID | | | | | |
|---|---|---|---|---|---|
| NO | Antibody # | Molecule | Region | Scheme | Sequence |
| 1 | | Human | | | MARGPGLAPPPLRLPLLLLVLAAVTGHTAAQ |
| | | Trop2 | | | DNCTCPTNKMTVCSPDGPGGRCQCRALGSGM |
| | | UniProt | | | AVDCSTLTSKCLLLKARMSAPKNARTLVRPS |
| | | P09758 | | | EHALVDNDGLYDPDCDPEGRFKARQCNQTSV |
| | | (aa 1-26 is | | | CWCVNSVGVRRTDKGDLSLRCDELVRTHHIL |
| | | signal | | | IDLRHRPTAGAFNHSDLDAELRRLFRERYRL |
| | | peptide) | | | HPKFVAAVHYEQPTIQIELRQNTSQKAAGDV |
| | | | | | DIGDAAYYFERDIKGESLFQGRGGLDLRVRG |
| | | | | | EPLQVERTLIYYLDEIPPKFSMKRLTAG |
| | | | | | LIAVIVVVVVALVAGMAVLVITNRRKSGKYK |
| | | | | | KVEIKELGELRKEPSL |
| 2 | | Cynomolgus | | | MARGPGLAPPPLRLPLLLLLLAAVTGHTAAQ |
| | | Trop2 | | | DNCTCPTNKMTVCSPDGPGGRCQCRALGSGV |
| | | NCBI | | | AVDCSTLTSKCLLLKARMSAPKNARTLVRPN |
| | | XP_00554 | | | EHALVDNDGLYDPDCDPEGRFKARQCNQTSV |
| | | 3292 (aa | | | CWCVNSVGVRRTDKGDLSLRCDELVRTHHIL |
| | | 1-26 is | | | IDLRHRPTASAFNHSDLDAELRRLFRERYRL |
| | | signal | | | HPKFVAAVHYEQPTIQIELRQNTSQKAAGDV |
| | | peptide) | | | DIGDAAYYFERDVKGESLFQGRGGLDLRVRG |
| | | | | | EPLQVERTLIYYLDEIPPKFSMKRLTAGLI |
| | | | | | AVIVVVVVALVAGVAVLVISNRRKSGKYKKV |
| | | | | | EIKELGELRKEPSL |
| 3 | | Murine | | | MARGLDLAPLLLLLLAMATRFCTAQSNCTCP |
| | | Trop2 | | | TNKMTVCDTNGPGGVCQCRAMGSQVLVDCST |
| | | UniProt | | | LTSKCLLLKARMSARKSGRSLVMPSEHAILD |
| | | Q8BGV3 | | | NDGLYDPECDDKGRFKARQCNQTSVCWCVNS |
| | | (aa 1- 24 | | | VGVRRTDKGDQSLRCDEVVRTHHILIELRHR |
| | | is signal | | | PTDRAFNHSDLDSELRRLFQERYKLHPSFLS |
| | | peptide) | | | AVHYEEPTIQIELRQNASQKGLRDVDIADAA |
| | | | | | YYFERDIKGESLEMGRRGLDVQVRGEPLHVE |
| | | | | | RTLIYYLDEKPPQFSMKRLTAGVIAVIA |
| | | | | | VVSVAVVAGVVVLVVTKRRKSGKYKKVELKE |
| | | | | | LGEMRSEPSL |
| 4 | Ab1-HC | HC_aTrop2_ | HCDR1 | Kabat | SNGIS |
| 5 | Ab2-HC | h1925- | HCDR2 | Kabat | VIDSYGTTYYADSVKG |
| 6 | | C06 | HCDR3 | Kabat | DLSYSSVMGYGMDP |
| 7 | Ab1-LC | LC_aTrop2_ | LCDR1 | Kabat | RASQSINNYLA |
| 8 | Ab2-LC | h1925- | LCDR2 | Kabat | SASYLAS |
| 9 | | C06 | LCDR3 | Kabat | QSTAGISDND |
| 10 | Ab1-HC | HC_aTrop2_ | VH | Kabat | MQVQLVESGGGVVQPGRSLRLSCAVSGFSLS |
| | Ab2-HC | h1925- | | | SNGISWVRQAPGKGLEWVAVIDSYGTTYYAD |
| | | C06 | | | SVKGRFTISRDNSKHTVYLQMNSLRAEDTAV |
| | | | | | YYCARDLSYSSVMGYGMDPWGQGTLVTVSS |
| 11 | Ab1-LC | LC_aTrop2_ | VL | Kabat | MDIQMTQSPSSLSASVGDRVTITCRASQSIN |
| | Ab2-LC | h1925- | | | NYLAWYQQKPG[pAcF]APKLLIYSASYLAS |
| | | C06 | | | GVPSRFSGSGSGTDFTLTISSLQPEDFATYY |
| | | | | | CQSTAGISDNDFGGGTKVEIKR |
| 12 | Ab1-HC | HC_aTrop2_ | HC | | MQVQLVESGGGVVQPGRSLRLSCAVSGFSLS |
| | | h1925- | | | SNGISWVRQAPGKGLEWVAVIDSYGTTYYAD |
| | | C06_Y180/ | | | SVKGRFTISRDNSKHTVYLQMNSLRAEDTAV |
| | | F404_K42 | | | YYCARDLSYSSVMGYGMDPWGQGTLVTVSSA |
| | | | | | STKGPSVFPLAPSSKSTSGGTAALGCLVKDY |
| | | | | | FPEPVTVSWNSGALTSGVHTFPAVLQSSGL |
| | | | | | [pAMF]SLSSVVTVPSSSLGTQTYICNVNHKP |
| | | | | | SNTKVDKKVEPKSCDKTHTCPPCPAPELLGG |
| | | | | | PSVFLFPPKPKDTLMISRTPEVTCVVSDVSH |
| | | | | | EDPEVKFNWYVDGVEVHNAKTKPREEQYNST |
| | | | | | YRVVSVLTVLHQDWLNGKEYKCKVSNKALPA |
| | | | | | PIEKTISKAKGQPREPQVYTLPPS |
| | | | | | REEMTKNQVSLTCLVKGFYPSDIAVEWESNG |
| | | | | | QPENNYKTTPPVLDSDGS[pAMF]FLYSKLT |
| | | | | | VDKSRWQQGNVFSCSVMHEALHNHYTOKSLS |
| | | | | | LSPGK |
| 13 | Ab2-HC | HC_aTrop2_ | HC | | MQVQLVESGGGVVQPGRSLRLSCAVSGFSLS |
| | | h1925- | | | SNGISWVRQAPGKGLEWVAVIDSYGTTYYAD |
| | | C06_F241/ | | | SVKGRFTISRDNSKHTVYLQMNSLRAEDTAV |
| | | F404_K42 | | | YYCARDLSYSSVMGYGMDPWGQGTLVTVSSA |
| | | | | | STKGPSVFPLAPSSKSTSGGTAALGCLVKDY |
| | | | | | FPEPVTVSWNSGALTSGVHTFPAVLQSSGLY |
| | | | | | SLSSVVTVPSSSLGTQTYICNVNHKPSNTKV |
| | | | | | DKKVEPKSCDKTHTCPPCPAPELLGGPSV |
| | | | | | [pAMF]LFPPKPKDTLMISRTPEVTCVVSDVSH |
| | | | | | EDPEVKFNWYVDGVEVHNAKTKPREEQYNST |
| | | | | | YRVVSVLTVLHQDWLNGKEYKCKVSNKALPA |
| | | | | | PIEKTISKAKGQPREPQVYTLPPSREEMTKN |
| | | | | | QVSLTCLVKGFYPSDIAVEWESNGQPENNYK |
| | | | | | TTPPVLDSDGS[pAMF]FLYSKLTVDKSRWQ |
| | | | | | QGNVFSCSVMHEALHNHYTQKSLSLSPGK |
| 14 | Ab1-LC | LC_aTrop2_ | LC | | MDIQMTQSPSSLSASVGDRVTITCRASQSIN |
| | Ab2-LC | h1925- | | | NYLAWYQQKPG[pAcF]APKLLIYSASYLAS |
| | | C06_Y180/ | | | GVPSRFSGSGSGTDFTLTISSLQPEDFATYY |
| | | F404_K42 | | | CQSTAGISDNDFGGGTKVEIKRTVAAPSVFI |
| | | | | | FPPSDEQLKSGTASVVCLLNNFYPREAKVQW |
| | | | | | KVDNALQSGNSQESVTEQDSKDSTYSLSSTL |
| | | | | | TLSKADYEKHKVYACEVTHQGLSSPVTKSFN |
| | | | | | RGEC |
| 15 | Ab3-HC | HC_aTrop2_ | HC | | MEVQLVESGGGLVQPGGSLRLSCAASGFNIS |
| | | 2293- | | | NKWIHWVRQAPGKGLEWVGAIAPSSGDTDYA |
| | | C04_W50A, | | | DSVKGRFTISADTSKNTAYLQMNSLRAEDTA |
| | | W103A_ | | | VYYCAREGYNAYWYVLDYWGQGTLVTVSSAS |
| | | Y180/F404_ | | | TKGPSVFPLAPSSKSTSGGTAALGCLVKDYF |
| | | K42 | | | PEPVTVSWNSGALTSGVHTFPAVLQSSGL[p |
| | | | | | AMF]SLSSVVTVPSSSLGTQTYICNVNHKPS |
| | | | | | NTKVDKKVEPKSCDKTHTCPPCPAPELLGGP |
| | | | | | SVFLFPPKPKDTLMISRTPEVTCVVSDVSHE |
| | | | | | DPEVKFNWYVDGVEVHNAKTKPREEQYNSTY |
| | | | | | RVVSVLTVLHQDWLNGKEYKCKVSNKALPAP |
| | | | | | IEKTISKAKGQPREPQVYTLPPSREEMTKNQ |
| | | | | | VSLTCLVKGFYPSDIAVEWESNGQPENNYKT |
| | | | | | TPPVLDSDGS[pAMF]FLYSKLTVDKSRWQQ |
| | | | | | GNVFSCSVMHEALHNHYTQKSLSLSPGK |
| 16 | Ab4-HC | HC_aTrop2_ | HC | | MEVQLVESGGGLVQPGGSLRLSCAASGFNIS |
| | | 2293- | | | NKWIHWVRQAPGKGLEWVGWIAPSSGDTDYA |
| | | C04_W103A_ | | | DSVKGRFTISADTSKNTAYLQMNSLRAEDTA |
| | | Y180/ | | | VYYCAREGYNAYWYVLDYWGQGTLVTVSSAS |
| | | F404_K42 | | | TKGPSVFPLAPSSKSTSGGTAALGCLVKDYF |
| | | | | | PEPVTVSWNSGALTSGVHTFPAVLQSSGL |
| | | | | | [pAMF]SLSSVVTVPSSSLGTQTYICNVNHKPS |
| | | | | | NTKVDKKVEPKSCDKTHTCPPCPAPELLGGP |
| | | | | | SVFLFPPKPKDTLMISRTPEVTCVVSDVSHE |
| | | | | | DPEVKFNWYVDGVEVHNAKTKPREEQYNSTY |
| | | | | | RVVSVLTVLHQDWLNGKEYKCKVSNKALPAP |
| | | | | | IEKTISKAKGQPREPQVYTLPPSREEMTKNQ |
| | | | | | VSLTCLVKGFYPSDIAVEWESNGQPENNYKT |
| | | | | | TPPVLDSDGS[pAMF]FLYSKLTVDKSRWQQ |
| | | | | | GNVFSCSVMHEALHNHYTQKSLSLSPGK |
| 17 | Ab3-LC | LC_aTrop2_ | LC | | MDIQMTQSPSSLSASVGDRVTITCRASQDVN |
| | Ab4-LC | 2293- | | | TAVAWYQQKPG[pAcF]APKLLIYSASFLYS |
| | | C04- | | | GVPSRFSGSRSGTDFTLTISSLQPEDFATYY |
| | | Hc_W50A, | | | CQQHYTTPPTFGQGTKVEIKRTVAAPSVFIF |
| | | W103A_ | | | PPSDEQLKSGTASVVCLLNNFYPREAKVQWK |
| | | Y180/F404_ | | | VDNALQSGNSQESVTEQDSKDSTYSLSSTLT |
| | | K42 | | | LSKADYEKHKVYACEVTHQGLSSPVTKSFNR |
| | | | | | GEC |

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1-59. (canceled)

60. An antibody conjugate represented by the structure of Formula (I): 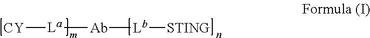
or a pharmaceutically acceptable salt, solvate, tautomer, stereoisomer, regioisomer, or mixture of regioisomers thereof;
wherein Ab is an anti-TROP2 antibody or antigen-binding fragment thereof;
subscript n is an integer selected from 1 to 10;
subscript m is an integer selected from 1 to 10;
-[L^{b}-STING]ₙ is a structure selected from: 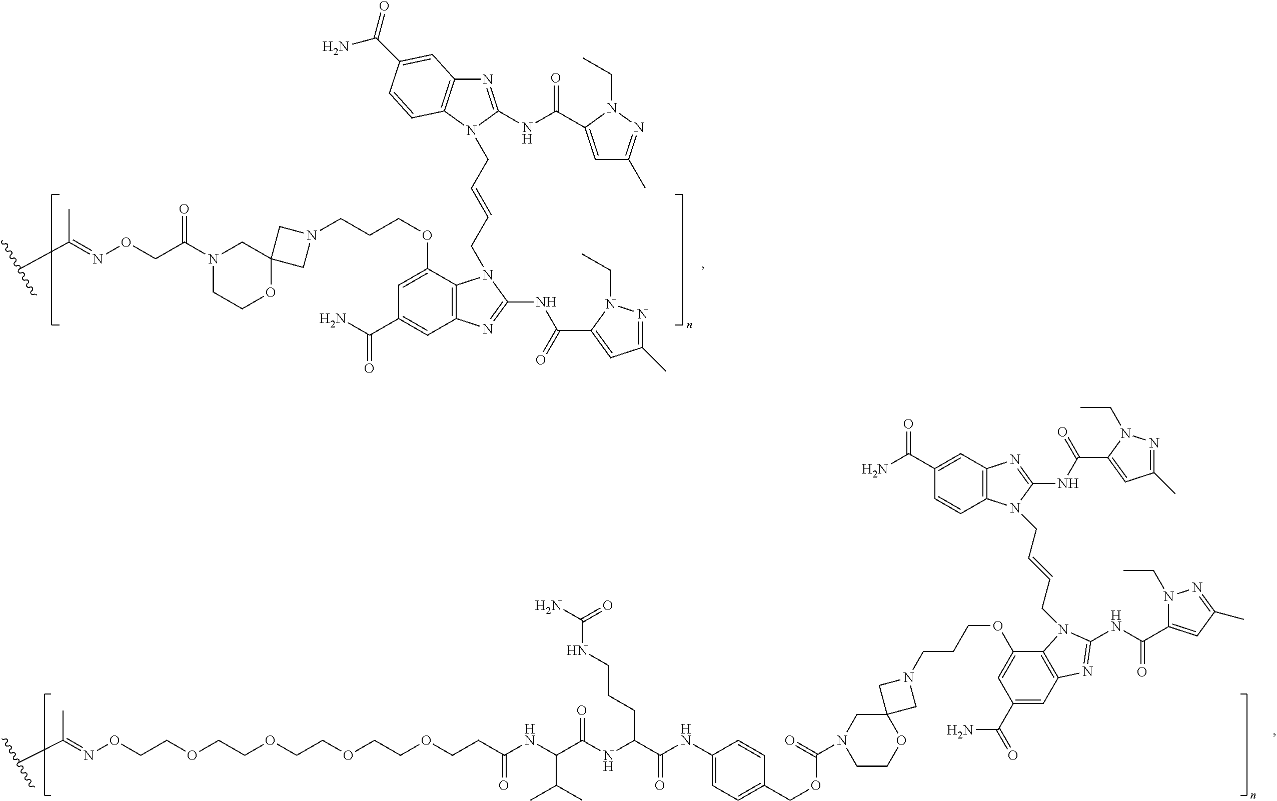 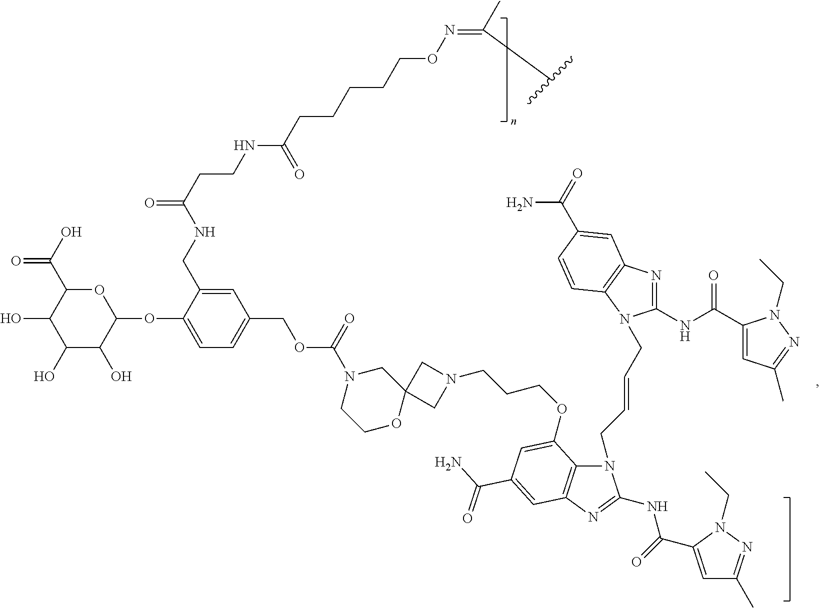 [SMILES: */C(C)=N\OCCCCCC(=O)NCCC(=O)NCc1cc(COC(=O)N2CCOC3(CN(CCCOc4cc(C(N)=O)cc5nc(NC(=O)c6cc(C)nn6CC)n(C/C=C/Cn6c(NC(=O)c7cc(C)nn7CC)nc7cc(C(N)=O)ccc76)c45)C3)C2)ccc1OC1OC(C(=O)O)C(O)C(O)C1O] 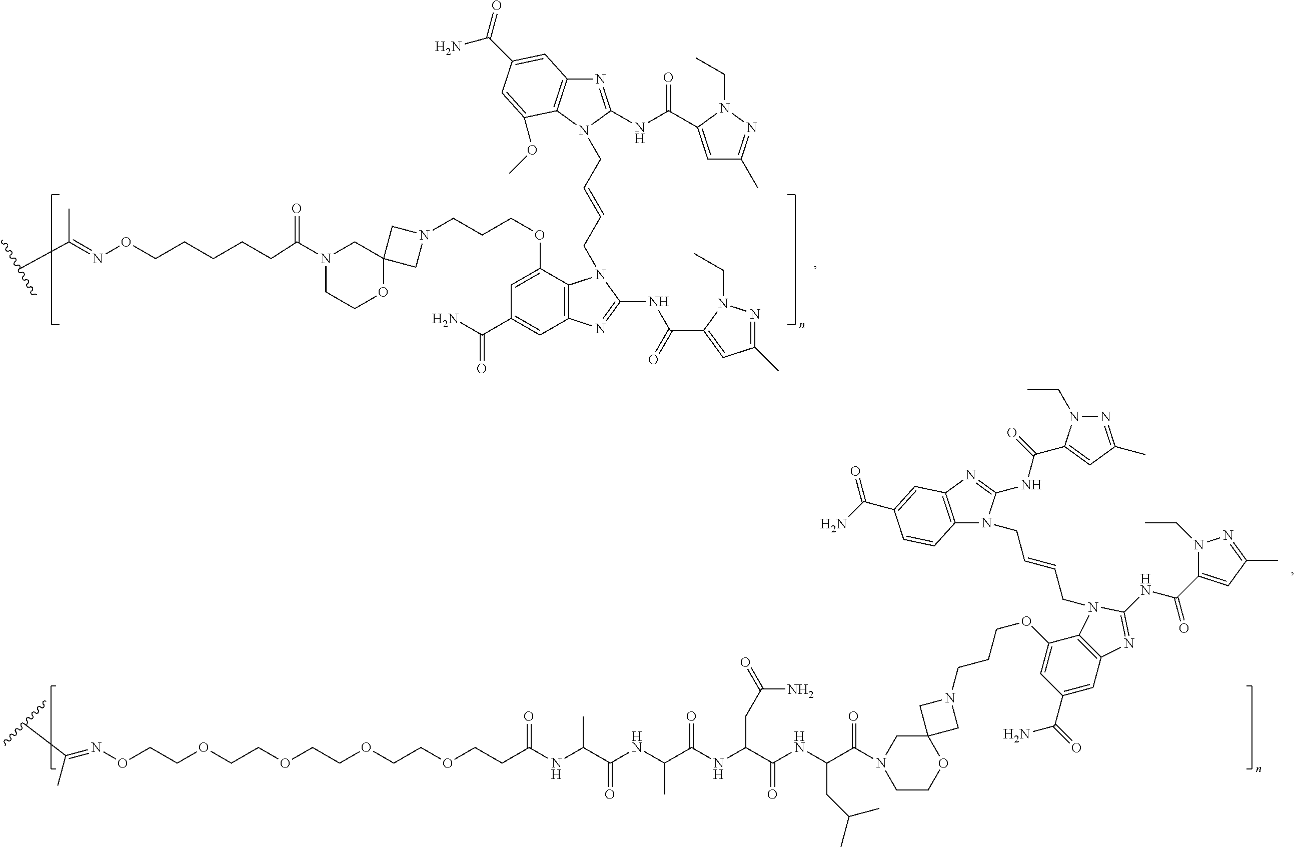 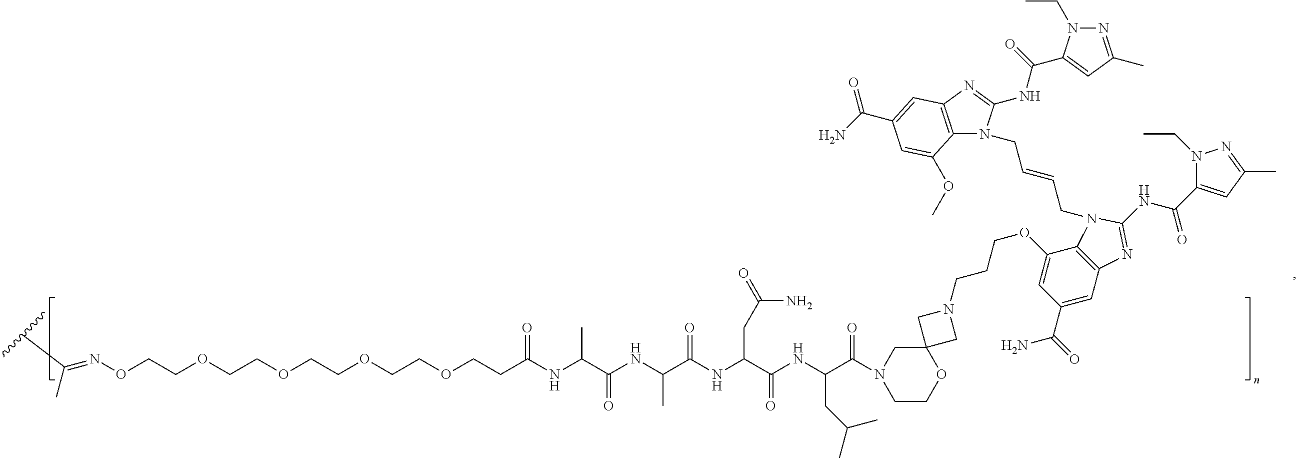 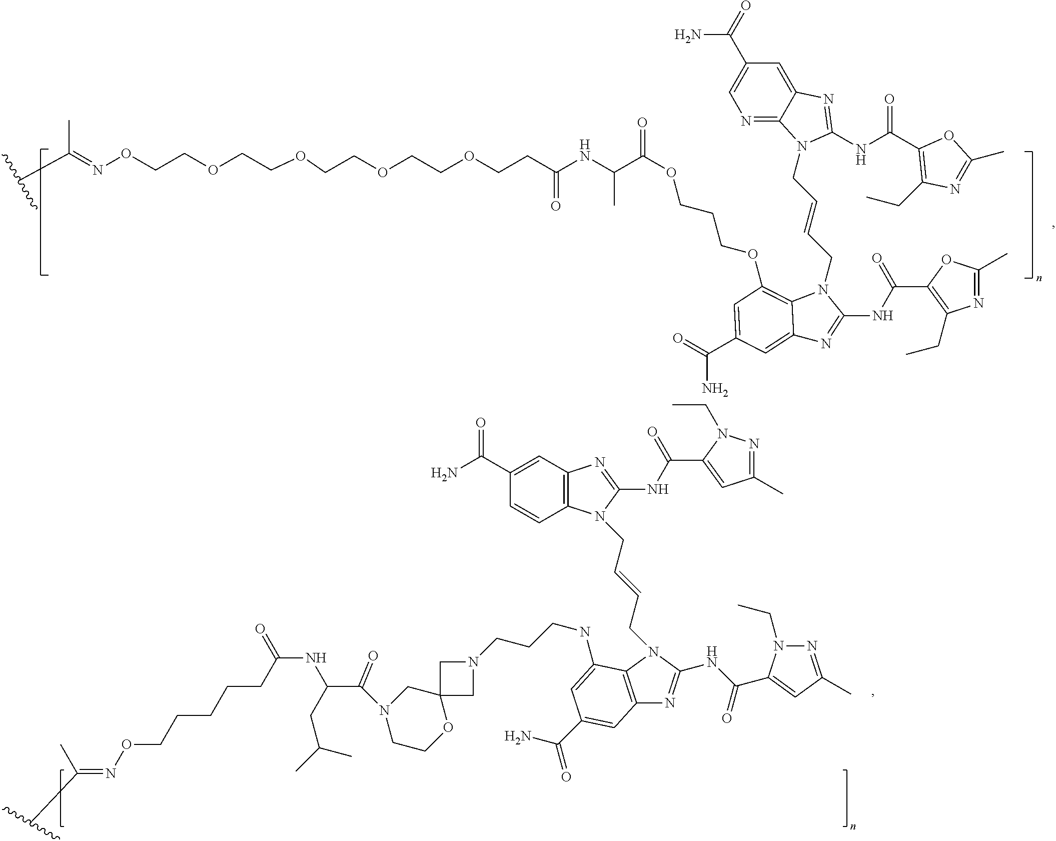 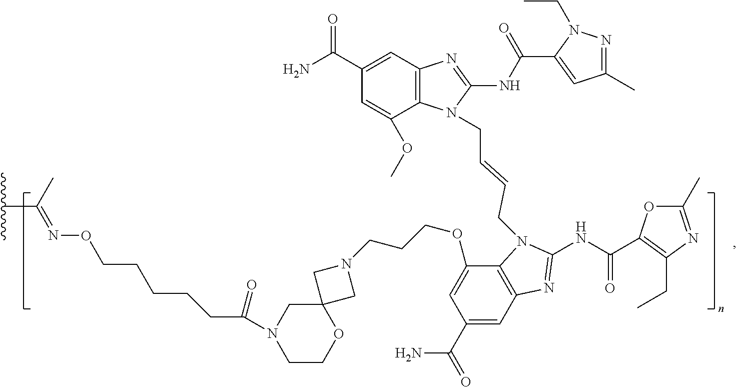 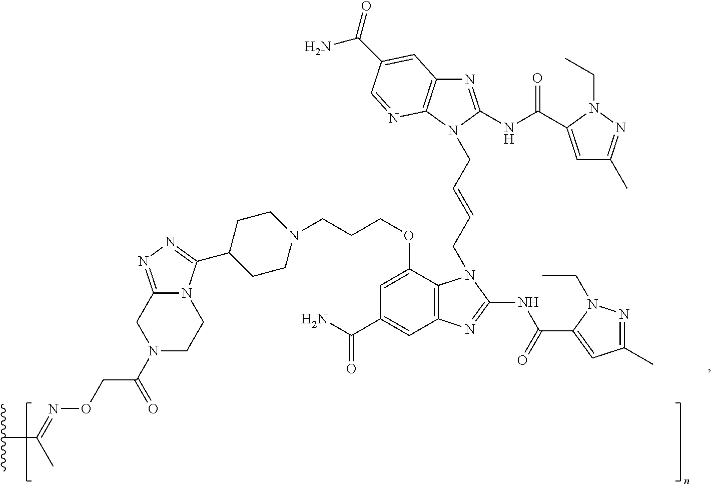 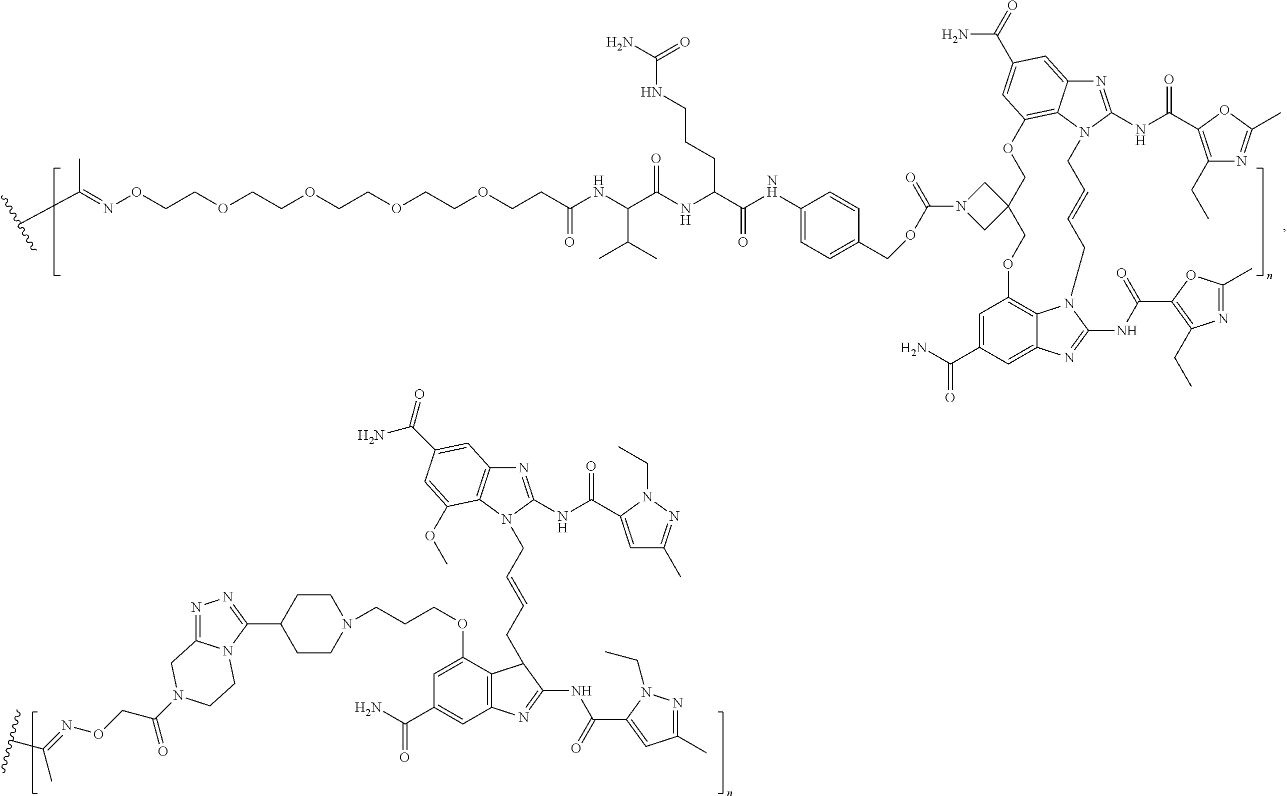 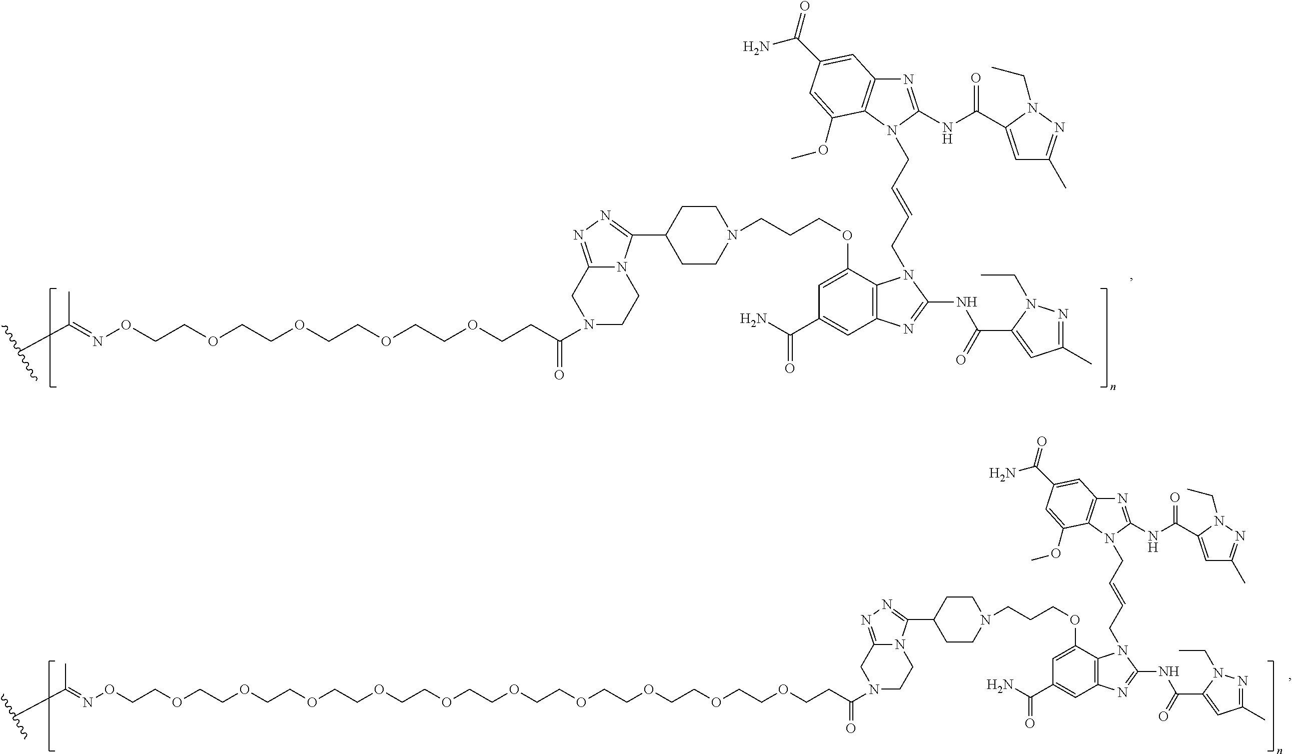 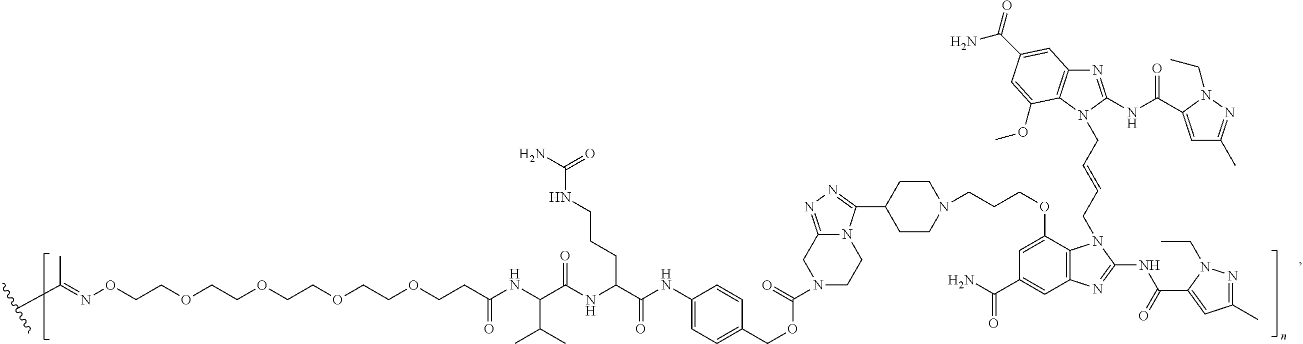 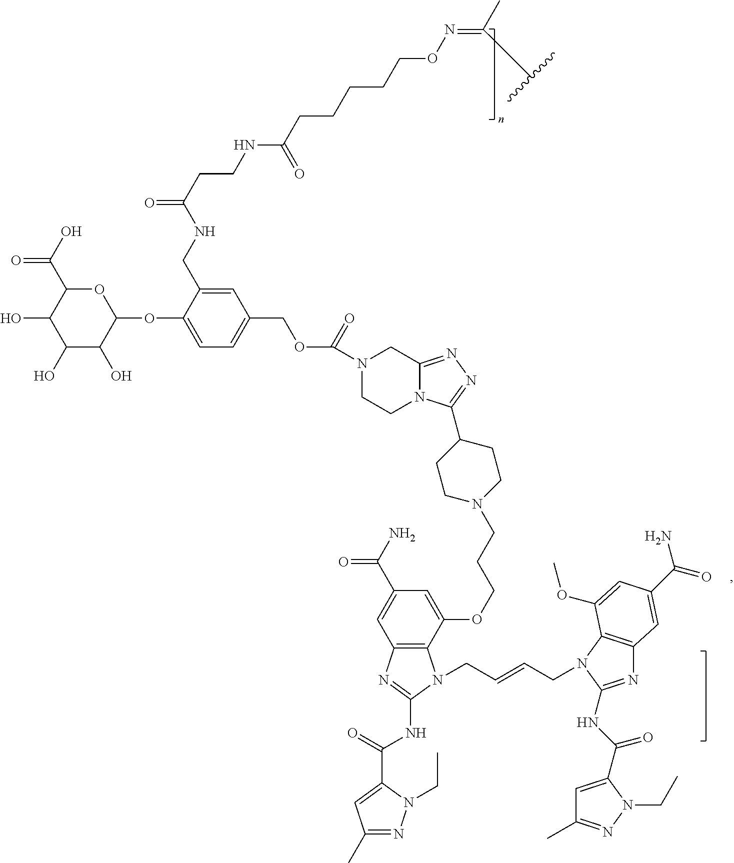 [SMILES: */C(C)=N\OCCCCCC(=O)NCCC(=O)NCc1cc(COC(=O)N2CCn3c(nnc3C3CCN(CCCOc4cc(C(N)=O)cc5nc(NC(=O)c6cc(C)nn6CC)n(C/C=C/Cn6c(NC(=O)c7cc(C)nn7CC)nc7cc(C(N)=O)cc(OC)c76)c45)CC3)C2)ccc1OC1OC(C(=O)O)C(O)C(O)C1O] 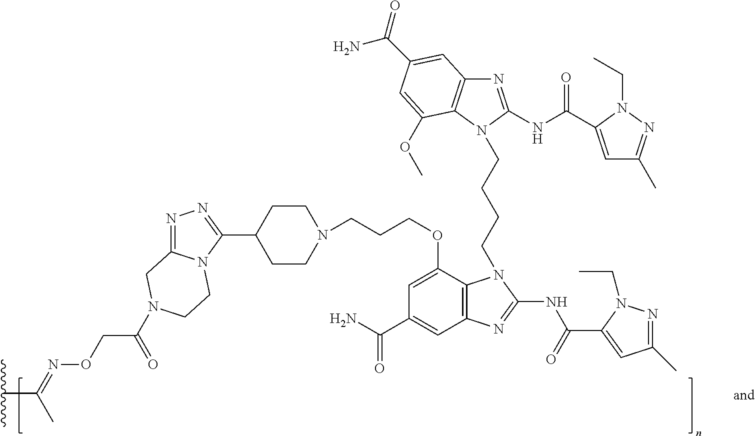 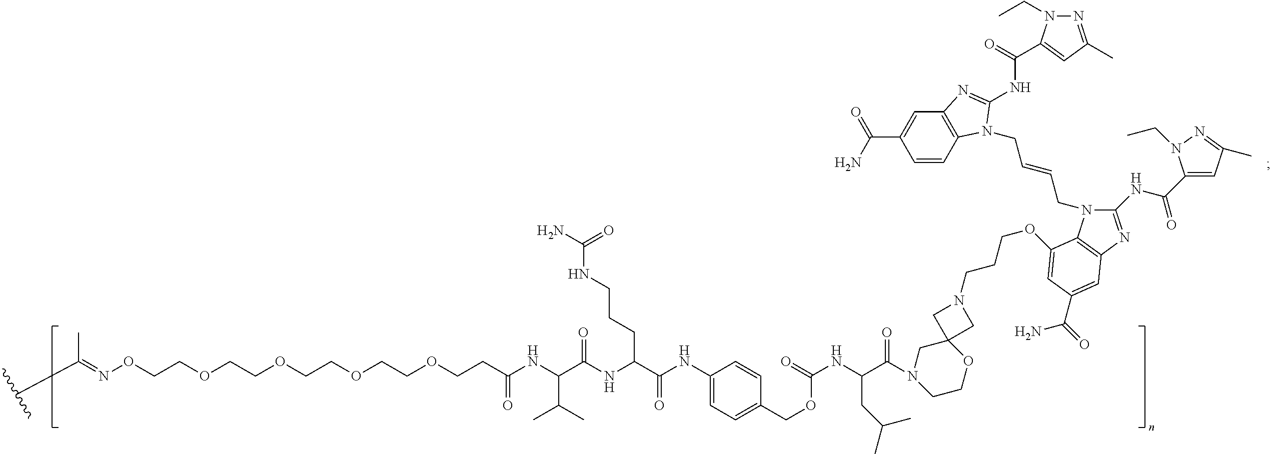
and
[CY-L^{a}-]ₘ- is a structure selected from: 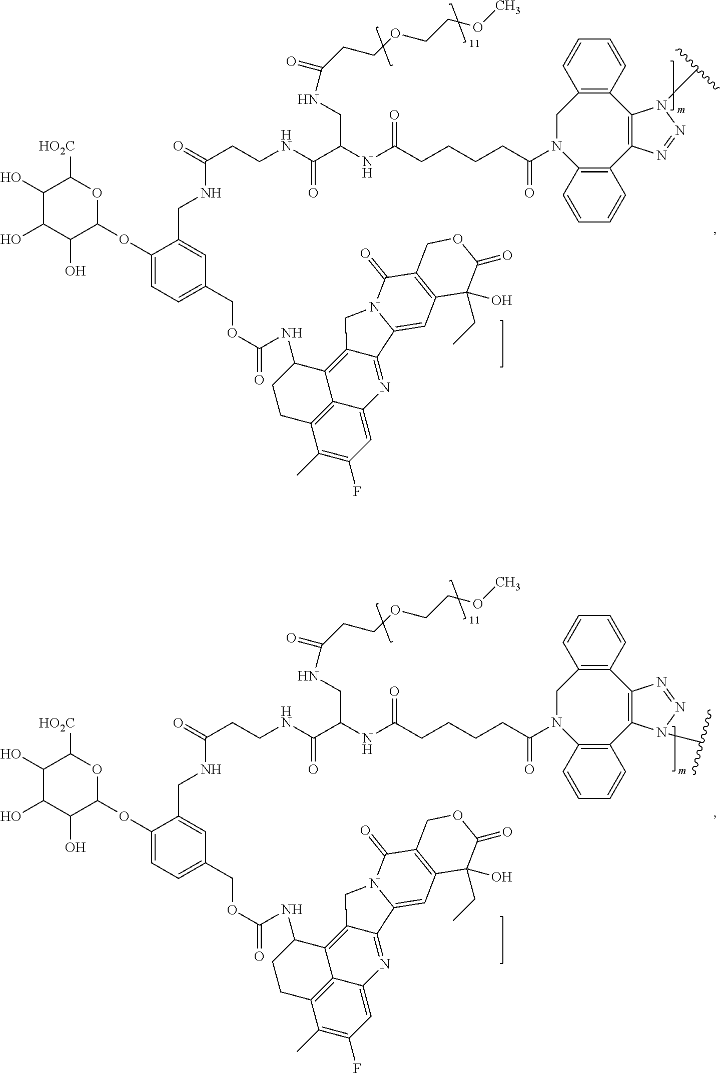 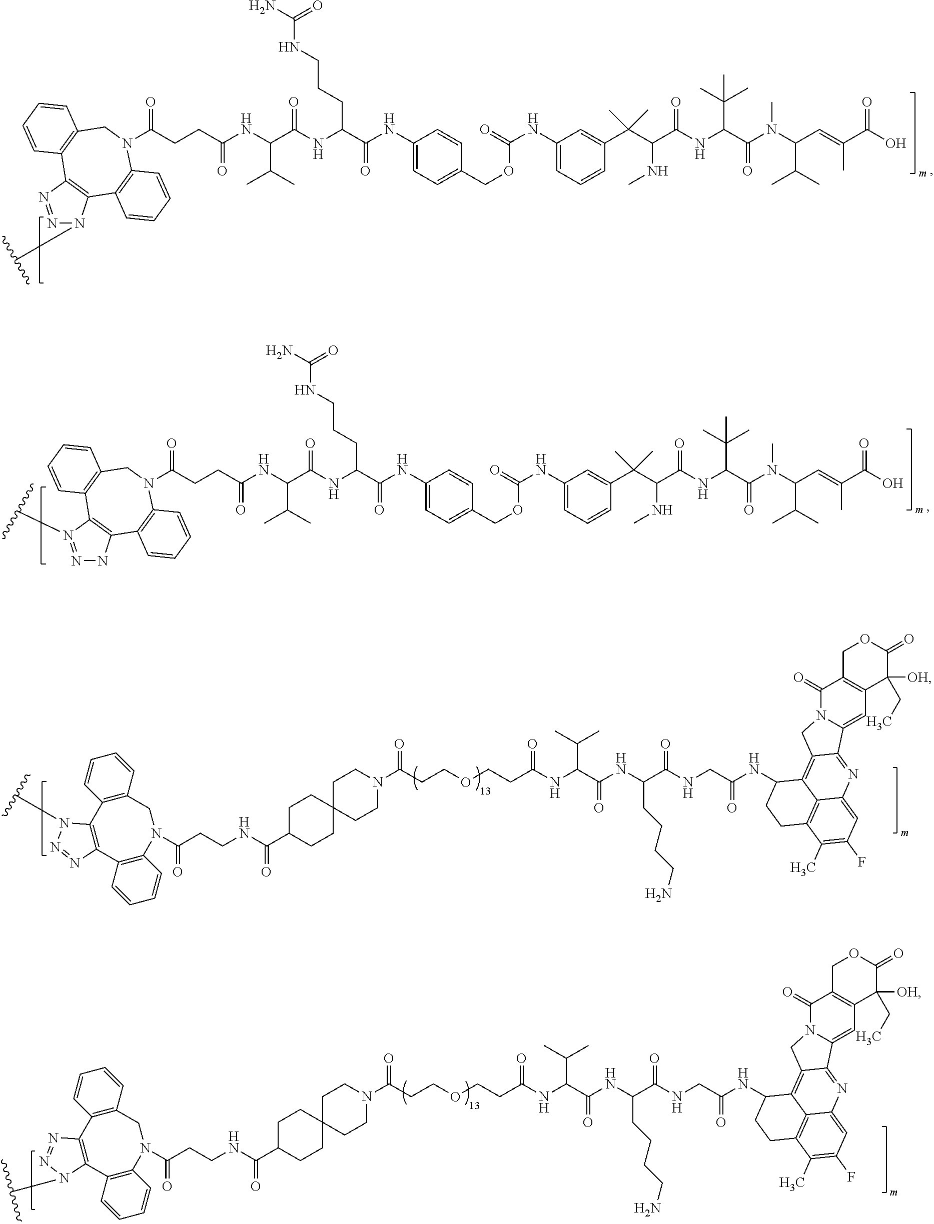 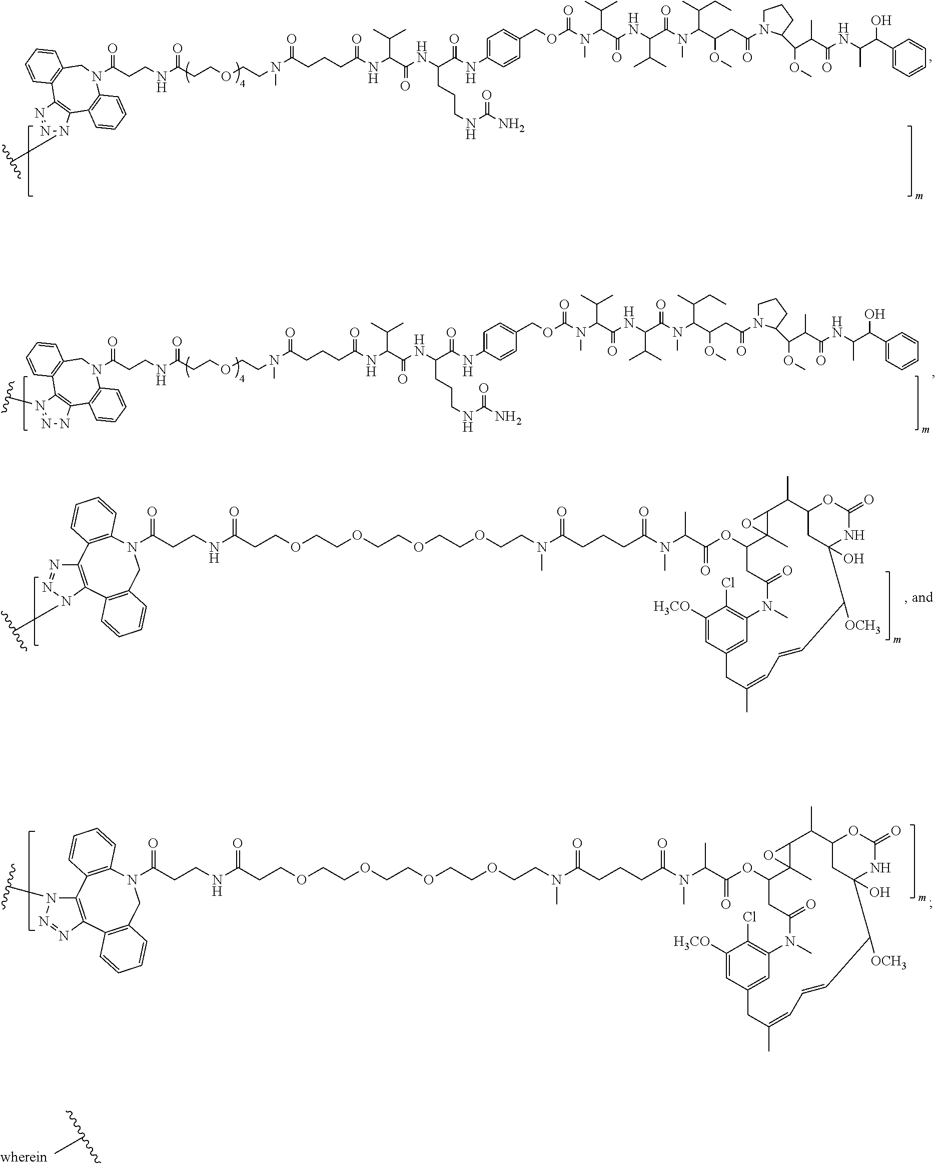
is the point of attachment to the rest of the formula.

61. The antibody conjugate of claim 60, wherein
[CY-L^{a}-]ₘ- is 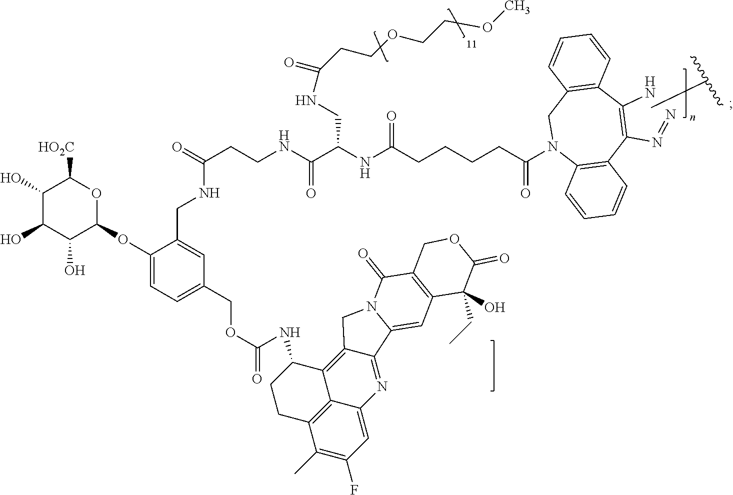
and -[L^{b}-STING]ₙ is

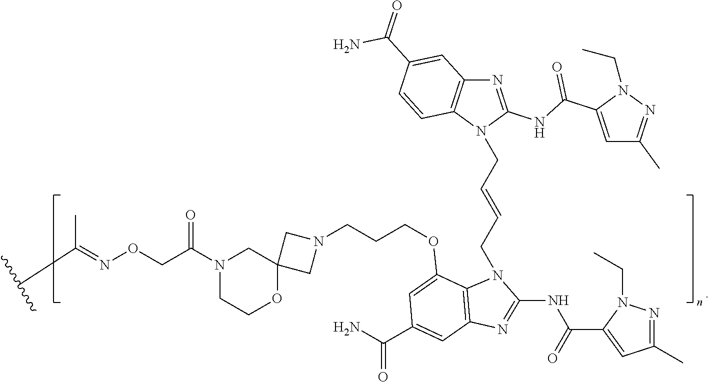

62. The antibody conjugate of claim 60, wherein
[CY-L^{a}-]ₘ- is 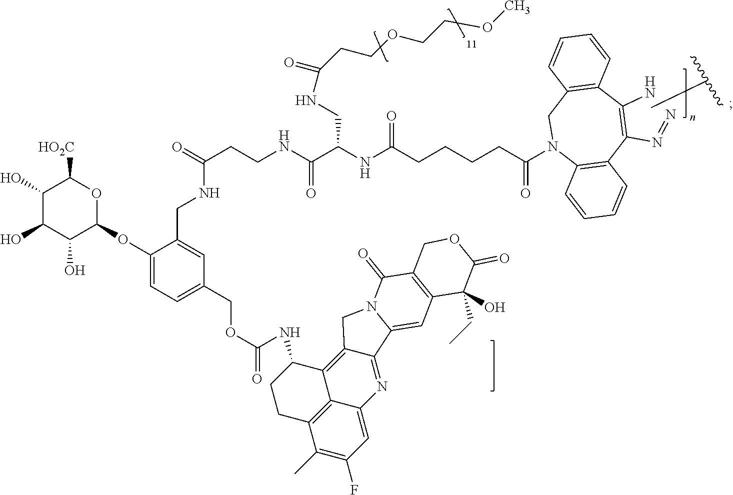
and -[L^{b}-STING]ₙ is a structure selected from

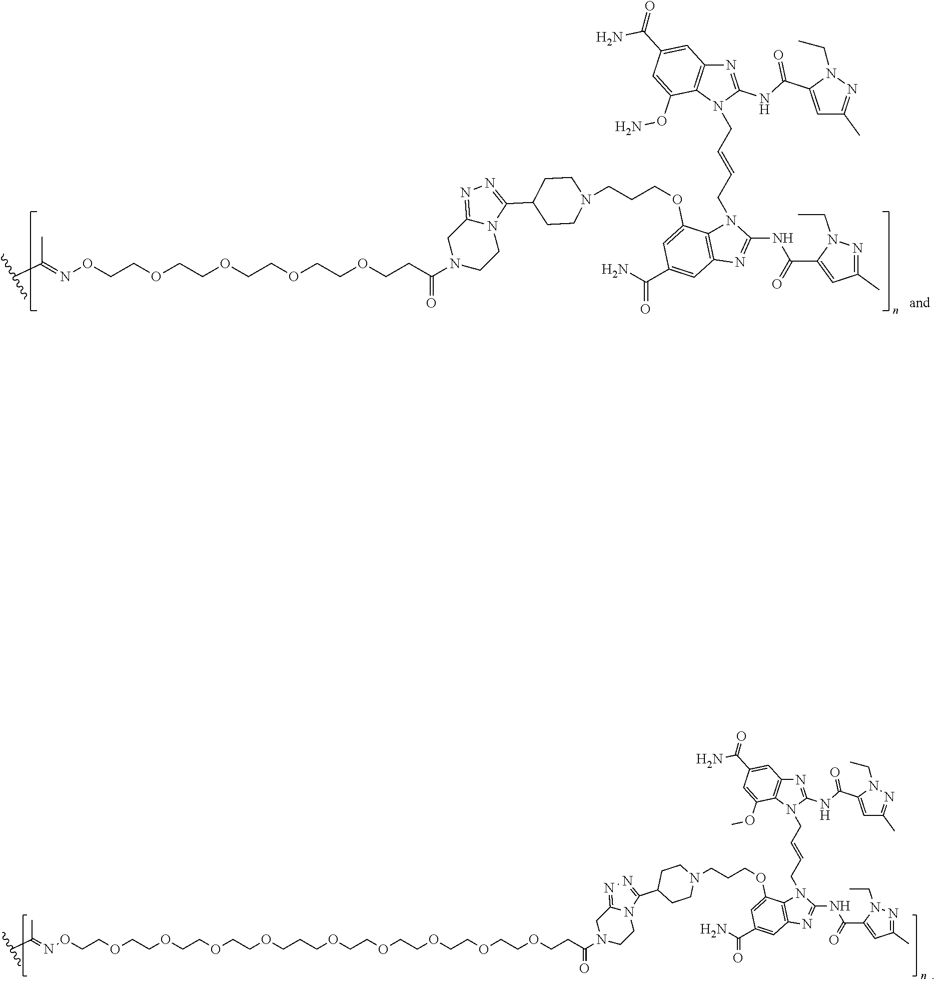

63. The antibody conjugate of claim 60, wherein m and n is an integer independently between 1 and 8, inclusive.

64. The antibody conjugate of claim 63, wherein the molar ratio of CY to STING is about 2.1.

65. The antibody conjugate of claim 64, wherein the anti-TROP2 antibody or the antigen-binding fragment thereof comprises at least two modified amino acid residues suitable for conjugation that are covalently linked to L^{a} and/or L^{b}.

66. The antibody conjugate of claim 60, wherein (a) the anti-TROP2 antibody or antigen-binding fragment thereof is covalently linked to L^{a} via a modified amino acid and (b) the anti-TROP2 antibody or antigen binding fragment thereof is covalently linked to L^{b} via a modified amino acid that is different than the modified amino acid in (a).

67. The antibody conjugate of claim 65, wherein the modified amino acid residue is selected from the group consisting of p-acetyl-L-phenylalanine, O-methyl-L-tyrosine, 3-methyl-phenylalanine, O-4-allyl-L-tyrosine, 4-propyl-L-tyrosine, fluorinated phenylalanine, isopropyl-L-phenylalanine, p-azido-L-phenylalanine, p-acyl-L-phenylalanine, p-A benzoyl-L-phenylalanine, p-iodo-phenylalanine, p-bromophenylalanine, p-amino-L-phenylalanine, isopropyl-L-phenylalanine, p-propargyloxy-phenylalanine, and p-azidomethyl-L-phenylalanine.

68. The antibody conjugate of claim 67, wherein the modified amino acid residue is independently selected from p-azidomethyl-L-phenylalanine and p-acetyl-L-phenylalanine.

69. The antibody conjugate of claim 68, wherein the modified amino acid residues are at positions selected from the group consisting of heavy chain or light chain residues HC-F404, HC-K121, HC-Y180, HC-F241, HC-221, LC-T22, LC-S7, LC-N152, LC-K42, LC-E161, LC-D170, HC-S136, HC-S25, HC-A40, HC-S119, HC-S190, HC-K222, HC-R19, HC-Y52, or HC-S70, according to the Kabat or Chothia or EU numbering scheme, or a post translationally modified variant thereof.

70. The antibody conjugate of claim 69, wherein the modified amino acid residues are at positions selected from the group consisting of heavy chain or light chain residues HC-F404, HC-F241, HC-Y180, and LC-K42.

71. The antibody conjugate of claim 70, wherein the modified amino acid residues are p-azidomethyl-L-phenylalanine at HC-F404, HC-F241, and/or HC-Y180, and p-acetyl-L-phenylalanine at LC-K42.

72. The antibody conjugate of claim 71, wherein [CY-L^{a}-]ₘ- is bonded to the p-azidomethyl-L-phenylalanine residues at HC-Y180 and HC-F404, and -[L_{b}-STING]ₙ is bonded to the p-acetyl-L-phenylalanine at LC-K42.

73. The antibody conjugate of claim 72, wherein the antibody comprises a heavy chain variable region and a light chain variable region,
wherein the heavy chain variable region comprises a CDR1 consisting of an amino acid sequence of SEQ ID NO: 4, a CDR2 consisting of an amino acid sequence of SEQ ID NO: 5, and a CDR3 consisting of an amino acid sequence of SEQ ID NO: 6, and the light chain variable region comprises a CDR1 consisting of an amino acid sequence of SEQ ID NO: 7, a CDR2 consisting of an amino acid sequence of SEQ ID NO: 8, and a CDR3 consisting of an amino acid sequence of SEQ ID NO: 9.

74. The antibody conjugate of claim 73, wherein the antibody comprises a heavy chain variable region and a light chain variable region,
wherein the heavy chain variable region comprises an amino acid sequence of SEQ ID NO: 10 and the light chain variable region comprises an amino acid sequence of SEQ ID NO: 11.

75. The antibody conjugate of claim 74, wherein the antibody comprises either one of the following heavy chain and light chain sequences:
(a) a heavy chain consisting of an amino acid sequence of SEQ ID NO: 12 and a light chain consisting of an amino acid sequence of SEQ ID NO: 14,
(b) a heavy chain consisting of an amino acid sequence of SEQ ID NO: 13 and a light chain consisting of an amino acid sequence of SEQ ID NO: 14,
(c) a heavy chain consisting of an amino acid sequence of SEQ ID NO: 15 and a light chain consisting of an amino acid sequence of SEQ ID NO: 17, or
(d) a heavy chain consisting of an amino acid sequence of SEQ ID NO: 16 and a light chain consisting of an amino acid sequence of SEQ ID NO: 17.

76. The antibody conjugate of claim 75, wherein the antibody comprises the following heavy chain and light chain sequences:
(a) a heavy chain consisting of an amino acid sequence of SEQ ID NO: 12 and a light chain consisting of an amino acid sequence of SEQ ID NO: 14.

77. A pharmaceutical composition comprising an antibody conjugate of claim 60 and a pharmaceutically acceptable carrier, excipient, or diluent.

78. A method of treating abnormal cellular proliferation in a subject in need thereof comprising administering an antibody conjugate of claim 60, wherein the abnormal cellular proliferation is cancer.

79. The method of claim 78, wherein the cancer is selected from lymphoma, head and neck squamous cell carcinoma, urothelial cancer, lung cancer, breast cancer, prostate cancer, SCLC, bladder cancer, and melanoma.
